(19) United States
(12) Patent Application Publication
(10) Pub. No.: US 2011/0230476 A1
(43) Pub. Date: Sep. 22, 2011

(54) PI3 KINASE INHIBITORS AND USES THEREOF

(75) Inventors: DEQIANG NIU, LEXINGTON, MA (US); RUSSELL C. PETTER, STOW, MA (US); JUSWINDER SINGH, ASHLAND, MA (US); ARTHUR F. KLUGE, LINCOLN, MA (US); HORMOZ MAZDIYASNI, MARLBOROUGH, MA (US); ZHENDONG ZHU, WESTBOROUGH, MA (US); LIXIN QIAO, ANDOVER, MA (US); KEVIN KUNTZ, WOBURN, MA (US)

(73) Assignee: AVILA THERAPEUTICS, INC., WALTHAM, MA (US)

(21) Appl. No.: 12/878,965

(22) Filed: Sep. 9, 2010

Related U.S. Application Data
(60) Provisional application No. 61/371,396, filed on Aug. 6, 2010.
(60) Provisional application No. 61/240,947, filed on Sep. 9, 2009.

(51) Int. Cl. A61K 31/538; C07D 413/14; A61K 31/5377; C07D 487/04; A61K 31/519; C07D 401/14; A61K 31/4709; C07D 417/14; A61K 31/4725; A61K 31/496; C07D 495/04; A61P 35/00; C12N 9/99; C12N 9/96

(52) U.S. Cl. 514/230.5; 544/121; 514/235.8; 544/70; 544/262; 514/262.1; 546/167; 514/314; 544/118; 514/234.2; 544/281; 514/259.1; 546/146; 514/307; 544/363; 514/253.06; 544/278; 514/260.1; 544/105; 435/184; 435/188

(57) ABSTRACT

The present invention provides compounds, compositions thereof, and methods of using the same.

### CROSS REFERENCE TO RELATED APPLICATIONS

The present application claims priority to U.S. provisional application Ser. No. 61/240,947, filed Sep. 9, 2009, and U.S. provisional application Ser. No. 61/371,396, filed Aug. 6, 2010, the entirety of each of which is hereby incorporated by reference.

### TECHNICAL FIELD OF THE INVENTION

The present invention relates to compounds useful as inhibitors of PI3 kinase. The invention also provides pharmaceutically acceptable compositions comprising compounds of the present invention and methods of using said compositions in the treatment of various disorders.

### SEQUENCE LISTING

In accordance with 37 CFR 1.52(e)(5), a Sequence Listing in the form of a text file (entitled “Sequence_listing.txt,” created on Nov. 16, 2010, and 4 kilobytes) is incorporated herein by reference in its entirety.

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is the phosphatidylinositol 3-kinase superfamily.

Phosphatidylinositol 3-kinases (PI3Ks) belong to the large family of PI3K-related kinases. PI3Ks phosphorylate lipid molecules, rather than proteins, and are consequently known as lipid kinases. Specifically, PI3Ks phosphorylate the 3′-OH position of the inositol ring of phosphatidyl inositides. Class I PI3Ks are of particular interest and are further divided into Class IA and Class IB kinases based on sequence homology and substrate specificity. Class IA PI3Ks contain a p85 regulatory subunit that heterodimerizes with a p110α, p110β, or p110δ catalytic subunit. These kinases are commonly known as PI3Kα, PI3Kβ, and PI3Kδ and are activated by receptor tyrosine kinases. The Class IB PI3K contains a p110γ catalytic subunit and is commonly known as PI3Kγ. PI3Kγ is activated by heterotrimeric G-proteins. PI3Kα and PI3Kβ have a broad tissue distribution, while PI3Kδ and PI3Kγ are primarily expressed in leukocytes.

Class II and Class III PI3Ks are less well-known and well-studied than Class I PI3Ks. Class II comprises three catalytic isoforms: C2α, C2β, and C2γ. C2α and C2β are expressed throughout the body, while C2γ is limited to hepatocytes. No regulatory subunit has been identified for the Class II PI3Ks. Class III PI3Ks exist as heterodimers of p150 regulatory subunits and Vps34 catalytic subunits, and are thought to be involved in protein trafficking.

Closely related to the PI3Ks are phophatidylinositol 4-kinases (PI4Ks), which phosphorylate the 4′-OH position of phosphatidylinositides. Of the four known PI4K isoforms, PI4KA, also known as PI4KIIIα, is the mostly closely related to PI3Ks.

In addition to the classical PI3 kinases, there is a group of “PI3K-related kinases,” sometimes known as Class IV PI3Ks. Class IV PI3Ks contain a catalytic core similar to the PI3Ks and PI4Ks. These members of the PI3K superfamily are serine/threonine protein kinases and include ataxia telangiectasia mutated (ATM) kinase, ataxia telangiectasia and Rad3 related (ATR) kinase, DNA-dependent protein kinase (DNA-PK) and mammalian Target of Rapamycin (mTOR).

Many diseases are associated with abnormal cellular responses triggered by such kinase-mediated events as those described above. Such diseases include, but are not limited to, autoimmune diseases, inflammatory diseases, proliferative diseases, bone diseases, metabolic diseases, neurological and neurodegenerative diseases, cancer, cardiovascular diseases, allergies and asthma, Alzheimer's disease, and hormone-related diseases. Accordingly, there remains a need to find inhibitors of PI3Ks and related enzymes useful as therapeutic agents.

### BRIEF DESCRIPTION OF THE DRAWINGS

FIG. 1 depicts the results of provided compounds in a “washout” experiment in HCT116 cells as compared with known reversible inhibitors GSK-615 and GDC-941.

FIG. 2 depicts the results of compound II-a-16 in a “washout” experiment in PC3 cells as compared with known reversible inhibitor GDC-941.

FIG. 3 depicts the results of compounds II-a-144 and II-a-148 in a “washout” experiment as compared with three reversible reference compounds.

FIG. 4 depicts MS analysis confirming covalent modification of PI3Kα by compound II-a-45.

FIG. 5 depicts MS analysis confirming covalent modification of PI3Kα by compound II-a-49.

FIG. 6 depicts MS analysis confirming covalent modification of PI3Kα by compound II-a-3.

FIG. 7 depicts MS analysis confirming covalent modification of PI3Kα by compound II-a-144.

FIG. 8 depicts MS analysis confirming covalent modification of PI3Kα by compound II-a-148.

FIG. 9 depicts MS analysis after trypsin digestion confirming covalent modification of peptide ⁸⁵³NSHTIMQIQCK⁸⁶³ (SEQ ID NO:14) on PI3Kα by compound II-a-3.

FIG. 10 depicts MS/MS analysis confirming covalent modification of Cys-862 on PI3Kα by compound II-a-3.

FIG. 11 depicts MS analysis after trypsin digestion confirming covalent modification of peptide ⁸⁵³NSHTIMQIQCK⁸⁶³ (SEQ ID NO:14) on PI3Kα by compound II-a-144.

FIG. 12 depicts MS/MS analysis confirming covalent modification of Cys-862 on PI3Kα by compound II-a-144.

FIG. 13 depicts p-AKT^{Ser473} levels in mouse spleens treated with II-a-3 as compared to known reversible inhibitor GDC-941.

FIG. 14 depicts results from a SKOV3 tumor growth inhibition experiment with II-a-3 and II-a-148 compared with known reversible inhibitor GDC-941 as well as paclitaxel.

FIG. 15 depicts dose response target occupancy data for II-a-148 in SKOV3 cells as compared to known reversible inhibitor GDC-941.

FIG. 16 depicts MS analysis confirming covalent modification of PI3Kα by compound XII-54.

### DETAILED DESCRIPTION OF CERTAIN EMBODIMENTS

### 1. General Description of Compounds of the Invention

In certain embodiments, the present invention provides irreversible inhibitors of one or more PI3 Kinases and conjugates thereof. In some embodiments, such compounds include those of formulae I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, and XII-e:

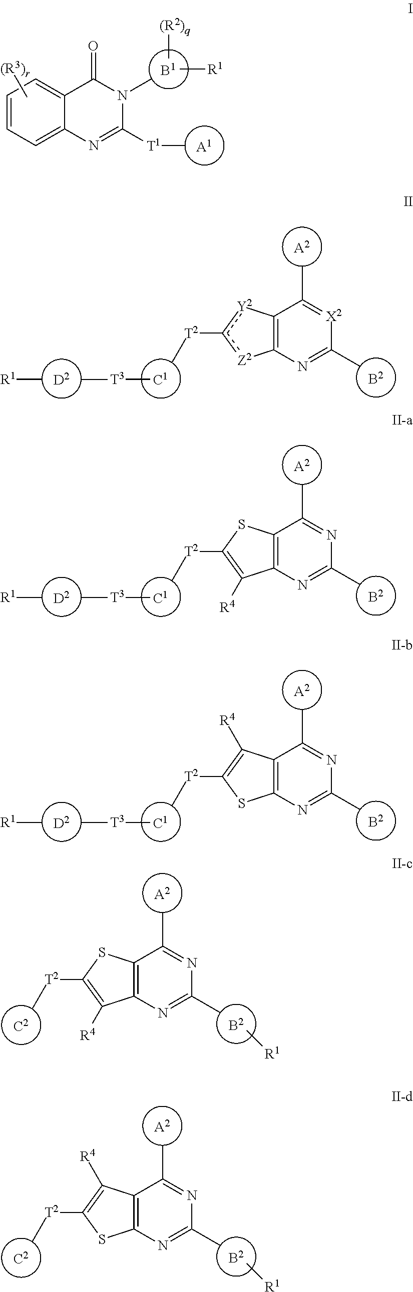

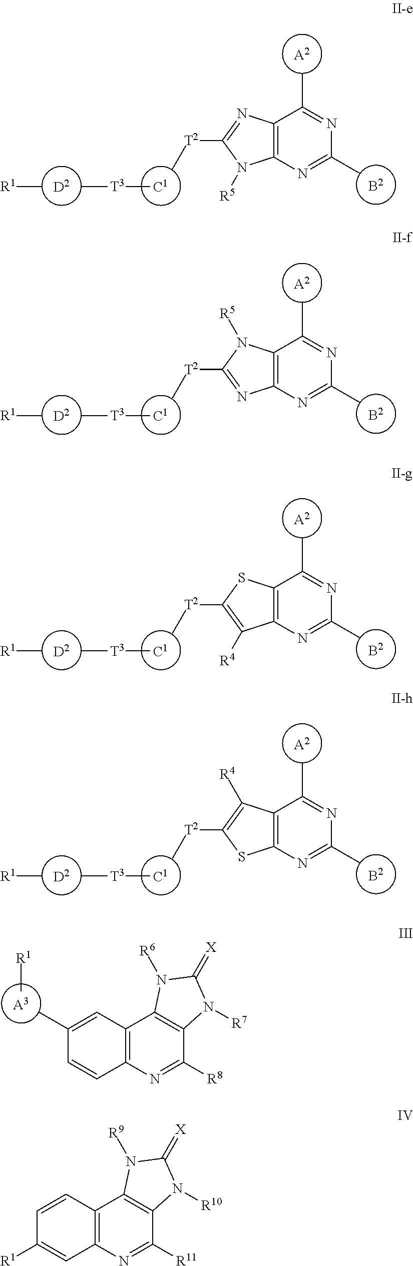

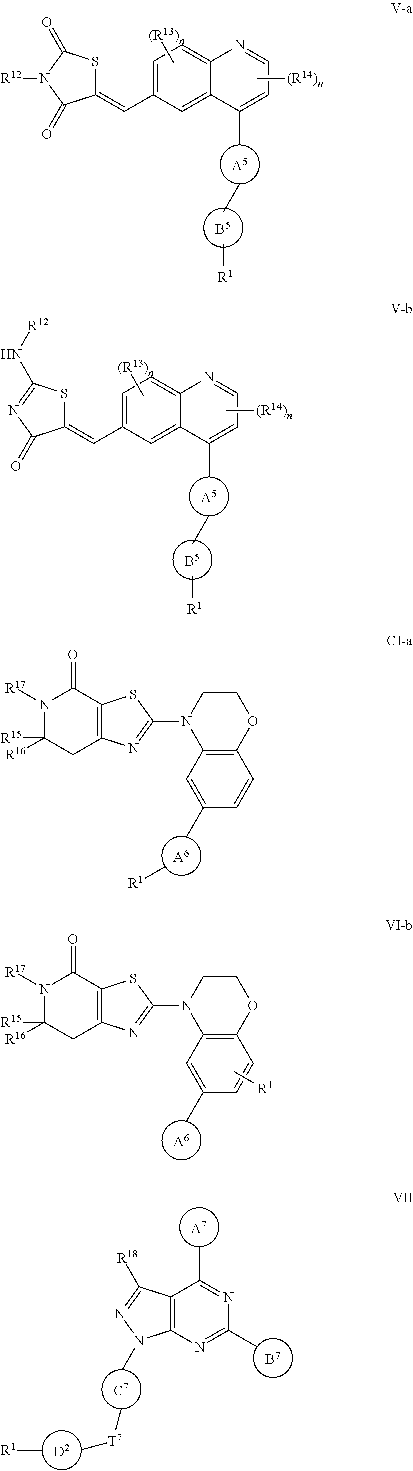

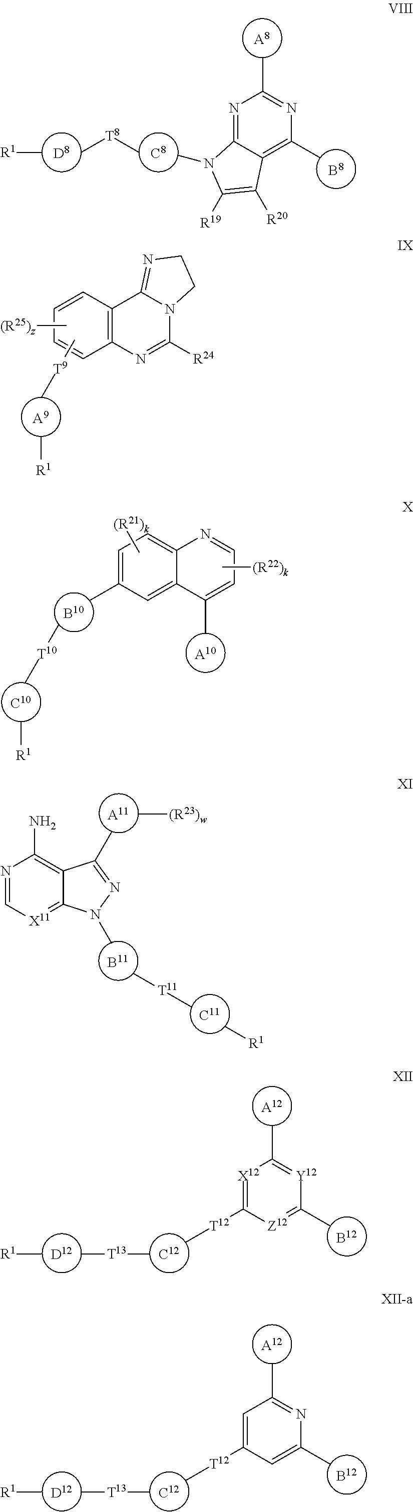

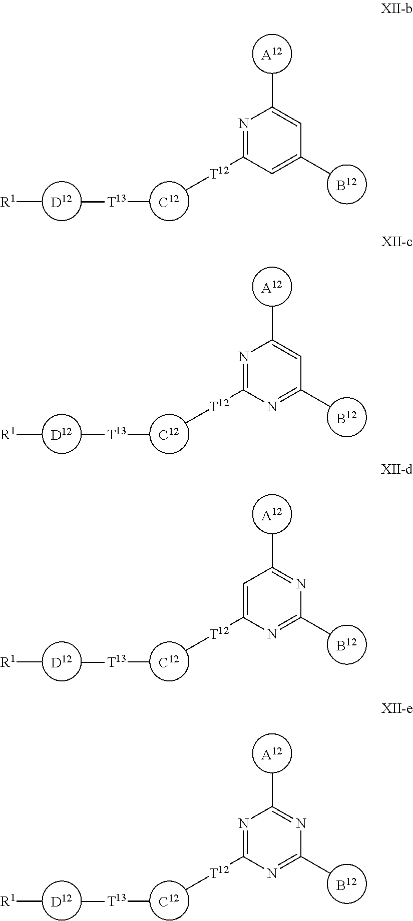

or a pharmaceutically acceptable salt thereof, wherein each variable is as defined and described herein.

### 2. Compounds and Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “carbocyclic”, “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “carbocyclic” (or “cycloaliphatic” or “carbocycle” or “cycloalkyl”) refers to a monocyclic C₃-C₈ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

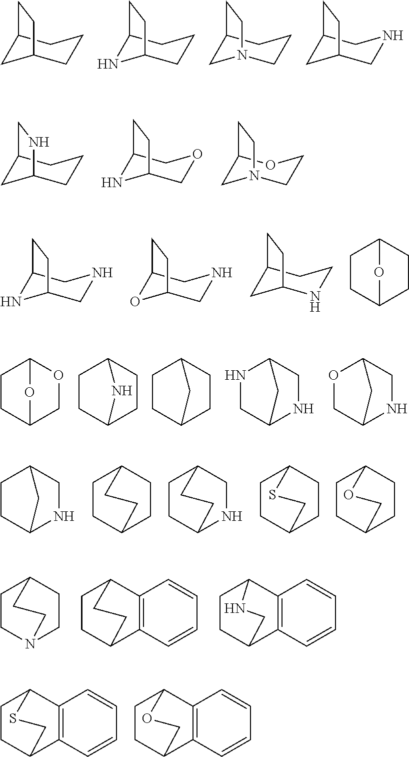

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

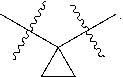

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In certain embodiments, a warhead moiety, R¹, of a provided compound comprises one or more deuterium atoms.

As used herein, the term “irreversible” or “irreversible inhibitor” refers to an inhibitor (i.e. a compound) that is able to be covalently bonded to a PI3 kinase in a substantially non-reversible manner. That is, whereas a reversible inhibitor is able to bind to (but is generally unable to form a covalent bond with) a PI3 kinase, and therefore can become dissociated from the a PI3 kinase an irreversible inhibitor will remain substantially bound to a PI3 kinase once covalent bond formation has occurred. Irreversible inhibitors usually display time dependency, whereby the degree of inhibition increases with the time with which the inhibitor is in contact with the enzyme. In certain embodiments, an irreversible inhibitor will remain substantially bound to a PI3 kinase once covalent bond formation has occurred and will remain bound for a time period that is longer than the life of the protein.

Methods for identifying if a compound is acting as an irreversible inhibitor are known to one of ordinary skill in the art. Such methods include, but are not limited to, enzyme kinetic analysis of the inhibition profile of the compound with PI3 kinase, the use of mass spectrometry of the protein drug target modified in the presence of the inhibitor compound, discontinuous exposure, also known as “washout,” experiments, and the use of labeling, such as radiolabelled inhibitor, to show covalent modification of the enzyme, as well as other methods known to one of skill in the art.

One of ordinary skill in the art will recognize that certain reactive functional groups can act as “warheads.” As used herein, the term “warhead” or “warhead group” refers to a functional group present on a compound of the present invention wherein that functional group is capable of covalently binding to an amino acid residue (such as cysteine, lysine, histidine, or other residues capable of being covalently modified) present in the binding pocket of the target protein, thereby irreversibly inhibiting the protein. It will be appreciated that the -L-Y group, as defined and described herein, provides such warhead groups for covalently, and irreversibly, inhibiting the protein.

As used herein, the term “inhibitor” is defined as a compound that binds to and/or inhibits PI3 kinase with measurable affinity. In certain embodiments, an inhibitor has an IC₅₀ and/or binding constant of less about 50 μM, less than about 1 μM, less than about 500 nM, less than about 100 nM, less than about 10 nM, or less than about 1 nM.

The terms “measurable affinity” and “measurably inhibit,” as used herein, means a measurable change in a PI3 kinase activity between a sample comprising a compound of the present invention, or composition thereof, and a PI3 kinase, and an equivalent sample comprising a PI3 kinase, in the absence of said compound, or composition thereof.

### 3. Description of Exemplary Embodiments

As described herein, the present invention provides irreversible inhibitors of one or more PI3 kinases. Such compounds comprising a warhead group, designated as R¹, include those of formulae I, II, II-a, II-b, II-c, II-d, II-e, II-f, II-g, II-h, III, IV, V-a, V-b, VI-a, VI-b, VII, VIII, IX, X, XI, XII, XII-a, MI-b, XII-c, XII-d, and XII-e as described herein. Without wishing to be bound by any particular theory, it is believed that such R¹ groups, i.e. warhead groups, are particularly suitable for covalently binding to a key cysteine residue in the binding domain of a PI3 kinase. One of ordinary skill in the art will appreciate that PI3 kinases, and mutants thereof (including, but not limited to Glu542, Glu545 and His1047 (Samuels et al., Science (2004) 304: 552)), have a cysteine residue in the binding domain. Without wishing to be bound by any particular theory, it is believed that proximity of a warhead group to the cysteine of interest facilitates covalent modification of that cysteine by the warhead group.

Cysteine residues of PI3 kinase family members targeted for covalent modification by irreversible inhibitors of the present invention include those summarized in Table 1, below, where the “Target” refers to the protein of interest; the “Sequence Code” refers to the residue numbering protocol in accordance with the ExPASy proteomics server of the Swiss Institute of Bioinformatics (www.expasy.org); the “Sequence” refers to an identifying portion of the Target's amino acid sequence which includes the cysteine of interest; and the “Residue #” refers to the cysteine residue number as set forth in the sequence code.

TABLE 1

| | Sequence | | Residue |
|---|---|---|---|
| Target | Code | Sequence | # |
| PI3K ALPHA | P42336 | QCKGGLKGAL | 862 |
| | | QFNSHTLHQW | |
| | | (SEQ ID NO: 1) | |
| MTOR | P42345 | PHCDTLHALI | 2243 |
| | | RDYREKKKIL | |
| | | (SEQ ID NO: 2) | |
| PI3K ALPHA | P42336 | LPYGCLS | 838 |
| | | (SEQ ID NO: 3) | |
| PI3K GAMMA | P48736 | LPYGCIS | 869 |
| | | (SEQ ID NO: 4) | |
| PI3K DELTA | O00329 | TPYGCLP | 815 |
| | | (SEQ ID NO: 5) | |
| PI3K BETA, | P42338 | LPYGCLA | 841 |
| CLASS 1A | | (SEQ ID NO: 6) | |
| PI3K BETA, | A2RUF7 | VIFRCFS | 1119 |
| CLASS 2 | | (SEQ ID NO: 7) | |
| DNA-PK | P78527 | NKDSKPPGNL | 3683 |
| | | KECSPWMSDF | |
| | | (SEQ ID NO: 8) | |
| ATM KINASE | Q13315 | SQRSGVLEWC | 2770 |
| | | TGTVPIGEFL | |
| | | (SEQ ID NO: 9) | |
| ATM KINASE | Q13315 | RNTETRKRKL | 2753 |
| | | TICTYKVVPL | |
| | | (SEQ ID NO: 10) | |
| PI4KA HUMAN | P42356 | TAPGCGVIEC | 1840 |
| | | IPDCTSRDQL | |
| | | (SEQ ID NO: 11) | |
| PI4KA HUMAN | P42356 | TAPGCGVIEC | 1844 |
| | | IPDCTSRDQL | |
| | | (SEQ ID NO: 12) | |
| PI4KA HUMAN | P42356 | GQKISWQAAI | 1797 |
| | | FKVGDDCRQD | |
| | | (SEQ ID NO: 13) | |

As is apparent from Table 1, above, cysteine residues of interest can also be described by an identifying portion of the Target's amino acid sequence which includes the cysteine of interest. Thus, in certain embodiments, one or more of the following characteristics apply:
Cys862 of PI3K-alpha is characterized in that Cys862 is the cysteine embedded in the amino acid sequence QCKGGLKGAL QFNSHTLHQW of PI3K-alpha;
Cys2243 of MTOR is characterized in that Cys2243 is the cysteine embedded in the amino acid sequence PHCDTLHALI RDYREKKKIL of MTOR;
Cys838 of PI3K-alpha is characterized in that Cys838 is the cysteine embedded in the amino acid sequence LPYGCLS of PI3K-alpha;
Cys869 of PI3K-gamma is characterized in that Cys869 is the cysteine embedded in the amino acid sequence LPYGCI S of PI3K-gamma;
Cys815 of PI3K-delta is characterized in that Cys815 is the cysteine embedded in the amino acid sequence TPYGCLP of PI3K-delta;
Cys841 of PI3K-beta, Class 1A, is characterized in that Cys841 is the cysteine embedded in the amino acid sequence LPYGCLA of PI3K-beta, Class 1A;
Cys1119 of PI3K-beta, Class 2, is characterized in that Cys1119 is the cysteine embedded in the amino acid sequence VIFRCFS of PI3K-beta, Class 2;
Cys3683 of DNA-PK is characterized in that Cys3683 is the cysteine embedded in the amino acid sequence NKDSKPPGNL KECSPWMSDF of DNA-PK;
Cys2770 of ATM-Kinase is characterized in that Cys2770 is the cysteine embedded in the amino acid sequence SQRSGVLEWCTGTVPIGEFL of ATM-kinase;
Cys2753 of ATM-Kinase is characterized in that Cys2770 is the cysteine embedded in the amino acid sequence RNTETRKRKLTICTYKVVPL of ATM-kinase;
Cys1840 of PI4KA is characterized in that Cys1840 is the cysteine embedded in the amino acid sequence TAPGCGVIECIPDCTSRDQL of PI4KA;
Cys1844 of PI4KA is characterized in that Cys1844 is the cysteine embedded in the amino acid sequence TAPGCGVIECIPDCTSRDQL of PI4KA; and/or
Cys1797 of PI4KA is characterized in that Cys1797 is the cysteine embedded in the amino acid sequence GQKISWQAAIFKVGDDCRQD of PI4KA.

Additionally, it will be appreciated that certain cysteine residues are conserved across PI3 kinase family members. Such cysteine residues are designated by Cys Group, as set forth in Table 1-a, below. Thus, for the purposes of clarity, the grouping of conserved cysteine residues is exemplified by Table 1-a, below.

TABLE 1-a

| Subtype | Cys1 | Cys2 | Cys3 | Cys4 | Cys5 | Cys6 | Cys7 | Cys8 | Cys9 |
|---|---|---|---|---|---|---|---|---|---|
| PI3Kα | ✓ | | ✓ | | | | | | |
| PI3Kβ- | | | ✓ | | | | | | |
| 1A | | | | | | | | | |
| PI3Kβ- | | | ✓ | | | | | | |
| 2 | | | | | | | | | |
| PI3Kγ | | | ✓ | | | | | | |
| PI3Kδ | | | ✓ | | | | | | |
| mTOR | | ✓ | | | | | | | |
| DNA- | | | | ✓ | | | | | |
| PK | | | | | | | | | |
| ATM | | | | | ✓ | ✓ | | | |
| Kinase | | | | | | | | | |
| PI4KA | | | | | | | ✓ | ✓ | ✓ |

In certain embodiments, compounds of the present invention include a warhead group characterized in that provided compounds covalently modify the Cys862 residue of PI3-kinase alpha, thereby irreversibly inhibiting PI3 kinase-alpha.

In some embodiments, compounds of the present invention include a warhead group characterized in that provided compounds covalently modify one or more of Cys862 of PI3K-alpha, Cys2243 of MTOR, Cys838 of PI3K-alpha, Cys869 of PI3K-gamma, Cys815 of PI3K-delta, Cys841 of PI3K-beta, Class 1A, Cys1119 of PI3K-beta, Class 2, Cys3683 of DNA-PK, Cys2770 of ATM-Kinase, Cys2753 of ATM-Kinase, Cys1840 of PI4KA, Cys1844 of PI4KA, or Cys 1797 of PI4KA.

A conserved cysteine was identified across PI3K family members. Specifically, Cys869 of PI3K gamma corresponds to Cys838 of PI3K alpha, Cys815 of PI3K delta, Cys841 of PI3K beta, Class1 and Cys1119 of PI3K beta, Class2. In certain embodiments, compounds of the present invention include a warhead group characterized in that provided compounds target each of Cys869 of PI3K gamma, Cys838 of PI3K alpha, Cys815 of PI3K delta, Cys841 of PI3K beta, Class1 and Cys1119 of PI3K beta, Class2, thereby irreversibly inhibit each of these kinases.

Thus, in some embodiments, the R¹ warhead group is characterized in that the -L-Y moiety, as defined and described below, is capable of covalently binding to a cysteine residue thereby irreversibly inhibiting the enzyme. In certain embodiments, the cysteine residue is the Cys862 residue of PI3 kinase alpha. In some embodiments, the cysteine residue is any of Cys862 of PI3K-alpha, Cys2243 of MTOR, Cys838 of PI3K-alpha, Cys869 of PI3K-gamma, Cys815 of PI3K-delta, Cys841 of PI3K-beta, Class 1A, Cys1119 of PI3K-beta, Class 2, Cys3683 of DNA-PK, Cys2770 of ATM-Kinase, Cys2753 of ATM-Kinase, Cys1840 of PI4KA, Cys1844 of PI4KA, or Cys1797 of PI4KA. In other embodiments, the cysteine residue is any of Cys869 of PI3K gamma, Cys838 of PI3K alpha, Cys815 of PI3K delta, Cys841 of PI3K beta, Class1 or Cys1119 of PI3K beta, Class2. One of ordinary skill in the art will recognize that a variety of warhead groups, as defined herein, are suitable for such covalent bonding. Such R¹ groups include, but are not limited to, those described herein and depicted in Table 4, infra.

In certain embodiments, the present invention provides a conjugate comprising one or more PI3 kinases having a cysteine residue, CysX, wherein the CysX is covalently, and irreversibly, bonded to an inhibitor, such that inhibition of the PI3 kinase is maintained, wherein CysX is selected from Cys862 of PI3K-alpha, Cys2243 of MTOR, Cys838 of PI3K-alpha, Cys869 of PI3K-gamma, Cys815 of PI3K-delta, Cys841 of PI3K-beta, Class 1A, Cys1119 of PI3K-beta, Class 2, Cys3683 of DNA-PK, Cys2770 of ATM-Kinase, Cys2753 of ATM-Kinase, Cys1840 of PI4KA, Cys1844 of PI4KA, or Cys1797 of PI4KA.

In certain embodiments, the present invention provides a conjugate of the formula C:

CysX-modifier-inhibitor moiety C

wherein:
the CysX is selected from Cys862 of PI3K-alpha, Cys2243 of MTOR, Cys838 of PI3K-alpha, Cys869 of PI3K-gamma, Cys815 of PI3K-delta, Cys841 of PI3K-beta, Class 1A, Cys1119 of PI3K-beta, Class 2, Cys3683 of DNA-PK, Cys2770 of ATM-Kinase, Cys2753 of ATM-Kinase, Cys1840 of PI4KA, Cys1844 of PI4KA, or Cys1797 of PI4KA;
the modifier is a bivalent group resulting from covalent bonding of a warhead group with the CysX of the PI3 kinase;
the warhead group is a functional group capable of covalently binding to CysX; and
the inhibitor moiety is a moiety that binds in the active site of the PI3 kinase.

In certain embodiments, the present invention provides a conjugate comprising PI3K-alpha having a cysteine residue, Cys862, wherein the Cys862 is covalently, and irreversibly, bonded to an inhibitor, such that inhibition of the PI3K-alpha is maintained.

In certain embodiments, the present invention provides a conjugate of the formula C-1:

Cys862-modifier-inhibitor moiety C-1

wherein:
the Cys862 is Cys862 of PI3K-alpha;
the modifier is a bivalent group resulting from covalent bonding of a warhead group with the Cys862 of the PI3K-alpha;
the warhead group is a functional group capable of covalently binding to Cys862; and
the inhibitor moiety is a moiety that binds in the active site of the PI3K-alpha.

In some embodiments, the present invention provides a conjugate comprising a PI3 kinase having a cysteine residue, wherein the cysteine is a conserved cysteine that is Cys869 of PI3K gamma, Cys838 of PI3K alpha, Cys815 of PI3K delta, Cys841 of PI3K beta, Class1 or Cys1119 of PI3K beta, Class2. In certain embodiments, the present invention provides a conjugate of the formula C-2:

CysX¹-modifier-inhibitor moiety C-2

wherein:
the CysX¹ is any one or more of Cys869 of PI3K gamma, Cys838 of PI3K alpha, Cys815 of PI3K delta, Cys841 of PI3K beta, Class 1 or Cys1119 of PI3K beta, Class 2;
the modifier is a bivalent group resulting from covalent bonding of a warhead group with the CysX¹ of the PI3 kinase;
the warhead group is a functional group capable of covalently binding to CysX¹; and
the inhibitor moiety is a moiety that binds in the active site of the PI3 kinase.

In certain embodiments, the inhibitor moiety of any of conjugates C, C-1, or C-2 is of formula I-i:

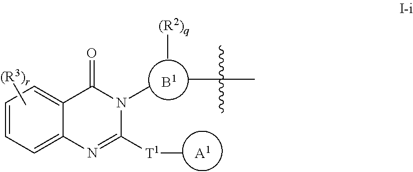

wherein the wavy bond indicates the point of attachment to CysX of conjugate C, Cys862 of conjugate C-1, or CysX¹ of conjugate C-2, via the modifier, and wherein each of the Ring A¹, Ring B¹, T¹, R², R³, q, and r groups of formula I-i is as defined for formula I below and described in classes and subclasses herein.

In other embodiments, the inhibitor moiety of any of conjugates C, C-1, or C-2 is of formula II-i, II-i-a, II-i-b, II-i-c, II-i-d, II-i-e, or II-i-f:

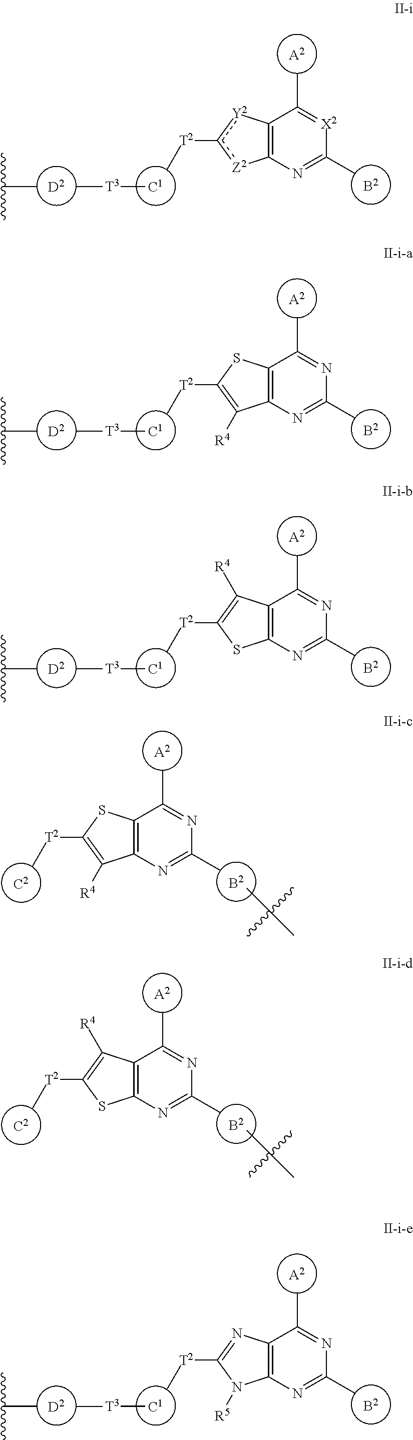

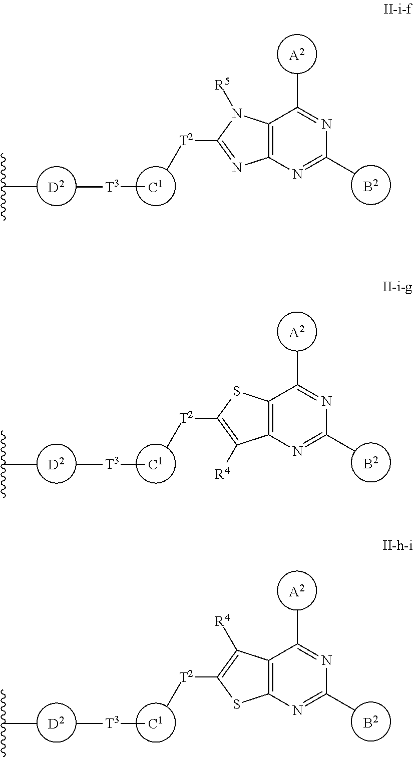

wherein the wavy bond indicates the point of attachment to CysX of conjugate C, Cys862 of conjugate C-1, or CysX¹ of conjugate C-2, and wherein each of the X², Y², Z², [symbol] , Ring A², Ring B², Ring C¹, Ring C², Ring D², T², T³, R⁴, and R⁵ groups of formula II-i-a, II-i-b, II-i-c, II-i-d, II-i-e, II-i-f, II-i-g, and II-i-h is as defined for formulae II, II-a, II-b, II-c, II-d, II-e, II-f, II-g, and II-h below and described in classes and subclasses herein.

In certain embodiments, compounds of formulae II-i-c and II-i-d are particularly selective for Cys869 of PI3K gamma. In certain embodiments, compounds of formulae II-1-c and II-i-d are pan-PI3K inhibitors.

In other embodiments, the inhibitor moiety of any of conjugates C, C-1, or C-2 is of formula III-i:

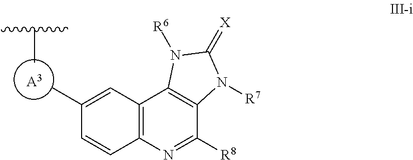

wherein the wavy bond indicates the point of attachment to CysX of conjugate C, Cys862 of conjugate C-1, or CysX¹ of conjugate C-2, and wherein each of the Ring A³, X, R⁶, R⁷, and R⁸ groups of formula III-i is as defined for formula III below and described in classes and subclasses herein.

In other embodiments, the inhibitor moiety of any of conjugates C, C-1, or C-2 is of formula IV-i:

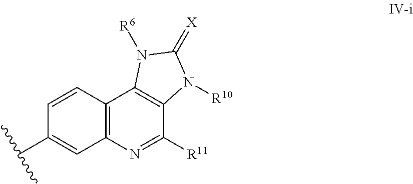

wherein the wavy bond indicates the point of attachment to CysX of conjugate C, Cys862 of conjugate C-1, or CysX¹ of conjugate C-2, and wherein each of the X, R⁹, R¹⁰, and R¹¹ groups of formula IV-i is as defined for formula IV below and described in classes and subclasses herein.

In other embodiments, the inhibitor moiety of any of conjugates C, C-1, or C-2 is of formula V-i-a or V-i-b:

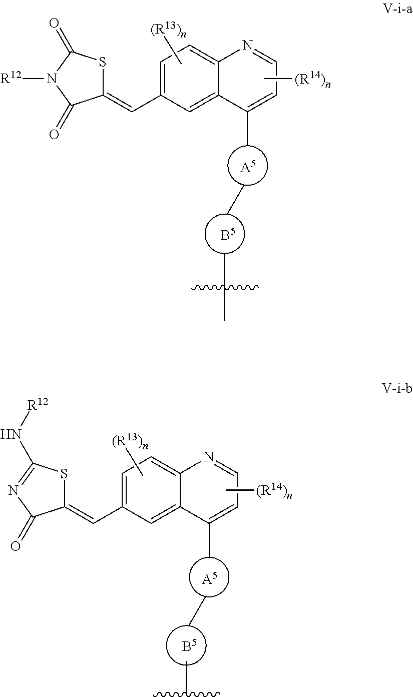

wherein the wavy bond indicates the point of attachment to CysX of conjugate C, Cys862 of conjugate C-1, or CysX¹ of conjugate C-2, and wherein each of the Ring A⁵, Ring B⁵, R¹², R¹³, R¹⁴, and n groups of formula V-i-a and V-i-b is as defined for formula V-a and V-b below and described in classes and subclasses herein.

In other embodiments, the inhibitor moiety of any of conjugates C, C-1, or C-2 is of formula VI-i-a or VI-i-b:

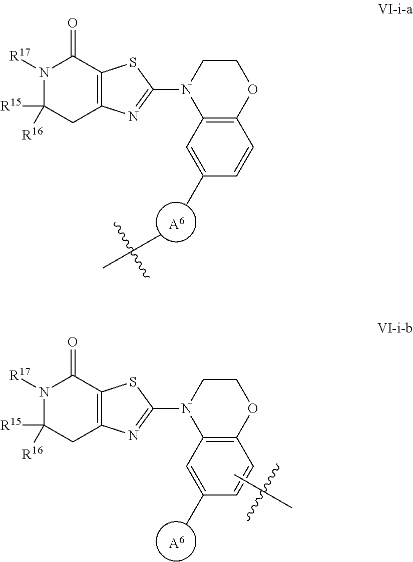

wherein the wavy bond indicates the point of attachment to CysX of conjugate C, Cys862 of conjugate C-1, or CysX¹ of conjugate C-2, and wherein each of the Ring A⁶, R¹⁵, R¹⁶, and R¹⁷ groups of formula VI-i-a and VI-i-b is as defined for formula VI-a and VI-b below and described in classes and subclasses herein.

In certain embodiments, the inhibitor moiety of any of conjugates C, C-1, or C-2 is of formula VII-i:

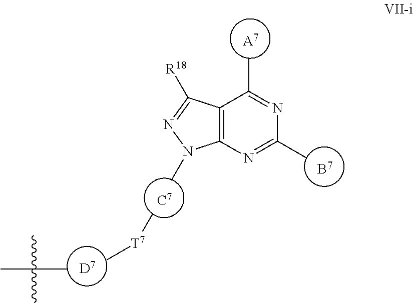

wherein the wavy bond indicates the point of attachment to CysX of conjugate C, Cys862 of conjugate C-1, or CysX¹ of conjugate C-2, and wherein each of the Ring A⁷, Ring B⁷, Ring C⁷, Ring D⁷, T⁷, and R¹⁸ groups of formula VII-i is as defined for formula VII below and described in classes and subclasses herein.

In certain embodiments, the inhibitor moiety of any of conjugates C, C-1, or C-2 is of formula VIII-i:

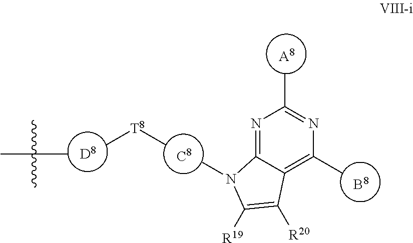

wherein the wavy bond indicates the point of attachment to CysX of conjugate C, Cys862 of conjugate C-1, or CysX¹ of conjugate C-2, and wherein each of the Ring A⁸, Ring B⁸, Ring C⁸, Ring D⁸, T⁸, R¹⁹, and R²⁰ groups of formula VIII-i is as defined for formula VIII below and described in classes and subclasses herein.

In certain embodiments, the inhibitor moiety of any of conjugates C, C-1, or C-2 is of formula IX-i:

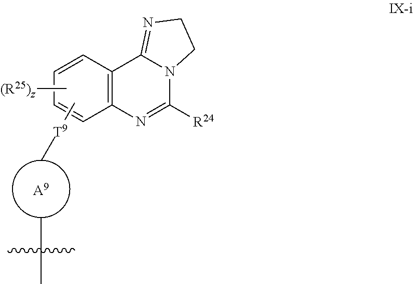

wherein the wavy bond indicates the point of attachment to CysX of conjugate C, Cys862 of conjugate C-1, or CysX¹ of conjugate C-2, and wherein each of the Ring A⁹, T⁹, R²⁴, R²⁵, and z groups of formula IX-i is as defined for formula IX below and described in classes and subclasses herein.

In certain embodiments, the inhibitor moiety of any of conjugates C, C-1, or C-2 is of formula X-i:

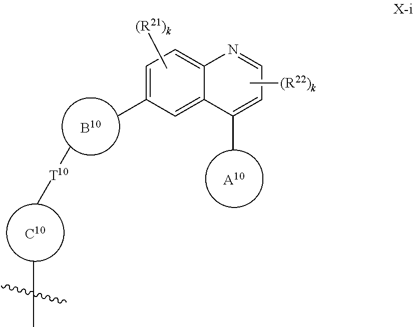

wherein the wavy bond indicates the point of attachment to CysX of conjugate C, Cys862 of conjugate C-1, or CysX¹ of conjugate C-2, and wherein each of the Ring A¹⁰, Ring B¹⁰, Ring C¹⁰, T¹⁰, R²¹, R²², and k groups of formula X-i is as defined for formula X below and described in classes and subclasses herein.

In certain embodiments, the inhibitor moiety of any of conjugates C, C-1, or C-2 is of formula XI-i:

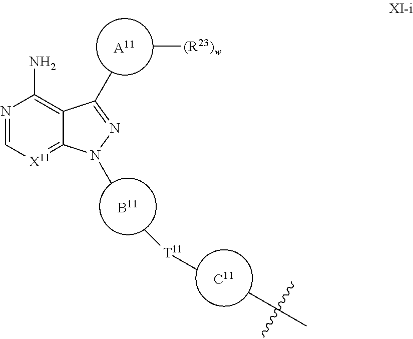

wherein the wavy bond indicates the point of attachment to CysX of conjugate C, Cys862 of conjugate C-1, or CysX¹ of conjugate C-2, and wherein each of the X¹¹, Ring A¹¹, Ring B¹¹, Ring C¹¹, T¹¹, R²³, and w groups of formula XI-i is as defined for formula XI below and described in classes and subclasses herein.

In certain embodiments, the inhibitor moiety of any of conjugates C, C-1, or C-2 is of formula XII-i:

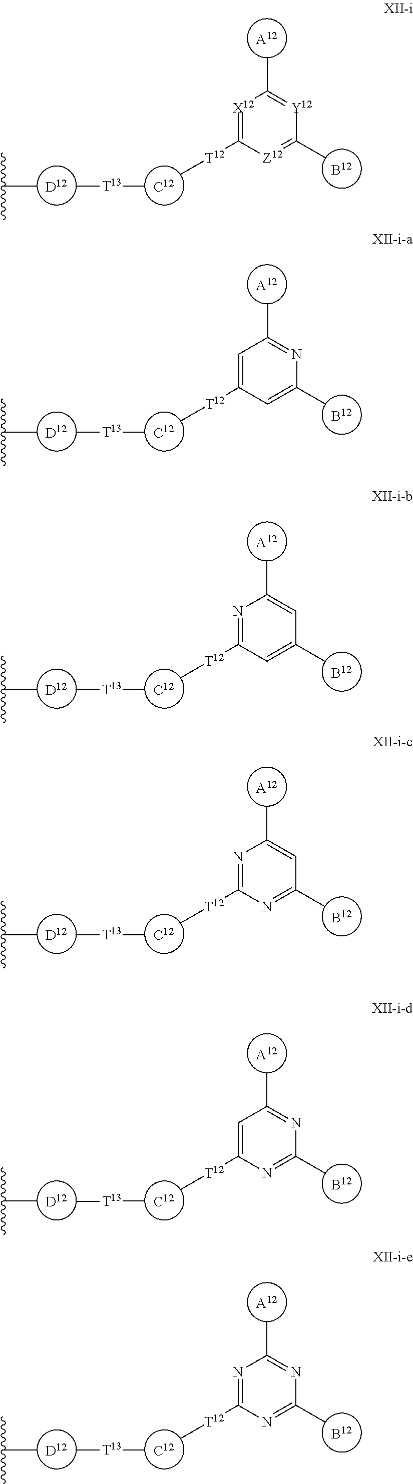

wherein the wavy bond indicates the point of attachment to CysX of conjugate C, Cys862 of conjugate C-1, or CysX¹ of conjugate C-2, and wherein each of the Ring A⁸, Ring B⁸, Ring C⁸, Ring D⁸, T⁸, R¹⁹, and R²⁰ groups of formulae XII-i, XII-i-a, XII-i-b, XII-i-c, XII-i-d, and XII-i-e is as defined for formula XII, XII-a, XII-b, XII-c, XII-d, and XII-e below and described in classes and subclasses herein.

In certain embodiments, the present invention provides a conjugate of any of formulae C-I-a, C-I-b, and C-I-c:

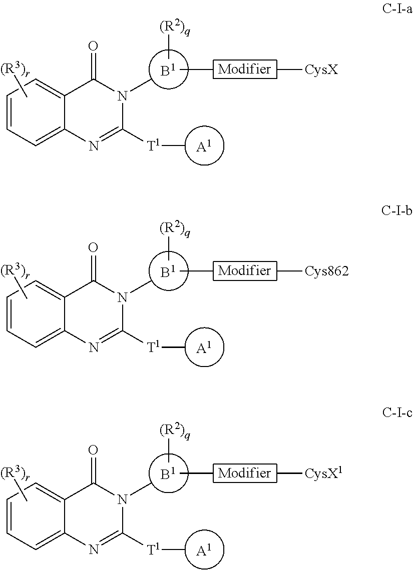

wherein each of the CysX, Cys862, and CysX¹ is as described herein and each of the Modifier, Ring A¹, Ring B¹, T¹, R², R³, q, and r groups of the conjugate is as defined for formula I below and described in classes and subclasses herein.

In some embodiments, the present invention provides a conjugate of any of formulae C-II-1, C-II-a-1, C-II-b-1, C-II-c-1, C-II-d-1, C-II-e-1, C-II-f-1, C-II-g-1, C-II-h-1, C-II-2, C-II-a-2, C-II-b-2, C-II-c-2, C-II-d-2, C-II-e-2, C-II-f-2, C-II-g-2, C-II-h-2, C-II-3, C-II-a-3, C-II-b-3, C-II-c-3, C-II-d-3, C-II-e-3, C-II-f-3, C-II-g-3, and C-II-h-3:

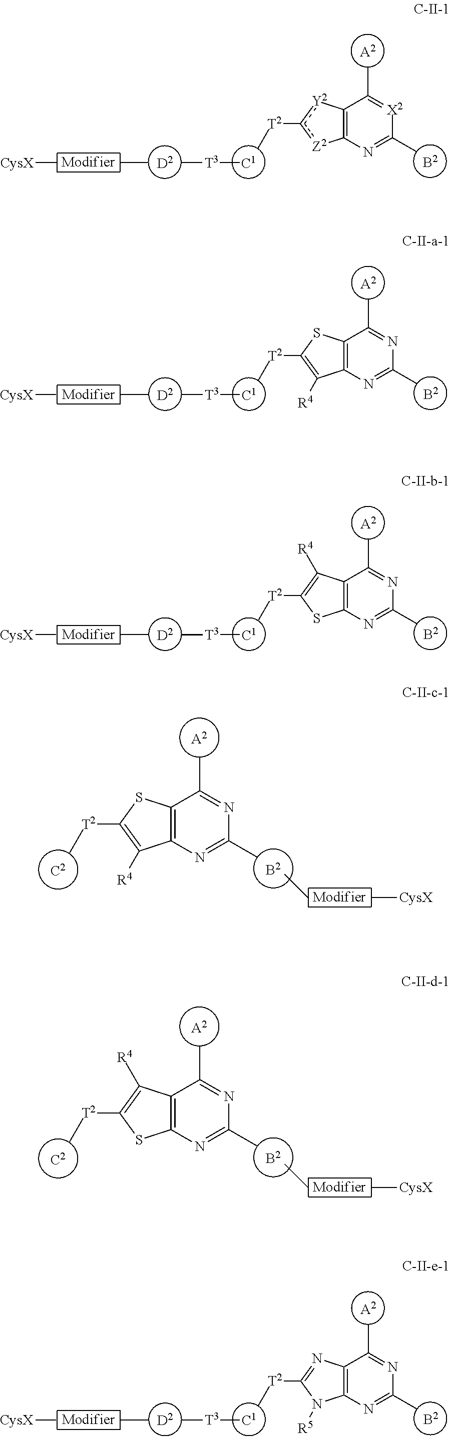

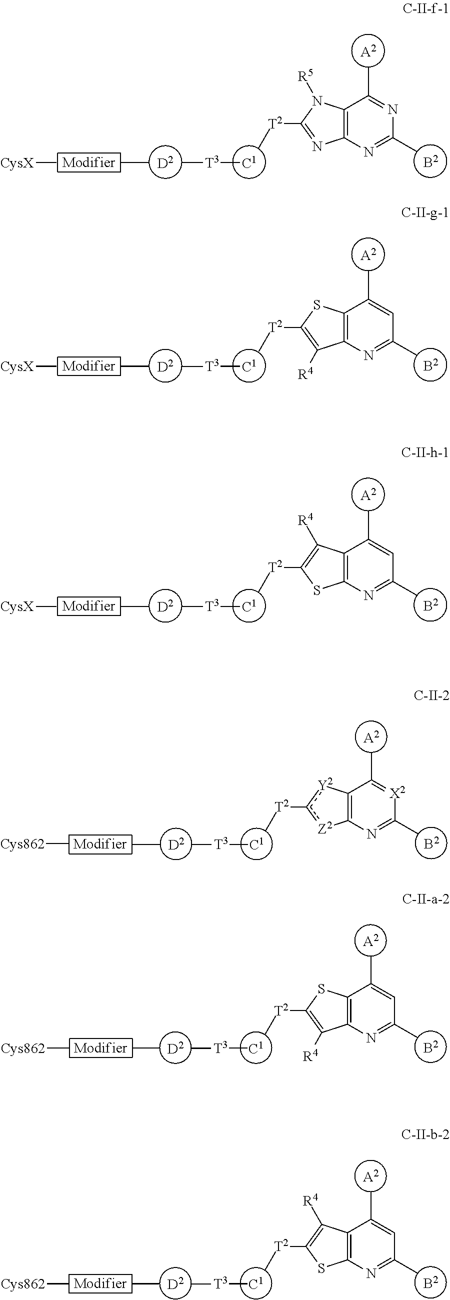

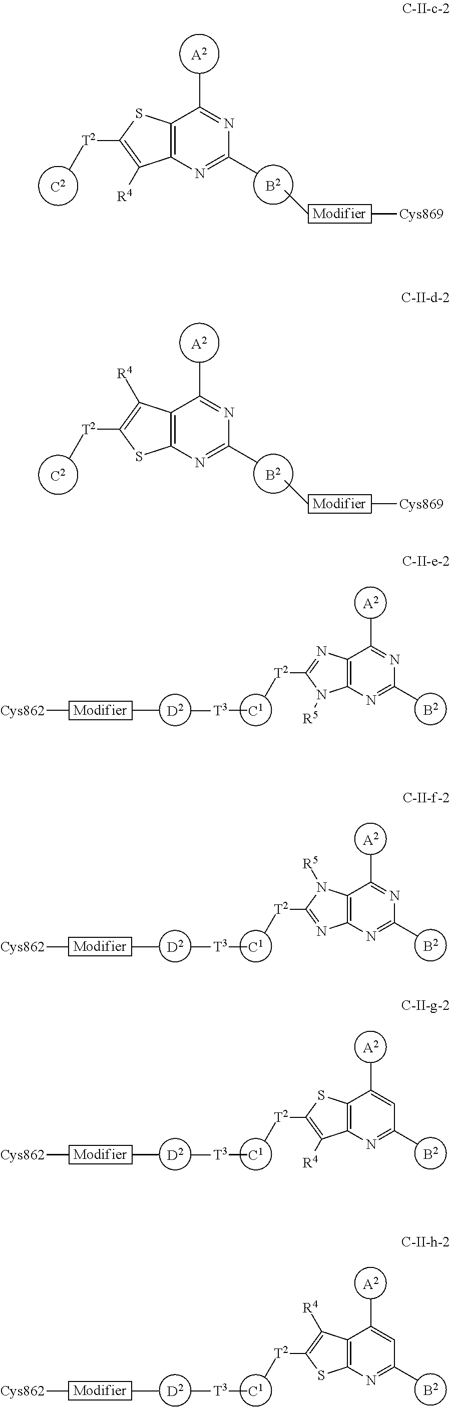

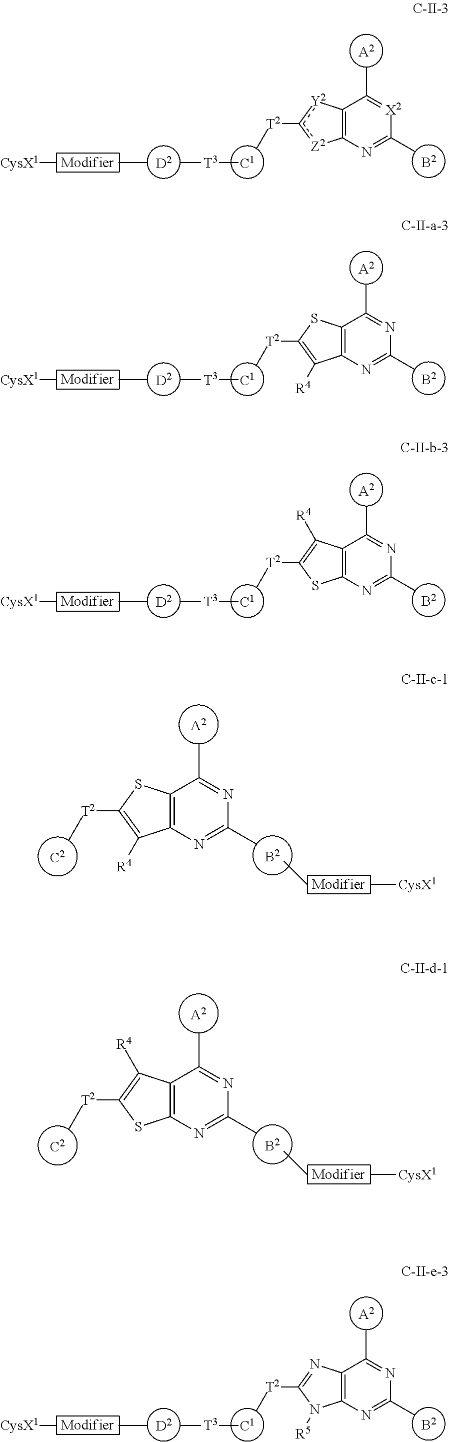

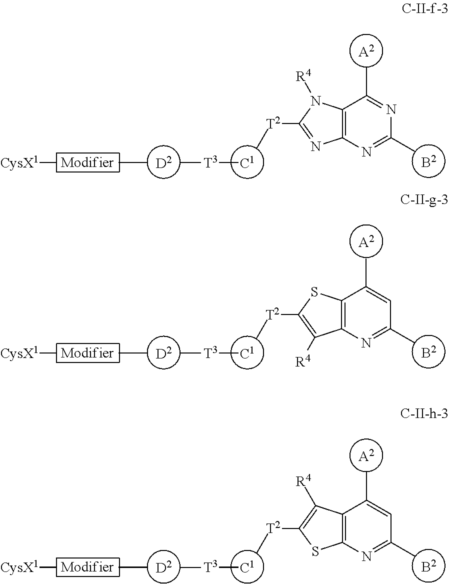

wherein each of the CysX, Cys862, Cys869, and CysX¹ is as described herein and each of the Modifier, X², Y², Z², Ring A², Ring B², Ring C¹, Ring C², Ring D², T², T³, R⁴, and R⁵ groups of the conjugate is as defined for formulae II-a, II-b, II-c, II-d, II-e, and II-f below and described in classes and subclasses herein.

In certain embodiments, the present invention provides a conjugate of any of formulae C-III-a, C-III-b, and C-III-c:

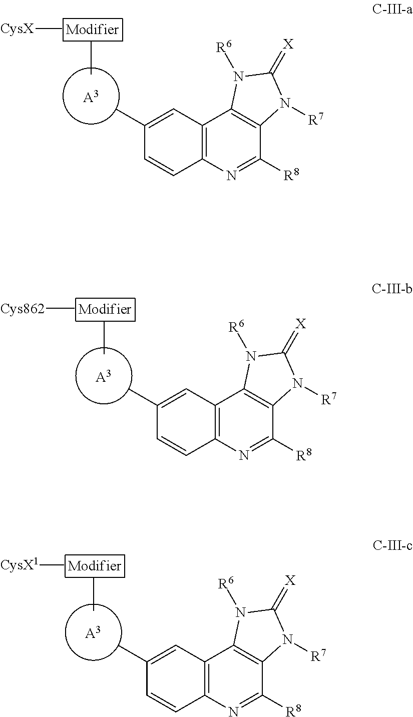

wherein each of the CysX, Cys862, and CysX¹ is as described herein and each of the Modifier, Ring A³, X, R⁶, R⁷, and R⁸ groups of the conjugate is as defined for formula III below and described in classes and subclasses herein.

In certain embodiments, the present invention provides a conjugate of any of formulae C-IV-a, C-IV-b, and C-IV-c:

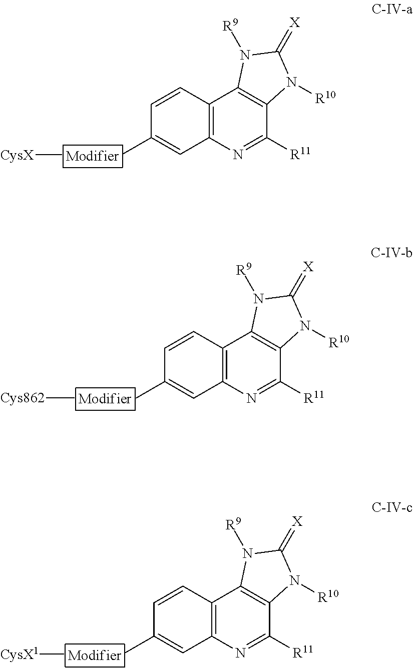

wherein each of the CysX, Cys862, and CysX¹ is as described herein and each of the Modifier, X, R⁹, R¹⁰, and R¹¹ groups of the conjugate is as defined for formula IV below and described in classes and subclasses herein.

In some embodiments, the present invention provides a conjugate of any of formulae C-V-a-1, C-V-b-1, C-V-a-2, C-V-b-2, C-V-a-3, and C-V-b-3:

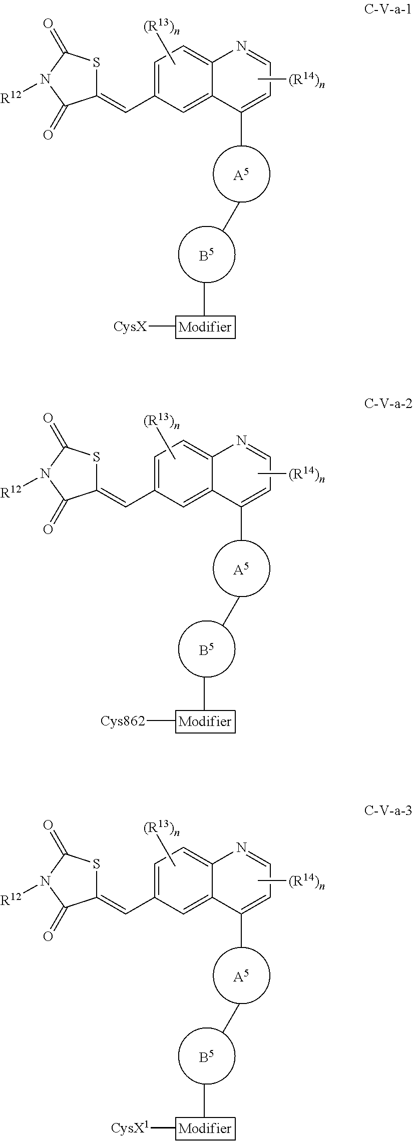

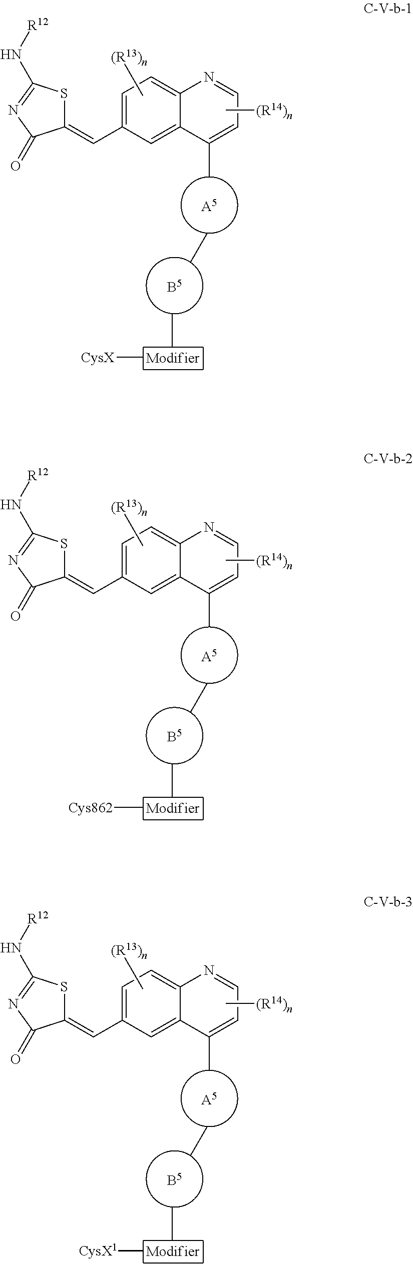

wherein each of the CysX, Cys862, and CysX¹ is as described herein and each of the Modifier, Ring A⁵, Ring B⁵, R¹², R¹³, R¹⁴, and n groups of the conjugate is as defined for formulae V-a and V-b below and described in classes and subclasses herein.

In some embodiments, the present invention provides a conjugate of any of formulae C-VI-a-1, C-VI-b-1, C-VI-a-2, C-VI-b-2, C-VI-a-3, and C-VI-b-3:

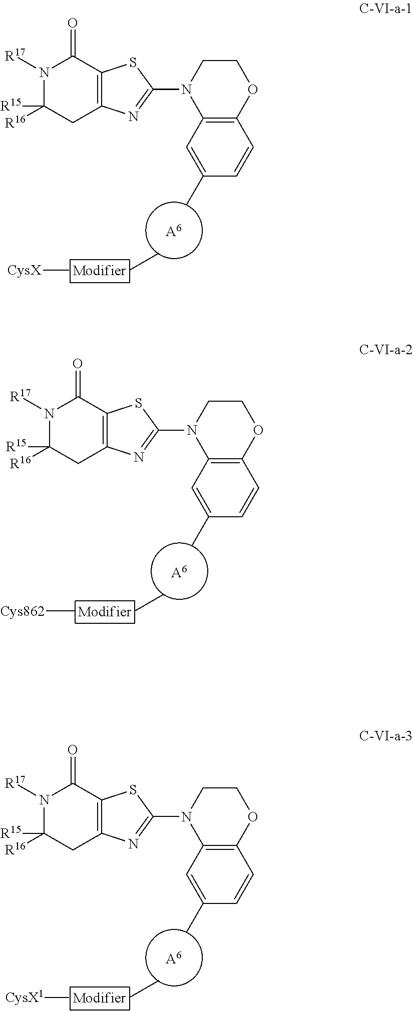

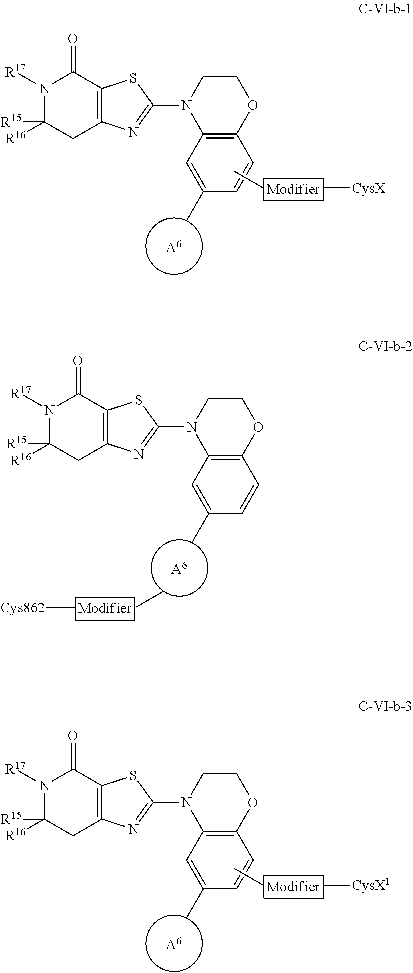

wherein each of the CysX, Cys862, and CysX¹ is as described herein and each of the Modifier, Ring A⁶, R¹⁵, R¹⁶, and R¹⁷ groups of the conjugate is as defined for formulae VI-a and VI-b below and described in classes and subclasses herein.

In certain embodiments, the present invention provides a conjugate of any of formulae C-VII-a, C-VII-b, and C-VII-c:

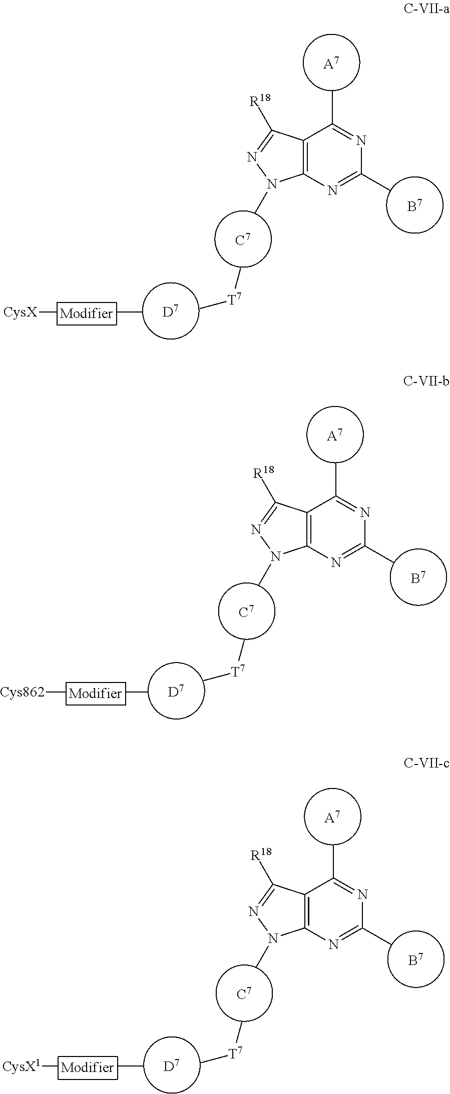

wherein each of the CysX, Cys862, and CysX¹ is as described herein and each of the Modifier, Ring A⁷, Ring B⁷, Ring C⁷, Ring D⁷, T⁷, and R¹⁸ groups of the conjugate is as defined for formula VII below and described in classes and subclasses herein.

In certain embodiments, the present invention provides a conjugate of any of formulae C-VIII-a, C-VIII-b, and C-VIII-c:

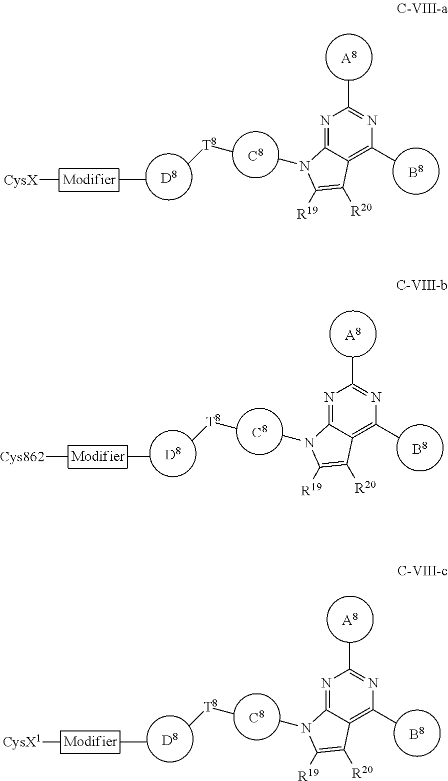

wherein each of the CysX, Cys862, and CysX¹ is as described herein and each of the Modifier, Ring A⁸, Ring B⁸, Ring C⁸, Ring D⁸, T⁸, R¹⁹, and R²⁰ groups of the conjugate is as defined for formula VIII below and described in classes and subclasses herein.

In certain embodiments, the present invention provides a conjugate of any of formulae C-IX-a, C-IX-b, and C-IX-c:

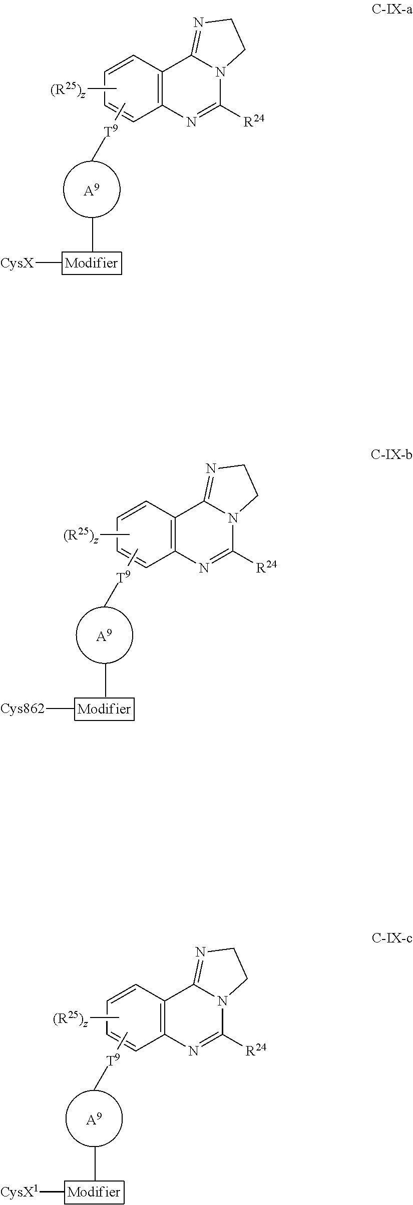

wherein each of the CysX, Cys862, and CysX¹ is as described herein and each of the Modifier, Ring A⁹, T⁹, R²⁴, R²⁵, and z groups of the conjugate is as defined for formula IX below and described in classes and subclasses herein.

In certain embodiments, the present invention provides a conjugate of any of formulae C-X-a, C-X-b, and C-X-c:

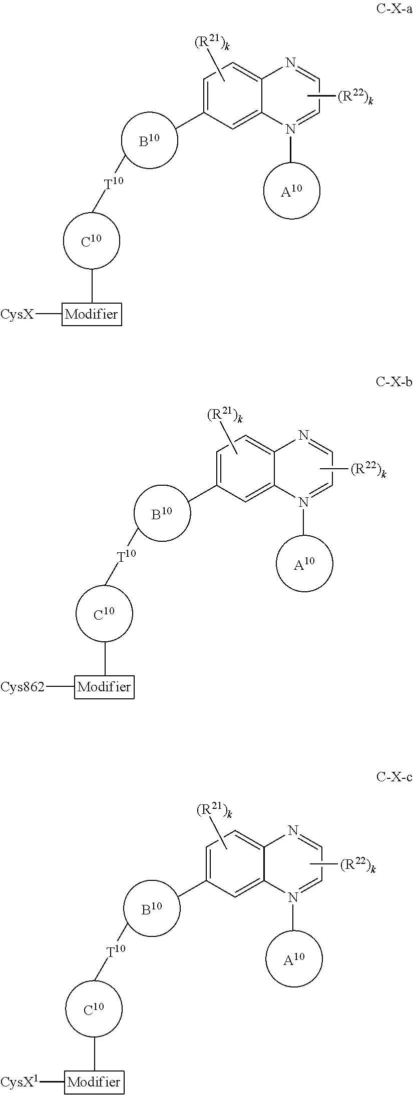

wherein each of the CysX, Cys862, and CysX¹ is as described herein and each of the Modifier, Ring A¹⁰, Ring B¹⁰, Ring C¹⁰, T¹⁰, R²¹, R²², and k groups of the conjugate is as defined for formula X below and described in classes and subclasses herein.

In certain embodiments, the present invention provides a conjugate of any of formulae C-XI-a, C-XI-b, and C-XI-c:

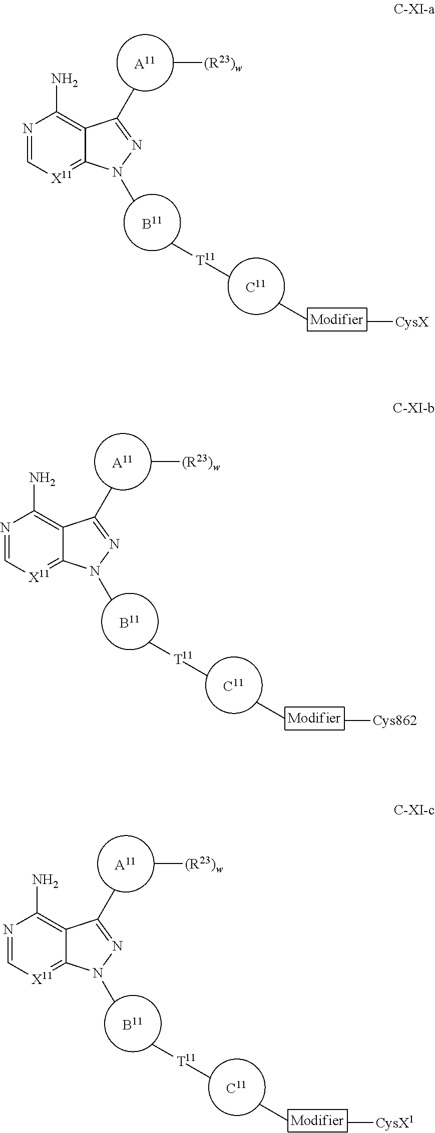

wherein each of the CysX, Cys862, and CysX¹ is as described herein and each of the Modifier, X¹¹, Ring A¹¹, Ring B¹¹, Ring C¹¹, T¹¹, R²³, and w groups of the conjugate is as defined for formula XI below and described in classes and subclasses herein.

In certain embodiments, the present invention provides a conjugate of any of formulae C-XII-1, C-XII-a-1, C-XII-b-1, C-XII-c-1, C-XII-d-1, C-XII-e-1, C-XII-2, C-XII-a-2, C-XII-b-2, C-XII-c-2, C-XII-d-2, C-XII-e-2, C-XII-3, C-XII-a-3, C-XII-b-3, C-XII-c-3, C-XII-d-3, and C-XII-e-3:

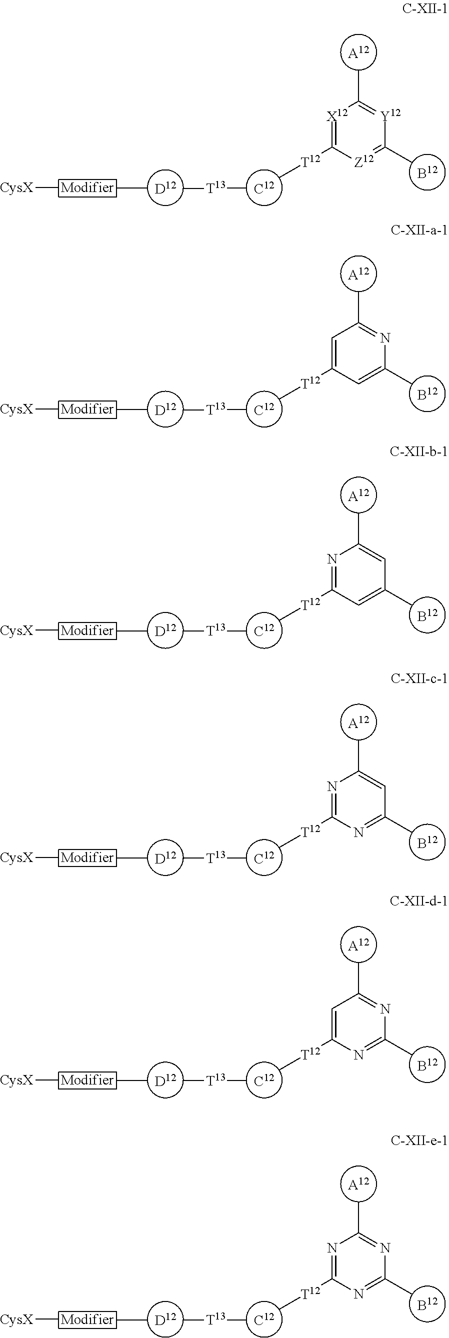

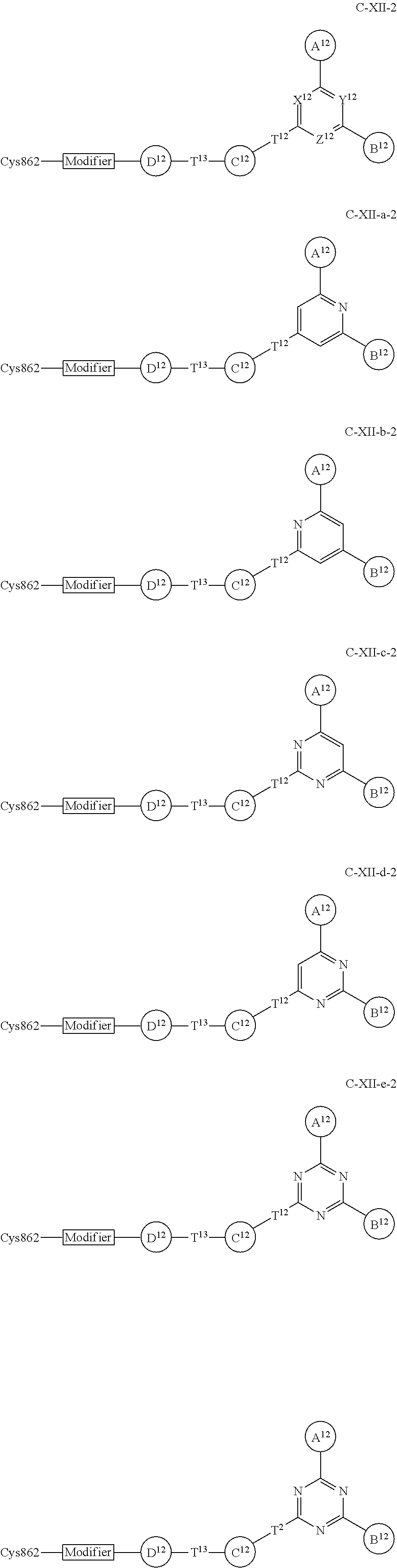

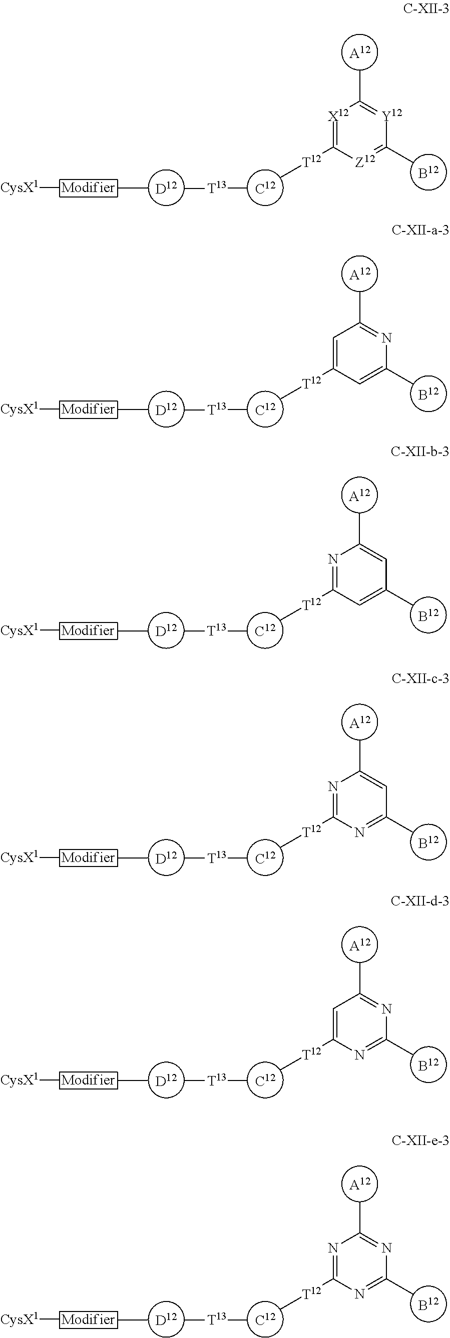

wherein each of the CysX, Cys862, and CysX¹ is as described herein and each of the Modifier, Ring A¹², Ring B¹², Ring C¹², Ring D¹², T¹², and T¹³ groups of the conjugate is as defined for formulae XII, XII-a, XII-b, XII-c, XII-d, and XII-e below and described in classes and subclasses herein.

In other embodiments, the modifier moiety of any of conjugate C, C-1, C-2, C-I-a, C-I-b, C-I-c, C-II-1, C-II-a-1, C-II-b-1, C-II-c-1, C-II-d-1, C-II-e-1, C-II-f-1, C-II-g-1, C-II-h-1, C-II-2, C-II-a-2, C-II-b-2, C-II-c-2, C-II-d-2, C-II-e-2, C-II-f-2, C-II-g-2, C-II-h-2, C-II-3, C-II-a-3, C-II-b-3, C-II-c-3, C-II-d-3, C-II-e-3, C-II-f-3, C-II-g-3, C-II-h-3, C-III-a, C-III-b, C-III-c, C-IV-a, C-IV-b, C-IV-c, C-V-a-1, C-V-b-1, C-V-a-2, C-V-b-2, C-V-a-3, C-V-b-3, C-VI-a-1, C-VI-b-1, C-VI-a-2, C-VI-b-2, C-VI-a-3, C-VI-b-3, C-VII-a, C-VII-b, C-VII-c, C-VIII-a, C-VIII-b, C-VIII-c, C-IX-a, C-IX-b, C-IX-c, C-X-a, C-X-b, C-X-c, C-XI-a, C-XI-b, C-XI-c, C-XII-1, C-XII-a-1, C-XII-b-1, C-XII-c-1, C-XII-d-1, C-XII-e-1, C-XII-2, C-XII-a-2, C-XII-b-2, C-XII-c-2, C-XII-d-2, C-XII-e-2, C-XII-3, C-XII-a-3, C-XII-b-3, C-XII-c-3, C-XII-d-3, and C-XII-e-3 is selected from those set forth in Table 2, below. Exemplary modifiers further include any bivalent group resulting from covalent bonding of a warhead moiety found in Table 3 or Table 4 with a cysteine of PI3 kinase. It will be understood that the exemplary modifiers below are shown as conjugated to the sulfhydryl of CysX.

TABLE 2
Exemplary Modifiers Conjugated to CysX:

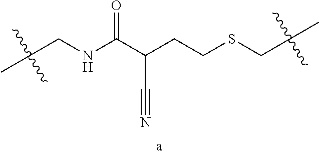

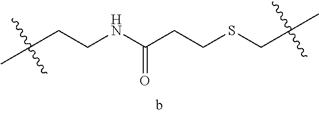

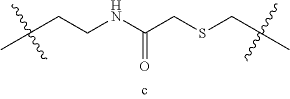

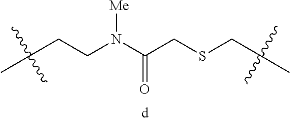

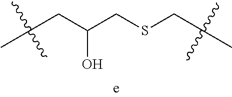

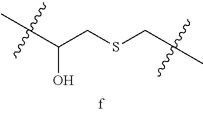

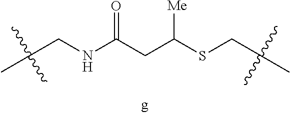

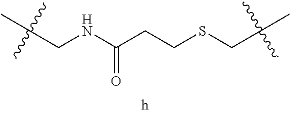

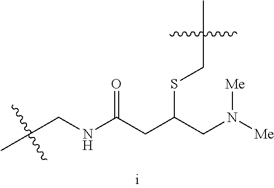

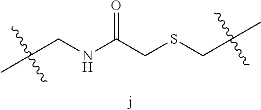

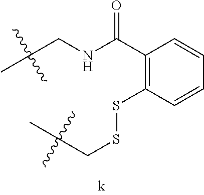

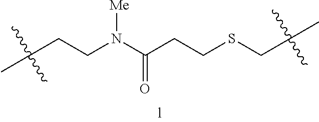

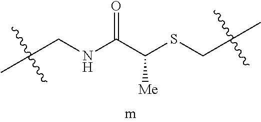

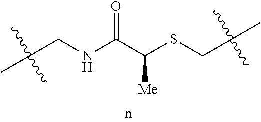

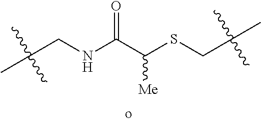

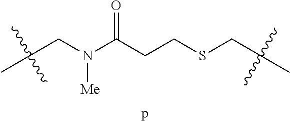

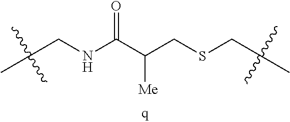

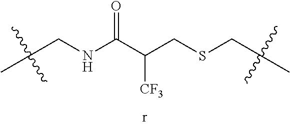

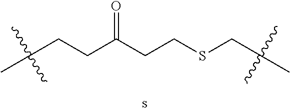

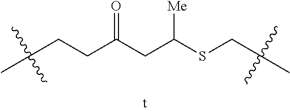

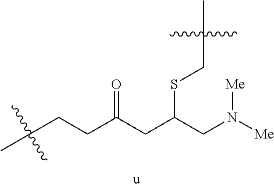

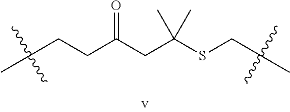

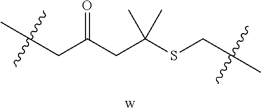

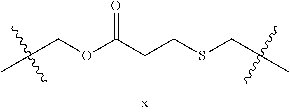

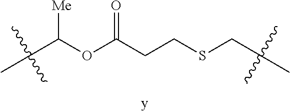

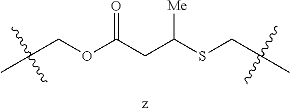

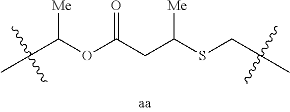

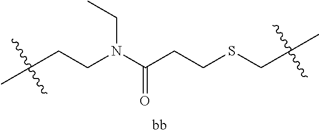

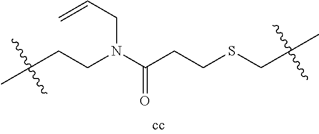

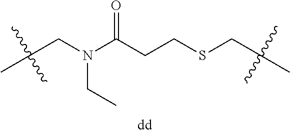

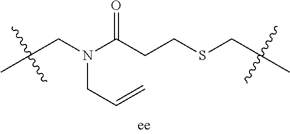

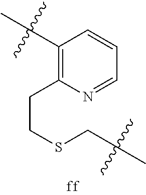

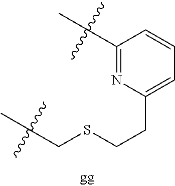

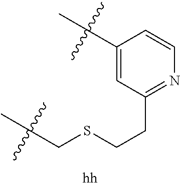

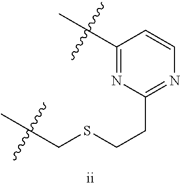

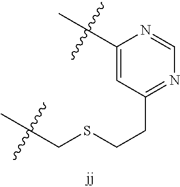

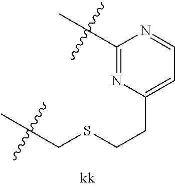

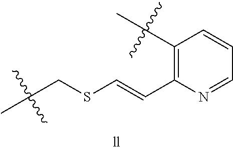

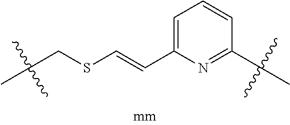

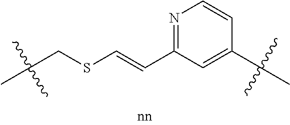

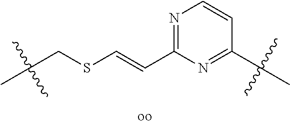

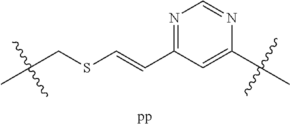

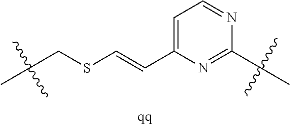

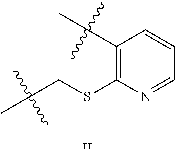

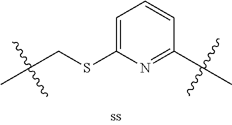

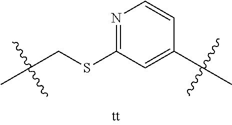

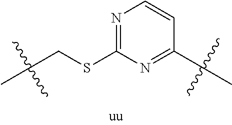

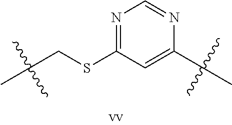

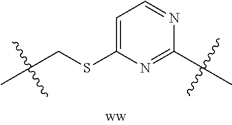

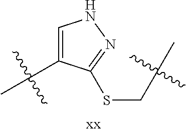

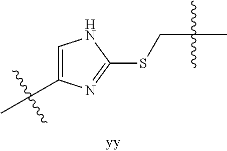

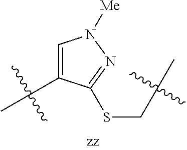

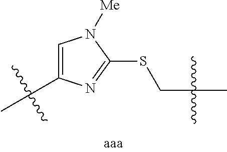

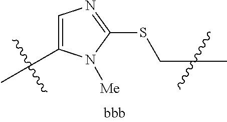

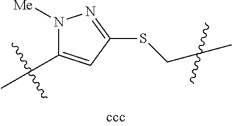

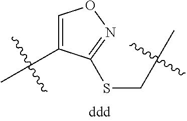

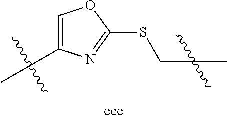

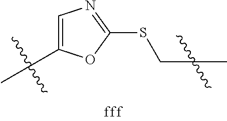

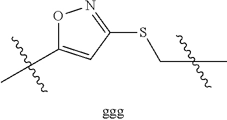

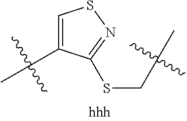

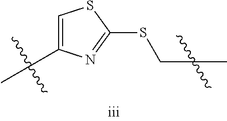

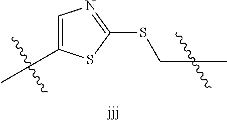

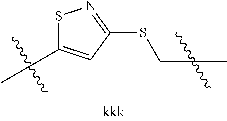

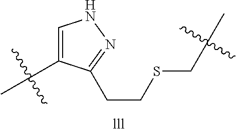

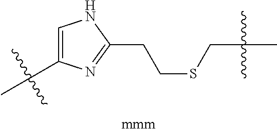

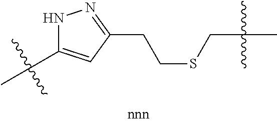

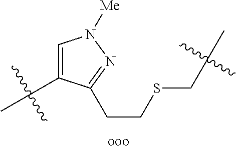

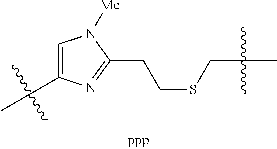

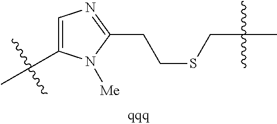

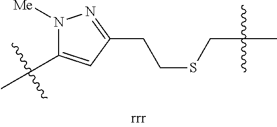

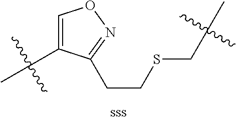

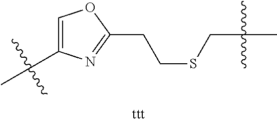

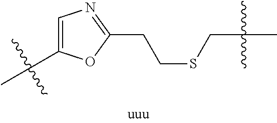

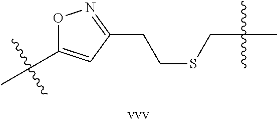

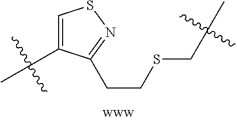

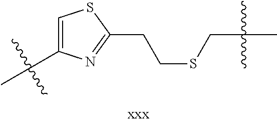

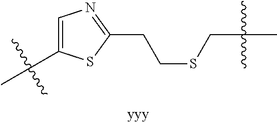

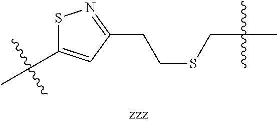

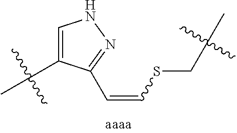

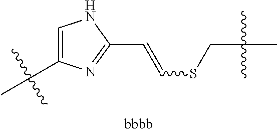

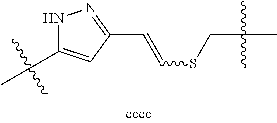

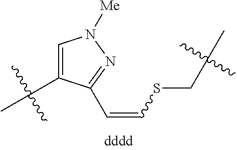

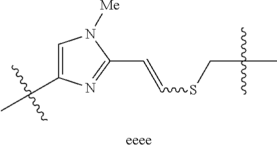

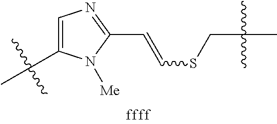

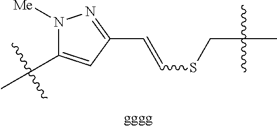

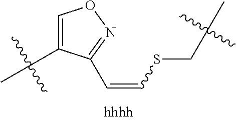

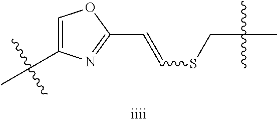

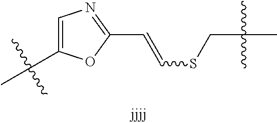

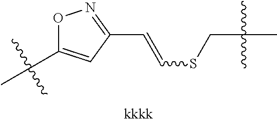

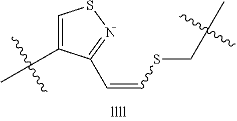

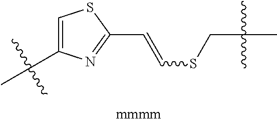

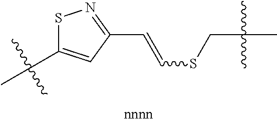

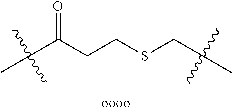

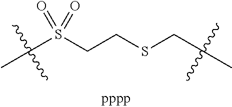

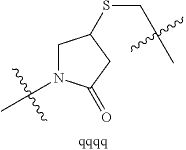

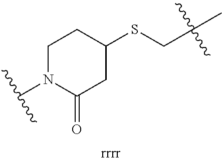

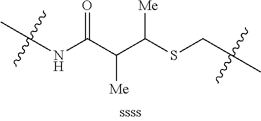

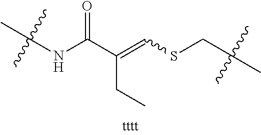

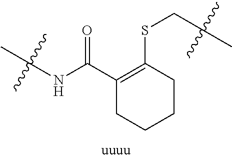

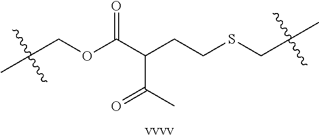

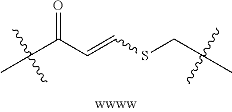

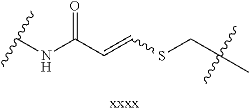

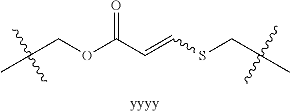

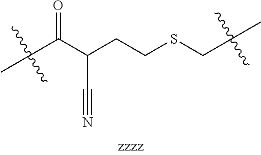

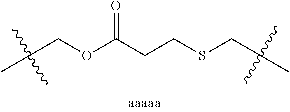

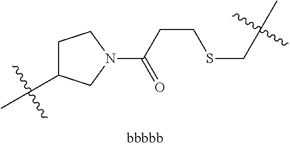

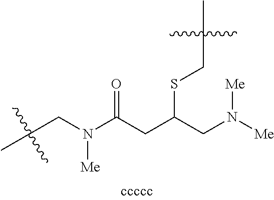

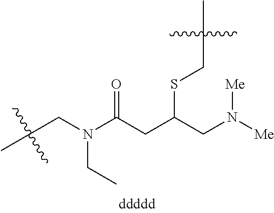

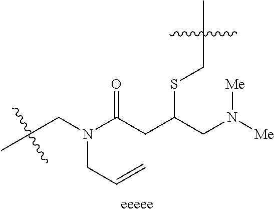

In certain embodiments, the present invention provides a compound of formula I:

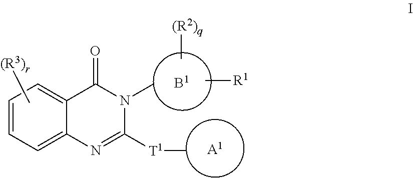

or a pharmaceutically acceptable salt thereof, wherein:
Ring A¹ is an optionally substituted group selected from an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
Ring B¹ is selected from phenyl, a 3-8 membered saturated or partially unsaturated carbocyclic ring, a 4-8 membered saturated or partially unsaturated heterocyclic ring having 1-2 heteroatoms independently selected from nitrogen, oxygen, or sulfur, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
R¹ is a warhead group;
T¹ is a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
   two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
q and r are each independently 0-4; and
each R² and R³ is independently R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, the Ring A¹ group of formula I is an optionally substituted group selected from an 8-10 membered bicyclic aryl ring or an 8-10 membered bicyclic heteroaryl ring having 1-4 heteroatoms independently selected from nitrogen, oxygen, or sulfur. In some embodiments, Ring A¹ is an optionally substituted 8-10 membered bicyclic heteroaryl ring having 2-4 nitrogen atoms. In one embodiment, Ring A¹ is 9H-purinyl.

In certain embodiments, the Ring B¹ group of formula I is an optionally substituted group selected from phenyl, a 3-8 membered saturated or partially unsaturated carbocyclic ring, or a 4-8 membered saturated or partially unsaturated heterocyclic ring having 1-2 heteroatoms independently selected from nitrogen, oxygen, or sulfur. In some embodiments, Ring B¹ is optionally substituted phenyl.

In certain embodiments, the T¹ group of formula I is a bivalent branched C₁₋₆ hydrocarbon chain wherein one or more methylene units of T¹ are replaced by —O—, —S—, or —N(R)—. In some embodiments, T is a bivalent straight C₁₋₆ hydrocarbon chain wherein one or more methylene units of T¹ are replaced by —O—, —S—, or —N(R)—.

In certain embodiments, the present invention provides a compound of formula II:

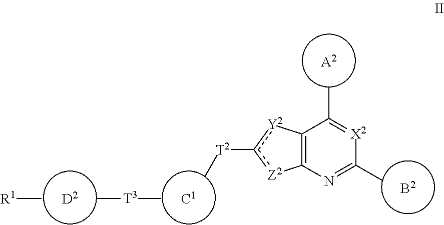

or a pharmaceutically acceptable salt thereof, wherein:
X² is CH or N;
Y² and Z² are independently CR⁴, C, NR⁵, N, O, or S, as valency permits;
[symbol] represents a single or double bond, as valency permits;
R¹ is a warhead group;
Ring A² is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R⁴ is —R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
R⁵ is —R, —SO₂R, —SOR, —C(O)R, —CO₂R, or —C(O)N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
   two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B² is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T² is a covalent bond or a bivalent straight or branched, saturated or unsaturated C₁₋₆ hydrocarbon chain wherein one or more methylene units of T² are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C¹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T³ is a covalent bond or a bivalent straight or branched, saturated or unsaturated C₁₋₆ hydrocarbon chain wherein one or more methylene units of T³ are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring D² is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

It will be understood by one of ordinary skill in the art that when Ring C¹ is absent, T³ is directly attached to T². It will be further understood that when Ring D² is absent, R¹ is directly attached to T³.

In certain embodiments, Y² is S and Z² is CR⁴. In certain embodiments, Y² is CR⁴ and Z² is S. In certain embodiments, Y² is N and Z² is NR⁵. In certain embodiments, Y² is NR⁵ and Z² is N.

In certain embodiments, the present invention provides a compound of formula II-a or II-b:

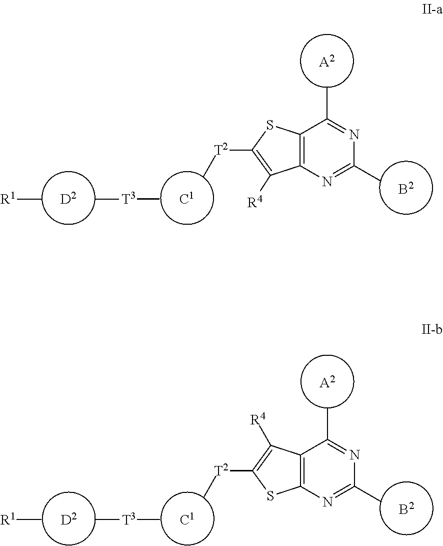

or a pharmaceutically acceptable salt thereof, wherein:
R¹ is a warhead group;
Ring A² is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R⁴ is —R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
   two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B² is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T² is a covalent bond or a bivalent straight or branched, saturated or unsaturated C₁₋₆ hydrocarbon chain wherein one or more methylene units of T² are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C¹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T³ is a covalent bond or a bivalent straight or branched, saturated or unsaturated C₁₋₆ hydrocarbon chain wherein one or more methylene units of T³ are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring D² is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

It will be understood by one of ordinary skill in the art that when Ring C¹ is absent, T³ is directly attached to T². It will be further understood that when Ring D² is absent, R¹ is directly attached to T³.

In certain embodiments, the Ring B² group of either of formula II-a or II-b is an optionally substituted 8-10 membered bicyclic heteroaryl ring having 1-4 heteroatoms independently selected from nitrogen, oxygen, or sulfur. In some embodiments, Ring B² is an optionally substituted 8-10 membered bicyclic heteroaryl ring having 2 nitrogen atoms. In some embodiments, Ring B² is 1H-indazolyl, benzimidazolyl, or indolyl. In certain embodiments, Ring B² is 1H-indazolyl. In certain embodiments, the Ring B² group is substituted or unsubstituted phenyl. In certain embodiments, Ring B² is substituted phenyl. In certain embodiments, Ring B² is phenol. In some embodiments, Ring B² is a 5-6 membered heteroaryl ring having 1-4 heteroatoms independently selected from nitrogen, oxygen, or sulfur. In some embodiments, Ring B² is an optionally substituted 5-6 membered heteroaryl ring having 1-2 nitrogen atoms. In certain embodiments, Ring B² is pyridyl. In certain embodiments, Ring B² is optionally substituted pyrimidinyl. In certain embodiments, Ring B² is

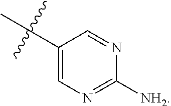

In certain embodiments, the Ring A² group of either of formula II-a or II-b is an optionally substituted 5-6 membered saturated or partially unsaturated heterocyclic ring having one or two heteroatoms independently selected from nitrogen, oxygen, or sulfur. In some embodiments, Ring A² is an optionally substituted 6-membered saturated or partially unsaturated heterocyclic ring having one or two heteroatoms independently selected from nitrogen, oxygen, or sulfur. In some embodiments, Ring A² is optionally substituted morpholinyl. In certain embodiments, Ring A² is unsubstituted morpholinyl. In some embodiments, Ring A² is optionally substituted tetrahydropyranyl. In certain embodiments, A² is:

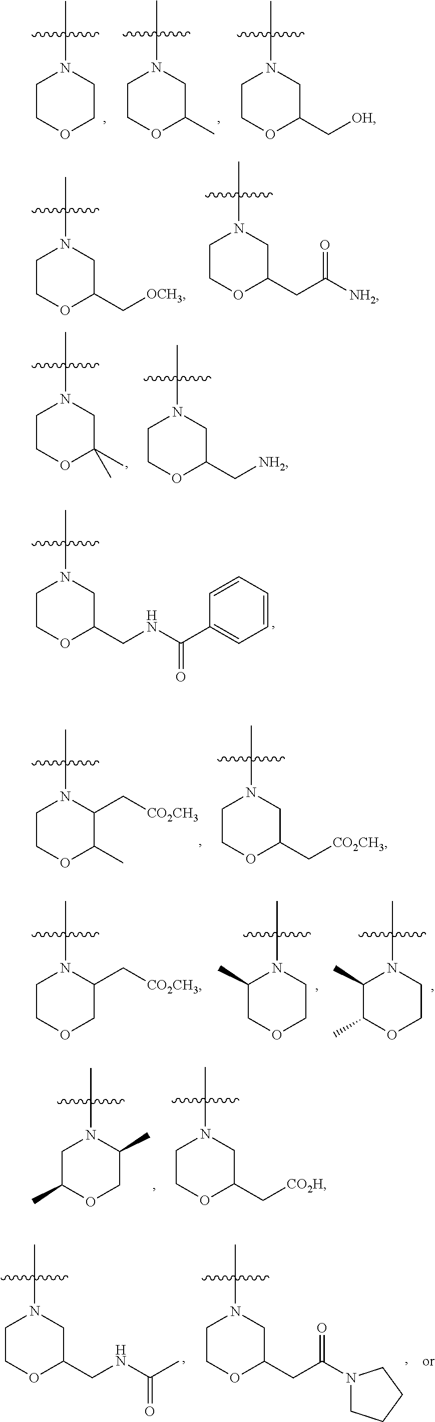

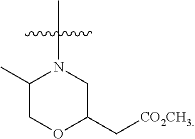

In certain embodiments, Ring A² is an optionally substituted ring 5-15 membered saturated or partially unsaturated bridged bicyclic heterocyclic ring having at least one nitrogen, at least one oxygen, and optionally 1-2 additional heteroatoms independently selected from nitrogen, oxygen, or sulfur. In certain embodiments, Ring A² is an optionally substituted ring 5-10 membered saturated or partially unsaturated bridged bicyclic heterocyclic ring having at least one nitrogen, at least one oxygen, and optionally 1-2 additional heteroatoms independently selected from nitrogen, oxygen, or sulfur. In certain embodiments, Ring A² is a bridged, bicyclic morpholino group. In certain embodiments, A² is an optionally substituted ring having the structure:

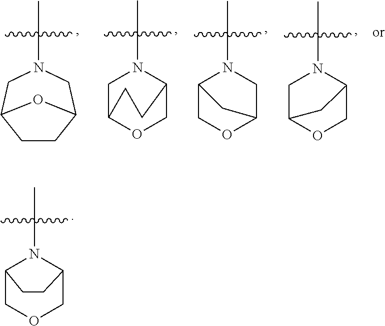

In certain embodiments, Ring A² is of the formula:

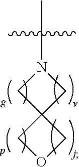

wherein:
v, j, p, and g are independently 1, 2, or 3.

In some embodiments, Ring A² is an optionally substituted bicyclic (fused or spiro-fused) ring selected from:

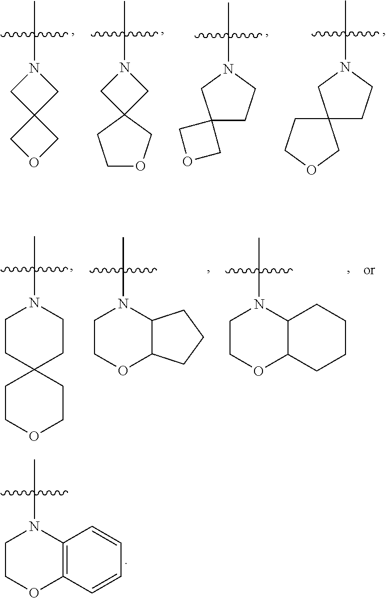

In certain embodiments, the T² group of either of formula II-a or II-b is a bivalent, straight, saturated C₁₋₆ hydrocarbon chain. In some embodiments, T² is a bivalent, straight, saturated C₁₋₃ hydrocarbon chain. In some embodiments, T² is —CH₂— or —CH₂CH₂—. In other embodiments, T² is —C(O)—. In certain embodiments, T² is —C≡C— or —CH₂C≡C—. In certain embodiments, T² is a covalent bond. In some embodiments, T² is a covalent bond, methylene, or a C₂₋₄ hydrocarbon chain wherein one methylene unit of T² is replaced by —C(O)NH—. In certain embodiments, T² is a C₃ hydrocarbon chain wherein one methylene unit of T² is replaced by —C(O)NH—.

In certain embodiments, the Ring C¹ group of either of formula II-a or II-b is an optionally substituted 6-membered saturated heterocyclic ring having one or two heteroatoms independently selected from nitrogen, oxygen, or sulfur. In some embodiments, Ring C¹ is a piperazinyl or piperidinyl ring. In some embodiments, Ring C¹ is an optionally substituted 6-membered partially unsaturated heterocyclic ring having one or two heteroatoms independently selected from nitrogen, oxygen, or sulfur. In certain embodiments, Ring C¹ is tetrahydropyridyl. In some embodiments, Ring C¹ is phenyl. In some embodiments, Ring C¹ is an optionally substituted 3-7 membered saturated or partially unsaturated carbocyclic ring. In certain embodiments, Ring C¹ is cyclohexyl. In certain embodiments, Ring C¹ is absent. In some embodiments, Ring C¹ is a 7-12 membered saturated or partially unsaturated bridged bicyclic ring having 0-4 heteroatoms independently selected from nitrogen, oxygen, or sulfur.

In certain embodiments, the T³ group of either of formula II-a or II-b is a bivalent, straight, saturated C₁₋₆ hydrocarbon chain. In some embodiments, T³ is a bivalent, straight, saturated C₁₋₃ hydrocarbon chain. In some embodiments, T³ is —CH₂— or —CH₂CH₂—. In certain embodiments, T³ is —C(O)—. In certain embodiments, T³ is a covalent bond.

In certain embodiments, the Ring D² group of either of formula II-a or II-b is an optionally substituted 6-membered saturated heterocyclic ring having one or two heteroatoms independently selected from nitrogen, oxygen, or sulfur. In some embodiments, Ring D² is a piperazinyl or piperidinyl ring. In some embodiments, Ring D² is an optionally substituted 6-membered partially unsaturated heterocyclic ring having one or two heteroatoms independently selected from nitrogen, oxygen, or sulfur. In certain embodiments, Ring D² is tetrahydropyridyl. In some embodiments, Ring D² is phenyl. In some embodiments, Ring D² is an optionally substituted 3-7 membered saturated or partially unsaturated carbocyclic ring. In certain embodiments, Ring D² is cyclohexyl. In certain embodiments, Ring D² is absent. In some embodiments, Ring D² is a 7-12 membered saturated or partially unsaturated bridged bicyclic ring having 0-4 heteroatoms independently selected from nitrogen, oxygen, or sulfur.

In some embodiments, a provided compound of formula II-a or II-b has one or more, more than one, or all of the features selected from:

a1) R¹ is selected from those embodiments described herein;
b1) Ring A² is selected from those embodiments described for formulae II-a and II-b, above;
c1) Ring B² is selected from those embodiments described for formulae II-a and II-b, above;
d1) T² is selected from those embodiments described for formulae II-a and II-b, above;
e1) Ring C¹ is selected from those embodiments described for formulae II-a and II-b, above;
f1) T³ is selected from those embodiments described for formulae II-a and II-b, above; and
g1) Ring D² is selected from those embodiments described for formulae II-a and II-b, above.

In some embodiments,

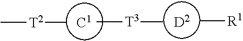

of formula II-a or II-b is

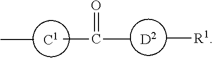

In some embodiments,

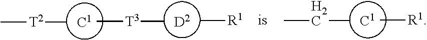

In some embodiments,

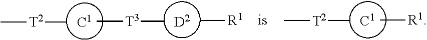

In some embodiments, a provided compound of formula II-a or II-b has one or more, more than one, or all of the features selected from:

a2) Ring A² is optionally substituted morpholinyl;
b2) Ring B² is an optionally substituted 8-10 membered bicyclic heteroaryl ring having 1-2 nitrogen atoms, optionally substituted phenyl, or an optionally substituted 5-6 membered heteroaryl ring having 1-2 nitrogen atoms;
c2)

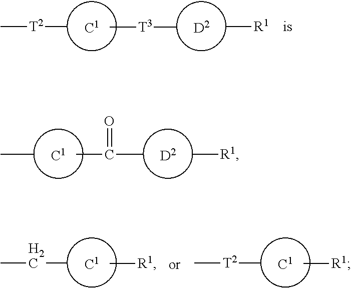

and
d2)

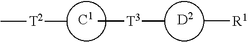

comprises a spacer group as defined herein having about 9 to about 11 atoms. In some embodiments, a provided compound of formula II-a or II-b has one or more, more than one, or all of the features selected from: a2), b2), c2), and d2) described above, and e2) R¹ is selected from those embodiments described herein.

In some embodiments, a provided compound of formula II-a or II-b has one or more, more than one, or all of the features selected from:

a3) Ring A² is optionally substituted morpholinyl;
b3) Ring B² is an optionally substituted group selected from indazolyl, aminopyrimidinyl, or phenol;
c3)

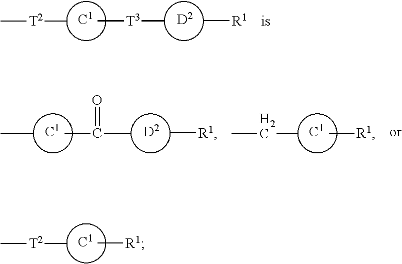

and
d3)

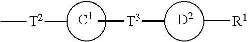

comprises a spacer group having about 9 to about 11 atoms. In some embodiments, a provided compound of formula II-a or II-b has one or more, more than one, or all of the features selected from: a3), b3), c3), and d3) described above, and e3) R¹ is selected from those embodiments described herein.

In some embodiments, a provided compound of formula II-a or II-b has one or more, more than one, or all of the features selected from:

a4) Ring A² is optionally substituted morpholinyl;
b4) Ring B² is an optionally substituted 8-10 membered bicyclic heteroaryl ring having 1-2 nitrogen atoms, optionally substituted phenyl, or an optionally substituted 5-6 membered heteroaryl ring having 1-2 nitrogen atoms;
c4) T² is a covalent bond, methylene, or a C₃₋₅ hydrocarbon chain wherein 2 methylene units of T² are replaced by —C(O)NH—;
d4) Ring C¹ is phenyl, or an optionally substituted 6-membered saturated, partially unsaturated, or aromatic heterocyclic ring having 1-2 nitrogens;
e4) T³ is a covalent bond, —C(O)—; and
f4) Ring D² is absent or phenyl.
In some embodiments, a provided compound of formula II-a or II-b has one or more, more than one, or all of the features selected from: a4), b4), c4), d4), e4), and f4) described above, and g4) R¹ is selected from those embodiments described herein.

In some embodiments, a provided compound of formula II-a or II-b has one or more, more than one, or all of the features selected from:

a5) Ring A² is optionally substituted morpholinyl;
b5) Ring B² is an optionally substituted group selected from indazolyl, phenol, or aminopyrimidine;
c5) T² is a covalent bond, methylene, or a C₄ hydrocarbon chain wherein 2 methylene units of T² are replaced by —C(O)NH—;
d5) Ring C¹ is phenyl, piperazinyl, piperidinyl, or tetrahydropyridyl;
e5) T³ is a covalent bond or —C(O)—; and
f5) Ring D² is absent or phenyl.
In some embodiments, a provided compound of formula II-a or II-b has one or more, more than one, or all of the features selected from: a5), b5), c5), d5), e5), and f5) described above, and g5) R¹ is selected from those embodiments described herein.

In certain embodiments, a provided compound of formula II-a or II-b has one of the following structures:

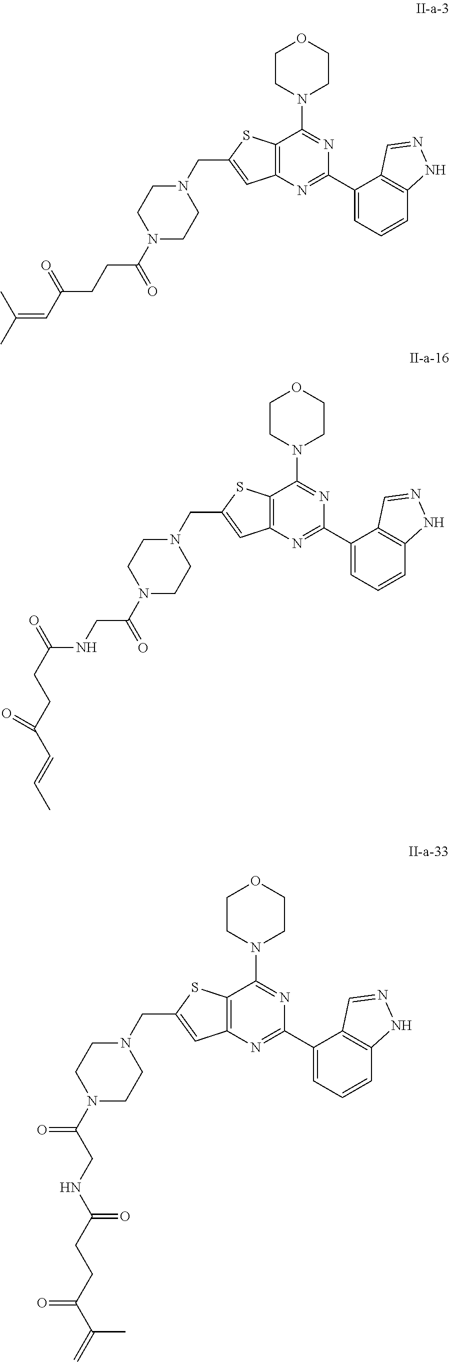

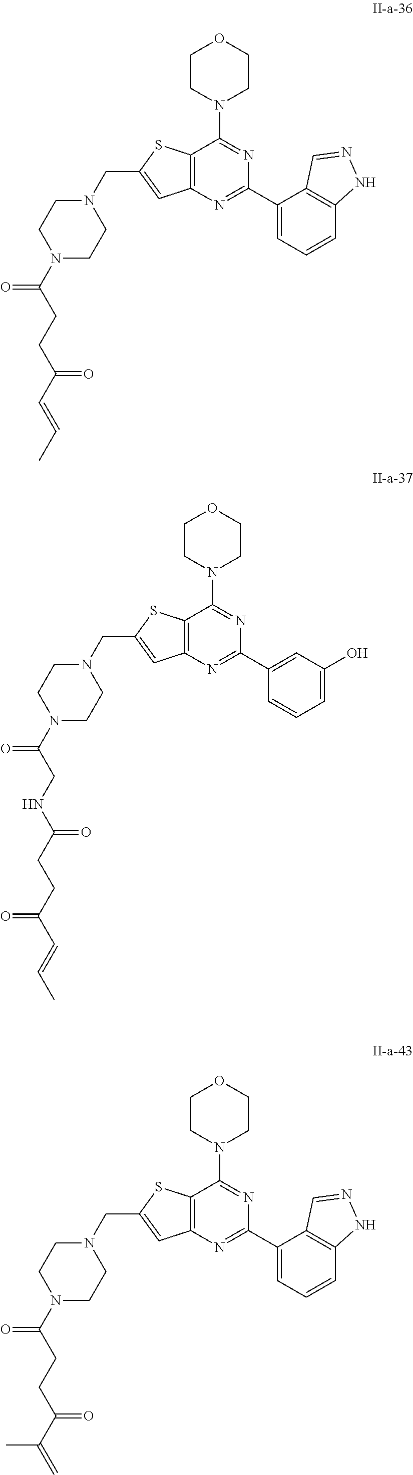

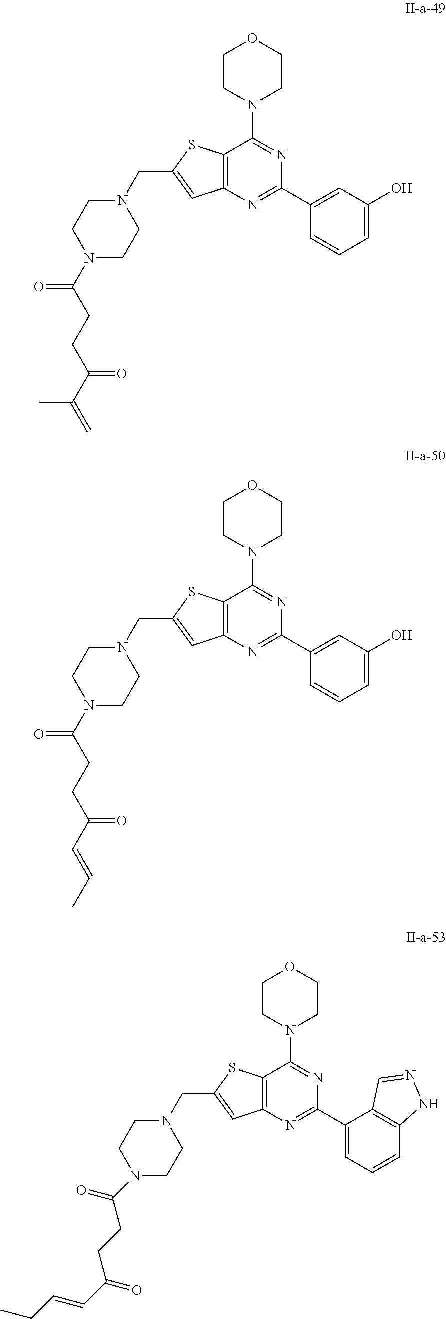

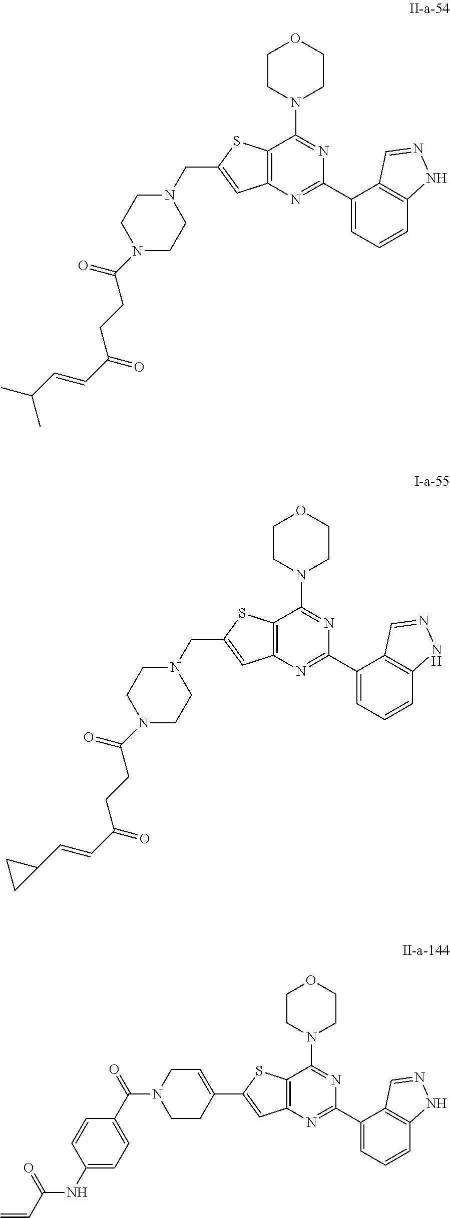

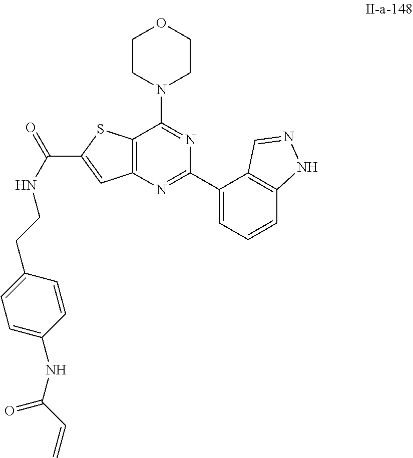

In certain embodiments, the present invention provides a compound of formula II-a-i or II-b-i:

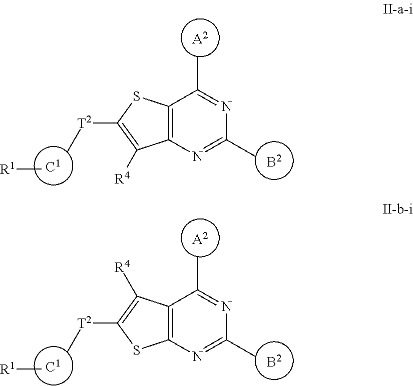

or a pharmaceutically acceptable salt thereof, wherein:
R¹, R⁴, R, Ring B², and T² are as defined above for formulae II-a and II-b and described in classes and subclasses herein;
Ring A² is an optionally substituted ring selected from a 4-8 membered saturated or partially unsaturated heterocyclic ring having one or two heteroatoms independently selected from nitrogen, oxygen, or sulfur, or a 5-10 membered saturated or partially unsaturated bridged bicyclic heterocyclic ring having at least one nitrogen, at least one oxygen, and optionally 1-2 additional heteroatoms independently selected from nitrogen, oxygen, or sulfur; and
Ring C¹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

In some embodiments, the present invention provides a compound of formula II-c or II-d:

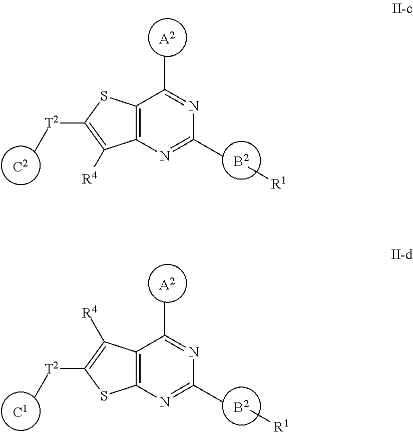

or a pharmaceutically acceptable salt thereof, wherein:
R¹ is a warhead group;
Ring A² is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R⁴ is R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
   two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B² is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T² is a covalent bond or a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring C² is hydrogen or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

In certain embodiments, the Ring B² group of either formula II-c or II-d is an optionally substituted 8-10 membered bicyclic heteroaryl ring having 1-4 heteroatoms independently selected from nitrogen, oxygen, or sulfur. In some embodiments, Ring B² is an optionally substituted 8-10 membered bicyclic heteroaryl ring having 2 nitrogen atoms. In some embodiments, Ring B² is 1H-indazolyl, benzimidazolyl, or indolyl. In certain embodiments, Ring B² is 1H-indazolyl. In certain embodiments, the Ring B² group is substituted or unsubstituted phenyl. In certain embodiments, Ring B² is substituted phenyl. In certain embodiments, Ring B² is phenol. In some embodiments, Ring B² is a 5-6 membered heteroaryl ring having 1-4 heteroatoms independently selected from nitrogen, oxygen, or sulfur. In some embodiments, Ring B² is an optionally substituted 5-6 membered heteroaryl ring having 1-2 nitrogen atoms. In certain embodiments, Ring B² is pyridyl. In certain embodiments, Ring B² is optionally substituted pyrimidinyl. In certain embodiments, Ring B² is

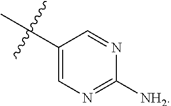

In certain embodiments, the Ring A² group of either of formula II-c or II-d is an optionally substituted 5-6 membered saturated or partially unsaturated heterocyclic ring having one or two heteroatoms independently selected from nitrogen, oxygen, or sulfur. In some embodiments, Ring A² is an optionally substituted 6-membered saturated or partially unsaturated heterocyclic ring having one or two heteroatoms independently selected from nitrogen, oxygen, or sulfur. In some embodiments, Ring A² is optionally substituted morpholinyl. In certain embodiments, Ring A² is unsubstituted morpholinyl. In some embodiments, Ring A² is optionally substituted tetrahydropyranyl. In certain embodiments, A² is:

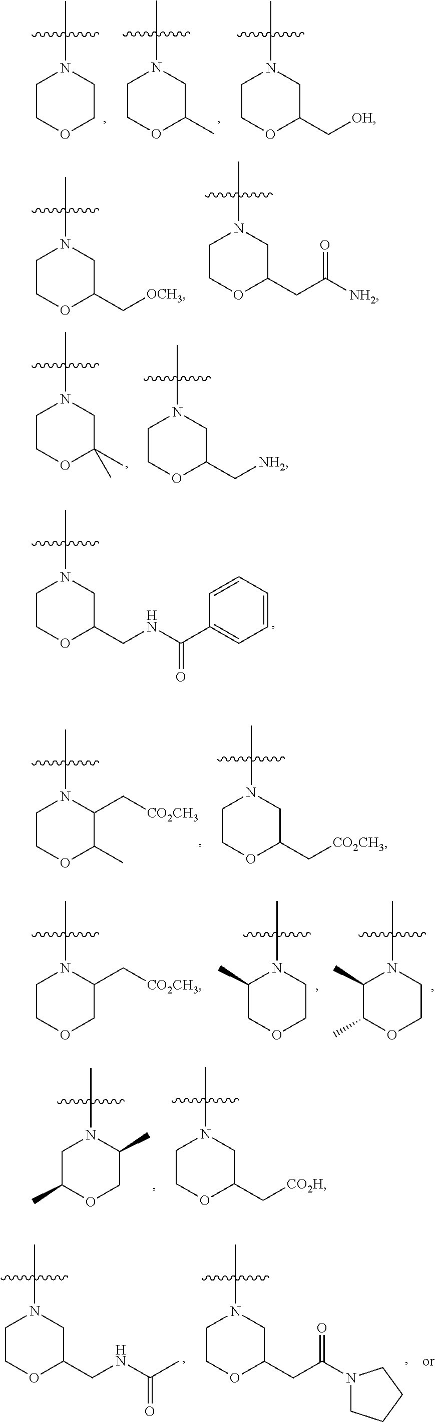

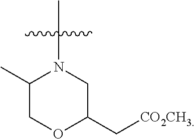

In certain embodiments, Ring A² is an optionally substituted ring 5-15 membered saturated or partially unsaturated bridged bicyclic heterocyclic ring having at least one nitrogen, at least one oxygen, and optionally 1-2 additional heteroatoms independently selected from nitrogen, oxygen, or sulfur. In certain embodiments, Ring A² is an optionally substituted ring 5-10 membered saturated or partially unsaturated bridged bicyclic heterocyclic ring having at least one nitrogen, at least one oxygen, and optionally 1-2 additional heteroatoms independently selected from nitrogen, oxygen, or sulfur. In certain embodiments, Ring A² is a bridged, bicyclic morpholino group. In certain embodiments, A² is an optionally substituted ring having the structure:

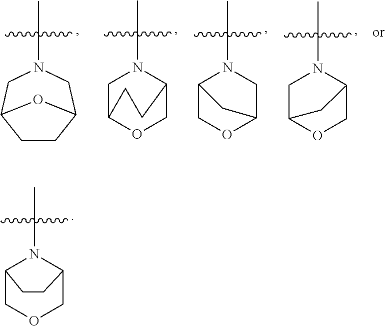

In certain embodiments, Ring A² is of the formula:

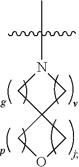

wherein:
v, j, p, and g are independently 1, 2, or 3.

In some embodiments, Ring A² is an optionally substituted bicyclic (fused or spiro-fused) ring selected from:

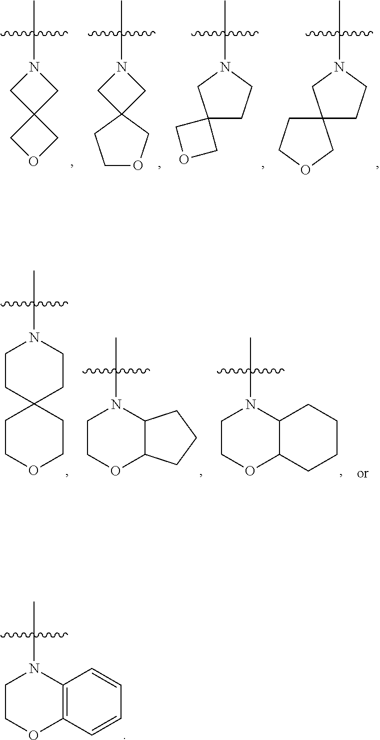

In certain embodiments, the T² group of either of formula II-c or II-d is a bivalent, straight, saturated C₁₋₆ hydrocarbon chain. In some embodiments, T² is a bivalent, straight, saturated C₁₋₃ hydrocarbon chain. In some embodiments, T² is —CH₂—. In certain embodiments, T² is a covalent bond.

In certain embodiments, the Ring C² group of either of formula II-c or II-d is an optionally substituted 6-membered saturated heterocyclic ring having one or two heteroatoms independently selected from nitrogen, oxygen, or sulfur. In some embodiments, Ring C² is a piperazinyl or piperidinyl ring. In some embodiments, Ring C² is an optionally substituted 6-membered partially unsaturated heterocyclic ring having one or two heteroatoms independently selected from nitrogen, oxygen, or sulfur. In certain embodiments, Ring C² is tetrahydropyridinyl. In some embodiments, Ring C² is phenyl. In some embodiments, Ring C² is an optionally substituted 3-7 membered saturated or partially unsaturated carbocyclic ring. In certain embodiments, Ring C² is cyclohexyl. In certain embodiments, Ring C² is hydrogen. In some embodiments, T² is a covalent bond and Ring C² is hydrogen. In some embodiments, Ring C² is a 7-12 membered saturated or partially unsaturated bridged bicyclic ring having 0-4 heteroatoms independently selected from nitrogen, oxygen, or sulfur.

In certain embodiments, the present invention provides a compound of formula II-e or II-f:

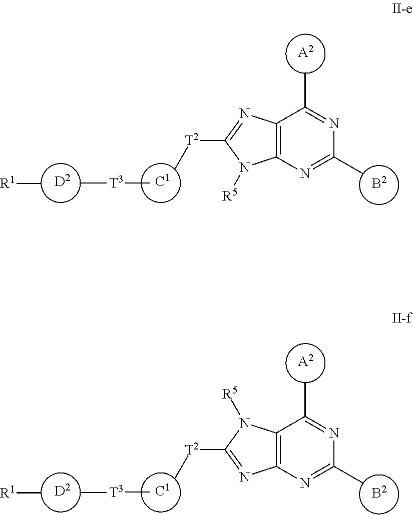

or a pharmaceutically acceptable salt thereof, wherein:
R¹ is a warhead group;
Ring A² is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R⁵ is R, —SO₂R, —SOR, —C(O)R, —CO₂R, or —C(O)N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
   two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B² is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T² is a covalent bond or a bivalent straight or branched, saturated or unsaturated C₁₋₆ hydrocarbon chain wherein one or more methylene units of T² are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C¹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T³ is a covalent bond or a bivalent straight or branched, saturated or unsaturated C₁₋₆ hydrocarbon chain wherein one or more methylene units of T³ are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring D² is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

It will be understood by one of ordinary skill in the art that when Ring C¹ of formula II-e or II-f is absent, T³ is directly attached to T². It will be further understood that when Ring D² is absent, R¹ is directly attached to T³.

In certain embodiments, the Ring B² group of either of formula II-e or II-f is an optionally substituted 8-10 membered bicyclic heteroaryl ring having 1-4 heteroatoms independently selected from nitrogen, oxygen, or sulfur. In some embodiments, Ring B² is an optionally substituted 8-10 membered bicyclic heteroaryl ring having 2 nitrogen atoms. In some embodiments, Ring B² is 1H-indazolyl, benzimidazolyl, or indolyl. In certain embodiments, Ring B² is 1H-indazolyl. In certain embodiments, the Ring B² group is substituted or unsubstituted phenyl. In certain embodiments, Ring B² is substituted phenyl. In certain embodiments, Ring B² is phenol. In some embodiments, Ring B² is a 5-6 membered heteroaryl ring having 1-4 heteroatoms independently selected from nitrogen, oxygen, or sulfur. In some embodiments, Ring B² is an optionally substituted 5-6 membered heteroaryl ring having 1-2 nitrogen atoms. In certain embodiments, Ring B² is pyridyl. In certain embodiments, Ring B² is optionally substituted pyrimidinyl. In certain embodiments, Ring B² is

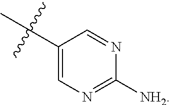

In certain embodiments, the Ring A² group of either of formula II-e or II-f is an optionally substituted 5-6 membered saturated or partially unsaturated heterocyclic ring having one or two heteroatoms independently selected from nitrogen, oxygen, or sulfur. In some embodiments, Ring A² is an optionally substituted 6-membered saturated or partially unsaturated heterocyclic ring having one or two heteroatoms independently selected from nitrogen, oxygen, or sulfur. In some embodiments, Ring A² is optionally substituted morpholinyl. In certain embodiments, Ring A² is unsubstituted morpholinyl. In some embodiments, Ring A² is optionally substituted tetrahydropyranyl. In certain embodiments, A² is:

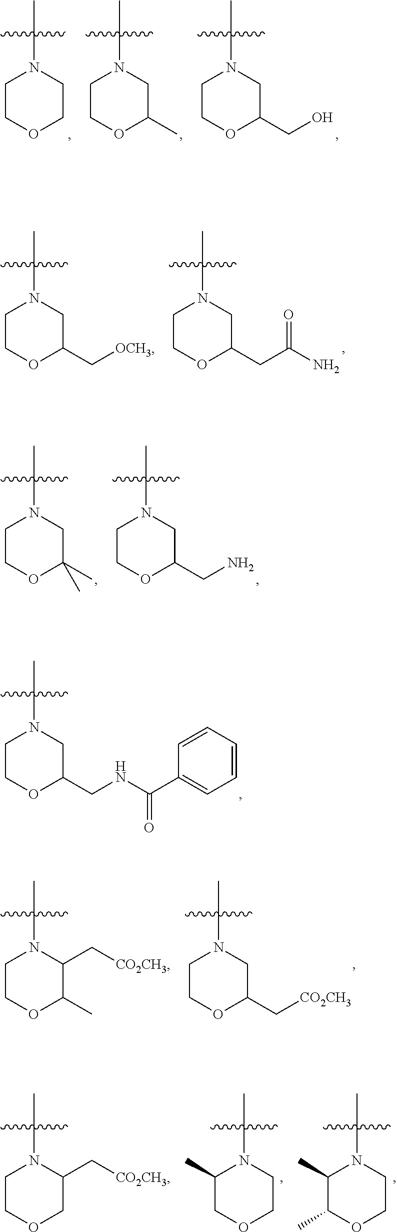

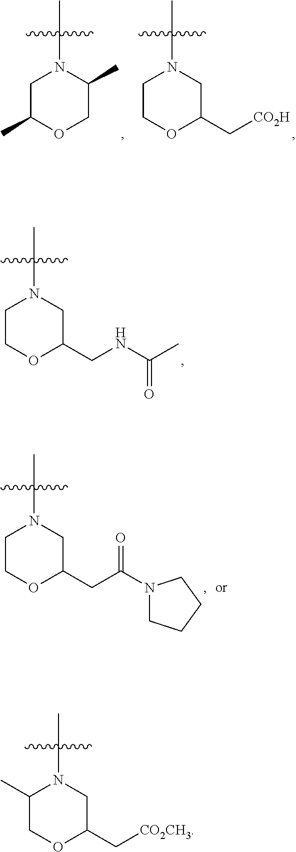

In certain embodiments, Ring A² is an optionally substituted ring 5-15 membered saturated or partially unsaturated bridged bicyclic heterocyclic ring having at least one nitrogen, at least one oxygen, and optionally 1-2 additional heteroatoms independently selected from nitrogen, oxygen, or sulfur. In certain embodiments, Ring A² is an optionally substituted ring 5-10 membered saturated or partially unsaturated bridged bicyclic heterocyclic ring having at least one nitrogen, at least one oxygen, and optionally 1-2 additional heteroatoms independently selected from nitrogen, oxygen, or sulfur. In certain embodiments, Ring A² is a bridged, bicyclic morpholino group. In certain embodiments, A² is an optionally substituted ring having the structure:

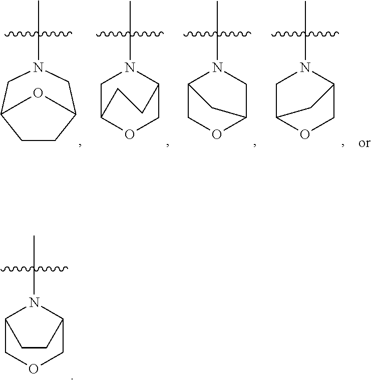

In certain embodiments, Ring A² is of the formula:

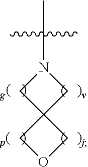

wherein:
v, j, p, and g are independently 1, 2, or 3.

In some embodiments, Ring A² is an optionally substituted ring having the structure:

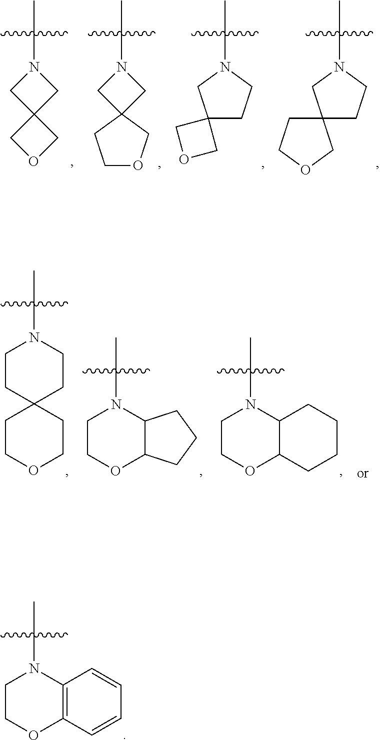

In certain embodiments, the T² group of either of formula II-e or II-f is a bivalent, straight, saturated C₁₋₆ hydrocarbon chain. In some embodiments, T² is a bivalent, straight, saturated C₁₋₃ hydrocarbon chain. In some embodiments, T² is —CH₂— or —CH₂CH₂—. In other embodiments, T² is —C(O)—. In certain embodiments, T² is —C≡C— or —CH₂C≡C—. In certain embodiments, T² is a covalent bond. In some embodiments, T² is a covalent bond, methylene, or a C₂₋₄ hydrocarbon chain wherein one methylene unit of T² is replaced by —C(O)NH—. In certain embodiments, T² is a C₃ hydrocarbon chain wherein one methylene unit of T² is replaced by —C(O)NH—.

In certain embodiments, the Ring C¹ group of either of formula II-e or II-f is an optionally substituted 6-membered saturated heterocyclic ring having one or two heteroatoms independently selected from nitrogen, oxygen, or sulfur. In some embodiments, Ring C¹ is a piperazinyl or piperidinyl ring. In some embodiments, Ring C¹ is an optionally substituted 6-membered partially unsaturated heterocyclic ring having one or two heteroatoms independently selected from nitrogen, oxygen, or sulfur. In certain embodiments, Ring C¹ is tetrahydropyridyl. In some embodiments, Ring C¹ is phenyl. In some embodiments, Ring C¹ is an optionally substituted 3-7 membered saturated or partially unsaturated carbocyclic ring. In certain embodiments, Ring C¹ is cyclohexyl. In certain embodiments, Ring C¹ is absent. In some embodiments, Ring C¹ is a 7-12 membered saturated or partially unsaturated bridged bicyclic ring having 0-4 heteroatoms independently selected from nitrogen, oxygen, or sulfur.

In certain embodiments, the T³ group of either of formula II-e or II-f is a bivalent, straight, saturated C₁₋₆ hydrocarbon chain. In some embodiments, T³ is a bivalent, straight, saturated C₁₋₃ hydrocarbon chain. In some embodiments, T³ is —CH₂— or —CH₂CH₂—. In certain embodiments, T³ is —C(O)—. In certain embodiments, T³ is a covalent bond.

In certain embodiments, the Ring D² group of either of formula II-e or II-f is an optionally substituted 6-membered saturated heterocyclic ring having one or two heteroatoms independently selected from nitrogen, oxygen, or sulfur. In some embodiments, Ring D² is a piperazinyl or piperidinyl ring. In some embodiments, Ring D² is an optionally substituted 6-membered partially unsaturated heterocyclic ring having one or two heteroatoms independently selected from nitrogen, oxygen, or sulfur. In certain embodiments, Ring D² is tetrahydropyridyl. In some embodiments, Ring D² is phenyl. In some embodiments, Ring D² is an optionally substituted 3-7 membered saturated or partially unsaturated carbocyclic ring. In certain embodiments, Ring D² is cyclohexyl. In certain embodiments, Ring D² is absent. In some embodiments, Ring D² is a 7-12 membered saturated or partially unsaturated bridged bicyclic ring having 0-4 heteroatoms independently selected from nitrogen, oxygen, or sulfur.

In certain embodiments, the present invention provides a compound of formula II-e-i or II-f-i:

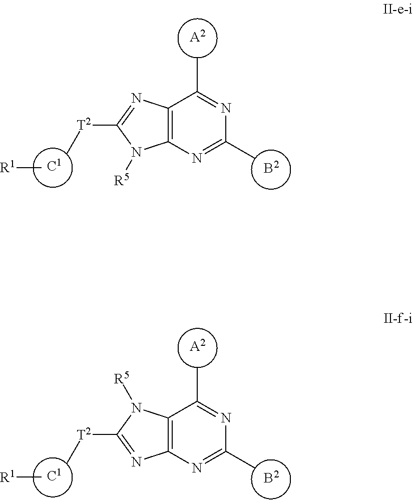

or a pharmaceutically acceptable salt thereof, wherein:
R¹, R⁵, R, Ring B², and T² are as defined above for formula II-e and II-f, and described in classes and subclasses herein;
Ring A² is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged bicyclic heterocyclic ring having at least one nitrogen, at least one oxygen, and optionally 1-2 additional heteroatoms independently selected from nitrogen, oxygen, or sulfur; and
Ring C¹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

It will be understood by one of ordinary skill in the art that when Ring C¹ of formula II-e-i or II-f-i is absent, R¹ is directly attached to T².

In certain embodiments, the present invention provides a compound of formula II-g or II-h:

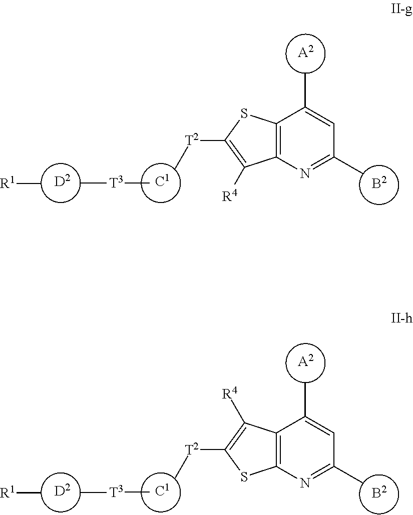

or a pharmaceutically acceptable salt thereof, wherein:
R¹ is a warhead group;
Ring A² is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R⁴ is —R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
   two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B² is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T² is a covalent bond or a bivalent straight or branched, saturated or unsaturated C₁₋₆ hydrocarbon chain wherein one or more methylene units of T² are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C¹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T³ is a covalent bond or a bivalent straight or branched, saturated or unsaturated C₁₋₆ hydrocarbon chain wherein one or more methylene units of T³ are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring D² is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

It will be understood by one of ordinary skill in the art that when Ring C¹ of formula II-g or II-h is absent, T³ is directly attached to T². It will be further understood that when Ring D² is absent, R¹ is directly attached to T³.

In certain embodiments, the Ring B² group of either of formula II-g or II-h is an optionally substituted 8-10 membered bicyclic heteroaryl ring having 1-4 heteroatoms independently selected from nitrogen, oxygen, or sulfur. In some embodiments, Ring B² is an optionally substituted 8-10 membered bicyclic heteroaryl ring having 2 nitrogen atoms. In some embodiments, Ring B² is 1H-indazolyl, benzimidazolyl, or indolyl. In certain embodiments, Ring B² is 1H-indazolyl. In certain embodiments, the Ring B² group is substituted or unsubstituted phenyl. In certain embodiments, Ring B² is substituted phenyl. In certain embodiments, Ring B² is phenol. In some embodiments, Ring B² is a 5-6 membered heteroaryl ring having 1-4 heteroatoms independently selected from nitrogen, oxygen, or sulfur. In some embodiments, Ring B² is an optionally substituted 5-6 membered heteroaryl ring having 1-2 nitrogen atoms. In certain embodiments, Ring B² is pyridyl. In certain embodiments, Ring B² is optionally substituted pyrimidinyl. In certain embodiments, Ring B² is

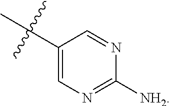

In certain embodiments, the Ring A² group of either of formula II-g or II-h is an optionally substituted 5-6 membered saturated or partially unsaturated heterocyclic ring having one or two heteroatoms independently selected from nitrogen, oxygen, or sulfur. In some embodiments, Ring A² is an optionally substituted 6-membered saturated or partially unsaturated heterocyclic ring having one or two heteroatoms independently selected from nitrogen, oxygen, or sulfur. In some embodiments, Ring A² is optionally substituted morpholinyl. In certain embodiments, Ring A² is unsubstituted morpholinyl. In some embodiments, Ring A² is optionally substituted tetrahydropyranyl. In certain embodiments, A² is:

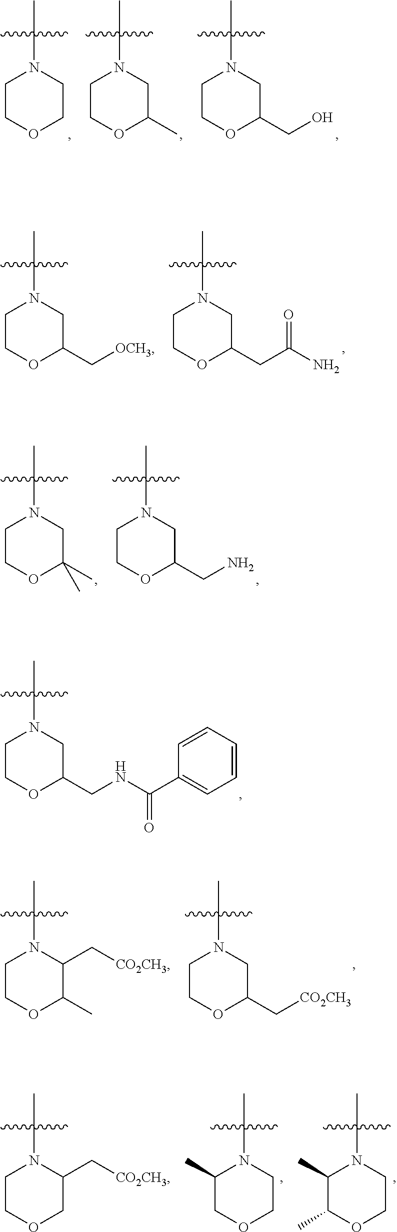

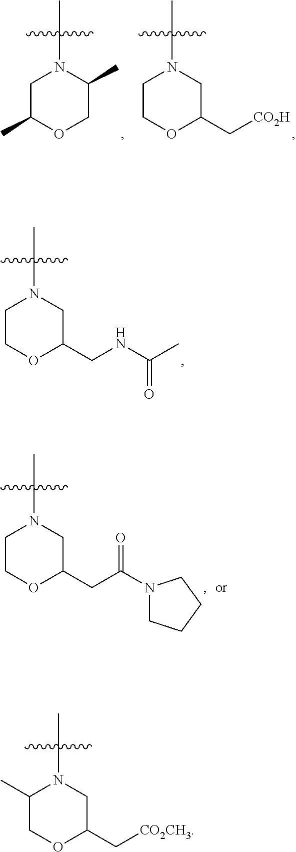

In certain embodiments, Ring A² is an optionally substituted ring 5-15 membered saturated or partially unsaturated bridged bicyclic heterocyclic ring having at least one nitrogen, at least one oxygen, and optionally 1-2 additional heteroatoms independently selected from nitrogen, oxygen, or sulfur. In certain embodiments, Ring A² is an optionally substituted ring 5-10 membered saturated or partially unsaturated bridged bicyclic heterocyclic ring having at least one nitrogen, at least one oxygen, and optionally 1-2 additional heteroatoms independently selected from nitrogen, oxygen, or sulfur. In certain embodiments, Ring A² is a bridged, bicyclic morpholino group. In certain embodiments, A² is an optionally substituted ring having the structure:

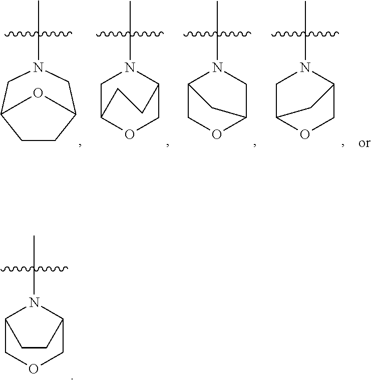

In certain embodiments, Ring A² is of the formula:

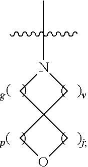

wherein:
v, j, p, and g are independently 1, 2, or 3.

In some embodiments, Ring A² is an optionally substituted bicyclic (fused or spiro-fused) ring selected from:

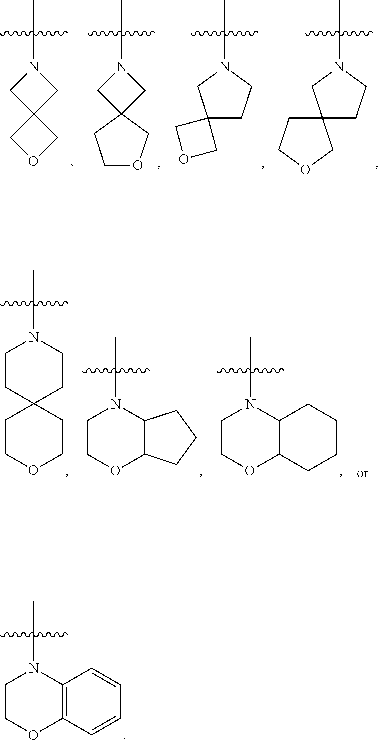

In certain embodiments, the T² group of either of formula II-g or II-h is a bivalent, straight, saturated C₁₋₆ hydrocarbon chain. In some embodiments, T² is a bivalent, straight, saturated C₁₋₃ hydrocarbon chain. In some embodiments, T² is —CH₂— or —CH₂CH₂—. In other embodiments, T² is —C(O)—. In certain embodiments, T² is —C≡C— or —CH₂C≡C—. In certain embodiments, T² is a covalent bond. In some embodiments, T² is a covalent bond, methylene, or a C₂₋₄ hydrocarbon chain wherein one methylene unit of T² is replaced by —C(O)NH—. In certain embodiments, T² is a C₃ hydrocarbon chain wherein one methylene unit of T² is replaced by —C(O)NH—.

In certain embodiments, the Ring C¹ group of either of formula II-g or II-h is an optionally substituted 6-membered saturated heterocyclic ring having one or two heteroatoms independently selected from nitrogen, oxygen, or sulfur. In some embodiments, Ring C¹ is a piperazinyl or piperidinyl ring. In some embodiments, Ring C¹ is an optionally substituted 6-membered partially unsaturated heterocyclic ring having one or two heteroatoms independently selected from nitrogen, oxygen, or sulfur. In certain embodiments, Ring C¹ is tetrahydropyridyl. In some embodiments, Ring C¹ is phenyl. In some embodiments, Ring C¹ is an optionally substituted 3-7 membered saturated or partially unsaturated carbocyclic ring. In certain embodiments, Ring C¹ is cyclohexyl. In certain embodiments, Ring C¹ is absent. In some embodiments, Ring C¹ is a 7-12 membered saturated or partially unsaturated bridged bicyclic ring having 0-4 heteroatoms independently selected from nitrogen, oxygen, or sulfur.

In certain embodiments, the T³ group of either of formula II-g or II-h is a bivalent, straight, saturated C₁₋₆ hydrocarbon chain. In some embodiments, T³ is a bivalent, straight, saturated C₁₋₃ hydrocarbon chain. In some embodiments, T³ is —CH₂— or —CH₂CH₂—. In certain embodiments, T³ is —C(O)—. In certain embodiments, T³ is a covalent bond.

In certain embodiments, the Ring D² group of either of formula II-g or II-h is an optionally substituted 6-membered saturated heterocyclic ring having one or two heteroatoms independently selected from nitrogen, oxygen, or sulfur. In some embodiments, Ring D² is a piperazinyl or piperidinyl ring. In some embodiments, Ring D² is an optionally substituted 6-membered partially unsaturated heterocyclic ring having one or two heteroatoms independently selected from nitrogen, oxygen, or sulfur. In certain embodiments, Ring D² is tetrahydropyridyl. In some embodiments, Ring D² is phenyl. In some embodiments, Ring D² is an optionally substituted 3-7 membered saturated or partially unsaturated carbocyclic ring. In certain embodiments, Ring D² is cyclohexyl. In certain embodiments, Ring D² is absent. In some embodiments, Ring D² is a 7-12 membered saturated or partially unsaturated bridged bicyclic ring having 0-4 heteroatoms independently selected from nitrogen, oxygen, or sulfur.

In some embodiments, a provided compound of formula II-g or II-h has one or more, more than one, or all of the features selected from:

a1) R¹ is selected from those embodiments described herein;
b1) Ring A² is selected from those embodiments described for formulae II-g and II-h, above;
c1) Ring B² is selected from those embodiments described for formulae II-g and II-h, above;
d1) T² is selected from those embodiments described for formulae II-g and II-h, above;
e1) Ring C¹ is selected from those embodiments described for formulae II-g and II-h, above;
f1) T³ is selected from those embodiments described for formulae II-g and II-h, above; and
g1) Ring D² is selected from those embodiments described for formulae II-g and II-h, above.

In some embodiments,

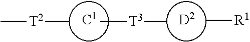

of formula II-g or II-h is

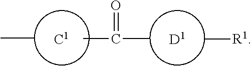

In some embodiments,

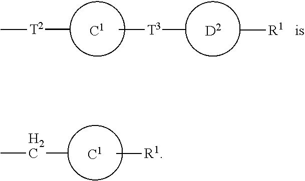

In some embodiments,

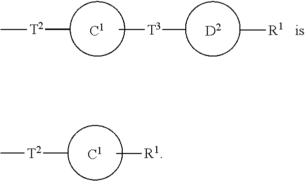

In some embodiments, a provided compound of formula II-g or II-h has one or more, more than one, or all of the features selected from:

a2) Ring A² is optionally substituted morpholinyl;
b2) Ring B² is an optionally substituted 8-10 membered bicyclic heteroaryl ring having 1-2 nitrogen atoms, optionally substituted phenyl, or an optionally substituted 5-6 membered heteroaryl ring having 1-2 nitrogen atoms;
c2)

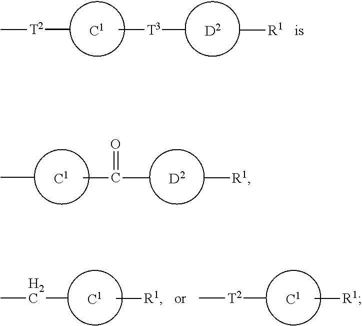

and
d2)

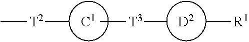

comprises a spacer group as defined herein having about 9 to about 11 atoms. In some embodiments, a provided compound of formula II-g or II-h has one or more, more than one, or all of the features selected from: a2), b2), c2), and d2) described above, and e2) R¹ is selected from those embodiments described herein.

In some embodiments, a provided compound of formula II-g or II-h has one or more, more than one, or all of the features selected from:

a3) Ring A² is optionally substituted morpholinyl;
b3) Ring B² is an optionally substituted group selected from indazolyl, aminopyrimidinyl, or phenol;
c3)

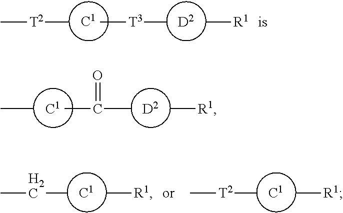

and
d3)

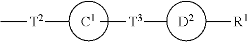

comprises a spacer group having about 9 to about 11 atoms. In some embodiments, a provided compound of formula II-g or II-h has one or more, more than one, or all of the features selected from: a3), b3), c3), and d3) described above, and e3) R¹ is selected from those embodiments described herein.

In some embodiments, a provided compound of formula II-g or II-h has one or more, more than one, or all of the features selected from:

a4) Ring A² is optionally substituted morpholinyl;
b4) Ring B² is an optionally substituted 8-10 membered bicyclic heteroaryl ring having 1-2 nitrogen atoms, optionally substituted phenyl, or an optionally substituted 5-6 membered heteroaryl ring having 1-2 nitrogen atoms;
c4) T² is a covalent bond, methylene, or a C₃₋₅ hydrocarbon chain wherein 2 methylene units of T² are replaced by —C(O)NH—;
d4) Ring C¹ is phenyl, or an optionally substituted 6-membered saturated, partially unsaturated, or aromatic heterocyclic ring having 1-2 nitrogens;
e4) T³ is a covalent bond, —C(O)—; and
f4) Ring D² is absent or phenyl.

In some embodiments, a provided compound of formula II-g or II-h has one or more, more than one, or all of the features selected from: a4), b4), c4), d4), e4), and f4) described above, and g4) R¹ is selected from those embodiments described herein.

In some embodiments, a provided compound of formula II-g or II-h has one or more, more than one, or all of the features selected from:

a5) Ring A² is optionally substituted morpholinyl;
b5) Ring B² is an optionally substituted group selected from indazolyl, phenol, or aminopyrimidine;
c5) T² is a covalent bond, methylene, or a C₄ hydrocarbon chain wherein 2 methylene units of T² are replaced by —C(O)NH—;
d5) Ring C¹ is phenyl, piperazinyl, piperidinyl, or tetrahydropyridyl;
e5) T³ is a covalent bond or —C(O)—; and
f5) Ring D² is absent or phenyl.
In some embodiments, a provided compound of formula II-g or II-h has one or more, more than one, or all of the features selected from: a5), b5), c5), d5), e5), and f5) described above, and g5) R¹ is selected from those embodiments described herein.

In some embodiments, the length or number of atoms from the II-a, II-b, II-e, II-f, II-g, or II-h scaffold to the reactive moiety of the warhead group contributes to selective modification of Cys-862 of PI3Kα. It will be appreciated that such length, i.e. number of atoms, places the reactive moiety of the warhead group within proximity of Cys-862 of PI3Kα to achieve covalent modification. As used herein, the term “scaffold” refers to a) a radical resulting from the removal of a hydrogen of a ligand capable of binding to, or in proximity to, the ligand-binding site; or b) a portion of a pharmacophore of a ligand resulting from truncation of the pharmacophore, such that the scaffold is capable of binding to, or in proximity to, the ligand-binding site. II-a, II-b, II-e, II-f, II-g, or II-h scaffolds are shown below.

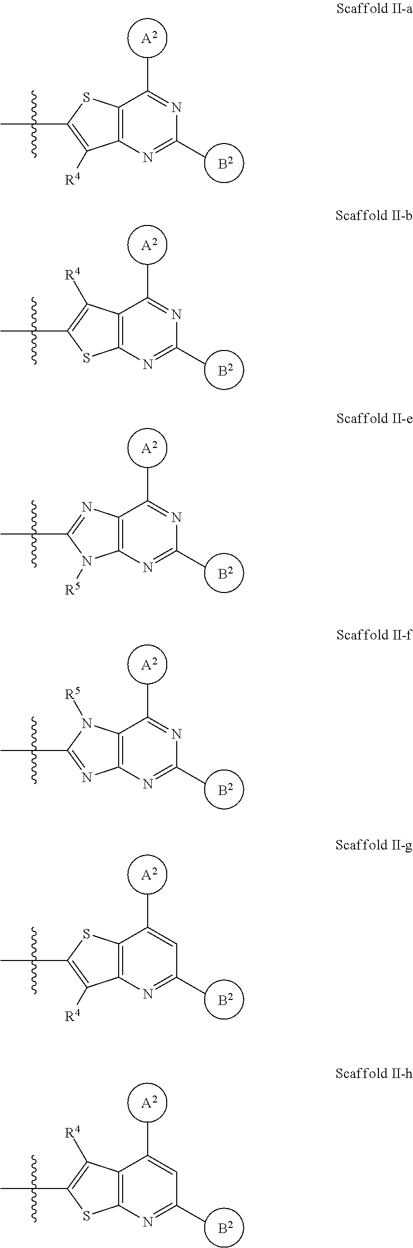

It will be appreciated that the

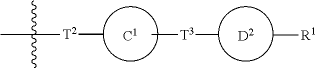

group of formulae II-a, II-b, II-e, II-f, II-g, and II-h acts as a spacer group between the scaffold and the reactive moiety of the R¹ warhead. The term “spacer group” refers to a group that separates and orients other parts of the molecule attached thereto, such that the compound favorably interacts with functional groups in the active site of an enzyme. As used herein, the spacer group separates and orients the scaffold and the reactive moiety of R¹ within the active site such that they may form favorable interactions with functional groups which exist within the active site of PI3Kα and such that R¹ may react with Cys-862. It will be appreciated that a spacer group begins with the first atom attached to the scaffold and ends with the reactive center of the warhead (e.g., reactive carbon center as identified in structure below as atom 11).

In some embodiments, a spacer group is from about 7 atoms to about 13 atoms in length. In some embodiments, a spacer group is from about 8 atoms to about 12 atoms in length. In some embodiments, a spacer group is from about 9 atoms to about 11 atoms in length. For purposes of counting spacer group length when a ring is present in the spacer group, the ring is counted as three atoms from one end to the other. For example, the spacer group portion of the

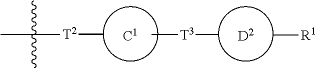

group shown below will be understood to be 11 atoms long. The wavy line indicates the point of attachment to the scaffold.

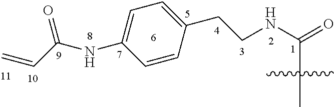

In some embodiments, a spacer group is from about 6 Å to about 12 Å in length. In some embodiments, a spacer group is from about 5 Å to about 11 Å in length. In some embodiments, a spacer group is from about 6 Å to about 9 Å in length.

For avoidance of doubt and for illustrative purposes, exemplary compounds are shown below with the length of their spacers.

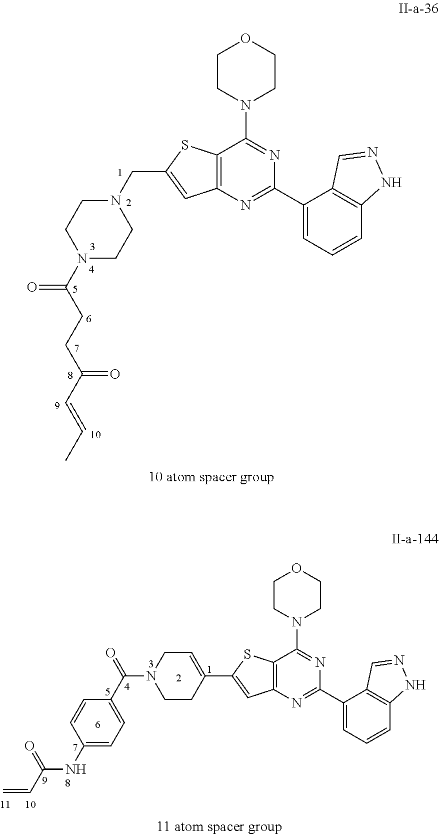

In some embodiments, the present invention provides a compound of formula III:

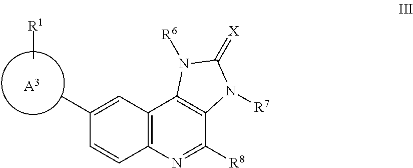

or a pharmaceutically acceptable salt thereof, wherein:
R¹ is a warhead group;
X is O or S;
R⁶ is an optionally substituted group selected from phenyl, napthyl, a 6-membered heteroaryl ring having 1-2 nitrogens, or an 8-10 membered bicyclic heteroaryl ring having 1-3 heteroatoms independently selected from nitrogen, oxygen, or sulfur;
R⁷ is an optionally substituted C₁₋₆ aliphatic group;
R⁸ is hydrogen or —NHR′;
R′ is independently hydrogen or an optionally substituted C₁₋₆ aliphatic group; and
Ring A³ is an optionally substituted group selected from phenyl, naphthyl, a 6-membered heteroaryl ring having 1-2 nitrogens, or an 8-10 membered bicyclic heteroaryl ring having 1-3 nitrogens.

In certain embodiments, the present invention provides a compound of formula III selected from formulae III-a, III-b, and III-c:

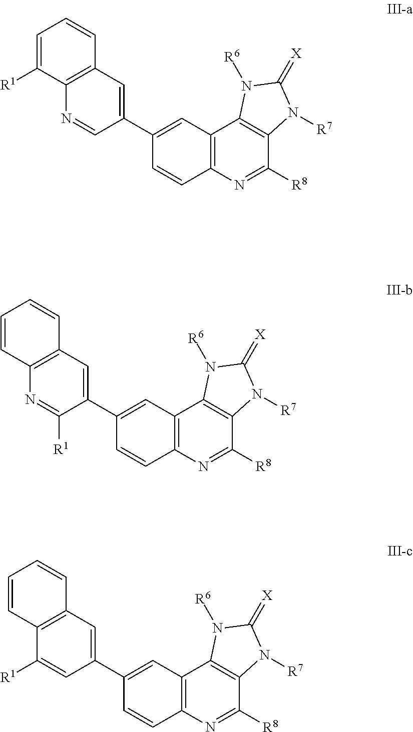

wherein each of R¹, R⁶, R⁷, R⁸, and X is as defined above for formula III and as described herein.

In certain embodiments, the X group of formula III is O. In other embodiments, the X group of formula III is S.

In certain embodiments, the R⁶ group of formula III is an optionally substituted phenyl. In some embodiments, R⁶ is phenyl substituted with R^{∘}. In other embodiments, R⁶ is phenyl substituted with cyano-substituted C₁₋₆ alkyl. In some embodiments, R⁶ is phenyl substituted with —C(CH₃)₂CN.

In some embodiments, the R⁷ group of formula III is an optionally substituted C₁₋₆ alkyl group. In other embodiments, R⁷ is a C₁₋₃ alkyl group. In certain embodiments, R⁷ is methyl, ethyl, propyl, or cyclopropyl.

In certain embodiments, the R⁸ group of formula III is hydrogen.

In certain embodiments, the Ring A³ group of formula III is phenyl, pyridyl, pyrimidinyl, pyrazinyl, naphthyl, or quinolinyl.

In some embodiments, the present invention provides a compound of formula IV:

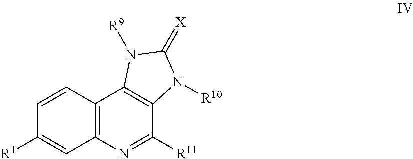

or a pharmaceutically acceptable salt thereof, wherein:
R¹ is a warhead group;
X is O or S;
R⁹ is an optionally substituted group selected from phenyl, napthyl, a 6-membered heteroaryl ring having 1-2 nitrogens, or an 8-10 membered bicyclic heteroaryl ring having 1-3 heteroatoms independently selected from nitrogen, oxygen, or sulfur;
R¹⁰ is an optionally substituted C₁₋₆ aliphatic group;
R¹¹ is hydrogen or —NHR′; and
R′ is independently hydrogen or an optionally substituted C₁₋₆ aliphatic group.

In certain embodiments, the X group of formula IV is O. In other embodiments, the X group of formula IV is S.

In certain embodiments, the R⁹ group of formula IV is an optionally substituted phenyl. In some embodiments, R⁹ is phenyl substituted with R^{∘}. In other embodiments, R⁹ is phenyl substituted with cyano-substituted C₁₋₆ alkyl. In some embodiments, R⁹ is phenyl substituted with —C(CH₃)₂CN.

In some embodiments, the R¹⁰ group of formula IV is an optionally substituted C₁₋₆ alkyl group. In other embodiments, R¹⁰ is a C₁₋₃ alkyl group. In certain embodiments, R¹⁰ is methyl, ethyl, propyl, or cyclopropyl.

In certain embodiments, the R⁴ group of formula IV is hydrogen.

In some embodiments, the present invention provides a compound of formula V-a or V-b:

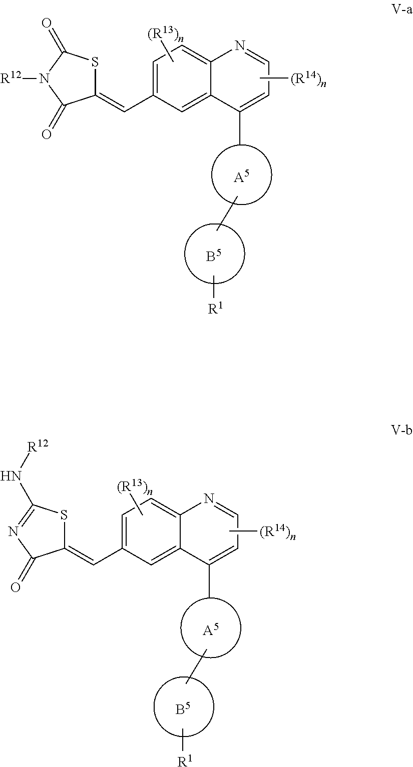

or a pharmaceutically acceptable salt thereof, wherein:
R¹ is a warhead group;
R¹² is an hydrogen or an optionally substituted group selected from C₁₋₆ aliphatic, —(CH₂)ₘ-(3-7 membered saturated or partially unsaturated carbocyclic ring), —(CH₂)ₘ-(7-10 membered saturated or partially unsaturated bicyclic carbocyclic ring), —(CH₂)ₘ-(4-7 membered saturated or partially unsaturated heterocyclic ring having 1-2 heteroatoms independently selected from nitrogen, oxygen, or sulfur), —(CH₂)ₘ-(7-10 membered saturated or partially unsaturated bicyclic heterocyclic ring having 1-3 heteroatoms independently selected from nitrogen, oxygen, or sulfur), —(CH₂)ₘ-phenyl, —(CH₂)ₘ-(8-10 membered bicyclic aryl ring), —(CH₂)ₘ-(5-6 membered heteroaryl ring having 1-3 heteroatoms independently selected from nitrogen, oxygen, or sulfur), or —(CH₂)ₘ-(8-10 membered bicyclic heteroaryl ring having 1-4 heteroatoms independently selected from nitrogen, oxygen, or sulfur);
each R¹³ and R¹⁴ is independently —R″, halogen, —NO₂, —CN, —OR″, —SR″, —N(R″)₂, —C(O)R″, —CO₂R″, —C(O)C(O)R″, —C(O)CH₂C(O)R″, —S(O)R″, —S(O)₂R″, —C(O)N(R″)₂, —SO₂N(R″)₂, —OC(O)R″, —N(R″)C(O)R″, —N(R″)N(R″)₂, —N(R″)C(═NR″)N(R″)₂, —C(═NR″)N(R″)₂, —C═NOR″, —N(R″)C(O)N(R″)₂, —N(R″)SO₂N(R″)₂, —N(R″)SO₂R″, or —OC(O)N(R″)₂;
each R″ is independently hydrogen or an optionally substituted group selected from C₁₋₆ aliphatic, a 3-7 membered saturated or partially unsaturated carbocyclic ring, a 7-10 membered saturated or partially unsaturated bicyclic carbocyclic ring, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; or
   two R″ groups on the same nitrogen are taken together with the nitrogen to which they are attached to form an optionally substituted 5-8 membered saturated, partially unsaturated, or aromatic ring having 1-4 heteroatoms independently selected from nitrogen, oxygen, or sulfur;
m is an integer from 0 to 6, inclusive;
each n is independently 0, 1, or 2;
Ring A⁵ is an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; and
Ring B⁵ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

It will be understood by one of ordinary skill in the art that when Ring B⁵ is absent, R¹ is directly attached to Ring A⁵.

In certain embodiments, the R¹² group of formulae V-a and V-b is hydrogen. In some embodiments, R¹² is C₁₋₆ aliphatic. In certain embodiments, R¹² is C₁₋₆ alkyl. In some embodiments, R¹² is methyl. In certain embodiments, R¹² is optionally substituted phenyl. In some embodiments, R¹² is phenyl substituted with one or more halogens. In certain embodiments, R¹² is dichlorophenyl. In some embodiments, R¹² is aralkyl or heteroaralkyl. In certain embodiments, R¹² is optionally substituted benzyl. In some embodiments, R¹² is an optionally substituted group selected from a 3-7 membered saturated or partially unsaturated carbocyclic ring, a 7-10 membered saturated or partially unsaturated bicyclic carbocyclic ring, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, a 8-10 membered bicyclic aryl ring, a 5-6 membered heteroaryl ring having 1-3 heteroatoms independently selected from nitrogen, oxygen, or sulfur, or a 8-10 membered bicyclic heteroaryl ring having 1-4 heteroatoms independently selected from nitrogen, oxygen, or sulfur. In some embodiments, the R¹² group of formula V-a is hydrogen. In certain embodiments, the R¹² group of formula V-b is substituted phenyl.

In some embodiments, Ring A⁵ of formulae V-a and V-b is an optionally substituted 6-membered heterocyclic ring having 1-2 nitrogens. In certain embodiments, Ring A⁵ is a piperidine ring. In certain embodiments, Ring A⁵ is a piperazine ring. In some embodiments, Ring A⁵ is an optionally substituted 6-membered heteroaryl ring having 1-2 nitrogens. In certain embodiments, Ring A⁵ is a pyridine ring. In certain embodiments, Ring A⁵ is a pyrimidine ring. In certain embodiments, Ring A⁵ is a pyrazine ring. In certain embodiments, Ring A⁵ is a pyridazine ring.

In some embodiments, Ring A⁵ is optionally substituted phenyl. In some embodiments, Ring A⁵ is an optionally substituted 8-10 membered bicyclic heteroaryl ring having 1-4 heteroatoms independently selected from nitrogen, oxygen, or sulfur. In certain embodiments, Ring A⁵ is a tetrahydroisoquinoline ring.

In some embodiments, Ring B⁵ of formulae V-a and V-b is an optionally substituted 6-membered heterocyclic ring having 1-2 nitrogens. In certain embodiments, Ring B⁵ is a piperidine ring. In certain embodiments, Ring B⁵ is a piperazine ring. In some embodiments, Ring B⁵ is an optionally substituted 6-membered heteroaryl ring having 1-2 nitrogens. In certain embodiments, Ring B⁵ is a pyridine ring. In certain embodiments, Ring B⁵ is a pyrimidine ring. In certain embodiments, Ring B⁵ is a pyrazine ring. In certain embodiments, Ring B⁵ is a pyridazine ring. In some embodiments, Ring B⁵ is phenyl. In some embodiments, Ring B⁵ is a 3-7 membered saturated or partially unsaturated carbocyclic ring. In certain embodiments, Ring B⁵ is cyclohexyl.

In certain embodiments, n of formulae V-a and V-b is 0. In some embodiments, n is 1. In other embodiments, n is 2.

In some embodiments, the present invention provides a compound of formula V-a-i or V-b-i:

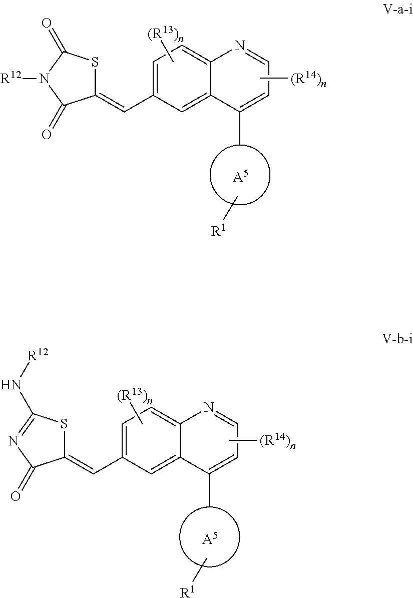

or a pharmaceutically acceptable salt thereof, wherein:
R¹, R¹², R¹³, R¹⁴, R″, m, and n are as defined above for formulae V-a and V-b above and described in classes and subclasses herein; and
   Ring A⁵ is an optionally substituted 6-membered heterocyclic or heteroaryl ring having 1-2 nitrogens.

In some embodiments, the present invention provides a compound of formula VI-a or VI-b:

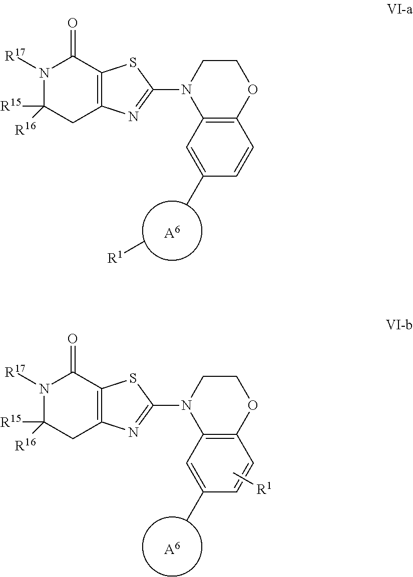

or a pharmaceutically acceptable salt thereof, wherein:
R¹ is a warhead group;
R¹⁵ is hydrogen or C₁₋₆ alkyl;
R¹⁶ is hydrogen or an optionally substituted group selected from C₁₋₆ alkyl, C₁₋₆ alkoxy, or (C₁₋₆ alkylene)-R¹⁸; or
R¹⁵ and R¹⁶ are taken together with the intervening carbon to form an optionally substituted ring selected from a 3-7 membered carbocyclic ring or a 4-7 membered heterocyclic ring having 1-2 heteroatoms independently selected from nitrogen, oxygen, or sulfur;
R¹⁷ is hydrogen or C₁₋₆ alkyl;
R¹⁸ is a 3-7 membered saturated or partially unsaturated carbocyclic ring, a 7-10 membered saturated or partially unsaturated bicyclic carbocyclic ring, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, a 8-10 membered bicyclic aryl ring, a 5-6 membered heteroaryl ring having 1-3 heteroatoms independently selected from nitrogen, oxygen, or sulfur, or a 8-10 membered bicyclic heteroaryl ring having 1-4 heteroatoms independently selected from nitrogen, oxygen, or sulfur; and
Ring A⁶ is absent or an optionally substituted group selected from a 4-7 membered heterocyclic ring having 1-2 heteroatoms independently selected from nitrogen, oxygen, or sulfur, or a 5-6 membered heteroaryl ring having 1-3 heteroatoms independently selected from nitrogen, oxygen, or sulfur.

In certain embodiments, R¹⁵ of formulae VI-a and VI-b is hydrogen. In some embodiments, R¹⁵ is C₁₋₆ alkyl. In some embodiments, R¹⁵ is methyl.

In some embodiments, R¹⁶ of formulae VI-a and VI-b is hydrogen. In some embodiments, R¹⁶ is C₁₋₆ alkyl. In certain embodiments, R¹⁶ is methyl.

In some embodiments, R¹⁷ of formulae VI-a and VI-b is hydrogen. In some embodiments, R¹⁷ is C₁₋₆ alkyl. In certain embodiments, R¹⁷ is methyl.

In some embodiments, Ring A⁶ of formulae VI-a and VI-b is 4-7 membered heterocyclic ring having 1-2 heteroatoms independently selected from nitrogen, oxygen, or sulfur. In some embodiments, Ring A⁶ is a 5-6 membered heteroaryl ring having 1-3 heteroatoms independently selected from nitrogen, oxygen, or sulfur. In certain embodiments, Ring A⁶ is a 5-membered heteroaryl ring having two nitrogens. In certain embodiments, Ring A⁶ is pyrazolyl.

In certain embodiments, Ring A⁶ of formula VI-a or VI-b is absent. It is to be understood that when Ring A⁶ is absent in formula VI-a, R¹ is covalent attached to the benzomorpholine ring at the position meta to the morpholine nitrogen. It is to be understood that when Ring A⁶ is absent in formula VI-b, R¹ can be attached to any position on the benzomorpholine ring, and valency of the benzomorpholine ring is satisfied with a hydrogen or optional substituent.

In certain embodiments, the present invention provides a compound of formula VII:

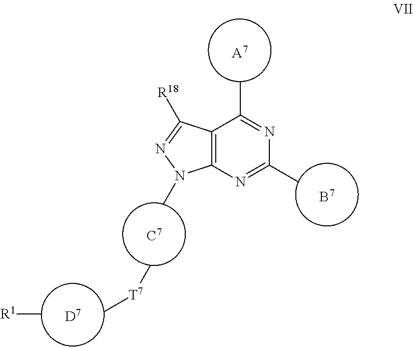

or a pharmaceutically acceptable salt thereof, wherein:
R¹ is a warhead group;
Ring A⁷ is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R¹⁸ is R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
   two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B⁷ is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T⁷ is a covalent bond or a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C⁷ is an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; and
Ring D⁷ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

One of ordinary skill in the art will appreciate that when Ring D⁷ of formula VII is absent, R¹ is directly attached to T⁷.

In certain embodiments, the Ring B⁷ group of formula VII is an optionally substituted 8-10 membered bicyclic heteroaryl ring having 1-4 heteroatoms independently selected from nitrogen, oxygen, or sulfur. In some embodiments, Ring B⁷ is an optionally substituted 8-10 membered bicyclic heteroaryl ring having 2 nitrogen atoms. In some embodiments, Ring B⁷ is 1H-indazolyl, benzimidazolyl, or indolyl. In certain embodiments, Ring B⁷ is 1H-indazolyl. In certain embodiments, the Ring B⁷ group is substituted or unsubstituted phenyl. In certain embodiments, Ring B⁷ is substituted phenyl. In certain embodiments, Ring B⁷ is phenol. In certain embodiments, Ring B⁷ is phenyl substituted with —NHCOCH₃, —NHCOCH₂CH₃, —NHCO₂CH₂CH₂OH, —NHCONHCH₃, or —NHCONH(pyridyl). In certain embodiments, Ring B⁷ is phenyl substituted with —NHCO₂CH₃, —NHCONHCH₂CH₃, —NHCONHCH₂CH₂F, —NHCONHCH(CH₃)₂, —NHCONH(3-pyridyl), or —NHCONH(4-pyridyl). In certain embodiments, Ring B⁷ is

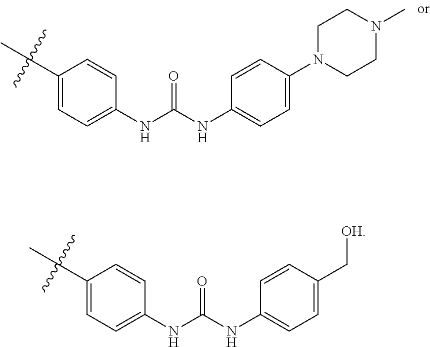

In some embodiments, Ring B⁷ is a 5-6 membered heteroaryl ring having 1-4 heteroatoms independently selected from nitrogen, oxygen, or sulfur. In some embodiments, Ring B⁷ is an optionally substituted 5-6 membered heteroaryl ring having 1-2 nitrogen atoms. In certain embodiments, Ring B⁷ is pyridyl. In certain embodiments, Ring B⁷ is optionally substituted pyrimidinyl. In certain embodiments, Ring B⁷ is

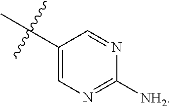

In certain embodiments, the Ring A⁷ group of formula VII is an optionally substituted 5-6 membered saturated or partially unsaturated heterocyclic ring having one or two heteroatoms independently selected from nitrogen, oxygen, or sulfur. In some embodiments, Ring A⁷ is an optionally substituted 6-membered saturated or partially unsaturated heterocyclic ring having one or two heteroatoms independently selected from nitrogen, oxygen, or sulfur. In some embodiments, Ring A⁷ is optionally substituted morpholinyl. In certain embodiments, Ring A⁷ is unsubstituted morpholinyl. In some embodiments, Ring A⁷ is optionally substituted tetrahydropyranyl. In certain embodiments, A⁷ is:

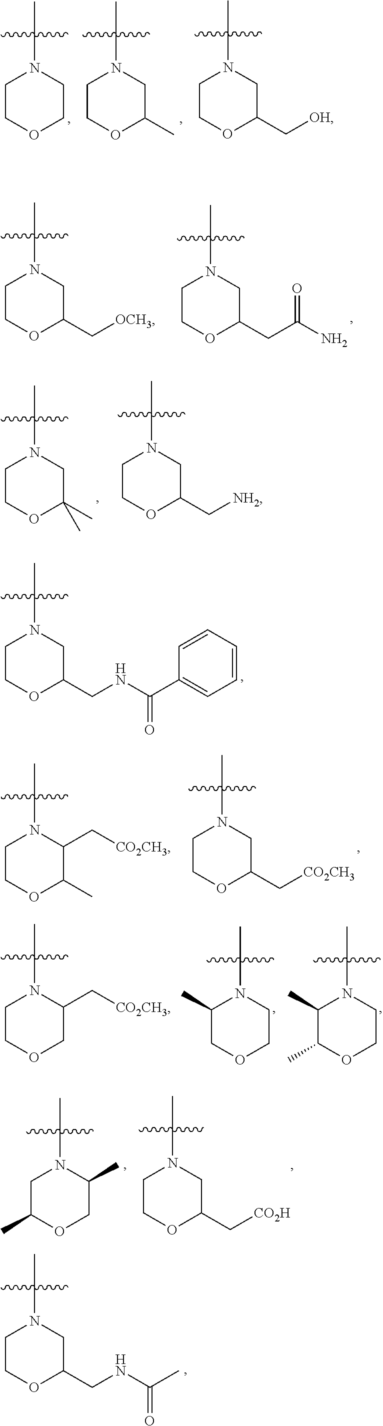

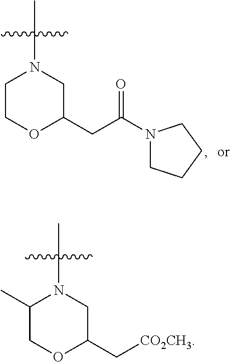

In certain embodiments, Ring A⁷ is an optionally substituted ring 5-15 membered saturated or partially unsaturated bridged bicyclic heterocyclic ring having at least one nitrogen, at least one oxygen, and optionally 1-2 additional heteroatoms independently selected from nitrogen, oxygen, or sulfur. In certain embodiments, Ring A⁷ is an optionally substituted ring 5-10 membered saturated or partially unsaturated bridged bicyclic heterocyclic ring having at least one nitrogen, at least one oxygen, and optionally 1-2 additional heteroatoms independently selected from nitrogen, oxygen, or sulfur. In certain embodiments, Ring A⁷ is a bridged, bicyclic morpholino group. In certain embodiments, A⁷ is an optionally substituted ring having the structure:

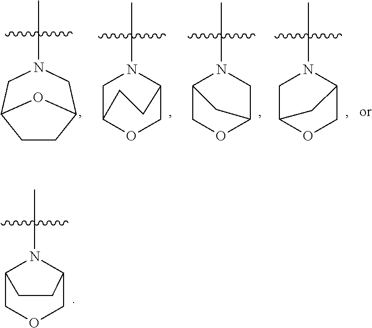

In certain embodiments, Ring A⁷ is of the formula:

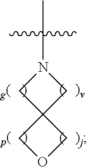

wherein:
v, j, p, and g are independently 1, 2, or 3.

In some embodiments, Ring A⁷ is an optionally substituted bicyclic (fused or spiro-fused) ring selected from:

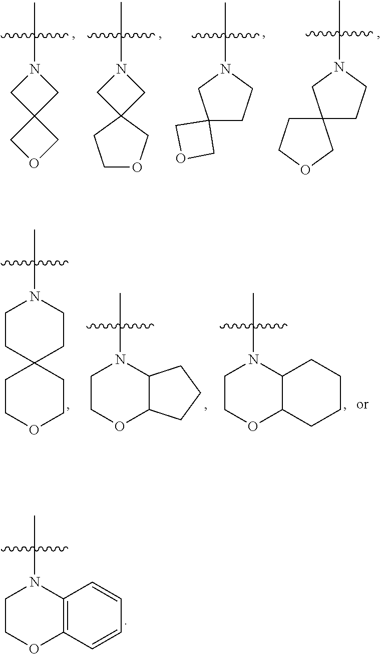

In certain embodiments, the T⁷ group of formula VII is a bivalent, straight, saturated C₁₋₆ hydrocarbon chain. In some embodiments, T⁷ is a bivalent, straight, saturated C₁₋₃ hydrocarbon chain. In some embodiments, T⁷ is —CH₂—. In certain embodiments, T⁷ is a covalent bond. In certain embodiments, T⁷ is —C(O)— or —CH₂C(O)—.

In certain embodiments, the Ring C⁷ group of formula VII is an optionally substituted 6-membered saturated heterocyclic ring having one or two heteroatoms independently selected from nitrogen, oxygen, or sulfur. In some embodiments, Ring C⁷ is a piperazinyl or piperidinyl ring. In certain embodiments, Ring C⁷ is piperidinyl. In certain embodiments, Ring C⁷ is substituted with one or more oxo groups. In certain embodiments, Ring C⁷ is thiomorpholine optionally substituted with one or more oxo groups. In some embodiments, Ring C⁷ is an optionally substituted 6-membered partially unsaturated heterocyclic ring having one or two heteroatoms independently selected from nitrogen, oxygen, or sulfur. In certain embodiments, Ring C⁷ is tetrahydropyridyl. In some embodiments, Ring C⁷ is phenyl. In some embodiments, C⁷ is an optionally substituted 5-6 membered heteroaryl ring having 1-3 heteroatoms independently selected from nitrogen, oxygen, or sulfur. In certain embodiments, Ring C⁷ is pyridyl. In some embodiments, Ring C⁷ is an optionally substituted 3-7 membered saturated or partially unsaturated carbocyclic ring. In certain embodiments, Ring C⁷ is cyclohexyl. In some embodiments, Ring C⁷ is a 7-12 membered saturated or partially unsaturated bridged bicyclic ring having 0-4 heteroatoms independently selected from nitrogen, oxygen, or sulfur.

In certain embodiments, the Ring D⁷ group of formula VII is an optionally substituted 6-membered saturated heterocyclic ring having one or two heteroatoms independently selected from nitrogen, oxygen, or sulfur. In some embodiments, Ring D⁷ is a piperazinyl or piperidinyl ring. In certain embodiments, Ring D⁷ is piperidinyl. In certain embodiments, Ring D⁷ is substituted with one or more oxo groups. In certain embodiments, Ring D⁷ is thiomorpholine optionally substituted with one or more oxo groups. In certain embodiments, Ring D⁷ is

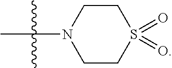

In some embodiments, Ring D⁷ is an optionally substituted 6-membered partially unsaturated heterocyclic ring having one or two heteroatoms independently selected from nitrogen, oxygen, or sulfur. In certain embodiments, Ring D⁷ is tetrahydropyridyl. In some embodiments, Ring D⁷ is phenyl. In some embodiments, D⁷ is an optionally substituted 5-6 membered heteroaryl ring having 1-3 heteroatoms independently selected from nitrogen, oxygen, or sulfur. In certain embodiments, Ring D⁷ is pyridyl. In some embodiments, Ring D⁷ is an optionally substituted 3-7 membered saturated or partially unsaturated carbocyclic ring. In certain embodiments, Ring D⁷ is cyclohexyl. In certain embodiments, Ring D⁷ is absent. In some embodiments, Ring D⁷ is a 7-12 membered saturated or partially unsaturated bridged bicyclic ring having 0-4 heteroatoms independently selected from nitrogen, oxygen, or sulfur.

In certain embodiments, a provided compound of formula VII is:

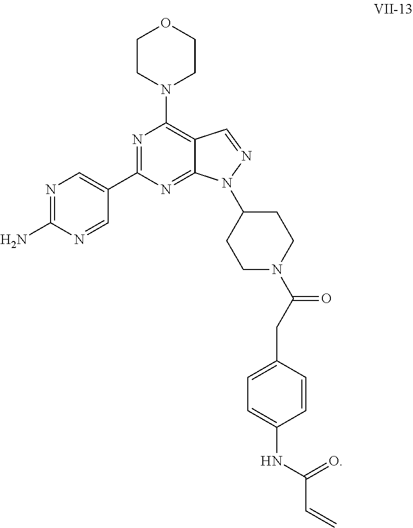

In certain embodiments, the present invention provides a compound of formula VIII:

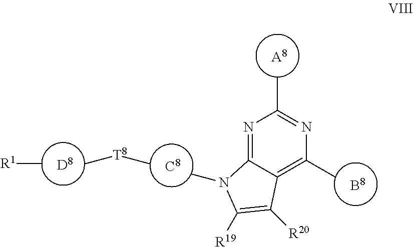

or a pharmaceutically acceptable salt thereof, wherein:
R¹ is a warhead group;
Ring A⁸ is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R¹⁹ and R²⁰ are independently R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
   two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B⁸ is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T⁸ is a covalent bond or a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C⁸ is an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; and
Ring D⁸ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

One of ordinary skill in the art will appreciate that when Ring D⁸ of formula VIII is absent, R¹ is directly attached to T⁸.

In certain embodiments, the Ring B⁸ group of formula VIII is an optionally substituted 8-10 membered bicyclic heteroaryl ring having 1-4 heteroatoms independently selected from nitrogen, oxygen, or sulfur. In some embodiments, Ring B⁸ is an optionally substituted 8-10 membered bicyclic heteroaryl ring having 2 nitrogen atoms. In some embodiments, Ring B⁸ is 1H-indazolyl, benzimidazolyl, or indolyl. In certain embodiments, Ring B⁸ is 1H-indazolyl. In certain embodiments, the Ring B⁸ group is substituted or unsubstituted phenyl. In certain embodiments, Ring B⁸ is substituted phenyl. In some embodiments, Ring B⁸ is a 5-6 membered heteroaryl ring having 1-4 heteroatoms independently selected from nitrogen, oxygen, or sulfur. In some embodiments, Ring B⁸ is an optionally substituted 5-6 membered heteroaryl ring having 1-2 nitrogen atoms. In certain embodiments, Ring B⁸ is pyridyl. In certain embodiments, Ring B⁸ is optionally substituted pyrimidinyl. In certain embodiments, Ring B⁸ is

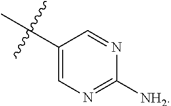

In certain embodiments, the Ring A⁸ group of formula VIII is an optionally substituted 5-6 membered saturated or partially unsaturated heterocyclic ring having one or two heteroatoms independently selected from nitrogen, oxygen, or sulfur. In some embodiments, Ring A⁸ is an optionally substituted 6-membered saturated or partially unsaturated heterocyclic ring having one or two heteroatoms independently selected from nitrogen, oxygen, or sulfur. In some embodiments, Ring A⁸ is optionally substituted morpholinyl. In certain embodiments, Ring A⁸ is unsubstituted morpholinyl. In some embodiments, Ring A⁸ is optionally substituted tetrahydropyranyl. In certain embodiments, A⁸ is:

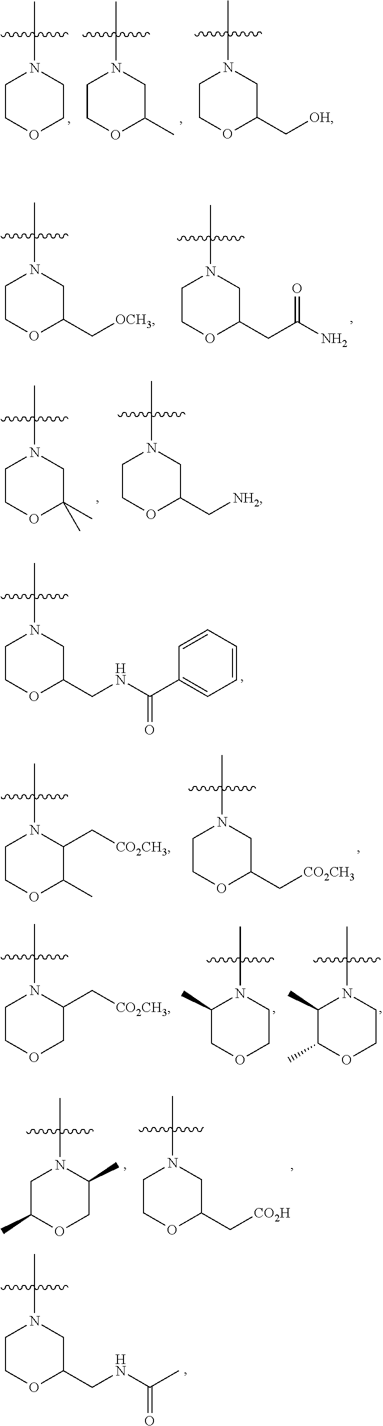

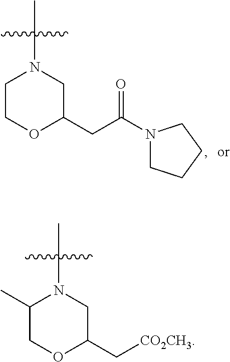

In certain embodiments, Ring A⁸ is an optionally substituted ring 5-15 membered saturated or partially unsaturated bridged bicyclic heterocyclic ring having at least one nitrogen, at least one oxygen, and optionally 1-2 additional heteroatoms independently selected from nitrogen, oxygen, or sulfur. In certain embodiments, Ring A⁸ is an optionally substituted ring 5-10 membered saturated or partially unsaturated bridged bicyclic heterocyclic ring having at least one nitrogen, at least one oxygen, and optionally 1-2 additional heteroatoms independently selected from nitrogen, oxygen, or sulfur. In certain embodiments, Ring A⁸ is a bridged, bicyclic morpholino group. In certain embodiments, A⁸ is an optionally substituted ring having the structure:

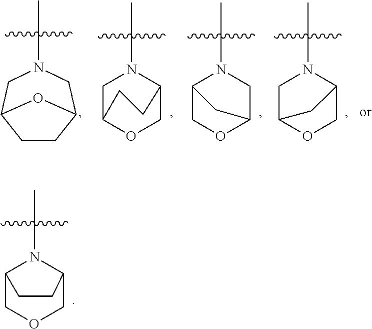

In certain embodiments, Ring A⁸ is of the formula:

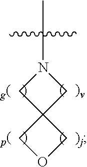

wherein:
v, j, p, and g are independently 1, 2, or 3.

In some embodiments, Ring A⁸ is an optionally substituted bicyclic (fused or spiro-fused) ring selected from:

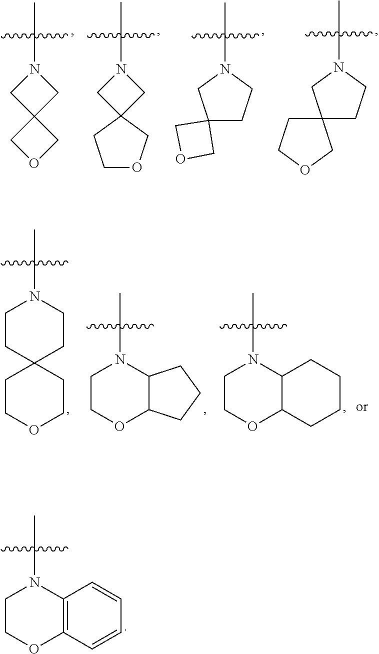

In certain embodiments, the T⁸ group of formula VIII is a bivalent, straight, saturated C₁₋₆ hydrocarbon chain. In some embodiments, T⁸ is a bivalent, straight, saturated C₁₋₃ hydrocarbon chain. In some embodiments, T⁸ is —CH₂—. In certain embodiments, T⁸ is a covalent bond. In certain embodiments, T⁸ is —C(O)—.

In certain embodiments, the Ring C⁸ group of formula VIII is an optionally substituted 6-membered saturated heterocyclic ring having one or two heteroatoms independently selected from nitrogen, oxygen, or sulfur. In some embodiments, Ring C⁸ is a piperazinyl or piperidinyl ring. In certain embodiments, Ring C⁸ is piperidinyl. In certain embodiments, Ring C⁸ is substituted with one or more oxo groups. In certain embodiments, Ring C⁸ is thiomorpholine optionally substituted with one or more oxo groups. In some embodiments, Ring C⁸ is an optionally substituted 6-membered partially unsaturated heterocyclic ring having one or two heteroatoms independently selected from nitrogen, oxygen, or sulfur. In certain embodiments, Ring C⁸ is tetrahydropyridyl. In some embodiments, Ring C⁸ is optionally substituted phenyl. In certain embodiments, Ring C⁸ is unsubstituted phenyl. In certain embodiments, Ring C⁸ is phenyl substituted with methyl. In certain embodiments, Ring C⁸ is

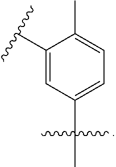

In some embodiments, C⁸ is an optionally substituted 5-6 membered heteroaryl ring having 1-3 heteroatoms independently selected from nitrogen, oxygen, or sulfur. In certain embodiments, Ring C⁸ is pyridyl. In some embodiments, Ring C⁸ is an optionally substituted 3-7 membered saturated or partially unsaturated carbocyclic ring. In certain embodiments, Ring C⁸ is cyclohexyl. In some embodiments, Ring C⁸ is a 7-12 membered saturated or partially unsaturated bridged bicyclic ring having 0-4 heteroatoms independently selected from nitrogen, oxygen, or sulfur.

In certain embodiments, the Ring D⁸ group of formula VIII is an optionally substituted 6-membered saturated heterocyclic ring having one or two heteroatoms independently selected from nitrogen, oxygen, or sulfur. In some embodiments, Ring D⁸ is a piperazinyl or piperidinyl ring. In certain embodiments, Ring D⁸ is piperidinyl. In certain embodiments, Ring D⁸ is substituted with one or more oxo groups. In certain embodiments, Ring D⁸ is thiomorpholine optionally substituted with one or more oxo groups. In certain embodiments, Ring D⁸ is

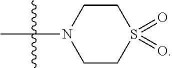

In some embodiments, Ring D⁸ is an optionally substituted 6-membered partially unsaturated heterocyclic ring having one or two heteroatoms independently selected from nitrogen, oxygen, or sulfur. In certain embodiments, Ring D⁸ is tetrahydropyridyl. In some embodiments, Ring D⁷ is phenyl. In some embodiments, D⁸ is an optionally substituted 5-6 membered heteroaryl ring having 1-3 heteroatoms independently selected from nitrogen, oxygen, or sulfur. In certain embodiments, Ring D⁸ is pyridyl. In some embodiments, Ring D⁸ is an optionally substituted 3-7 membered saturated or partially unsaturated carbocyclic ring. In certain embodiments, Ring D⁸ is cyclohexyl. In certain embodiments, Ring D⁸ is absent. In some embodiments, Ring D⁸ is a 7-12 membered saturated or partially unsaturated bridged bicyclic ring having 0-4 heteroatoms independently selected from nitrogen, oxygen, or sulfur.

In certain embodiments, the present invention provides a compound of formula IX:

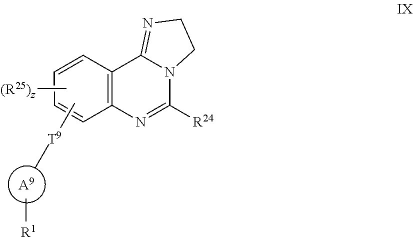

or a pharmaceutically acceptable salt thereof, wherein:
R¹ is a warhead group;
T⁹ is a covalent bond or a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring A⁹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
R²⁴ and R²⁵ are independently R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
   two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur; and
z is 0, 1, or 2.

It will be understood by one of ordinary skill in the art that when Ring A⁹ is absent, R¹ is directly attached to T⁹.

In some embodiments, R²⁴ of formula IX is R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂. In some embodiments, R²⁴ is —NRC(O)R, —NRC(O)N(R)₂, or —NRSO₂R. In certain embodiments, R²⁴ is R²⁴ is —NRC(O)R. In certain embodiments, R²⁴ is R²⁴ is —NHC(O)(pyridyl).

In some embodiments, R²⁵ of formula IX is R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂. In some embodiments, R²⁵ is —OR or —N(R)₂. In certain embodiments, R²⁵ is —OCH₃.

In certain embodiments, the T⁹ group of formula IX is a bivalent, straight, saturated C₁₋₆ hydrocarbon chain wherein 1-3 methylene units of T⁹ is replaced by —O—, —S—, —N(R)—C(O)—, 13 OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—. In some embodiments, T⁹ is a bivalent, straight, saturated C₅ hydrocarbon chain wherein 1-3 methylene units of T⁹ is replaced by —O—, —S—, —N(R)—C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—. In some embodiments, T⁹ is a bivalent, straight, saturated C₅ hydrocarbon chain wherein 3 methylene units of T⁹ is replaced by —O—, —N(R) or —C(O)—. In some embodiments, T⁹ is a bivalent, straight, saturated C₁₋₃ hydrocarbon chain wherein 1-3 methylene units of T⁹ is replaced by —O—, —N(R)—, or —C(O)—. In certain embodiments, T⁹ is —OCH₂CH₂NHC(O)—. In certain embodiments, T⁹ is a covalent bond. In certain embodiments, T⁹ is —C(O)—. In certain embodiments, T⁹ is —O—. In certain embodiments, T⁹ is —OCH₂CH₂—.

In some embodiments, Ring A⁹ of formula IX is an optionally substituted 6-membered heterocyclic ring having 1-2 nitrogens. In certain embodiments, Ring A⁹ is a piperidine ring. In certain embodiments, Ring A⁹ is a piperazine ring. In some embodiments, Ring A⁹ is an optionally substituted 6-membered heteroaryl ring having 1-2 nitrogens. In certain embodiments, Ring A⁹ is a pyridine ring. In certain embodiments, Ring A⁹ is a pyrimidine ring. In certain embodiments, Ring A⁹ is a pyrazine ring. In certain embodiments, Ring A⁹ is a pyridazine ring. In some embodiments, Ring A⁹ is optionally substituted phenyl. In some embodiments, Ring A⁹ is unsubstituted phenyl. In some embodiments, Ring A⁹ is an optionally substituted 8-10 membered bicyclic heteroaryl ring having 1-4 heteroatoms independently selected from nitrogen, oxygen, or sulfur. In certain embodiments, Ring A⁹ is a tetrahydroisoquinoline ring. In certain embodiments, Ring A⁹ is absent.

In some embodiments, a compound of formula IX is of formula IX-a:

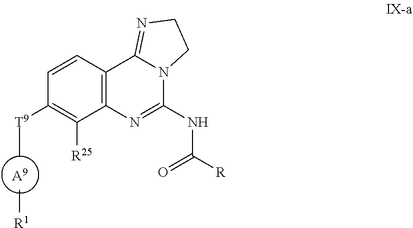

wherein R¹, T⁹, A⁹, R²⁵, and R are as defined above and described in classes and subclasses herein.

In certain embodiments, the present invention provides a compound of formula X:

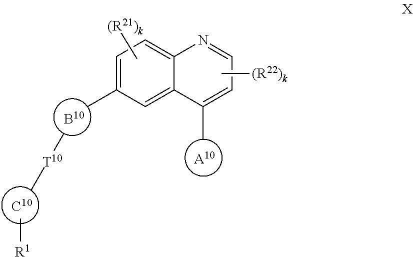

or a pharmaceutically acceptable salt thereof, wherein:
R¹ is a warhead group;
each R²¹ and R²² is independently —R″, halogen, —NO₂, —CN, —OR″, —SR″, —N(R″)₂, —C(O)R″, —CO₂R″, —C(O)C(O)R″, —C(O)CH₂C(O)R″, —S(O)R″, —S(O)₂R″, —C(O)N(R″)₂, —SO₂N(R″)₂, —OC(O)R″, —N(R″)C(O)R″, —N(R″)N(R″)₂, —N(R″)C(═NR″)N(R″)₂, —C(═NR″)N(R″)₂, —C═NOR″, —N(R″)C(O)N(R″)₂, —N(R″)SO₂N(R″)₂, —N(R″)SO₂R″, or —OC(O)N(R″)₂;
each R″ is independently hydrogen or an optionally substituted group selected from C₁₋₆ aliphatic, a 3-7 membered saturated or partially unsaturated carbocyclic ring, a 7-10 membered saturated or partially unsaturated bicyclic carbocyclic ring, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; or
   two R″ groups on the same nitrogen are taken together with the nitrogen to which they are attached to form an optionally substituted 5-8 membered saturated, partially unsaturated, or aromatic ring having 1-4 heteroatoms independently selected from nitrogen, oxygen, or sulfur;
each k is independently 0, 1, or 2;
Ring A¹⁰ is an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
Ring B¹⁰ is an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T¹⁰ is a covalent bond or a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring C¹⁰ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

It will be understood by one of ordinary skill in the art that when Ring C¹⁰ of formula X is absent, R¹ is directly attached to T¹⁰.

In some embodiments, Ring A¹⁰ of formulae X is an optionally substituted 6-membered heterocyclic ring having 1-2 nitrogens. In certain embodiments, Ring A¹⁰ is a piperidine ring. In certain embodiments, Ring A¹⁰ is a piperazine ring. In some embodiments, Ring A¹⁰ is an optionally substituted 6-membered heteroaryl ring having 1-2 nitrogens. In certain embodiments, Ring A¹⁰ is a pyridine ring. In certain embodiments, Ring A¹⁰ is a pyrimidine ring. In certain embodiments, Ring A¹⁰ is a pyrazine ring. In certain embodiments, Ring A¹⁰ is a pyridazine ring.

In some embodiments, Ring B¹⁰ of formulae X is an optionally substituted 6-membered heterocyclic ring having 1-2 nitrogens. In certain embodiments, Ring B¹⁰ is a piperidine ring. In certain embodiments, Ring B¹⁰ is a piperazine ring. In some embodiments, Ring B¹⁰ is an optionally substituted 6-membered heteroaryl ring having 1-2 nitrogens. In certain embodiments, Ring B¹⁰ is a pyridine ring. In certain embodiments, Ring B¹⁰ is a pyrimidine ring. In certain embodiments, Ring B¹⁰ is a pyrazine ring. In certain embodiments, Ring B¹⁰ is a pyridazine ring. In certain embodiments, Ring B¹⁰ is phenyl, pyridine, pyrimidine, pyrazine, or pyridazine substituted with an alkoxy group. In certain embodiments, Ring B¹⁰ is pyridine substituted with a methoxy group.

In certain embodiments, the T¹⁰ group of formula X is a bivalent, straight, saturated C₁₋₆ hydrocarbon chain. In some embodiments, T¹⁰ is a bivalent, straight, saturated C₁₋₃ hydrocarbon chain. In some embodiments, T¹⁰ is —CH₂—. In certain embodiments, T¹⁰ is a covalent bond. In certain embodiments, T¹⁰ is —C(O)—. In certain embodiments, T¹⁰ is —NHSO₂—. In certain embodiments, T¹⁰ is —SO₂—.

In certain embodiments, the Ring C¹⁰ group of formula X is an optionally substituted 6-membered saturated heterocyclic ring having one or two heteroatoms independently selected from nitrogen, oxygen, or sulfur. In some embodiments, Ring C¹⁰ is a piperazinyl or piperidinyl ring. In certain embodiments, Ring C¹⁰ is piperidinyl. In some embodiments, Ring C¹⁰ is an optionally substituted 6-membered partially unsaturated heterocyclic ring having one or two heteroatoms independently selected from nitrogen, oxygen, or sulfur. In certain embodiments, Ring C¹⁰ is tetrahydropyridyl. In some embodiments, Ring C¹⁰ is phenyl. In some embodiments, C¹⁰ is an optionally substituted 5-6 membered heteroaryl ring having 1-3 heteroatoms independently selected from nitrogen, oxygen, or sulfur. In certain embodiments, Ring C¹⁰ is pyridyl. In some embodiments, Ring C¹⁰ is an optionally substituted 3-7 membered saturated or partially unsaturated carbocyclic ring. In certain embodiments, Ring C¹⁰ is cyclohexyl. In some embodiments, Ring C¹⁰ is a 7-12 membered saturated or partially unsaturated bridged bicyclic ring having 0-4 heteroatoms independently selected from nitrogen, oxygen, or sulfur.

In certain embodiments, k of formulae X is O. In some embodiments, k is 1. In other embodiments, k is 2.

In certain embodiments, the present invention provides a compound of formula XI:

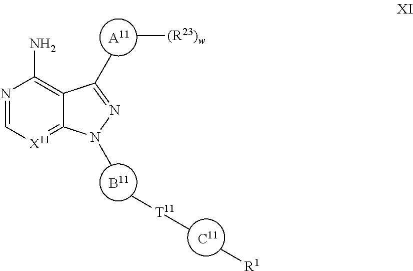

or a pharmaceutically acceptable salt thereof, wherein:
R¹ is a warhead group;
X¹¹ is CH or N;
Ring A¹¹ is an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
each R²³ is independently —R^{a}, halogen, —NO₂, —CN, —OR^{b}, —SR^{b}, —N(R^{b})₂, —C(O)R^{a}, —CO₂R^{a}, —C(O)C(O)R^{a}, —C(O)CH₂C(O)R^{a}, —S(O)R^{a}, —S(O)₂R^{a}, —C(O)N(R^{a})₂, —SO₂N(R^{a})₂, —OC(O)R^{a}, —N(R^{a})C(O)R^{a}, —N(R^{a})N(R^{a})₂, —N(R^{a})C(═NR^{a})N(R^{a})₂, —C(═NR^{a})N(R^{a})₂, —C═NOR^{a}, —N(R^{a})C(O)N(R^{a})₂, —N(R^{a})SO₂N(R^{a})₂, —N(R^{a})SO₂R^{a}, or —OC(O)N(R^{a})₂;
each R^{a} is independently hydrogen, C₁₋₆ aliphatic, phenyl, a 3-7 membered saturated or partially unsaturated carbocyclic ring, a 7-10 membered saturated or partially unsaturated bicyclic carbocyclic ring, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; or
   two R^{a} groups on the same nitrogen are taken together with the nitrogen to which they are attached to form an optionally substituted 5-8 membered saturated, partially unsaturated, or aromatic ring having 1-4 heteroatoms independently selected from nitrogen, oxygen, or sulfur;
each R^{b} is independently hydrogen, C₁₋₆ aliphatic, a 3-7 membered saturated or partially unsaturated carbocyclic ring, a 7-10 membered saturated or partially unsaturated bicyclic carbocyclic ring, a 4-7 membered saturated or partially unsaturated heterocyclic ring having 1-2 heteroatoms independently selected from nitrogen, oxygen, or sulfur, or a 7-10 membered saturated or partially unsaturated bicyclic heterocyclic ring having 1-3 heteroatoms independently selected from nitrogen, oxygen, or sulfur; or
   two R^{b} groups on the same nitrogen are taken together with the nitrogen to which they are attached to form an optionally substituted 5-8 membered saturated, partially unsaturated, or aromatic ring having 1-4 heteroatoms independently selected from nitrogen, oxygen, or sulfur;
w is 0, 1, or 2;
Ring B¹¹ is an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T¹¹ is a covalent bond or a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring C¹¹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

It will be understood by one of ordinary skill in the art that when Ring C¹¹ is absent, R¹ is directly attached to T¹¹.

In some embodiments, Ring A¹¹ of formula XI is phenyl optionally substituted with R²³. In certain embodiments, Ring A¹¹ is phenyl substituted with one or two R²³ groups. In certain embodiments, Ring A¹¹ is phenyl substituted with two R²³ groups. In certain embodiments, Ring A¹¹ is dimethoxyphenyl. In some embodiments, Ring A¹¹ is a 6-membered heterocyclic ring having 1-2 nitrogens optionally substituted with R²³. In certain embodiments, Ring A¹¹ is a piperidine ring. In certain embodiments, Ring A¹¹ is a piperazine ring. In some embodiments, Ring A¹¹ is a 6-membered heteroaryl ring having 1-2 nitrogens optionally substituted with R²³. In certain embodiments, Ring A¹¹ is a pyridine ring. In certain embodiments, Ring A¹¹ is a pyrimidine ring. In certain embodiments, Ring A¹¹ is a pyrazine ring. In certain embodiments, Ring A¹¹ is a pyridazine ring. In some embodiments, Ring A¹¹ is an 8-10 membered bicyclic heteroaryl ring having 1-4 heteroatoms independently selected from nitrogen, oxygen, or sulfur. In certain embodiments, Ring A¹¹ is 7-azaindole. In certain embodiments, Ring A¹¹ is indole optionally substituted with R²³. In certain embodiments, Ring A¹¹ is 6-hydroxyindole.

In some embodiments, Ring B¹¹ of formula XI is an optionally substituted 6-membered heterocyclic ring having 1-2 nitrogens. In certain embodiments, Ring B¹¹ is a piperidine ring. In certain embodiments, Ring B¹¹ is a piperazine ring. In some embodiments, Ring B¹¹ is an optionally substituted 6-membered heteroaryl ring having 1-2 nitrogens. In certain embodiments, Ring B¹¹ is a pyridine ring. In certain embodiments, Ring B¹¹ is a pyrimidine ring. In certain embodiments, Ring B¹¹ is a pyrazine ring. In certain embodiments, Ring B¹¹ is a pyridazine ring. In certain embodiments, Ring B¹¹ is phenyl.

In certain embodiments, the T¹¹ group of formula XI is a bivalent, straight, saturated C₁₋₆ hydrocarbon chain. In some embodiments, T¹¹ is a bivalent, straight, saturated C₁₋₃ hydrocarbon chain. In some embodiments, T¹¹ is —CH₂—. In certain embodiments, T¹¹ is a covalent bond. In certain embodiments, T¹¹ is —C(O)—.

In some embodiments, Ring C¹¹ of formula XI is an optionally substituted 6-membered heterocyclic ring having 1-2 nitrogens. In certain embodiments, Ring C¹¹ is a piperidine ring. In certain embodiments, Ring C¹¹ is a piperazine ring. In some embodiments, Ring C¹¹ is an optionally substituted 6-membered heteroaryl ring having 1-2 nitrogens. In certain embodiments, Ring C¹¹ is a pyridine ring. In certain embodiments, Ring C¹¹ is a pyrimidine ring. In certain embodiments, Ring C¹¹ is a pyrazine ring. In certain embodiments, Ring C¹¹ is a pyridazine ring. In certain embodiments, Ring C¹¹ is phenyl.

In certain embodiments, w of formulae XI is 0. In some embodiments, w is 1. In other embodiments, w is 2.

In certain embodiments, the present invention provides a compound of formula XII:

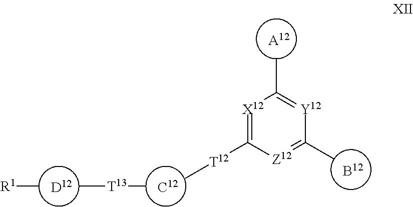

or a pharmaceutically acceptable salt thereof, wherein:
R¹ is a warhead group;
X¹² is CR²⁶ or N;
Y¹² is CR²⁷ or N;
Z¹² is CR²⁸ or N;
wherein at least one of X¹², Y¹², and Z¹² is N;
Ring A¹² is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R²⁶, R²⁷, and R²⁸ are independently R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
   two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B¹² is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T¹² is a covalent bond or a bivalent straight or branched, saturated or unsaturated C₁₋₆ hydrocarbon chain wherein one or more methylene units of T¹² are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C¹² is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged or spiro bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T¹³ is a covalent bond or a bivalent straight or branched, saturated or unsaturated C₁₋₆ hydrocarbon chain wherein one or more methylene units of T¹³ are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring D¹² is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

It will be understood by one of ordinary skill in the art that when Ring C¹² of formula XII is absent, T¹³ is directly attached to T¹². It will be further understood that when Ring D¹² is absent, R¹ is directly attached to T¹³.

In certain embodiments, the Ring B¹² group of formula XII is an optionally substituted 8-10 membered bicyclic heteroaryl ring having 1-4 heteroatoms independently selected from nitrogen, oxygen, or sulfur. In some embodiments, Ring B¹² is an optionally substituted 8-10 membered bicyclic heteroaryl ring having 2 nitrogen atoms. In some embodiments, Ring B¹² is 1H-indazolyl, benzimidazolyl, or indolyl. In certain embodiments, Ring B¹² is 1H-indazolyl. In certain embodiments, the Ring B¹² group is substituted or unsubstituted phenyl. In certain embodiments, Ring B¹² is substituted phenyl. In certain embodiments, Ring B¹² is phenol. In some embodiments, Ring B¹² is a 5-6 membered heteroaryl ring having 1-4 heteroatoms independently selected from nitrogen, oxygen, or sulfur. In some embodiments, Ring B¹² is an optionally substituted 5-6 membered heteroaryl ring having 1-2 nitrogen atoms. In certain embodiments, Ring B¹² is pyridyl. In certain embodiments, Ring B¹² is optionally substituted pyrimidinyl. In certain embodiments, Ring B¹² is

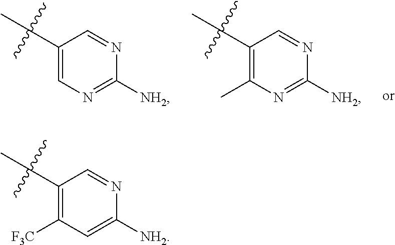

In certain embodiments, the Ring A¹² group of formula XII is an optionally substituted 5-6 membered saturated or partially unsaturated heterocyclic ring having one or two heteroatoms independently selected from nitrogen, oxygen, or sulfur. In some embodiments, Ring A¹² is an optionally substituted 6-membered saturated or partially unsaturated heterocyclic ring having one or two heteroatoms independently selected from nitrogen, oxygen, or sulfur. In some embodiments, Ring A¹² is optionally substituted morpholinyl. In certain embodiments, Ring A¹² is unsubstituted morpholinyl. In some embodiments, Ring A¹² is optionally substituted tetrahydropyranyl. In certain embodiments, A¹² is:

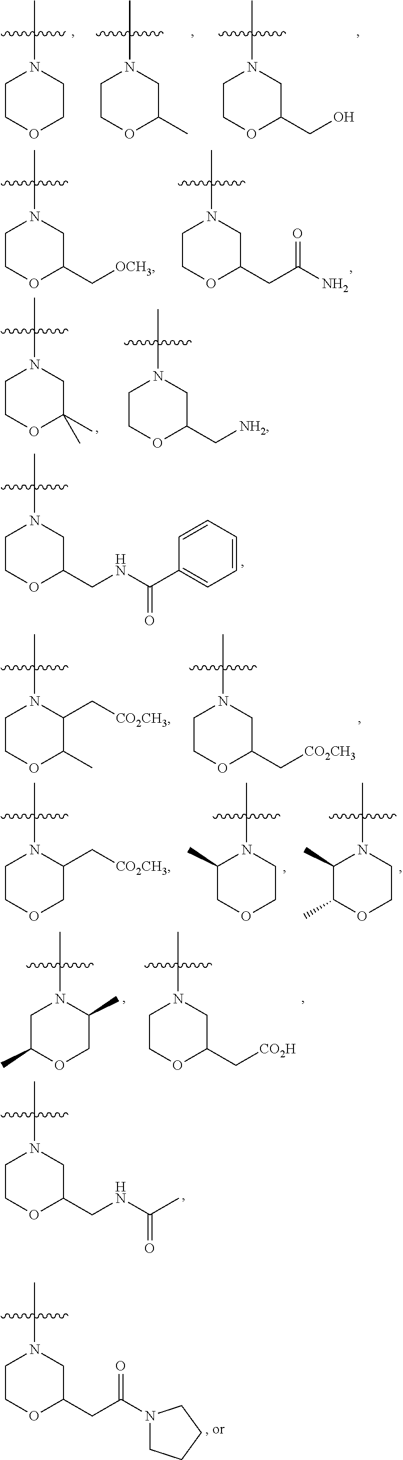

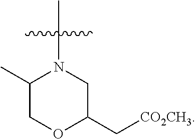

In certain embodiments, Ring A¹² is an optionally substituted ring 5-15 membered saturated or partially unsaturated bridged bicyclic heterocyclic ring having at least one nitrogen, at least one oxygen, and optionally 1-2 additional heteroatoms independently selected from nitrogen, oxygen, or sulfur. In certain embodiments, Ring A¹² is an optionally substituted ring 5-10 membered saturated or partially unsaturated bridged bicyclic heterocyclic ring having at least one nitrogen, at least one oxygen, and optionally 1-2 additional heteroatoms independently selected from nitrogen, oxygen, or sulfur. In certain embodiments, Ring A¹² is a bridged, bicyclic morpholino group. In certain embodiments, A¹² is an optionally substituted ring having the structure:

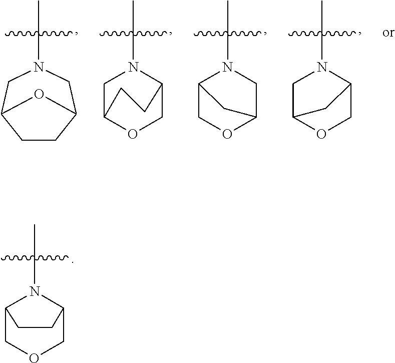

In certain embodiments, Ring A¹² is of the formula:

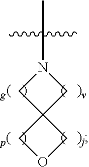

wherein:
v, j, p, and g are independently 1, 2, or 3.

In some embodiments, Ring A¹² is an optionally substituted bicyclic (fused or spiro-fused) ring selected from:

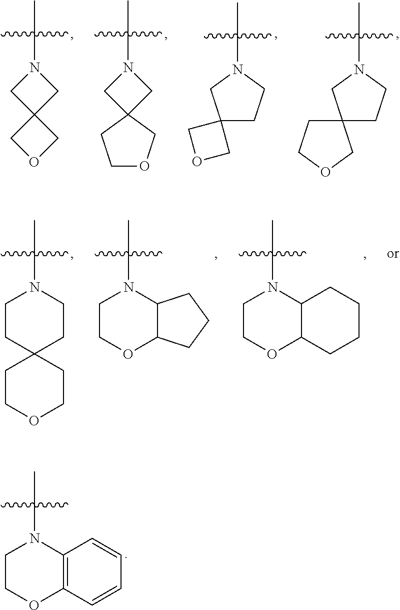

In certain embodiments, the T¹² group of formula XII is a bivalent, straight, saturated C₁₋₆ hydrocarbon chain. In some embodiments, T¹² is a bivalent, straight, saturated C₁₋₃ hydrocarbon chain. In some embodiments, T¹² is —CH₂— or —CH₂CH₂—. In other embodiments, T¹² is —C(O)—. In certain embodiments, T¹² is —C≡C— or —CH₂C≡C—. In certain embodiments, T¹² is a covalent bond.

In certain embodiments, the Ring C¹² group of formula XII is an optionally substituted 6-membered saturated heterocyclic ring having one or two heteroatoms independently selected from nitrogen, oxygen, or sulfur. In some embodiments, Ring C¹² is a piperazinyl or piperidinyl ring. In some embodiments, Ring C¹² is an optionally substituted 6-membered partially unsaturated heterocyclic ring having one or two heteroatoms independently selected from nitrogen, oxygen, or sulfur. In certain embodiments, Ring C¹² is tetrahydropyridyl. In some embodiments, Ring C¹² is phenyl. In some embodiments, Ring C¹² is an optionally substituted 3-7 membered saturated or partially unsaturated carbocyclic ring. In certain embodiments, Ring C¹² is cyclohexyl. In certain embodiments, Ring C¹² is absent. In some embodiments, Ring C¹² is a 7-12 membered saturated or partially unsaturated bridged or spiro bicyclic ring having 0-4 heteroatoms independently selected from nitrogen, oxygen, or sulfur.

In certain embodiments, the T¹³ group of formula XII is a bivalent, straight, saturated C₁₋₆ hydrocarbon chain. In some embodiments, T¹³ is a bivalent, straight, saturated C₁₋₃ hydrocarbon chain. In some embodiments, T¹³ is —CH₂— or —CH₂CH₂—. In certain embodiments, T¹³ is —C(O)—. In certain embodiments, T¹³ is a covalent bond.

In certain embodiments, the Ring D¹² group of formula XII is an optionally substituted 6-membered saturated heterocyclic ring having one or two heteroatoms independently selected from nitrogen, oxygen, or sulfur. In some embodiments, Ring D¹² is a piperazinyl or piperidinyl ring. In some embodiments, Ring D¹² is an optionally substituted 6-membered partially unsaturated heterocyclic ring having one or two heteroatoms independently selected from nitrogen, oxygen, or sulfur. In certain embodiments, Ring D¹² is tetrahydropyridyl. In some embodiments, Ring D¹² is phenyl. In some embodiments, Ring D¹² is an optionally substituted 3-7 membered saturated or partially unsaturated carbocyclic ring. In certain embodiments, Ring D¹² is cyclohexyl. In certain embodiments, Ring D¹² is absent. In some embodiments, Ring D¹² is a 7-12 membered saturated or partially unsaturated bridged bicyclic ring having 0-4 heteroatoms independently selected from nitrogen, oxygen, or sulfur.

In certain embodiments, a compound of formula XII is of formula XII-a:

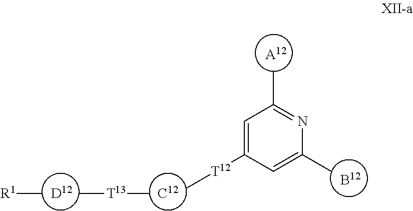

wherein Ring A¹², Ring B¹², T¹², Ring C¹², T¹³, and Ring D¹² are as defined above and described in classes and subclasses herein.

It will be understood by one of ordinary skill in the art that when Ring C¹² of formula XII-a is absent, T¹³ is directly attached to T¹². It will be further understood that when Ring D¹² is absent, R¹ is directly attached to T¹³.

In certain embodiments, the Ring B¹² group of formula XII-a is an optionally substituted 8-10 membered bicyclic heteroaryl ring having 1-4 heteroatoms independently selected from nitrogen, oxygen, or sulfur. In some embodiments, Ring B¹² is an optionally substituted 8-10 membered bicyclic heteroaryl ring having 2 nitrogen atoms. In some embodiments, Ring B¹² is 1H-indazolyl, benzimidazolyl, or indolyl. In certain embodiments, Ring B¹² is 1H-indazolyl. In certain embodiments, the Ring B¹² group is substituted or unsubstituted phenyl. In certain embodiments, Ring B¹² is substituted phenyl. In certain embodiments, Ring B¹² is phenol. In some embodiments, Ring B¹² is a 5-6 membered heteroaryl ring having 1-4 heteroatoms independently selected from nitrogen, oxygen, or sulfur. In some embodiments, Ring B¹² is an optionally substituted 5-6 membered heteroaryl ring having 1-2 nitrogen atoms. In certain embodiments, Ring B¹² is pyridyl. In certain embodiments, Ring B¹² is optionally substituted pyrimidinyl. In certain embodiments, Ring B¹² is

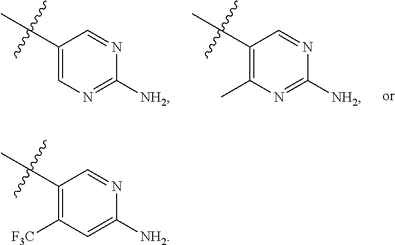

In certain embodiments, the Ring A¹² group of formula XII-a is an optionally substituted 5-6 membered saturated or partially unsaturated heterocyclic ring having one or two heteroatoms independently selected from nitrogen, oxygen, or sulfur. In some embodiments, Ring A¹² is an optionally substituted 6-membered saturated or partially unsaturated heterocyclic ring having one or two heteroatoms independently selected from nitrogen, oxygen, or sulfur. In some embodiments, Ring A¹² is optionally substituted morpholinyl. In certain embodiments, Ring A¹² is unsubstituted morpholinyl. In some embodiments, Ring A¹² is optionally substituted tetrahydropyranyl. In certain embodiments, A¹² is:

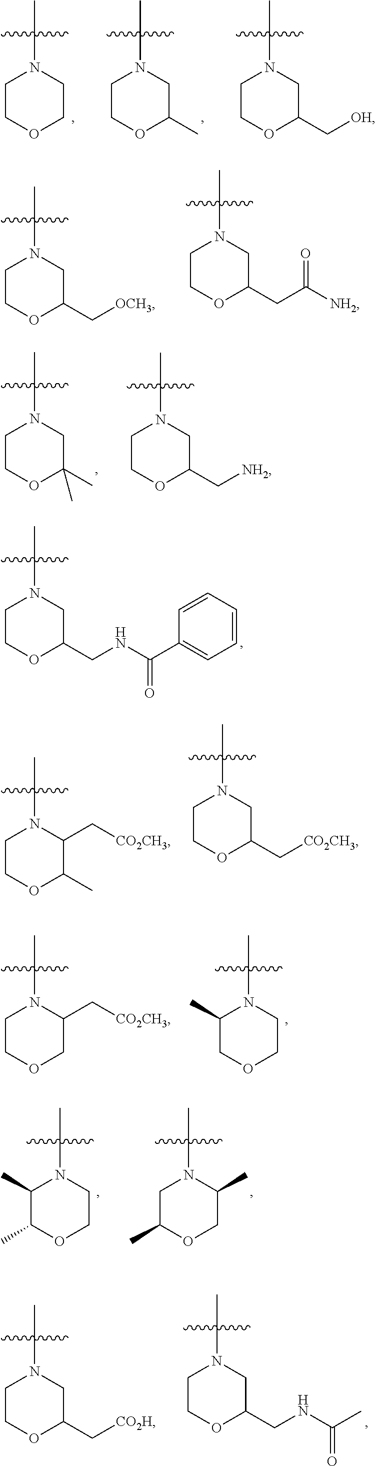

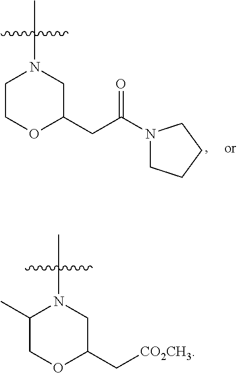

In certain embodiments, Ring A¹² is an optionally substituted ring 5-15 membered saturated or partially unsaturated bridged bicyclic heterocyclic ring having at least one nitrogen, at least one oxygen, and optionally 1-2 additional heteroatoms independently selected from nitrogen, oxygen, or sulfur. In certain embodiments, Ring A¹² is an optionally substituted ring 5-10 membered saturated or partially unsaturated bridged bicyclic heterocyclic ring having at least one nitrogen, at least one oxygen, and optionally 1-2 additional heteroatoms independently selected from nitrogen, oxygen, or sulfur. In certain embodiments, Ring A¹² is a bridged, bicyclic morpholino group. In certain embodiments, A¹² is an optionally substituted ring having the structure:

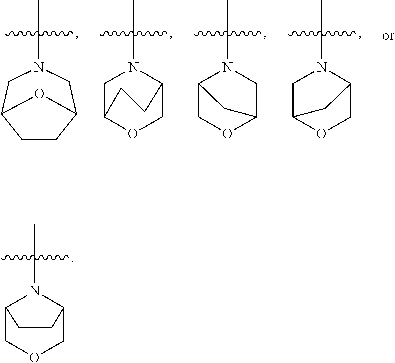

In certain embodiments, Ring A¹² is of the formula:

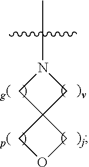

wherein:
v, j, p, and g are independently 1, 2, or 3.

In some embodiments, Ring A¹² is an optionally substituted ring having the structure:

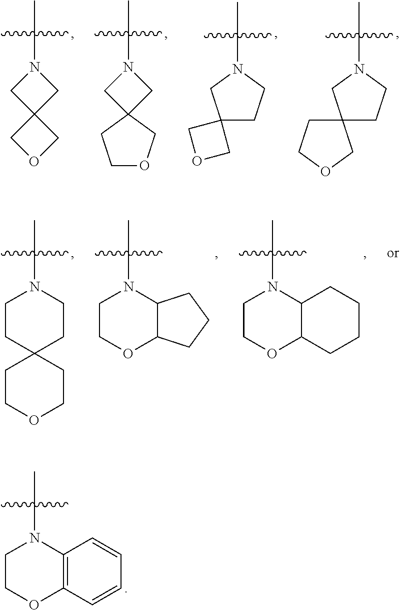

In certain embodiments, the T¹² group of either of formula II-a or II-b is a bivalent, straight, saturated C₁₋₆ hydrocarbon chain. In some embodiments, T¹² is a bivalent, straight, saturated C₁₋₃ hydrocarbon chain. In some embodiments, T¹² is —CH₂— or —CH₂CH₂—. In other embodiments, T¹² is —C(O)—. In certain embodiments, T¹² is —C≡C— or —CH₂C≡C—. In certain embodiments, T¹² is a covalent bond. In some embodiments, T¹² is a covalent bond, methylene, or a C₂₋₄ hydrocarbon chain wherein one methylene unit of T¹² is replaced by —C(O)NH—. In certain embodiments, T¹² is a C₃ hydrocarbon chain wherein one methylene unit of T¹² is replaced by —C(O)NH—.

In certain embodiments, the Ring C¹² group of either of formula II-a or II-b is an optionally substituted 6-membered saturated heterocyclic ring having one or two heteroatoms independently selected from nitrogen, oxygen, or sulfur. In some embodiments, Ring C¹² is a piperazinyl or piperidinyl ring. In some embodiments, Ring C¹² is an optionally substituted 6-membered partially unsaturated heterocyclic ring having one or two heteroatoms independently selected from nitrogen, oxygen, or sulfur. In certain embodiments, Ring C¹² is tetrahydropyridyl. In some embodiments, Ring C¹² is phenyl. In some embodiments, Ring C¹² is an optionally substituted 3-7 membered saturated or partially unsaturated carbocyclic ring. In certain embodiments, Ring C¹² is cyclohexyl. In certain embodiments, Ring C¹² is absent. In some embodiments, Ring C¹² is a 7-12 membered saturated or partially unsaturated bridged or spiro bicyclic ring having 0-4 heteroatoms independently selected from nitrogen, oxygen, or sulfur.

In certain embodiments, the T¹³ group of either of formula II-a or II-b is a bivalent, straight, saturated C₁₋₆ hydrocarbon chain. In some embodiments, T¹³ is a bivalent, straight, saturated C₁₋₃ hydrocarbon chain. In some embodiments, T¹³ is —CH₂— or —CH₂CH₂—. In certain embodiments, T¹³ is —C(O)—. In certain embodiments, T¹³ is a covalent bond.

In certain embodiments, the Ring D¹² group of either of formula II-a or II-b is an optionally substituted 6-membered saturated heterocyclic ring having one or two heteroatoms independently selected from nitrogen, oxygen, or sulfur. In some embodiments, Ring D¹² is a piperazinyl or piperidinyl ring. In some embodiments, Ring D¹² is an optionally substituted 6-membered partially unsaturated heterocyclic ring having one or two heteroatoms independently selected from nitrogen, oxygen, or sulfur. In certain embodiments, Ring D¹² is tetrahydropyridyl. In some embodiments, Ring D¹² is phenyl. In some embodiments, Ring D¹² is an optionally substituted 3-7 membered saturated or partially unsaturated carbocyclic ring. In certain embodiments, Ring D¹² is cyclohexyl. In certain embodiments, Ring D¹² is absent. In some embodiments, Ring D¹² is a 7-12 membered saturated or partially unsaturated bridged bicyclic ring having 0-4 heteroatoms independently selected from nitrogen, oxygen, or sulfur.

In certain embodiments, a compound of formula XII-a is of formula XII-a-i:

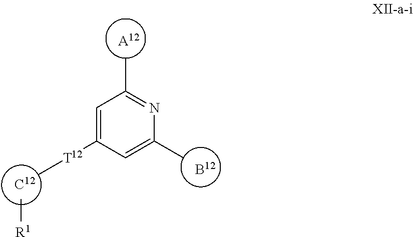

wherein Ring A¹², Ring B¹², T¹², Ring C¹², and R¹ are as defined above and described in classes and subclasses herein.

In certain embodiments, a compound of formula XII-a is of formula XII-a-ii:

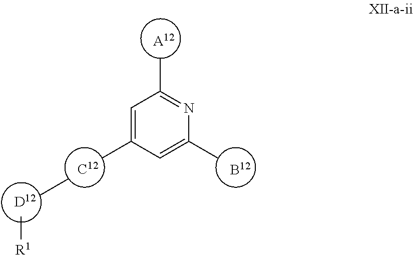

wherein Ring A¹², Ring B¹², Ring C¹², Ring D¹², and R¹ are as defined above and described in classes and subclasses herein.

In certain embodiments, a compound of formula XII-a is of formula XII-a-iii:

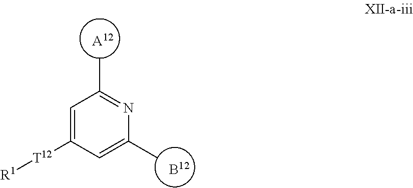

wherein Ring A¹², Ring B¹², T¹², and R¹ are as defined above and described in classes and subclasses herein.

In certain embodiments, a compound of formula XII is of formula XII-b:

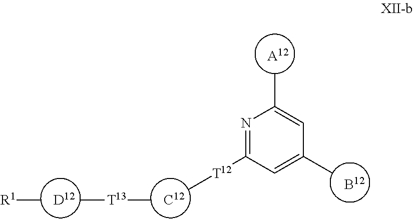

wherein Ring A¹², Ring B¹², T¹², Ring C¹², T¹³, Ring D¹², and R¹ are as defined above and described in classes and subclasses herein.

In certain embodiments, a compound of formula XII-b is of formula XII-b-i:

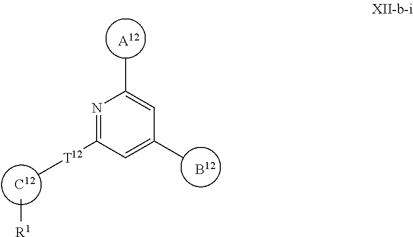

wherein Ring A¹², Ring B¹², T¹², Ring C¹², and R¹ are as defined above and described in classes and subclasses herein.

In certain embodiments, a compound of formula XII is of formula XII-c or XII-d:

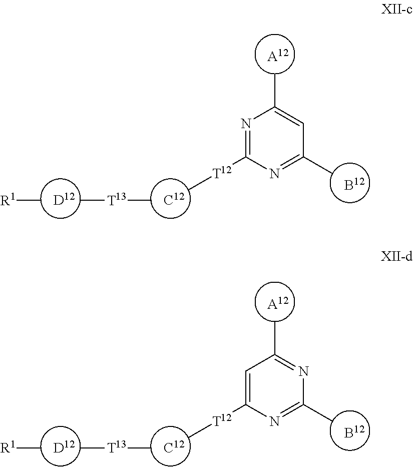

wherein Ring A¹², Ring B¹², T¹², Ring C¹², T¹³, Ring D¹², and R¹ are as defined above and described in classes and subclasses herein.

In certain embodiments, a compound of formula XII-c or XII-d is of formula XII-c-i or XII-d-i:

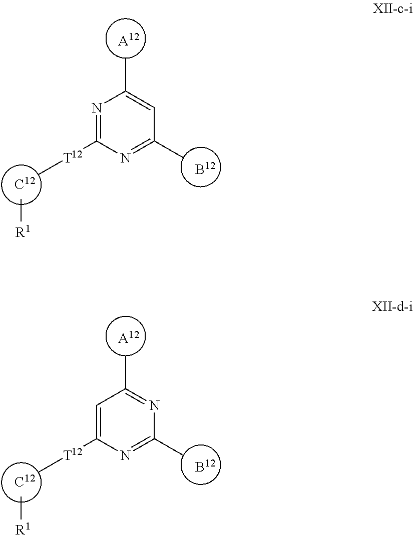

wherein Ring A¹², Ring B¹², T¹², Ring C¹², and R¹ are as defined above and described in classes and subclasses herein.

In certain embodiments, a compound of formula XII is of formula XII-e:

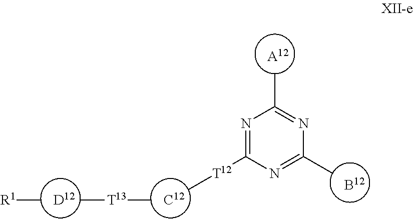

wherein Ring A¹², Ring B¹², T¹², Ring C¹², T¹³, Ring D¹², and R¹ are as defined above and described in classes and subclasses herein.

In certain embodiments, a compound of formula XII-e is of formula XII-e-i:

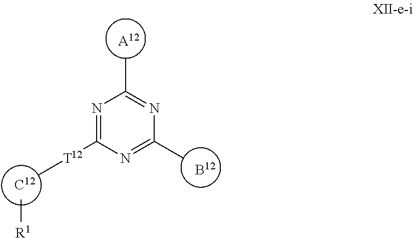

wherein Ring A¹², Ring B¹², T¹², Ring C¹², and R¹ are as defined above and described in classes and subclasses herein.

In some embodiments, a provided compound of formula XII-a, XII-b, XII-c, XII-d, or XII-e has one or more, more than one, or all of the features selected from:

a1) R¹ is selected from those embodiments described herein;
b1) Ring A¹² is selected from those embodiments described for formulae XII-a, XII-b, XII-c, XII-d, and XII-e, above;
c1) Ring B¹² is selected from those embodiments described for formulae XII-a, XII-b, XII-c, XII-d, and XII-e, above;
d1) T¹² is selected from those embodiments described for formulae XII-a, XII-b, XII-c, XII-d, and XII-e, above;
e1) Ring C¹² is selected from those embodiments described for formulae XII-a, XII-b, XII-c, XII-d, ad XII-e, above;
f1) T¹³ is selected from those embodiments described for formulae XII-a, XII-b, XII-c, XII-d, and XII-e, above; and
g1) Ring D¹² is selected from those embodiments described for formulae XII-a, XII-b, XII-c, XII-d, and XII-e, above.

In some embodiments,

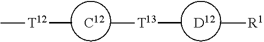

of formula XII-a, XII-b, XII-c, XII-d, or XII-e is

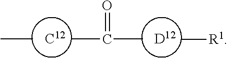

In some embodiments,

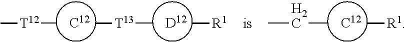

In some embodiments,

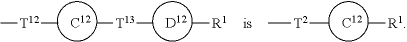

In some embodiments, a provided compound of formula XII-a, XII-b, XII-c, XII-d, or XII-e has one or more, more than one, or all of the features selected from:

a2) Ring A¹² is optionally substituted morpholinyl;
b2) Ring B¹² is an optionally substituted 8-10 membered bicyclic heteroaryl ring having 1-2 nitrogen atoms, optionally substituted phenyl, or an optionally substituted 5-6 membered heteroaryl ring having 1-2 nitrogen atoms;
c2)

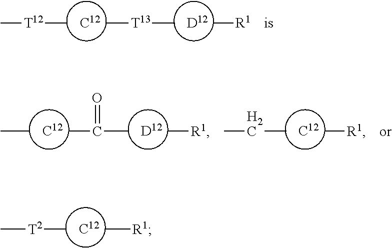

and
d2)

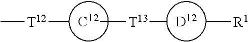

comprises a spacer group having about 9 to about 11 atoms. In some embodiments, a provided compound of formula XII-a, XII-b, XII-c, XII-d, or XII-e has one or more, more than one, or all of the features selected from: a2), b2), c2), and d2) described above, and e2) R¹ is selected from those embodiments described herein.

In some embodiments, a provided compound of formula XII-a, XII-b, XII-c, XII-d, or XII-e has one or more, more than one, or all of the features selected from:

a3) Ring A¹² is optionally substituted morpholinyl;
b3) Ring B¹² is an optionally substituted group selected from indazolyl, aminopyrimidinyl, or phenol;
c3)

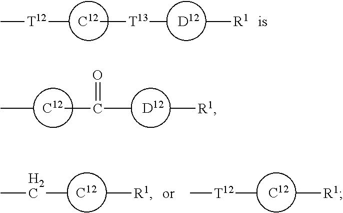

and
d3)

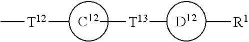

comprises a spacer group as defined herein having about 9 to about 11 atoms. In some embodiments, a provided compound of formula XII-a, XII-b, XII-c, XII-d, or XII-e has one or more, more than one, or all of the features selected from: a3), b3), c3), and d3) described above, and e3) R¹ is selected from those embodiments described herein.

In some embodiments, a provided compound of formula XII-a, XII-b, XII-c, XII-d, or XII-e has one or more, more than one, or all of the features selected from:

a4) Ring A¹² is optionally substituted morpholinyl;
b4) Ring B¹² is an optionally substituted 8-10 membered bicyclic heteroaryl ring having 1-2 nitrogen atoms, optionally substituted phenyl, or an optionally substituted 5-6 membered heteroaryl ring having 1-2 nitrogen atoms;
c4) T¹² is a covalent bond, methylene, or a C₂₋₄ hydrocarbon chain wherein one methylene unit of T¹² is replaced by —C(O)NH—;
d4) Ring C¹² is phenyl, or an optionally substituted 6-membered saturated, partially unsaturated, or aromatic heterocyclic ring having 1-2 nitrogens;
e4) T¹³ is a covalent bond, —C(O)—; and
f4) Ring D¹² is absent or phenyl.
In some embodiments, a provided compound of formula XII-a, XII-b, XII-c, XII-d, or XII-e has one or more, more than one, or all of the features selected from: a4), b4), c4), d4), e4), and f4) described above, and g4) R¹ is selected from those embodiments described herein.

In some embodiments, a provided compound of formula XII-a, XII-b, XII-c, XII-d, or XII-e has one or more, more than one, or all of the features selected from:

a5) Ring A¹² is optionally substituted morpholinyl;
b5) Ring B¹² is an optionally substituted group selected from indazolyl, phenol, or aminopyrimidine;
c5) T¹² is a covalent bond, methylene, or a C₃ hydrocarbon chain wherein one methylene unit of T¹² is replaced by —C(O)NH—;
d5) Ring C¹² is phenyl, piperazinyl, piperidinyl, or tetrahydropyridyl;
e5) T¹³ is a covalent bond or —C(O)—; and
f5) Ring D¹² is absent or phenyl.
In some embodiments, a provided compound of formula XII-a, XII-b, XII-c, XII-d, or XII-e has one or more, more than one, or all of the features selected from: a5), b5), c5), d5), e5), and f5) described above, and g5) R¹ is selected from those embodiments described herein.

In certain embodiments, a provided compound of formula XII-a, XII-b, XII-c, XII-d, or XII-e has one of the following structures:

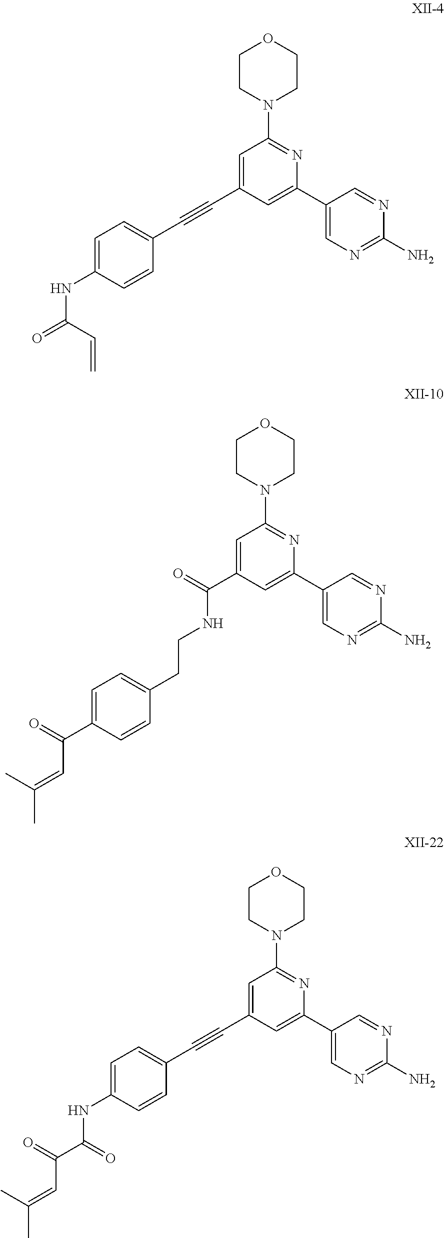

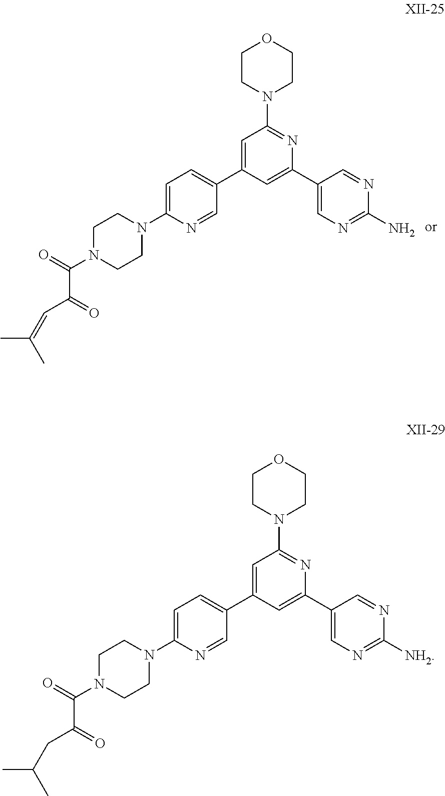

As defined generally above, the R¹ group of formulae I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, and XII-e is a warhead group. In certain embodiments, R¹ is -L-Y, wherein:
L is a covalent bond or a bivalent C₁₋₈ saturated or unsaturated, straight or branched, hydrocarbon chain, wherein one, two, or three methylene units of L are optionally and independently replaced by cyclopropylene, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—;
Y is hydrogen, C₁₋₆ aliphatic optionally substituted with oxo, halogen, NO₂, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with 1-4 R^{e} groups; and
each R^{e} is independently selected from -Q-Z, oxo, NO₂, halogen, CN, a suitable leaving group, or a C₁₋₆ aliphatic optionally substituted with oxo, halogen, NO₂, or CN, wherein:
   Q is a covalent bond or a bivalent C₁₋₆ saturated or unsaturated, straight or branched, hydrocarbon chain, wherein one or two methylene units of Q are optionally and independently replaced by —N(R)—S—, —O—, —C(O)—, —OC(O)—, —C(O)O—, —SO—, or —SO₂—, —N(R)C(O)—, —C(O)N(R)—, —N(R)SO₂—, or —SO₂N(R)—; and
   Z is hydrogen or C₁₋₆ aliphatic optionally substituted with oxo, halogen, NO₂, or CN.

In certain embodiments, L is a covalent bond.

In certain embodiments, L is a bivalent C₁₋₈ saturated or unsaturated, straight or branched, hydrocarbon chain. In certain embodiments, L is —CH₂—.

In certain embodiments, L is a covalent bond, —CH₂—, —NH—, —CH₂NH—, —NHCH₂—, —NHC(O)—, —NHC(O)CH₂OC(O)—, —CH₂NHC(O)—, —NHSO₂—, —NHSO₂CH₂—, —NHC(O)CH₂OC(O)—, or —SO₂NH—.

In certain embodiments, L is a bivalent C₁₋₈ hydrocarbon chain wherein at least one methylene unit of L is replaced by —C(O)—. In certain embodiments, L is a bivalent C₁₋₈ hydrocarbon chain wherein at least two methylene units of L are replaced by —C(O)—. In some embodiments, L is —C(O)CH₂CH₂C(O)—, —C(O)CH₂NHC(O)—, —C(O)CH₂NHC(O)CH₂CH₂C(O)—, or —C(O)CH₂CH₂CH₂NHC(O)CH₂CH₂C(O)—.

In certain embodiments, L is a bivalent C₁₋₈ hydrocarbon chain wherein at least one methylene unit of L is replaced by —S(O)₂—. In certain embodiments, L is a bivalent C₁₋₈ hydrocarbon chain wherein at least one methylene unit of L is replaced by —S(O)₂— and at least one methylene unit of L is replaced by —C(O)—. In certain embodiments, L is a bivalent C₁₋₈ hydrocarbon chain wherein at least one methylene unit of L is replaced by —S(O)₂— and at least two methylene units of L are replaced by —C(O)—. In some embodiments, L is —S(O)₂CH₂CH₂NHC(O)CH₂CH₂C(O)— or —S(O)₂CH₂CH₂NHC(O)—.

In some embodiments, L is a bivalent C₂₋₈ straight or branched, hydrocarbon chain wherein L has at least one double bond and one or two additional methylene units of L are optionally and independently replaced by —NRC(O)—, —C(O)NR—, —N(R)SO₂—, —SO₂N(R)—, —S—, —S(O)—, —SO₂—, —OC(O)—, —C(O)O—, cyclopropylene, —O—, —N(R)—, or —C(O)—.

In certain embodiments, L is a bivalent C₂₋₈ straight or branched, hydrocarbon chain wherein L has at least one double bond and at least one methylene unit of L is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L are optionally and independently replaced by cyclopropylene, —O—, —N(R)—, or —C(O)—.

In some embodiments, L is a bivalent C₂₋₈ straight or branched, hydrocarbon chain wherein L has at least one double bond and at least one methylene unit of L is replaced by —C(O)—, and one or two additional methylene units of L are optionally and independently replaced by cyclopropylene, —O—, —N(R)—, or —C(O)—.

As described above, in certain embodiments, L is a bivalent C₂₋₈ straight or branched, hydrocarbon chain wherein L has at least one double bond. One of ordinary skill in the art will recognize that such a double bond may exist within the hydrocarbon chain backbone or may be “exo” to the backbone chain and thus forming an alkylidene group. By way of example, such an L group having an alkylidene branched chain includes —CH₂C(═CH₂)CH₂—. Thus, in some embodiments, L is a bivalent C₂₋₈ straight or branched, hydrocarbon chain wherein L has at least one alkylidenyl double bond. Exemplary L groups include —NHC(O)C(═CH₂)CH₂—.

In certain embodiments, L is a bivalent C₂₋₈ straight or branched, hydrocarbon chain wherein L has at least one double bond and at least one methylene unit of L is replaced by —C(O)—. In certain embodiments, L is —C(O)CH═CH(CH₃)—, —C(O)CH═CHCH₂NH(CH₃)—, —C(O)CH═CH(CH₃)—, —C(O)CH═CH—, —CH₂C(O)CH═CH—, —CH₂C(O)CH═CH(CH₃)—, —CH₂CH₂C(O)CH═CH—, —CH₂CH₂C(O)CH═CHCH₂—, —CH₂CH₂C(O)CH═CHCH₂NH(CH₃)—, or —CH₂CH₂C(O)CH═CH(CH₃)—, or —CH(CH₃)OC(O)CH═CH—.

In certain embodiments, L is a bivalent C₂₋₈ straight or branched, hydrocarbon chain wherein L has at least one double bond and at least one methylene unit of L is replaced by —OC(O)—.

In some embodiments, L is a bivalent C₂₋₈ straight or branched, hydrocarbon chain wherein L has at least one double bond and at least one methylene unit of L is replaced by —NRC(O)—, —C(O)NR—, —N(R)SO₂—, —SO₂N(R)—, —S—, —S(O)—, —SO₂—, —OC(O)—, or —C(O)O—, and one or two additional methylene units of L are optionally and independently replaced by cyclopropylene, —O—, —N(R)—, or —C(O)—. In some embodiments, L is —CH₂OC(O)CH═CHCH₂—, —CH₂—OC(O)CH═CH—, or —CH(CH═CH₂)OC(O)CH═CH—.

In certain embodiments, L is —NRC(O)CH═CH—, —NRC(O)CH═CHCH₂N(CH₃)—, —NRC(O)CH═CHCH₂O—, —CH₂NRC(O)CH═CH—, —NRSO₂CH═CH—, —NRSO₂CH═CHCH₂—, —NRC(O)(C═N₂)C(O)—, —NRC(O)CH═CHCH₂N(CH₃)—, —NRSO₂CH═CH—, —NRSO₂CH═CHCH₂—, —NRC(O)CH═CHCH₂O—, —NRC(O)C(═CH₂)CH₂—, —CH₂NRC(O)—, —CH₂NRC(O)CH═CH—, —CH₂CH₂NRC(O)—, or —CH₂NRC(O)cyclopropylene-, wherein each R is independently hydrogen or optionally substituted C₁₋₆ aliphatic.

In certain embodiments, L is —NHC(O)CH═CH—, —NHC(O)CH═CHCH₂N(CH₃)—, —NHC(O)CH═CHCH₂O—, —CH₂NHC(O)CH═CH—, —NHSO₂CH═CH—, —NHSO₂CH═CHCH₂—, —NHC(O)(C═N₂)C(O)—, —NHC(O)CH═CHCH₂N(CH₃)—, —NHSO₂CH═CH—, —NHSO₂CH═CHCH₂—, —NHC(O)CH═CHCH₂O—, —NHC(O)C(═CH₂)CH₂—, —CH₂NHC(O)—, —CH₂NHC(O)CH═CH—, —CH₂CH₂NHC(O)—, or —CH₂NHC(O)cyclopropylene-.

In some embodiments, L is a bivalent C₂₋₈ straight or branched, hydrocarbon chain wherein L has at least one triple bond. In certain embodiments, L is a bivalent C₂₋₈ straight or branched, hydrocarbon chain wherein L has at least one triple bond and one or two additional methylene units of L are optionally and independently replaced by —NRC(O)—, —C(O)NR—, —S—, —S(O)—, —SO₂—, —C(═S)—, —C(═NR)—, —O—, —N(R)—, or —C(O)—. In some embodiments, L has at least one triple bond and at least one methylene unit of L is replaced by —N(R)—, —N(R)C(O)—, —C(O)—, —C(O)O—, or —OC(O)—, or —O—.

Exemplary L groups include —C≡C—, —C≡CCH₂N(isopropyl)-, —NHC(O)C≡CCH₂CH₂—, —CH₂—C≡C—CH₂—, —C≡CCH₂O—, —CH₂C(O)C≡C—, —C(O)C≡C—, or —CH₂OC(═O)C≡C—.

In certain embodiments, L is a bivalent C₂₋₈ straight or branched, hydrocarbon chain wherein one methylene unit of L is replaced by cyclopropylene and one or two additional methylene units of L are independently replaced by —C(O)—, —NRC(O)—, —C(O)NR—, —N(R)SO₂—, or —SO₂N(R)—. Exemplary L groups include —NHC(O)-cyclopropylene-SO₂— and —NHC(O)-cyclopropylene-.

As defined generally above, Y is hydrogen, C₁₋₆ aliphatic optionally substituted with oxo, halogen, NO₂, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with at 1-4 R^{e} groups, each R^{e} is independently selected from -Q-Z, oxo, NO₂, halogen, CN, a suitable leaving group, or C₁₋₆ aliphatic, wherein Q is a covalent bond or a bivalent C₁₋₆ saturated or unsaturated, straight or branched, hydrocarbon chain, wherein one or two methylene units of Q are optionally and independently replaced by —N(R)—S—, —O—, —C(O)—, —OC(O)—, —C(O)O—, —SO—, or —SO₂—, —N(R)C(O)—, —C(O)N(R)—, —N(R)SO₂—, or —SO₂N(R)—; and, Z is hydrogen or C₁₋₆ aliphatic optionally substituted with oxo, halogen, NO₂, or CN.

In certain embodiments, Y is hydrogen.

In certain embodiments, Y is C₁₋₆ aliphatic optionally substituted with oxo, halogen, NO₂, or CN. In some embodiments, Y is C₂₋₆ alkenyl optionally substituted with oxo, halogen, NO₂, or CN. In other embodiments, Y is C₂₋₆ alkynyl optionally substituted with oxo, halogen, NO₂, or CN. In some embodiments, Y is C₂₋₆ alkenyl. In other embodiments, Y is C₂₋₄ alkynyl.

In other embodiments, Y is C₁₋₆ alkyl substituted with oxo, halogen, NO₂, or CN. Such Y groups include —CH₂F, —CH₂Cl, —CH₂CN, and —CH₂NO₂.

In certain embodiments, Y is a saturated 3-6 membered monocyclic ring having 0-3 heteroatoms independently selected from nitrogen, oxygen, or sulfur, wherein Y is substituted with 1-4 R^{e} groups, wherein each R^{e} is as defined above and described herein.

In some embodiments, Y is a saturated 3-4 membered heterocyclic ring having 1 heteroatom selected from oxygen or nitrogen wherein said ring is substituted with 1-2 R^{e} groups, wherein each R^{e} is as defined above and described herein. Exemplary such rings are epoxide and oxetane rings, wherein each ring is substituted with 1-2 R^{e} groups, wherein each R^{e} is as defined above and described herein.

In other embodiments, Y is a saturated 5-6 membered heterocyclic ring having 1-2 heteroatom selected from oxygen or nitrogen wherein said ring is substituted with 1-4 R^{e} groups, wherein each R^{e} is as defined above and described herein. Such rings include piperidine and pyrrolidine, wherein each ring is substituted with 1-4 R^{e} groups, wherein each R^{e} is as defined above and described herein. In certain embodiments, Y is

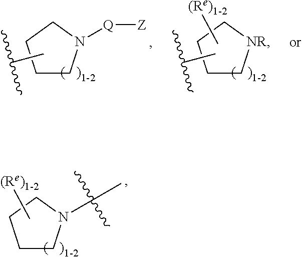

wherein each R, Q, Z, and R^{e} is defined above and described herein. In certain embodiments, Y is piperazine.

In some embodiments, Y is a saturated 3-6 membered carbocyclic ring, wherein said ring is substituted with 1-4 R^{e} groups, wherein each R^{e} is as defined above and described herein. In certain embodiments, Y is cyclopropyl, cyclobutyl, cyclopentyl, or cyclohexyl, wherein each ring is substituted with 1-4 R^{e} groups, wherein each R^{e} is as defined above and described herein. In certain embodiments, Y is

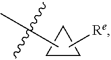

wherein R^{e} is as defined above and described herein. In certain embodiments, Y is cyclopropyl optionally substituted with halogen, CN or NO₂.

In certain embodiments, Y is a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein.

In some embodiments, Y is a partially unsaturated 3-6 membered carbocyclic ring, wherein said ring is substituted with 1-4 R^{e} groups, wherein each R^{e} is as defined above and described herein. In some embodiments, Y is cyclopropenyl, cyclobutenyl, cyclopentenyl, or cyclohexenyl wherein each ring is substituted with 1-4 R^{e} groups, wherein each R^{e} is as defined above and described herein. In certain embodiments, Y is

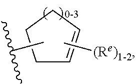

wherein each R^{e} is as defined above and described herein.

In certain embodiments, Y is a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein. In certain embodiments, Y is selected from:

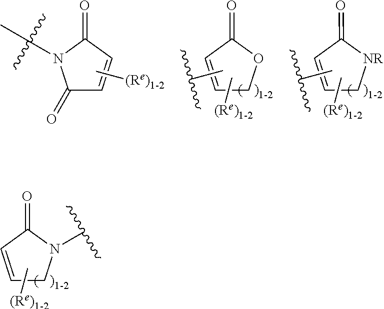

wherein each R and R^{e} is as defined above and described herein.

In certain embodiments, Y is a 6-membered aromatic ring having 0-2 nitrogens wherein said ring is substituted with 1-4 R^{e} groups, wherein each R^{e} group is as defined above and described herein. In certain embodiments, Y is phenyl, pyridyl, or pyrimidinyl, wherein each ring is substituted with 1-4 R^{e} groups, wherein each R^{e} is as defined above and described herein.

In some embodiments, Y is selected from:

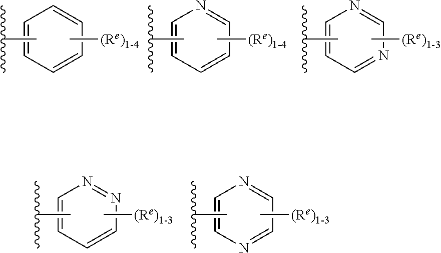

wherein each R^{e} is as defined above and described herein.

In other embodiments, Y is a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein. In some embodiments, Y is a 5 membered partially unsaturated or aryl ring having 1-3 heteroatoms independently selected from nitrogen, oxygen, and sulfur, wherein said ring is substituted with 1-4 R^{e} groups, wherein each R^{e} group is as defined above and described herein. Exemplary such rings are isoxazolyl, oxazolyl, thiazolyl, imidazolyl, pyrazolyl, pyrrolyl, furanyl, thienyl, triazole, thiadiazole, and oxadiazole, wherein each ring is substituted with 1-3 R^{e} groups, wherein each R^{e} group is as defined above and described herein. In certain embodiments, Y is selected from:

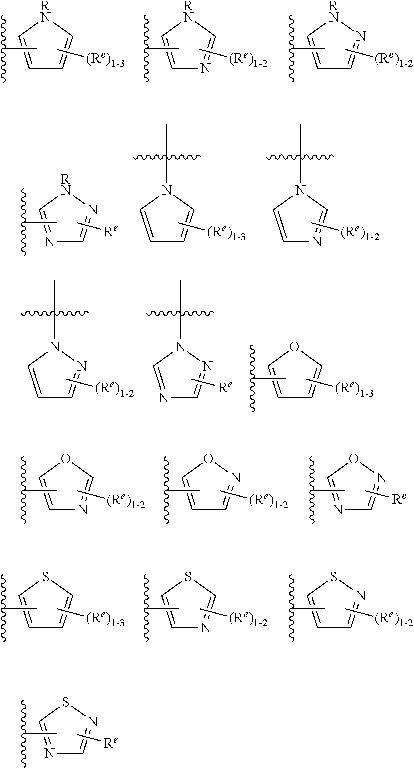

wherein each R and R^{e} is as defined above and described herein.

In certain embodiments, Y is an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein. According to another aspect, Y is a 9-10 membered bicyclic, partially unsaturated, or aryl ring having 1-3 heteroatoms independently selected from nitrogen, oxygen, or sulfur, wherein said ring is substituted with 1-4 R^{e} groups, wherein R^{e} is as defined above and described herein. Exemplary such bicyclic rings include 2,3-dihydrobenzo[d]isothiazole, wherein said ring is substituted with 1-4 R^{e} groups, wherein R^{e} is as defined above and described herein.

As defined generally above, each R^{e} group is independently selected from -Q-Z, oxo, NO₂, halogen, CN, a suitable leaving group, or C₁₋₆ aliphatic optionally substituted with oxo, halogen, NO₂, or CN, wherein Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, or —SO₂—, —N(R)C(O)—, —C(O)N(R)—, —N(R)SO₂—, or —SO₂N(R)—; and Z is hydrogen or C₁₋₆ aliphatic optionally substituted with oxo, halogen, NO₂, or CN.

In certain embodiments, R^{e} is C₁₋₆ aliphatic optionally substituted with oxo, halogen, NO₂, or CN. In other embodiments, R^{e} is oxo, NO₂, halogen, or CN.

In some embodiments, R^{e} is -Q-Z, wherein Q is a covalent bond and Z is hydrogen (i.e., R^{e} is hydrogen). In other embodiments, R^{e} is -Q-Z, wherein Q is a bivalent C₁₋₆ saturated or unsaturated, straight or branched, hydrocarbon chain, wherein one or two methylene units of Q are optionally and independently replaced by —NR—, —NRC(O)—, —C(O)NR—, —S—, —O—, —C(O)—, —SO—, or —SO₂—. In other embodiments, Q is a bivalent C₂₋₆ straight or branched, hydrocarbon chain having at least one double bond, wherein one or two methylene units of Q are optionally and independently replaced by —NR—, —NRC(O)—, —C(O)NR—, —S—, —O—, —C(O)—, —SO—, or —SO₂—. In certain embodiments, the Z moiety of the R^{e} group is hydrogen. In some embodiments, -Q-Z is —NHC(O)CH═CH₂ or —C(O)CH═CH₂.

In certain embodiments, each R^{e} is independently selected from oxo, NO₂, CN, fluoro, chloro, —NHC(O)CH═CH₂, —C(O)CH═CH₂, —CH₂CH═CH₂, —C≡CH, —C(O)OCH₂Cl, —C(O)OCH₂F, —C(O)OCH₂CN, —C(O)CH₂Cl, —C(O)CH₂F, —C(O)CH₂CN, or —CH₂C(O)CH₃.

In certain embodiments, R^{e} is a suitable leaving group, ie a group that is subject to nucleophilic displacement. A “suitable leaving” is a chemical group that is readily displaced by a desired incoming chemical moiety such as the thiol moiety of a cysteine of interest. Suitable leaving groups are well known in the art, e.g., see, “Advanced Organic Chemistry,” Jerry March, 5^{th} Ed., pp. 351-357, John Wiley and Sons, N.Y. Such leaving groups include, but are not limited to, halogen, alkoxy, sulphonyloxy, optionally substituted alkylsulphonyloxy, optionally substituted alkenylsulfonyloxy, optionally substituted arylsulfonyloxy, acyloxy, and diazonium moieties. Examples of suitable leaving groups include chloro, iodo, bromo, fluoro, acetoxy, methanesulfonyloxy (mesyloxy), tosyloxy, triflyloxy, nitro-phenylsulfonyloxy (nosyloxy), and bromo-phenylsulfonyloxy (brosyloxy).

In certain embodiments, the following embodiments and combinations of -L-Y apply:
(a) L is a bivalent C₂₋₈ straight or branched, hydrocarbon chain wherein L has at least one double bond and one or two additional methylene units of L are optionally and independently replaced by —NRC(O)—, —C(O)NR—, —N(R)SO₂—, —SO₂N(R)—, —S—, —S(O)—, —SO₂—, —OC(O)—, —C(O)O—, cyclopropylene, —O—, —N(R)—, or —C(O)—; and Y is hydrogen or C₁₋₆ aliphatic optionally substituted with oxo, halogen, NO₂, or CN; or
(b) L is a bivalent C₂₋₈ straight or branched, hydrocarbon chain wherein L has at least one double bond and at least one methylene unit of L is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L are optionally and independently replaced by cyclopropylene, —O—, —N(R)—, or —C(O)—; and Y is hydrogen or C₁₋₆ aliphatic optionally substituted with oxo, halogen, NO₂, or CN; or
(c) L is a bivalent C₂₋₈ straight or branched, hydrocarbon chain wherein L has at least one double bond and at least one methylene unit of L is replaced by —C(O)—, and one or two additional methylene units of L are optionally and independently replaced by cyclopropylene, —O—, —N(R)—, or —C(O)—; and Y is hydrogen or C₁₋₆ aliphatic optionally substituted with oxo, halogen, NO₂, or CN; or
(d) L is a bivalent C₂₋₈ straight or branched, hydrocarbon chain wherein L has at least one double bond and at least one methylene unit of L is replaced by —C(O)—; and Y is hydrogen or C₁₋₆ aliphatic optionally substituted with oxo, halogen, NO₂, or CN; or
(e) L is a bivalent C₂₋₈ straight or branched, hydrocarbon chain wherein L has at least one double bond and at least one methylene unit of L is replaced by —OC(O)—; and Y is hydrogen or C₁₋₆ aliphatic optionally substituted with oxo, halogen, NO₂, or CN; or
(f) L is —NRC(O)CH═CH—, —NRC(O)CH═CHCH₂N(CH₃)—, —NRC(O)CH═CHCH₂O—, —CH₂NRC(O)CH═CH—, —NRSO₂CH═CH—, —NRSO₂CH═CHCH₂—, —NRC(O)(C═N₂)—, —NRC(O)(C═N₂)C(O)—, —NRC(O)CH═CHCH₂N(CH₃)—, —NRSO₂CH═CH—, —NRSO₂CH═CHCH₂—, —NRC(O)CH═CHCH₂O—, —NRC(O)C(═CH₂)CH₂—, —CH₂NRC(O)—, —CH₂NRC(O)CH═CH—, —CH₂CH₂NRC(O)—, or —CH₂NRC(O)cyclopropylene-; wherein R is H or optionally substituted C₁₋₆ aliphatic; and Y is hydrogen or C₁₋₆ aliphatic optionally substituted with oxo, halogen, NO₂, or CN; or
(g) L is —NHC(O)CH═CH—, —NHC(O)CH═CHCH₂N(CH₃)—, —NHC(O)CH═CHCH₂O—, —CH₂NHC(O)CH═CH—, —NHSO₂CH═CH—, —NHSO₂CH═CHCH₂—, —NHC(O)(C═N₂)—, —NHC(O)(C═N₂)C(O)—, —NHC(O)CH═CHCH₂N(CH₃)—, —NHSO₂CH═CH—, —NHSO₂CH═CHCH₂—, —NHC(O)CH═CHCH₂O—, —NHC(O)C(═CH₂)CH₂—, —CH₂NHC(O)—, —CH₂NHC(O)CH═CH—, —CH₂CH₂NHC(O)—, or —CH₂NHC(O)cyclopropylene-; and Y is hydrogen or C₁₋₆ aliphatic optionally substituted with oxo, halogen, NO₂, or CN; or
(h) L is a bivalent C₂₋₈ straight or branched, hydrocarbon chain wherein L has at least one alkylidenyl double bond and at least one methylene unit of L is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L are optionally and independently replaced by cyclopropylene, —O—, —N(R)—, or —C(O)—; and Y is hydrogen or C₁₋₆ aliphatic optionally substituted with oxo, halogen, NO₂, or CN; or
(i) L is a bivalent C₂₋₈ straight or branched, hydrocarbon chain wherein L has at least one triple bond and one or two additional methylene units of L are optionally and independently replaced by —NRC(O)—, —C(O)NR—, —N(R)SO₂—, —SO₂N(R)—, —S—, —S(O)—, —SO₂—, —OC(O)—, or —C(O)O—, and Y is hydrogen or C₁₋₆ aliphatic optionally substituted with oxo, halogen, NO₂, or CN; or
(j) L is —C≡C—, —C≡CCH₂N(isopropyl)-, —NHC(O)C≡CCH₂CH₂—, —CH₂—C≡C—CH₂—, —C≡CCH₂O—, —CH₂C(O)C≡C—, —C(O)C≡C—, or —CH₂C(═O)C≡C—; and Y is hydrogen or C₁₋₆ aliphatic optionally substituted with oxo, halogen, NO₂, or CN; or
(k) L is a bivalent C₂₋₈ straight or branched, hydrocarbon chain wherein one methylene unit of L is replaced by cyclopropylene and one or two additional methylene units of L are independently replaced by —NRC(O)—, —C(O)NR—, —N(R)SO₂—, —SO₂N(R)—, —S—, —S(O)—, —SO₂—, —OC(O)—, or —C(O)O—; and Y is hydrogen or C₁₋₆ aliphatic optionally substituted with oxo, halogen, NO₂, or CN; or
(l) L is a covalent bond and Y is selected from:
   (i) C₁₋₆ alkyl substituted with oxo, halogen, NO₂, or CN;
   (ii) C₂₋₆ alkenyl optionally substituted with oxo, halogen, NO₂, or CN; or
   (iii) C₂₋₆ alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or
   (vi)

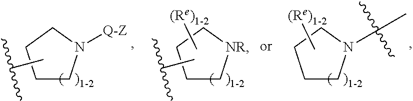

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or
(x)

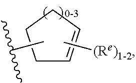

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or
(xii)

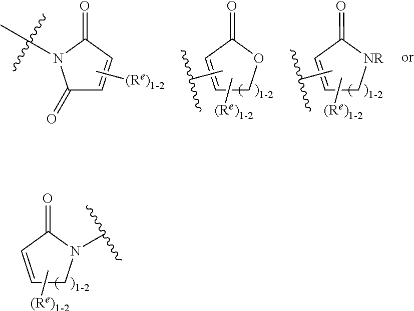

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or
(xiv)

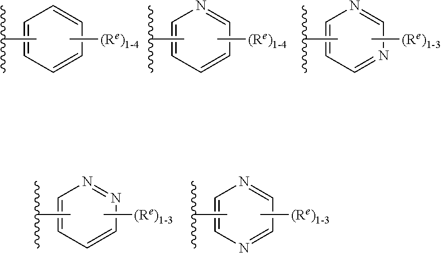

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or
(xvi)

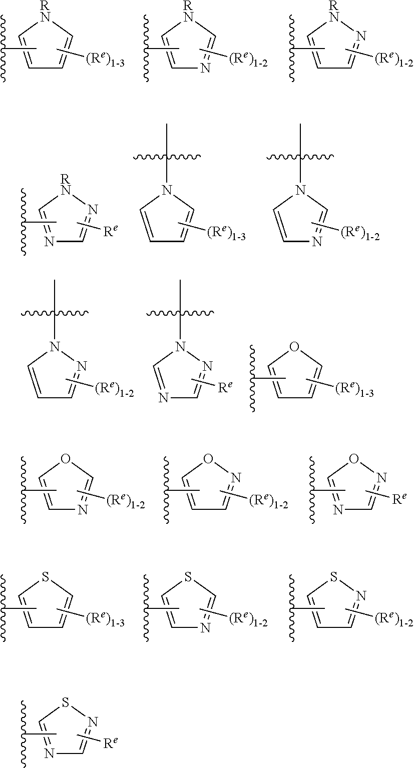

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(m) L is —C(O)— and Y is selected from:
   (i) C₁₋₆ alkyl substituted with oxo, halogen, NO₂, or CN; or
   (ii) C₂₋₆ alkenyl optionally substituted with oxo, halogen, NO₂, or CN; or
   (iii) C₂₋₆ alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or
   (vi)

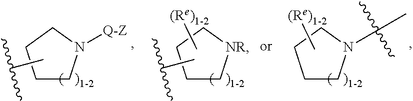

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or
(x)

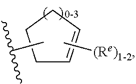

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or
(xii)

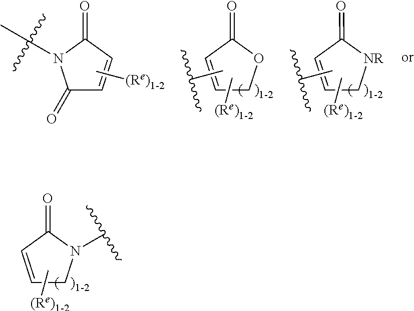

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or
(xiv)

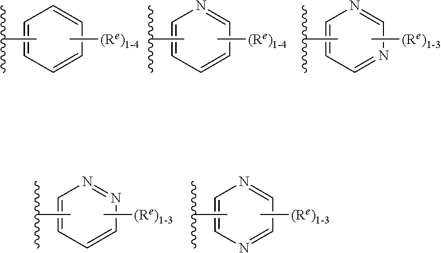

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or
(xvi)

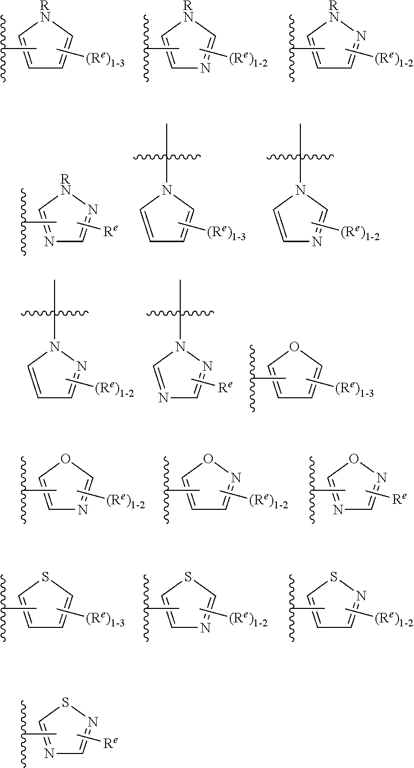

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(n) L is —N(R)C(O)— and Y is selected from:
   (i) C₁₋₆ alkyl substituted with oxo, halogen, NO₂, or CN; or
   (ii) C₂₋₆ alkenyl optionally substituted with oxo, halogen, NO₂, or CN; or
   (iii) C₂₋₆ alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or
   (vi)

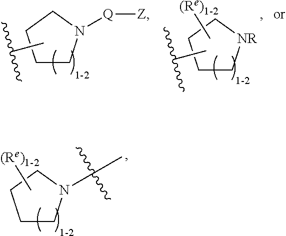

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or
(x)

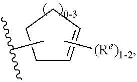

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or
(xii)

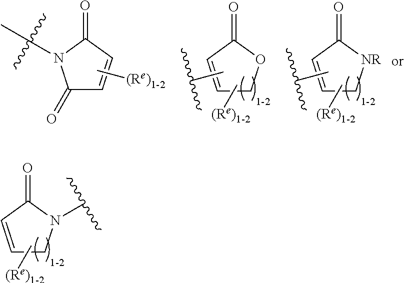

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or
(xiv)

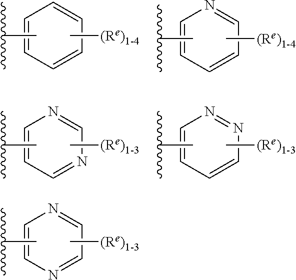

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or
(xvi)

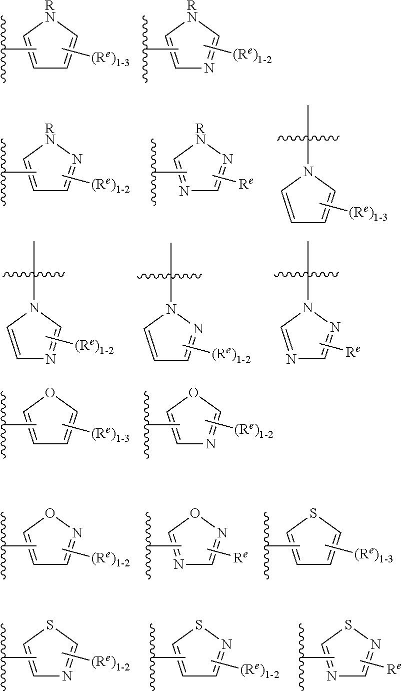

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(o) L is a bivalent C₁₋₈ saturated or unsaturated, straight or branched, hydrocarbon chain; and Y is selected from:
   (i) C₁₋₆ alkyl substituted with oxo, halogen, NO₂, or CN;
   (ii) C₂₋₆ alkenyl optionally substituted with oxo, halogen, NO₂, or CN; or
   (iii) C₂₋₆ alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or
   (vi)

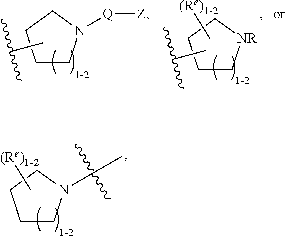

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or
(x)

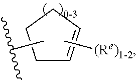

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or
(xii)

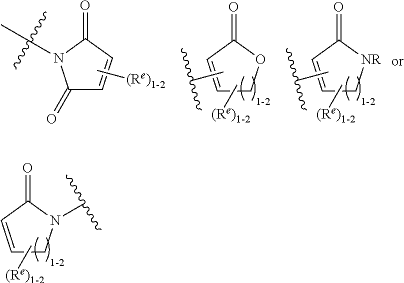

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or
(xiv)

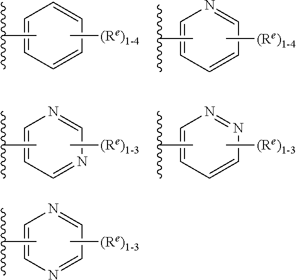

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or
(xvi)

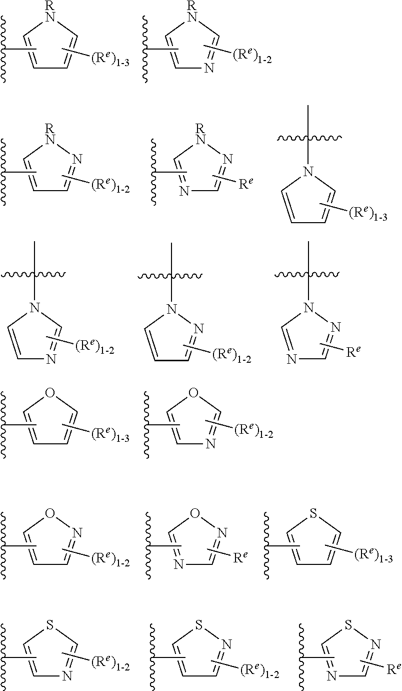

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(p) L is a covalent bond, —CH₂—, —NH—, —C(O)—, —CH₂NH—, —NHCH₂—, —NHC(O)—, —NHC(O)CH₂OC(O)—, —CH₂NHC(O)—, —NHSO₂—, —NHSO₂CH₂—, —NHC(O)CH₂OC(O)—, or —SO₂NH—; and Y is selected from:
   (i) C₁₋₆ alkyl substituted with oxo, halogen, NO₂, or CN; or
   (ii) C₂₋₆ alkenyl optionally substituted with oxo, halogen, NO₂, or CN; or
   (iii) C₂₋₆ alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or
   (vi)

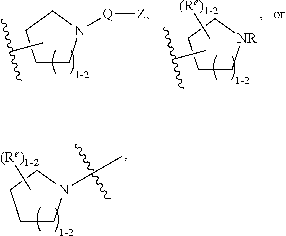

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or
(x)

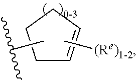

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or
(xii)

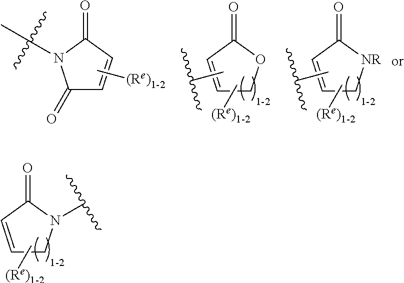

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or
(xiv)

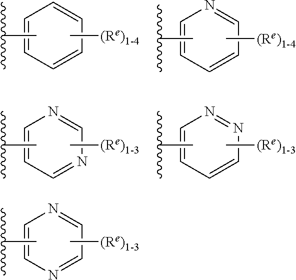

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or
(xvi)

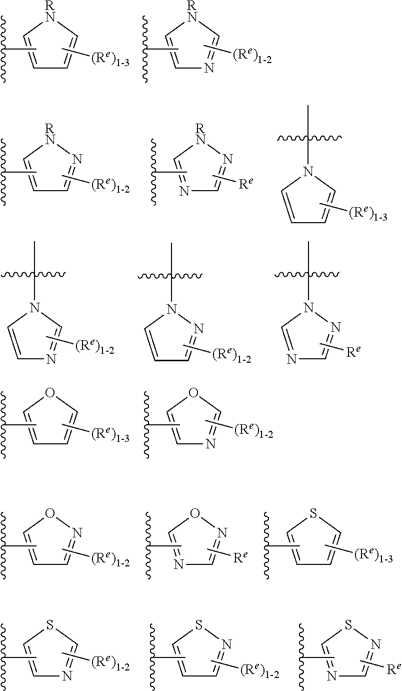

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein.

(q) L is a bivalent C₂₋₈ straight or branched, hydrocarbon chain wherein two or three methylene units of L are optionally and independently replaced by —NRC(O)—, —C(O)NR—, —N(R)SO₂—, —SO₂N(R)—, —S—, —S(O)—, —SO₂—, —OC(O)—, —C(O)O—, cyclopropylene, —O—, —N(R)—, or —C(O)—; and Y is hydrogen or C₁₋₆ aliphatic optionally substituted with oxo, halogen, NO₂, or CN.

In certain embodiments, the Y group of formula I is selected from those set forth in Table 3, below, wherein each wavy line indicates the point of attachment to the rest of the molecule.

TABLE 3
Exemplary Y groups:

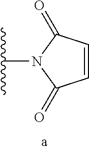

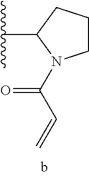

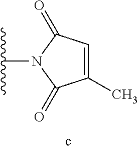

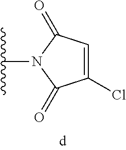

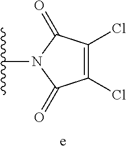

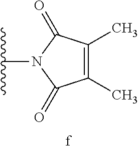

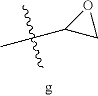

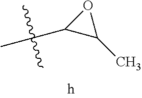

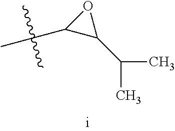

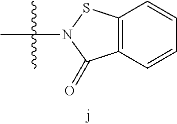

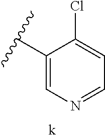

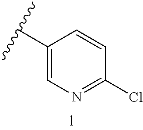

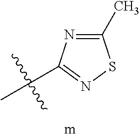

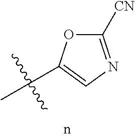

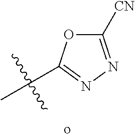

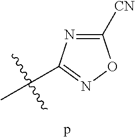

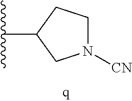

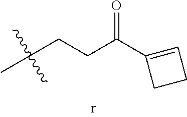

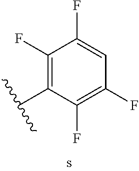

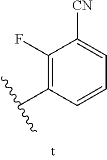

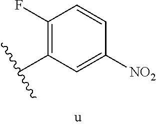

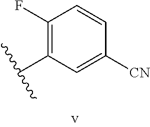

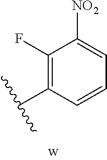

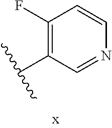

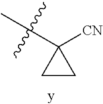

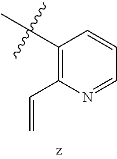

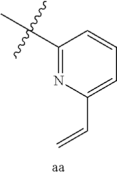

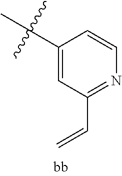

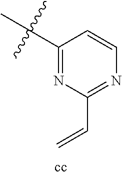

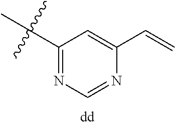

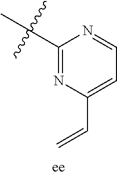

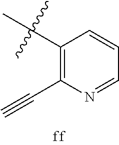

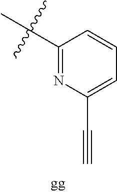

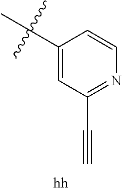

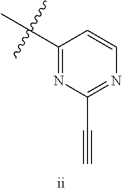

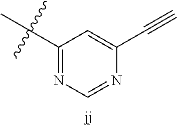

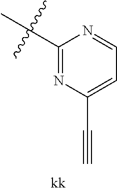

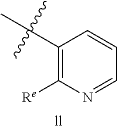

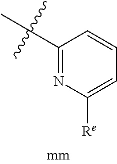

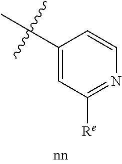

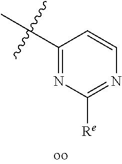

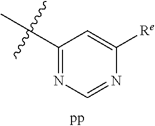

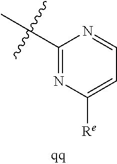

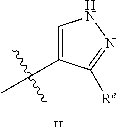

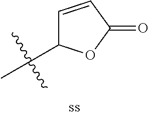

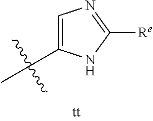

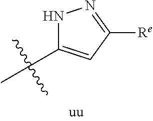

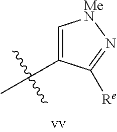

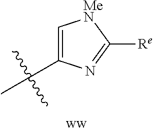

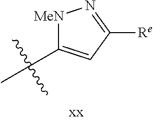

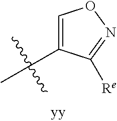

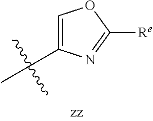

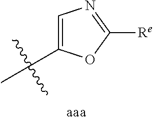

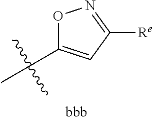

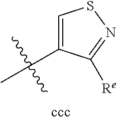

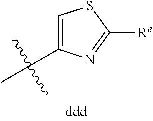

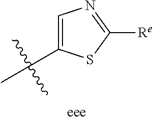

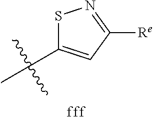

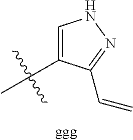

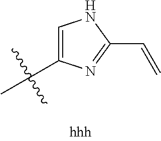

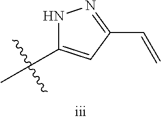

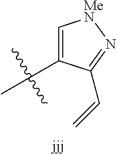

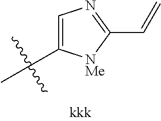

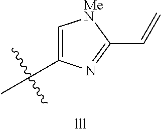

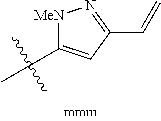

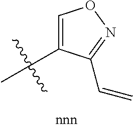

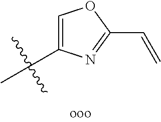

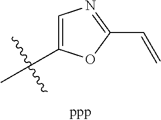

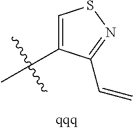

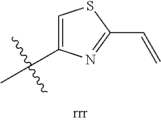

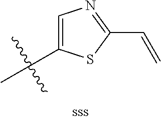

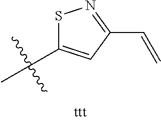

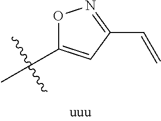

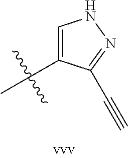

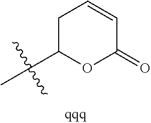

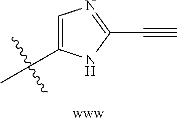

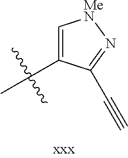

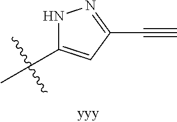

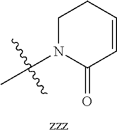

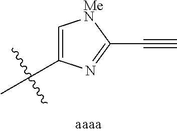

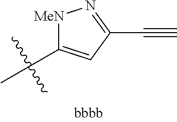

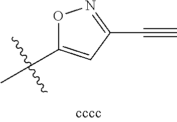

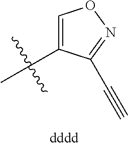

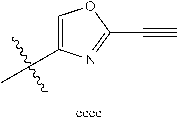

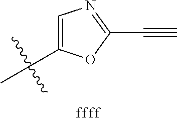

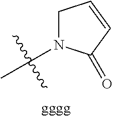

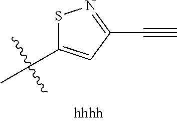

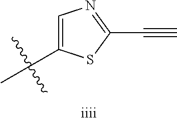

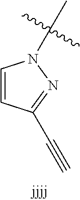

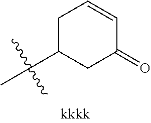

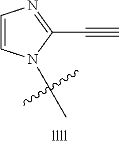

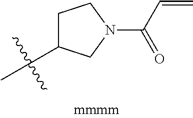

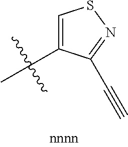

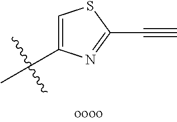

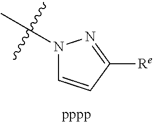

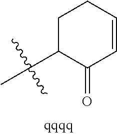

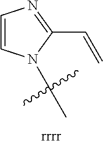

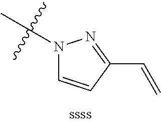

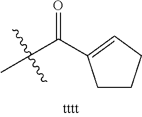

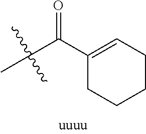

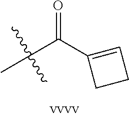

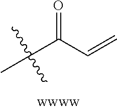

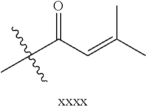

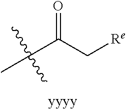

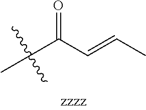

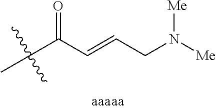

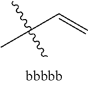

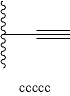

wherein each R^{e} is independently a suitable leaving group, NO₂, CN, or oxo.

In certain embodiments, R¹ is —C≡CH, —C≡CCH₂NH(isopropyl), —NHC(O)C≡CCH₂CH₃, —CH₂—C≡C—CH₃, —C≡CCH₂OH, —CH₂C(O)C≡CH, —C(O)C≡CH, or —CH₂C(═O)C≡CH. In some embodiments, R¹ is selected from —NHC(O)CH═CH₂, —NHC(O)CH═CHCH₂N(CH₃)₂, or —CH₂NHC(O)CH═CH₂.

In some embodiments, R¹ is 6-12 atoms long. In certain embodiments, R¹ is 6-9 atoms long. In certain embodiments, R¹ is 10-12 atoms long. In certain embodiments, R¹ is at least 8 atoms long.

In certain embodiments, R¹ is —C(O)CH₂CH₂C(O)CH═C(CH₃)₂, —C(O)CH₂CH₂C(O)CH═CH(cyclopropyl), —C(O)CH₂CH₂C(O)CH═CHCH₃, —C(O)CH₂CH₂C(O)CH═CHCH₂CH₃, or —C(O)CH₂CH₂C(O)C(═CH₂)CH₃. In certain embodiments, R¹ is —C(O)CH₂NHC(O)CH═CH₂, —C(O)CH₂NHC(O)CH₂CH₂C(O)CH═CHCH₃, or —C(O)CH₂NHC(O)CH₂CH₂C(O)C(═CH₂)CH₃. In certain embodiments, R¹ is —S(O)₂CH₂CH₂NHC(O)CH₂CH₂C(O)CH═C(CH₃)₂, —S(O)₂CH₂CH₂NHC(O)CH₂CH₂C(O)CH═CHCH₃, or —S(O)₂CH₂CH₂NHC(O)CH₂CH₂C(O)CH═CH₂. In certain embodiments, R¹ is —C(O)(CH₂)₃NHC(O)CH₂CH₂C(O)CH═CHCH₃ or —C(O)(CH₂)₃NHC(O)CH₂CH₂C(O)CH═CH₂.

In certain embodiments, R¹ is selected from those set forth in Table 4, below, wherein each wavy line indicates the point of attachment to the rest of the molecule.

TABLE 4
Exemplary R¹ Groups

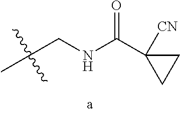

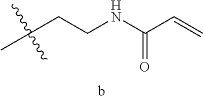

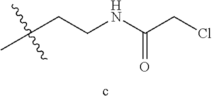

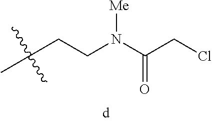

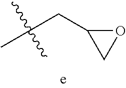

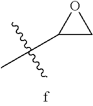

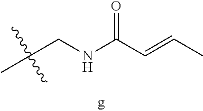

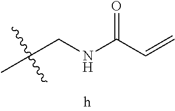

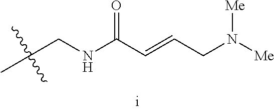

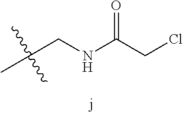

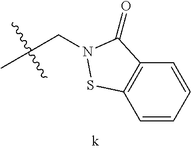

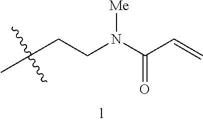

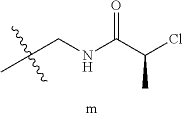

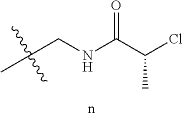

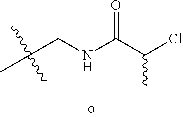

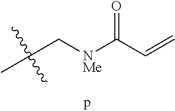

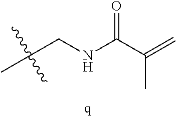

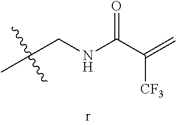

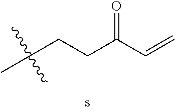

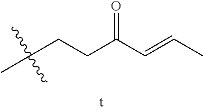

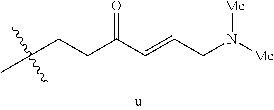

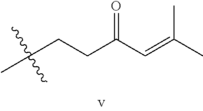

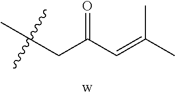

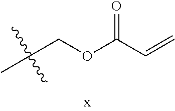

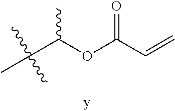

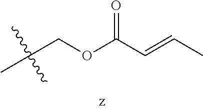

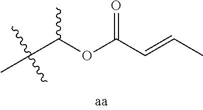

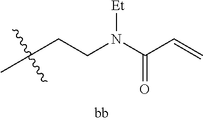

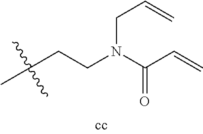

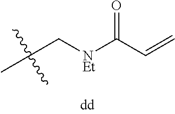

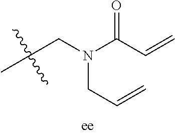

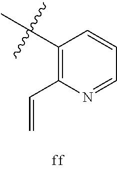

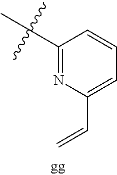

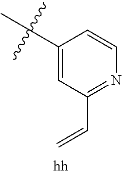

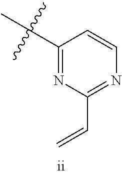

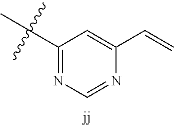

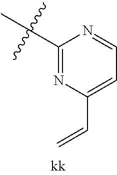

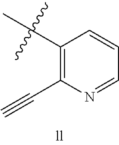

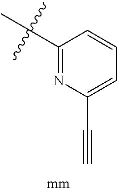

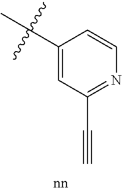

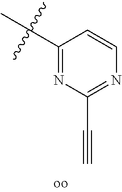

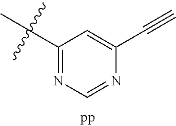

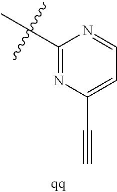

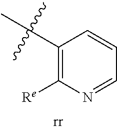

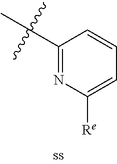

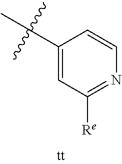

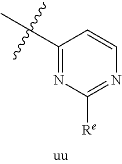

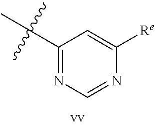

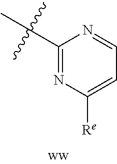

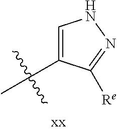

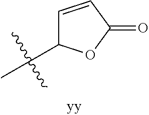

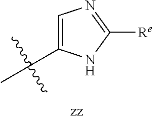

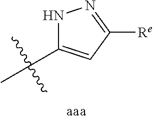

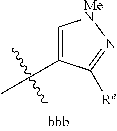

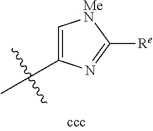

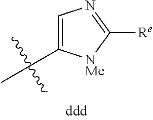

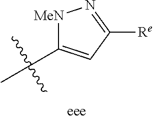

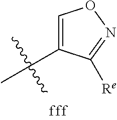

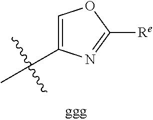

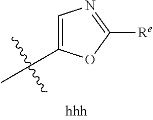

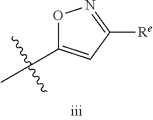

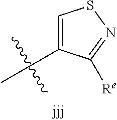

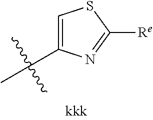

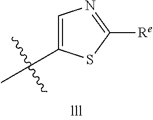

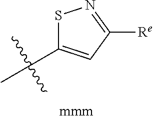

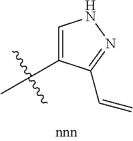

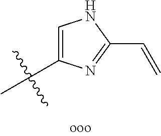

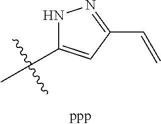

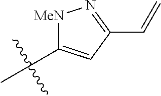

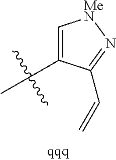

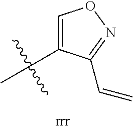

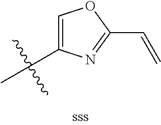

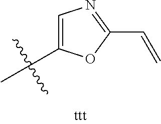

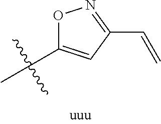

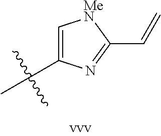

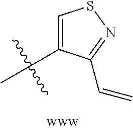

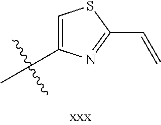

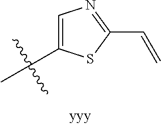

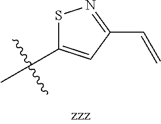

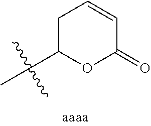

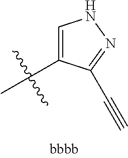

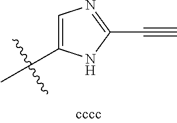

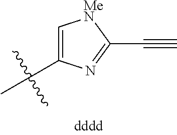

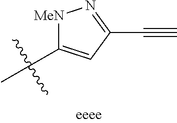

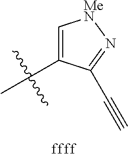

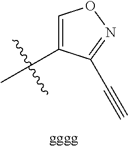

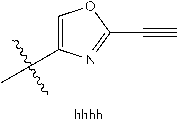

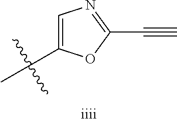

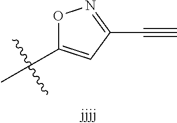

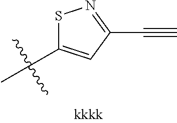

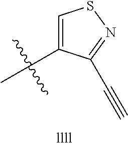

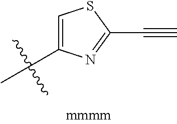

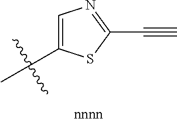

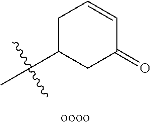

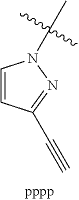

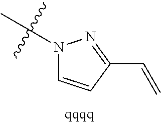

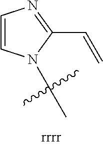

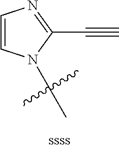

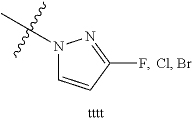

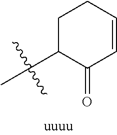

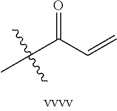

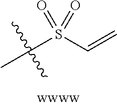

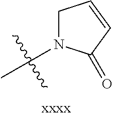

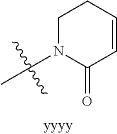

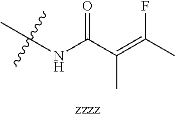

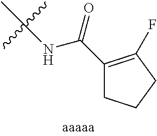

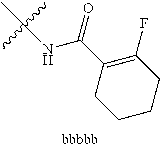

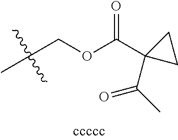

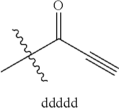

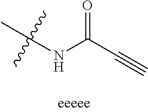

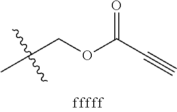

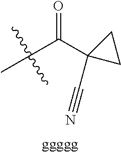

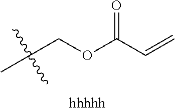

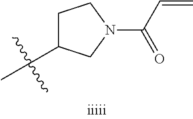

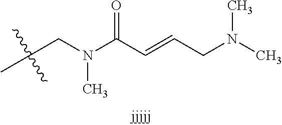

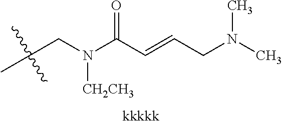

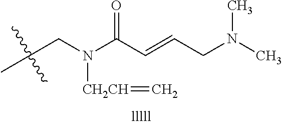

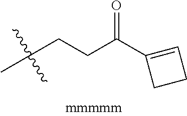

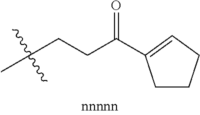

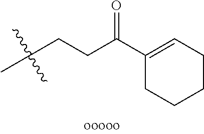

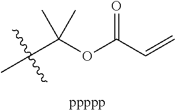

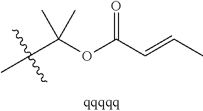

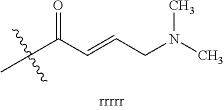

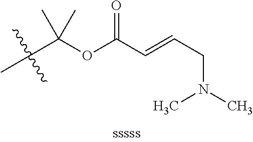

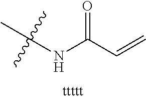

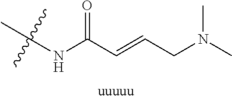

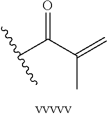

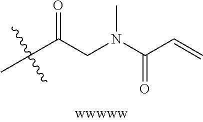

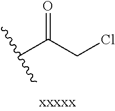

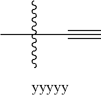

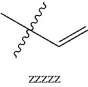

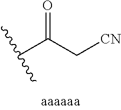

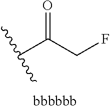

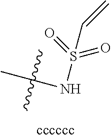

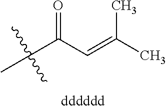

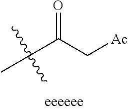

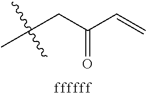

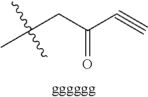

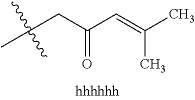

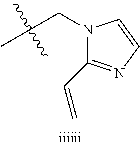

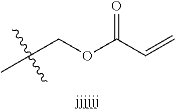

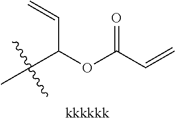

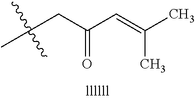

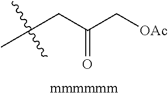

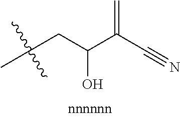

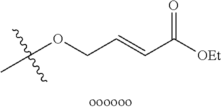

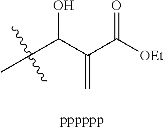

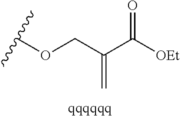

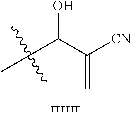

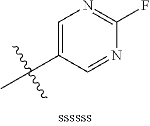

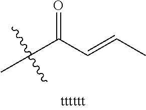

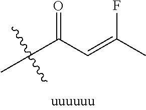

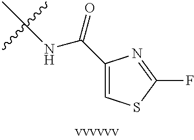

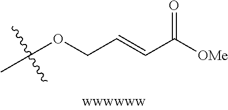

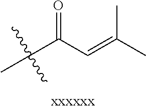

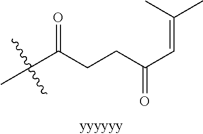

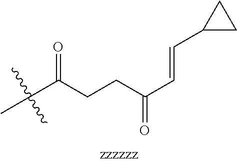

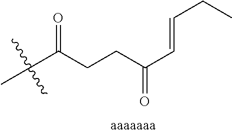

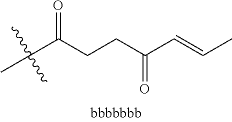

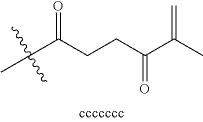

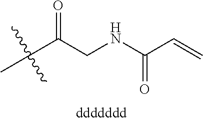

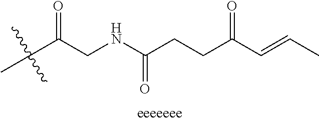

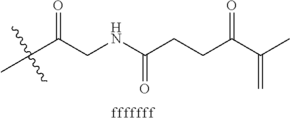

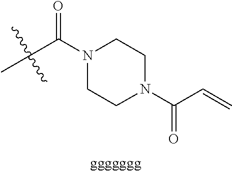

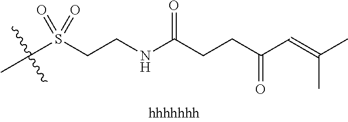

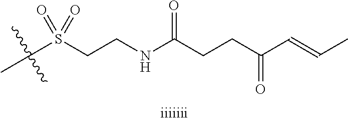

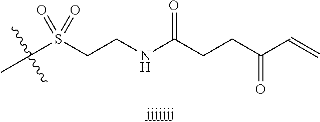

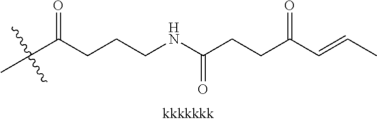

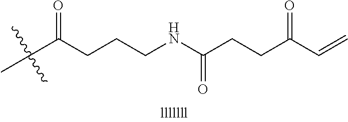

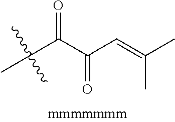

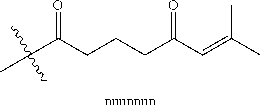

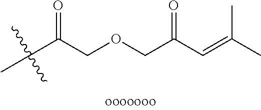

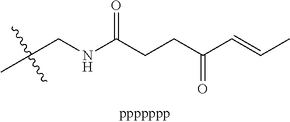

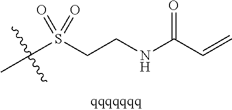

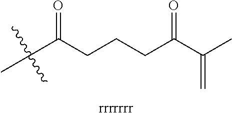

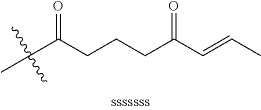

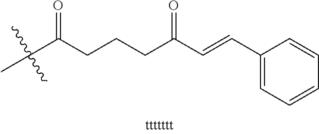

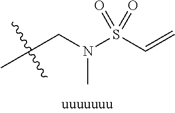

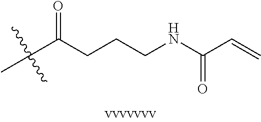

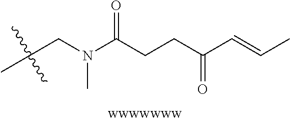

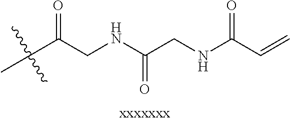

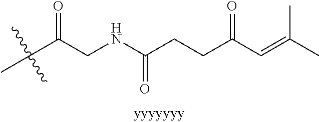

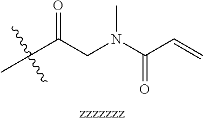

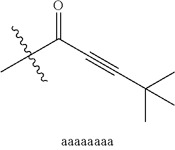

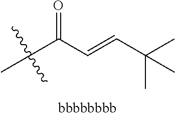

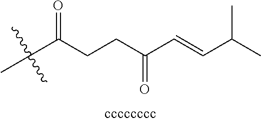

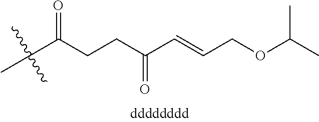

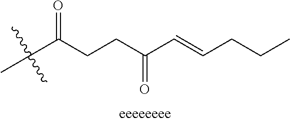

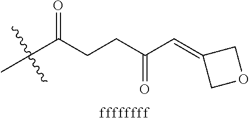

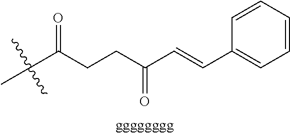

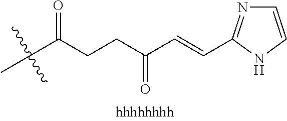

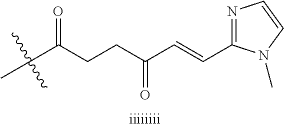

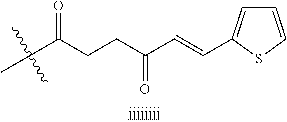

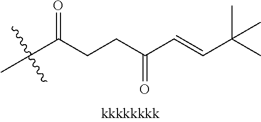

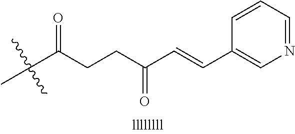

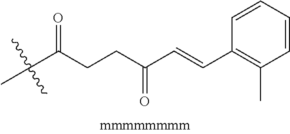

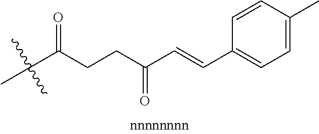

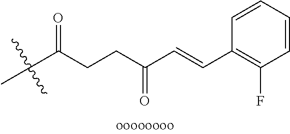

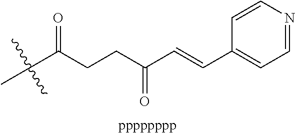

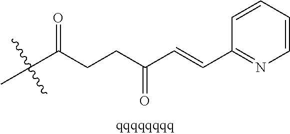

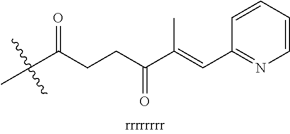

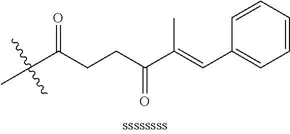

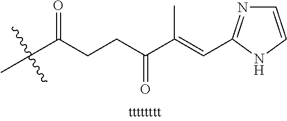

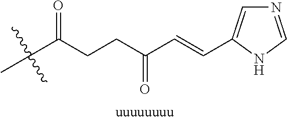

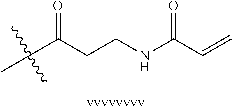

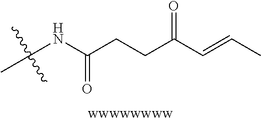

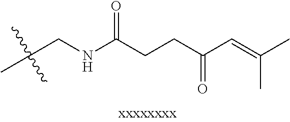

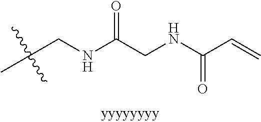

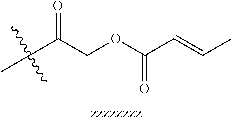

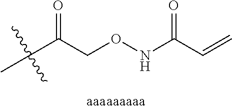

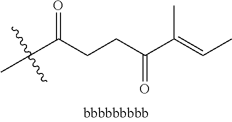

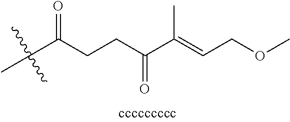

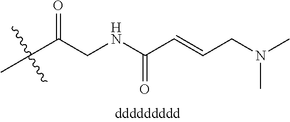

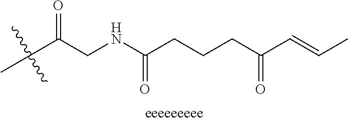

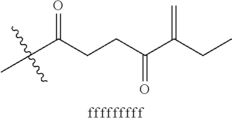

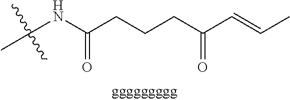

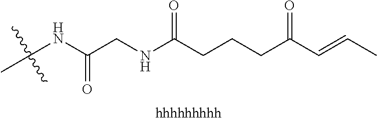

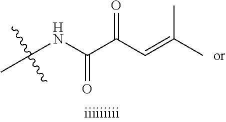

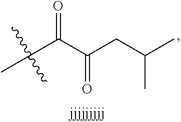

wherein each R^{e} is independently a suitable leaving group, NO₂, CN, or oxo.

In certain embodiments, R¹ is selected from:

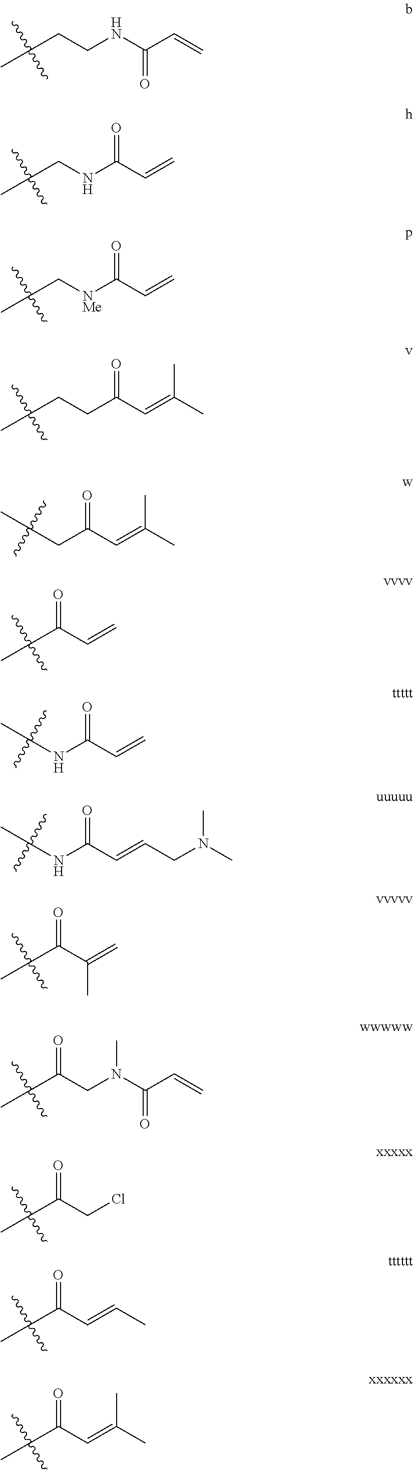

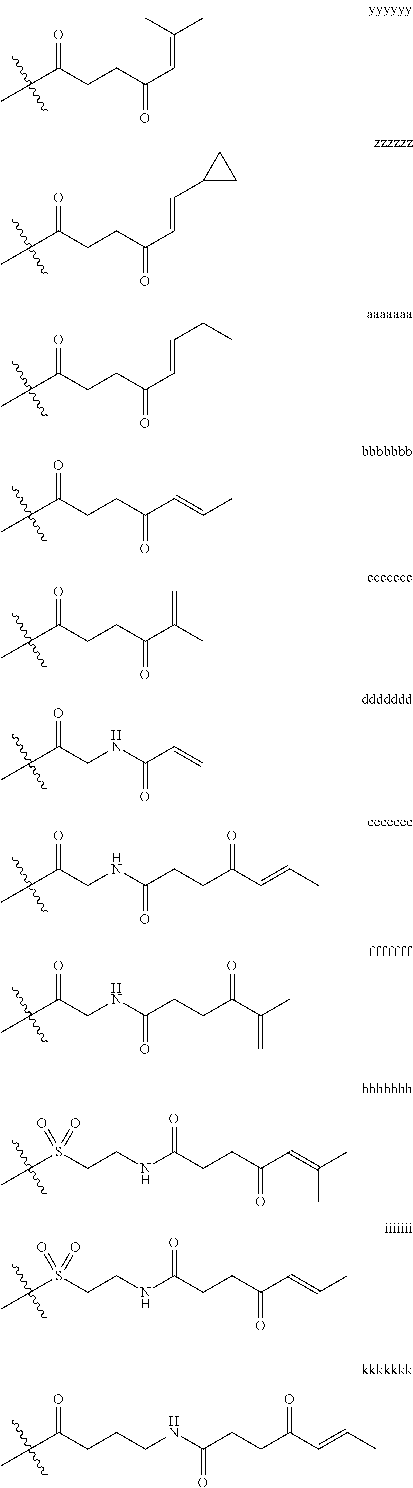

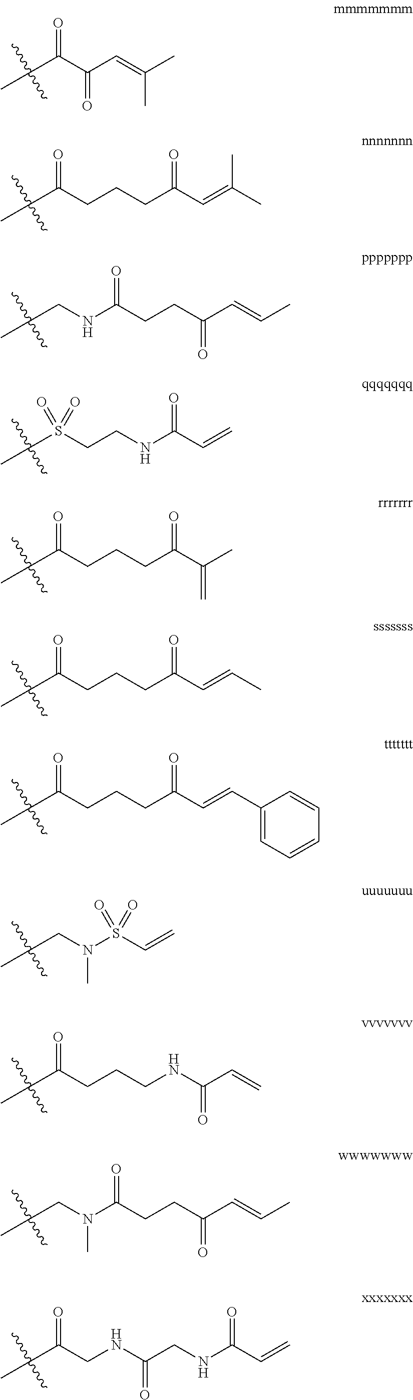

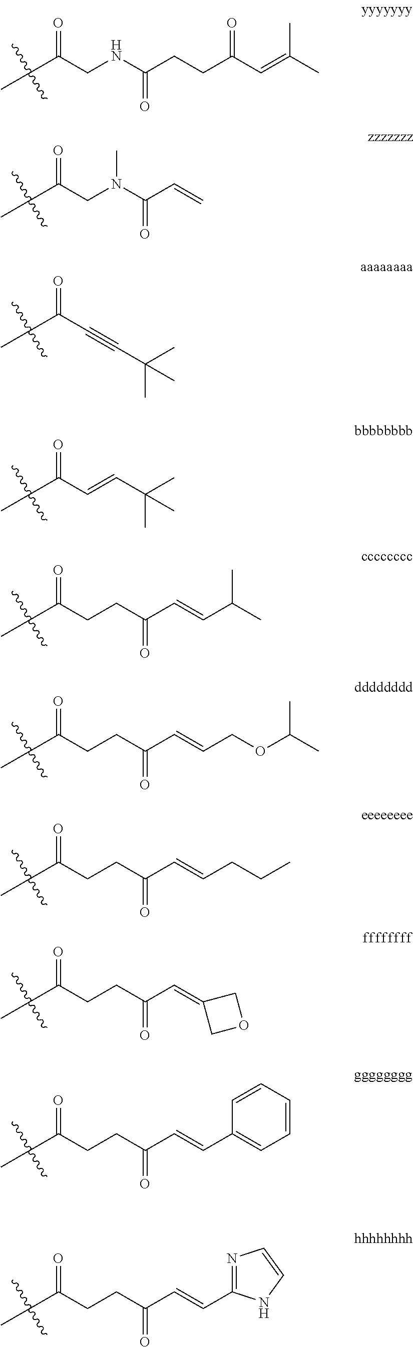

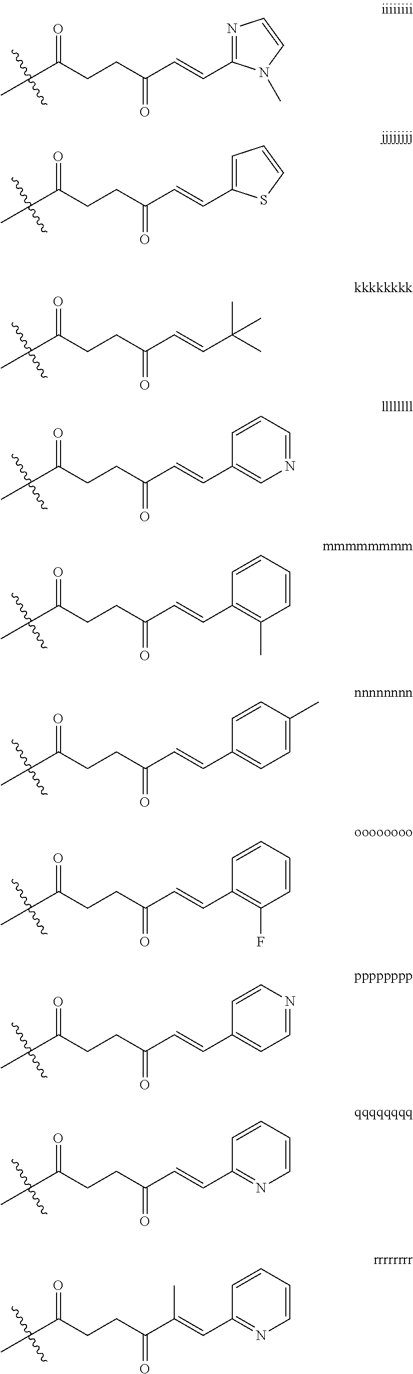

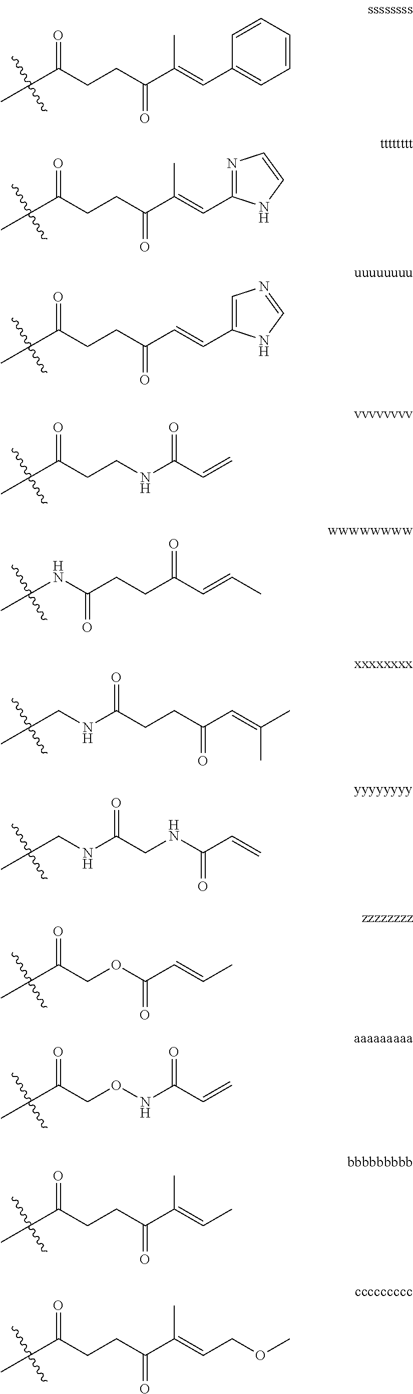

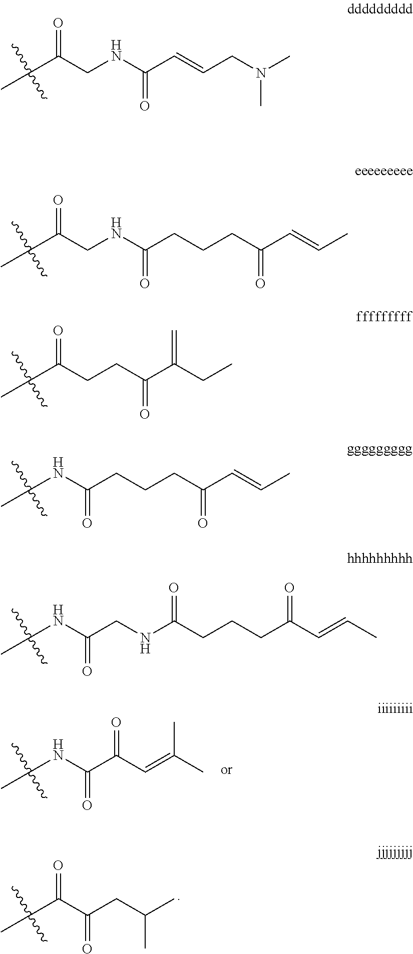

In certain embodiments, R¹ is selected from:

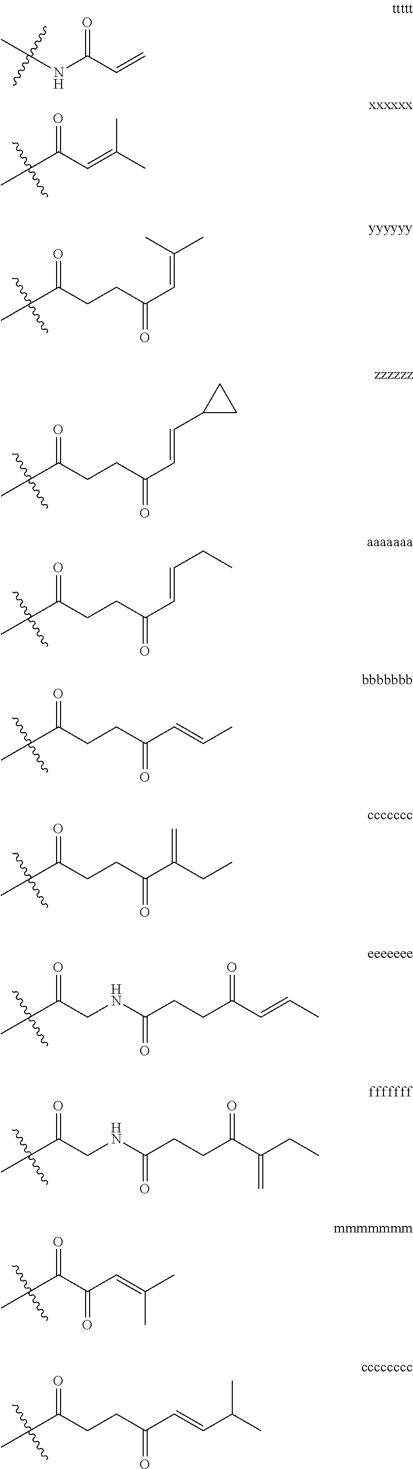

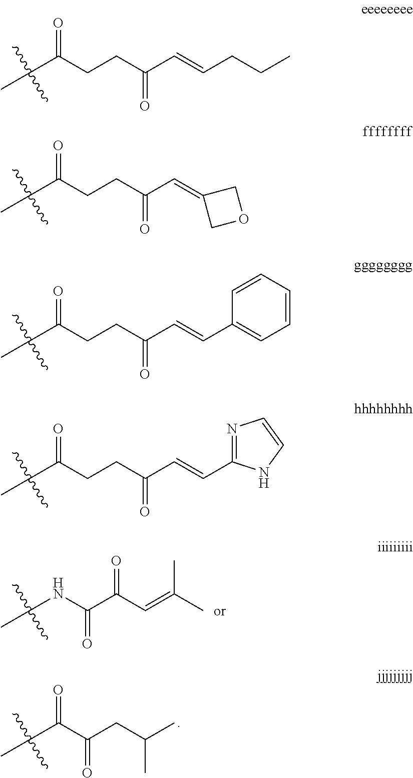

Exemplary compounds of formula I are set forth in Table 5, below:

TABLE 5
Exemplary Compounds of Formula I

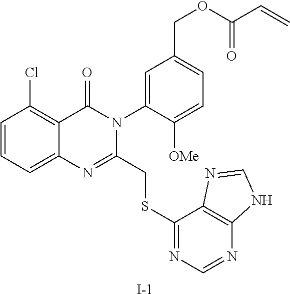

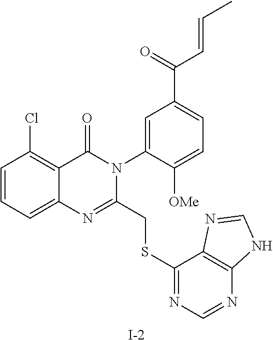

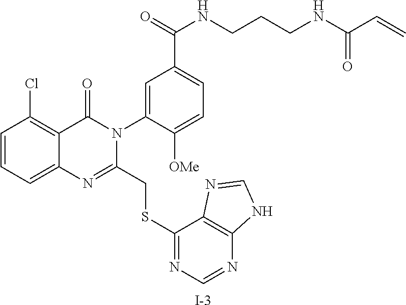

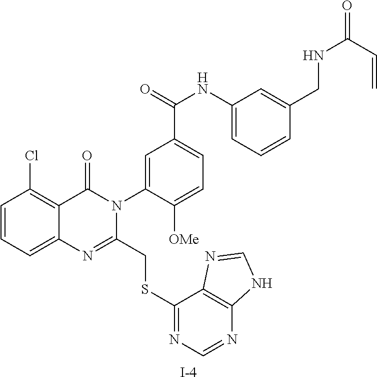

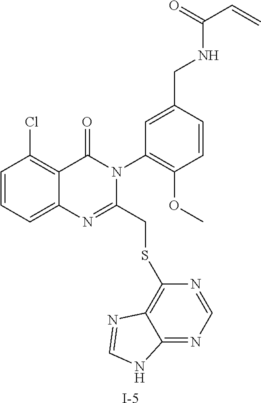

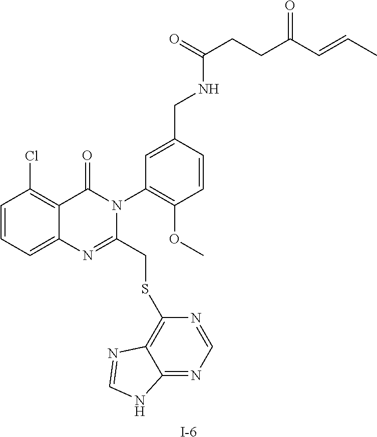

In certain embodiments, the present invention provides any compound selected from those depicted in Table 5, above, or a pharmaceutically acceptable salt thereof.

Exemplary compounds of formula II-a are set forth in Table 6, below:

TABLE 6
Exemplary Compounds of Formula II-a

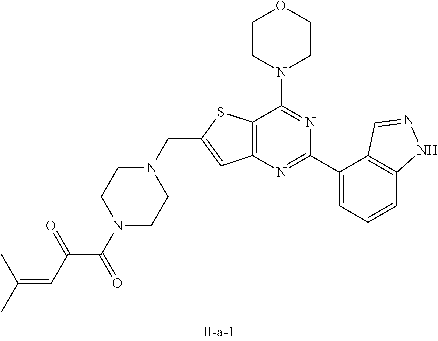

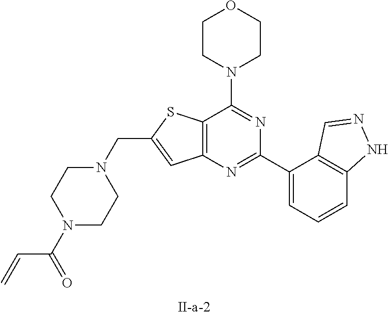

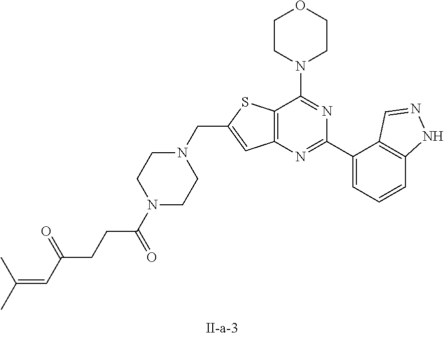

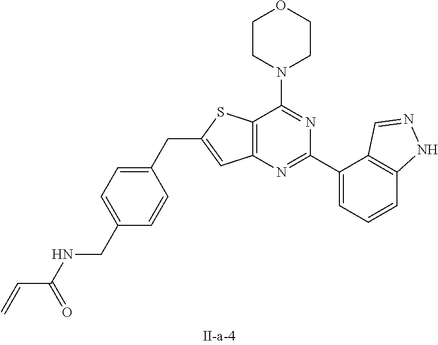

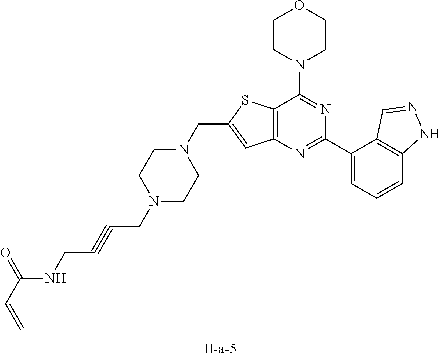

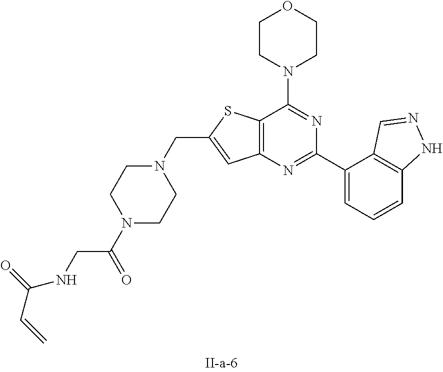

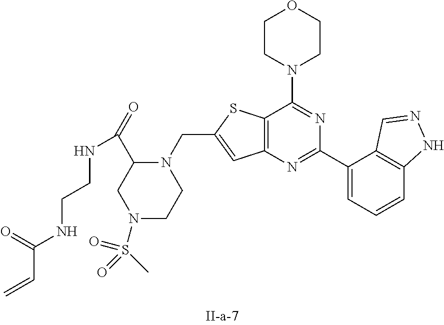

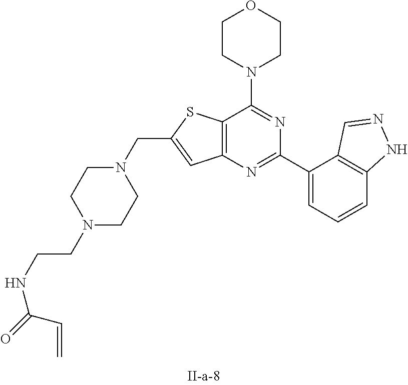

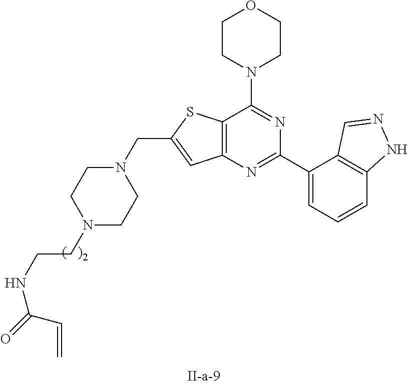

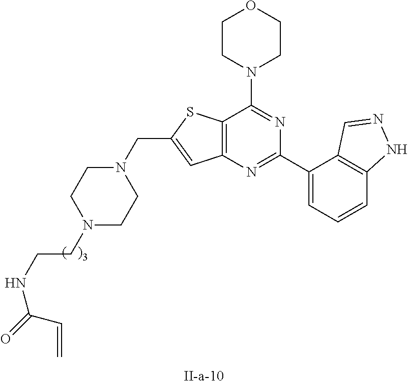

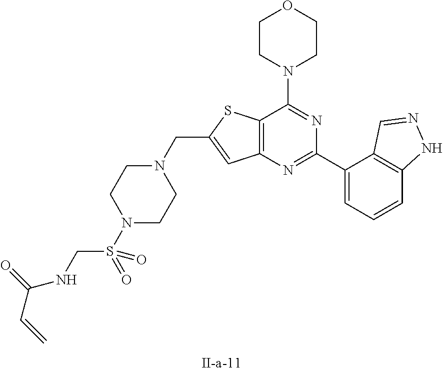

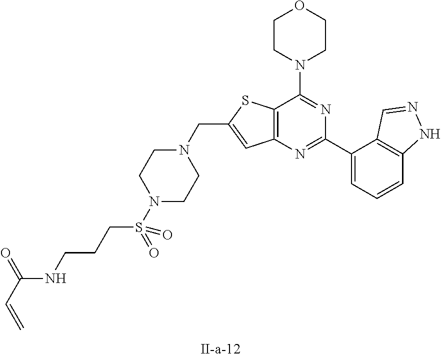

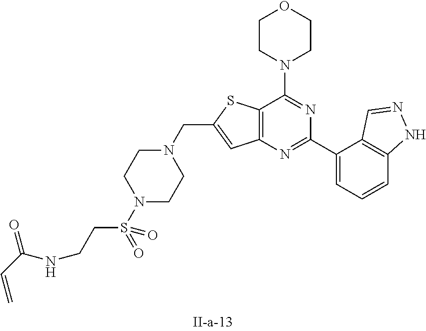

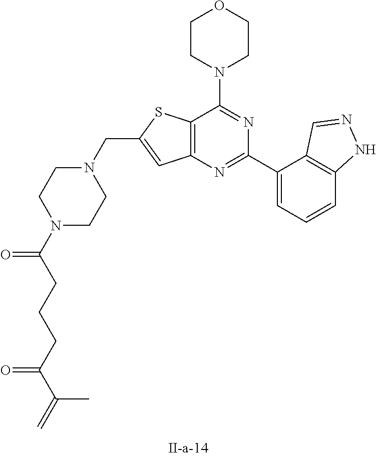

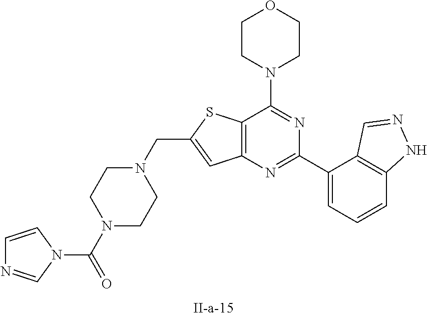

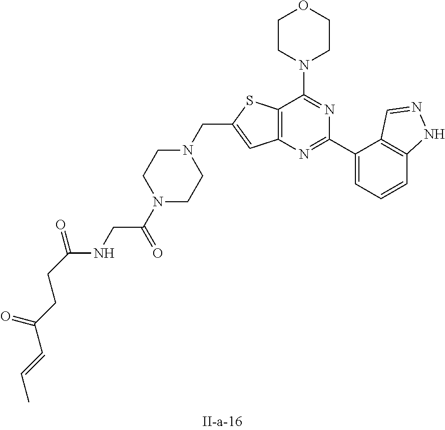

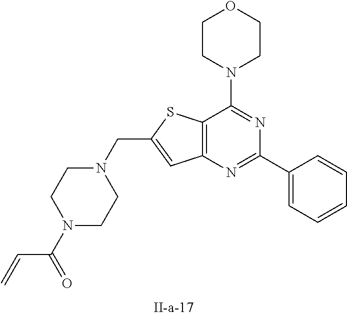

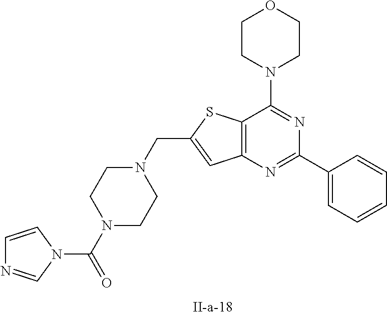

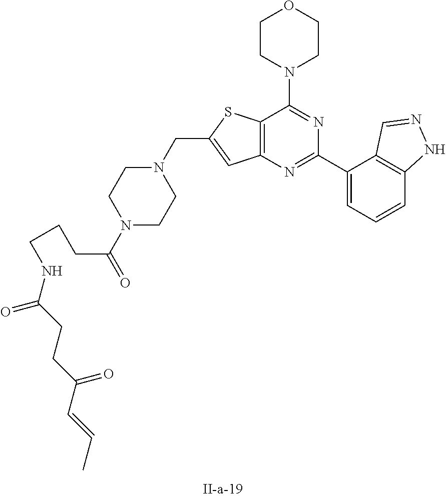

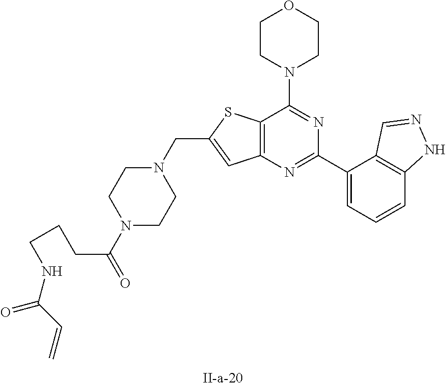

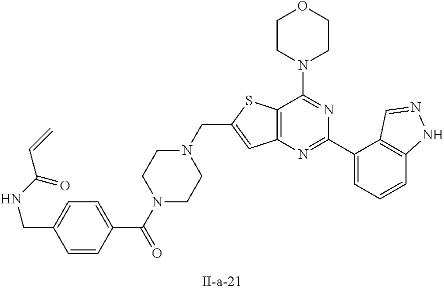

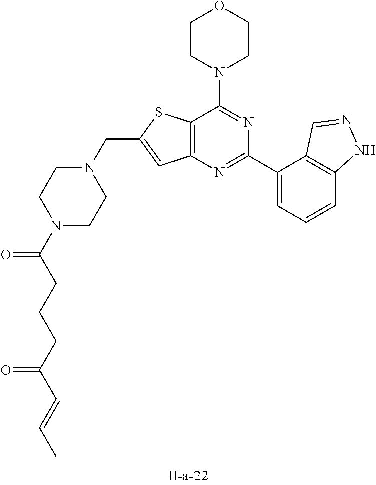

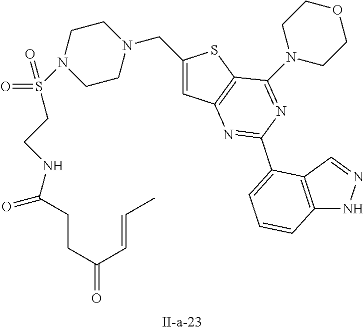

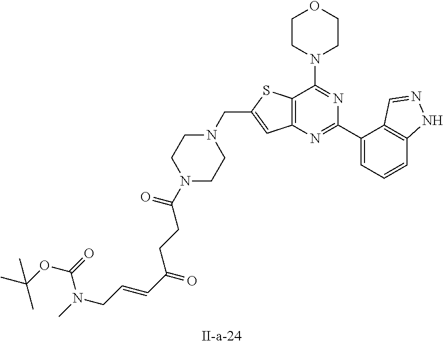

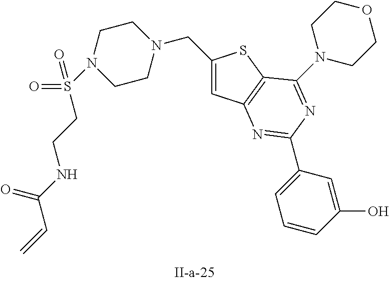

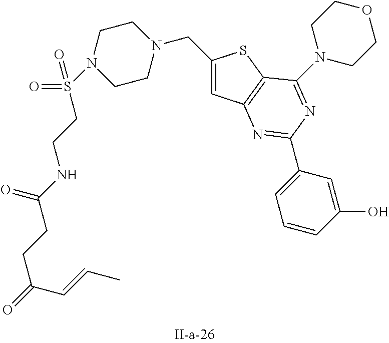

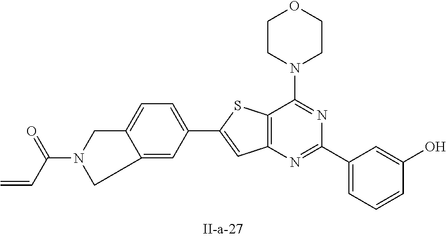

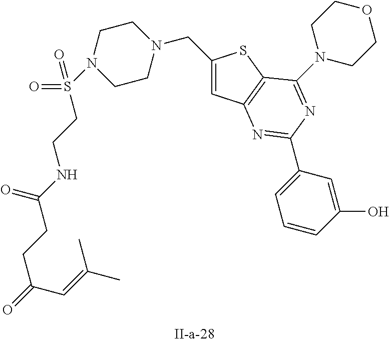

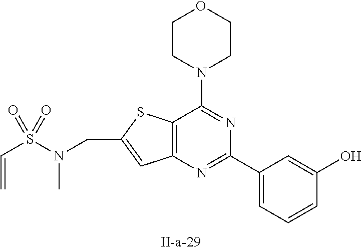

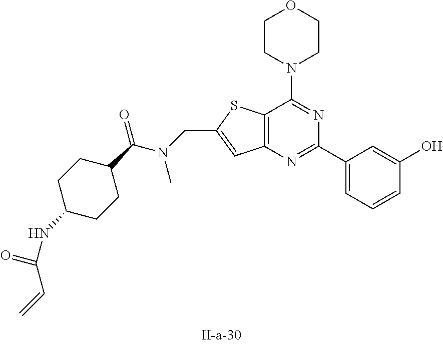

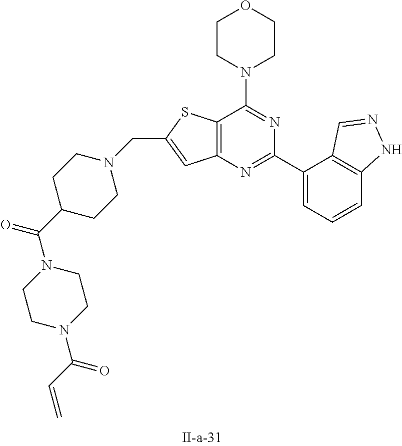

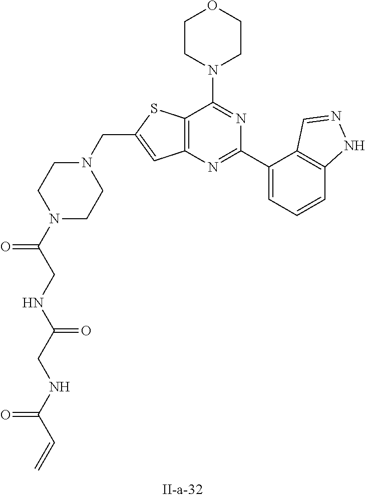

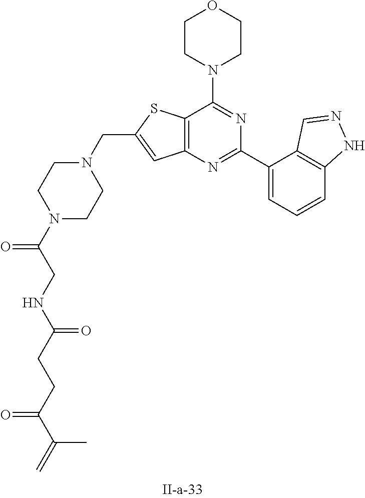

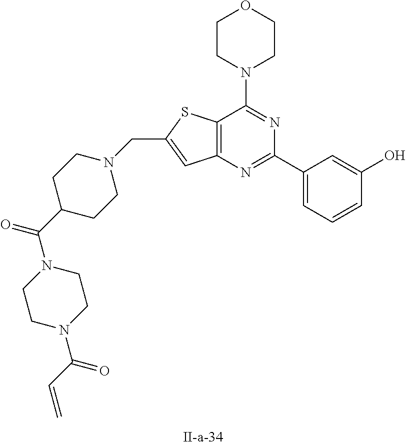

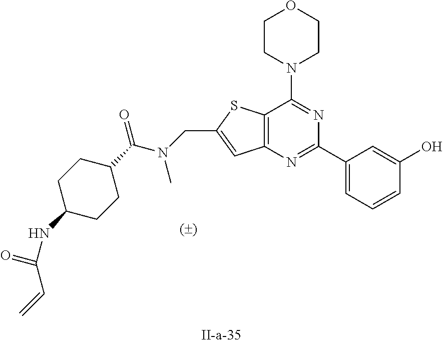

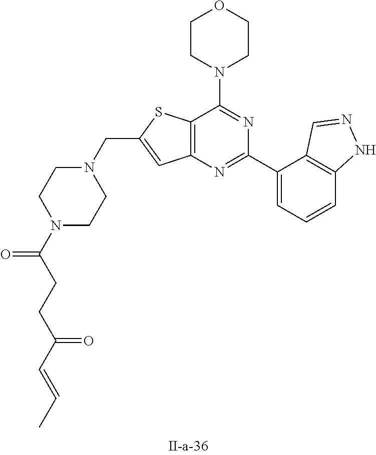

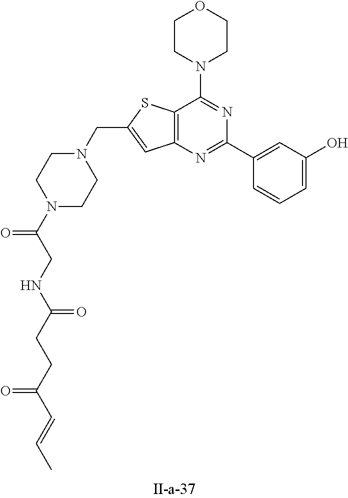

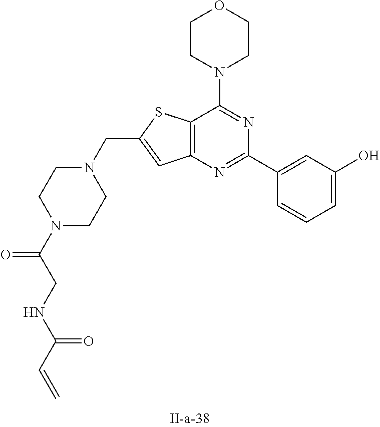

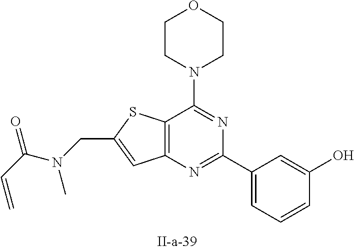

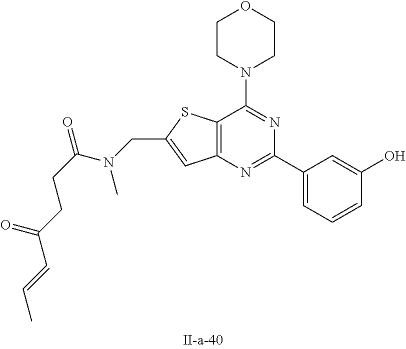

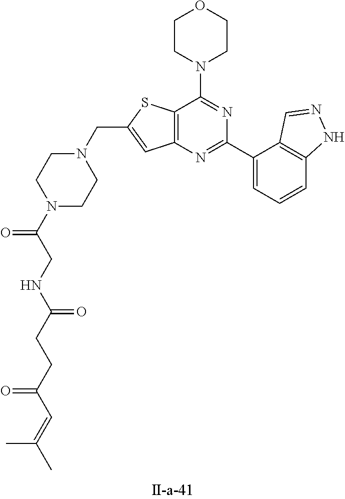

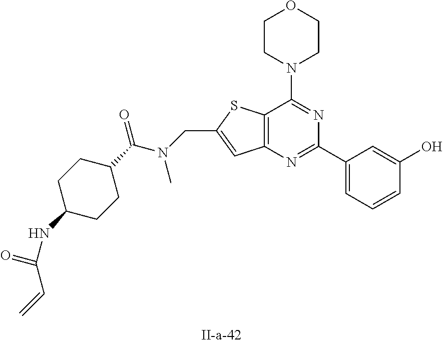

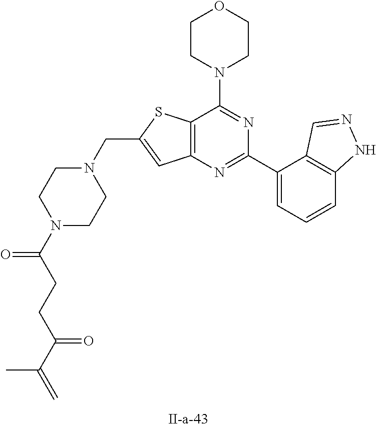

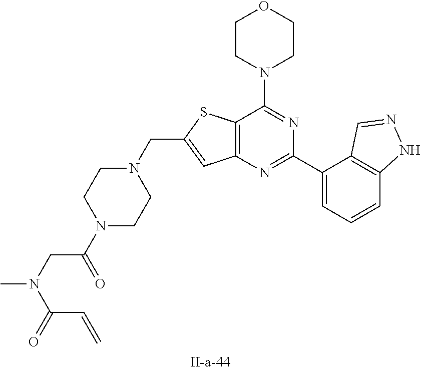

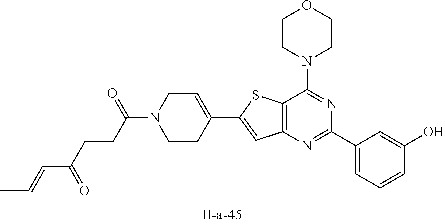

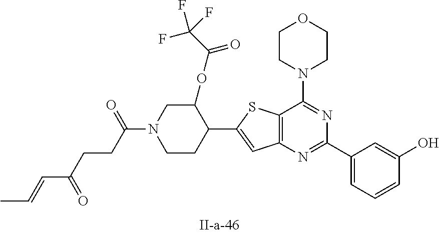

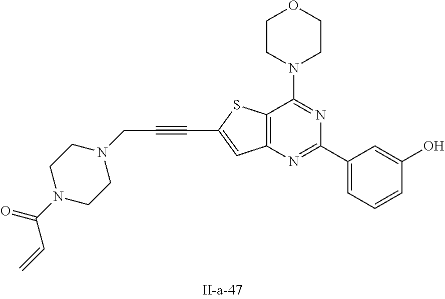

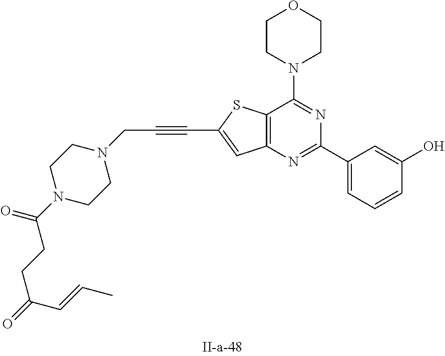

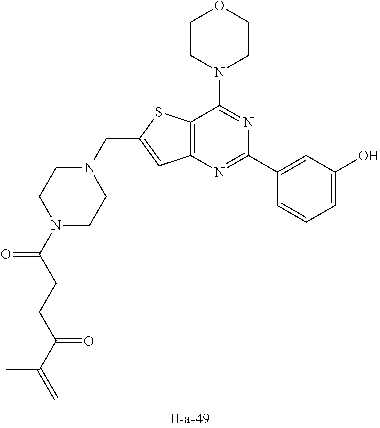

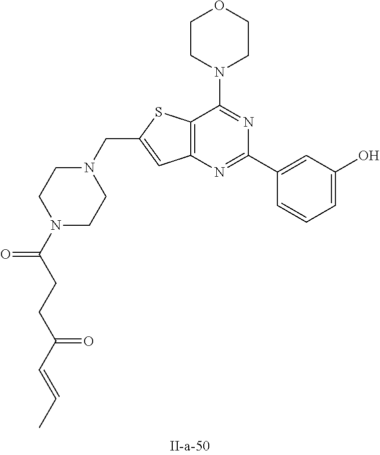

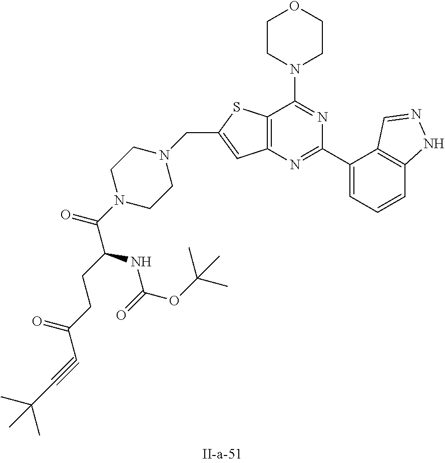

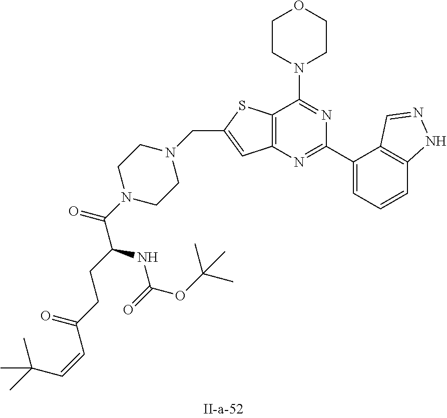

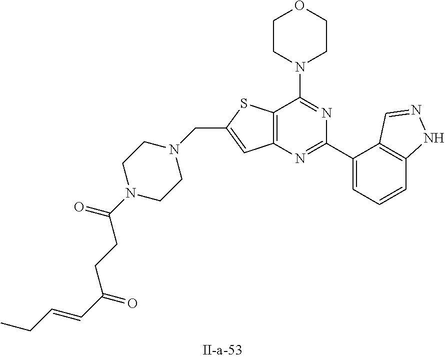

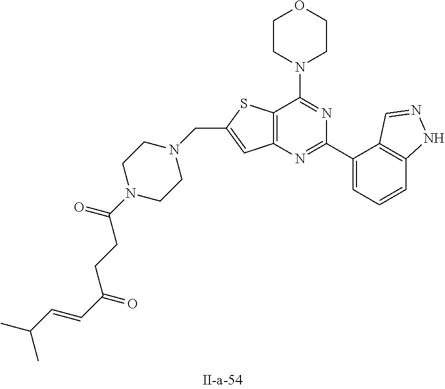

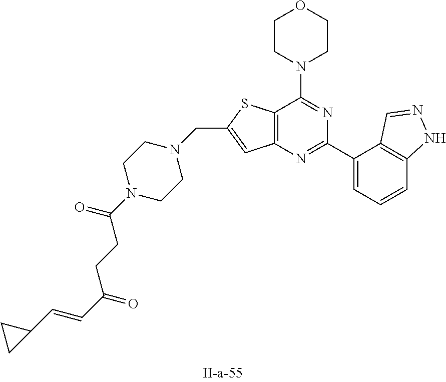

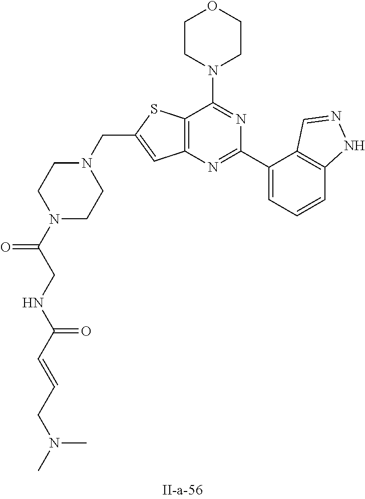

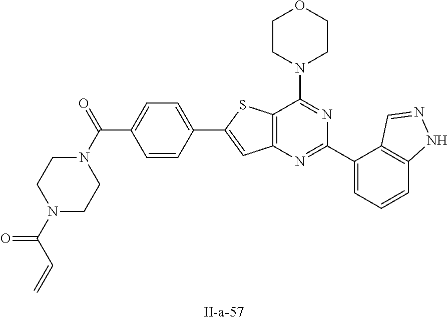

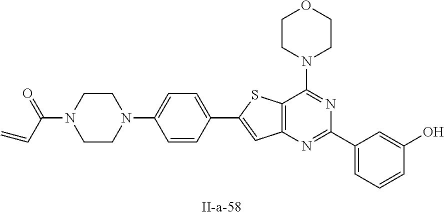

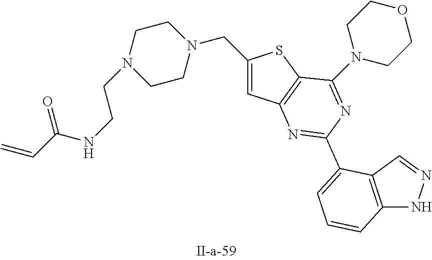

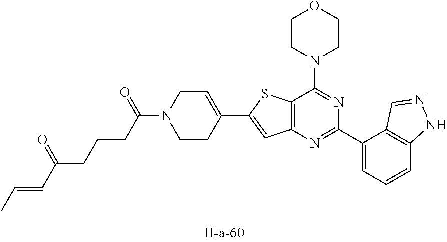

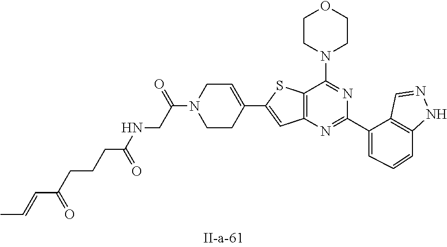

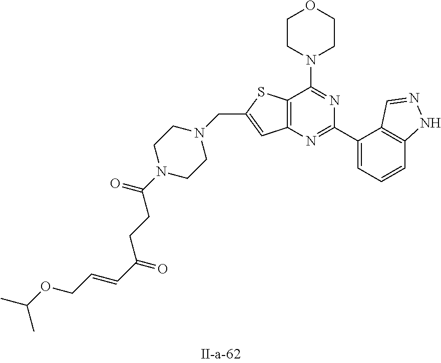

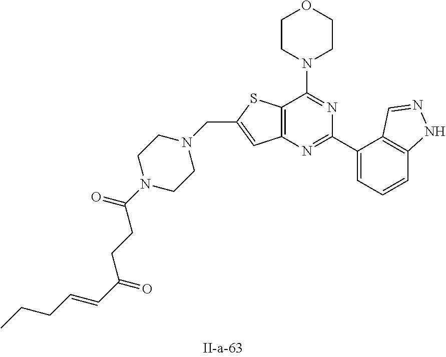

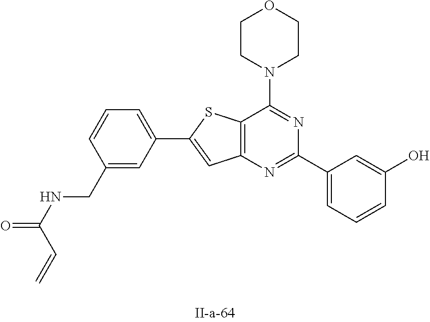

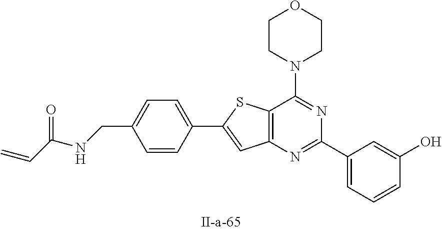

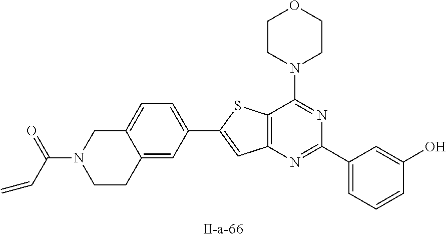

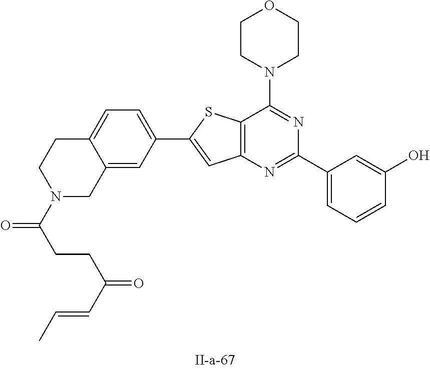

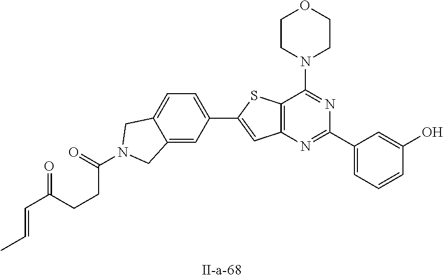

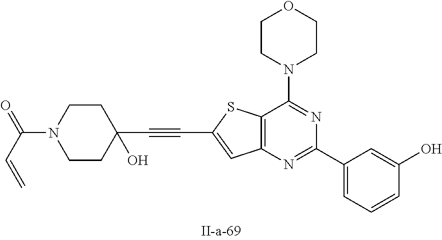

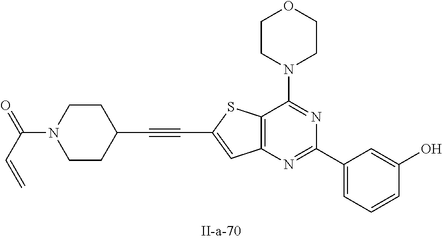

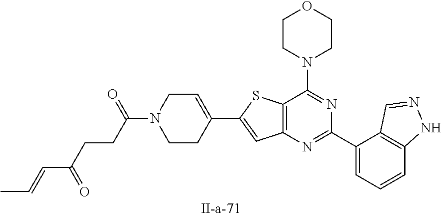

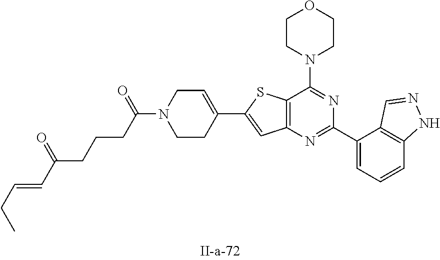

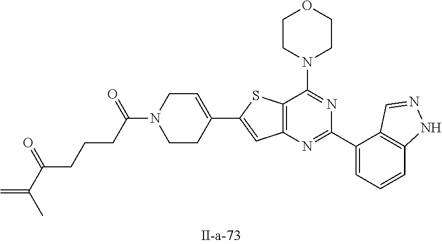

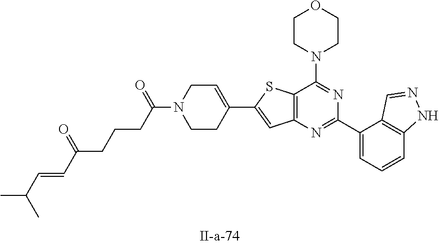

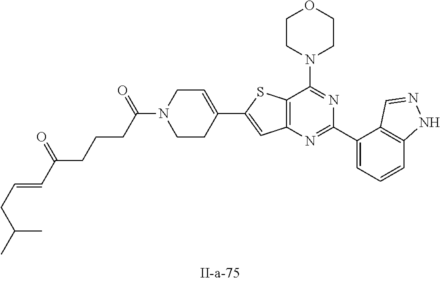

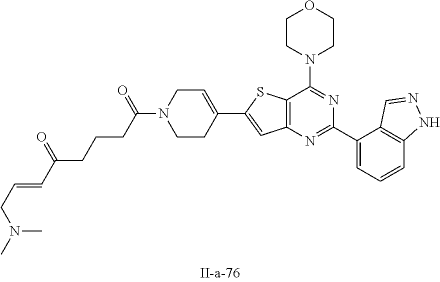

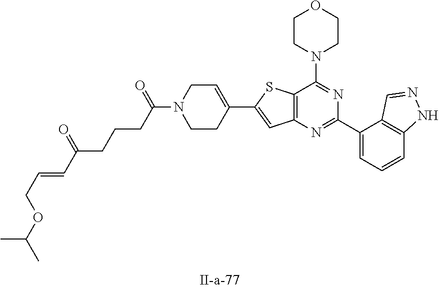

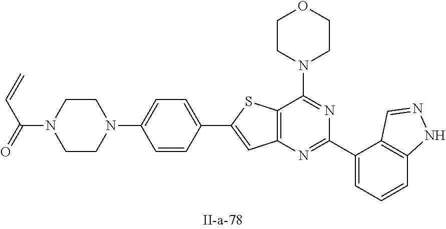

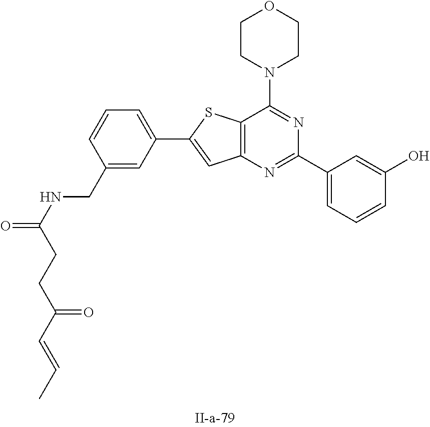

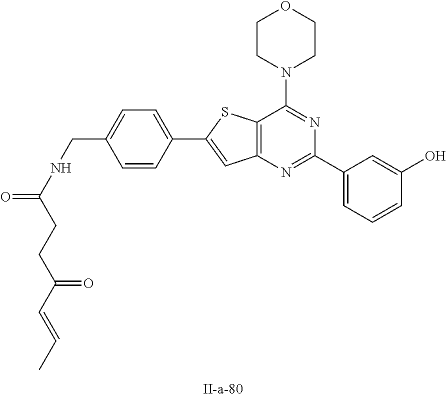

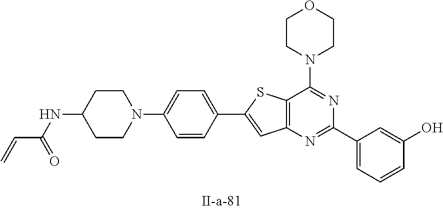

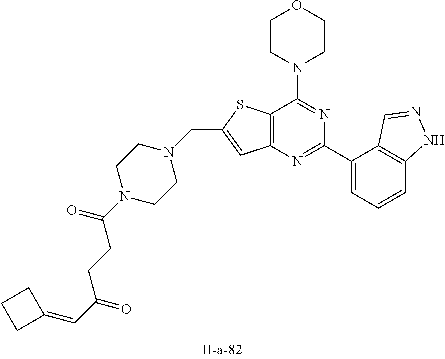

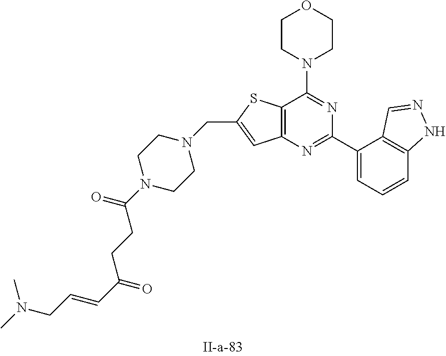

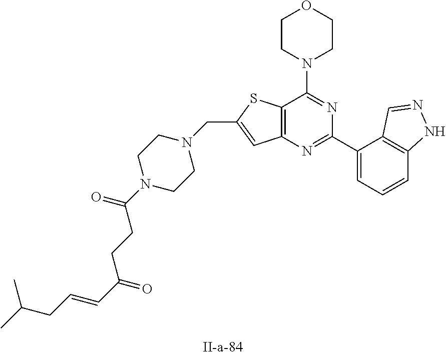

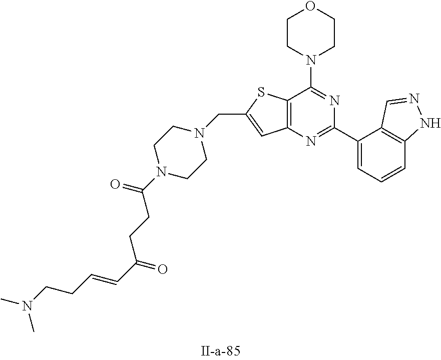

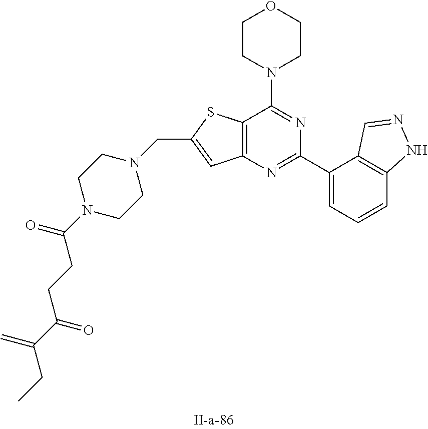

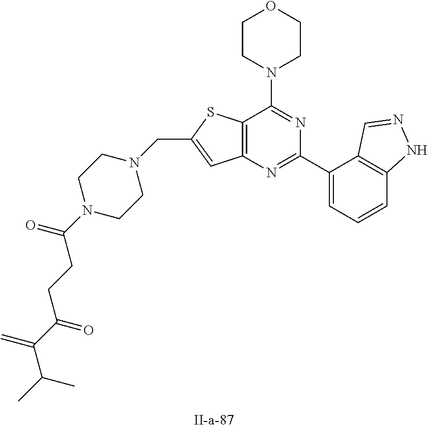

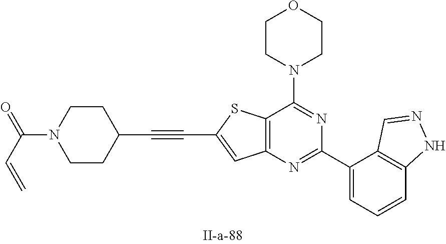

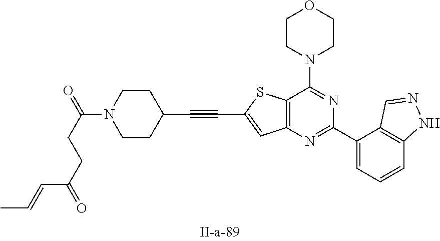

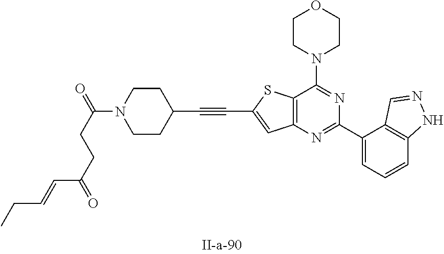

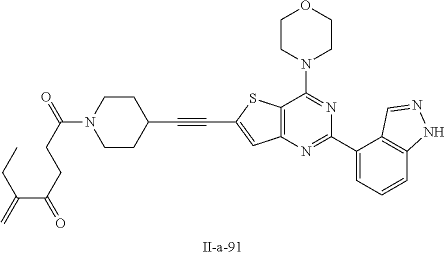

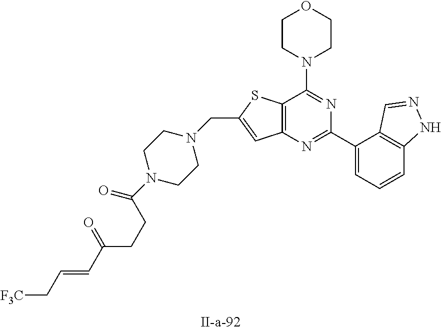

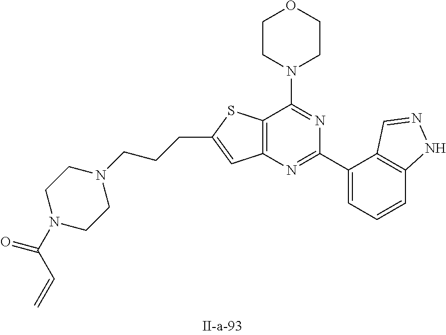

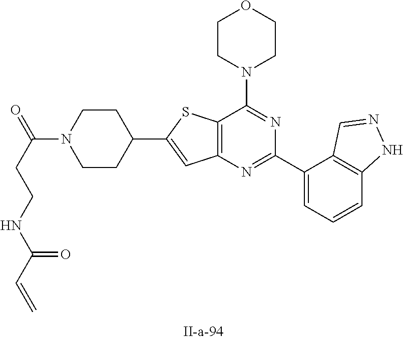

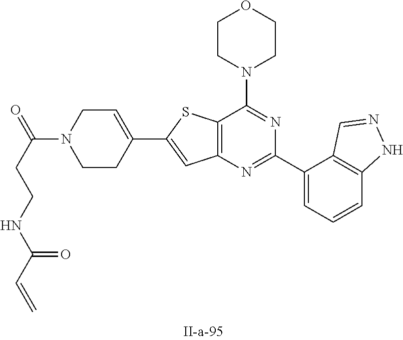

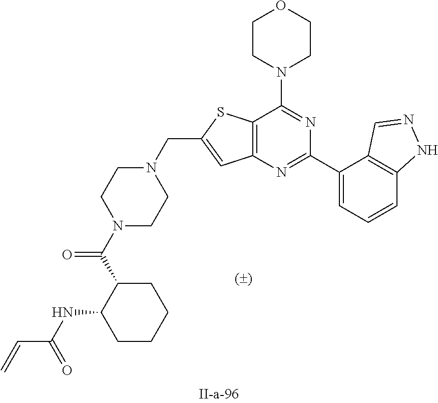

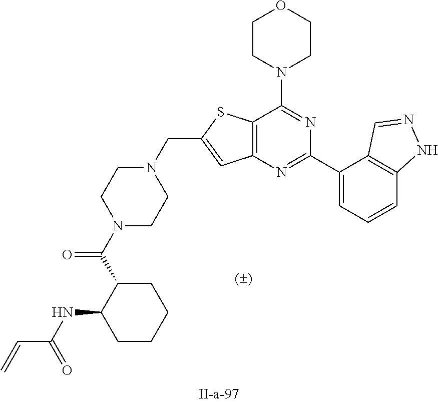

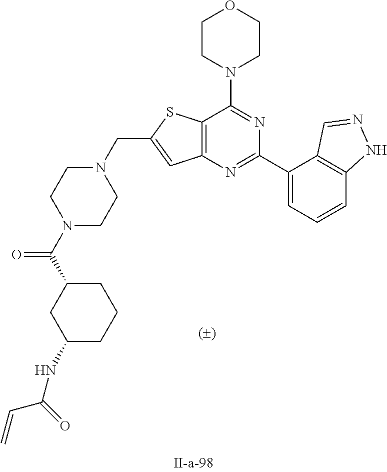

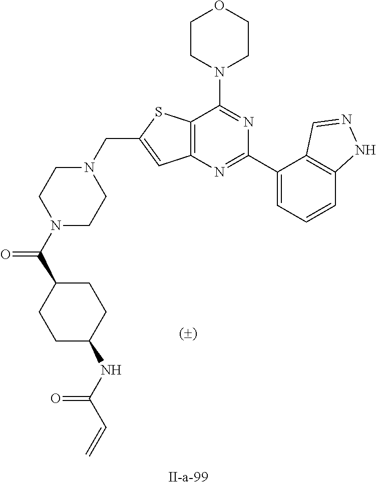

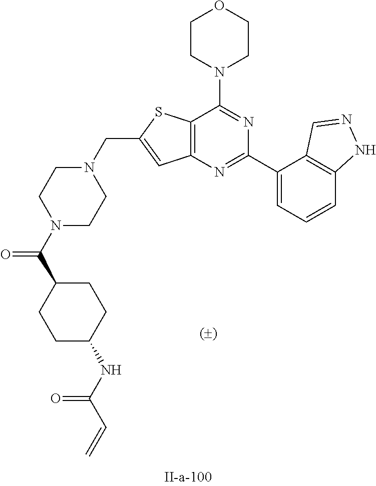

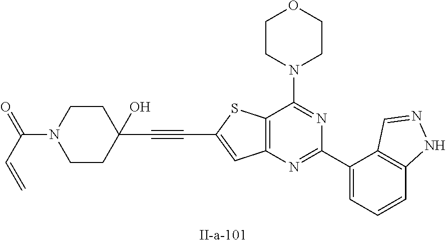

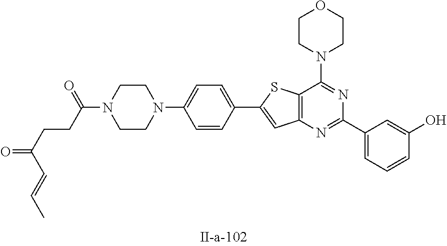

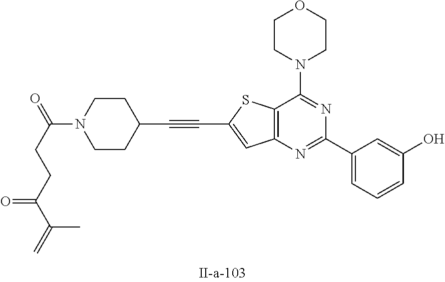

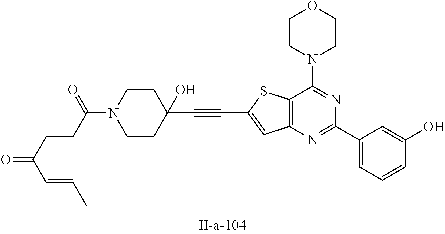

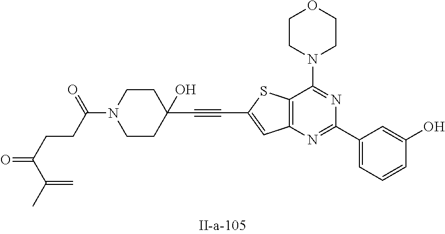

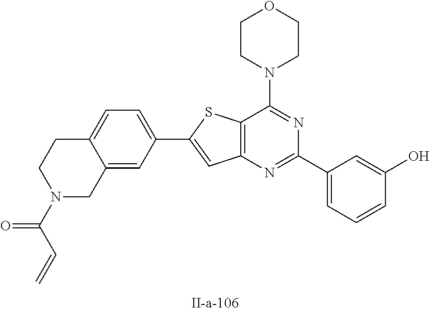

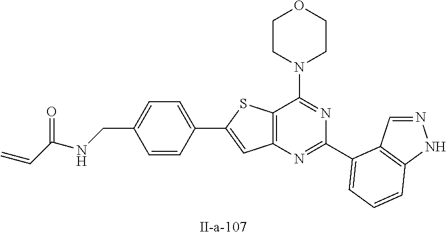

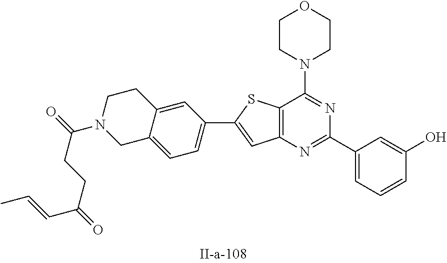

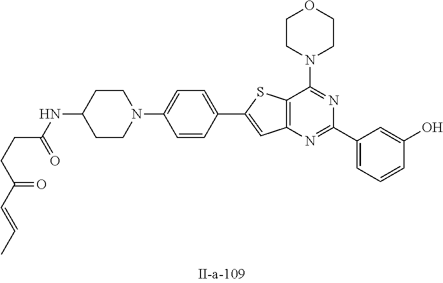

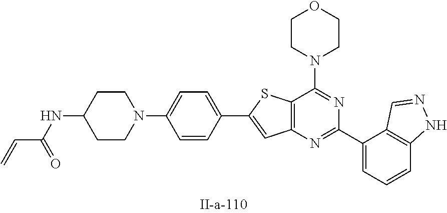

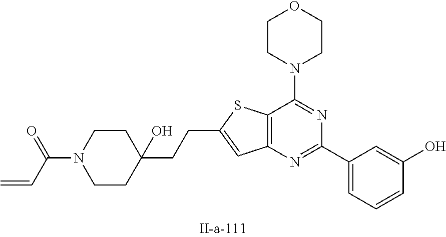

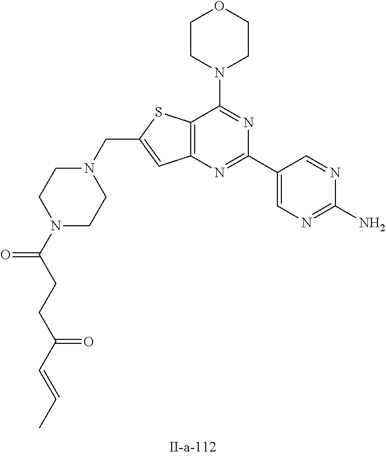

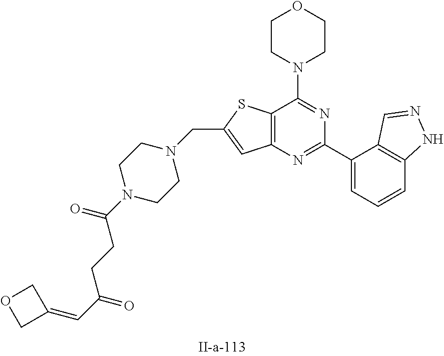

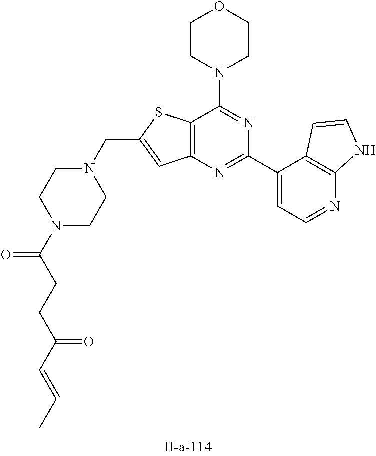

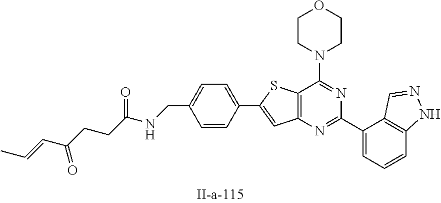

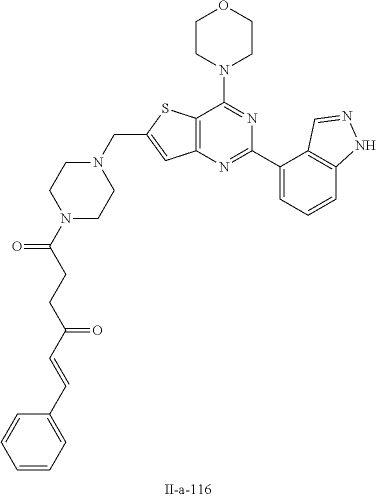

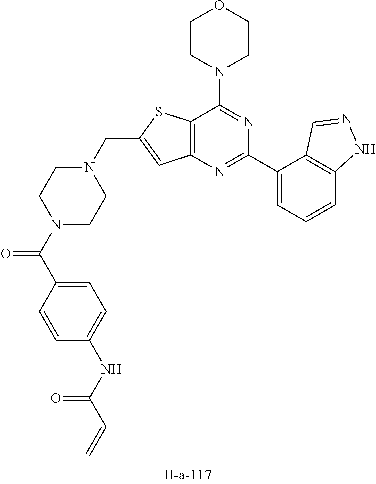

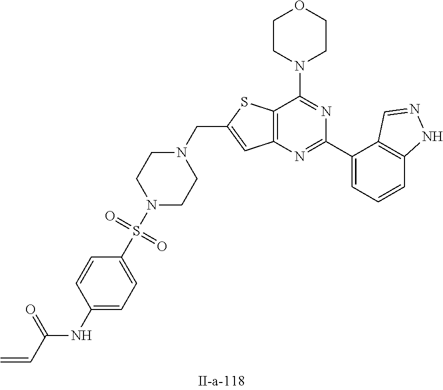

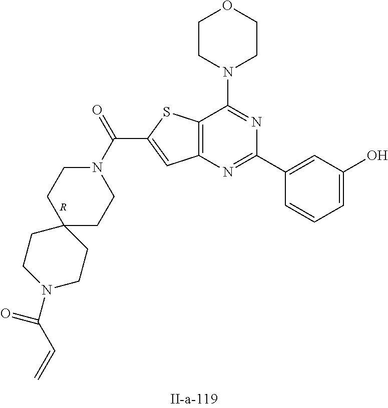

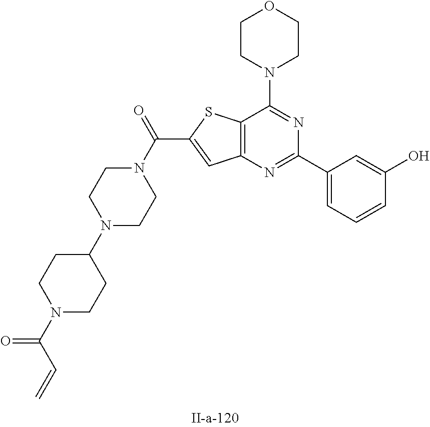

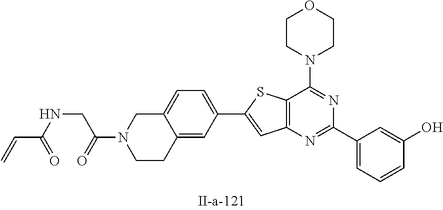

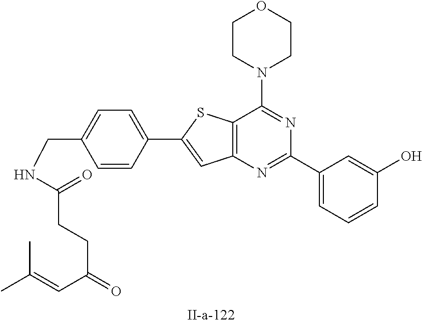

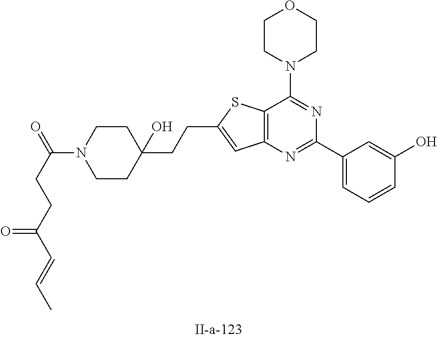

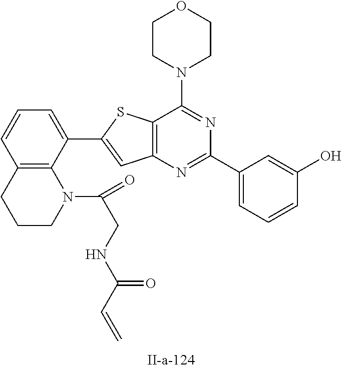

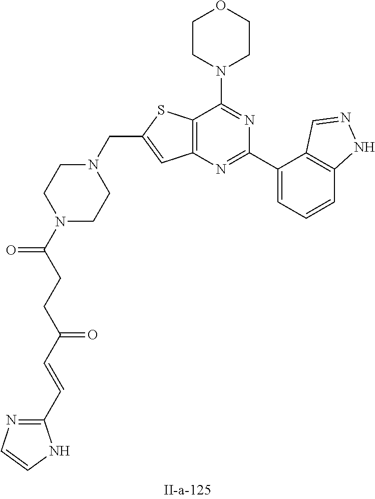

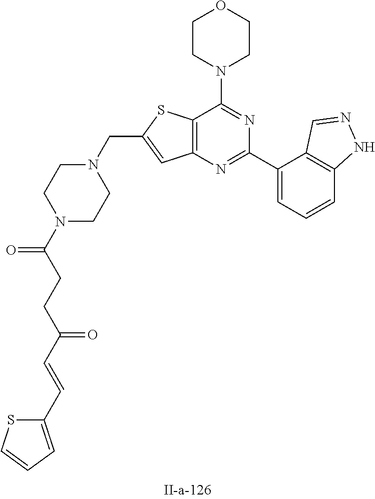

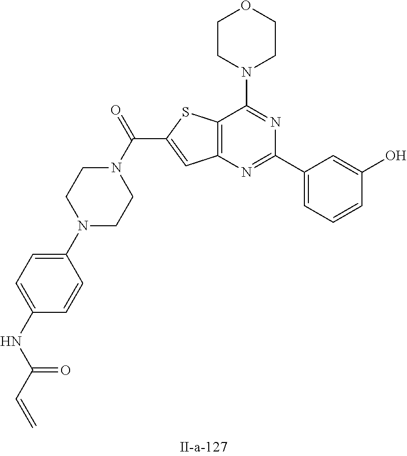

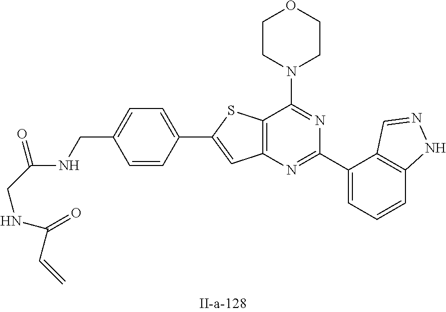

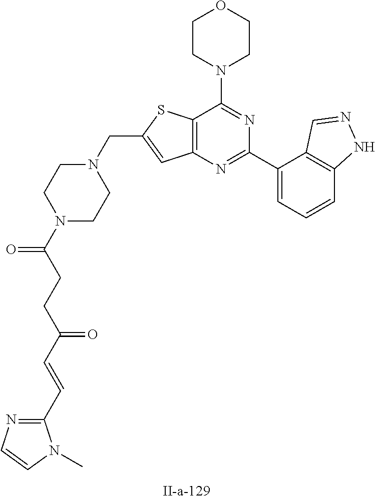

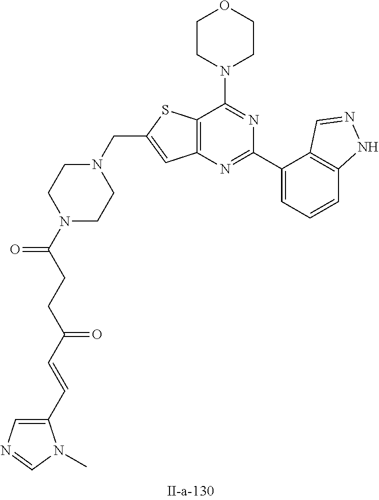

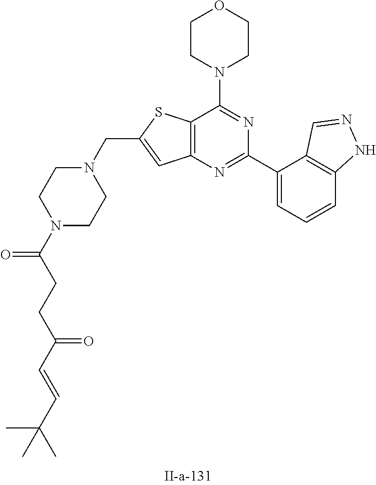

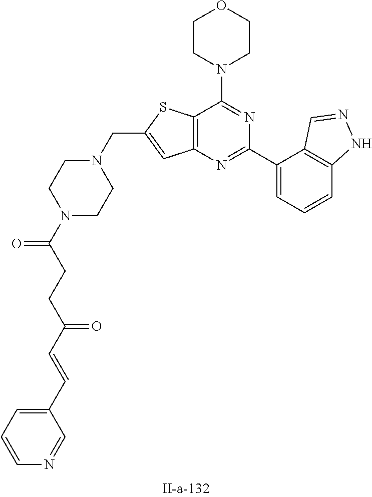

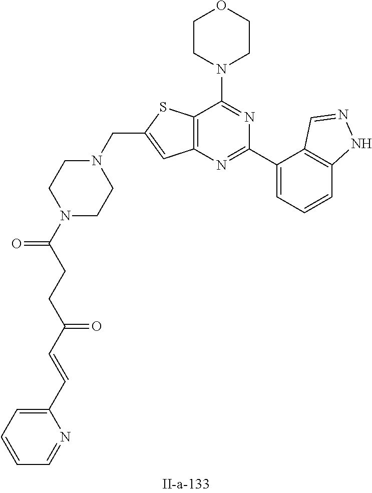

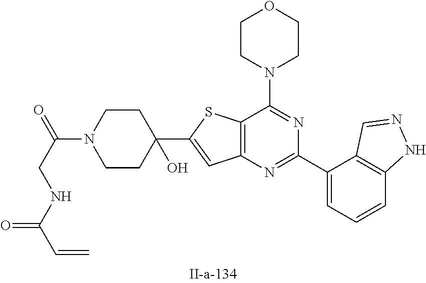

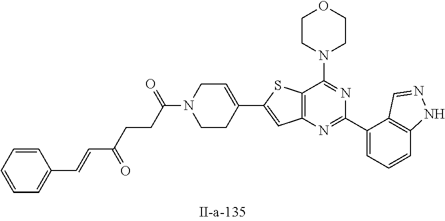

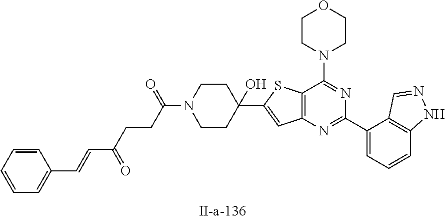

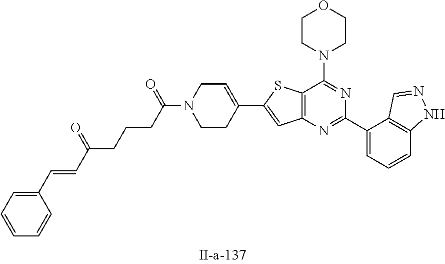

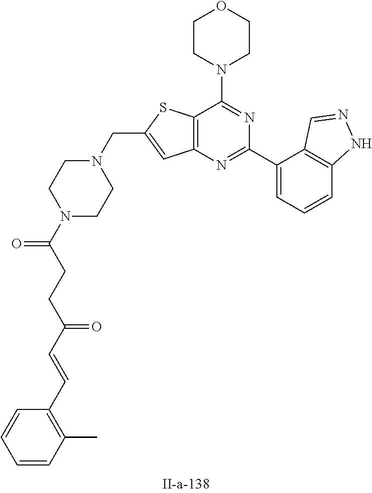

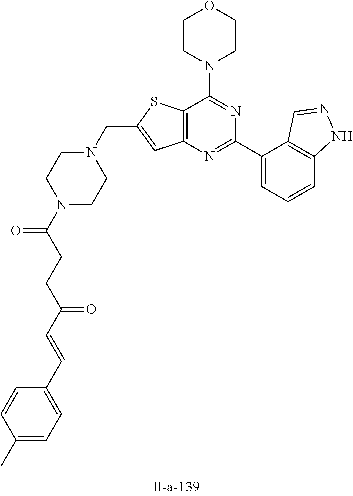

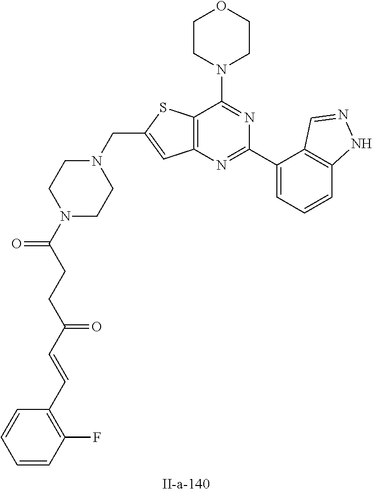

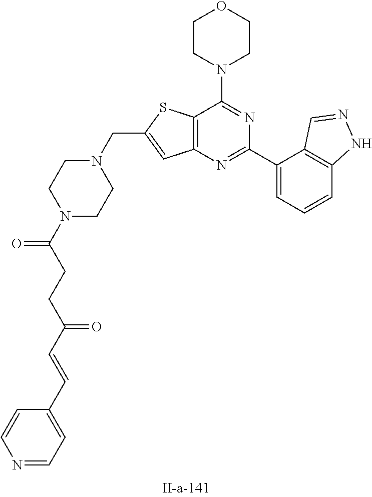

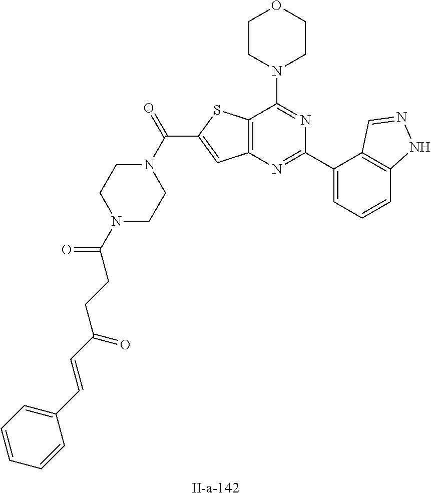

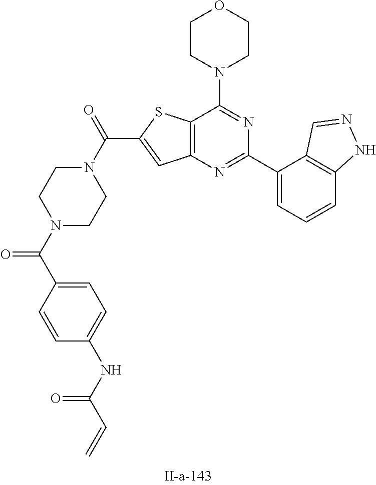

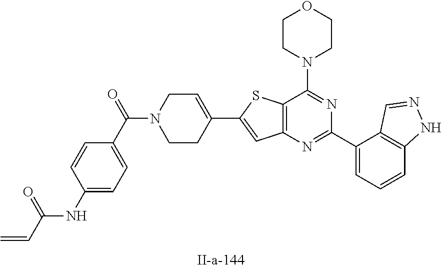

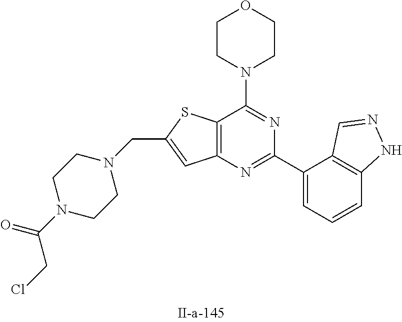

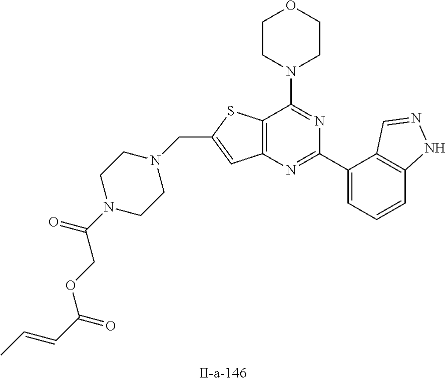

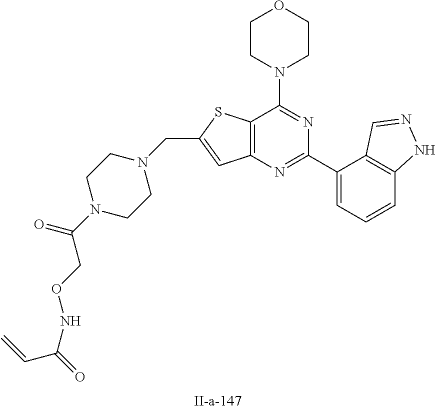

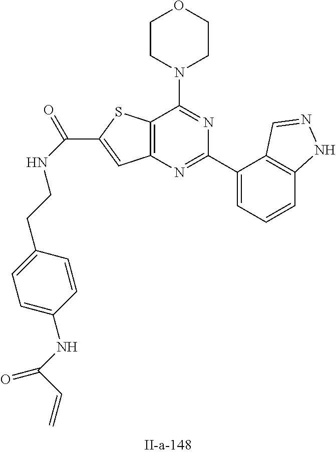

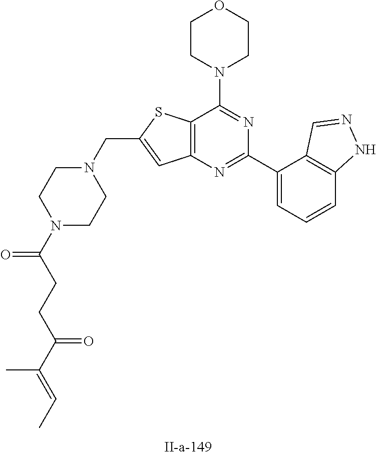

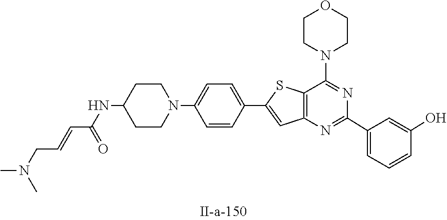

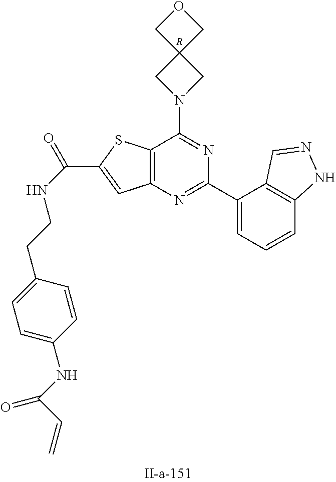

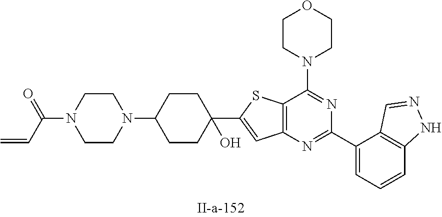

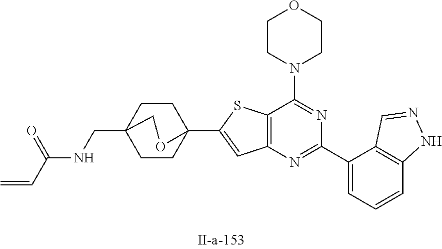

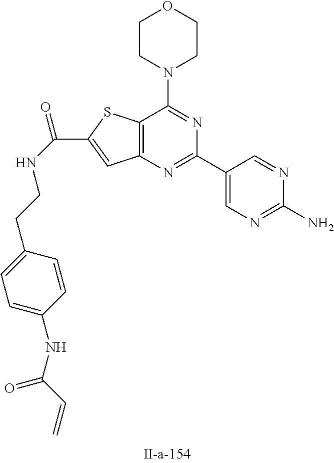

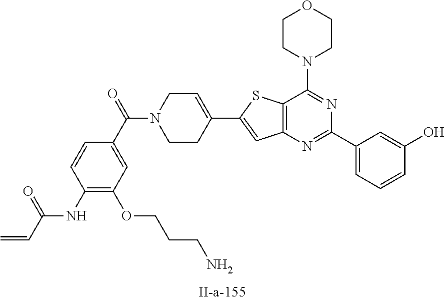

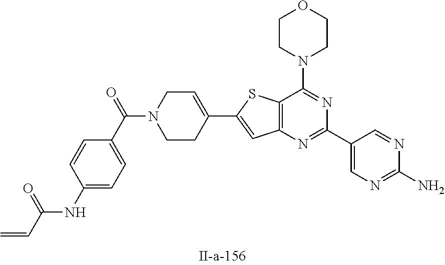

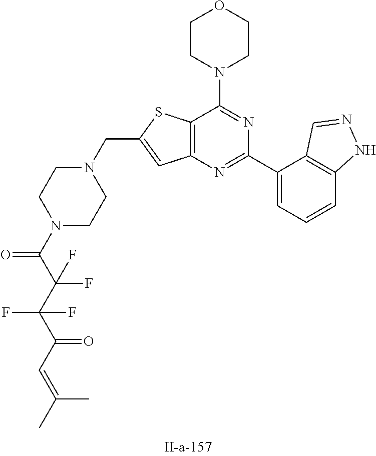

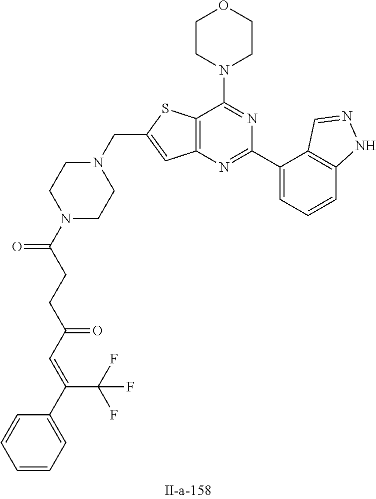

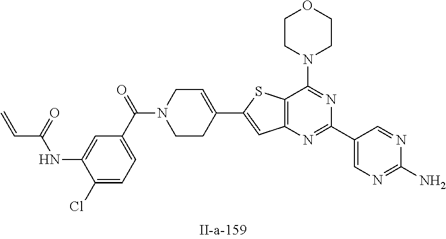

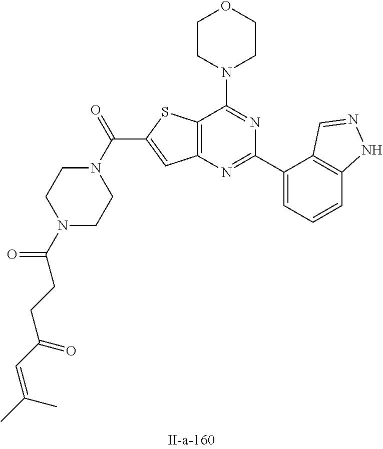

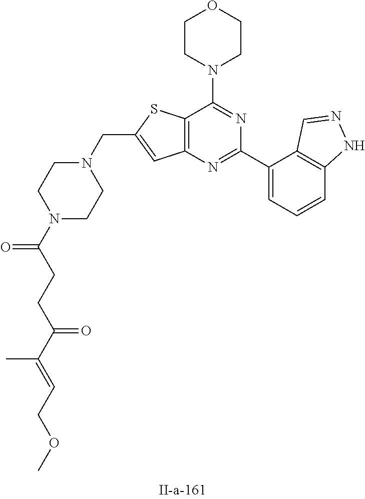

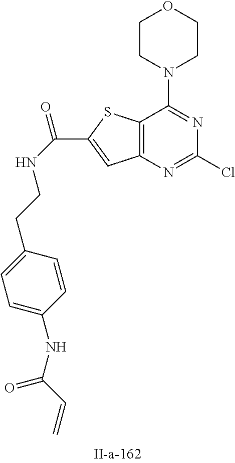

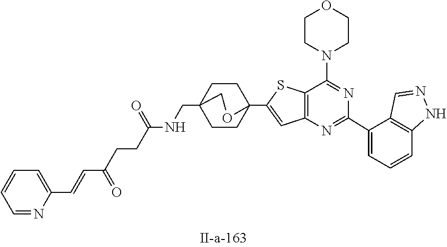

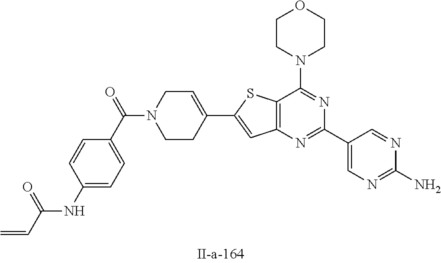

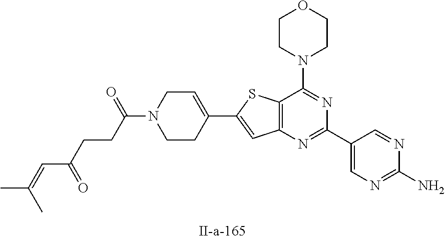

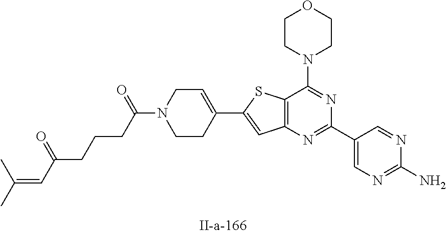

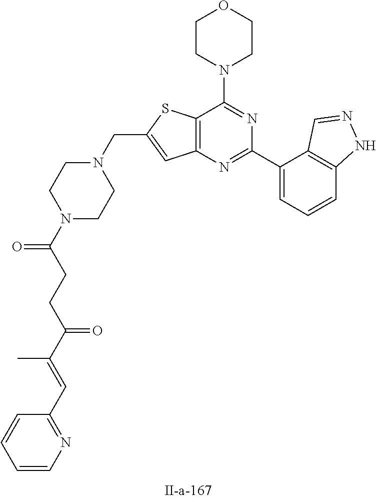

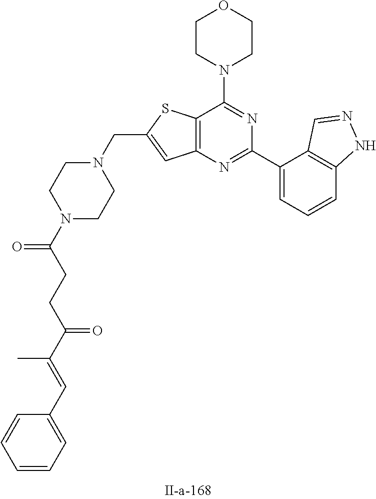

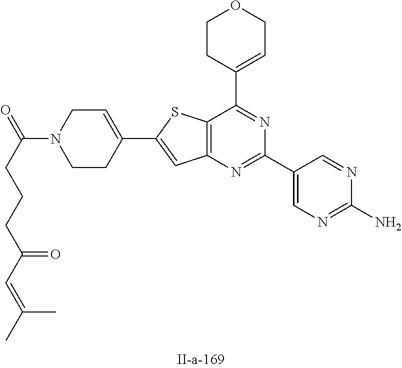

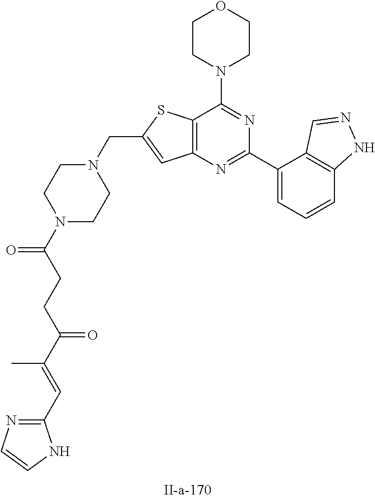

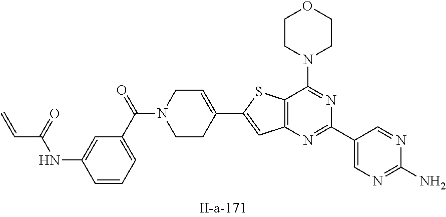

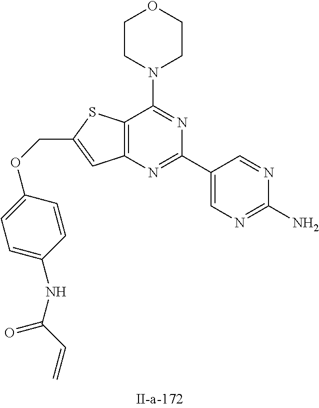

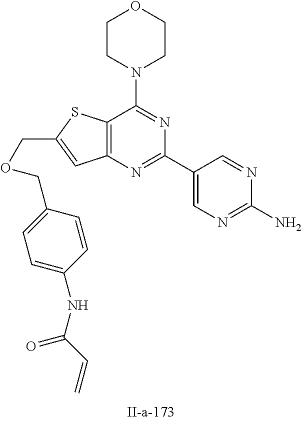

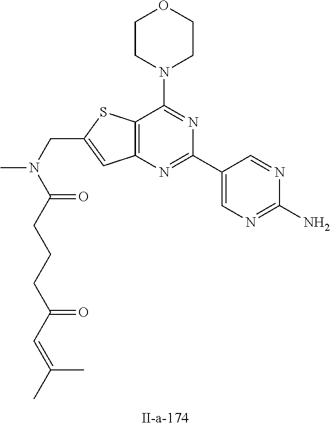

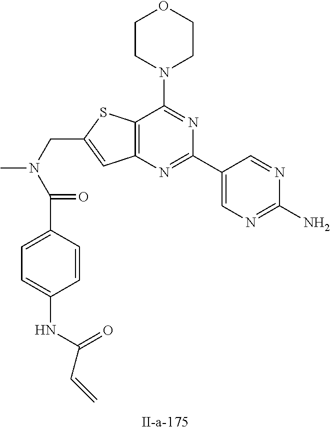

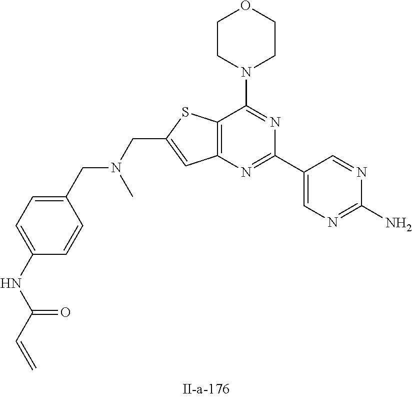

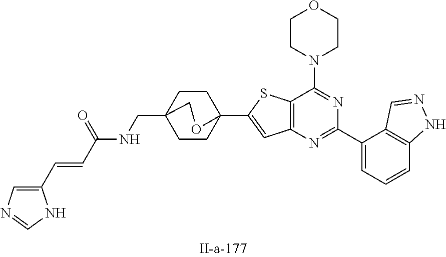

In certain embodiments, the present invention provides any compound selected from those depicted in Table 6, above, or a pharmaceutically acceptable salt thereof.

Exemplary compounds of formula II-c are set forth in Table 7, below:

TABLE 7
Exemplary Compounds of Formula II-c 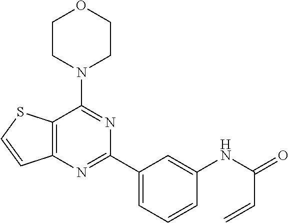
II-c-1 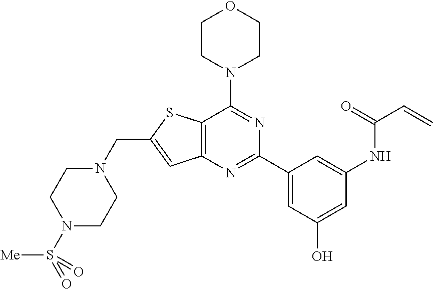
II-c-2 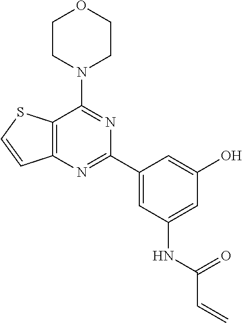
II-c-3 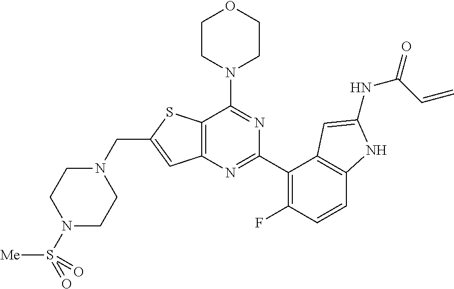
II-c-4 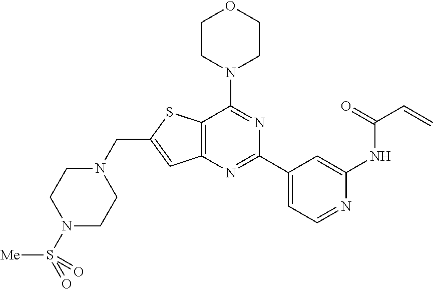
II-c-5 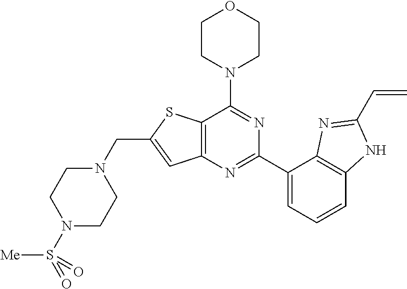
II-c-6 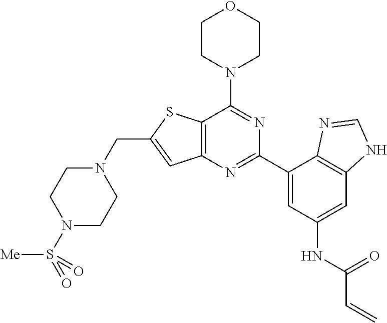
II-c-7

In certain embodiments, the present invention provides any compound selected from those depicted in Table 7, above, or a pharmaceutically acceptable salt thereof.

Exemplary compounds of formula II-g are set forth in Table 8, below:

TABLE 8
Exemplary Compounds of Formula II-g 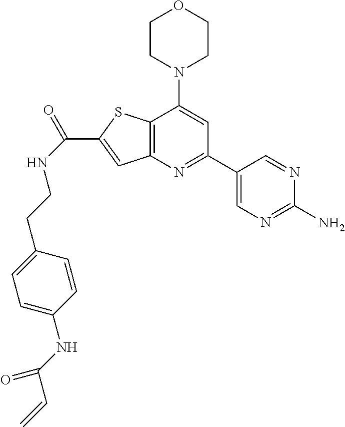
II-g-1 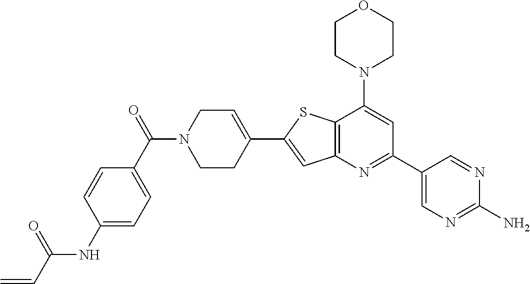
II-g-2 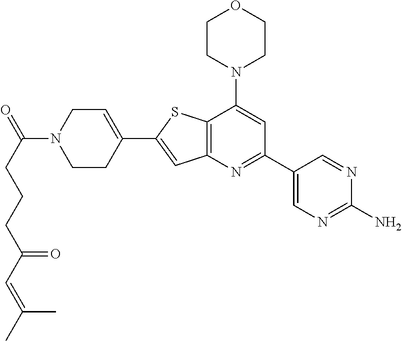
II-g-3 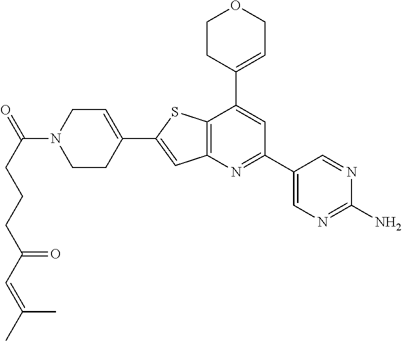
II-g-4 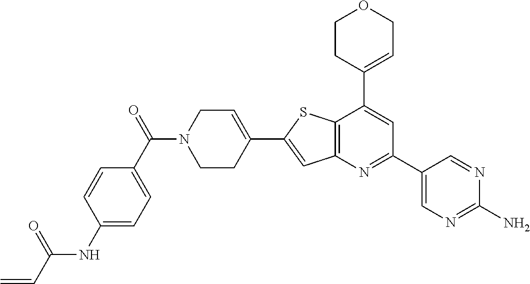
II-g-5 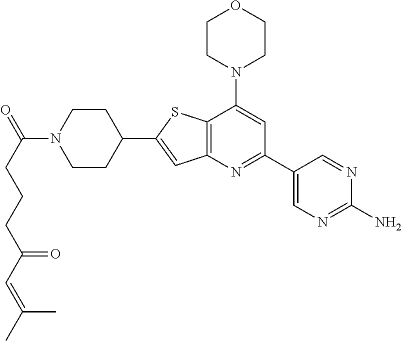
II-g-6 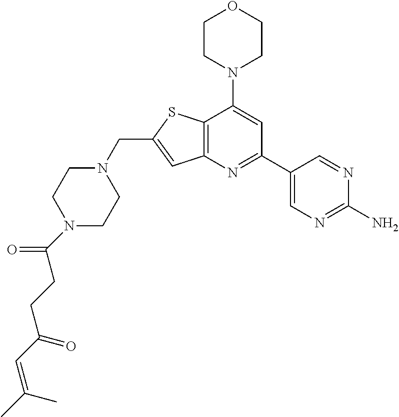
II-g-7 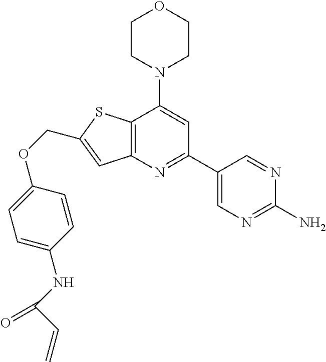
II-g-8

In certain embodiments, the present invention provides any compound selected from those depicted in Table 8, above, or a pharmaceutically acceptable salt thereof.

Exemplary compounds of formula III are set forth in Table 9, below:

TABLE 9
Exemplary Compounds of Formula III 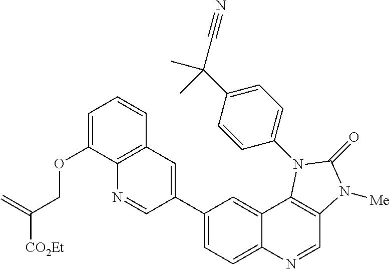
III-1 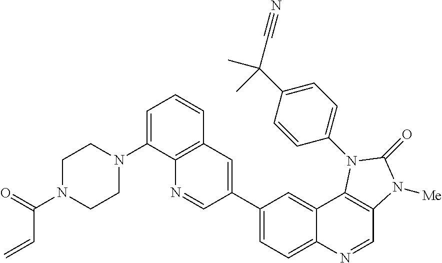
III-2 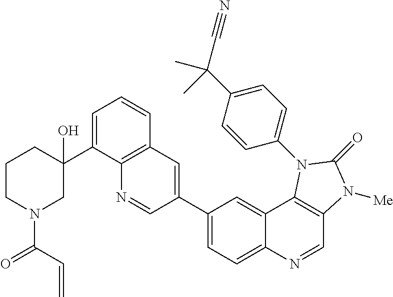
III-3 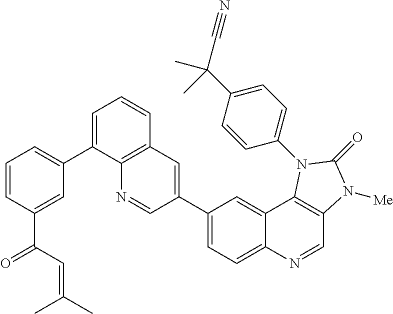
III-4 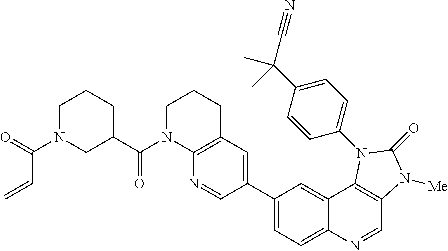
III-5 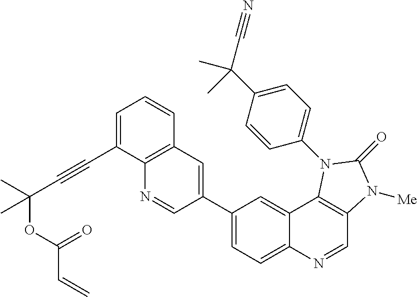
III-6 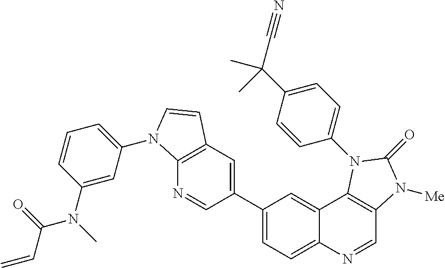
III-7 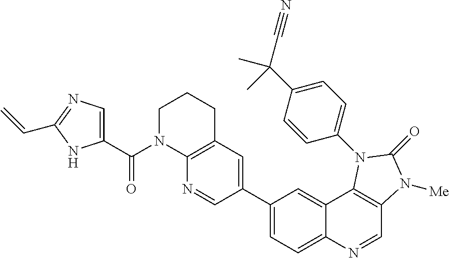
III-8 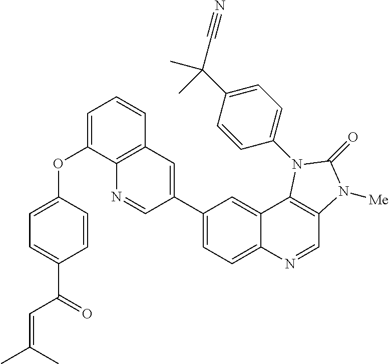
III-9 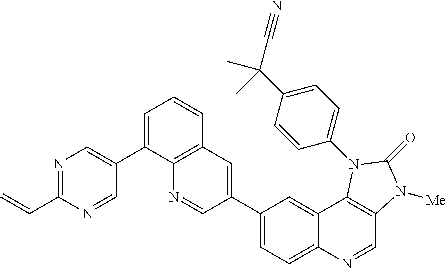
III-10 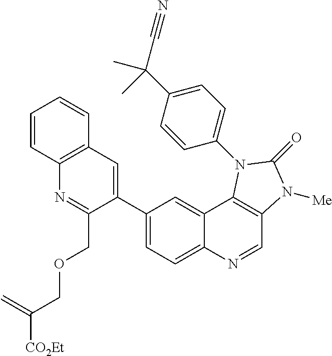
III-11 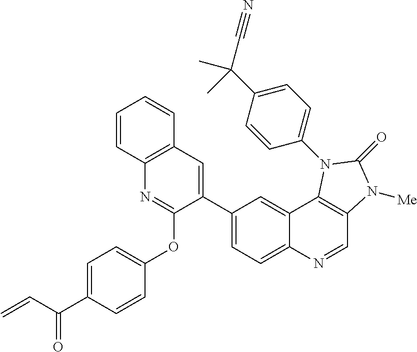
III-12 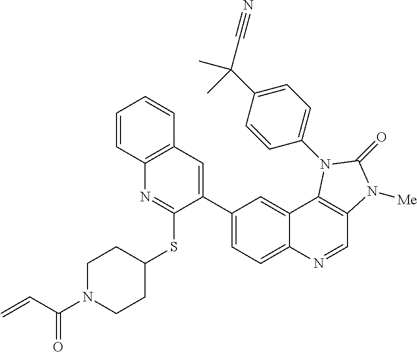
III-13 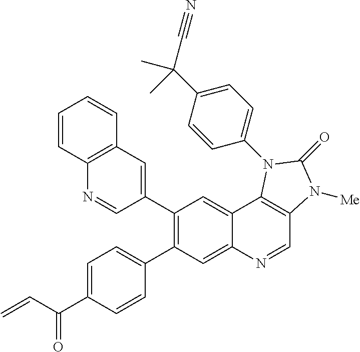
III-14 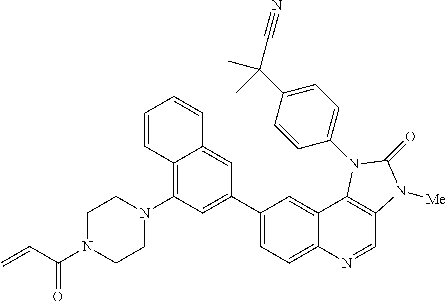
III-15 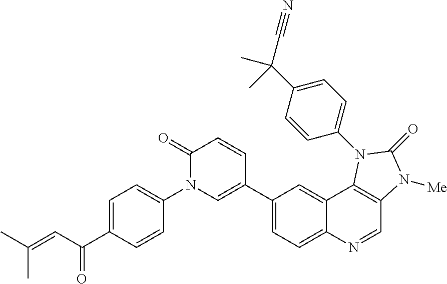
III-16 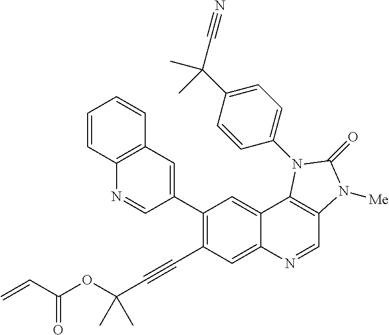
III-17

In certain embodiments, the present invention provides any compound selected from those depicted in Table 9, above, or a pharmaceutically acceptable salt thereof.

Exemplary compounds of formula V are set forth in Table 10, below:

TABLE 10
Exemplary Compounds of Formula V 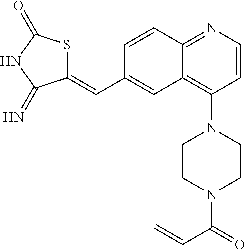
V-1 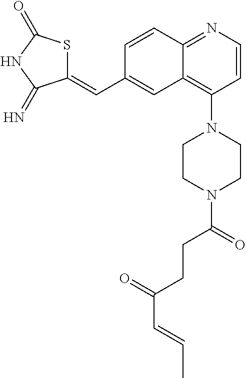
V-2 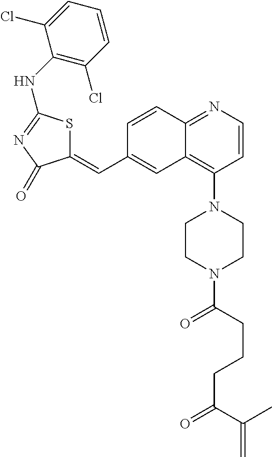
V-3 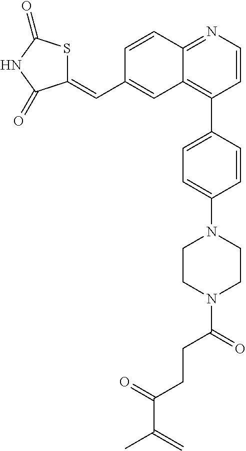
V-4 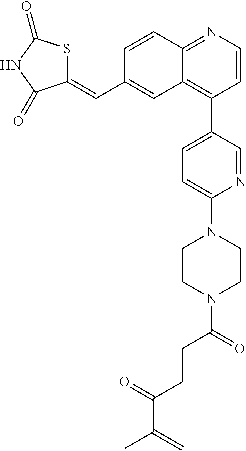
V-5 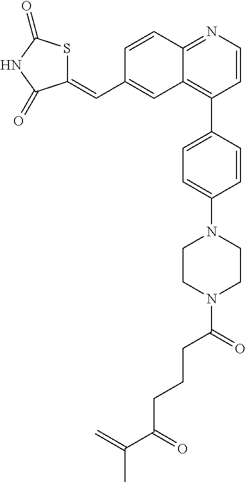
V-6 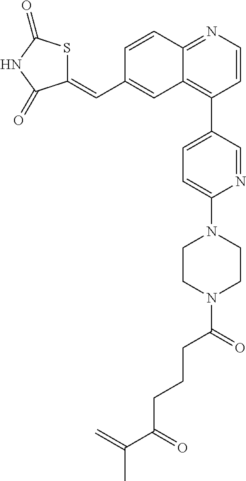
V-7 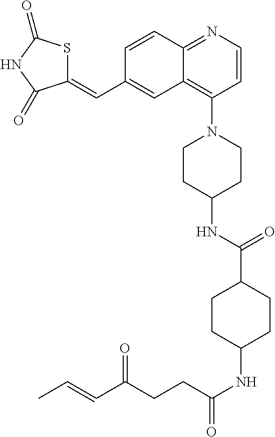
V-8 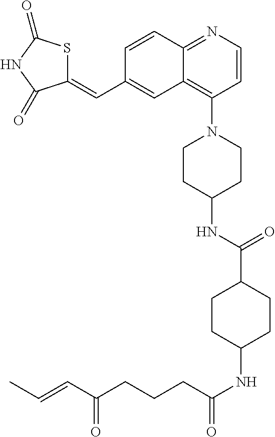
V-9 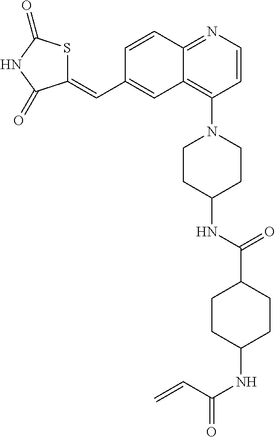
V-10 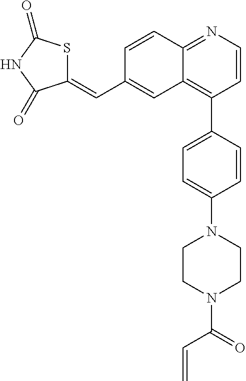
V-11 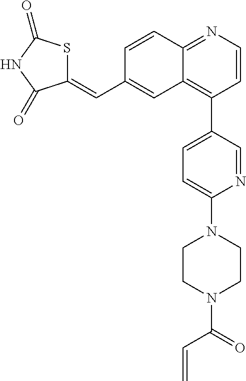
V-12 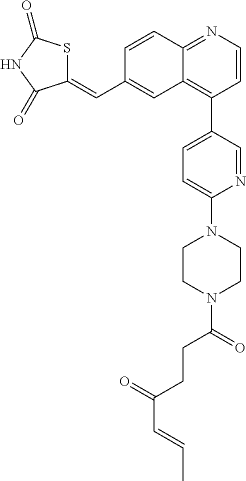
V-13 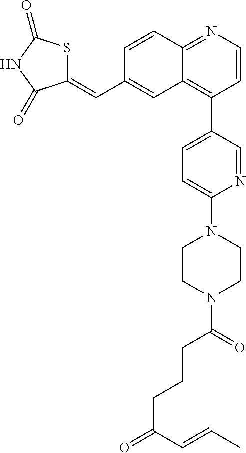
V-14 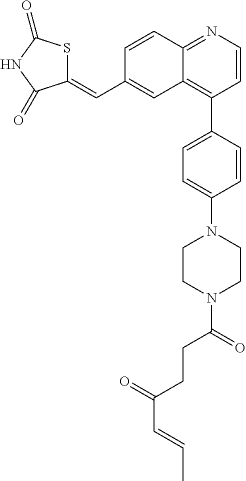
V-15 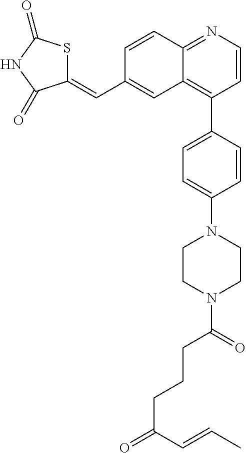
V-16 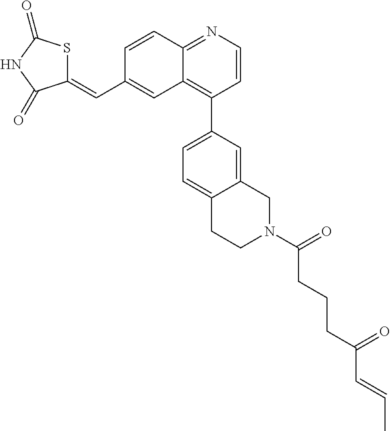
V-17 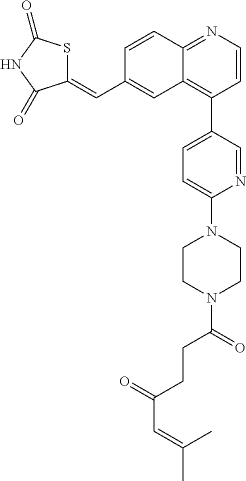
V-18 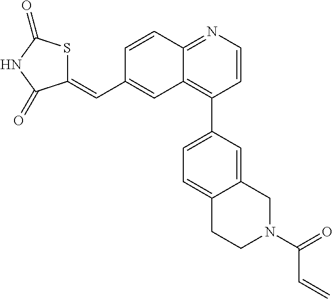
V-19 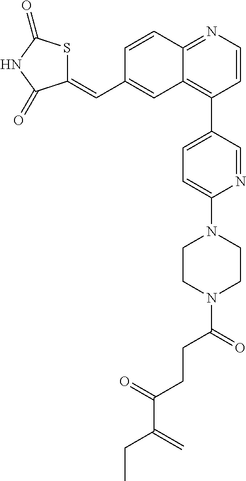
V-20

In certain embodiments, the present invention provides any compound selected from those depicted in Table 10, above, or a pharmaceutically acceptable salt thereof.

Exemplary compounds of formula VI are set forth in Table 11, below:

TABLE 11
Exemplary Compounds of Formula VI 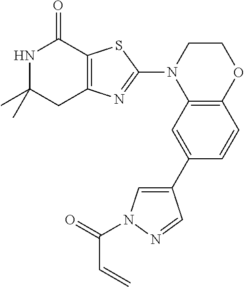
VI-1 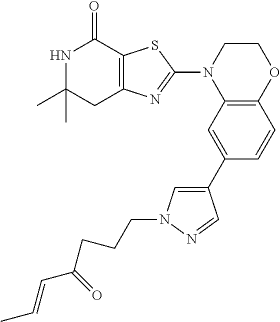
VI-2 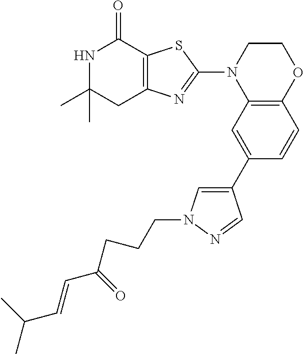
VI-3 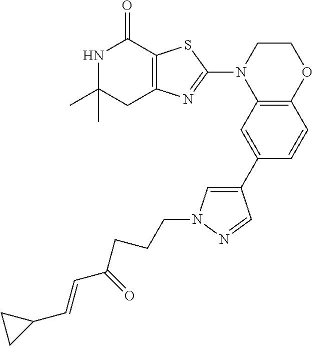
VI-4 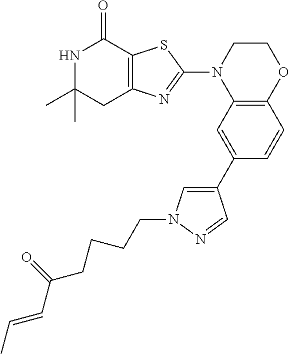
VI-5 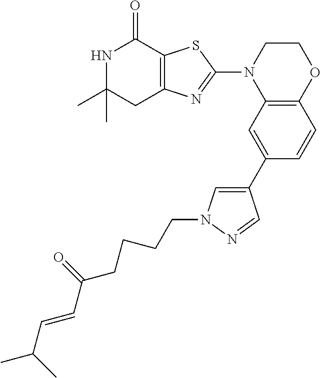
VI-6 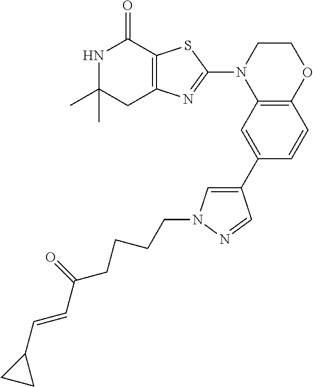
VI-7 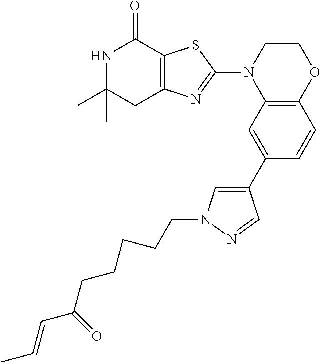
VI-8 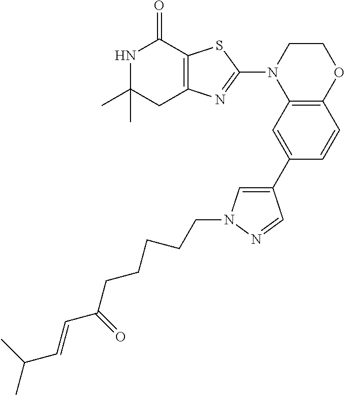
VI-9 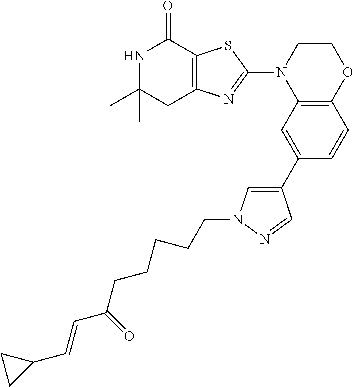
VI-10 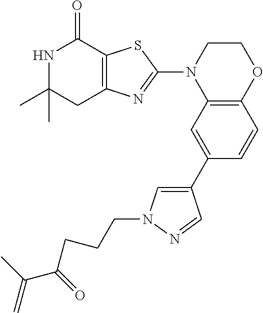
VI-11 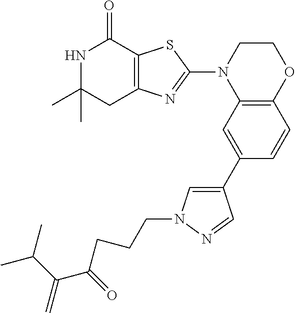
VI-12 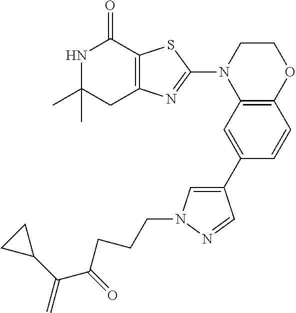
VI-13 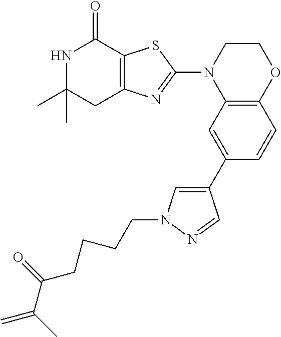
VI-14 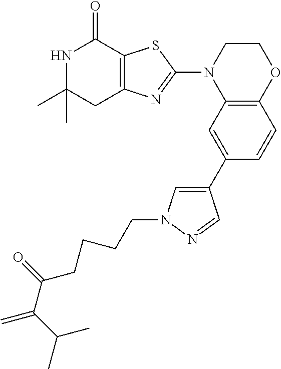
VI-15 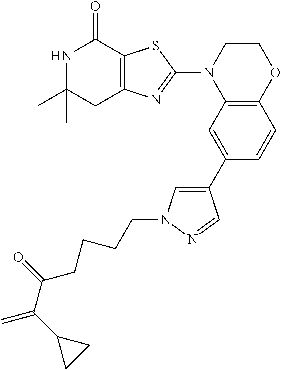
VI-16 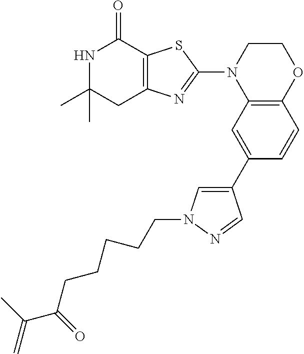
VI-17 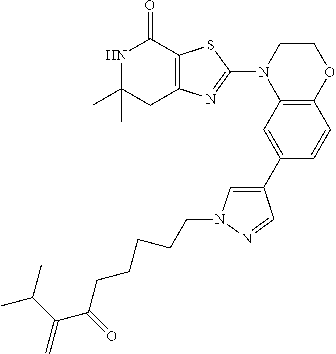
VI-18 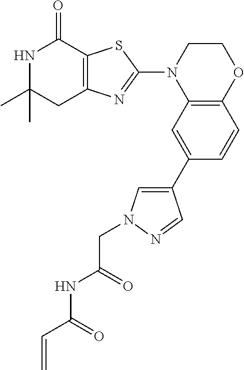
VI-19 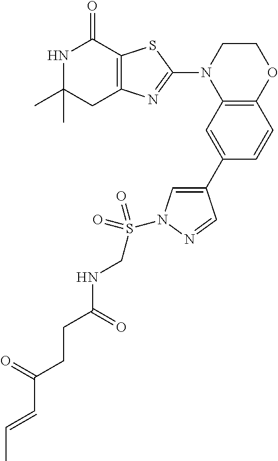
VI-20 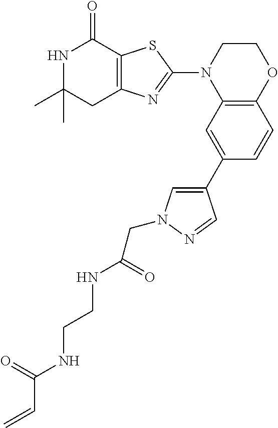
VI-21 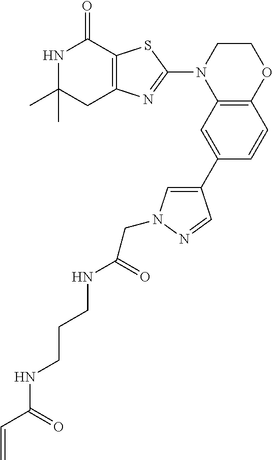
VI-22 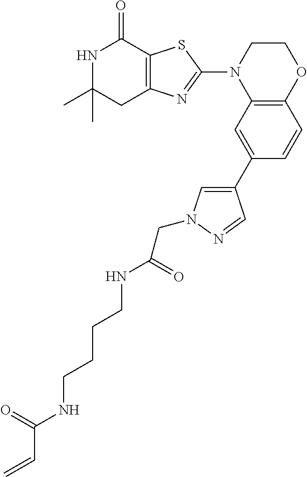
VI-23 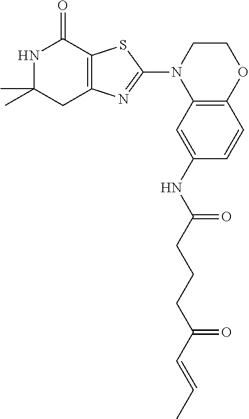
VI-24 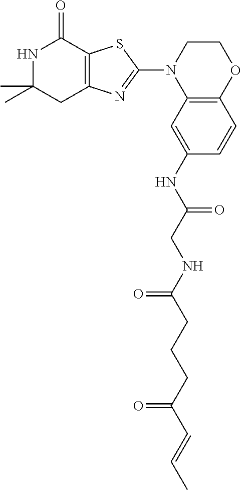
VI-25

In certain embodiments, the present invention provides any compound selected from those depicted in Table 11, above, or a pharmaceutically acceptable salt thereof.

Exemplary compounds of formula VII are set forth in Table 12, below:

TABLE 12
Exemplary Compounds of Formula VII

VII-1

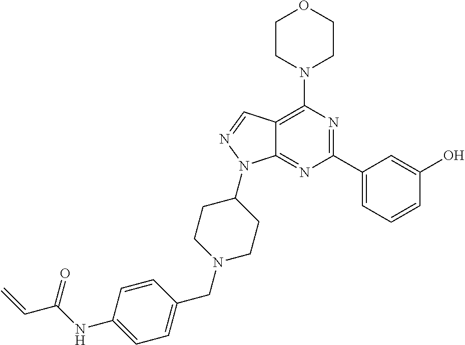

VII-2

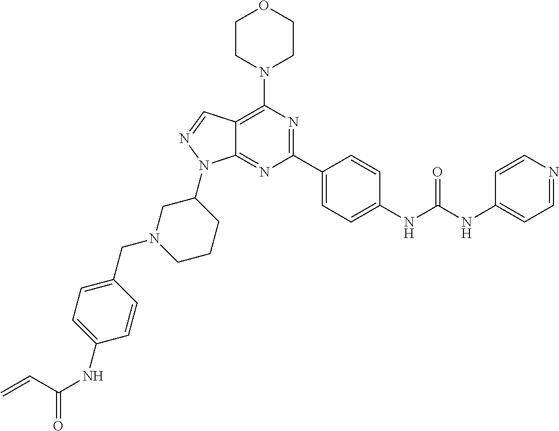

VII-3

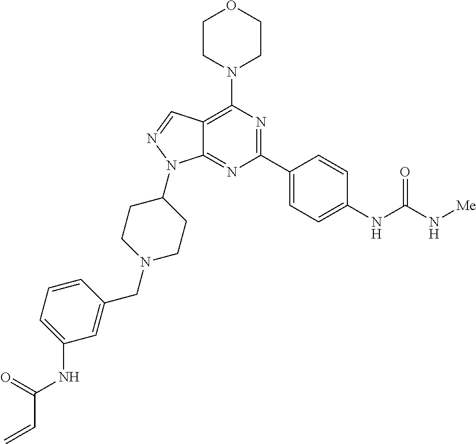

VII-4

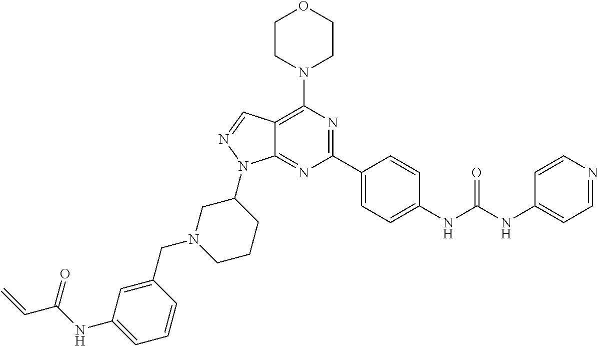

VII-5

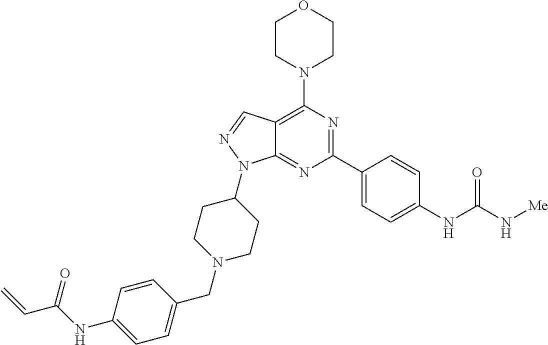

VII-6

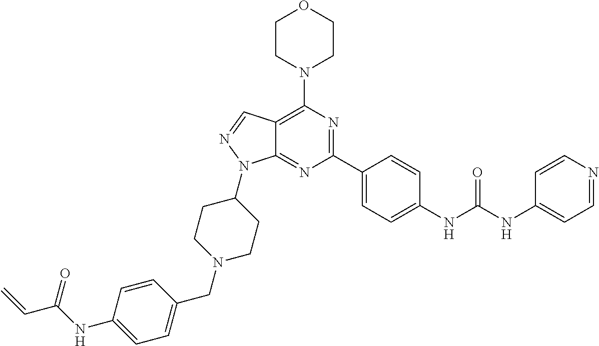

VII-7

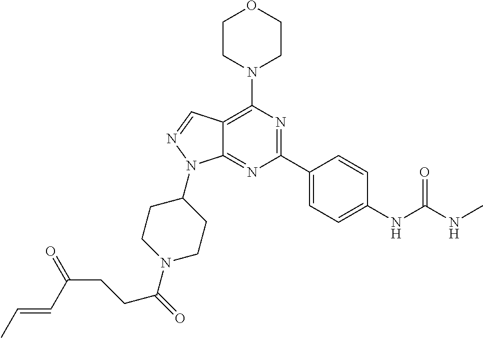

VII-8

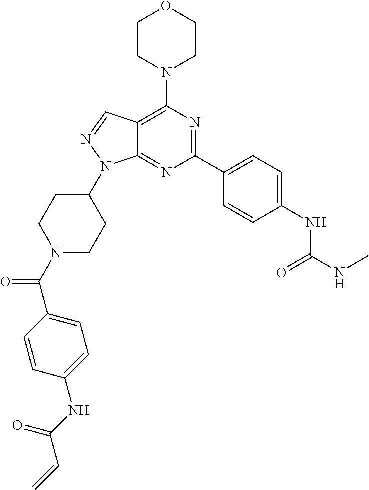

VII-9

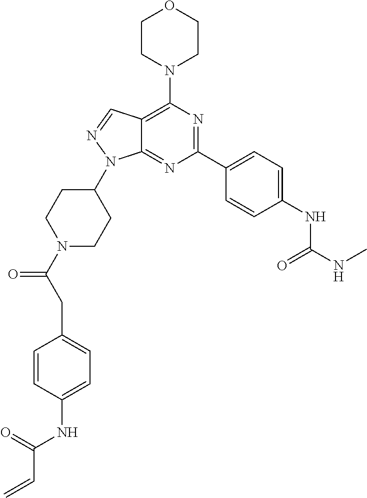

VII-10

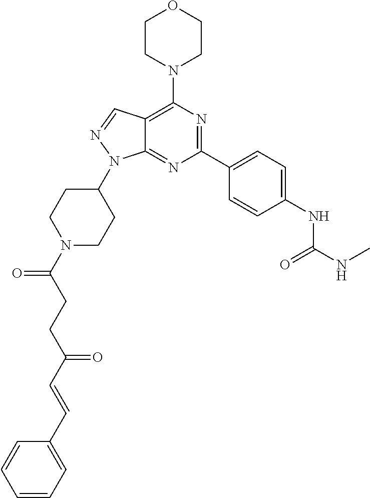

VII-11

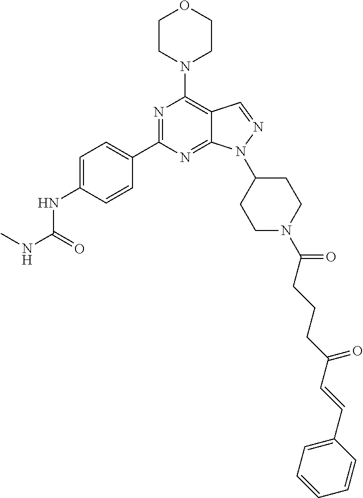

VII-12

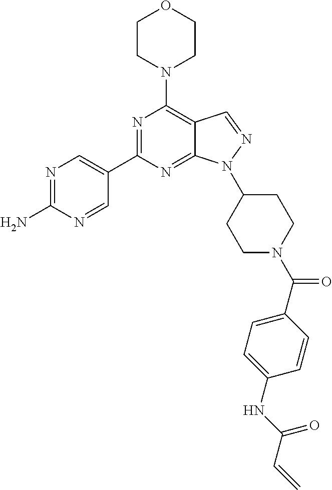

VII-13

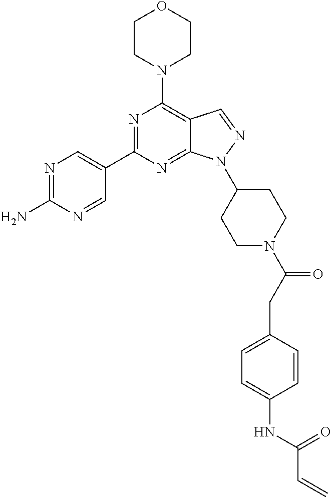

In certain embodiments, the present invention provides any compound selected from those depicted in Table 12, above, or a pharmaceutically acceptable salt thereof.

Exemplary compounds of formula VIII are set forth in Table 13, below:

TABLE 13
Exemplary Compounds of Formula VIII

VIII-1

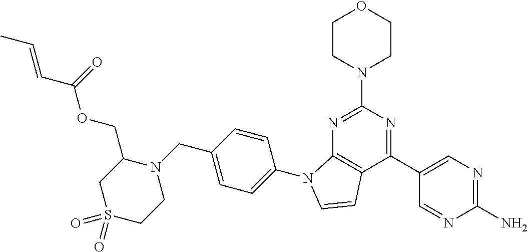

VIII-2

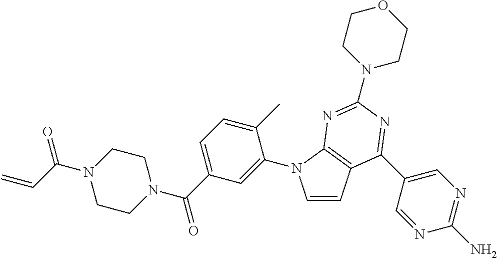

VIII-3

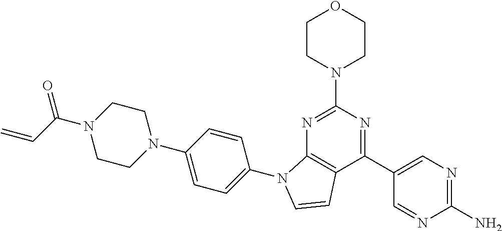

VIII-4

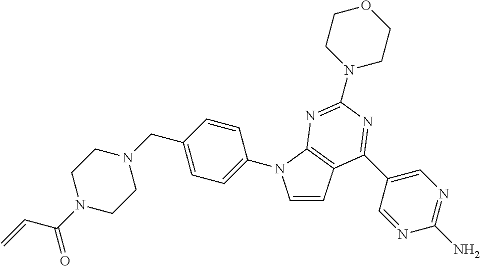

VIII-5

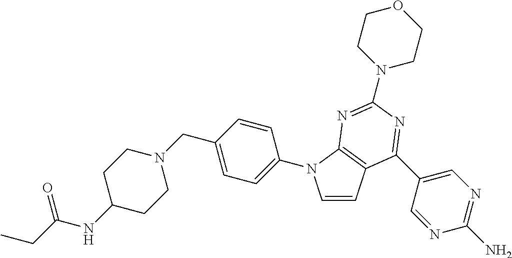

VIII-6

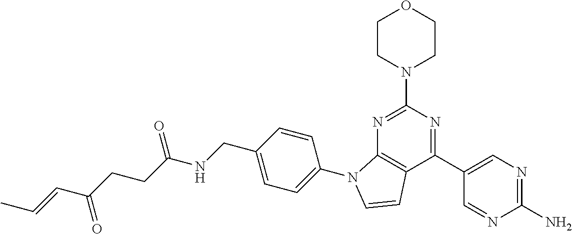

VIII-7

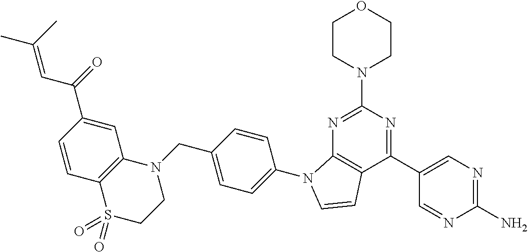

In certain embodiments, the present invention provides any compound selected from those depicted in Table 13, above, or a pharmaceutically acceptable salt thereof.

Exemplary compounds of formula IX are set forth in Table 14, below:

TABLE 14
Exemplary Compounds of Formula IX

IX-1

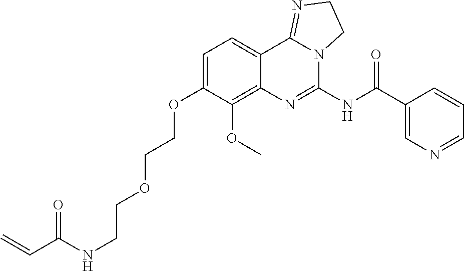

IX-2

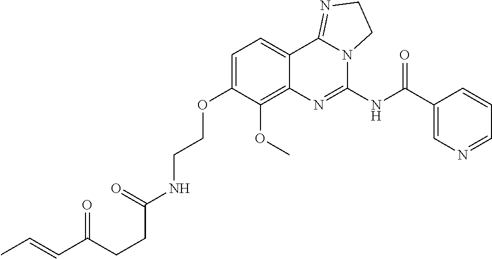

IX-3

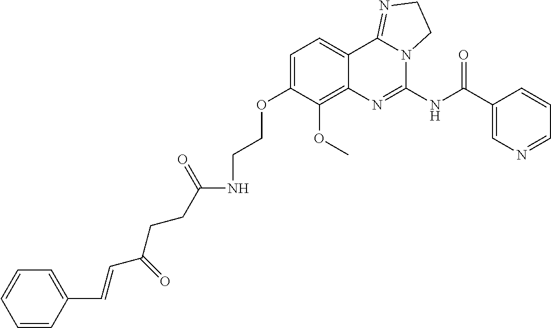

IX-4

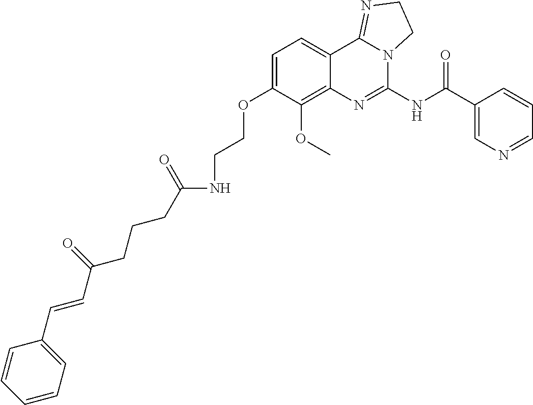

IX-5

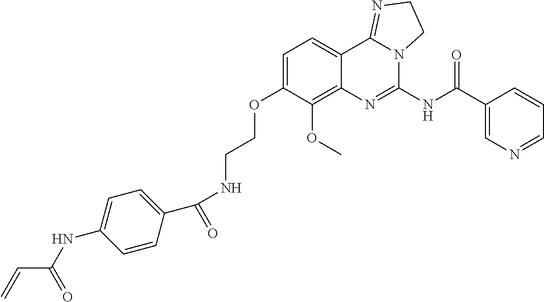

IX-6

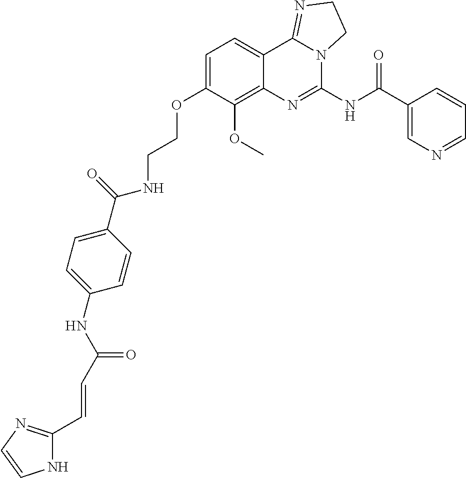

In certain embodiments, the present invention provides any compound selected from those depicted in Table 14, above, or a pharmaceutically acceptable salt thereof.

Exemplary compounds of formula X are set forth in Table 15, below:

TABLE 15
Exemplary Compounds of Formula X

X-1

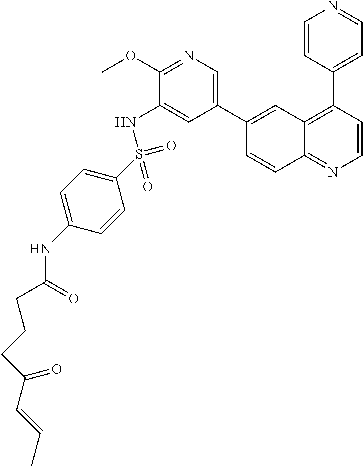

In certain embodiments, the present invention provides any compound selected from those depicted in Table 15, above, or a pharmaceutically acceptable salt thereof.

Exemplary compounds of formula XI are set forth in Table 16, below:

TABLE 16
Exemplary Compounds of Formula XI

XI-1

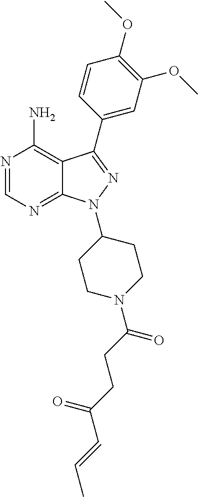

XI-2

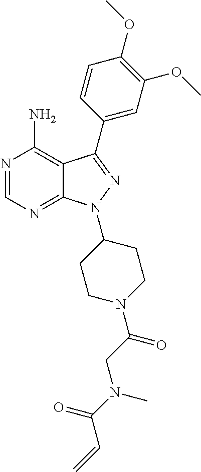

XI-3

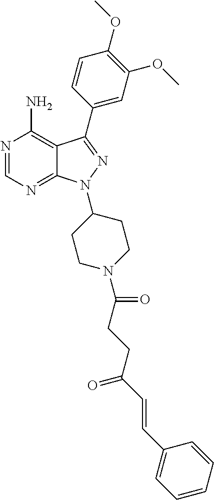

XI-4

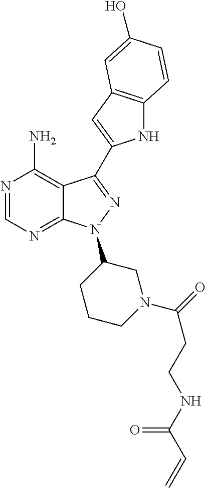

XI-5

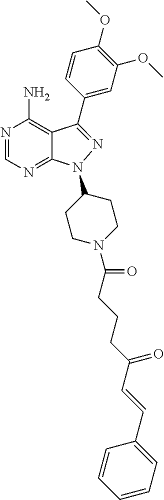

XI-6

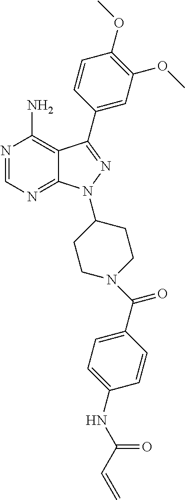

XI-7

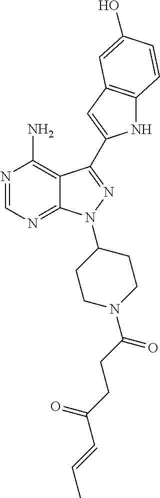

XI-8

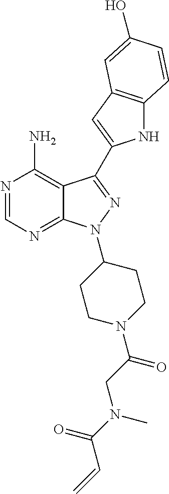

In certain embodiments, the present invention provides any compound selected from those depicted in Table 16, above, or a pharmaceutically acceptable salt thereof.

Exemplary compounds of formula XII are set forth in Table 17, below:

TABLE 17
Exemplary Compounds of Formula XII

XII-1

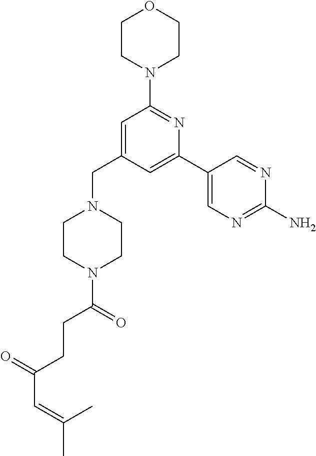

XII-2

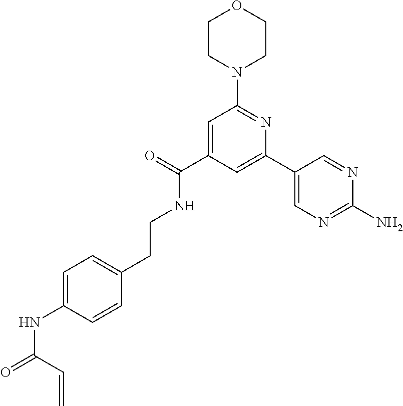

XII-3

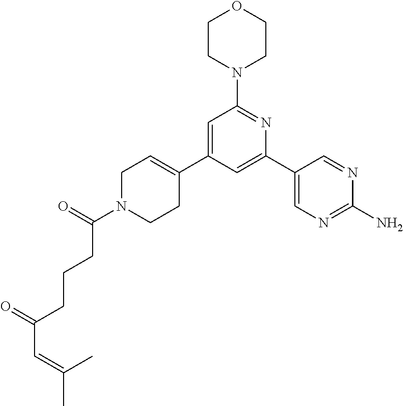

XII-4

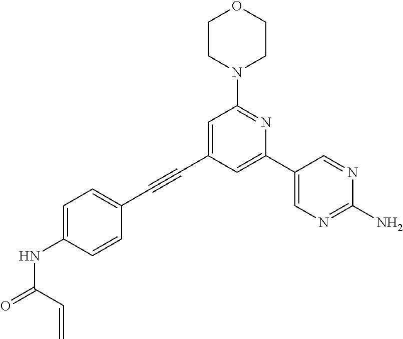

XII-5

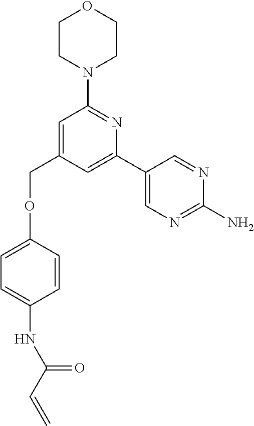

XII-6

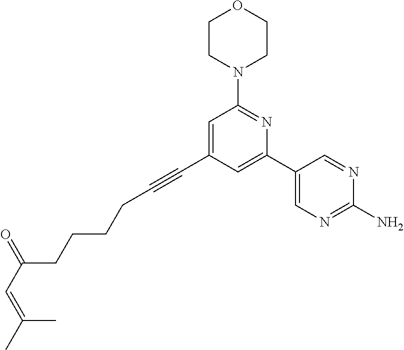

XII-7

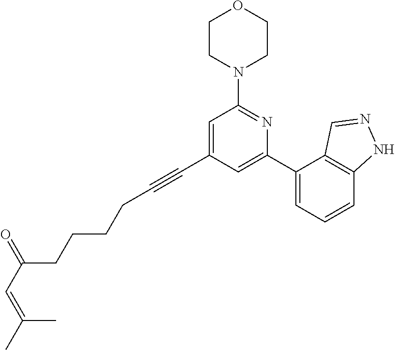

XII-8

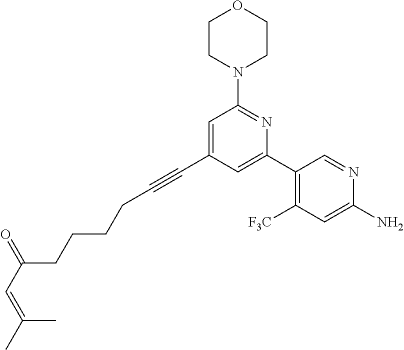

XII-9

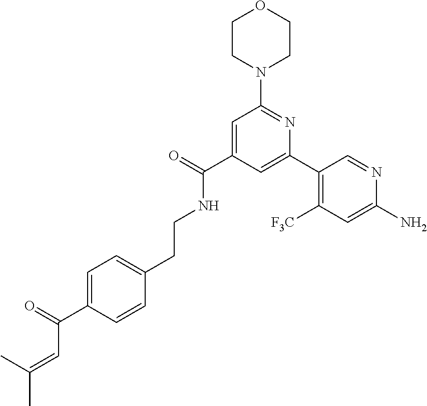

XII-10

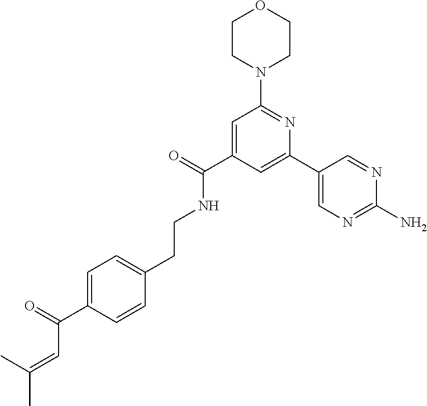

XII-11

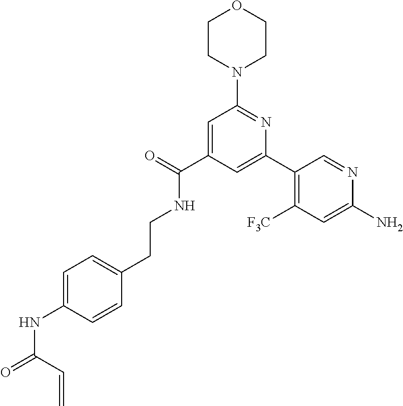

XII-12

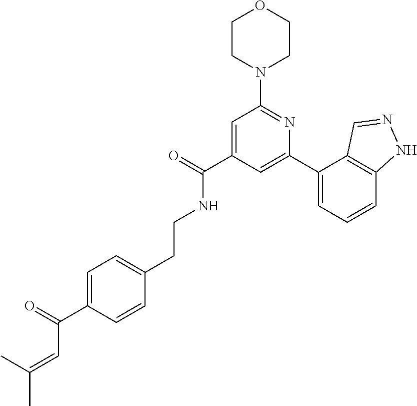

XII-13

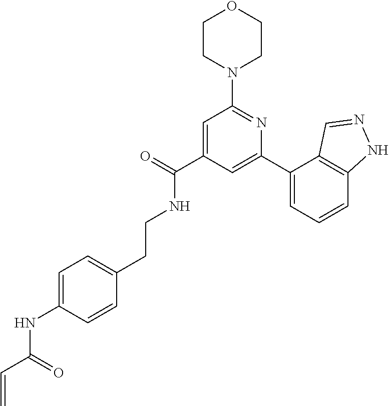

XII-14

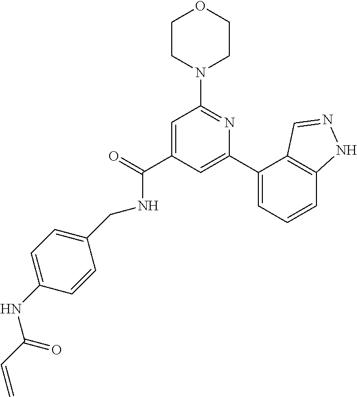

XII-15

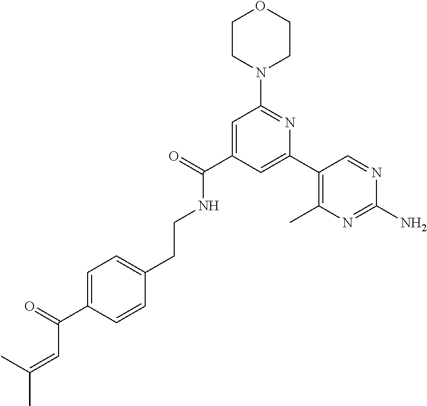

XII-16

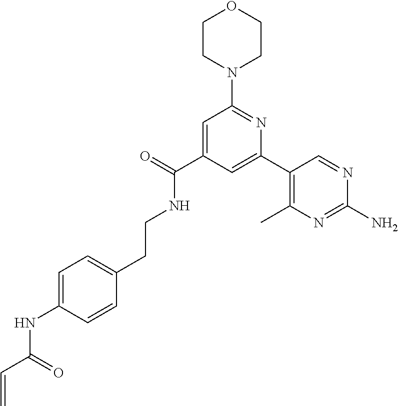

XII-17

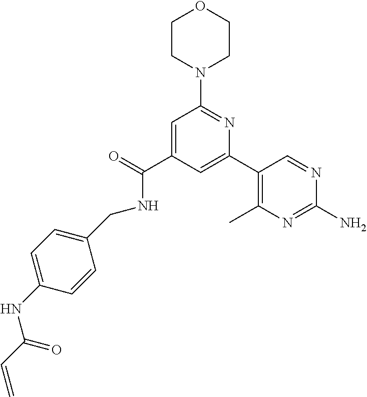

XII-18

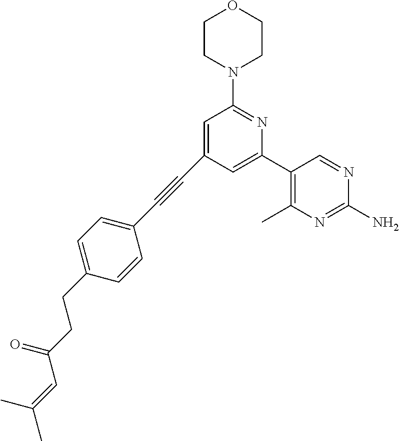

XII-19

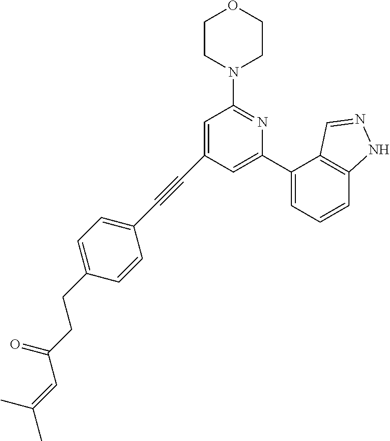

XII-20

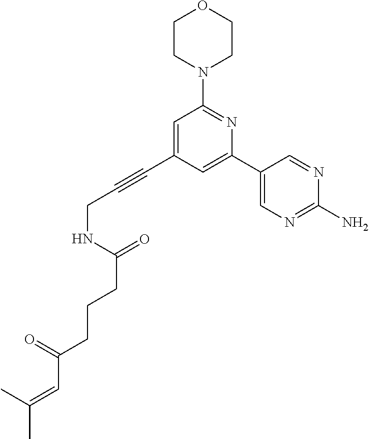

XII-21

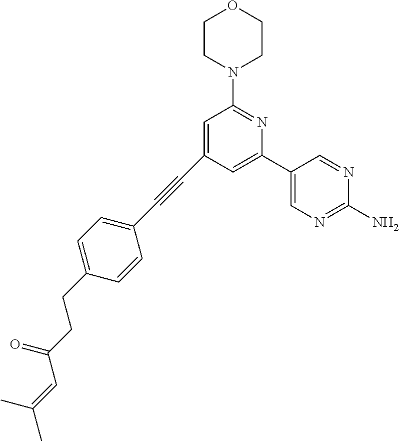

XII-22

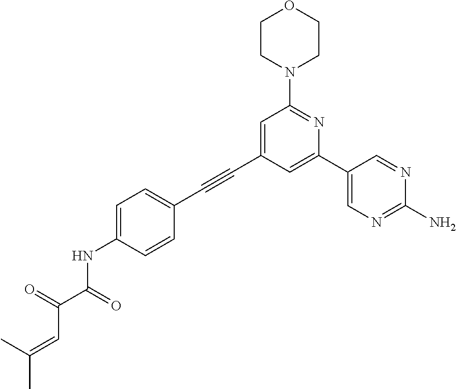

XII-23

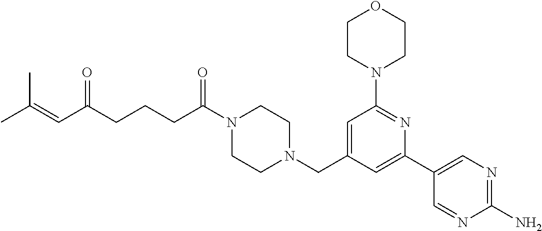

XII-24

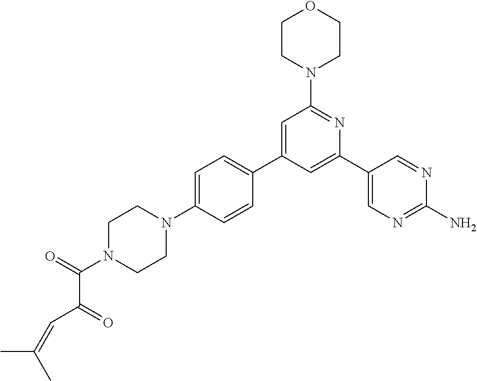

XII-25

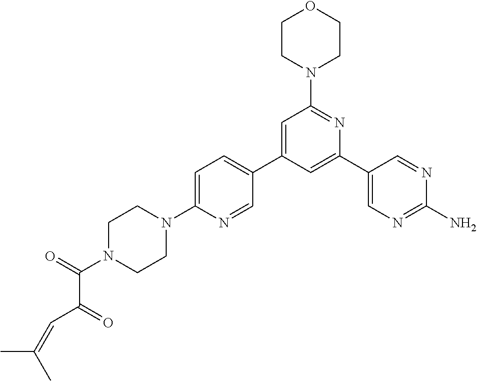

XII-26

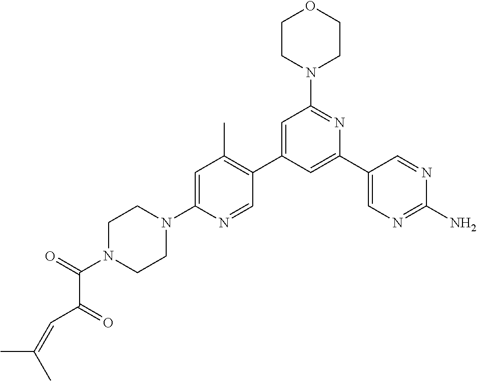

XII-27

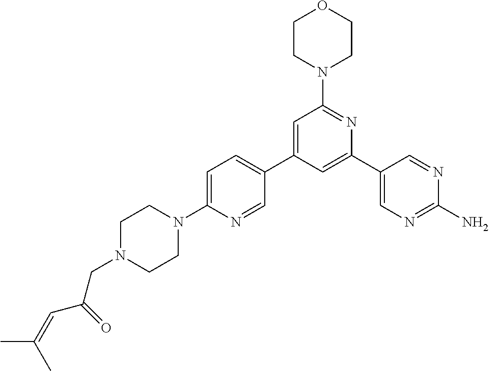

XII-28

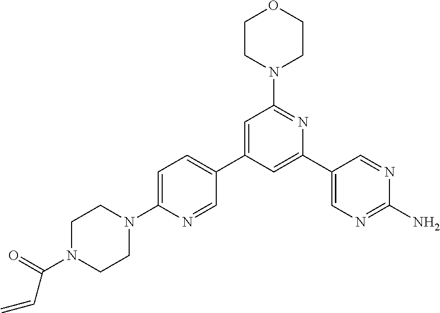

XII-29

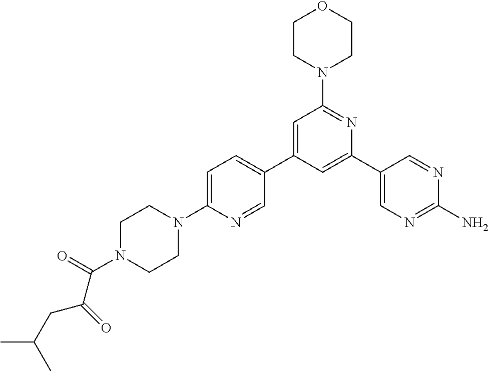

XII-30

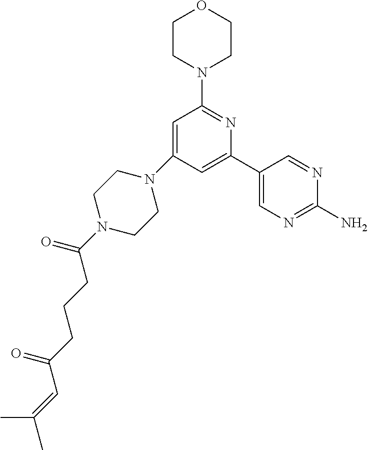

XII-31

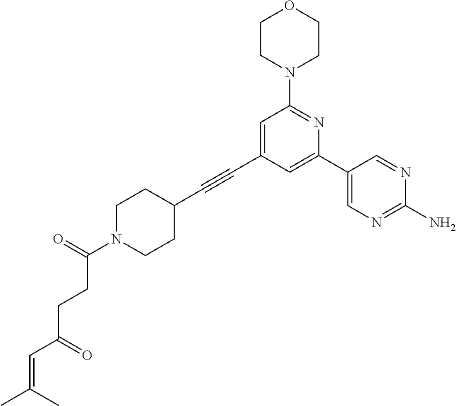

XII-32

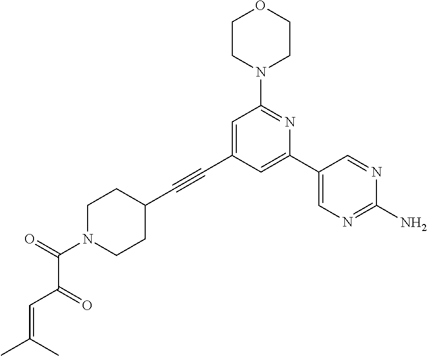

XII-33

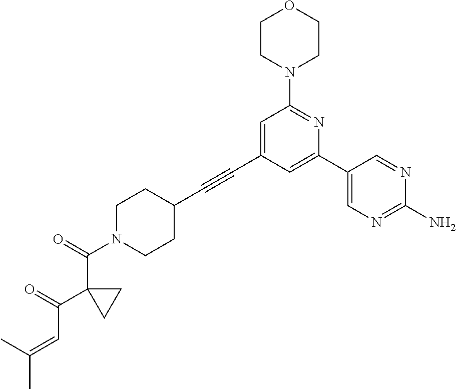

XII-34

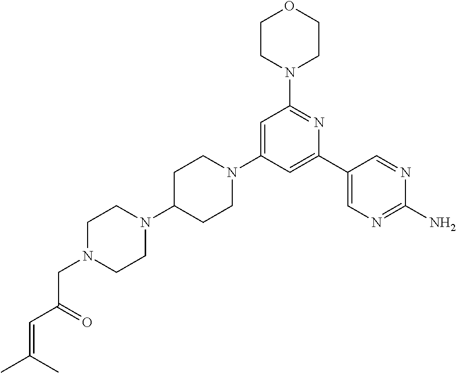

XII-35

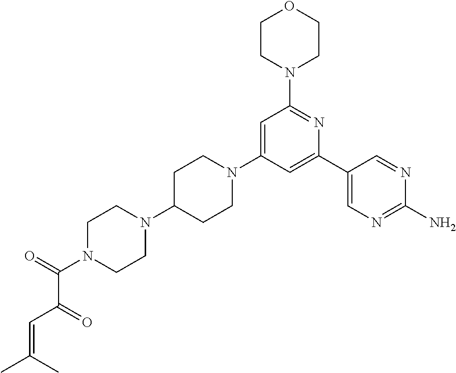

XII-36

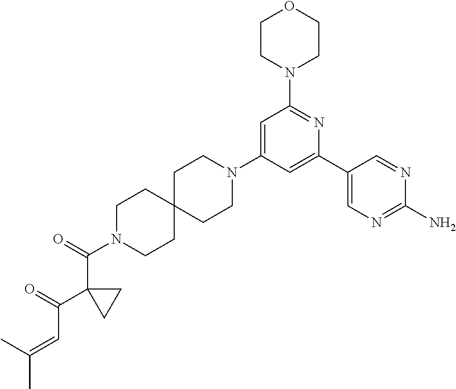

XII-37

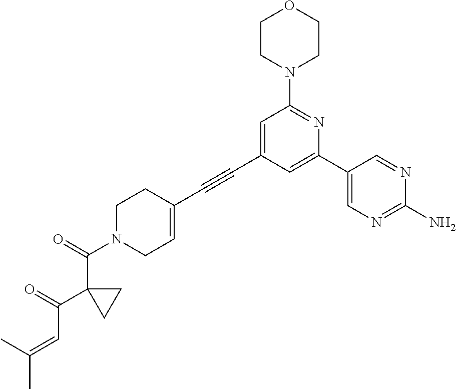

XII-38

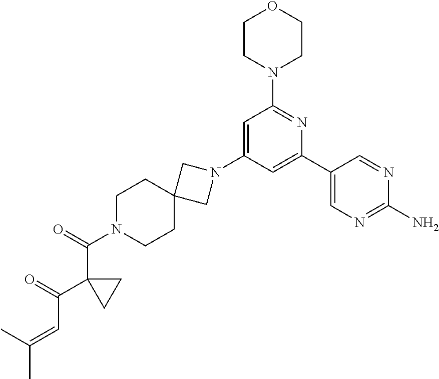

XII-39

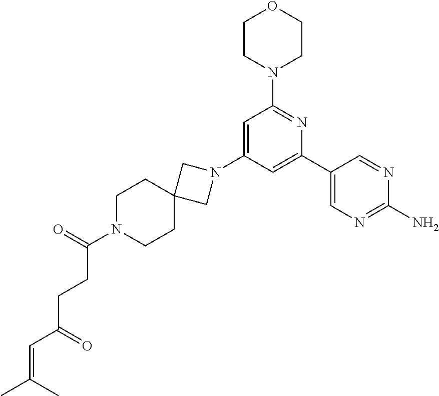

XII-40

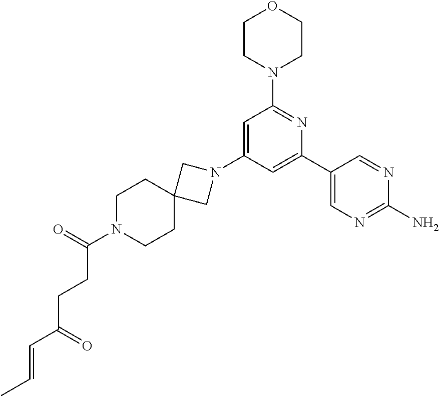

XII-41

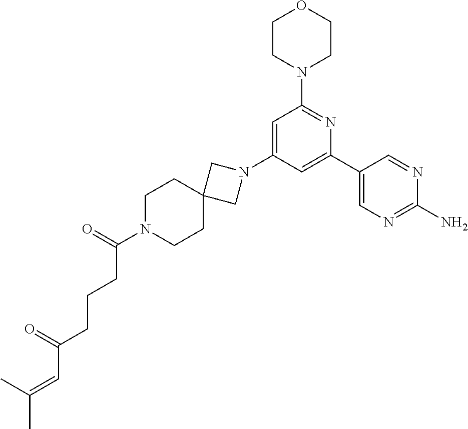

XII-42

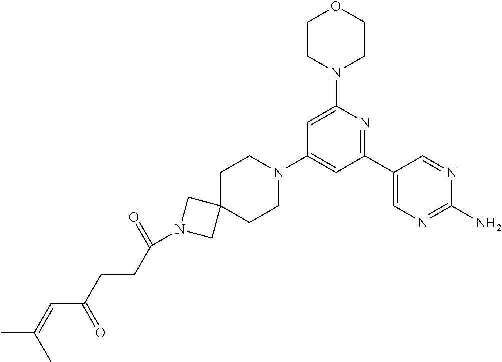

XII-43

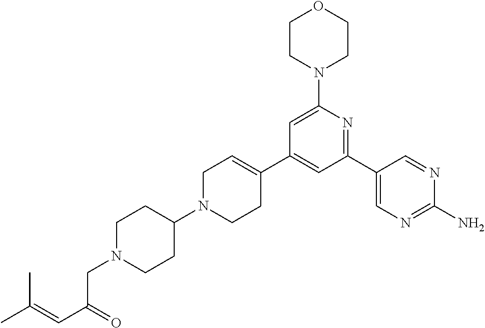

XII-44

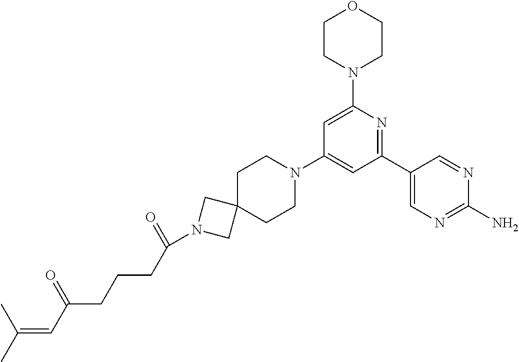

XII-45

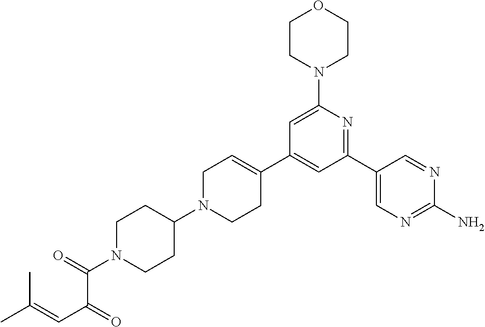

XII-46

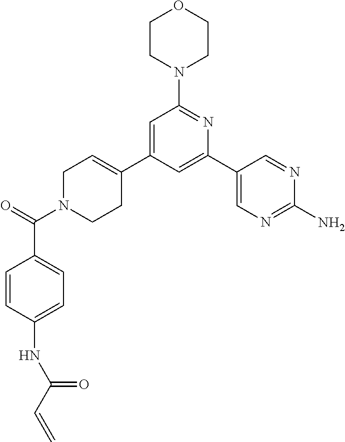

XII-47

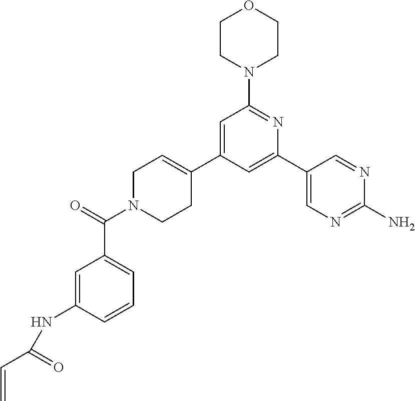

XII-48

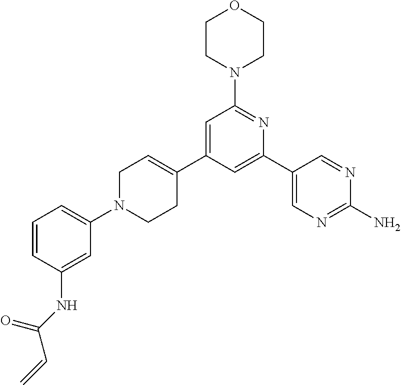

XII-49

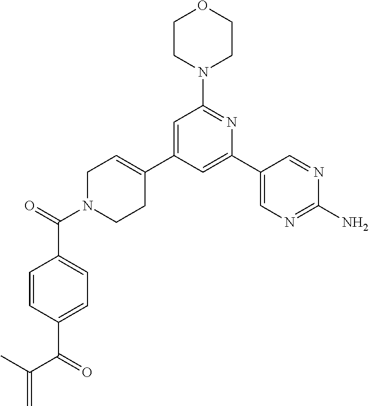

XII-50

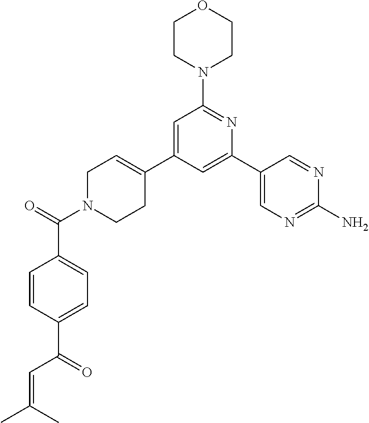

XII-51

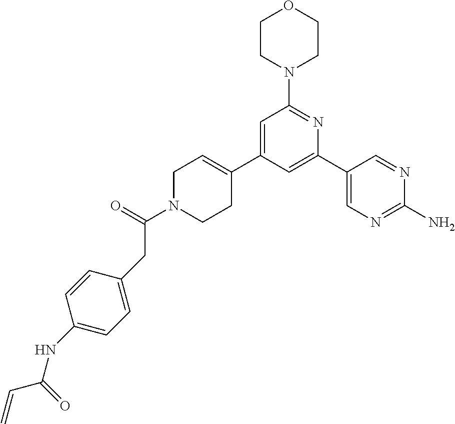

XII-52

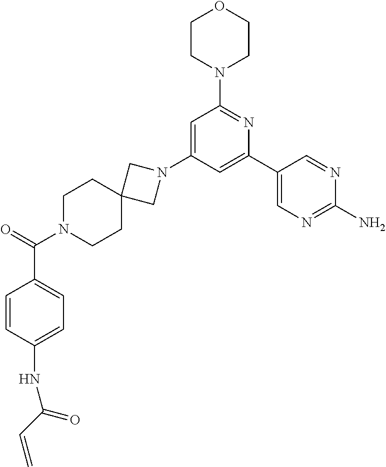

XII-53

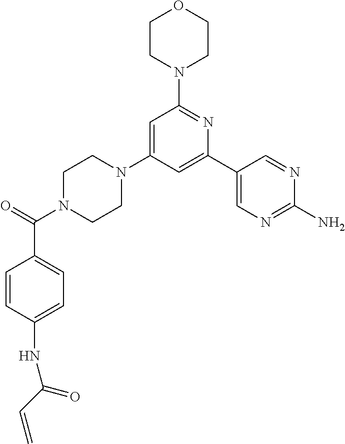

XII-54

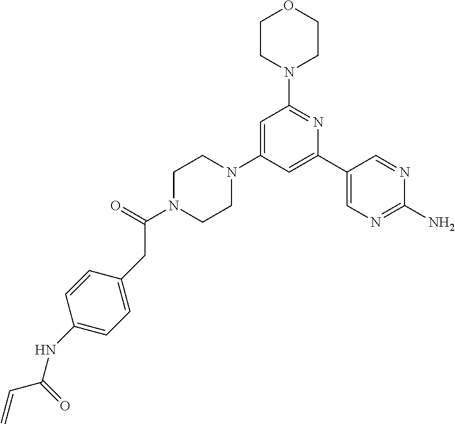

In certain embodiments, the present invention provides any compound selected from those depicted in Table 17, above, or a pharmaceutically acceptable salt thereof.

### General Methods of Making Provided Compounds

In certain embodiments, the provided compounds of formula I are generally prepared according to Scheme 1.

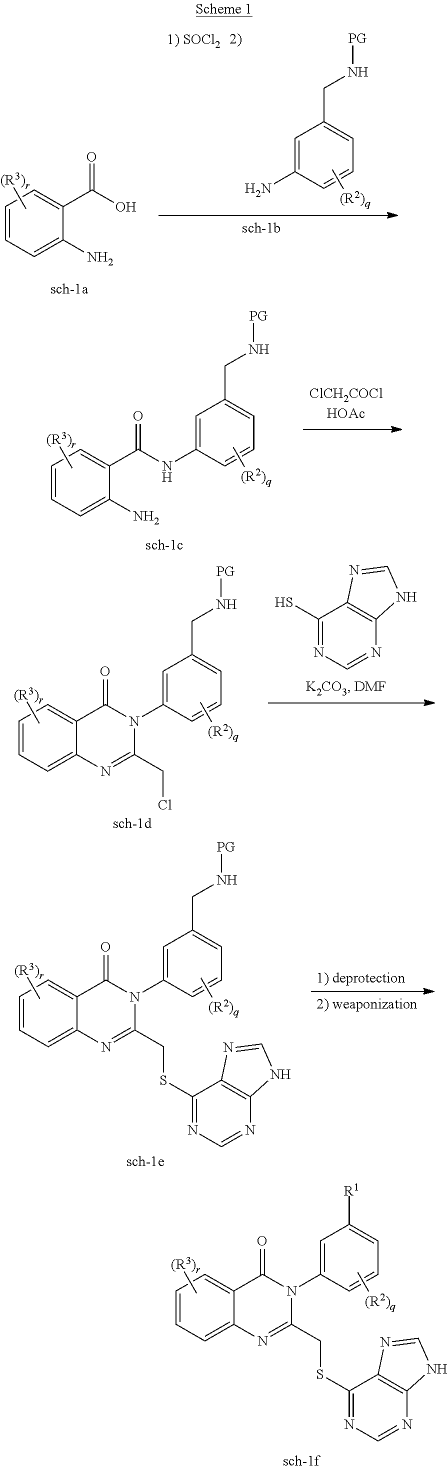

wherein PG is an amino protection group and each variable is as defined and described herein.

A substituted 2-aminobenzoic acid (sch-1a) is converted to its acid chloride by treatment of thionyl chloride at elevated temperature (40-100° C.). The intermediate is then reacted with excess amount of aniline sch-1b in CHCl₃ under reflux to give compound sch-1c. Upon treatment with chloroacetyl chloride in acetic acid under reflux, compound sch-1d can be obtained. Intermediate sch-1d then can react with mercaptopurine at the presence of a base (i.e K₂CO₃) to form sch-1e. The protection group is then removed and a war head group can be introduced to give compound sch-1f.

In certain embodiments, provided compounds of formula II-a are generally prepared according to Scheme 2.

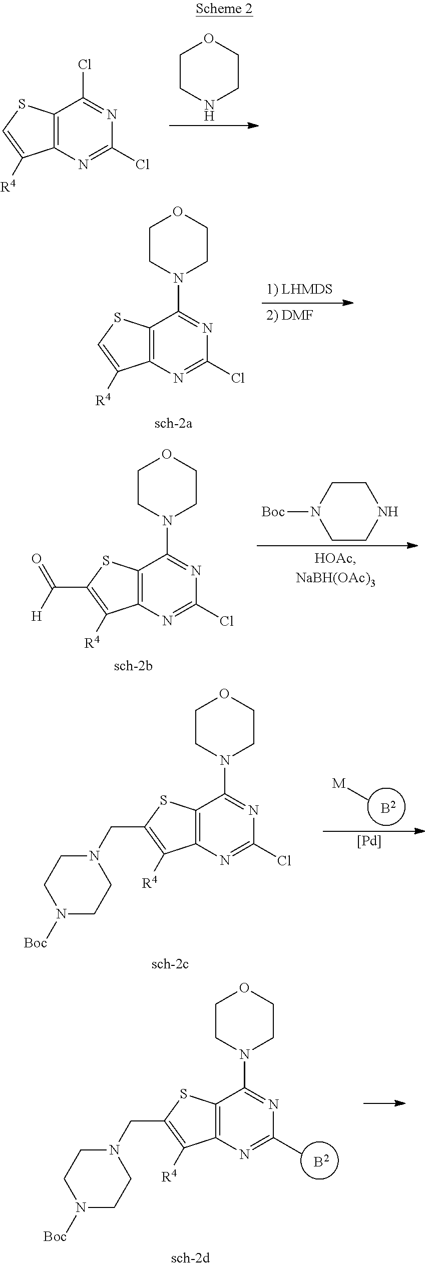

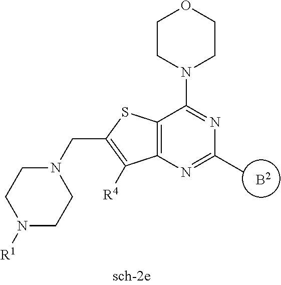

wherein M is a boronic acid or stannyl group.

Compound sch-2a is prepared by reacting morpholine with substituted 2,4-dichlorothieno[3,2-d]pyrimidine in methanol at RT. A formyl group can be introduced upon treatment of sch-2a with butyl lithium at low temperature and followed by the addition of DMF. Reductive amination of sch-2b with tert-butyl piperazine-1-carboxylate produces sch-2c. A palladium catalyzed coupling of sch-2c with a boronic acid or a stannyl compound gives compound sch-2d. The boc group is then removed and a war head group can be introduced to give compound sch-2e.

In another embodiment, compounds of formula II-a can be prepared as described in Scheme 3.

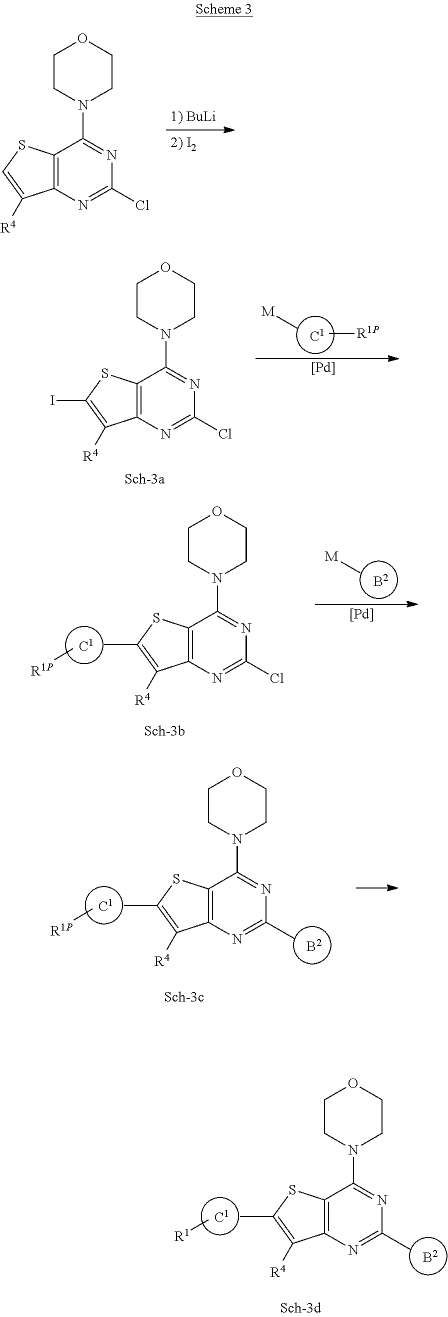

wherein M is a boronic acid or stannyl group, and R^{1P} is a precursor to R¹.

Intermediate sch-3a is prepared by de-protonation of substituted 4-(2-chlorothieno[3,2-d]pyrimidin-4-yl)morpholine with n-BuLi at low temperature followed by treatment with iodine. A palladium catalyzed selective coupling of sch-3a with a boronic acid or a stannyl compound gives compound sch-3b. The second palladium catalyzed coupling with another boronic acid or stannyl compound at higher temperature gives compound sch-3c. In the last step, the R^{1P} group is converted to a warhead group R¹ as shown in sch-3d.

In certain embodiments, provided compounds of formula II-c are generally prepared according to Scheme 4.

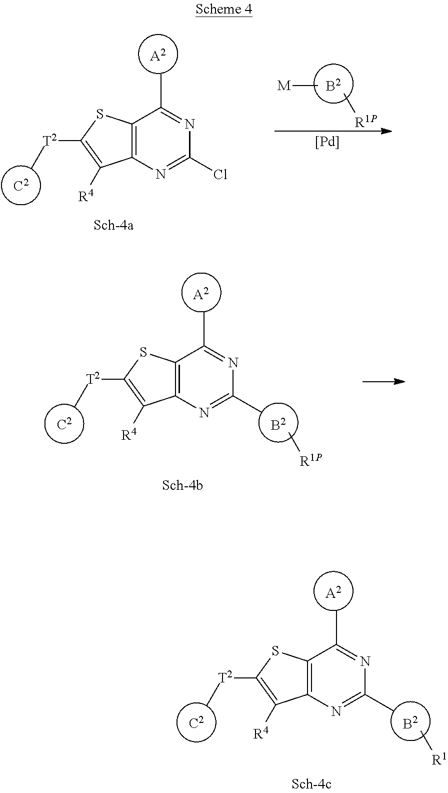

wherein M is a boronic acid or stannyl group, and R^{1P} is a precursor to R¹.

Compound sch-4a is prepared according to scheme 2 and scheme 3. A palladium catalyzed coupling of sch-4a with a boronic acid or a stannyl compound gives compound sch-4b. The R^{1P} group is then converted to a war head group R¹ in the last step to give sch-4c.

In certain embodiments, provided compounds of formula III or IV are generally prepared according to Scheme 5.

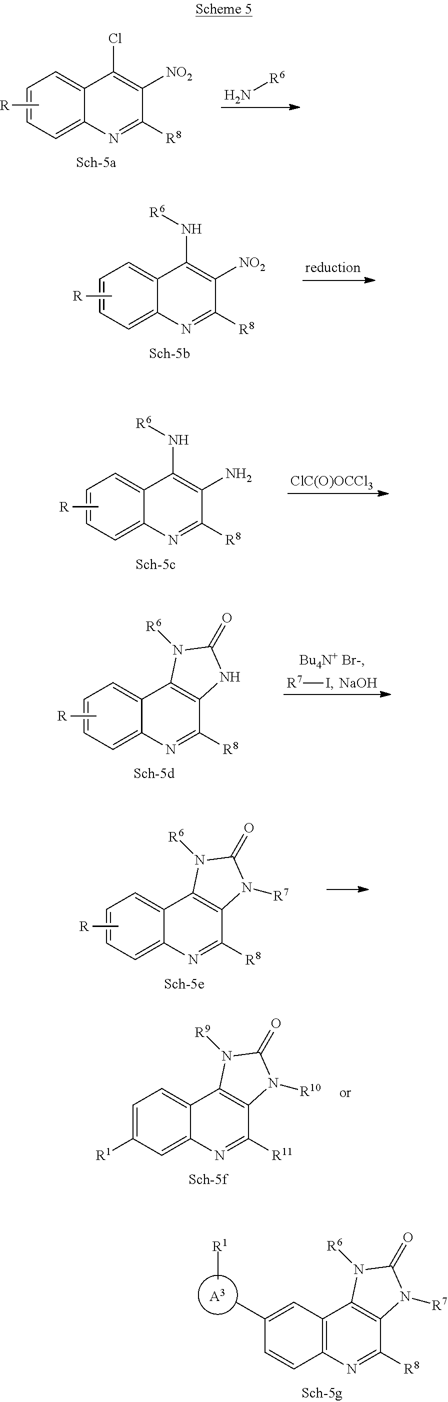

Compound Sch-5a, which bears an R group suitable to convert to a war head group R1 in a later step, is reacted with an amine to form compound sch-5b. The nitro group is then reduced by a reducing agent (i.e. hydrogenation) provides compound sch-5c, which forms a cyclic urea sch-5d upon treatment with phosgene or ClC(O)OCCl₃. The urea is alkylated by an alkyliodide under the phase transferring condition to form compound sch-5e. In the last step the R group is converted to a WH group R¹ to give either sch-5f or sch-5g.

In certain embodiments, provided compounds of formula V-a or V-b are generally prepared according to Scheme 6.

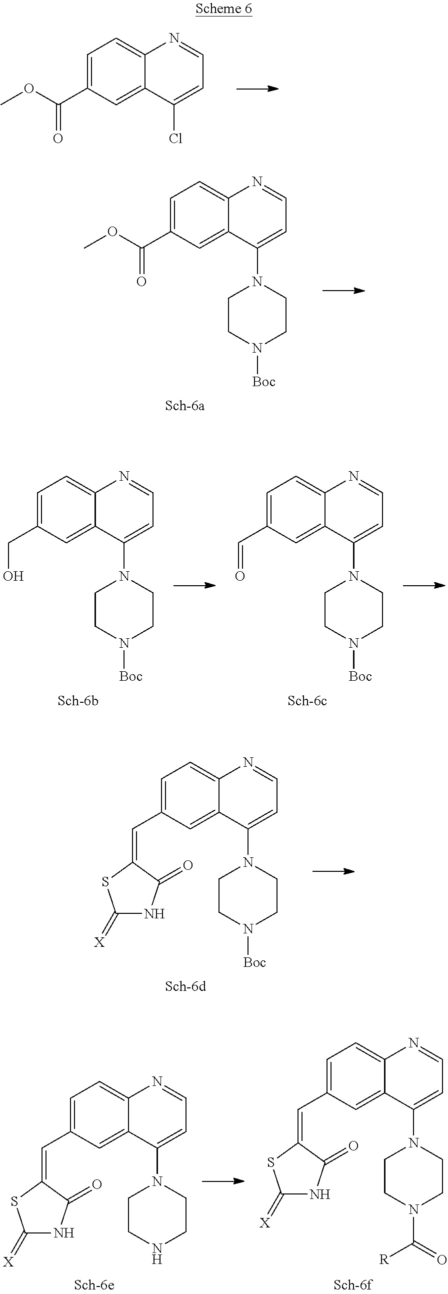

Compound sch-6a is prepared by the addition of a mono-protected piperazine to the methyl 4-chloroquinoline-6-carboxylate. The reduction of sch-6a with a metal-hydride reagent such as lithium aluminum hydride provides compound sch-6b, which can be oxidized with an oxidant such as Dess-Martin periodinate to yield compound sch-6c. Condensation of sch-7c with thiazolidine-2,4-dione or 2-(2,6-dichlorophenylamino)thiazol-4(5H)-one in the presence of a base such as piperidine gives the alkene sch-6d. Deprotection of sch-6d with an acid such as HCl yields sch-6e. In the last step, a war head group R can be connected using an amino acid coupling to give compound sch-6f.

In certain embodiments, provided compounds of formula VI-a are generally prepared according to Scheme 7.

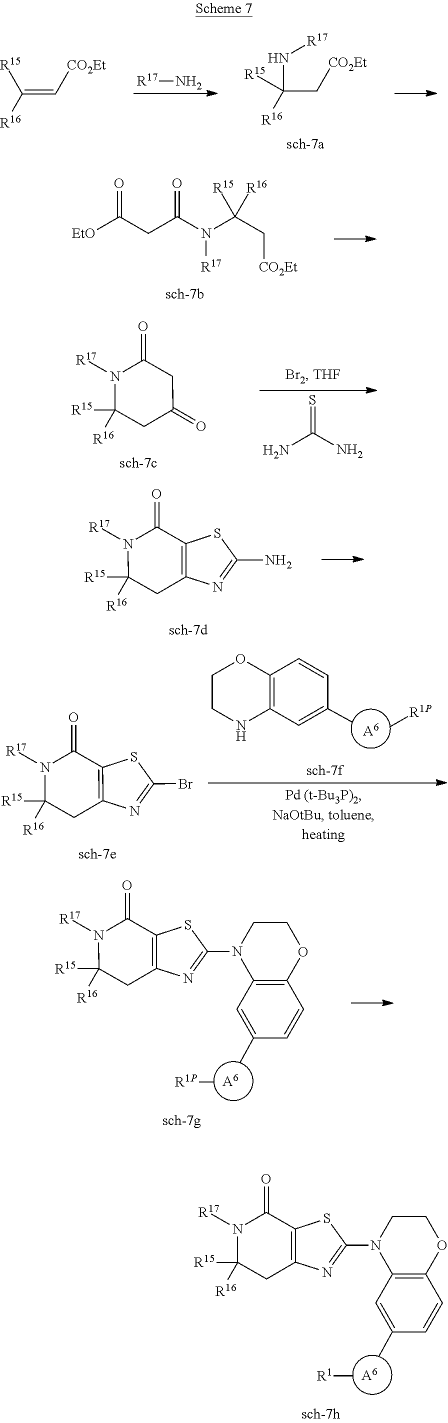

wherein R^{1P} is a precursor to R¹.

Compound Sch-7a is prepared by the addition of an amine to the substituted acrylate. The treatment of sch-7a with ethyl malanoyl chloride at the presence of a base (i.e. TEA) gives compound sch-7b, which cyclize upon base treatment and forms compound sch-7c after decarboxylation. Compound sch-7c is then treated with bromine followed by addition of thiourea and DIPEA to give the aminothiazole sch-7d. The amino group is then converted to a bromide by reacting with n-butyl nitrite and CuBr₂. The resulting bromothiazole sch-7e is coupled with 3,4-dihydro-2H-benzo[b][1,4]oxazine (sch-7f) under the Buchwald condition to give compound sch-7g. In the last step, R^{1P} group is then converted to a war head group R¹ to give compound sch-7h.

In certain embodiments, provided compounds of formula VII are generally prepared according to Scheme 8.

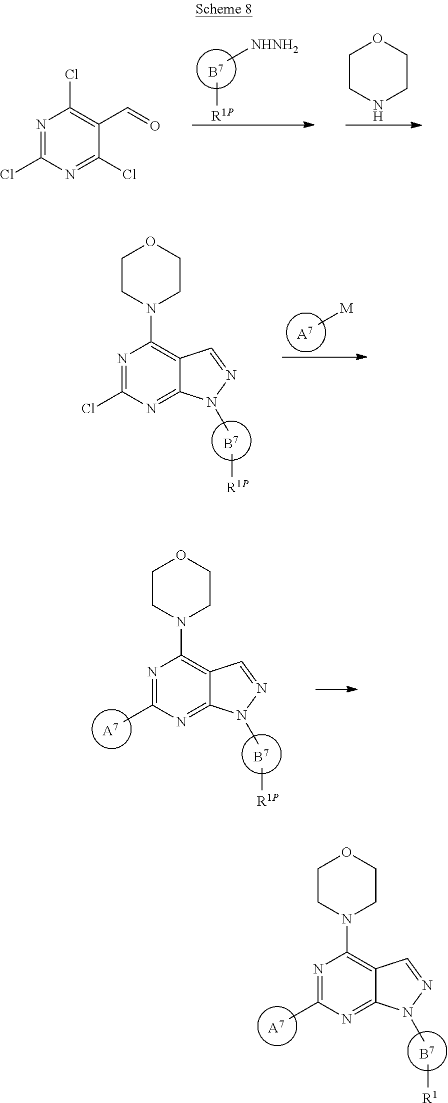

wherein M is a boronic acid or stannyl group, and R^{1P} is a precursor to R¹.

Compound sch-8a is prepared by the addition of a hydrazine to 2,4,6-trichloropyrimidine-5-carbaldehyde, followed by displacement of a chloro group by morpholine. Treatment of sch-8a with an arylboronate or stannane results in compound sch-8b. In the last step, R^{1P} group is then converted to a war head group R¹ to give compound sch-8c.

In certain embodiments, provided compounds of formula IX are generally prepared according to Scheme 9.

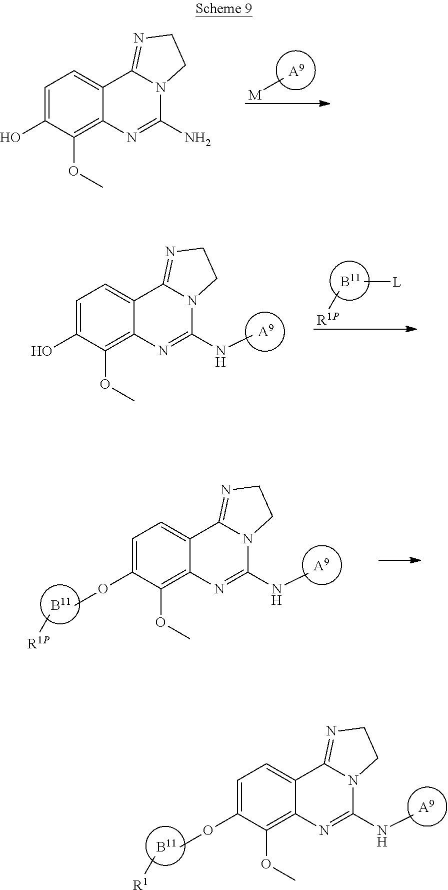

wherein M is an acid, acyl chloride, sulfonyl chloride, isocyanate, etc., L is a leaving group (such as halide, mesylate, tosylate), and R^{1P} is a precursor to R¹.

Compound sch-9a is prepared by coupling an aryl group to an amino group. Displacement of a leaving group with the phenol of compound sch-9a results in compound sch-9b. In the last step, R^{1P} group is then converted to a war head group R¹ to give compound sch-9c.

In certain embodiments, provided compounds of formula XI are generally prepared according to Scheme 10.

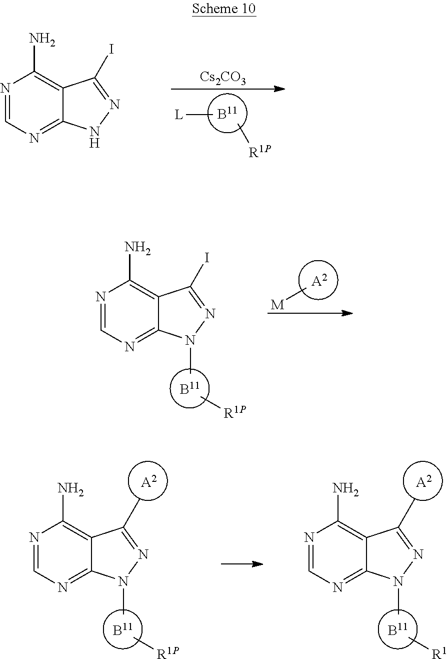

wherein M is a boronic acid or stannyl group, L is a leaving group (such as mesylate or tosylate), and R^{1P} is a precursor to R¹.

Compound sch-10a is prepared by coupling a B¹¹ group to the pyrazolopyrimidine scaffold. Suzuki or Stille coupling gives compound sch-10b. In the last step, R^{1P} group is then converted to a war head group R¹ to give compound sch-10c.

In certain embodiments, provided compounds of formula XII are generally prepared according to Scheme 11.

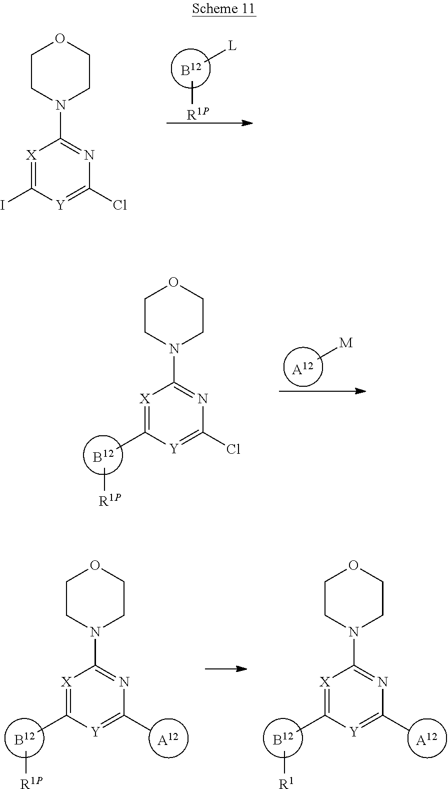

wherein X and Y are independently N or CH, M is a boronic acid or stannyl group, L is a boronic acid or stannyl group, and R^{1P} is a precursor to R¹.

A first Suzuki or Stille coupling affords compound sch-11a, and a second Suzuki or Stille coupling affords compound sch-11b. In the last step, R^{1P} group is then converted to a war head group R¹ to give compound sch-11c.

### 4. Uses, Formulation and Administration

Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably inhibit a PI3 kinase, or a mutant thereof (for example, Glu542, Glu545 and His1047), in a biological sample or in a patient. In certain embodiments, the amount of compound in compositions of this invention is such that is effective to measurably inhibit a PI3 kinase, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

As used herein, the term “inhibitorily active metabolite or residue thereof” means that a metabolite or residue thereof is also an inhibitor of a PI3 kinase, or a mutant thereof (for example, Glu542, Glu545 and His1047).

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Uses of Compounds and Pharmaceutically Acceptable Compositions

Compounds and compositions described herein are generally useful for the inhibition of kinase activity of one or more enzymes.

Examples of kinases that are inhibited by the compounds and compositions described herein and against which the methods described herein are useful include PI3Kα, PI3Kγ, PI3Kδ, PI3Kβ Class 1A (PI3Kβ), PI3Kβ Class 2 (PI3KC2β), mTOR, DNA-PK, ATM kinase and/or PI4KIIIα, or a mutant thereof.

The activity of a compound utilized in this invention as an inhibitor of PI3Kα, PI3Kγ, PI3δ, PI3Kβ, PI3KC2β, mTOR, DNA-PK, ATM kinase and/or PI4KIIIα, or a mutant thereof, may be assayed in vitro, in vivo or in a cell line. In vitro assays include assays that determine inhibition of either the phosphorylation activity and/or the subsequent functional consequences, or ATPase activity of activated PI3Kα, PI3Kβ, PI3Kδ, PI3Kβ, PI3KC2β, mTOR, DNA-PK, ATM kinase and/or PI4KIIIα, or a mutant thereof. Alternate in vitro assays quantitate the ability of the inhibitor to bind to PI3Kα, PI3Kγ, PI3Kδ, PI3Kβ, PI3KC2β, mTOR, DNA-PK, ATM kinase and/or PI4KIIIα. Inhibitor binding may be measured by radiolabeling the inhibitor prior to binding, isolating the inhibitor/PI3Kα, inhibitor/PI3Kγ, inhibitor/PI3Kδ, inhibitor/PI3Kβ, inhibitor/PI3KC2β, inhibitor/mTOR, inhibitor/DNA-PK, inhibitor/ATM kinase or inhibitor/PI4KIIIα complex and determining the amount of radiolabel bound. Alternatively, inhibitor binding may be determined by running a competition experiment where new inhibitors are incubated with PI3Kα, PI3Kγ, PI3Kδ, PI3Kβ, PI3KC2β, mTOR, DNA-PK, ATM kinase and/or PI4KIIIα bound to known radioligands. Detailed conditions for assaying a compound utilized in this invention as an inhibitor of PI3Kα, PI3Kγ, PI3Kδ, PI3Kβ, PI3KC2β, mTOR, DNA-PK, ATM kinase and/or PI4KIIIα, or a mutant thereof, are set forth in the Examples below.

Without wishing to be bound by any particular theory, it is believed that a provided compound comprising a warhead moiety is more effective at inhibiting a PI3 kinase, or a mutant thereof, as compared to a corresponding compound wherein the R¹ moiety of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e is instead a non-warhead group or is completely absent (i.e., is hydrogen). For example, a compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e can be more effective at inhibition of PI3 kinase, or a mutant thereof (for example, Glu542, Glu545 and His1047), as compared to a corresponding compound wherein the R¹ moiety of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e is instead a non-warhead moiety or is absent.

A provided compound comprising a warhead moiety, as disclosed above, can be more potent with respect to an IC₅₀ against a PI3 kinase, or a mutant thereof (for example, Glu542, Glu545 and His1047), than a corresponding compound wherein the R¹ moiety of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e is instead a non-warhead moiety or is absent. Such comparative potency of a compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e as compared to a corresponding compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e wherein the R¹ moiety of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e is instead a non-warhead moiety, can be determined by standard time-dependent assay methods, such as those described in detail in the Examples section, infra. In certain embodiments, a compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e is measurably more potent than a corresponding compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e wherein the R¹ moiety of formula I, II, II-a, II-b, II-c, II-d, II-e, II-f, II-g, II-h, III, IV, V-a, V-b, VI-a, VI-b, VII, VIII, IX, X, XI, XII, XII-a, MI-b, XII-c, XII-d, or XII-e is instead a non-warhead moiety or is absent. In some embodiments, a compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e is measurably more potent, wherein such potency is observed after about 1 minute, about 2 minutes, about 5 minutes, about 10 minutes, about 20 minutes, about 30 minutes, about 1 hour, about 2 hours, about 3 hours, about 4 hours, about 8 hours, about 12 hours, about 16 hours, about 24 hours, or about 48 hours, than a corresponding compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e wherein the R¹ moiety of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e is instead a non-warhead moiety or is absent. In some embodiments, a compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e is any of about 1.5 times, about 2 times, about 5 times, about 10 times, about 20 times, about 25 times, about 50 times, about 100 times, or even about 1000 times more potent than a corresponding compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e wherein the R¹ moiety of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e is instead a non-warhead moiety or is absent. For example, it has been found that compound II-a-16 is about 35 times more potent that its reversible counterpart II^{R}-a-16 in a PI3Kα HTRF assay.

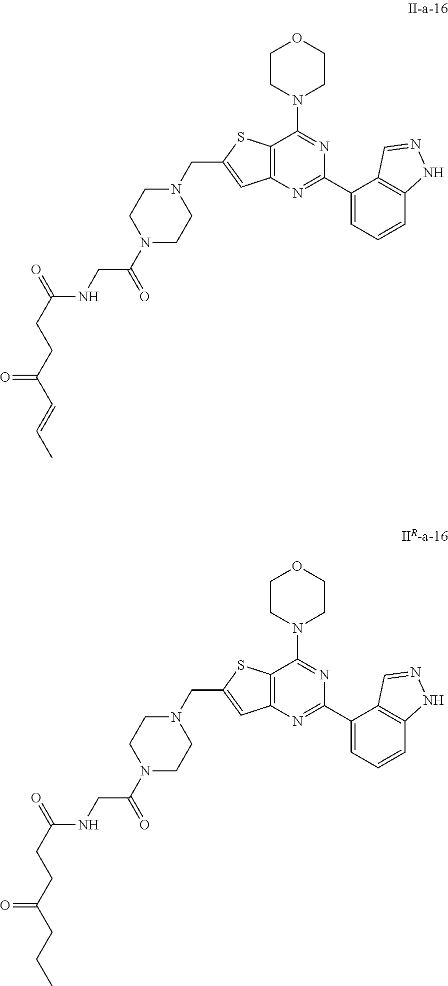

Other examples of the superiority of provided covalent inhibitors over non-covalent inhibitors are shown in Tables 18 and 19 below. “A” designates ≦10 nM; “B” designates 10-100 nM; and “C” designates 100-1000 nM

TABLE 18

| | | EC₅₀ | | |
|---|---|---|---|---|
| Cmpd | Structure | pAkt^{Ser473} | Prolonged PD | Mechanism |
| II-a-148 | 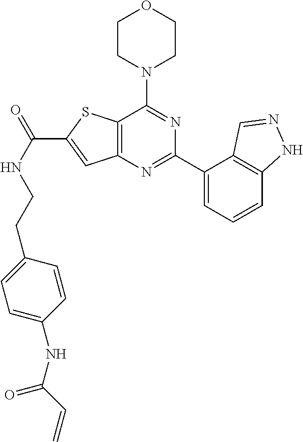 | B | Yes | Irreversible |
| II^{R}-a-148 | 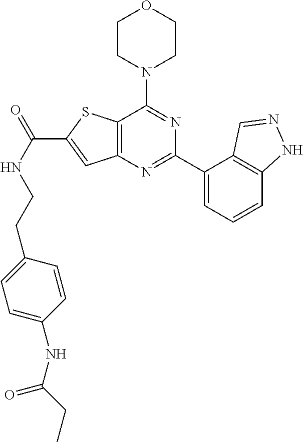 | C | No | Reversible |

TABLE 19

| | | EC₅₀ | | | |
|---|---|---|---|---|---|
| Cmpd | Structure | pAkt^{Ser473} | GI₅₀ | Prolonged PD | Mechanism |
| GDC-941 | 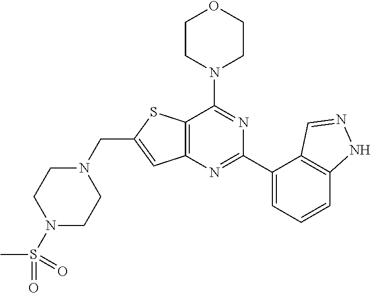 | B | C | No | Reversible |
| II-a-148 | 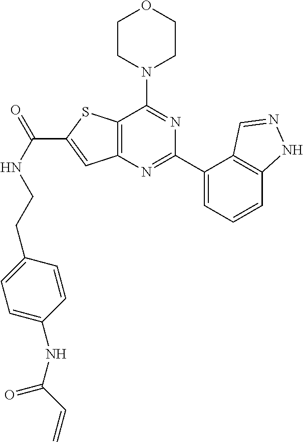 | B | B | Yes | Irreversible |
| II-a-3 | 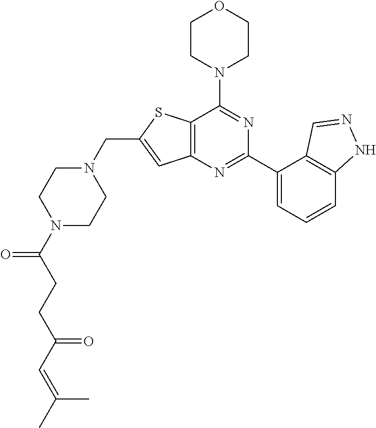 | A | B | Yes | Irreversible |

### PI3K Pathway

The phosphatidylinositol 3-kinase pathway is a central signaling pathway that exerts its effect on numerous cellular functions including cell cycle progression, proliferation, motility, metabolism and survival (Marone, et al. Biochim. Biophys. Acta (2008) 1784: 159-185). Activation of receptor tyrosine kinases in the case of Class IA PI3Ks, or G-proteins in the case of Class IB PI3Kγ, causes phosphorylation of phosphatidylinositol-(4,5)-diphosphate, resulting in membrane-bound phosphatidylinositol-(3,4,5)-triphosphate. The latter promotes the transfer of a variety of protein kinases from the cytoplasm to the plasma membrane by binding of phosphatidylinositol-(3,4,5)-triphosphate to the pleckstrin-homology (PH) domain of the kinase.

Kinases that are downstream targets of PI3K include phosphotidylinositide-dependent kinase 1 (PDK1) and Akt (also known as Protein Kinase B or PKB). Phosphorylation of such kinases then allows for the activation or deactivation of numerous other pathways, involving mediators such as GSK3, mTOR, PRAS40, FKHD, NF-κB, BAD, Caspase-9, and others. These pathways are involved in many cellular processes, such as cell cycle progression, cell survival and apoptosis, cell growth, transcription, translation, metabolism, degranulation, and cell motility.

An important negative feedback mechanism for the PI3K pathway is PTEN, a phosphatase that catalyzes the dephosphorylation of phosphatidylinositol-(3,4,5)-triphosphate to phosphatidylinositol-(4,5)-diphosphate. In more than 60% of all solid tumors, PTEN is mutated into an inactive form, permitting a constitutive activation of the PI3K pathway. As many cancers are solid tumors, such an observation provides evidence that a targeting of PI3K itself or individual downstream kinases in the PI3K pathway provide a promising approach to mitigate or even abolish the disregulation in many cancers and thus restore normal cell function and behavior.

### Class I PI3 Kinases

Because PI3 Kinases (“PI3Ks”) are implicated in cell growth, proliferation, and cell survival, they have been long investigated for their role in the pathogenesis of cancer. The aberrations in PI3K signaling most frequently observed in malignancy are loss or attenuation of PTEN function and mutations in PI3Kα. PTEN dephosphorylates phosphatidylinositol-(3,4,5)-triphosphate and is therefore a negative regulator of the PI3Ks. Loss of PTEN function results in constitutive activity of PI3K and has been implicated in glioma, melanoma, prostate, endometrial, ovarian, breast, and colorectal cancers, as well as leukemia.

Mutations of the PIK3CA gene that codes for PI3Kα are observed in over 30% of solid tumors. The PIK3CA is also amplified in many cancers. Expression of a constitutively active PI3Kα form allows cell survival and migration under suboptimal conditions, leading to tumor formation and metastasis. The overexpression of PI3Kα and/or mutations in PI3Kα have been implicated in a whole host of cancers including, but not limited to, ovarian, cervical, lung, colorectal, gastric, brain, breast and hepatocellular carcinomas.

PI3Kβ has also been implicated in carcinogenesis. The loss of PI3Kβ impedes cell growth of mouse embryonic fibroblasts (Jia, et al., Nature (2008) 454: 776-779). The role of PI3Kβ in tumorigenesis caused by PTEN loss was investigated in prostatic epithelium. Ablation of PI3Kβ in the prostate blocked the tumorigenesis driven by PTEN loss in the anterior prostate. PI3Kβ is an important target for treating solid tumors.

In addition to direct effects, it is believed that activation of Class IA PI3Ks, such as PI3Kα and PI3Kβ, contributes to tumorigenic events that occur upstream in signalling pathways, for example by way of ligand-dependent or ligand-independent activation of receptor tyrosine kinases, GPCR systems or integrins (Vara, et al., Cancer Treatment Reviews (2004) 30: 193-204). Examples of such upstream signalling pathways include over-expression of the receptor tyrosine kinase Erb2 in a variety of tumors leading to activation of PI3K-mediated pathways (Harari, et al., Oncogene (2000) 19: 6102-6114) and over-expression of the oncogene Ras (Kauffmann-Zeh, et al., Nature (1997) 385: 544-548). In addition, Class IA PI3Ks may contribute indirectly to tumorigenesis caused by various downstream signaling events. For example, loss of the effect of the PTEN tumor-suppressor phosphatase that catalyzes conversion of phosphatidylinositide-(3,4,5)-triphosphate back to phosphatidylinositide-(4,5)-diphosphate is associated with a very broad range of tumors via deregulation of PI3K-mediated production of phosphatidylinositide-(3,4,5)-triphosphate (Simpson and Parsons, Exp. Cell Res. (2001) 264: 29-41). Furthermore, augmentation of the effects of other PI3K-mediated signaling events is believed to contribute to a variety of cancers, for example by activation of Akt (Nicholson and Anderson, Cellular Signalling (2002) 381-395).

In addition to a role in mediating proliferative and survival signaling in tumor cells, there is also good evidence that Class IA PI3K enzymes will also contribute to tumorigenesis via its function in tumor-associated stromal cells. For example, PI3K signaling is known to play an important role in mediating angiogenic events in endothelial cells in response to pro-angiogenic factors such as VEGF (Abid, et al., Arterioscler. Thromb. Vasc. Biol. (2004) 24: 294-300). As Class I PI3K enzymes are also involved in motility and migration (Sawyer, Expert Opinion Investig. Drugs (2004) 1-19), PI3K inhibitors should provide therapeutic benefit via inhibition of tumor cell invasion and metastasis.

In addition, Class I PI3K enzymes play an important role in the regulation of immune cells with PI3K activity contributing to pro-tumorigenic effects of inflammatory cells (Coussens and Werb, Nature (2002) 420: 860-867). These findings suggest that pharmacological inhibitors of Class I PI3K enzymes should be of therapeutic value for treatment of the various forms of the disease of cancer comprising solid tumors such as carcinomas and sarcomas and the leukemias and lymphoid malignancies. In particular, inhibitors of Class I PI3K enzymes should be of therapeutic value for treatment of, for example, cancer of the breast, colorectum, lung (including small cell lung cancer, non-small cell lung cancer and bronchioalveolar cancer) and prostate, and of cancer of the bile duct, bone, bladder, head and neck, kidney, liver, gastrointestinal tissue, esophagus, ovary, pancreas, skin, testes, thyroid, uterus, cervix and vulva, and of leukemias (including ALL and CML), multiple myeloma and lymphomas.

PI3K has been linked to the control of cell and organ size. Overexpression of PI3Kα leads to an enlarged heart in the mouse (Shioi et al., EMBO J. (2000) 19: 2537-2548). An even bigger increase in heart size is seen when Akt/PKB, which is downstream of PI3K, is overexpressed. This phenomenon can be reversed by treatment with rapamycin, an inhibitor of mTOR, signifying that Akt/PKB signaling is effected via mTOR to control heart size.

While Class IA PI3Ks, such as PI3Kα, control heart size, mice deficient in PI3Kγ show no effect on heart size. However, PI3Kγ has been shown to influence contractility of the heart. In a transverse aortic constriction (TAC) model, mice deficient in PI3Kγ displayed fibrosis and chamber dilation leading to acute heart failure. PI3Kγ and PI3Kδ have also been shown to regulate infarct size after ischemia/reperfusion injury (Doukas et al., Proc. Natl. Acad. Sci. USA (2006) 103: 19866-19871). For example, treatment of animals with TG100-115, a PI3Kγ/δ dual inhibitor, has been shown to decrease inflammatory responses and edema formation, and is currently being investigated in clinical trials for acute myocardial infarction.

PI3Kγ and PI3Kδ are primarily expressed in leukocytes. Although PI3Kγ and PI3Kδ have been implicated in chronic inflammation and allergy through knockout studies, PI3Kα and PI3Kβ cannot be studied in knockout mice, because mice lacking PI3Kα and PI3Kβ die during embryonic development. PI3Kγ knockout mice display impaired migration of cells important for the inflammatory response, such as neutrophils, macrophages, mast cells, dendritic cells and granulocytes. Mast cells are primary effectors in allergic responses, asthma and atopic dermatitis due to the expression of the high affinity receptor for IgE on their surface. In addition, PI3Kγ knockout mice are protected against systemic anaphylaxis. PI3Kδ inactive mice also display an impaired IgE-mediated inflammatory response, and their mast cells display defective migration.

Inflammatory diseases in which PI3Kγ and PI3Kδ have been implicated include, but are not limited to, rheumatoid arthritis, systemic lupus erythematosus, atherosclerosis, acute pancreatitis, psoriasis, and chronic obstructive pulmonary disease (COPD).

### Class II PI3 Kinases

Class II PI3Ks are characterized by a C-terminal C2 homology domain. Class II comprises three catalytic isoforms: C2α, C2β, and C2γ. C2α and C2β are expressed throughout the body, while C2γ is limited to hepatocytes. No regulatory subunit has been identified for the Class II PI3Ks. Various stimuli have been reported to activate class II PI3Ks, including chemokines (MCP-1), cytokines (leptin and TNFa), LPA, insulin and EGF-, PDGF-, and SCF-receptors. It has been suggested that PI3KC2β may be involved in LPA-induced migration of ovarian and cervical cancer cells (Maffucci, et al., J. Cell. Biol. (2005) 169: 789-799).

### PI4 Kinases

Closely related to the PI3Ks are phophatidylinositol 4-kinases (“PI4Ks”), which phosphorylate the 4′-OH position of phosphatidylinositides. Of the four known PI4K isoforms, PI4KA, also known as PI4KIIIα, is the mostly closely related to PI3Ks. PI4KIIIα is expressed primarily in the nervous system, and is mainly localized to the endoplasmic reticulum, nucleus and plasma membrane. At the plasma membrane, PI4KIIIα associates with ion channels which are involved in cytoskeletal remodeling and membrane blebbing (Kim, et al., EMBO J. (2001) 20: 6347-6358).

### Class IV PI3 Kinases

Mammalian target of rapamycin (mTOR) is a serine/threonine protein kinase that is regulated by growth factors and nutrient availability. mTOR is responsible for coordinating protein synthesis, cell growth and proliferation. Much of the knowledge of mTOR signaling is based on studies with its ligand rapamycin. Rapamycin first binds to the 12 kDa immunophilin FK506-binding protein (FKBP 12) and this complex inhibits mTOR signaling (Tee and Blenis, Seminars in Cell and Developmental Biology. 2005, 16, 29-37). mTOR protein consists of a catalytic kinase domain, an FKBP12-Rapamycin binding (FRB) domain, a putative repressor domain near the C-terminus and up to 20 tandemly-repeated HEAT motifs at the N-terminus, as well as FRAP-ATM-TRRAP (FAT) and FAT C-terminus domain (Huang and Houghton, Curr. Opin. in Pharmacology (2003) 3: 371-377). mTOR kinase is a key regulator of cell growth and has been shown to regulate a wide range of cellular functions including translation, transcription, mRNA turnover, protein stability, actin cytoskeleton reorganization and autophagy (Jacinto and Hall, Nat. Rev. Mol. Cell Bio. (2005) 4: 117-126). mTOR kinase integrates signals from growth factors (such as insulin or insulin-like growth factor) and nutrients (such as amino acids and glucose) to regulate cell growth. mTOR kinase is activated by growth factors through the PDK-Akt pathway. The most well characterized function of mTOR kinase in mammalian cells is regulation of translation through two pathways, namely activation of ribosomal S6K1 to enhance translation of mRNAs that bear a 5′-terminal oligopyrimidine tract (TOP) and suppression of 4E-BP1 to allow CAP-dependent mRNA translation.

There is now considerable evidence indicating that the pathways upstream of mTOR are frequently activated in cancer (Vivanco and Sawyers, Nat. Rev. Cancer (2002) 2: 489-501; Bjornsti and Houghton, Nat. Rev. Cancer (2004) 4: 335-348; Inoki, et al., Nature Genetics (2005) 37: 19-24). For example, components of the PI3K pathway that are mutated in different human tumors include activating mutations of growth factor receptors and the amplification and/or overexpression of PI3K and Akt. In addition, there is evidence that endothelial cell proliferation may also be dependent upon mTOR signaling. Endothelial cell proliferation is stimulated by vascular endothelial cell growth factor (VEGF) activation of the PI3K-Akt-mTOR signalling pathway (Dancey, Expert Opinion on Investigational Drugs, 2005, 14, 313-328). Moreover, mTOR kinase signaling is believed to partially control VEGF synthesis through effects on the expression of hypoxia-inducible factor-1α (HIF-1α) (Hudson, et al., Mol. Cell. Biol. (2002) 22: 7004-7014). Therefore, tumor angiogenesis may depend on mTOR kinase signaling in two ways, through hypoxia-induced synthesis of VEGF by tumour and stromal cells, and through VEGF stimulation of endothelial proliferation and survival through PI3K-Akt-mTOR signalling.

These findings suggest that pharmacological inhibitors of mTOR kinase should be of therapeutic value for treatment of the various forms of the disease of cancer comprising solid tumours such as carcinomas and sarcomas and the leukemias and lymphoid malignancies. In addition to tumorigenesis, there is evidence that mTOR kinase plays a role in an array of hamartoma syndromes. Recent studies have shown that the tumor suppressor proteins such as TSC1, TSC2, PTEN and LKB1 tightly control mTOR kinase signaling. Loss of these tumor suppressor proteins leads to a range of hamartoma conditions as a result of elevated mTOR kinase signaling (Tee and Blenis, Seminars in Cell and Developmental Biology, 2005, 29-37). Syndromes with an established molecular link to dysregulation of mTOR kinase include Peutz-Jeghers syndrome (PJS), Cowden disease, Bannayan-Riley-Ruvalcaba syndrome (BRRS), Proteus syndrome, Lhermitte-Duclos disease and TSC (Inoki, et al., Nature Genetics (2005) 37: 19-24). Patients with these syndromes characteristically develop benign hamartomatous tumors in multiple organs.

Recent studies have revealed a role for mTOR kinase in other diseases (Easton and Houghton, Exp. Opin. Ther. Targets (2004) 8: 551-564). Rapamycin has been demonstrated to be a potent immunosuppressant by inhibiting antigen-induced proliferation of T cells, B cells and antibody production and thus mTOR kinase inhibitors may also be useful immunosuppressives. Inhibition of the kinase activity of mTOR may also be useful in the prevention of restenosis, which is the control of undesired proliferation of normal cells in the vasculature in response to the introduction of stents in the treatment of vasculature disease (Morice, et al., New Engl. J. Med. (2002) 346: 1773-1780). Furthermore, the rapamycin analog, everolimus, can reduce the severity and incidence of cardiac allograft vasculopathy (Eisen, et al., New Engl. J. Med. (2003) 349: 847-858). Elevated mTOR kinase activity has been associated with cardiac hypertrophy, which is of clinical importance as a major risk factor for heart failure and is a consequence of increased cellular size of cardiomyocytes (Tee and Blenis, Seminars in Cell and Developmental Biology, 2005, 29-37). Thus mTOR kinase inhibitors are expected to be of value in the prevention and treatment of a wide variety of diseases in addition to cancer.

Dual inhibition of mTOR and PI3K has been shown to be particularly effective in shutting down cell proliferation that could be responsible in various cancers. A dual inhibitor of mTOR and PI3Kα known as PI-103 was shown to be more effective in blocking proliferation in glioma cells (Fan, et al., Cell Cycle (2006) 5: 2301-2305). A similar effect was seen when a combination therapy of rapamycin, which is an mTOR inhibitor, and PIK90, a pure PI3Ka inhibitor, were used. These results suggest a rationale for combining inhibitors of mTOR and PI3Kα for glioblastoma, and also for the use of dual inhibitors of PI3Kα and mTOR.

Another dual mTOR-PI3K inhibitor is an imidazo[4,5-c]quinoline known as NVP-BEZ235 (Maira, et al., Mol. Cancer Ther. (2008) 7: 1851-1863). NVP-BEZ235 showed efficacy in reduced tumor size in PC3M-tumor bearing mice and achieved tumor stasis in a glioblastoma model. In addition, NVP-BEZ235 given in combination with the standard of care temozolomide caused tumor regression in a glioblastoma model without a significant effect on body weight gain, showing that a dual mTOR-PI3Kα inhibitor can enhance efficacy of other anticancer agents when given in combination. NVP-BEZ235 is currently in clinical trials for cancer treatment.

The DNA-dependent protein kinase (DNA-PK) is a nuclear serine/threonine protein kinase that is activated upon association with DNA. Biochemical and genetic data have revealed this kinase to be composed of a large catalytic subunit, termed DNA-PKcs, and a regulatory component termed Ku. DNA-PK has been shown to be a crucial component of both the DNA double-strand break (DSB) repair machinery and the V(D)J recombination apparatus. In addition, recent work has implicated DNA-PK components in a variety of other processes, including the modulation of chromatin structure and telomere maintenance (Smith and Jackson, Genes and Dev. (1999) 13: 916-934).

DNA DSBs are regarded as the most lethal lesion a cell can encounter. To combat the serious threats posed by DNA DSBs, eukaryotic cells have evolved several mechanisms to mediate their repair. In higher eukaryotes, the predominant of these mechanisms is DNA non-homologous end joining (NHEJ), also known as illegitimate recombination. DNA-PK plays a key role in this pathway. Increased DNA-PK activity has been demonstrated both in vitro and in vivo and correlates with the resistance of tumour cells to IR and bifunctional alkylating agents (Muller, et al., Blood (1998) 92: 2213-2219; Sirzen, et al., Eur. J. Cancer (1999) 35: 111-116). Therefore, increased DNA-PK activity has been proposed as a cellular and tumor resistance mechanism. Hence, inhibition of DNA-PK with a small molecule inhibitor may prove efficacious in tumors where over-expression is regarded as a resistance mechanism.

Given the involvement of DNA-PK in DNA repair processes, and that small molecule inhibitors of DNA-PK have been shown to radio- and chemo-sensitize mammalian cells in culture, an application of specific DNA-PK inhibitory drugs would be to act as agents that will enhance the efficacy of both cancer chemotherapy and radiotherapy. DNA-PK inhibitors may also prove useful in the treatment of retroviral mediated diseases. For example it has been demonstrated that loss of DNA-PK activity severely represses the process of retroviral integration (Daniel, et al., Science (1999) 284: 644-7).

The ATM gene encodes a 370-kDa protein that belongs to the PI3K superfamily which phosphorylates proteins rather than lipids. The 350 amino acid kinase domain at the C-terminus of this protein is the only segment of ATM with an assigned function. Exposure of cells to ionizing radiation (IR) triggers ATM kinase activity and this function is required for arrests in G1, S, and G2 phases of the cell cycle (Shiloh and Kastan, Adv. Cancer Res. (2001) 83: 209-254). The mechanisms by which eukaryotic cells sense DNA strand breaks is unknown, but the rapid induction of ATM kinase activity following IR indicates that it acts at an early stage of signal transduction in mammalian cells (Banin, et al. Science (1998) 281: 1674-1677; Canman, et al. Science (1998) 281: 1677-1679). Transfected ATM is a phosphoprotein that incorporates more phosphate after IR treatment of cells (Lim, et al. Nature (2000) 404: 613-617), suggesting that ATM kinase is itself activated by post-translational modification. Inhibiting ATM for the treatment of neoplasms, particularly cancers associated with decreased p53 function, has been suggested (Morgan, et al. Mol. Cell Biol. (1997) 17: 2020-2029; Hartwell and Kastan, Science (1994) 266: 1821-1828; Kastan, New Engl. J. Med. (1995) 333: 662-663; WO 98/56391).

Agents that target two or more PI3Ks are called pan-PI3K inhibitors. In certain embodiments, provided compounds inhibit one or more of PI3Kα, PI3Kγ, PI3Kδ, PI3Kβ, PI3KC2β, mTOR, DNA-PK, ATM kinase, PI4KIIIα and/or another member of the PI3K superfamily. In some embodiments, provided compounds inhibit two or more of PI3Kα, PI3Kγ, PI3Kδ, PI3Kβ, PI3KC2β, mTOR, DNA-PK, ATM kinase, PI4KIIIα and/or another member of the PI3K superfamily, or a mutant thereof (for example, Glu542, Glu545 and His1047), and are therefore pan-PI3K inhibitors. In certain embodiments, a pan-PI3K inhibitor inhibits two or more of PI3Kα, PI3Kγ, PI3Kδ, and PI3Kβ. In certain embodiments, a pan-PI3K inhibitor inhibits three or more of PI3Kα, PI3Kγ, PI3Kδ, and PI3Kβ. In certain embodiments, a pan-PI3K inhibitor inhibits PI3Kα, PI3Kγ, PI3Kδ, and PI3Kβ.

Wortmannin is a natural product that is a pan-PI3K inhibitor. In addition to the classical PI3Ks, wortmannin also inhibits DNA-PK, mTOR, ATR, ATM, PI4K and polo-like kinase (PLK). While wortmannin itself is too toxic to use therapeutically, modified versions of wortmannin have been discovered that show decreased toxicity as compared to wortmannin. One such compound is PX-866, which attenuated growth of a tumor xenograft in mice at around 10 mg/kg (Ihle, et al., Mol. Cancer Ther. (2004) 3: 763-772).

IC87114, a selective inhibitor of PI3Kγ, has shown effects on neutrophil migration (Sadhu, et al., J. Immunol. (2003) 170: 2647-2654) and TNF1α-stimulated elastase exocytosis from neutrophils in an inflammation model (Sadhu, et al., Biochem. Biophys. Res. Commun. (2003) 308: 764-769). IC87114 has also been shown to inhibit acute myeloid leukemia cell proliferation and survival (Billottet, et al., Oncogene (2006) 25: 6648-6659).

TGX-221 is a selective inhibitor of PI3Kβ, and is an analog of the pan-PI3K inhibitor LY294002 (Jackson, et al., Nat. Med. (2005) 11: 507-514). TGX-221 has been shown to interfere with stress-induced phosphatidylinositol-3,4-diphosphate production and integrin α_{IIb}β₃-mediated adhesion in platelets. These results suggest that TGX-221 or other inhibitors of PI3Kβ could have an anti-thrombotic effect in vivo.

PI-103 is a pan-PI3K inhibitor and displays dual inhibition PI3K/mTOR. PI-103 has been shown to attenuate proliferation of glioma, breast, ovarian and cervical tumor cells in mouse xenograft models (Raynaud, et al., Cancer Res. (2007) 67: 5840-5850).

AS-252424, AS-604850 and AS-605240 are selective PI3Kγ inhibitors that have been used to block neutrophil chemotaxis. These compounds have been shown to minimize progression of joint destruction in a rheumatoid arthritis model (Camps, et al., Nat. Med. (2005) 11: 936-943).

ZSTK474 is a PI3K inhibitor that was selected for its ability to block tumor growth. ZSTK474 displayed a strong anti-tumoral activity in a mouse xenograft model (Yaguchi, et al., J. Natl. Cancer Inst. (2006) 98: 545-556).

XL765 and XL147, quinoxaline compounds that are dual PI3K/mTOR inhibitors, have shown efficacy in xenograft models both as single agents as well as in combination with standard chemotherapy. Both compounds are currently in clinical trials for treatment of solid tumors.

SF1126 is a pan-PI3K inhibitor which has entered clinical trials to target cell growth, proliferation and angiogenesis. SF1126 has demonstrated promising in vivo activity in a variety of mouse cancer models, including prostate, breast, ovarian, lung, multiple myeloma, brain and other cancers.

Neurofibromatosis type I (NF1) is a dominantly inherited human disease affecting one in 2500-3500 individuals. Several organ systems are affected, including bones, skin, iris, and the central nervous system, as manifested in learning disabilities and gliomas. A hallmark of NF1 is the development of benign tumors of the peripheral nervous system (neurofibromas), which vary greatly in both number and size among patients. Neurofibromas are heterogeneous tumors composed of Schwann cells, neurons, fibroblasts and other cells, with Schwann cells being the major (60-80%) cell type. PI3K has been implicated in NF1 (Yang, et al. J. Clin. Invest. 116: 2880 (2006).

Schwannomas are peripheral nerve tumors comprised almost entirely of Schwann-like cells, and typically have mutations in the neurofibromatosis type II (NF2) tumor suppressor gene. Ninety percent of NF2 patients develop bilateral vestibular schwannomas and/or spinal schwannomas. Enlarging schwannomas can compress adjacent structures, resulting in deafness and other neurologic problems. Surgical removal of these tumors is difficult, often resulting in increased patient morbidity. PI3K has also been implicated in NF2, suggesting that PI3K inhibitors could be used to treat NF2-related disorders. See Evans, et al., Clin. Cancer Res. 15: 5032 (2009); James, et al. Mol. Cell. Biol. 29: 4250 (2009); Lee et al. Eur. J. Cancer 45: 1709.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are inhibitors of one of more of PI3Kα, PI3Kγ, PI3Kδ, PI3Kβ, PI3KC2β, mTOR, DNA-PK, ATM kinase and/or PI4KIIIα and are therefore useful for treating one or more disorders associated with activity of one or more of PI3Kα, PI3Kγ, PI3Kδ, PI3Kβ, PI3KC2β, mTOR, DNA-PK, ATM kinase and/or PI4KIIIα. Thus, in certain embodiments, the present invention provides a method for treating a PI3Kα-mediated, a PI3Kγ-mediated, a PI3Kδ-mediated, a PI3Kβ-mediated, a PI3KC2β-mediated, an mTOR-mediated, a DNA-PK-mediated, an ATM-mediated and/or a PI4KIIIα-mediated disorder comprising the step of administering to a patient in need thereof a compound of the present invention, or pharmaceutically acceptable composition thereof.

As used herein, the terms “PI3Kα-mediated”, “PI3Kγ-mediated”, “PI3Kδ-mediated”, “PI3Kβ-mediated”, “PI3KC2β-mediated”, “mTOR-mediated”, “DNA-PK-mediated”, “ATM-mediated” and/or “PI4KIIIα-mediated” disorders, diseases, and/or conditions as used herein means any disease or other deleterious condition in which one or more of PI3Kα, PI3Kγ, PI3Kδ, PI3Kβ, PI3KC2β, mTOR, DNA-PK, ATM kinase and/or PI4KIIIα, or a mutant thereof, are known to play a role. Accordingly, another embodiment of the present invention relates to treating or lessening the severity of one or more diseases in which one or more of PI3Kα, PI3Kγ, PI3Kδ, PI3Kβ, PI3KC2β, mTOR, DNA-PK, ATM kinase and/or PI4KIIIα, or a mutant thereof, are known to play a role.

In some embodiments, the present invention provides a method for treating one or more disorders, diseases, and/or conditions wherein the disorder, disease, or condition is a cancer, a neurodegenative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, or a CNS disorder.

Diseases and conditions treatable according to the methods of this invention include, but are not limited to, cancer, neurofibromatosis, ocular angiogenesis, stroke, diabetes, hepatomegaly, cardiovascular disease, Alzheimer's disease, cystic fibrosis, viral disease, autoimmune diseases, atherosclerosis, restenosis, psoriasis, allergic disorders, inflammation, neurological disorders, angiogenic disorders, a hormone-related disease, conditions associated with organ transplantation, immunodeficiency disorders, destructive bone disorders, proliferative disorders, infectious diseases, conditions associated with cell death, thrombin-induced platelet aggregation, chronic myelogenous leukemia (CML), chronic lymphocytic leukemia (CLL), liver disease, pathologic immune conditions involving T cell activation, and CNS disorders in a patient. In one embodiment, a human patient is treated with a compound of the current invention and a pharmaceutically acceptable carrier, adjuvant, or vehicle, wherein said compound of is present in an amount to measurably inhibit PI3 kinase activity.

Compounds of the current invention are useful in the treatment of a proliferative disease selected from a benign or malignant tumor, carcinoma of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia. Other diseases include Cowden syndrome, Lhermitte-Dudos disease and Bannayan-Zonana syndrome, or diseases in which the PI3K/PKB pathway is aberrantly activated.

In certain embodiments, the present invention provides a method for treating or lessening the severity of neurofibromatosis type I (NF1), neurofibromatosis type II (NF2), Schwann cell neoplasms (e.g. malignant peripheral nerve sheath tumors (MPNST's)), or Schwannomas.

Compounds according to the invention are useful in the treatment of inflammatory or obstructive airways diseases, resulting, for example, in reduction of tissue damage, airways inflammation, bronchial hyperreactivity, remodeling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics.

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, such as therapy for or intended to restrict or abort symptomatic attack when it occurs, for example antiinflammatory or bronchodilatory. Prophylactic benefit in asthma may in particular be apparent in subjects prone to “morning dipping”. “Morning dipping” is a recognized asthmatic syndrome, common to a substantial percentage of asthmatics and characterised by asthma attack, e.g. between the hours of about 4 to 6 am, i.e. at a time normally substantially distant form any previously administered symptomatic asthma therapy.

Compounds of the current invention can be used for other inflammatory or obstructive airways diseases and conditions to which the present invention is applicable and include acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy. The invention is also applicable to the treatment of bronchitis of whatever type or genesis including, but not limited to, acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis.

With regard to their anti-inflammatory activity, in particular in relation to inhibition of eosinophil activation, compounds of the invention are also useful in the treatment of eosinophil related disorders, e.g. eosinophilia, in particular eosinophil related disorders of the airways (e.g. involving morbid eosinophilic infiltration of pulmonary tissues) including hypereosinophilia as it effects the airways and/or lungs as well as, for example, eosinophil-related disorders of the airways consequential or concomitant to Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction.

Compounds of the invention are also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, and other inflammatory or allergic conditions of the skin.

Compounds of the invention may also be used for the treatment of other diseases or conditions, such as diseases or conditions having an inflammatory component, for example, treatment of diseases and conditions of the eye such as conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy).

Cardiovascular diseases which can be treated according to the methods of this invention include, but are not limited to, restenosis, cardiomegaly, atherosclerosis, myocardial infarction, ischemic stroke and congestive heart failure.

Neurodegenerative disease which can be treated according to the methods of this invention include, but are not limited to, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

Compounds according to the invention are useful for inhibiting angiogenesis. Angiogenesis refers to the growth of new blood vessels, and is an important contributor to a number of pathological conditions. For example, the role of angiogenesis in promoting and supporting the growth and viability of solid tumors is well documented. Angiogenesis also contributes to other pathological conditions, such as psoriasis and asthma, and pathological conditions of the eye, such as the wet form of age-related macular degeneration (AMD), diabetic retinopathy, diabetic macular edema, and retinopathy of prematurity. PI3K proteins are pro-angiogenic (Graupera et al. Nature (2008) 453(7195):662-6) and thus the subject compounds provide advantages for inhibiting angiogenesis, for example, to treat eye disease associated with ocular angiogenesis, such as by topical administration of the subject compounds. Compounds according to the invention can be formulated for topical administration. For example, the irreversible inhibitor can be formulated for topical delivery to the lung (e.g., as an aerosol, such as a dry powder or liquid formulation) to treat asthma, as a cream, ointment, lotion or the like for topical application to the skin to treat psoriasis, or as an ocular formulation for topical application to the eye to treat an ocular disease. Such a formulation will contain a subject inhibitor and a pharmaceutically acceptable carrier. Additional components, such as preservatives, and agents to increase viscosity of the formulation such as natural or synthetic polymers may also be present. The ocular formulation can be in any suitable form, such as a liquid, an ointment, a hydrogel or a powder. Compounds of the current invention can be administered together with another therapeutic agent, such as an anti-VEGF agent, for example ranibizumab a Fab fragment of an antibody that binds VEGFA, or another anti-angiogenic compound as described further below.

Furthermore, the invention provides the use of a compound according to the definitions herein, or a pharmaceutically acceptable salt, or a hydrate or solvate thereof for the preparation of a medicament for the treatment of a proliferative disease, an inflammatory disease or an obstructive respiratory disease, a cardiovascular disease, a neurological disease, an angiogenic disorder, or a disorder commonly occurring in connection with transplantation.

The compounds and compositions, according to the method of the present invention, may be administered using any amount and any route of administration effective for treating or lessening the severity of cancer, an autoimmune disorder, a proliferative disorder, an inflammatory disorder, a neurodegenerative or neurological disorder, an angiogenic disorder, schizophrenia, a bone-related disorder, liver disease, or a cardiac disorder. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

In some embodiments, a provided composition is administered to a patient in need thereof once daily. Without wishing to be bound by any particular theory, it is believed that prolonged duration of action of an irreversible inhibitor of one or more PI3 kinases is particularly advantageous for once daily administration to a patient in need thereof for the treatment of a disorder associated with one or more PI3 kinases. In certain embodiments, a provided composition is administered to a patient in need thereof at least once daily. In other embodiments, a provided composition is administered to a patient in need thereof twice daily, three times daily, or four times daily.

In certain embodiments, compounds of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, and XII-e, for example, generally provide prolonged duration of action when administered to a patient as compared to a corresponding compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e wherein the R¹ moiety of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e is instead a non-warhead moiety or is absent. For example, a compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e can provide prolonged duration of action when administered to a patient as compared to a corresponding compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e wherein the R¹ moiety of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e is instead a non-warhead moiety or is absent.

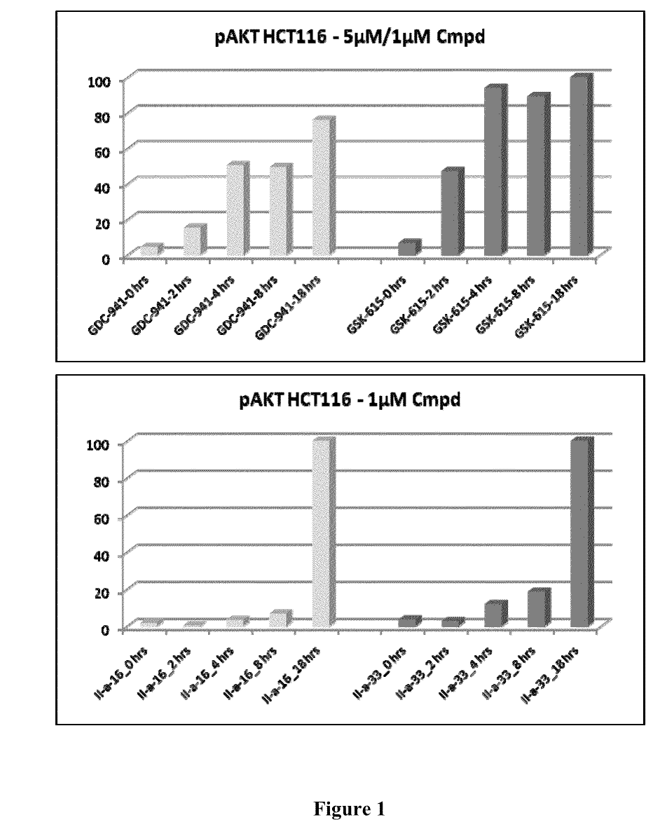

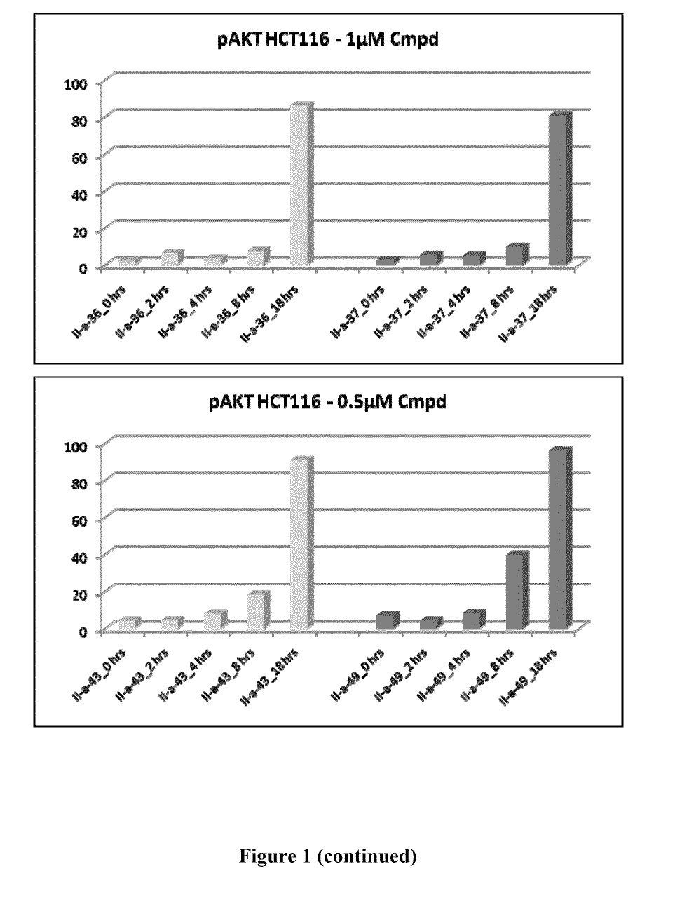

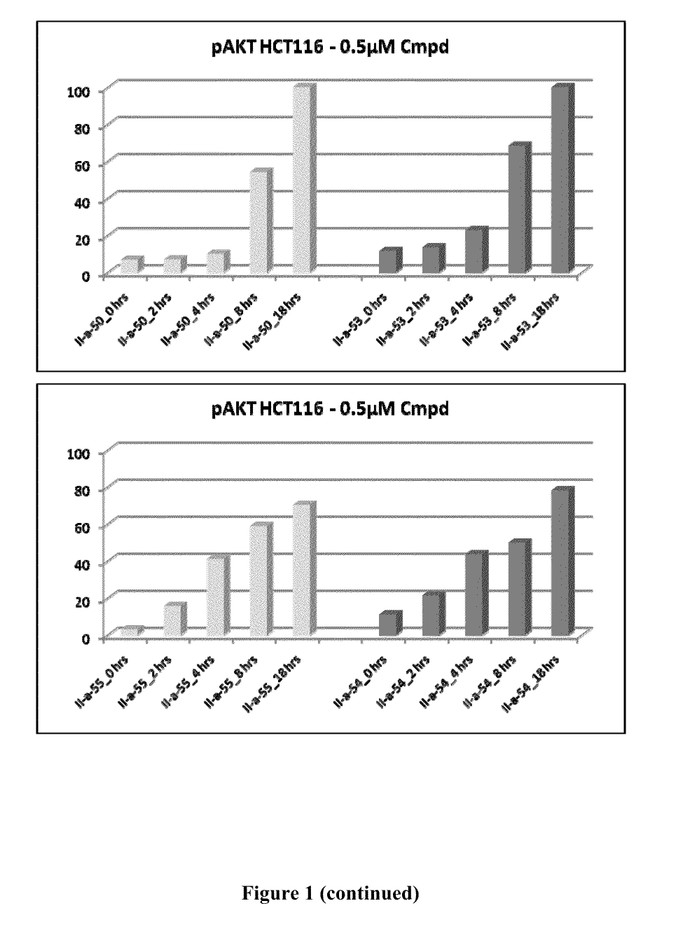

Compounds II-a-16, II-a-33, II-a-36, II-a-27, II-a-43, II-a-49, II-a-50, II-a-53, II-a-54, and II-a-55 were compared with reversible inhibitors GSK-615 and GDC-941 in a HCT116 washout experiment. The results of the study are shown in FIG. 1. Irreversible inhibitors comprising a warhead moiety inhibited PI3Kα for substantially longer periods of time than the reversible inhibitors GSK-615 and GDC-941. In many cases, PI3Kα was inhibited by provided irreversible inhibitors for at least 4 hours. In some cases, PI3Kα was inhibited by provided irreversible inhibitors for at least 8 hours. Without wishing to be bound by any particular theory, it is believed that the prolonged duration of action of provided irreversible inhibitors in vitro in comparison with corresponding reversible inhibitors will translate to a prolonged duration of action in vivo.

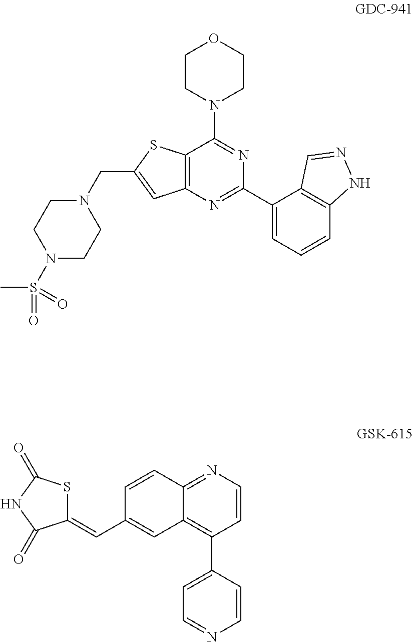

Other reversible inhibitors used as reference compounds in the examples herein include the following:

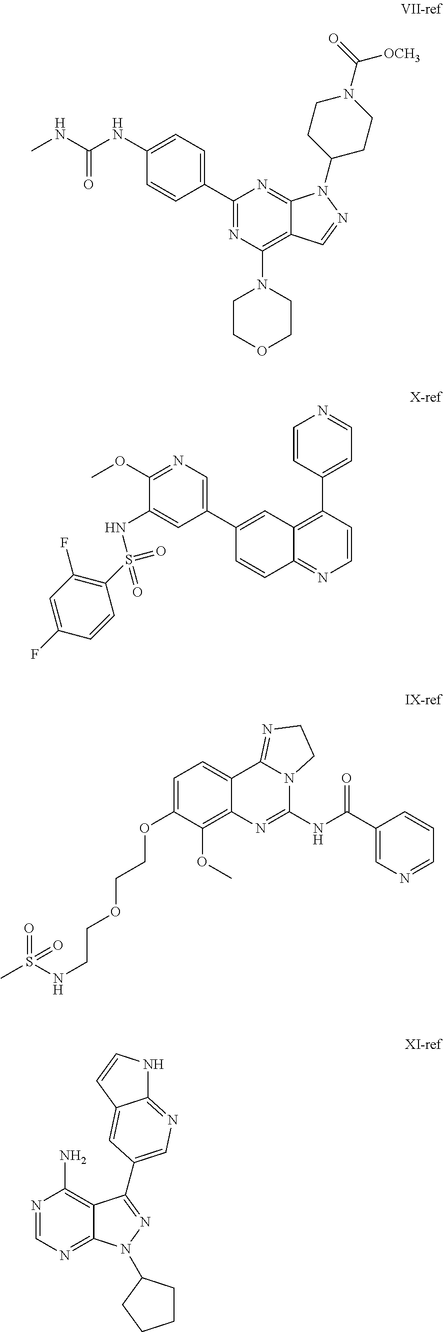

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of inhibiting protein kinase activity in a biological sample comprising the step of contacting said biological sample with a compound of this invention, or a composition comprising said compound.

According to another embodiment, the invention relates to a method of inhibiting PI3Kα, PI3Kγ, PI3Kδ, PI3Kβ, PI3KC2β, mTOR, DNA-PK, ATM kinase and/or PI4KIIIα, or a mutant thereof (for example, Glu542, Glu545 and His1047), activity in a biological sample comprising the step of contacting said biological sample with a compound of this invention, or a composition comprising said compound. In certain embodiments, the invention relates to a method of irreversibly inhibiting PI3Kα, PI3Kγ, PI3Kδ, PI3Kβ, PI3KC2β, mTOR, DNA-PK, ATM kinase and/or PI4KIIIα, or a mutant thereof, activity in a biological sample comprising the step of contacting said biological sample with a compound of this invention, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of protein kinase, or a protein kinase selected from PI3Kα, PI3Kγ, PI3Kδ, PI3Kβ, PI3KC2β, mTOR, DNA-PK, ATM kinase and/or PI4KIIIα, or a mutant thereof, activity in a biological sample is useful for a variety of purposes that are known to one of skill in the art. Examples of such purposes include, but are not limited to, blood transfusion, organ-transplantation, biological specimen storage, and biological assays.

Another embodiment of the present invention relates to a method of inhibiting protein kinase activity in a patient comprising the step of administering to said patient a compound of the present invention, or a composition comprising said compound.

According to another embodiment, the invention relates to a method of inhibiting one or more of PI3Kα, PI3Kγ, PI3Kδ, PI3Kβ, PI3KC2β, mTOR, DNA-PK, ATM kinase and/or PI4KIIIα, or a mutant thereof (for example, Glu542, Glu545 and His1047), activity in a patient comprising the step of administering to said patient a compound of the present invention, or a composition comprising said compound. According to certain embodiments, the invention relates to a method of irreversibly inhibiting one or more of PI3Kα, PI3Kγ, PI3Kδ, PI3Kβ, PI3KC2β, mTOR, DNA-PK, ATM kinase and/or PI4KIIIα, or a mutant thereof (for example, Glu542, Glu545 and His1047), activity in a patient comprising the step of administering to said patient a compound of the present invention, or a composition comprising said compound. In other embodiments, the present invention provides a method for treating a disorder mediated by one or more of PI3Kα, PI3Kγ, PI3Kδ, PI3Kβ, PI3KC2β, mTOR, DNA-PK, ATM kinase and/or PI4KIIIα, or a mutant thereof (for example, Glu542, Glu545 and His1047), in a patient in need thereof, comprising the step of administering to said patient a compound according to the present invention or pharmaceutically acceptable composition thereof. Such disorders are described in detail herein.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

A compound of the current invention may also be used to advantage in combination with other antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin. The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, vinflunine, and vinorelbine; discodermolides; cochicine and epothilones and derivatives thereof. Paclitaxel is marketed under the trade name Taxol™ and Abraxane®. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK) including staurosporine derivatives, such as midostaurin; examples of further compounds include UCN-01, safingol, BAY 43-9006, Bryostatin 1, Perifosine; Ilmofosine; RO 318220 and RO 320432; GO 6976; Isis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives; and m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF.

Further anti-angiogenic compounds include compounds having another mechanism for their activity, e.g. unrelated to protein or lipid kinase inhibition e.g. thalidomide (Thalomid™) and TNP-470.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both, an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-100 mg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### 5. Probe Compounds

In certain aspects, a compound of the present invention may be tethered to a detectable moiety to form a probe compound. In one aspect, a probe compound of the invention comprises an irreversible kinase inhibitor of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e, as described herein, a detectable moiety, and a tethering moiety that attaches the inhibitor to the detectable moiety.

In some embodiments, such probe compounds of the present invention comprise a provided compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e tethered to a detectable moiety, R^{p}, by a bivalent tethering moiety, -T^{p}-. The tethering moiety may be attached to a compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e via any substitutable carbon or nitrogen on the molecule or via R¹. One of ordinary skill in the art will appreciate that when a tethering moiety is attached to R¹, R¹ is a bivalent warhead group denoted as R^{1′}.

In certain embodiments, a provided probe compound is selected from any of formula XIII, XIV, XIV-a, XIV-b, XIV-c, XIV-d, XIV-e, XIV-f, XIV-g, XIV-h, XV, XVI, XVII-a, XVII-b, XVIII-a, XVIII-b, XIX, XX, XXI, XXII, XXIII, XXIV, XXIV-a, XXIV-b, XXIV-c, XXIV-d, and XXIV-e:

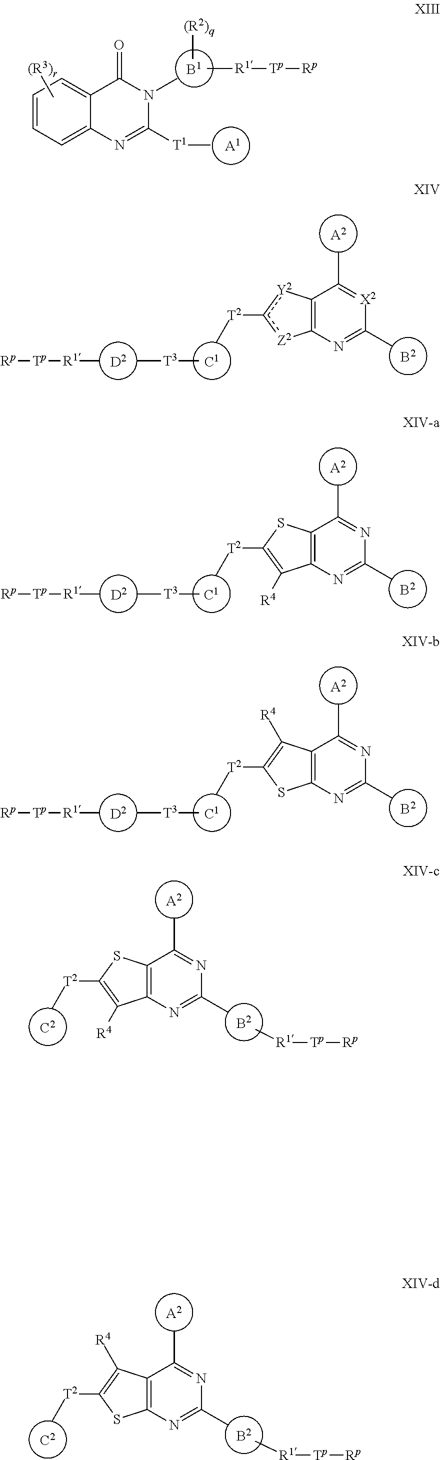

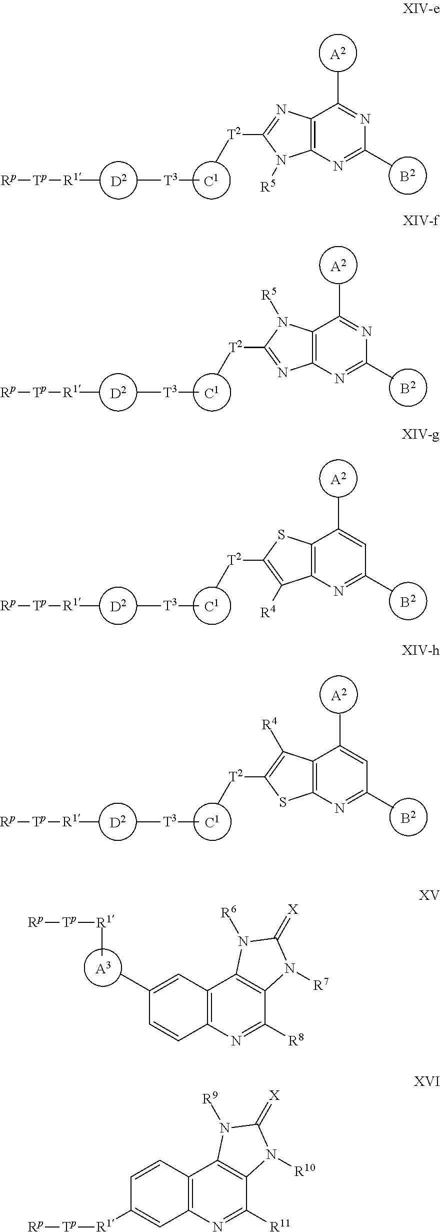

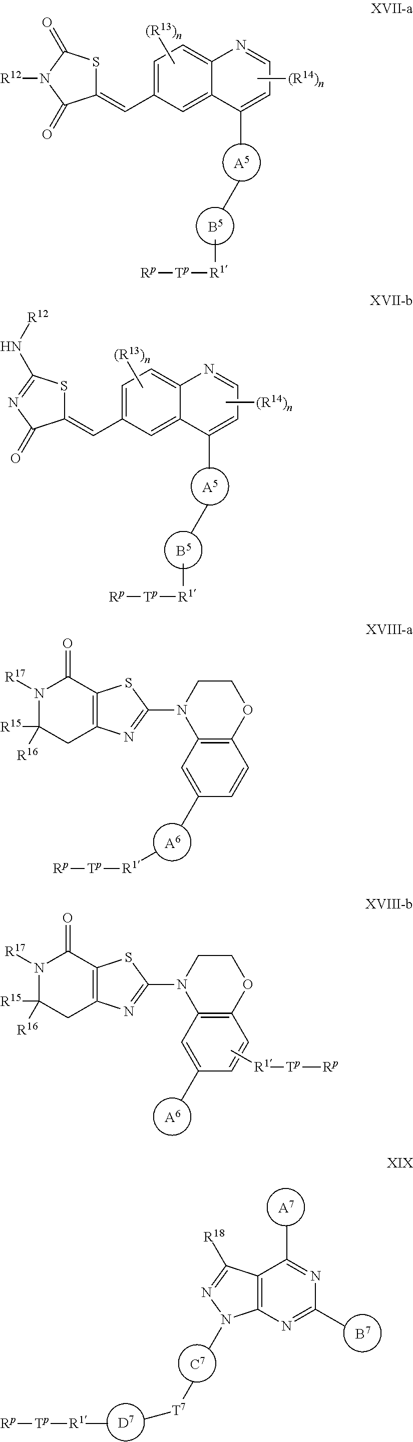

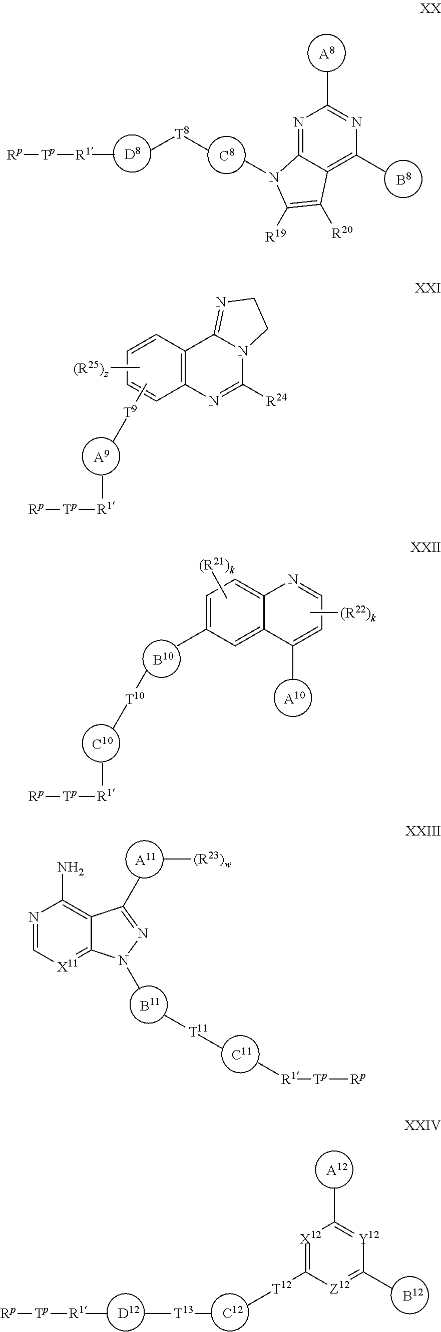

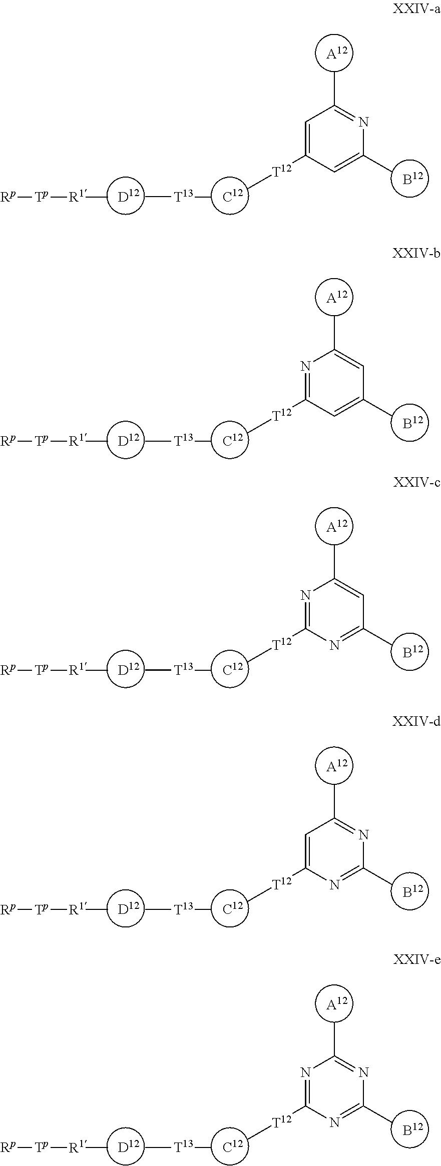

wherein each variable is as defined above with respect to formulae I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, and XII-e, and described in classes and subclasses herein, R^{1′} is a bivalent warhead group, T^{p} is a bivalent tethering moiety; and R^{p} is a detectable moiety.

In some embodiments, R^{p} is a detectable moiety selected from a primary label or a secondary label. In certain embodiments, R^{p} is a detectable moiety selected from a fluorescent label (e.g., a fluorescent dye or a fluorophore), a mass-tag, a chemiluminescent group, a chromophore, an electron dense group, or an energy transfer agent.

As used herein, the term “detectable moiety” is used interchangeably with the term “label” and “reporter” and relates to any moiety capable of being detected, e.g., primary labels and secondary labels. A presence of a detectable moiety can be measured using methods for quantifying (in absolute, approximate or relative terms) the detectable moiety in a system under study. In some embodiments, such methods are well known to one of ordinary skill in the art and include any methods that quantify a reporter moiety (e.g., a label, a dye, a photocrosslinker, a cytotoxic compound, a drug, an affinity label, a photoaffinity label, a reactive compound, an antibody or antibody fragment, a biomaterial, a nanoparticle, a spin label, a fluorophore, a metal-containing moiety, a radioactive moiety, quantum dot(s), a novel functional group, a group that covalently or noncovalently interacts with other molecules, a photocaged moiety, an actinic radiation excitable moiety, a ligand, a photoisomerizable moiety, biotin, a biotin analog (e.g., biotin sulfoxide), a moiety incorporating a heavy atom, a chemically cleavable group, a photocleavable group, a redox-active agent, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, and any combination of the above).

Primary labels, such as radioisotopes (e.g., tritium, ³²P, ³³P, ³⁵S, ¹⁴C, ¹²³I, ¹²⁴I, ¹²⁵I, or ¹³¹I), mass-tags including, but not limited to, stable isotopes (e.g., ¹³C, ²H, ¹⁷O, ¹⁸O, ¹⁵N, ¹⁹F, and ¹²⁷I), positron emitting isotopes (e.g., ¹¹C, ¹⁸F, ¹³N, ¹²⁴I, and ¹⁵O), and fluorescent labels are signal generating reporter groups which can be detected without further modifications. Detectable moities may be analyzed by methods including, but not limited to fluorescence, positron emission tomography, SPECT medical imaging, chemiluminescence, electron-spin resonance, ultraviolet/visible absorbance spectroscopy, mass spectrometry, nuclear magnetic resonance, magnetic resonance, flow cytometry, autoradiography, scintillation counting, phosphoimaging, and electrochemical methods.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 493/503, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X, 5(6)-Carboxyfluorescein, 2,7-Dichlorofluorescein, N,N-Bis(2,4,6-trimethylphenyl)-3,4:9,10-perylenebis(dicarboximide, HPTS, Ethyl Eosin, DY-490XL MegaStokes, DY-485XL MegaStokes, Adirondack Green 520, ATTO 465, ATTO 488, ATTO 495, YOYO-1,5-FAM, BCECF, dichlorofluorescein, rhodamine 110, rhodamine 123, YO-PRO-1, SYTOX Green, Sodium Green, SYBR Green I, Alexa Fluor 500, FITC, Fluo-3, Fluo-4, fluoro-emerald, YoYo-1 ssDNA, YoYo-1 dsDNA, YoYo-1, SYTO RNASelect, Diversa Green-FP, Dragon Green, EvaGreen, Surf Green EX, Spectrum Green, NeuroTrace 500525, NBD-X, MitoTracker Green FM, LysoTracker Green DND-26, CBQCA, PA-GFP (post-activation), WEGFP (post-activation), FLASH-CCXXCC, Azami Green monomeric, Azami Green, green fluorescent protein (GFP), EGFP (Campbell Tsien 2003), EGFP (Patterson 2001), Kaede Green, 7-Benzylamino-4-Nitrobenz-2-Oxa-1,3-Diazole, Bexl, Doxorubicin, Lumio Green, and SuperGlo GFP.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags. Stable isotopes (e.g., ¹³C, ²H, ¹⁷O, ¹⁸O and ¹⁵N) may also be used as mass-tags.

The term “chemiluminescent group,” as used herein, refers to a group which emits light as a result of a chemical reaction without the addition of heat. By way of example, luminol (5-amino-2,3-dihydro-1,4-phthalazinedione) reacts with oxidants like hydrogen peroxide (H₂O₂) in the presence of a base and a metal catalyst to produce an excited state product (3-aminophthalate, 3-APA).

The term “chromophore,” as used herein, refers to a molecule which absorbs light of visible wavelengths, UV wavelengths or IR wavelengths.

The term “dye,” as used herein, refers to a soluble, coloring substance which contains a chromophore.

The term “electron dense group,” as used herein, refers to a group which scatters electrons when irradiated with an electron beam. Such groups include, but are not limited to, ammonium molybdate, bismuth subnitrate, cadmium iodide, carbohydrazide, ferric chloride hexahydrate, hexamethylene tetramine, indium trichloride anhydrous, lanthanum nitrate, lead acetate trihydrate, lead citrate trihydrate, lead nitrate, periodic acid, phosphomolybdic acid, phosphotungstic acid, potassium ferricyanide, potassium ferrocyanide, ruthenium red, silver nitrate, silver proteinate (Ag Assay: 8.0-8.5%) “Strong”, silver tetraphenylporphin (S-TPPS), sodium chloroaurate, sodium tungstate, thallium nitrate, thiosemicarbazide (TSC), uranyl acetate, uranyl nitrate, and vanadyl sulfate.

The term “energy transfer agent,” as used herein, refers to a molecule which either donates or accepts energy from another molecule. By way of example only, fluorescence resonance energy transfer (FRET) is a dipole-dipole coupling process by which the excited-state energy of a fluorescence donor molecule is non-radiatively transferred to an unexcited acceptor molecule which then fluorescently emits the donated energy at a longer wavelength.

The term “moiety incorporating a heavy atom,” as used herein, refers to a group which incorporates an ion of atom which is usually heavier than carbon. In some embodiments, such ions or atoms include, but are not limited to, silicon, tungsten, gold, lead, and uranium.

The term “photoaffinity label,” as used herein, refers to a label with a group, which, upon exposure to light, forms a linkage with a molecule for which the label has an affinity.

The term “photocaged moiety,” as used herein, refers to a group which, upon illumination at certain wavelengths, covalently or non-covalently binds other ions or molecules.

The term “photoisomerizable moiety,” as used herein, refers to a group wherein upon illumination with light changes from one isomeric form to another.

The term “radioactive moiety,” as used herein, refers to a group whose nuclei spontaneously give off nuclear radiation, such as alpha, beta, or gamma particles; wherein, alpha particles are helium nuclei, beta particles are electrons, and gamma particles are high energy photons.

The term “spin label,” as used herein, refers to molecules which contain an atom or a group of atoms exhibiting an unpaired electron spin (i.e. a stable paramagnetic group) that in some embodiments are detected by electron spin resonance spectroscopy and in other embodiments are attached to another molecule. Such spin-label molecules include, but are not limited to, nitryl radicals and nitroxides, and in some embodiments are single spin-labels or double spin-labels.

The term “quantum dots,” as used herein, refers to colloidal semiconductor nanocrystals that in some embodiments are detected in the near-infrared and have extremely high quantum yields (i.e., very bright upon modest illumination).

One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering moiety, such as a bivalent saturated or unsaturated hydrocarbon chain.

In some embodiments, detectable moieties are attached to a provided compound via click chemistry. In some embodiments, such moieties are attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41, 2596-99 and Sun et al., Bioconjugate Chem., 2006, 17, 52-57. In some embodiments, a click ready inhibitor moiety is provided and reacted with a click ready -T^{p}-R^{p} moiety. As used herein, “click ready” refers to a moiety containing an azide or alkyne for use in a click chemistry reaction. In some embodiments, the click ready inhibitor moiety comprises an azide. In certain embodiments, the click ready -T^{p}-R^{p} moiety comprises a strained cyclooctyne for use in a copper-free click chemistry reaction (for example, using methods described in Baskin et al., Proc. Natl. Acad. Sci. USA 2007, 104, 16793-16797).

In certain embodiments, the click ready inhibitor moiety is of one of the following formulae:

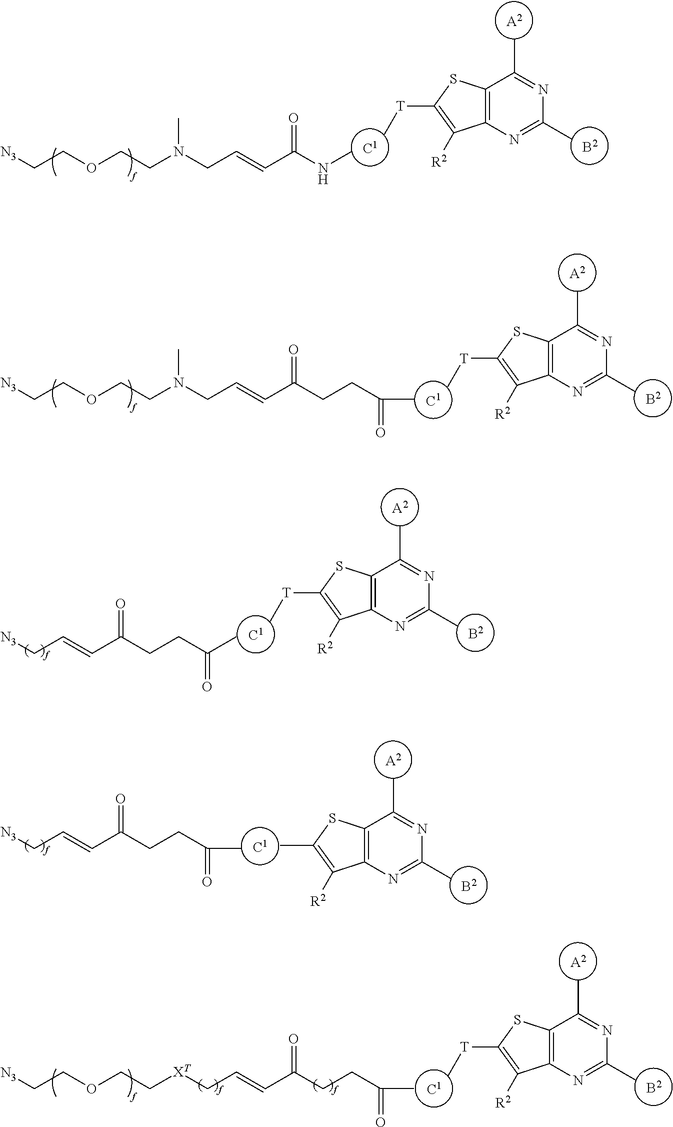

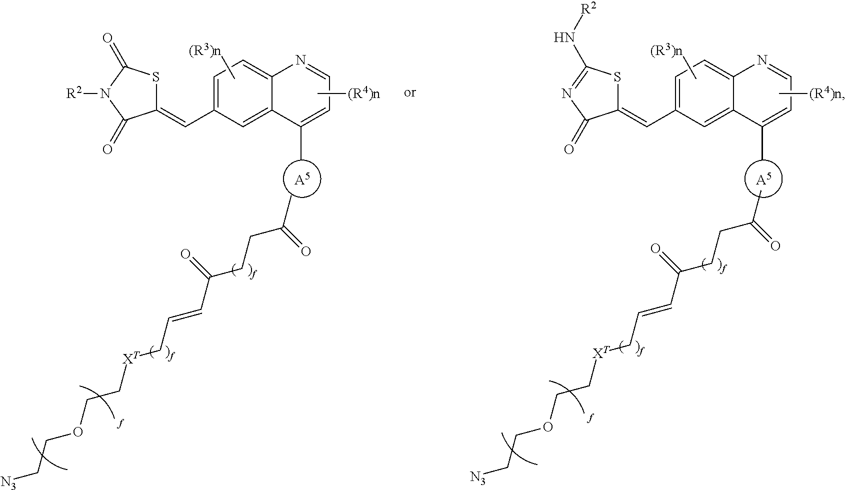

wherein the variables are as defined above with respect to Formulae II-a, V-a, and V-b and described herein, XT is —O—, —NH—, or —NMe-, and each occurrence off is independently 1, 2, or 3.

Exemplary click ready inhibitors include:

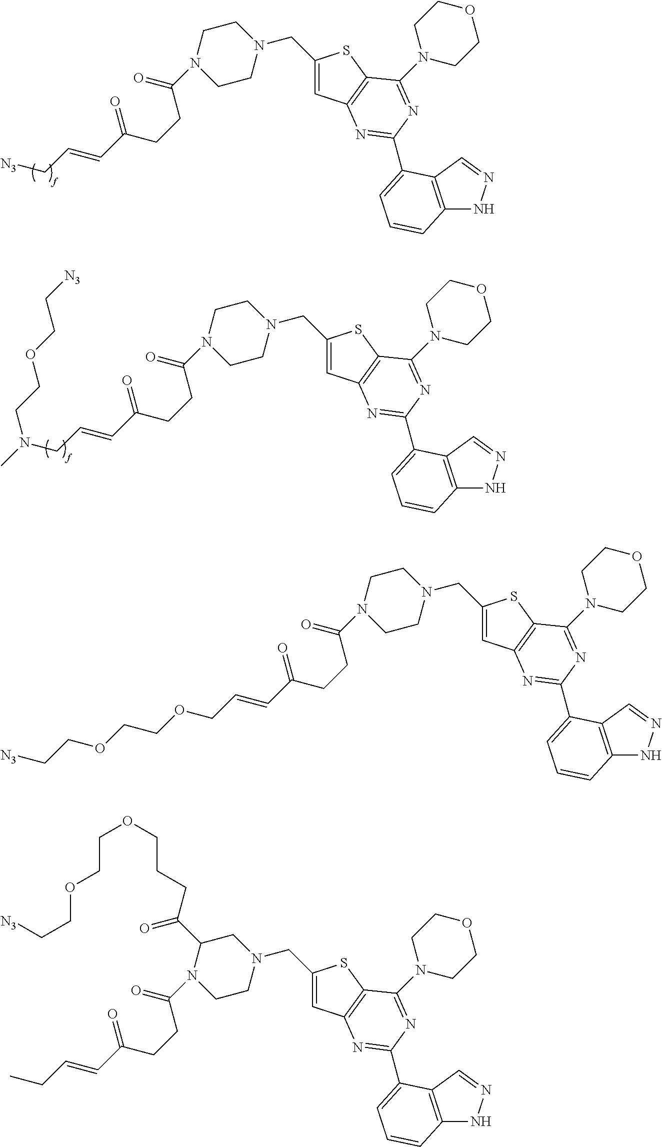

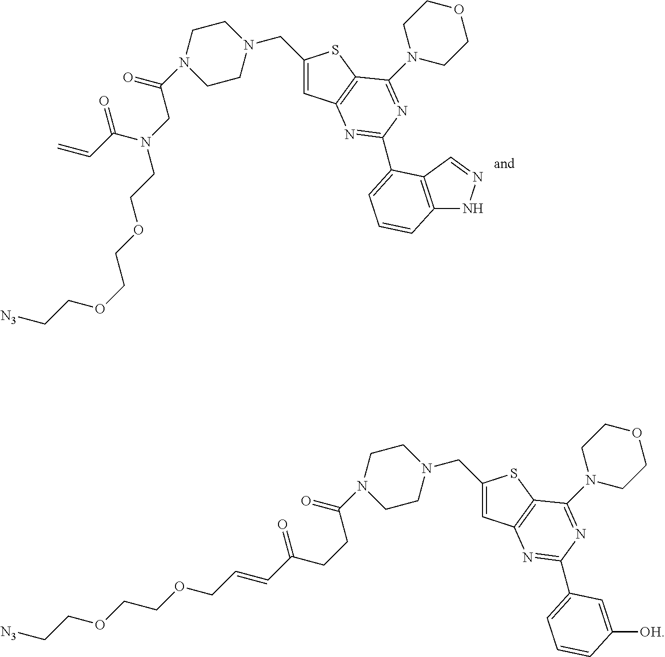

In some embodiments, the click ready -T^{p}-R^{p} moiety is of formula:

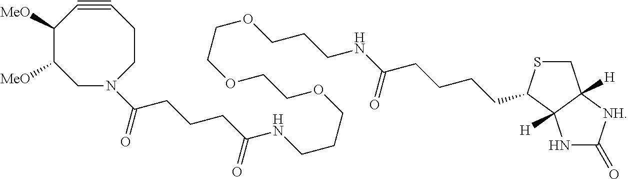

An exemplary reaction, including the use of the cyclooctyne (see Sletten and Bertozzi, Org. Lett. 10: 3097-3099 (2008)), in which a click ready inhibitor moiety and a click ready -T^{p}-R^{p} moiety are joined through a [3+2]-cycloaddition is as follows:

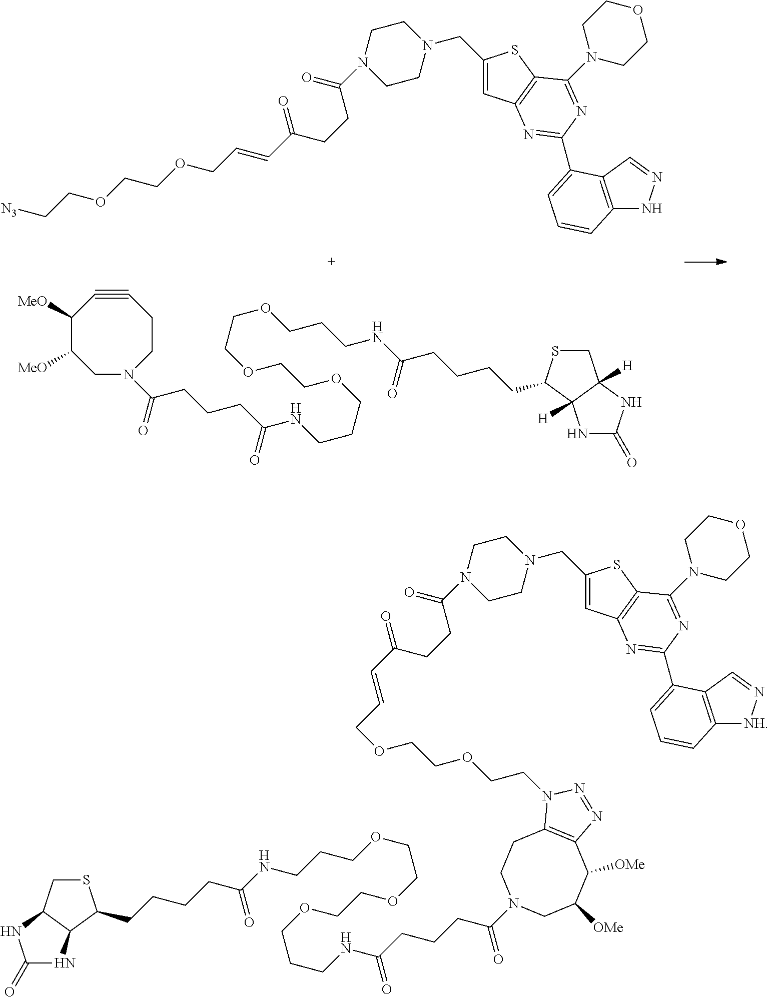

In some embodiments, the detectable moiety, R^{p}, is selected from a label, a dye, a photocrosslinker, a cytotoxic compound, a drug, an affinity label, a photoaffinity label, a reactive compound, an antibody or antibody fragment, a biomaterial, a nanoparticle, a spin label, a fluorophore, a metal-containing moiety, a radioactive moiety, quantum dot(s), a novel functional group, a group that covalently or noncovalently interacts with other molecules, a photocaged moiety, an actinic radiation excitable moiety, a ligand, a photoisomerizable moiety, biotin, a biotin analog (e.g., biotin sulfoxide), a moiety incorporating a heavy atom, a chemically cleavable group, a photocleavable group, a redox-active agent, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, or a combination thereof.

In some embodiments, R^{p} is biotin or an analog thereof. In certain embodiments, R^{p} is biotin. In certain other embodiments, R^{p} is biotin sulfoxide.

In another embodiment, R^{p} is a fluorophore. In a further embodiment, the fluorophore is selected from Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 493/503, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X, 5(6)-Carboxyfluorescein, 2,7-Dichlorofluorescein, N,N-Bis(2,4,6-trimethylphenyl)-3,4:9,10-perylenebis(dicarboximide, HPTS, Ethyl Eosin, DY-490XL MegaStokes, DY-485XL MegaStokes, Adirondack Green 520, ATTO 465, ATTO 488, ATTO 495, YOYO-1,5-FAM, BCECF, dichlorofluorescein, rhodamine 110, rhodamine 123, YO-PRO-1, SYTOX Green, Sodium Green, SYBR Green I, Alexa Fluor 500, FITC, Fluo-3, Fluo-4, fluoro-emerald, YoYo-1 ssDNA, YoYo-1 dsDNA, YoYo-1, SYTO RNASelect, Diversa Green-FP, Dragon Green, EvaGreen, Surf Green EX, Spectrum Green, NeuroTrace 500525, NBD-X, MitoTracker Green FM, LysoTracker Green DND-26, CBQCA, PA-GFP (post-activation), WEGFP (post-activation), FLASH-CCXXCC, Azami Green monomeric, Azami Green, green fluorescent protein (GFP), EGFP (Campbell Tsien 2003), EGFP (Patterson 2001), Kaede Green, 7-Benzylamino-4-Nitrobenz-2-Oxa-1,3-Diazole, Bexl, Doxorubicin, Lumio Green, or SuperGlo GFP.

As described generally above, a provided probe compound comprises a tethering moiety, -T^{p}-, that attaches the irreversible inhibitor to the detectable moiety. As used herein, the term “tether” or “tethering moiety” refers to any bivalent chemical spacer including, but not limited to, a covalent bond, a polymer, a water soluble polymer, optionally substituted alkyl, optionally substituted heteroalkyl, optionally substituted heterocycloalkyl, optionally substituted cycloalkyl, optionally substituted heterocyclyl, optionally substituted heterocycloalkylalkyl, optionally substituted heterocycloalkylalkenyl, optionally substituted aryl, optionally substituted heteroaryl, optionally substituted heterocycloalkylalkenylalkyl, an optionally substituted amide moiety, an ether moiety, an ketone moiety, an ester moiety, an optionally substituted carbamate moiety, an optionally substituted hydrazone moiety, an optionally substituted hydrazine moiety, an optionally substituted oxime moiety, a disulfide moiety, an optionally substituted imine moiety, an optionally substituted sulfonamide moiety, a sulfone moiety, a sulfoxide moiety, a thioether moiety, or any combination thereof.

In some embodiments, the tethering moiety, -T^{p}-, is selected from a covalent bond, a polymer, a water soluble polymer, optionally substituted alkyl, optionally substituted heteroalkyl, optionally substituted heterocycloalkyl, optionally substituted cycloalkyl, optionally substituted heterocycloalkylalkyl, optionally substituted heterocycloalkylalkenyl, optionally substituted aryl, optionally substituted heteroaryl, and optionally substituted heterocycloalkylalkenylalkyl. In some embodiments, the tethering moiety is an optionally substituted heterocycle. In other embodiments, the heterocycle is selected from aziridine, oxirane, episulfide, azetidine, oxetane, pyrroline, tetrahydrofuran, tetrahydrothiophene, pyrrolidine, pyrazole, pyrrole, imidazole, triazole, tetrazole, oxazole, isoxazole, oxirene, thiazole, isothiazole, dithiolane, furan, thiophene, piperidine, tetrahydropyran, thiane, pyridine, pyran, thiapyrane, pyridazine, pyrimidine, pyrazine, piperazine, oxazine, thiazine, dithiane, and dioxane. In some embodiments, the heterocycle is piperazine. In further embodiments, the tethering moiety is optionally substituted. In other embodiments, the water soluble polymer is a PEG group.

In other embodiments, the tethering moiety provides sufficient spatial separation between the detectable moiety and the kinase inhibitor moiety. In further embodiments, the tethering moiety is stable. In yet a further embodiment, the tethering moiety does not substantially affect the response of the detectable moiety. In other embodiments, the tethering moiety provides chemical stability to the probe compound. In further embodiments, the tethering moiety provides sufficient solubility to the probe compound.

In some embodiments, a tethering moiety, -T^{p}-, such as a water soluble polymer is coupled at one end to a provided irreversible inhibitor and to a detectable moiety, R^{p}, at the other end. In other embodiments, a water soluble polymer is coupled via a functional group or substituent of the provided irreversible inhibitor. In further embodiments, a water soluble polymer is coupled via a functional group or substituent of the reporter moiety.

In some embodiments, examples of hydrophilic polymers, for use in tethering moiety -T^{p}-, include, but are not limited to: polyalkyl ethers and alkoxy-capped analogs thereof (e.g., polyoxyethylene glycol, polyoxyethylene/propylene glycol, and methoxy or ethoxy-capped analogs thereof, polyoxyethylene glycol, the latter is also known as polyethylene glycol or PEG); polyvinylpyrrolidones; polyvinylalkyl ethers; polyoxazolines, polyalkyl oxazolines and polyhydroxyalkyl oxazolines; polyacrylamides, polyalkyl acrylamides, and polyhydroxyalkyl acrylamides (e.g., polyhydroxypropylmethacrylamide and derivatives thereof); polyhydroxyalkyl acrylates; polysialic acids and analogs thereof, hydrophilic peptide sequences; polysaccharides and their derivatives, including dextran and dextran derivatives, e.g., carboxymethyldextran, dextran sulfates, aminodextran; cellulose and its derivatives, e.g., carboxymethyl cellulose, hydroxyalkyl celluloses; chitin and its derivatives, e.g., chitosan, succinyl chitosan, carboxymethylchitin, carboxymethylchitosan; hyaluronic acid and its derivatives; starches; alginates; chondroitin sulfate; albumin; pullulan and carboxymethyl pullulan; polyaminoacids and derivatives thereof, e.g., polyglutamic acids, polylysines, polyaspartic acids, polyaspartamides; maleic anhydride copolymers such as: styrene maleic anhydride copolymer, divinylethyl ether maleic anhydride copolymer; polyvinyl alcohols; copolymers thereof, terpolymers thereof, mixtures thereof, and derivatives of the foregoing. In other embodiments, a water soluble polymer is any structural form including but not limited to linear, forked or branched. In further embodiments, multifunctional polymer derivatives include, but are not limited to, linear polymers having two termini, each terminus being bonded to a functional group which is the same or different.

In some embodiments, a water polymer comprises a poly(ethylene glycol) moiety. In further embodiments, the molecular weight of the polymer is of a wide range, including but not limited to, between about 100 Da and about 100,000 Da or more. In yet further embodiments, the molecular weight of the polymer is between about 100 Da and about 100,000 Da, including but not limited to, about 100,000 Da, about 95,000 Da, about 90,000 Da, about 85,000 Da, about 80,000 Da, about 75,000 Da, about 70,000 Da, about 65,000 Da, about 60,000 Da, about 55,000 Da, about 50,000 Da, about 45,000 Da, about 40,000 Da, about 35,000 Da, 30,000 Da, about 25,000 Da, about 20,000 Da, about 15,000 Da, about 10,000 Da, about 9,000 Da, about 8,000 Da, about 7,000 Da, about 6,000 Da, about 5,000 Da, about 4,000 Da, about 3,000 Da, about 2,000 Da, about 1,000 Da, about 900 Da, about 800 Da, about 700 Da, about 600 Da, about 500 Da, about 400 Da, about 300 Da, about 200 Da, and about 100 Da. In some embodiments, the molecular weight of the polymer is between about 100 Da and 50,000 Da. In some embodiments, the molecular weight of the polymer is between about 100 Da and 40,000 Da. In some embodiments, the molecular weight of the polymer is between about 1,000 Da and 40,000 Da. In some embodiments, the molecular weight of the polymer is between about 5,000 Da and 40,000 Da. In some embodiments, the molecular weight of the polymer is between about 10,000 Da and 40,000 Da. In some embodiments, the poly(ethylene glycol) molecule is a branched polymer. In further embodiments, the molecular weight of the branched chain PEG is between about 1,000 Da and about 100,000 Da, including but not limited to, about 100,000 Da, about 95,000 Da, about 90,000 Da, about 85,000 Da, about 80,000 Da, about 75,000 Da, about 70,000 Da, about 65,000 Da, about 60,000 Da, about 55,000 Da, about 50,000 Da, about 45,000 Da, about 40,000 Da, about 35,000 Da, about 30,000 Da, about 25,000 Da, about 20,000 Da, about 15,000 Da, about 10,000 Da, about 9,000 Da, about 8,000 Da, about 7,000 Da, about 6,000 Da, about 5,000 Da, about 4,000 Da, about 3,000 Da, about 2,000 Da, and about 1,000 Da. In some embodiments, the molecular weight of a branched chain PEG is between about 1,000 Da and about 50,000 Da. In some embodiments, the molecular weight of a branched chain PEG is between about 1,000 Da and about 40,000 Da. In some embodiments, the molecular weight of a branched chain PEG is between about 5,000 Da and about 40,000 Da. In some embodiments, the molecular weight of a branched chain PEG is between about 5,000 Da and about 20,000 Da. The foregoing list for substantially water soluble backbones is by no means exhaustive and is merely illustrative, and in some embodiments, polymeric materials having the qualities described above are suitable for use in methods and compositions described herein.

One of ordinary skill in the art will appreciate that when -T^{p}-R^{p} is attached to a compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e via the R¹ warhead group, then the resulting tethering moiety comprises the R¹ warhead group. As used herein, the phrase “comprises a warhead group” means that the tethering moiety formed by —R^{1′}-T^{p}- of formula XIII, XIV, XIV-a, XIV-b, XIV-c, XIV-d, XIV-e, XIV-f, XIV-g, XIV-h, XV, XVI, XVII-a, XVII-b, XVIII-a, XVIII-b, XIX, XX, XXI, XXII, XXIII, XXIV, XXIV-a, XXIV-b, XXIV-c, XXIV-d, or XXIV-e is either substituted with a warhead group or has such a warhead group incorporated within the tethering moiety. For example, the tethering moiety formed by —R^{1′}-T^{p}- may be substituted with an -L-Y warhead group, wherein such groups are as described herein. Alternatively, the tethering moiety formed by —R^{1′}-T^{p}- has the appropriate features of a warhead group incorporated within the tethering moiety. For example, the tethering moiety formed by —R^{1′}-T^{p}- may include one or more units of unsaturation and optional substituents and/or heteroatoms which, in combination, result in a moiety that is capable of covalently modifying a kinase in accordance with the present invention. Such —R¹-T^{p}- tethering moiety are depicted below.

In some embodiments, a methylene unit of an —R^{1′}-T^{p}-tethering moiety is replaced by a bivalent -L-Y′-moiety to provide a compound of formula XIII-i, XIV-i, XIV-a-i, XIV-b-i, XIV-c-i, XIV-d-i, XIV-e-i, XIV-f-i, XIV-g-i, XIV-h-i, XV-i, XVI-i, XVII-a-i, XVII-b-i, XVIII-a-i, XVIII-b-i, XIX-i, XX-i, XXI-i, XXII-i, XXIII-i, XXIV-i, XXIV-a-i, XXIV-b-i, XXIV-c-i, XXIV-d-i, or XXIV-e-i:

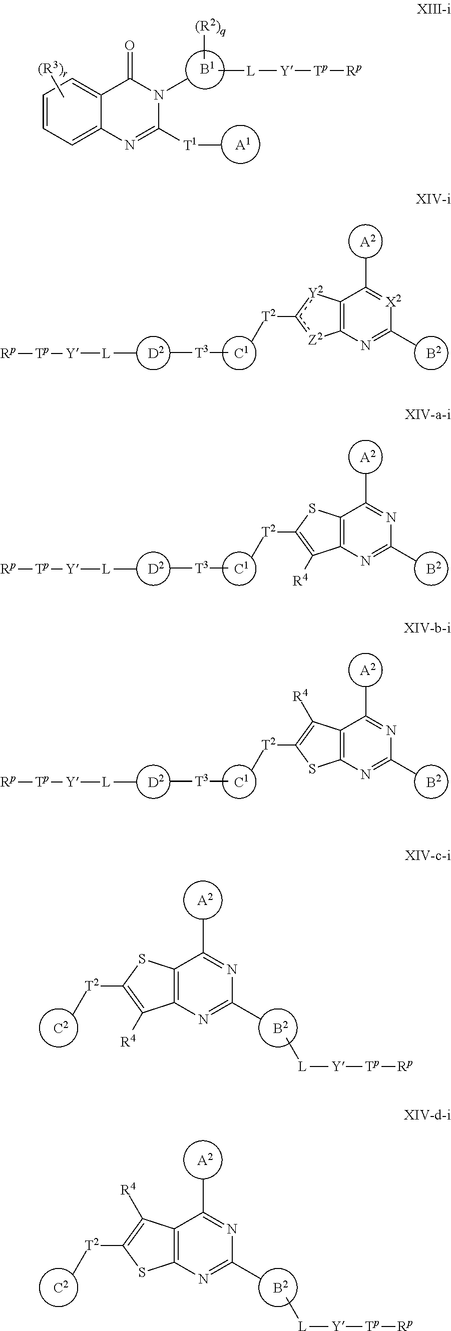

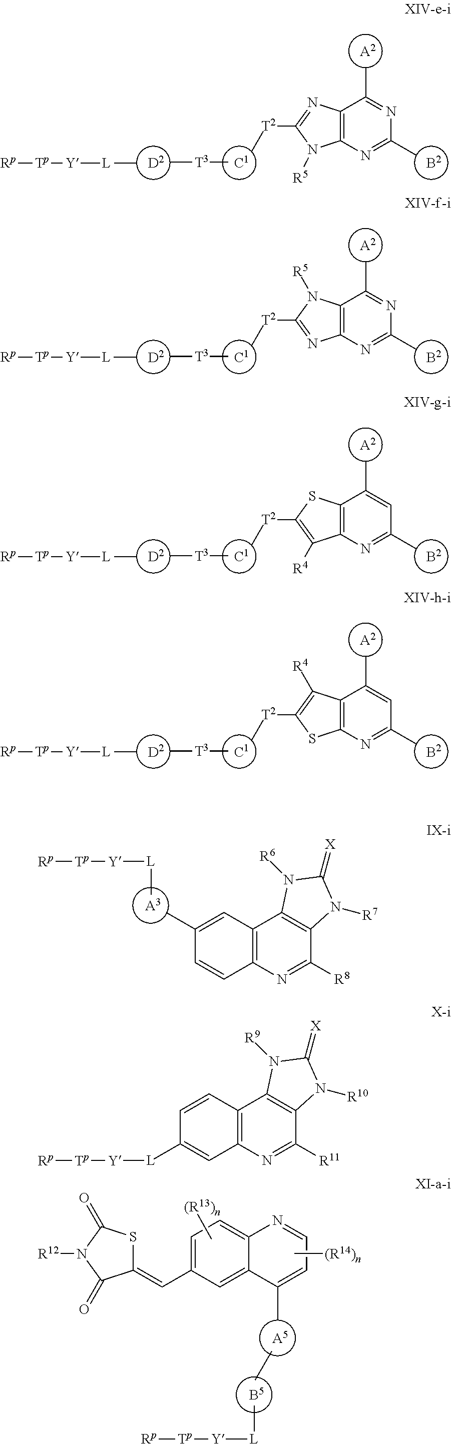

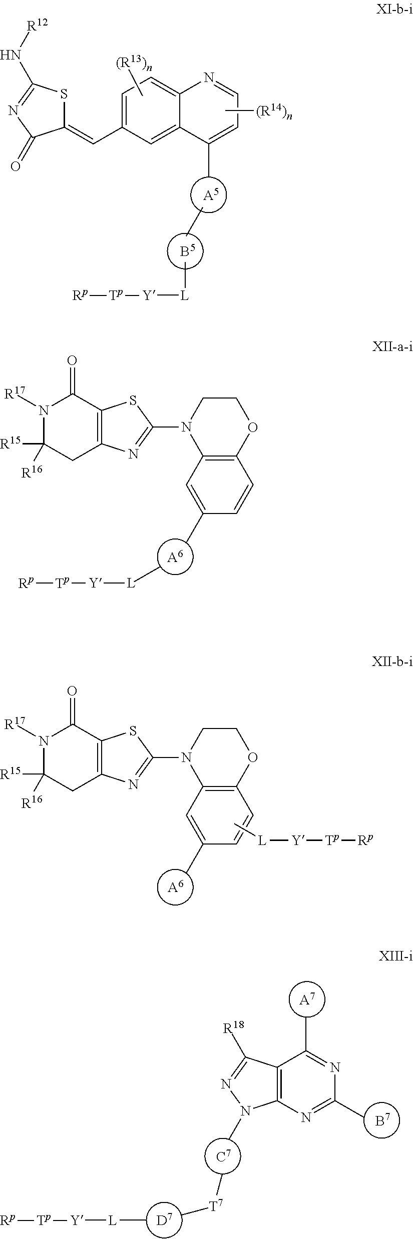

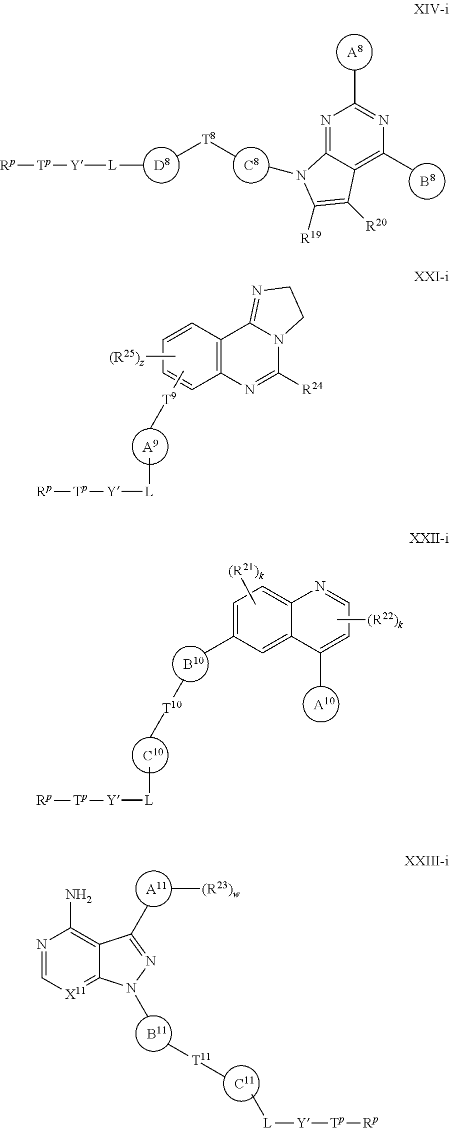

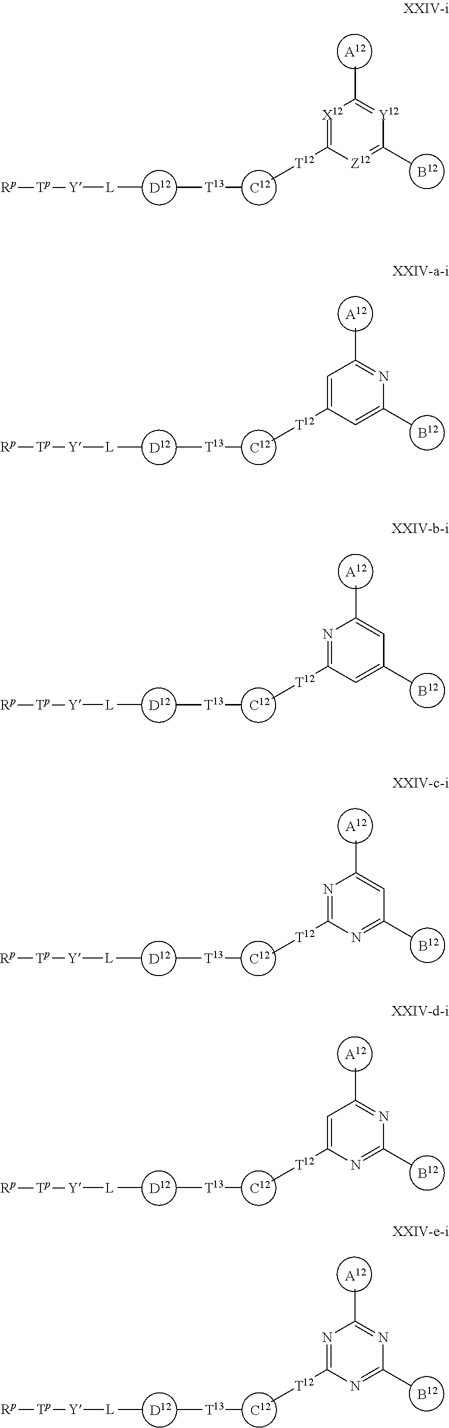

wherein each variable is as defined above for formulae I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, and XII-e and described in classes and subclasses herein, and Y′ is a bivalent version of the Y group defined above and described in classes and subclasses herein.

In some embodiments, a methylene unit of an —R^{1′}-T-tethering moiety is replaced by an -L(Y)— moiety to provide a compound of formula XIII-ii, XIV-ii, XIV-a-ii, XIV-b-ii, XIV-c-ii, XIV-d-ii, XIV-e-ii, XIV-f-ii, XIV-g-ii, XIV-h-ii, XV-ii, XVI-ii, XVII-a-ii, XVII-b-ii, XVIII-a-ii, XVIII-b-ii, XIX-ii, XX-ii, XXI-ii, XXII-ii, XXIII-ii, XXIV-ii, XXIV-a-ii, XXIV-b-ii, XXIV-c-ii, XXIV-d-ii, or XXIV-e-ii:

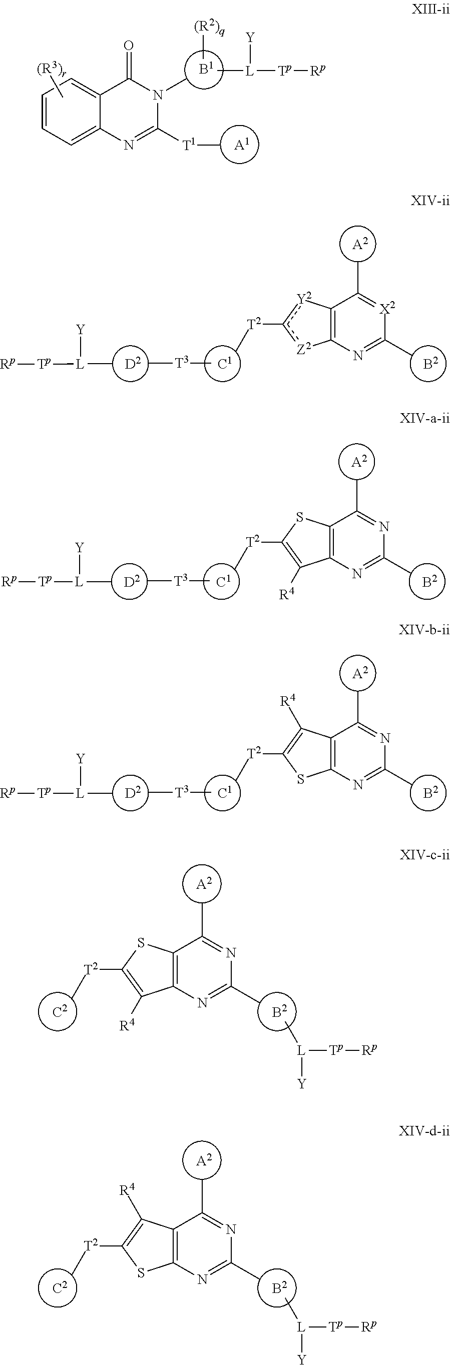

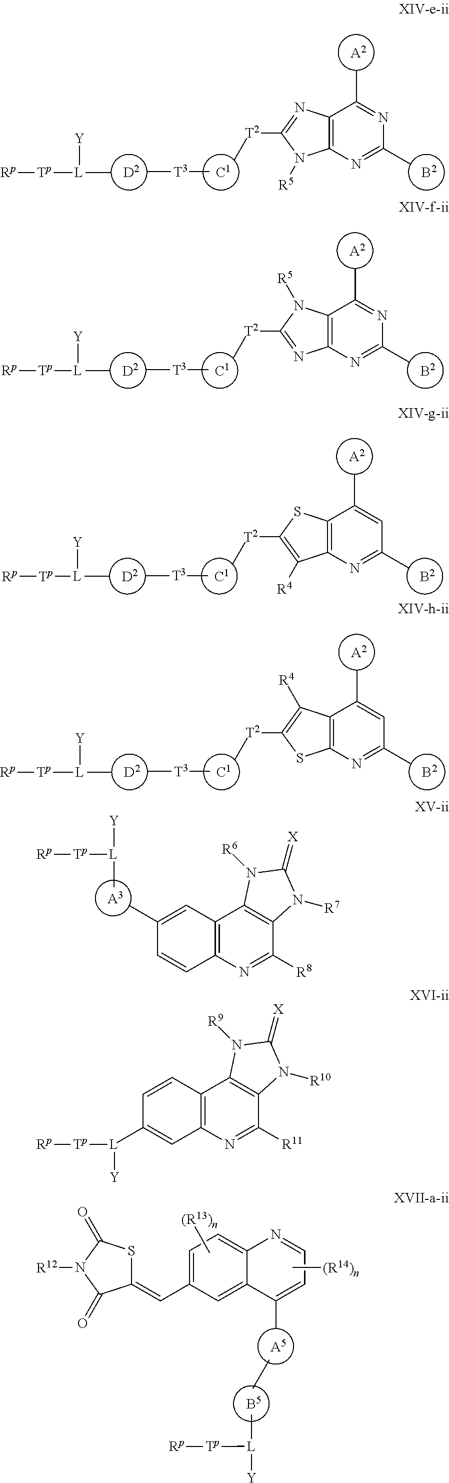

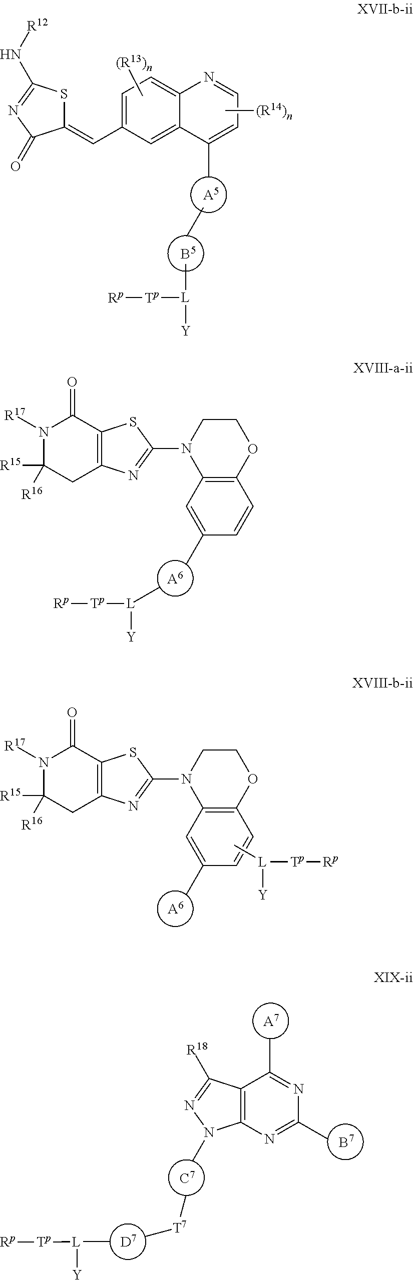

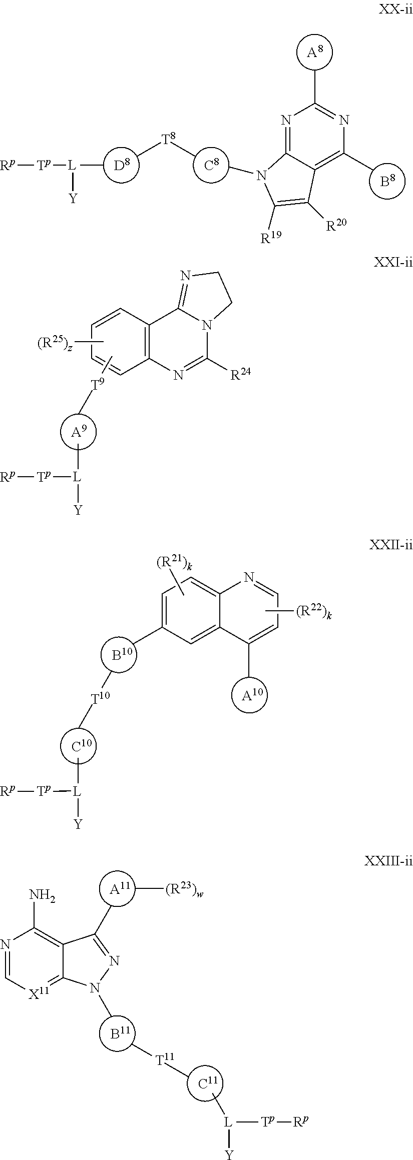

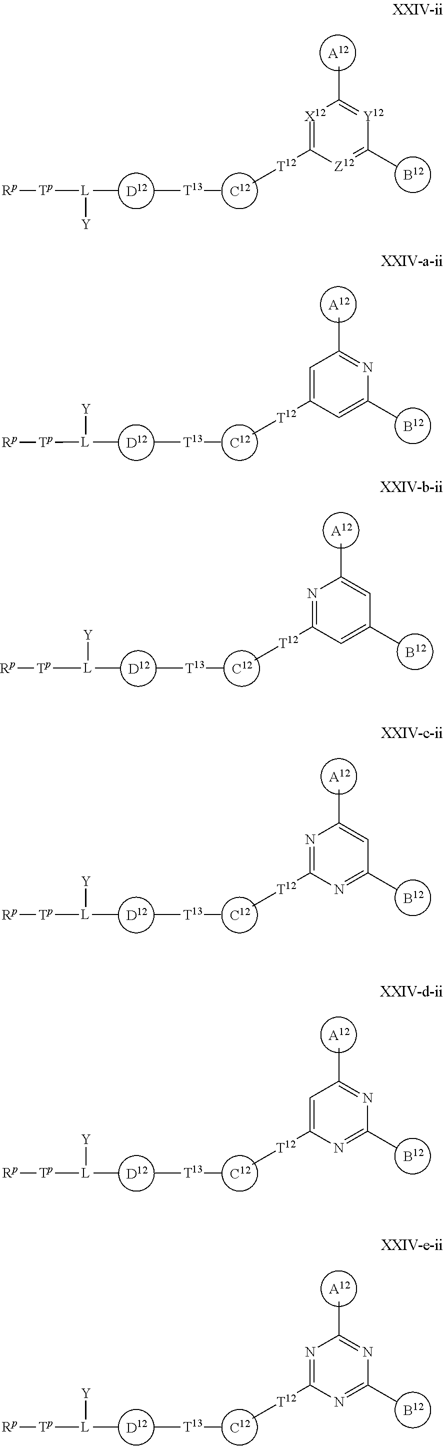

wherein each variable is as defined above for formulae I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, and XII-e and described in classes and subclasses herein.

In some embodiments, a tethering moiety is substituted with an L-Y moiety to provide a compound of formula XIII-iii, XIV-iii, XIV-a-iii, XIV-b-iii, XIV-c-iii, XIV-d-iii, XIV-e-iii, XIV-f-iii, XIV-g-iii, XIV-h-iii, XV-iii, XVI-iii, XVII-a-iii, XVII-b-iii, XVIII-a-iii, XVIII-b-iii, XIX-iii, XX-iii, XXI-iii, XXII-iii, XXIII-iii, XXIV-iii, XXIV-a-iii, XXIV-b-iii, XXIV-c-iii, XXIV-d-iii, or XXIV-e-iii:

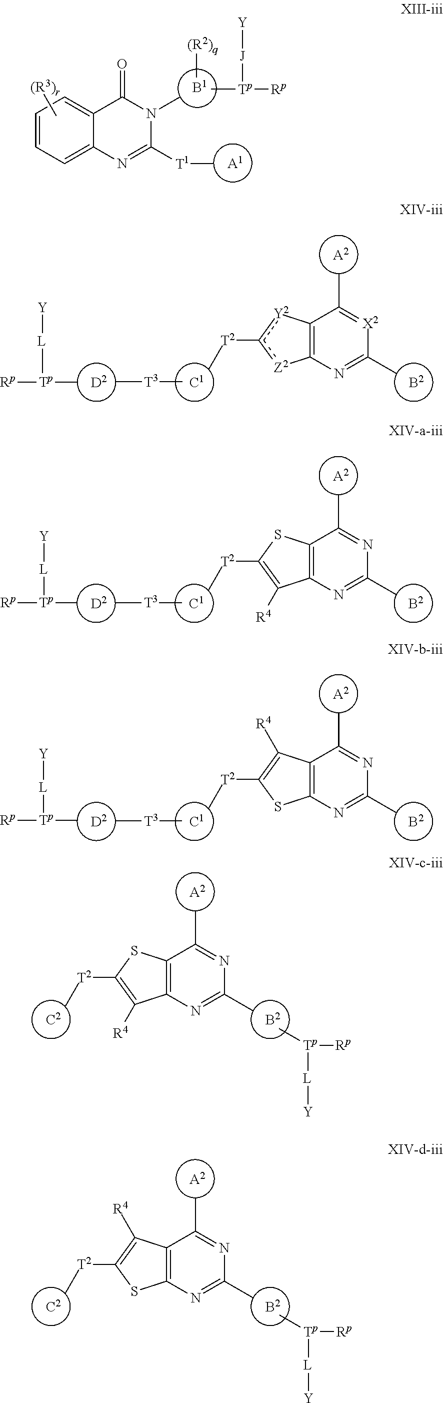

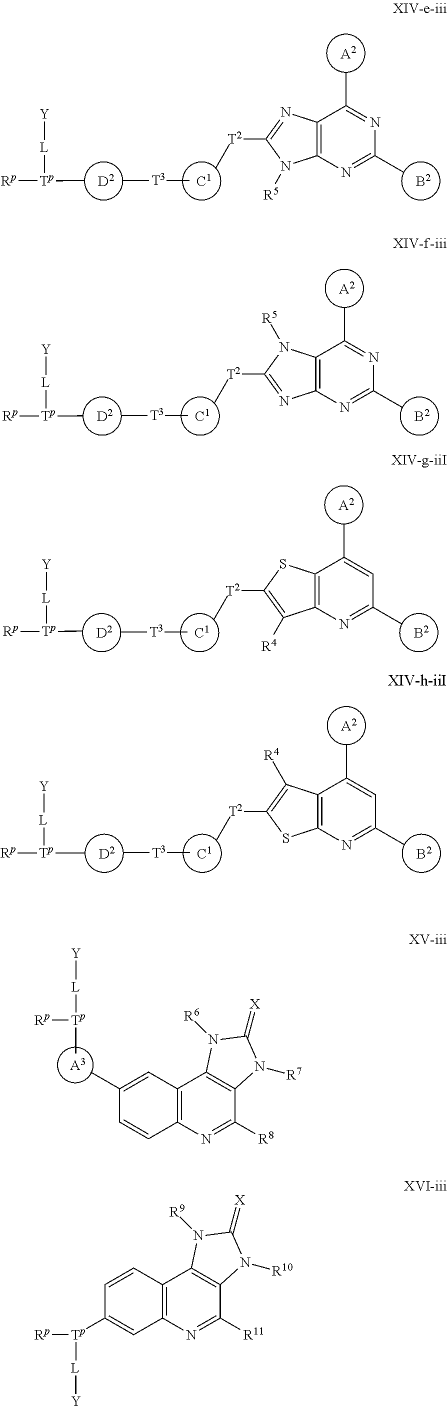

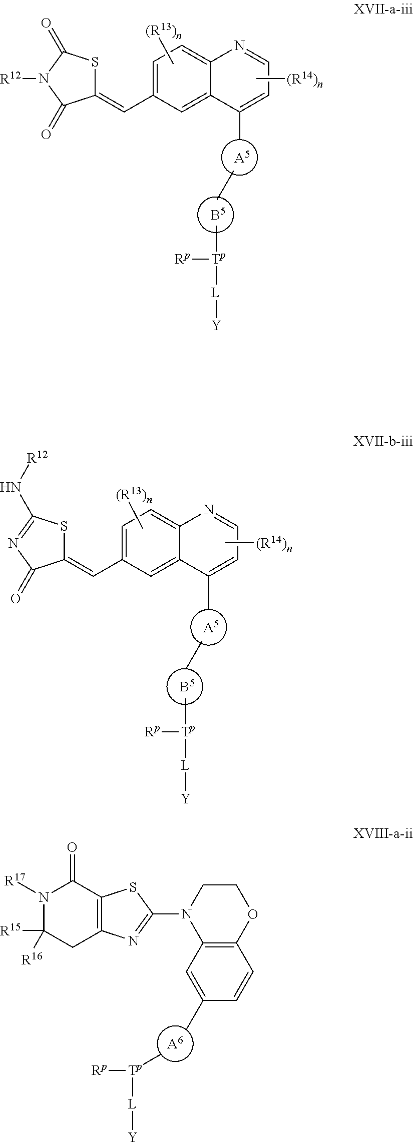

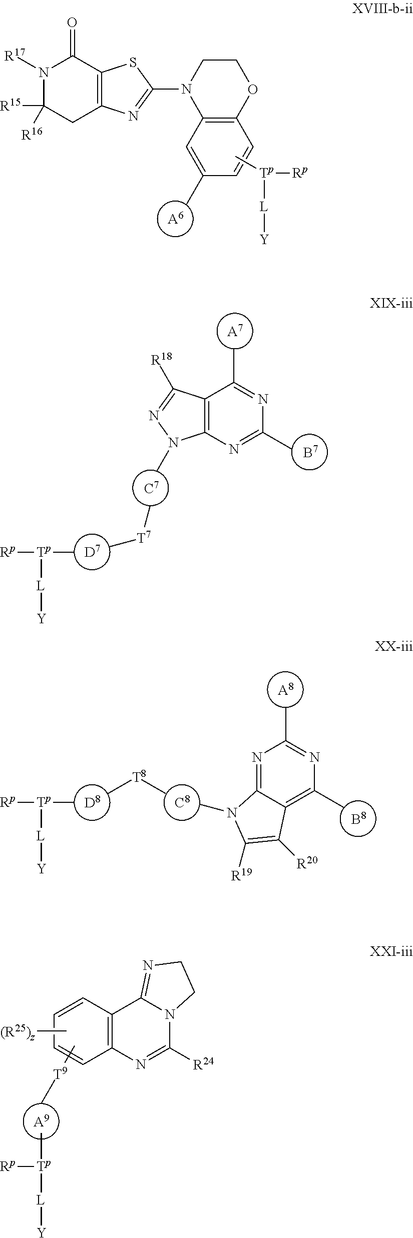

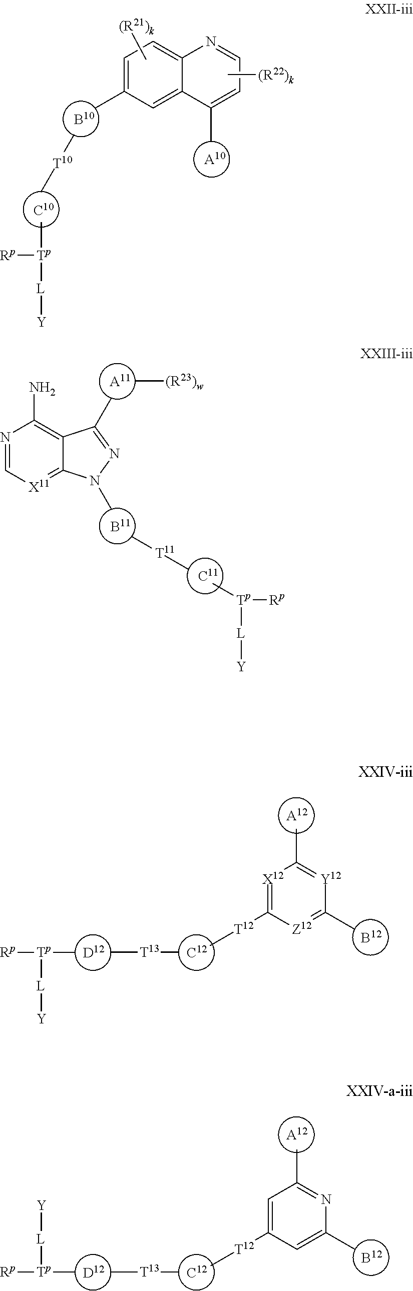

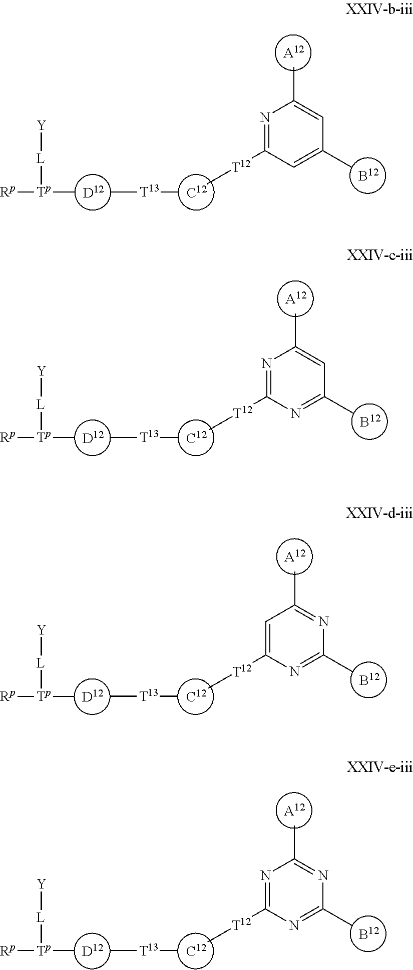

wherein each variable is as defined above for formulae I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, and XII-e and described in classes and subclasses herein.

In certain embodiments, the tethering moiety, -T^{p}-, has one of the following structures:

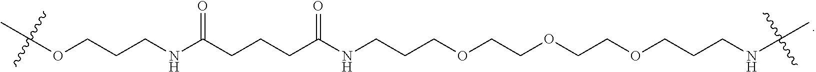

In some embodiments, the tethering moiety, -T^{p}-, has the following structure:

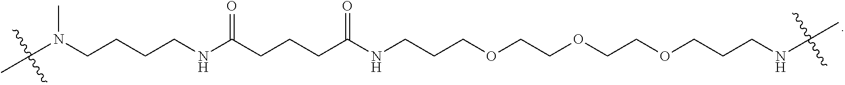

In other embodiments, the tethering moiety, -T^{p}-, has the following structure:

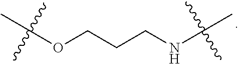

In certain other embodiments, the tethering moiety, -T^{p}-, has the following structure:

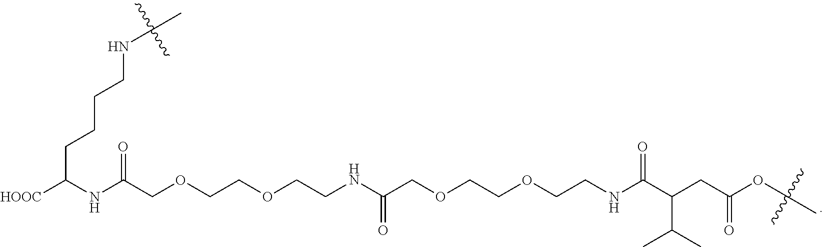

In yet other embodiments, the tethering moiety, -T^{p}-, has the following structure:

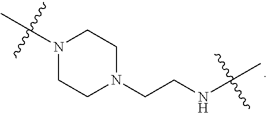

In some embodiments, the tethering moiety, -T^{p}-, has the following structure:

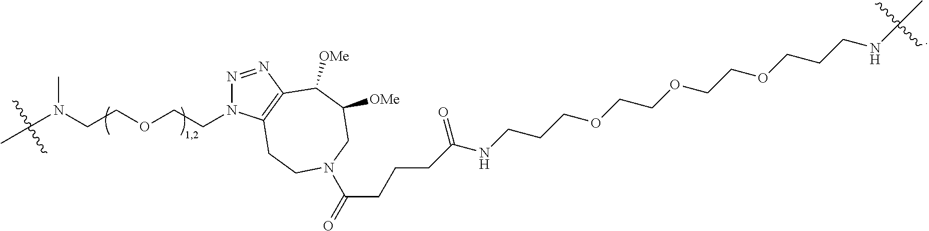

In some embodiments, -T^{p}-R^{p} is of the following structure:

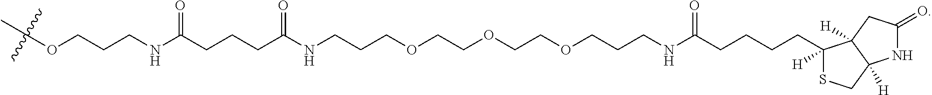

In other embodiments, -T^{p}-R^{p} is of the following structure:

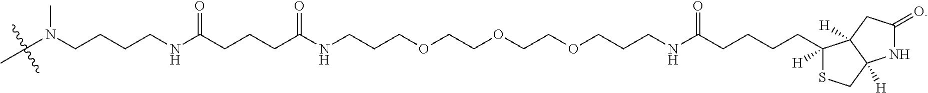

In certain embodiments, -T^{p}-R^{p} is of the following structure:

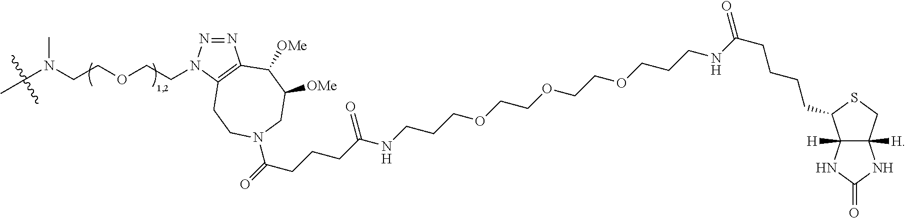

In some embodiments, a probe compound of formula XIII, XIV, XIV-a, XIV-b, XIV-c, XIV-d, XIV-e, XIV-f, XIV-g, XIV-h, XV, XVI, XVII-a, XVII-b, XVIII-a, XVIII-b, XIX, XX, XXI, XXII, XXIII, XXIV, XXIV-a, XXIV-b, XXIV-c, XXIV-d, or XXIV-e is derived from any compound of Tables 5-17.

In certain embodiments, the probe compound is one of the following structures:

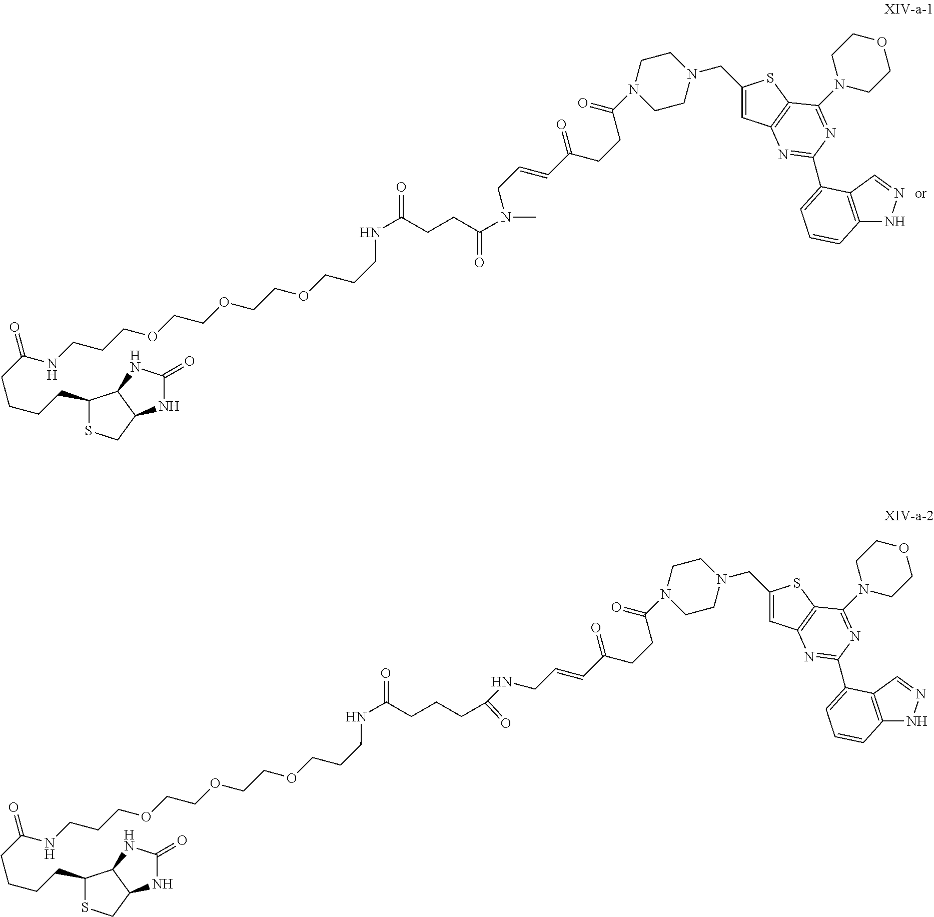

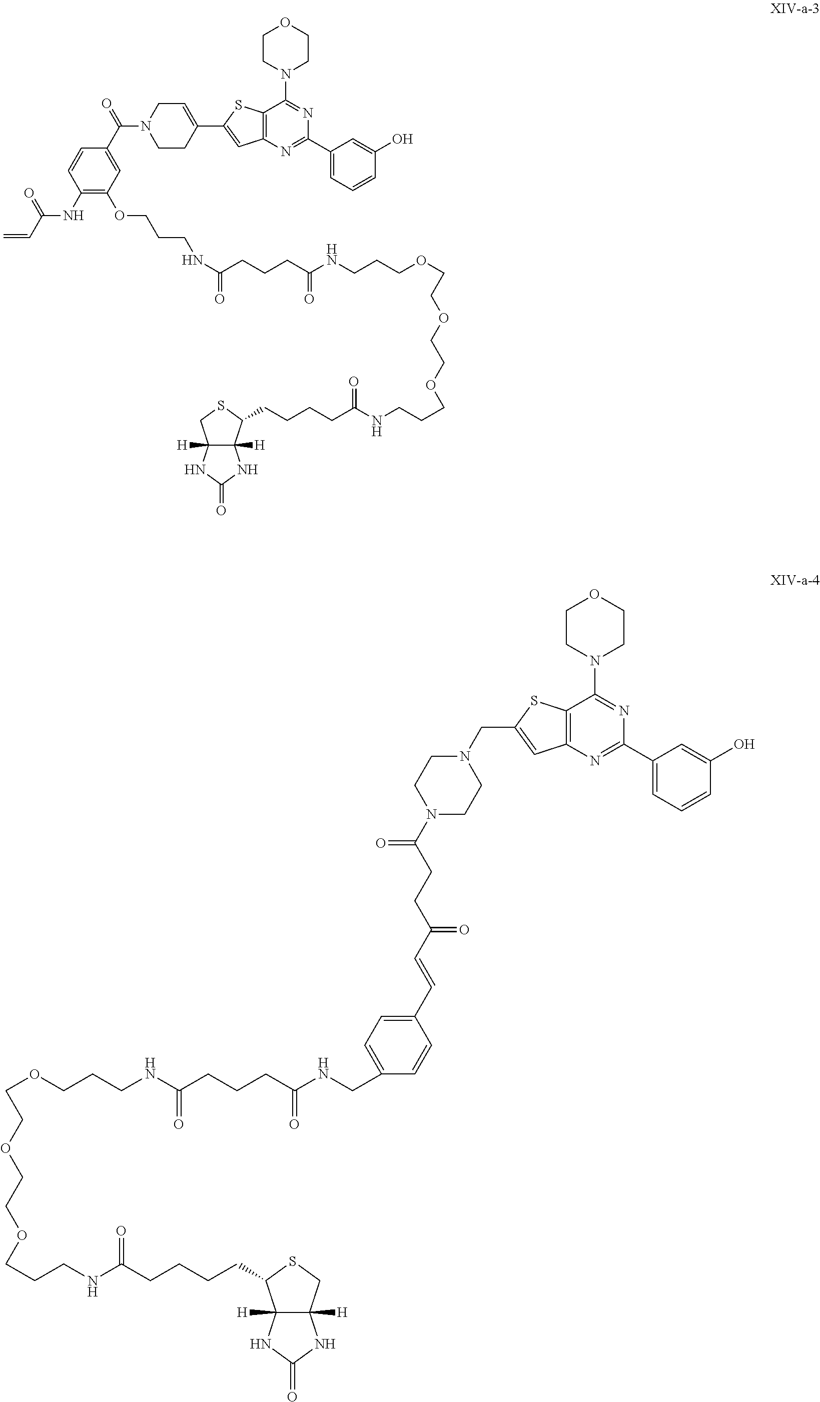

It will be appreciated that many -T^{p}-R^{p} reagents are commercially available. For example, numerous biotinylating reagents are available from, e.g., Thermo Scientific having varying tether lengths. Such reagents include NHS-PEG₄-Biotin and NHS-PEG₁₂-Biotin.

In some embodiments, analogous probe structures to the ones exemplified above are prepared using click-ready inhibitor moieties and click-ready -T^{p}-R^{p} moieties, as described herein.

In some embodiments, a provided probe compound covalently modifies a phosphorylated conformation of a kinase. In one aspect, the phosphorylated conformation of the kinase is either an active or inactive form of the kinase. In certain embodiments, the phosphorylated conformation of the kinase is an active form of said kinase. In certain embodiments, the probe compound is cell permeable.

In some embodiments, the present invention provides a method for determining occupancy of a kinase by a provided irreversible inhibitor (i.e., a compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e) in a patient, comprising providing one or more tissues, cell types, or a lysate thereof, obtained from a patient administered at least one dose of a compound of said irreversible inhibitor, contacting said tissue, cell type or lysate thereof with a probe compound (i.e., a compound of formula XIII, XIV, XIV-a, XIV-b, XIV-c, XIV-d, XIV-e, XIV-f, XIV-g, XIV-h, XV, XVI, XVII-a, XVII-b, XVIII-a, XVIII-b, XIX, XX, XXI, XXII, XXIII, XXIV, XXIV-a, XXIV-b, XXIV-c, XXIV-d, or XXIV-e) to covalent modify at least one kinase present in said lysate, and measuring the amount of said kinase covalently modified by the probe compound to determine occupancy of said kinase by said compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e as compared to occupancy of said kinase by said probe compound. In certain embodiments, the method further comprises the step of adjusting the dose of the compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e to increase occupancy of the kinase. In certain other embodiments, the method further comprises the step of adjusting the dose of the compound of formula I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e to decrease occupancy of the kinase.

As used herein, the terms “occupancy” or “occupy” refer to the extent to which a kinase is modified by a provided covalent inhibitor compound. One of ordinary skill in the art would appreciate that it is desirable to administer the lowest dose possible to achieve the desired efficacious occupancy of the kinase.

In some embodiments, the kinase to be modified is PI3K. In certain embodiments, the kinase to be modified is PI3K-α. In certain embodiments, the kinase to be modified is PI3K-γ. In some embodiments, the kinase to be modified is PI3K-β or PI3K-δ. In other embodiments, the kinase to be modified is mTOR, DNA-PK, ATM kinase, or PI4KA.

In some embodiments, the probe compound comprises the irreversible inhibitor for which occupancy is being determined.

In some embodiments, the present invention provides a method for assessing the efficacy of a provided irreversible inhibitor in a mammal, comprising administering a provided irreversible inhibitor to the mammal, administering a provided probe compound to tissues or cells isolated from the mammal, or a lysate thereof, measuring the activity of the detectable moiety of the probe compound, and comparing the activity of the detectable moiety to a standard.

In other embodiments, the present invention provides a method for assessing the pharmacodynamics of a provided irreversible inhibitor in a mammal, comprising administering a provided irreversible inhibitor to the mammal, administering a probe compound presented herein to one or more cell types, or a lysate thereof, isolated from the mammal, and measuring the activity of the detectable moiety of the probe compound at different time points following the administration of the inhibitor.

In yet other embodiments, the present invention provides a method for in vitro labeling of a protein kinase comprising contacting said protein kinase with a probe compound described herein. In one embodiment, the contacting step comprises incubating the protein kinase with a probe compound presented herein.

In certain embodiments, the present invention provides a method for in vitro labeling of a protein kinase comprising contacting one or more cells or tissues, or a lysate thereof, expressing the protein kinase with a probe compound described herein.

In certain other embodiments, the present invention provides a method for detecting a labeled protein kinase comprising separating proteins, the proteins comprising a protein kinase labeled by probe compound described herein, by electrophoresis and detecting the probe compound by fluorescence.

In some embodiments, the present invention provides a method for assessing the pharmacodynamics of a provided irreversible inhibitor in vitro, comprising incubating the provided irreversible inhibitor with the target protein kinase, adding the probe compound presented herein to the target protein kinase, and determining the amount of target modified by the probe compound.

In certain embodiments, the probe compound is detected by binding to avidin, streptavidin, neutravidin, or captavidin.

In some embodiments, the probe is detected by Western blot. In other embodiments, the probe is detected by ELISA. In certain embodiments, the probe is detected by flow cytometry.

In other embodiments, the present invention provides a method for probing the kinome with irreversible inhibitors comprising incubating one or more cell types, or a lysate thereof, with a biotinylated probe compound to generate proteins modified with a biotin moiety, digesting the proteins, capturing with avidin or an analog thereof, and performing multi-dimensional LC-MS-MS to identify protein kinases modified by the probe compound and the adduction sites of said kinases.

In certain embodiments, the present invention provides a method for measuring protein synthesis in cells comprising incubating cells with an irreversible inhibitor of the target protein, forming lysates of the cells at specific time points, and incubating said cell lysates with an inventive probe compound to measure the appearance of free protein over an extended period of time.

In other embodiments, the present invention provides a method for determining a dosing schedule in a mammal for maximizing occupancy of a target protein kinase comprising assaying a one or more cell types, or a lysate thereof, isolated from the mammal, (derived from, e.g., splenocytes, peripheral B cells, whole blood, lymph nodes, intestinal tissue, or other tissues) from a mammal administered a provided irreversible inhibitor of formula I I, II, II-a, II-b, II-c, II-d, II-e, II-f, II-g, II-h, III, IV, V-a, V-b, VI-a, VI-b, VII, VIII, IX, X, XI, XII, XII-a, XII-b, XII-c, XII-d, or XII-e, wherein the assaying step comprises contacting said one or more tissues, cell types, or a lysate thereof, with a provided probe compound and measuring the amount of protein kinase covalently modified by the probe compound.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

Compound numbers utilized in the Examples below correspond to compound numbers set forth in Tables 5-17, supra.

### Example 1

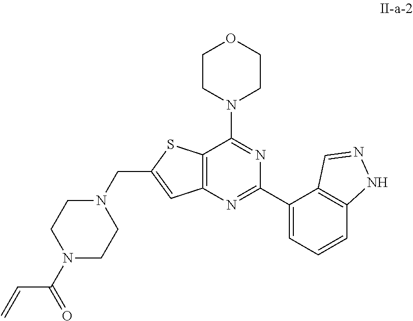

1-(4-((2-(1H-indazol-4-yl)-4-morpholinothieno[3,2-d]pyrimidin-6-yl)methyl)piperazin-1-yl)prop-2-en-1-one (II-a-2): The title compound was prepared according to the steps and intermediates as described below.

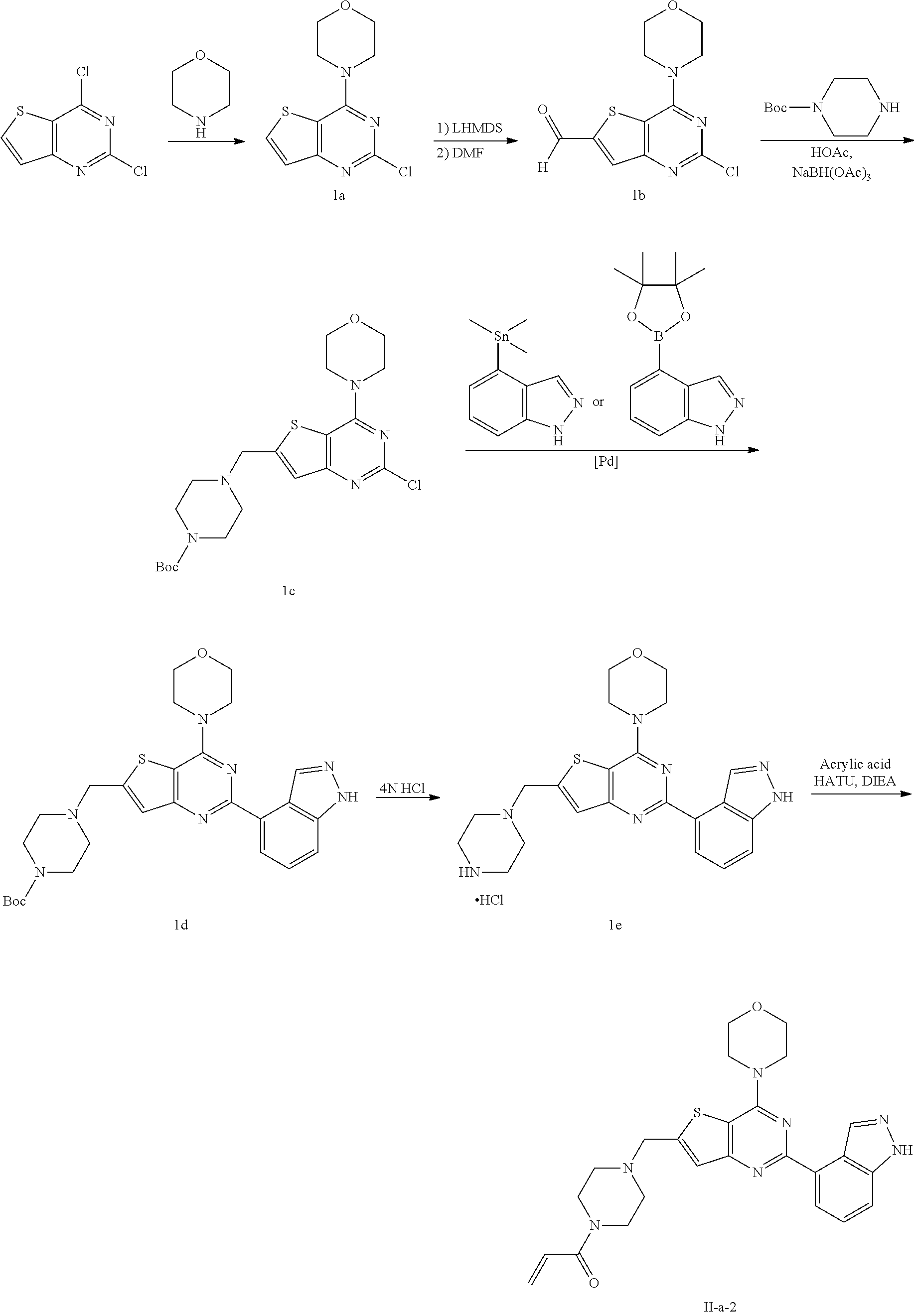

### Step 1a: 4-(2-chlorothieno[3,2-d]pyrimidin-4-yl)morpholine (Intermediate 1a)

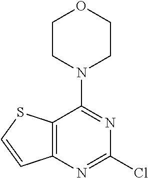

To a solution of 2,4-dichlorothieno[3,2-d]pyrimidine (2.0 g, 9.7 mmol) in 30 ml MeOH was added 1.9 ml morpholine. After stirring at room temperature for one hour, the reaction mixture was filtered; the solid was washed with water and methanol to provide 2.0 g of the title compound. MS m/z: 256.0, 258.1 (M+1). ¹H NMR (400 MHz, CDCl₃): δ: 7.78 (1H, d, J=5.48 Hz), 7.38 (1H, d, J=5.48 Hz), 4.02 (4H, t, J=4.80 Hz), 3.85 (4H, t, J=4.82 Hz).

### Step 1b: 2-chloro-4-morpholinothieno[3,2-d]pyrimidine-6-carbaldehyde (Intermediate 1b)

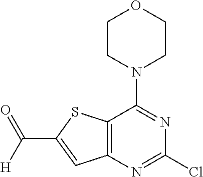

To a suspension of Intermediate 1a (1.02 g, 4.0 mmol) in 30 ml THF at −78° C. was added LiHMDS (1.0 N, 6.0 ml, 6.0 mmol) slowly. The reaction mixture was stirred at −78° C. for 1 h, DMF (0.5 ml) was added and reaction mixture was allowed to warm up to room temperature over 2 hours. The reaction was quenched with NH₄Cl aqueous solution and the THF was removed under vacuum. A 50-ml portion of EtOAc was added in and the mixture was washed with aqueous NaHCO₃ and brine. The organic layer was separated and was dried over Na₂SO₄. After removal of solvent, the crude product was subject to chromatography on silica gel (eluents: EtOAc/hexane). A total of 0.6 g of the title compound was obtained (60%). MS m/z: 284.2 (ES+, M+1).

### Step 1c: tert-butyl 4-((2-chloro-4-morpholinothieno[3,2-d]pyrimidin-6-yl)methyl)piperazine-1-carboxylate (Intermediate 1c)

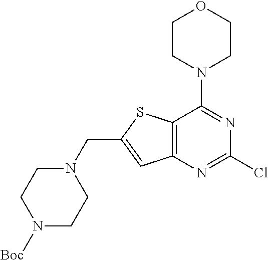

Intermediate 1b (0.40 g, 1.5 mmol), tert-butyl piperazine-1-carboxylate and 0.2 ml acetic acid were dissolved in 12 ml dichloroethane. The mixture was stirred at room temperature for 2 hours. NaBH(OAc)₃ (0.54 g, 2.5 mmol) was added to the reaction mixture and the resulting mixture was stirred at room temperature for 10 hours. A 20-ml of NaHCO₃ aqueous solution and 10 ml of DCM were added. The organic layer was separated and dried over Na₂SO₄. After removal of solvent, the crude product was subject to chromatography on silica gel (eluents: EtOAc/hexane 3:7). A total of 0.30 g of the title compound was obtained. MS m/z: 454.2 (ES+, M+1).

### Step 1d: tert-butyl 4-((2-(1H-indazol-4-yl)-4-morpholinothieno[3,2-d]pyrimidin-6-yl)methyl)piperazine-1-carboxylate (Intermediate 1d)

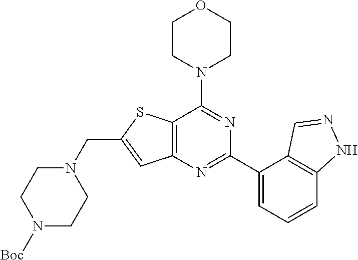

Intermediate 1c (0.14 g, 0.31 mmol), 4-(trimethylstannyl)-1H-indazole (0.10 g, 0.37 mmol) and tetrakis(triphenylphosphine)palladium (35 mg, 0.03 mmol) were dissolved in 5 ml toluene. The solution was degassed and flushed with N₂. The reaction mixture was heated to 135° C. for 40 hours in a sealed vial. The solvent was removed under vacuum and the residue was purified by chromatography on silica gel (eluents: EtOAc/hexane 5:5). A total of 0.10 g of the title compound was obtained. MS m/z: 536.1 (M+1).

Alternatively, Intermediate 1d can be prepared by using 4-(4,4,5,5-tetramethyl-1,3,2-dioxaborolan-2-yl)-1H-indazole instead of 4-(trimethylstannyl)-1H-indazole under standard Suzuki coupling conditions.

### Step 1e: 4-(2-(1H-indazol-4-yl)-6-(piperazin-1-ylmethyl)thieno[3,2-d]pyrimidin-4-yl)morpholine (Intermediate 1e)

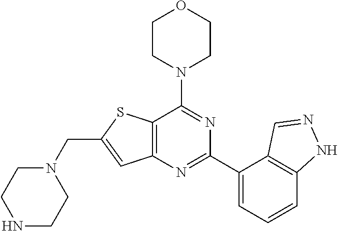

Intermediate 1d (100 mg, 0.18 mmol) was dissolved in 3 ml of 4N HCl in dixoxane and the reaction was stirred for 3 hours at room temperature. After removal of solvents, a 3-ml portion of DCM was poured in followed by evaporation to dryness. This process of DCM addition followed by evaporation was repeated three times to give a white solid and was used directly for the next step. MS m/z: 436.2 (M+H⁺).

### Step 1f: 1-(4-((2-(1H-indazol-4-yl)-4-morpholinothieno[3,2-d]pyrimidin-6-yl)methyl)piperazin-1-yl)prop-2-en-1-one (II-a-2)

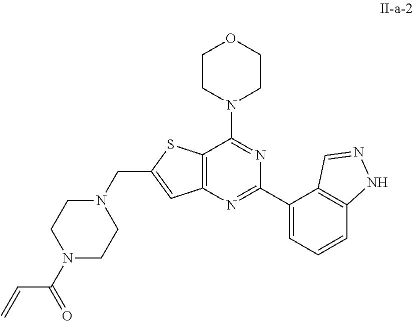

To a solution of Intermediate 1e (10 mg, 0.02 mmol) and acrylic acid (2.0 mg, 0.025 mmol) in 1.0 ml of anhydrous acetonitrile was added HATU (9.1 mg, 0.024 mmol) and DIEA (15 mg, 0.1 mmol) at −40° C. while stirring. The reaction mixture was stirred for 10 min at ˜−10° C. A 10-ml portion of EtOAc and 5 ml of NaHCO₃ aqueous solution were added. The organic layer was separated and was dried over Na₂SO₄. After removal of solvent, the crude product was subject to chromatography on silica gel (eluents: EtOAc/hexane 9:1). A total of 6 mg of the title compound was obtained. MS m/z: 490.2 (M+H⁺). ¹H NMR (400 MHz, CDCl₃): δ: 9.01 (1H d, J=0.88 Hz), 8.27 (1H d, J=7.32 Hz), 7.58 (1H d, J=7.0 Hz), 7.51 (1H t, J=6.84 Hz), 7.39 (1H, s), 6.56 (1H dd, J=10.56, 16.96 Hz), 6.32 (1H d, 16.96 Hz), 5.70 (1H d, 10.52 Hz), 4.09 (4H, m), 3.93 (6H, m), 3.79 (2H, s), 3.62 (2H, s), 2.60 (4H, s).

In similar fashion, using Intermediate 1e and coupling with acryloyl chloride (2.5 eqiv.), 1-(4-((2-(1-acryloyl-1H-indazol-4-yl)-4-morpholinothieno[3,2-d]pyrimidin-6-yl)methyl)piperazin-1-yl)prop-2-en-1-one (II-a-14) was prepared:

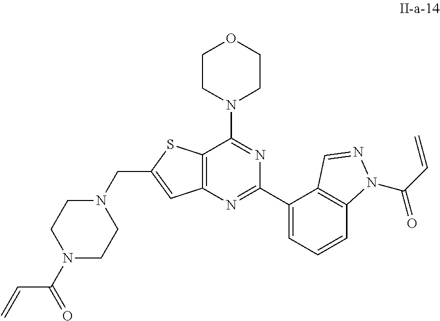

MS m/z: 544.1 (M+H⁺).

In similar fashion, using Intermediate 1e and coupling with CDI, (4-((2-(1H-indazol-4-yl)-4-morpholinothieno[3,2-d]pyrimidin-6-yl)methyl)piperazin-1-yl)(1H-imidazol-1-yl)methanone (II-a-15) was prepared:

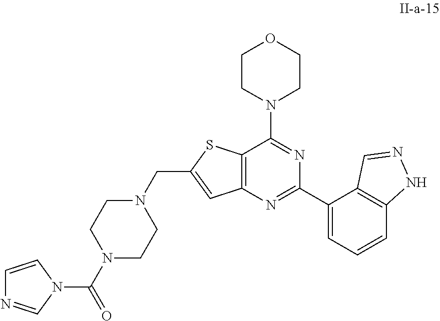

MS m/z: 530.2 (M+H⁺).

In similar fashion, using (Intermediate 1e and coupling with 2-chloroethanesulfonyl chloride in the presence of TEA, 4-(2-(1H-indazol-4-yl)-6-(4-(vinylsulfonyl)piperazin-1-yl)methyl)thieno[3,2-d]pyrimidin-4-yl)morpholine (II-a-1) was prepared:

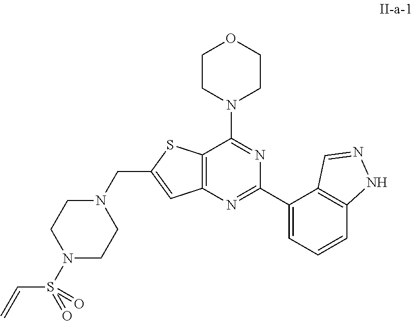

MS m/z: 526.2 (M+H⁺).

In similar fashion, the following compound was prepared by coupling Intermediate 1e and an appropriate acid:

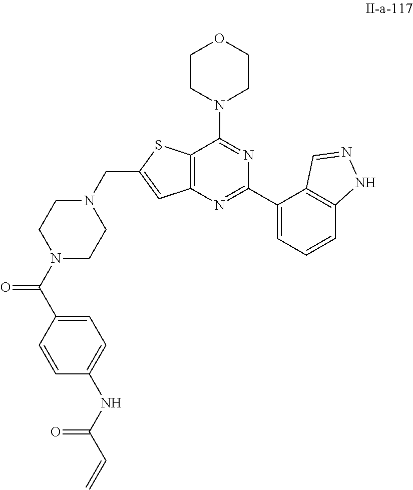

N-(4-(4-((2-(1H-indazol-4-yl)-4-morpholinothieno[3,2-d]pyrimidin-6-yl)methyl)piperazine-1-carbonyl)phenyl)acrylamide (II-a-117): MS: m/z 609.2 (ES+).

In similar fashion, the following compound was prepared by coupling Intermediate 1e and an appropriate sulfonyl chloride:

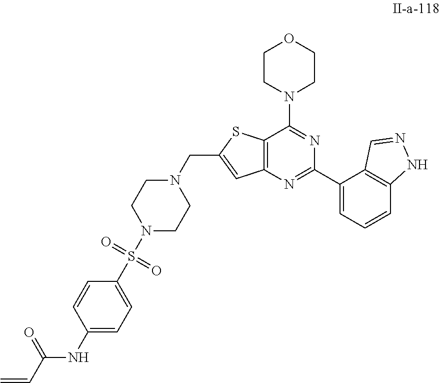

N-(4-(4-((2-(1H-indazol-4-yl)-4-morpholinothieno[3,2-d]pyrimidin-6-yl)methyl)piperazin-1-ylsulfonyl)phenyl)acrylamide (II-a-118): MS: m/z 645.2 (ES+).

### Example 2

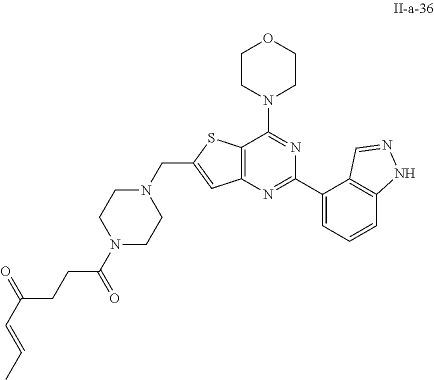

(E)-1-(4-((2-(1H-indazol-4-yl)-4-morpholinothieno[3,2-d]pyrimidin-6-yl)methyl)piperazin-1-yl)hept-5-ene-1,4-dione (II-a-36): The title compound was prepared according to the steps and intermediates as described below.

### Step 2a: (E)-4-oxohept-5-enoic acid (Intermediate 2a)

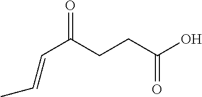

To a solution of succinic anhydride (0.50 g 5.0 mmol) in 20.0 ml of anhydrous THF was added 1-propenyl magnesium bromide (0.5 M in THF, 18.0 mL, 9.0 mmol) at −78° C. slowly. The reaction mixture was stirred for 1 h at −78° C. 1 N HCl (9.0 ml) aqueous solution was added and the mixture was slowly warmed up to RT. The pH was adjusted to ˜3 by 1 N HCl. The THF was then removed under vacuum and the remaining aqueous was extracted by DCM (3×20 mL). The organic layer was dried over Na₂SO₄, filtered and the solvent was removed. The residue was purified by chromatography on silica gel (eluents: EtOAc/hexane 1:1) to provide the acid. ¹H NMR (400 MHz, CDCl₃): δ: 6.90 (1H dq, J=6.88 Hz, 16.0 Hz), 6.15 (1H dq, J=16.0 Hz, 1.68 Hz), 2.87 (2H t, J=6.64 Hz), 2.67 (2H t, J=6.64 Hz), 1.91 (3H dd, J=1.44 Hz, 6.84 Hz).

### Step 2b: (E)-1-(4-((2-(1H-indazol-4-yl)-4-morpholinothieno[3,2-d]pyrimidin-6-yl)methyl)piperazin-1-yl)hept-5-ene-1,4-dione (II-a-36)

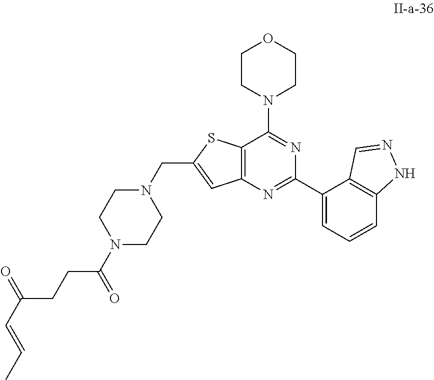

The title compound was prepared by coupling (E)-4-oxohept-5-enoic acid obtained above with Intermediate 1e using HATU following the procedure described in Step 1f. MS m/z: 560.2 (M+H⁺). ¹H NMR (400 MHz, DMSO-d6): δ: 8.886 (1H bt), 8.228 (1H dd), 7.667 (1H dt), 7.514 (1H t), 7.47 (1H, m), 6.86 (1H dq), 6.13 (1H dq), 4.01 (4H, bt), 3.92 (2H, s), 3.84 (4H, bt), 3.49 (4H, dt), 2.77 (2H, bt), 2.55 (2H, bt), 1.865 (3H, dd).

In similar fashion, the following compounds were prepared by coupling Intermediate 1e and a proper acid produced following step 2a:

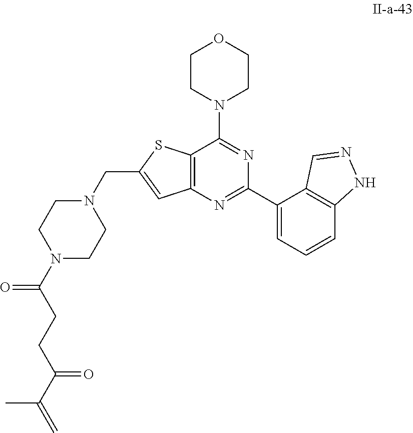

1-(4-((2-(1H-indazol-4-yl)-4-morpholinothieno[3,2-d]pyrimidin-6-yl)methyl)piperazin-1-yl)-5-methylhex-5-ene-1,4-dione (II-a-43): MS m/z: 560.3 (M+H⁺); ¹H NMR (400 MHz, DMSO-d6): δ: 8.885 (1H t), 8.23 (1H dd), 7.67 (1H dt), 7.515 (1H s), 7.472 (1H, q), 6.096 (1H bt), 5.846 (1H bt), 4.01 (4H, t), 3.93 (1H, s), 3.84 (4H, t), 3.5 (4H, dt), 2.93 (2H, t), 2.52 (6H, m).

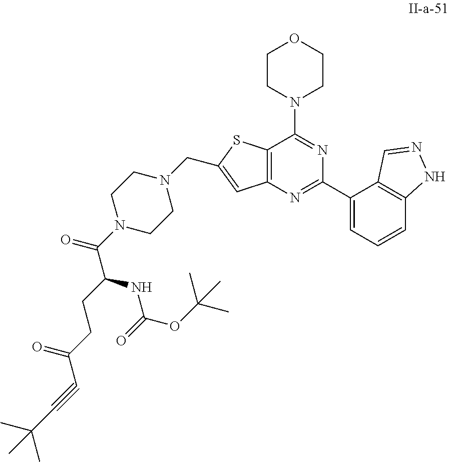

(S)-tert-butyl 1-(4-((2-(1H-indazol-4-yl)-4-morpholinothieno[3,2-d]pyrimidin-6-yl)methyl)piperazin-1-yl)-8,8-dimethyl-1,5-dioxonon-6-yn-2-ylcarbamate (II-a-51): MS m/z: 729.3 (M+H⁺).

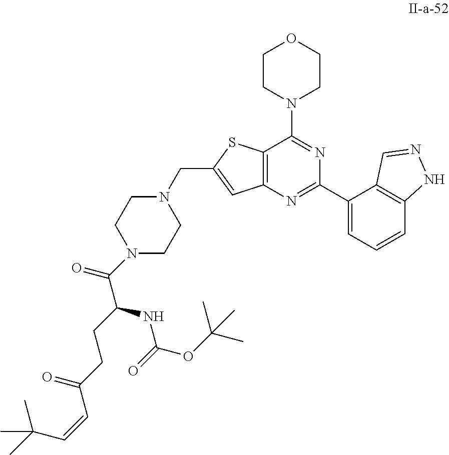

(S)-tert-butyl 1-(4-((2-(1H-indazol-4-yl)-4-morpholinothieno[3,2-d]pyrimidin-6-yl)methyl)piperazin-1-yl)-8,8-dimethyl-1,5-dioxonon-6-en-2-ylcarbamate (II-a-52): MS m/z: 731.3 (M+H⁺).

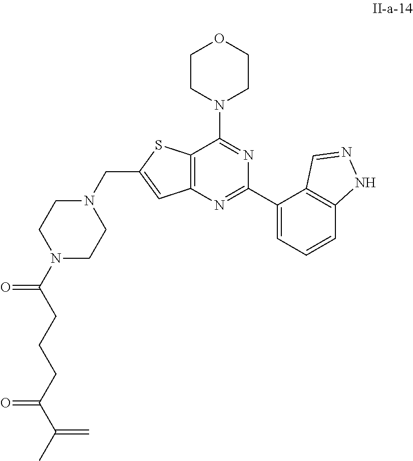

1-(4-((2-(1H-indazol-4-yl)-4-morpholinothieno[3,2-d]pyrimidin-6-yl)methyl)piperazin-1-yl)-6-methylhept-6-ene-1,5-dione (II-a-14): MS m/z: 574.2 (M+H⁺); ¹H NMR (400 MHz, DMSO-d6): δ: 8.89 (1H bt), 8.23 (1H d), 7.67 (1H dt), 7.51 (1H, s), 7.47 (1H q), 6.06 (1H bt), 5.85 (1H, m), 4.01 (4H, bt), 3.92 (2H, s), 3.84 (4H, bt), 3.48 (4H, bs), 2.75 (2H, t), 2.31 (2H, t), 1.78 (3H, s), 1.71 (2H, m).

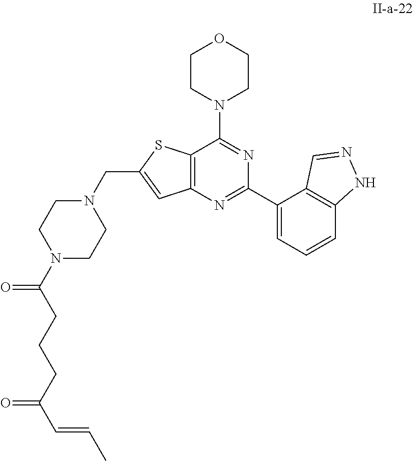

(E)-1-(4-((2-(1H-indazol-4-yl)-4-morpholinothieno[3,2-d]pyrimidin-6-yl)methyl)piperazin-1-yl)oct-6-ene-1,5-dione (II-a-22): MS m/z: 574.2 (M+H⁺); ¹H NMR (400 MHz, DMSO-d6): δ: 8.88 (1H m), 8.225 (1H dd), 7.67 (1H dt), 7.51 (1H, s), 7.47 (1H q), 6.85 (1H dq), 6.09 (1H, dq), 4.01 (4H, bt), 3.92 (2H, s), 3.84 (4H, bt), 3.48 (4H, bm), 2.58 (2H, t), 2.3 (2H, t), 1.85 (3H, dd), 1.69 (2H, m).

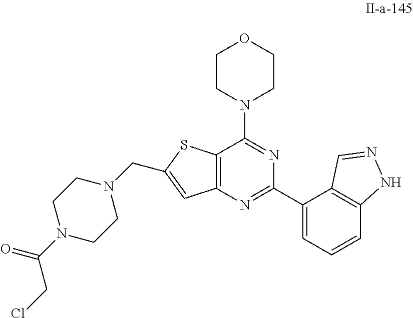

1-(4-((2-(1H-indazol-4-yl)-4-morpholinothieno[3,2-d]pyrimidin-6-yl)methyl)piperazin-1-yl)-2-chloroethanone (II-a-145): MS: m/z 514.3 (ES+)

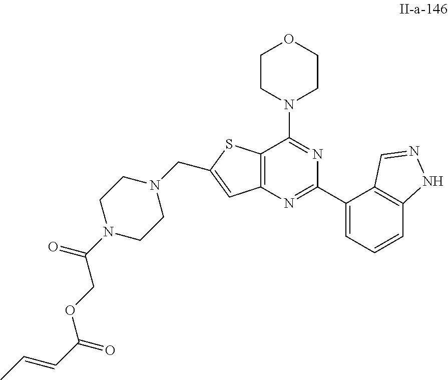

(E)-2-(4-((2-(1H-indazol-4-yl)-4-morpholinothieno[3,2-d]pyrimidin-6-yl)methyl)piperazin-1-yl)-2-oxoethyl but-2-enoate (II-a-146): MS: m/z 562.3 (ES+).

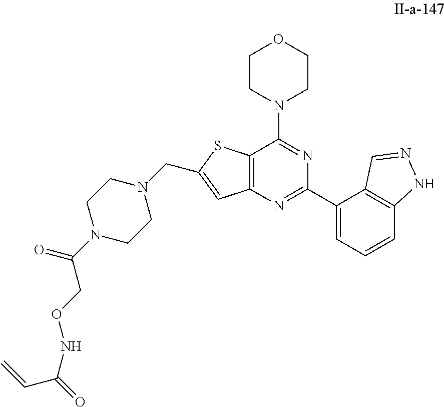

N-(2-(4-((2-(1H-indazol-4-yl)-4-morpholinothieno[3,2-d]pyrimidin-6-yl)methyl)piperazin-1-yl)-2-oxoethoxy)acrylamide (II-a-147): MS: m/z 563.3 (ES+)

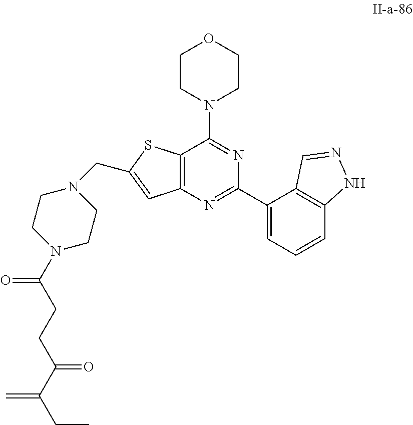

1-(4-((2-(1H-indazol-4-yl)-4-morpholinothieno[3,2-d]pyrimidin-6-yl)methyl)piperazin-1-yl)-5-methyleneheptane-1,4-dione (II-a-86). MS: m/z 574.9 (ES+).

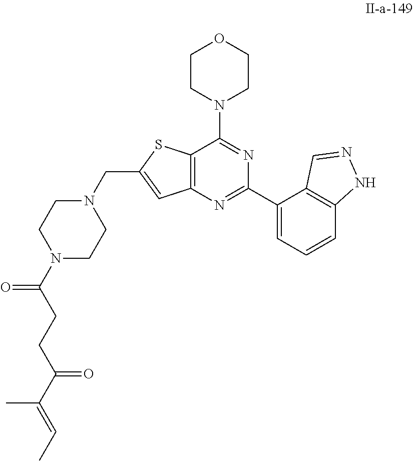

(E)-1-(4-((2-(1H-indazol-4-yl)-4-morpholinothieno[3,2-d]pyrimidin-6-yl)methyl)piperazin-1-yl)-5-methylhept-5-ene-1,4-dione (II-a-149). MS: m/z 574.8 (ES+).

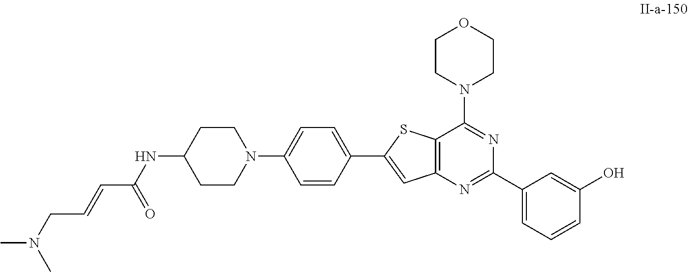

(E)-4-(dimethylamino)-N-(1-(4-(2-(3-hydroxyphenyl)-4-morpholinothieno[3,2-d]pyrimidin-6-yl)phenyl)piperidin-4-yl)but-2-enamide (II-a-150). MS: m/z 599.3 (ES+).

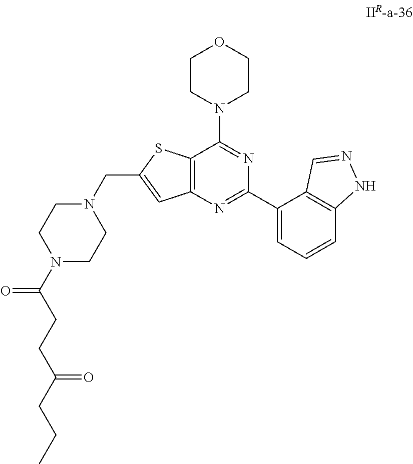

1-(4-((2-(1H-indazol-4-yl)-4-morpholinothieno[3,2-d]pyrimidin-6-yl)methyl)piperazin-1-yl)heptane-1,4-dione (II^{R}-a-36): The title compound was prepared via hydrogenation of II-a-36 using 5% Pd/C in MeOH under hydrogen. MS: m/z 562.3 (ES+).

In a similar fashion as shown in Examples 1 and 2, using 2-aminopyrimidine-5-boronic acid to couple with Intermediate 1c, the following compound was prepared:

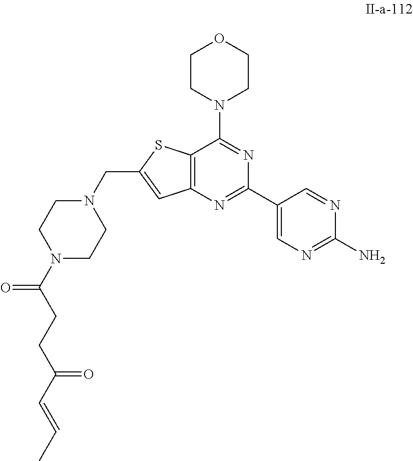

(E)-1-(4-((2-(2-aminopyrimidin-5-yl)-4-morpholinothieno[3,2-d]pyrimidin-6-yl)methyl)piperazin-1-yl)hept-5-ene-1,4-dione (II-a-112): MS: m/z 537.3 (ES+).

In a similar fashion as shown in Examples 1 and 2, using 1H-pyrrolo[2,3-b]pyridin-4-ylboronic acid to couple with Intermediate 1c, the following compounds were prepared:

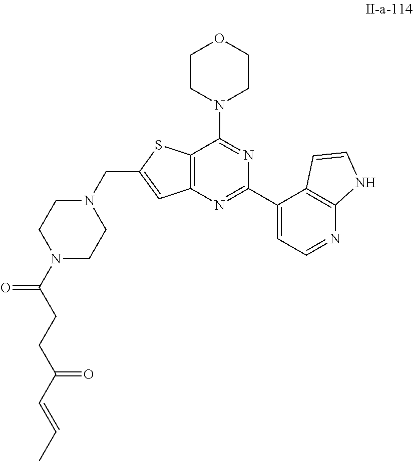

(E)-1-(4-((4-morpholino-2-(1H-pyrrolo[2,3-b]pyridin-4-yl)thieno[3,2-d]pyrimidin-6-yl)methyl)piperazin-1-yl)hept-5-ene-1,4-dione (II-a-114): MS: m/z 560.3 (ES+).

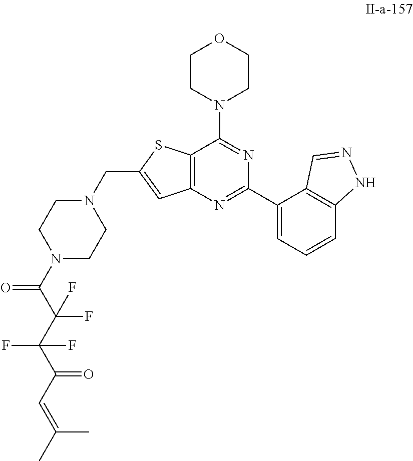

1-(4-((2-(1H-indazol-4-yl)-4-morpholinothieno[3,2-d]pyrimidin-6-yl)methyl)piperazin-1-yl)-2,2,3,3-tetrafluoro-6-methylhept-5-ene-1,4-dione (II-a-157). MS: m/z 646.1 (ES+).

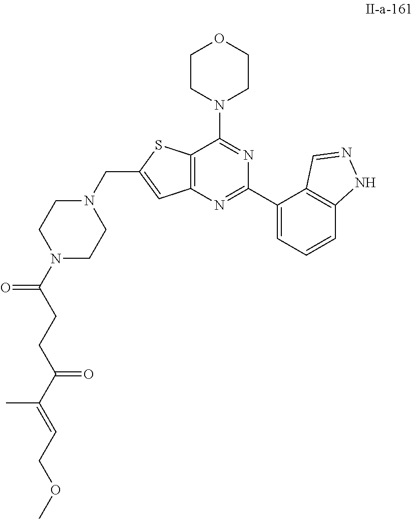

(E)-1-(4-((2-(1H-indazol-4-yl)-4-morpholinothieno[3,2-d]pyrimidin-6-yl)methyl)piperazin-1-yl)-7-methoxy-5-methylhept-5-ene-1,4-dione (II-a-161). MS: m/z 604.8 (ES+).

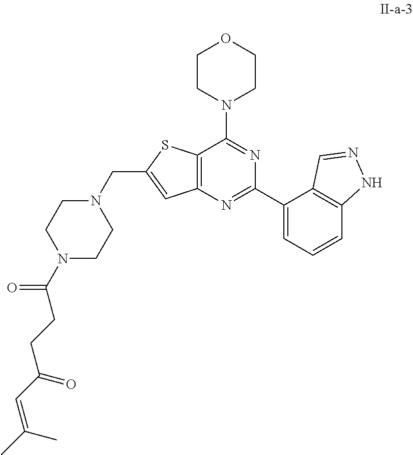

1-(4-((2-(1H-indazol-4-yl)-4-morpholinothieno[3,2-d]pyrimidin-6-yl)methyl)piperazin-1-yl)-6-methylhept-5-ene-1,4-dione (II-a-3). MS: m/z 574.2 (ES+)

### Example 3

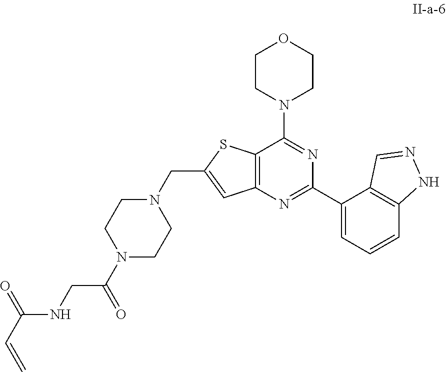

N-(2-(4-((2-(1H-indazol-4-yl)-4-morpholinothieno[3,2-d]pyrimidin-6-yl)methyl)piperazin-1-yl)-2-oxoethyl)acrylamide (II-a-6): The title compound was prepared according to the steps and intermediates as described below.

### Step 3a: tert-butyl 2-(4-((2-(1H-indazol-4-yl)-4-morpholinothieno[3,2-d]pyrimidin-6-yl)methyl)piperazin-1-yl)-2-oxoethylcarbamate (Intermediate 3a)

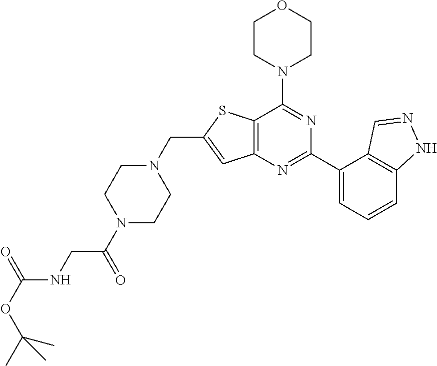

The title compound was prepared by coupling BOC-Gly-OH with Intermediate 1e using HATU following the procedure described in Step 1f. MS m/z: 593.2 (M+H⁺).

### Step 3b: 1-(4-((2-(1H-indazol-4-yl)-4-morpholinothieno[3,2-d]pyrimidin-6-yl)methyl)piperazin-1-yl)-2-aminoethanone hydrochloride (Intermediate 3b)

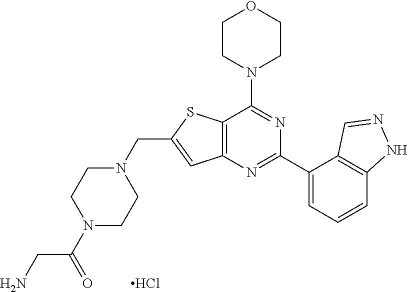

The title compound was made by the de-BOC procedure described in Step 1e. MS m/z: 493.2 (M+H⁺).

### Step 3c: N-(2-(4-((2-(1H-indazol-4-yl)-4-morpholinothieno[3,2-d]pyrimidin-6-yl)methyl)piperazin-1-yl)-2-oxoethyl)acrylamide (II-a-6)

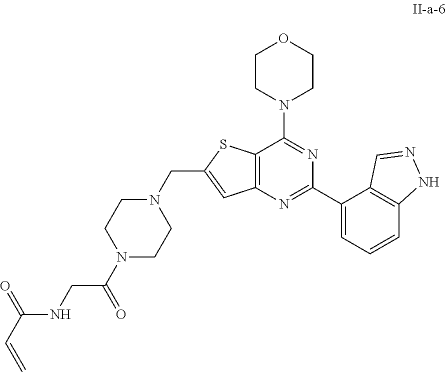

The title compound was prepared by coupling acrylic acid with Intermediate 3b using HATU following the procedure described in Step 1f. MS m/z: 547.3 (M+H⁺). ¹H NMR (400 MHz, CDCl₃): δ: 9.01 (1H d, J=0.92 Hz), 8.28 (1H d, J=7.32 Hz), 7.59 (1H d, J=7.32 Hz), 7.51 (1H t, J=7.32 Hz), 7.40 (1H, s), 6.75 (1H, s), 6.25 (2H m), 5.70 (1H d, 10.52 Hz), 4.11 (6H, m), 3.91 (6H, m), 3.72 (2H, t), 3.51 (2H, t), 2.60 (4H, s).

In similar fashion, using Intermediate 3b and coupling with 4-oxo-hept-5-enoic acid (from step 2a), (E)-N-(2-(4-((2-(1H-indazol-4-yl)-4-morpholinothieno[3,2-d]pyrimidin-6-yl)methyl)piperazin-1-yl)-2-oxoethyl)-4-oxohept-5-enamide (II-a-16) was prepared:

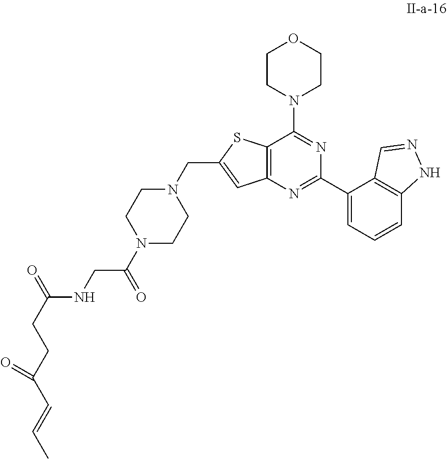

MS m/z: 617.2 (M+H⁺).

In similar fashion, the following compounds were prepared by coupling Intermediate 3b and a proper acid produced following step 2a:

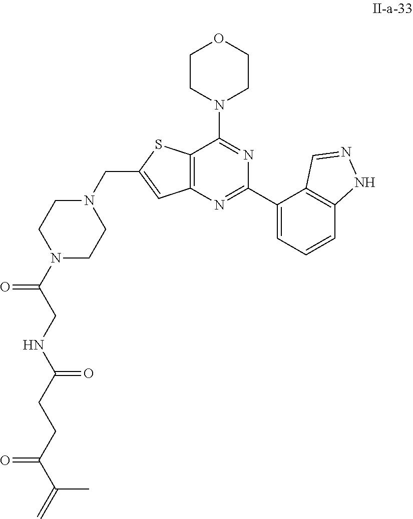

N-(2-(4-((2-(1H-indazol-4-yl)-4-morpholinothieno[3,2-d]pyrimidin-6-yl)methyl)piperazin-1-yl)-2-oxoethyl)-5-methyl-4-oxohex-5-enamide (II-a-33): MS m/z: 617.2 (M+H⁺).

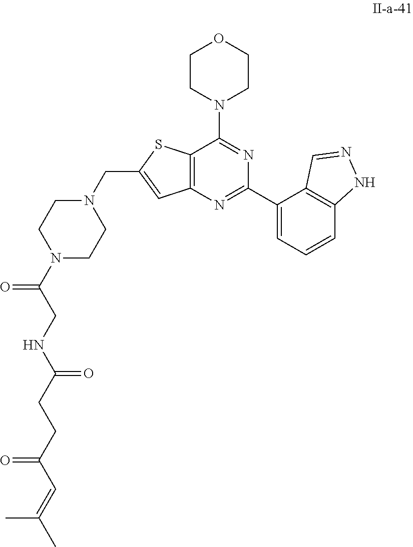

N-(2-(4-((2-(1H-indazol-4-yl)-4-morpholinothieno[3,2-d]pyrimidin-6-yl)methyl)piperazin-1-yl)-2-oxoethyl)-6-methyl-4-oxohept-5-enamide (II-a-41): MS m/z: 631.2 (M+H⁺).

The following compounds were prepared by starting with Intermediate 1e and following the procedures or procedure combinations described in previous examples:

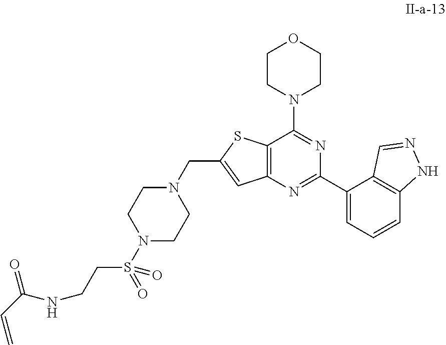

N-(2-(4-((2-(1H-indazol-4-yl)-4-morpholinothieno[3,2-d]pyrimidin-6-yl)methyl)piperazin-1-ylsulfonyl)ethyl)acrylamide (II-a-13): MS m/z: 597.2 (M+H⁺).

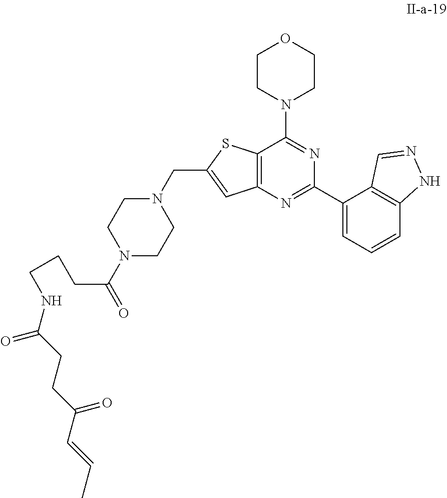

(E)-N-(4-(4-((2-(1H-indazol-4-yl)-4-morpholinothieno[3,2-d]pyrimidin-6-yl)methyl)piperazin-1-yl)-4-oxobutyl)-4-oxohept-5-enamide (II-a-19): MS m/z: 645.3 (M+H⁺).

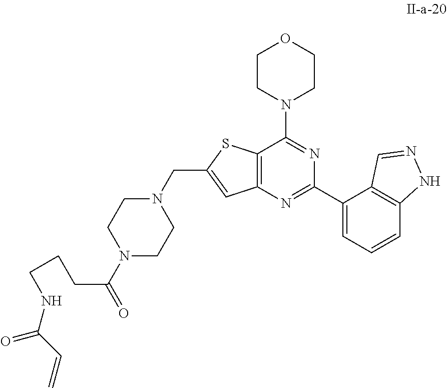

N-(4-(4-((2-(1H-indazol-4-yl)-4-morpholinothieno[3,2-d]pyrimidin-6-yl)methyl)piperazin-1-yl)-4-oxobutyl)acrylamide (II-a-20): MS m/z: 575.2 (M+H⁺).

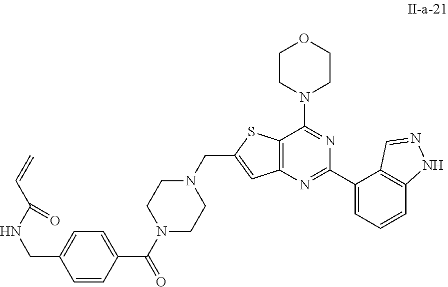

N-(4-(4-((2-(1H-indazol-4-yl)-4-morpholinothieno[3,2-d]pyrimidin-6-yl)methyl)piperazine-1-carbonyl)benzyl)acrylamide (II-a-21): MS m/z: 623.2 (M+H⁺).

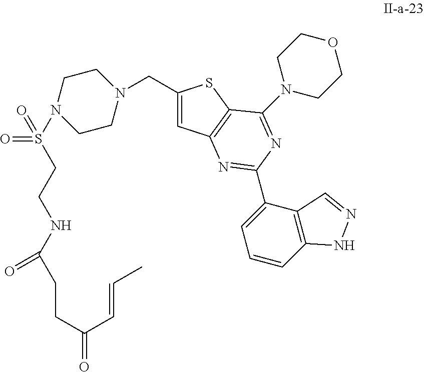

(E)-N-(2-(4-((2-(1H-indazol-4-yl)-4-morpholinothieno[3,2-d]pyrimidin-6-yl)methyl)piperazin-1-ylsulfonyl)ethyl)-4-oxohept-5-enamide (II-a-23): MS m/z: 667.1 (M+H⁺).

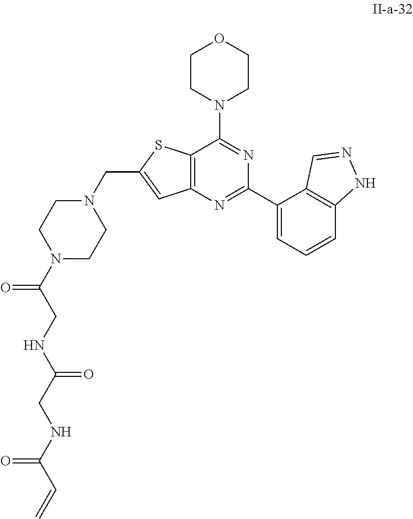

N-(2-(2-(4-((2-(1H-indazol-4-yl)-4-morpholinothieno[3,2-d]pyrimidin-6-yl)methyl)piperazin-1-yl)-2-oxoethylamino)-2-oxoethyl)acrylamide (II-a-32): MS m/z: 604.3 (M+H⁺); ¹H NMR (400 MHz, DMSO-d6): δ: 8.89 (1H s), 8.42 (1H t), 8.23 (1H d), 7.97 (1H t), 7.67 (1H, d), 7.52 (1H s), 7.47 (1H t), 6.32 (1H, q), 6.2 (1H, dd), 5.62 (1H, dd), 3.92 (14H, m), 3.48 (4H, m).

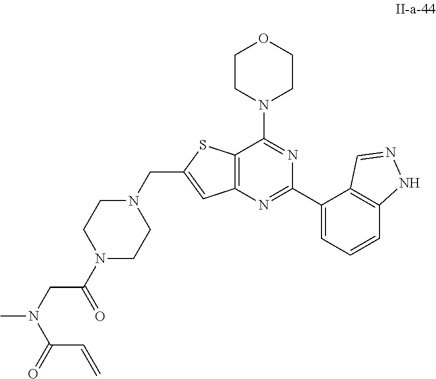

N-(2-(4-((2-(1H-indazol-4-yl)-4-morpholinothieno[3,2-d]pyrimidin-6-yl)methyl)piperazin-1-yl)-2-oxoethyl)-N-methylacrylamide (II-a-44): MS m/z: 561.2 (M+H⁺).

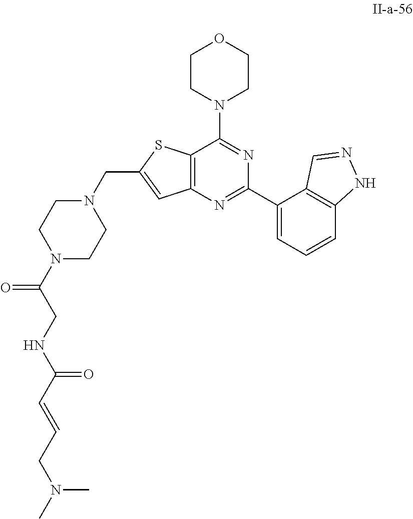

(E)-N-(2-(4-((2-(1H-indazol-4-yl)-4-morpholinothieno[3,2-d]pyrimidin-6-yl)methyl)piperazin-1-yl)-2-oxoethyl)-4-(dimethylamino)but-2-enamide (II-a-56): MS m/z: 604.2 (M+H⁺); ¹H NMR (400 MHz, DMSO-d6): δ: 8.89 (1H s), 8.23 (1H d), 8.14 (1H t), 7.67 (1H d), 7.515 (1H, s), 7.47 (1H t), 6.56 (1H dt), 6.17 (1H, dt), 4.02 (6H, m), 3.93 (2H, s), 3.84 (4H, bt), 3.49 (4H, bs), 2.98 (2H, bd), 2.14 (6H, s).

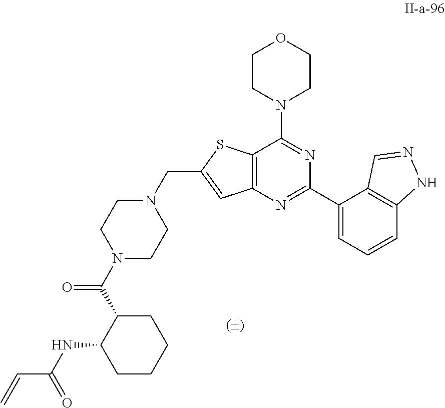

(±)-cis-N-(2-(4-((2-(1H-indazol-4-yl)-4-morpholinothieno[3,2-d]pyrimidin-6-yl)methyl)piperazine-1-carbonyl)cyclohexyl)acrylamide: MS m/z: 615.2 (M+H⁺).

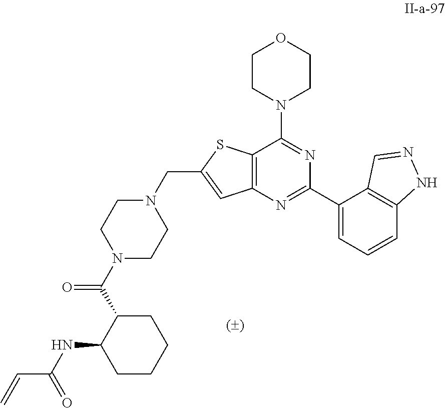

(±)-trans-N-(2-(4-((2-(1H-indazol-4-yl)-4-morpholinothieno[3,2-d]pyrimidin-6-yl)methyl)piperazine-1-carbonyl)cyclohexyl)acrylamide: MS m/z: 615.3 (M+H⁺).

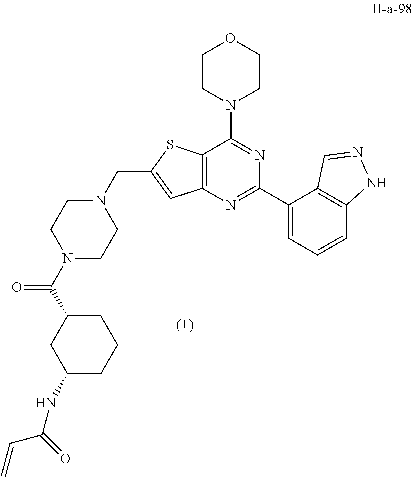

(±)-cis-N-(3-(4-((2-(1H-indazol-4-yl)-4-morpholinothieno[3,2-d]pyrimidin-6-yl)methyl)piperazine-1-carbonyl)cyclohexyl)acrylamide: MS m/z: 615.3 (M+H⁺).

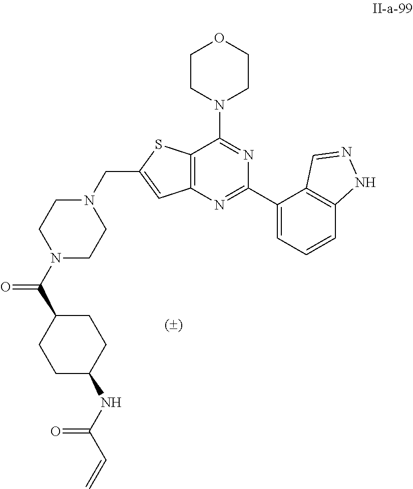

(±)-cis-N-(4-(4-((2-(1H-indazol-4-yl)-4-morpholinothieno[3,2-d]pyrimidin-6-yl)methyl)piperazine-1-carbonyl)cyclohexyl)acrylamide: MS m/z: 615.3 (M+H⁺).

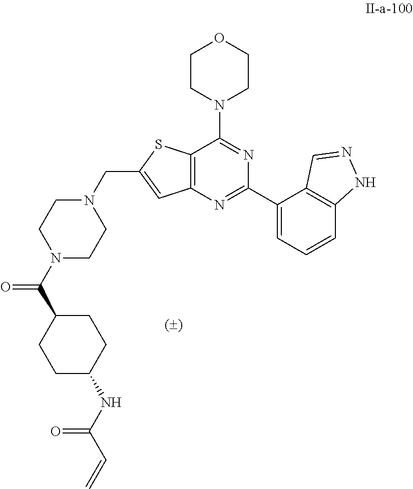

(±)-trans-N-(4-(4-((2-(1H-indazol-4-yl)-4-morpholinothieno[3,2-d]pyrimidin-6-yl)methyl)piperazine-1-carbonyl)cyclohexyl)acrylamide: MS m/z: 615.3 (M+H⁺); ¹H NMR (400 MHz, DMSO-d6): δ: 8.88 (1H s), 8.23 (1H d), 7.98 (1H d), 7.67 (1H, d), 7.5 (1H s), 7.47 (1H, t), 6.2 (1H, q), 6.06 (1H, dd), 5.55 (1H, dd), 4.01 (4H, bt), 3.92 (2H, s), 3.84 (4H, bt), 3.52 (5H, dm), 2.09 (1H, s), 1.76 (4H, bdd), 1.42 (2H, bq), 1.24 (2H, bq).

### Example 4

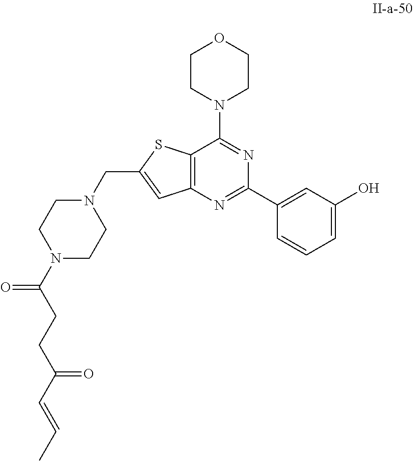

(E)-1-(4-((2-(3-hydroxyphenyl)-4-morpholinothieno[3,2-d]pyrimidin-6-yl)methyl)piperazin-1-yl)hept-5-ene-1,4-dione (II-a-50): The title compound was prepared according to the steps and intermediates as described below.

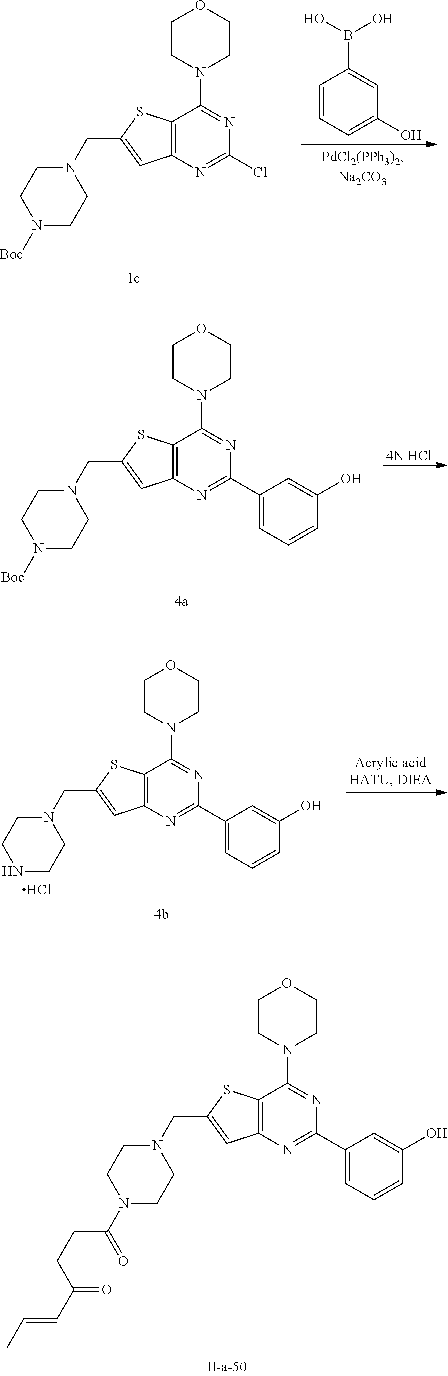

### Step 4a: tert-butyl 4-((2-(3-hydroxyphenyl)-4-morpholinothieno[3,2-d]pyrimidin-6-yl)methyl)piperazine-1-carboxylate (Intermediate 4a)

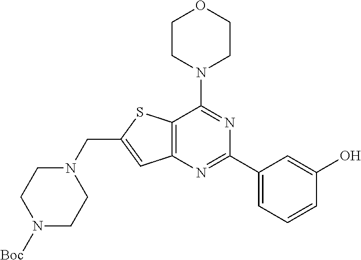

Intermediate 1c (305 mg, 0.67 mmol), 3-hydroxyphenylboronic acid (139 mg, 1.0 mmol), tetrakis(triphenylphosphine)palladium (51 mg, 0.067 mmol) and sodium carbonate (214 mg, 2 mmol.) were dissolved in toluene/ethanol/water (6 mL/3.6 mL/1.8 mL). The solution was degassed and flushed with N₂. The reaction mixture was heated to 120° C. for 1 hr in a sealed vial. The solvent was removed under vacuum and the residue was purified by chromatography on silica gel (eluents: EtOAc/hexane 5:5). A total of 360 mg as a yellow foam of the title compound was obtained. MS m/z: 512.3 (M+1).

### Step 4b: 3-(4-morpholino-6-(piperazin-1-ylmethyl)thieno[3,2-d]pyrimidin-2-yl)phenol hydrochloride (Intermediate 4b)

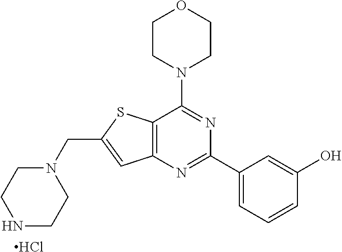

Intermediate 4a (360 mg, 0.7 mmol) was dissolved in 500 uL of 4N HCl and DCM (5 mL); reaction was stirred for 3 hours at room temperature. After removal of solvents, gave a white solid (350 mg) and was used directly for the next step. MS m/z: 412.1 (M+H⁺).

### Step 4c: (E)-1-(4-((2-(3-hydroxyphenyl)-4-morpholinothieno[3,2-d]pyrimidin-6-yl)methyl)piperazin-1-yl)hept-5-ene-1,4-dione (II-a-50)

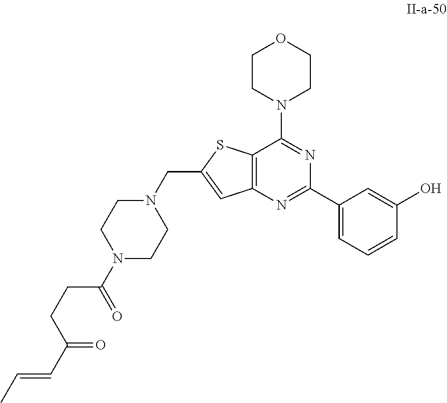

The title compound was prepared by coupling (E)-4-oxohept-5-enoic acid from step 2a with Intermediate 4b using HATU following the procedure described in Step 1f. MS m/z: 536.3 (M+H⁺). ¹H NMR (400 MHz, DMSO-d6): δ: 9.45 (1H s,), 7.85 (2H m,), 7.39 (1H s,), 7.26 (1H t,), 6.86 (2H, m), 6.13 (1H dd,), 3.97 (4H, bt), 3.89 (2H, s), 3.85 (4H, bt), 3.48 (4H, bt), 2.76 (2H, t), 2.54 (2H, t), 1.86 (3H, dd).

In similar fashion, 1-(4-((2-(3-hydroxyphenyl)-4-morpholinothieno[3,2-d]pyrimidin-6-yl)methyl)piperazin-1-yl)-5-methylhex-5-ene-1,4-dione (II-a-49) was prepared by coupling Intermediate 4b and 5-methyl-4-oxohex-5-enoic acid produced following step 2a.

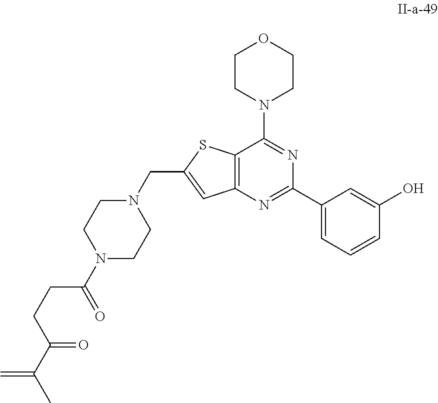

MS m/z: 536.2 (M+H⁺); ¹H NMR (400 MHz, DMSO-d6): δ: 9.5 (1H s), 7.84 (2H m), 7.39 (1H s), 7.26 (1H t), 6.85 (1H, m), 6.09 (1H s), 5.845 (1H bs), 3.97 (4H, bt), 3.9 (1H, s), 3.88 (4H, bt), 3.49 (4H, dt), 2.925 (2H, t), 2.5 (6H, m).

The following compounds were prepared by starting with Intermediate 4b and following the procedures or procedure combinations described in previous examples:

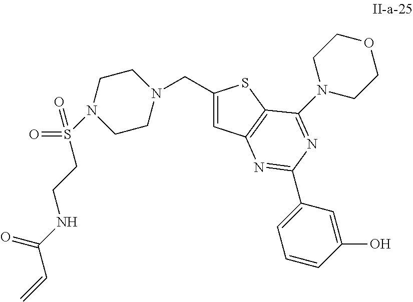

N-(2-(4-((2-(3-hydroxyphenyl)-4-morpholinothieno[3,2-d]pyrimidin-6-yl)methyl)piperazin-1-ylsulfonyl)ethyl)acrylamide (II-a-25): MS m/z: 573.2 (M+H⁺).

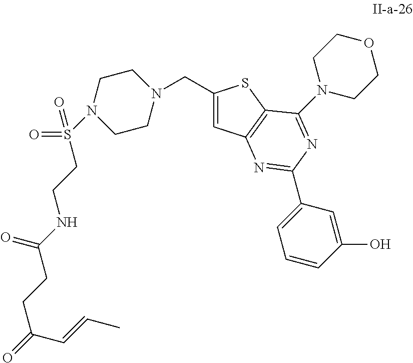

(E)-N-(2-(4-((2-(3-hydroxyphenyl)-4-morpholinothieno[3,2-d]pyrimidin-6-yl)methyl)piperazin-1-ylsulfonyl)ethyl)-4-oxohept-5-enamide (II-a-26): MS m/z: 643.2 (M+H⁺).

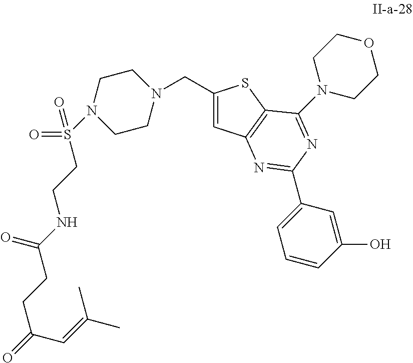

N-(2-(4-((2-(3-hydroxyphenyl)-4-morpholinothieno[3,2-d]pyrimidin-6-yl)methyl)piperazin-1-ylsulfonyl)ethyl)-6-methyl-4-oxohept-5-enamide (II-a-28): MS m/z: 657.2 (M+H⁺).

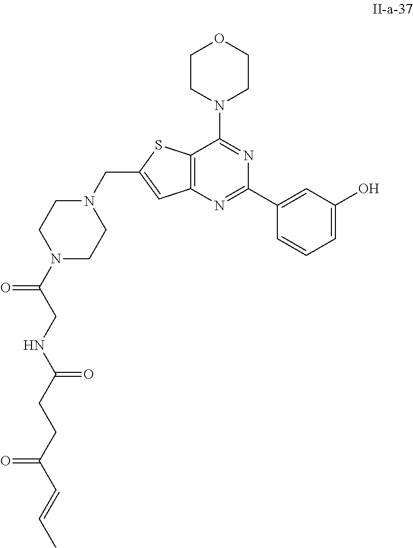

(E)-N-(2-(4-((2-(3-hydroxyphenyl)-4-morpholinothieno[3,2-d]pyrimidin-6-yl)methyl)piperazin-1-yl)-2-oxoethyl)-4-oxohept-5-enamide (II-a-37): MS m/z: 593.3 (M+H⁺).

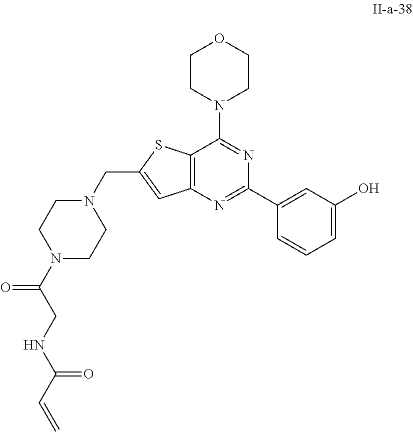

N-(2-(4-((2-(3-hydroxyphenyl)-4-morpholinothieno[3,2-d]pyrimidin-6-yl)methyl)piperazin-1-yl)-2-oxoethyl)acrylamide (II-a-38): MS m/z: 523.2 (M+H⁺).

The following compounds were prepared following the above procedures using phenylboronic acid in the place of 3-hydroxyphenylboronic acid:

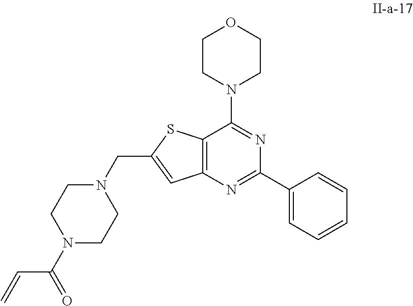

1-(4-((4-morpholino-2-phenylthieno[3,2-d]pyrimidin-6-yl)methyl)piperazin-1-yl)prop-2-en-1-one (II-a-17): MS m/z: 450.2 (M+H⁺).

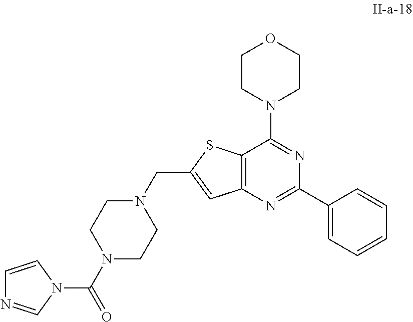

(1H-imidazol-1-yl)(4-((4-morpholino-2-phenylthieno[3,2-d]pyrimidin-6-yl)methyl)piperazin-1-yl)methanone (II-a-18): MS m/z: 490.2 (M+H⁺).

### Example 5

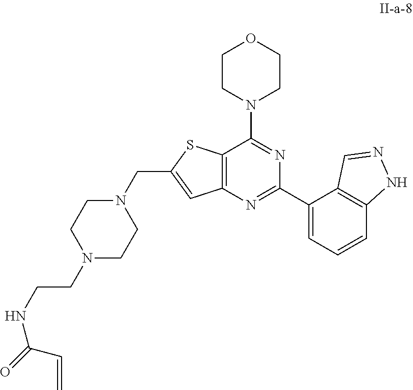

N-(2-(4-((2-(1H-indazol-4-yl)-4-morpholinothieno[3,2-d]pyrimidin-6-yl)methyl)piperazin-1-yl)ethyl)acrylamide (II-a-8): The title compound was prepared according to the scheme as described below.

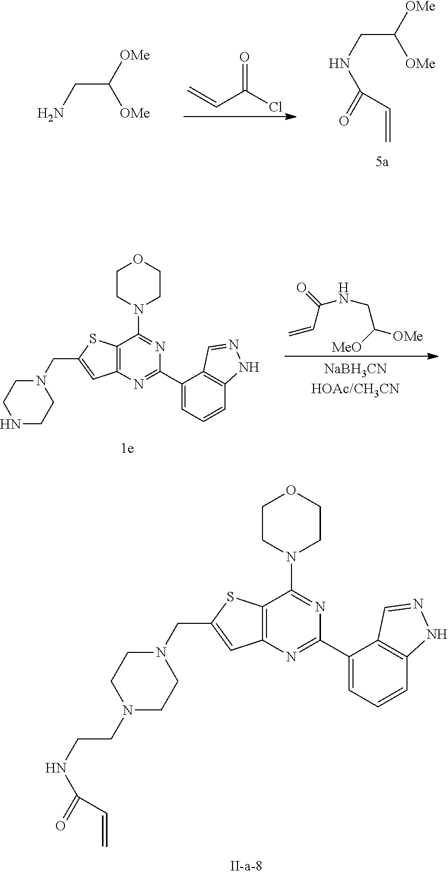

To a solution of 2,2-dimethoxyethanamine (1.0 equiv.) in dichloromethane was added acryloyl chloride (1.2 equiv.) at 0° C. slowly. Triethylamine (2.5 equiv.) was slowly introduced into the reaction mixture. The reaction was allowed to warm to RT for 1 h. The solvent was removed under vacuum and the residue was used directly in the next step.

To a solution of the product from Step 1e (20 mg, 0.04 mmol), N-(2,2-dimethoxyethyl)acrylamide obtained from above (13.5 mg, 0.08 mmol) in 0.2 ml acetic acid and 1.0 ml acetonitrile was added NaBH₃CN (5.5 mg, 0.085 mmol) at RT. The reaction was left stirring for 10 hours and was worked up by addition of ethyl acetate (10 ml) followed by aqueous NaHCO3 wash. The crude residue was purified by prep. HPLC (25% to 90% CH3CN aqueous containing 0.1% TFA) to give 8.0 mg of the title compound as a TFA salt. MS m/z: 533.2 (M+1).

### Example 6

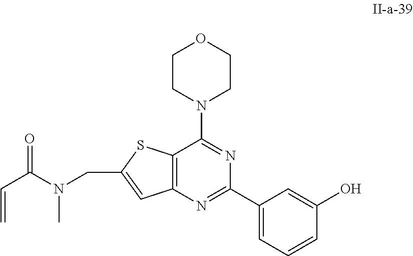

N-((2-(3-hydroxyphenyl)-4-morpholinothieno[3,2-d]pyrimidin-6-yl)methyl)-N-methylacrylamide (II-a-39): The title compound was prepared according to the steps and intermediates as described below.

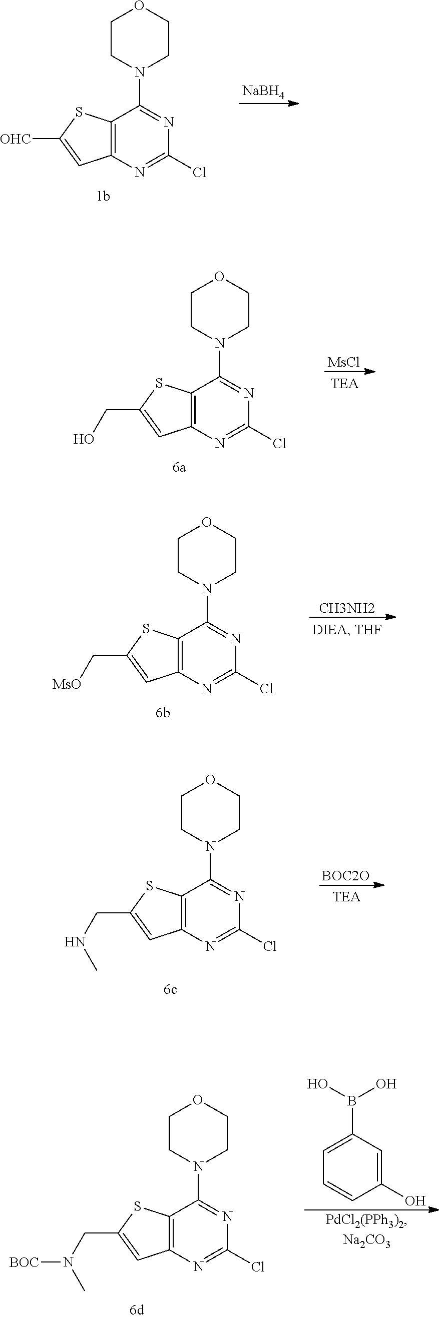

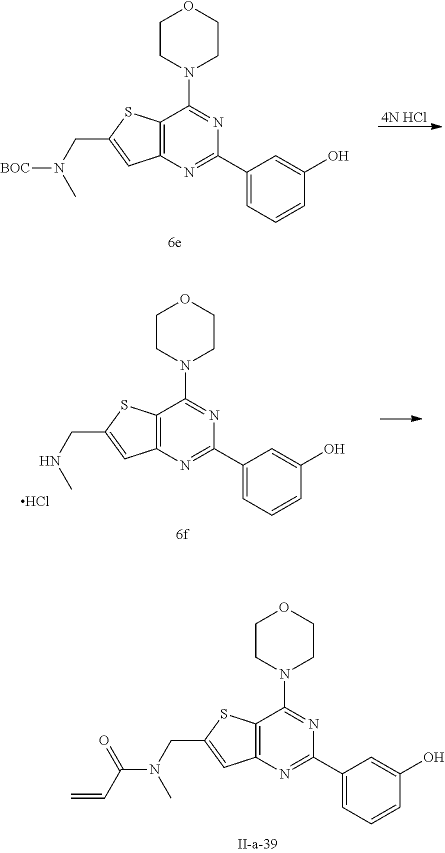

### Step 6a: (2-chloro-4-morpholinothieno[3,2-d]pyrimidin-6-yl)methanol (Intermediate 6a)

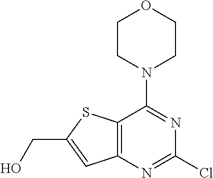

To a solution of 1b (5 g, 17.6 mmol) in MeOH (50 mL) was added NaBH₄ (0.98 g, 26.4 mmol) portion wise at 0° C. and stirred for 5 h at RT. After the completion of reaction (monitored by TLC), the volatiles were removed under reduced pressure, residue dissolved in water and extracted with DCM (3×75 mL). The combined organic phases were washed with water, dried over anhydrous Na₂SO₄ and concentrated in vacuo to afford intermediate 6a (3 g, 60%) as a light yellow solid. TLC: 80% EtOAc/Hexane (R_{f}: 0.3); ¹H-NMR (CDCl₃, 200 MHz): δ 7.21 (s, 1H), 4.98 (s, 2H), 4.0 (t, J=4.2 Hz, 4H), 3.83 (t, J=4.8 Hz, 4H); Mass: 286 [M⁺+1]

### Step 6b: (2-chloro-4-morpholinothieno[3,2-d]pyrimidin-6-yl)methyl methanesulfonate (Intermediate 6b)

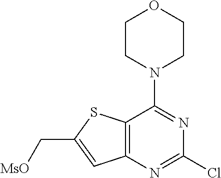

To a solution of Intermediate 6a (1 g, 3.5 mmol) in DCM (10 mL) was added TEA (1.06 g, 10.5 mmol) over a period of 10 minutes and followed by addition of mesyl chloride (0.48 g, 4.2 mmol) at 0° C. The reaction mixture was stirred for 1 h at RT. After the completion of reaction (monitored by TLC), water (25 mL) was added, extracted with DCM (2×50 mL). The combined organic phases were dried over anhydrous Na₂SO₄ and concentrated in vacuo. The crude compound was purified by silica gel column chromatography (50% EtOAc/hexane) to afford intermediate 6b (0.8 g, 62%) as a yellow solid. TLC: 80% EtOAc/Hexane (R_{f}: 0.6); ¹H-NMR (CDCl₃, 500 MHz) (SAV-A9008-009): δ 7.39 (s, 1H), 5.46 (s, 2H), 4.0 (t, J=4.5 Hz, 4H), 3.84 (t, J=5.0 Hz, 4H), 3.05 (s, 3H); Mass: 364 [M⁺+1]; Mp: 151.4° C.

### Step 6c: 1-(2-chloro-4-morpholinothieno[3,2-d]pyrimidin-6-yl)-N-methylmethanamine (Intermediate 6c)

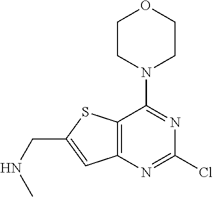

A solution of Intermediate 6b (0.24 g, 0.67 mmol), 2M methylamine in THF (2.0 ml, 4.0 mmol) and DIEA (0.35 ml, 2.0 mmol) in THF (5 mL) was stirred at RT for 2 hours. LC-MS showed the complete conversion to the product. The solvent was removed in vacuo and the crude was used directly for the next step. MS m/z: 299.1 (M+1).

### Step 6d: tert-butyl (2-chloro-4-morpholinothieno[3,2-d]pyrimidin-6-yl)methyl(methyl)carbamate (Intermediate 6d)

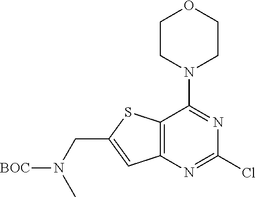

The crude Intermediate 6c, Boc₂O (0.22 g, 1.0 mmol), and TEA (0.2 ml) were dissolved in 10 ml dichloromethane and the solution was stirred for 10 hours. LC-MS showed the complete conversion to the product. The solvent was removed in vacuo and the crude was used directly for the next step. MS m/z: 399.1 (M+1).

### Step 6e: tert-butyl (2-(3-hydroxyphenyl)-4-morpholinothieno[3,2-d]pyrimidin-6-yl)methyl(methyl)carbamate (Intermediate 6e)

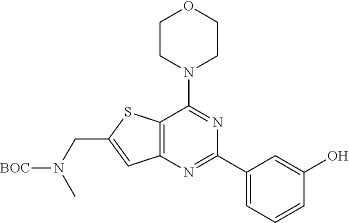

The title compound was prepared by coupling 3-hydroxyphenylboronic acid with Intermediate 6d following the procedure described in Example 4, step 4a. 0.19 g of the title compound was obtained. MS m/z: 457.1 (M+1).

### Step 6f: 3-(6-((methylamino)methyl)-4-morpholinothieno[3,2-d]pyrimidin-2-yl)phenol (Intermediate 6f)

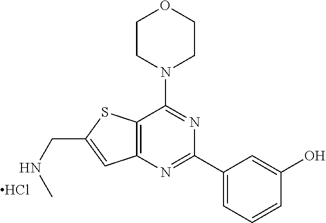

Intermediate 6e was treated with 4N HCl following the procedure described in Example 1, step 1e to afford the title compound. MS m/z: 357.1 (M+1).

### Step 6g: N-((2-(3-hydroxyphenyl)-4-morpholinothieno[3,2-d]pyrimidin-6-yl)methyl)-N-methylacrylamide (II-a-39)

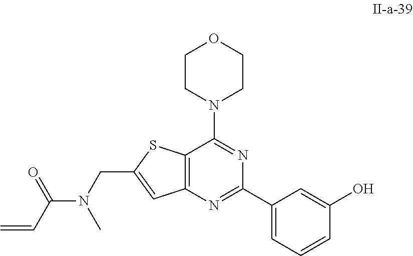

The title compound was prepared by coupling acrylic acid with Intermediate 6f using HATU following the procedure described in Step 1f. MS m/z: 411.1 (M+H⁺).

In similar fashion, using Intermediate 6f, the following compounds were prepared:

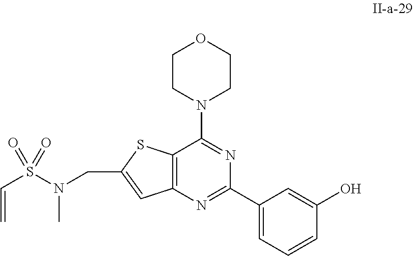

N-((2-(3-hydroxyphenyl)-4-morpholinothieno[3,2-d]pyrimidin-6-yl)methyl)-N-methylethenesulfonamide (II-a-29): MS m/z: 447.1 (M+H⁺).

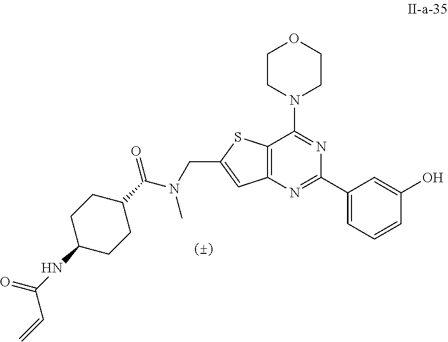

(±)-4-acrylamido-N-((2-(3-hydroxyphenyl)-4-morpholinothieno[3,2-d]pyrimidin-6-yl)methyl)-N-methylcyclohexanecarboxamide (II-a-35): MS m/z: 536.2 (M+H⁺).

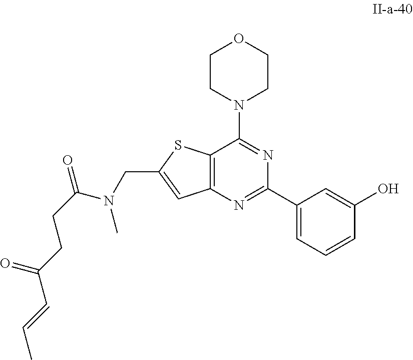

(E)-N-((2-(3-hydroxyphenyl)-4-morpholinothieno[3,2-d]pyrimidin-6-yl)methyl)-N-methyl-4-oxohept-5-enamide (II-a-40): MS m/z: 481.2 (M+H⁺).

In a similar fashion, using 2-aminopyrimidine-5-boronic acid in the Suzuki coupling step (Step 6e), and the appropriate carboxylic acid in amide formation (Step 6g), the following compounds were prepared:

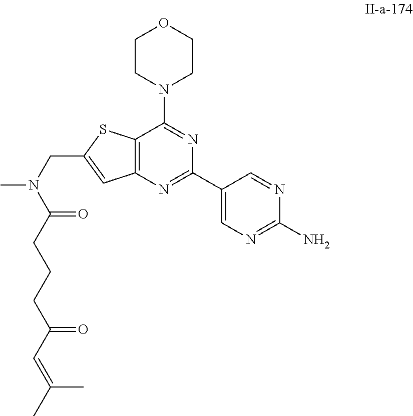

N-((2-(2-aminopyrimidin-5-yl)-4-morpholinothieno[3,2-d]pyrimidin-6-yl)methyl)-N,7-dimethyl-5-oxooct-6-enamide (II-a-174). MS: m/z 510.2 (ES+).

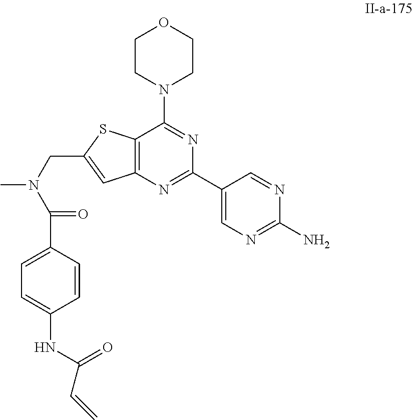

4-acrylamido-N-((2-(2-aminopyrimidin-5-yl)-4-morpholinothieno[3,2-d]pyrimidin-6-yl)methyl)-N-methylbenzamide (II-a-175). MS: m/z 531.2 (ES+).

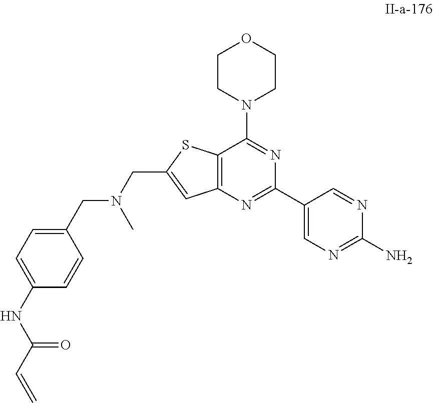

N-(4-((((2-(2-aminopyrimidin-5-yl)-4-morpholinothieno[3,2-d]pyrimidin-6-yl)methyl)(methyl)amino)methyl)phenyl)acrylamide (II-a-176). In a similar fashion, using N-(4-(chloromethyl)phenyl)acrylamide in place of acid, the title compound was prepared. MS: m/z 517.1 (ES+).

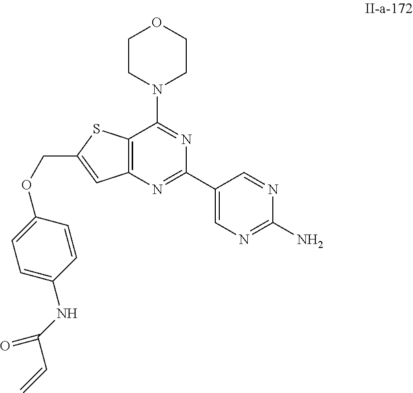

N-(4-((2-(2-aminopyrimidin-5-yl)-4-morpholinothieno[3,2-d]pyrimidin-6-yl)methoxy)phenyl)acrylamide (II-a-172). The title compound was prepared via Mitsunobu reaction by reacting intermediate 6a with N-(4-hydroxyphenyl)acrylamide, followed by Suzuki reaction with 2-aminopyrimidine-5-boronic acid. MS: m/z 490.1 (ES+).

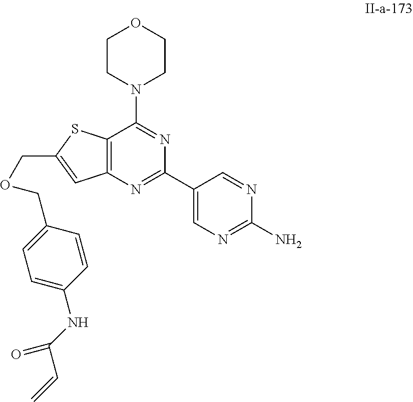

N-(4-(((2-(2-aminopyrimidin-5-yl)-4-morpholinothieno[3,2-d]pyrimidin-6-yl)methoxy)methyl)phenyl)acrylamide (II-a-173). The title compound was prepared via alkylation reaction by reacting intermediate 6a with N-(4-(chloromethyl)phenyl)acrylamide, followed by Suzuki reaction with 2-aminopyrimidine-5-boronic acid. MS: m/z 502.1 (ES+).

### Example 7

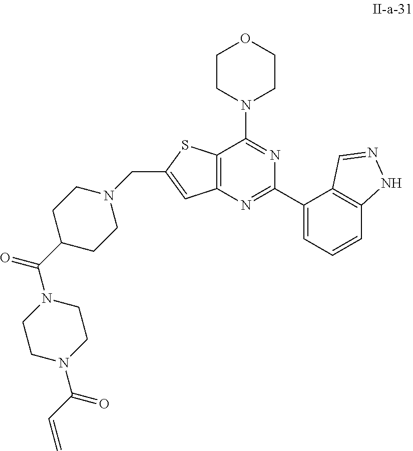

1-(4-(1-((2-(1H-indazol-4-yl)-4-morpholinothieno[3,2-d]pyrimidin-6-yl)methyl)piperidine-4-carbonyl)piperazin-1-yl)prop-2-en-1-one (II-a-31): The title compound was prepared according to the steps and intermediates as described below.

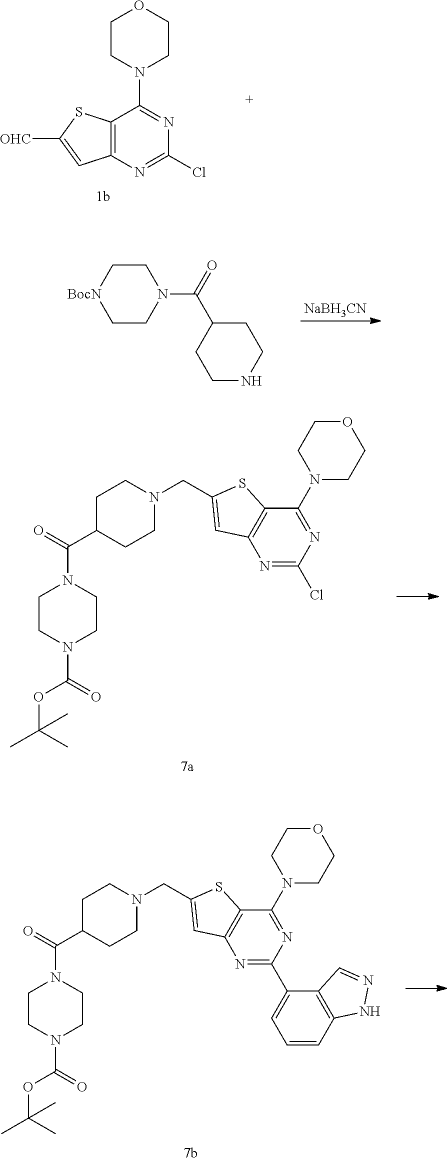

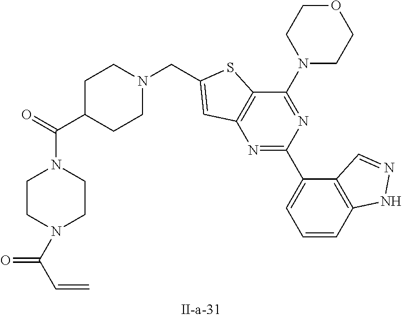

### Step 7a: tert-butyl 4-(1-((2-chloro-4-morpholinothieno[3,2-d]pyrimidin-6-yl)methyl)piperidine-4-carbonyl)piperazine-1-carboxylate (Intermediate 7a)

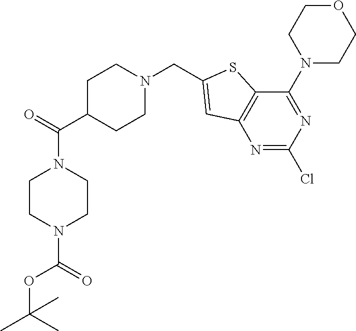

To a suspension of Intermediate 1b (0.2 g, 0.7 mmol) and tert-butyl 4-(piperidine-4-carbonyl)piperazine-1-carboxylate (0.25 g, 0.8 mmol) in DCE (20 mL) was added trimethyl orthoformate (0.22 g, 2.1 mmol) at room temperature under inert atmosphere. The reaction mixture was stirred for 1 h and NaBH(OAc)₃ (0.22 g, 1.06 mmol) was added. After the completion of reaction (monitored by TLC), water was added and extracted with DCM (2×10 mL). The organic layer was washed with water, brine, dried over anhyrous Na₂SO₄ and concentrated in vacuo. The crude compound was purified by column chromatography (5% MeOH/DCM) to afford Intermediate 7a (0.25 g, 64%) as an off white solid. TLC: 10% MeOH/DCM (R_{f}: 0.2); ¹H-NMR (CDCl₃, 200 MHz): δ 7.12 (s, 1H), 3.99 (t, J=4.0 Hz, 4H), 3.90-3.78 (m, 6H), 3.64-3.55 (m, 2H), 3.50-3.38 (m, 6H), 3.10-2.96 (m, 2H), 2.8 (s, 1H), 2.60-2.40 (m, 1H), 2.25-1.85 (m, 4H), 1.75-1.60 (m, 2H), 1.46 (s, 9H); Mass: 565 [M⁺+1]

### Step 7b: tert-butyl 4-((2-(1H-indazol-4-yl)-4-morpholinothieno[3,2-d]pyrimidin-6-yl)methyl)piperidine-4-carbonyl)piperazine-1-carboxylate (Intermediate 7b)

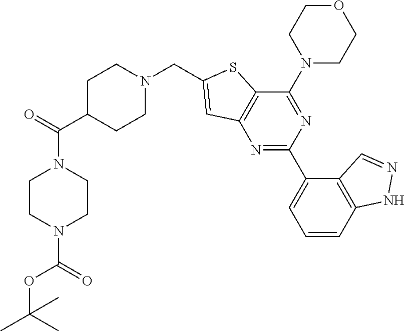

To a stirred solution of Intermediate 7a (0.5 g, 0.8 mmol) in toluene (12.5 mL), EtOH (7.5 mL), H₂O (3.5 mL) was added indazole boronic acid (0.43 g, 1.7 mmol), Na₂CO₃ (0.31 g) and Pd(PPh)₃Cl₂ (0.06 g, 0.09 mmol) at RT. The reaction mixture was degassed with Argon for 1 h and stirred at 140° C. for 16 h. After the completion of reaction (monitored by TLC), the reaction mixture was distributed between DCM and water. The organic layer was separated, dried over anhyrous Na₂SO₄ and concentrated in vacuo. The crude compound was purified by column chromatography (5% MeOH/DCM) to afford Intermediate 7b (0.3 g, 52%) as an off white solid. TLC: 10% MeOH/DCM (R_{f}: 0.3); ¹H-NMR (CDCl₃, 500 MHz): δ 9.0 (s, 1H), 8.27 (d, J=7.0 Hz, 1H), 7.58 (d, J=8 Hz, 1H), 7.50 (t, J=7.5 Hz, 1H), 7.34 (s, 1H), 4.09 (t, J=4.5 Hz, 4H), 3.93 (t, J=4.5 Hz, 4H), 3.85 (s, 2H), 3.6 (bs, 2H), 3.50-3.40 (m, 6H), 3.07 (d, J=11.5 Hz, 2H), 2.5 (t, J=5.0 Hz, 1H), 2.17 (t, J=11.5 Hz, 2H), 2.04-1.94 (m, 2H), 1.70 (d, J=13 Hz, 2H), 1.47 (s, 9H); Mass: 647 [M⁺+1]; MP: 139° C.

### Step 7c: 1-(4-(1-((2-(1H-indazol-4-yl)-4-morpholinothieno[3,2-d]pyrimidin-6-yl)methyl)piperidine-4-carbonyl)piperazin-1-yl)prop-2-en-1-one (II-a-31)

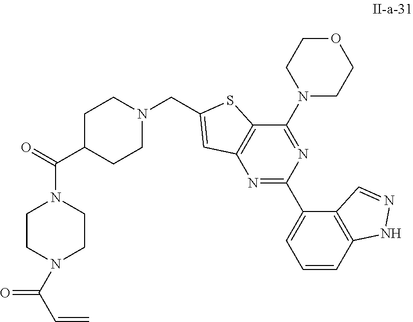

Intermediate 7b was treated with 4N HCl following the procedure described in Example 1, step 1e to afford the de-boc amine HCl salt.

To a stirred solution of the above HCl salt (0.05 g, 0.09 mmol) in DCM (10 mL) was added DIPEA (0.03 g, 0.27 mmol) followed by acryloyl chloride (0.007 g, 0.08 mmol) at −10° C. The reaction mixture was stirred for 1 h at −10° C. After the completion of reaction (monitored by TLC), the reaction was quenched with water and extracted with DCM (2×5 mL). The organic layer was dried over anhyrous Na₂SO₄ and concentrated in vacuo. The crude compound was purified by column chromatography (5% MeOH/DCM) to afford the title compound (0.02 g, 50%) as a grey color solid. TLC: 10% MeOH/DCM (R_{f}: 0.2); ¹H-NMR (CDCl₃, 500 MHz): δ 9.01 (s, 1H), 8.27 (d, J=7.0 Hz, 1H), 7.58 (d, J=8.0 Hz, 1H), 7.5 (t, J=7.5 Hz, 1H), 7.35 (s, 1H), 6.62-6.52 (m, 1H), 6.33 (d, J=16.5 Hz, 1H), 5.76 (d, J=10.5 Hz, 1H), 4.09 (t, J=10.5 Hz, 4H), 3.93 ((t, J=10.5 Hz, 4H), 3.86 (s, 2H), 3.78-3.49 (m, 8H), 3.08 (d, J=11.5 Hz, 2H), 2.58-2.50 (m, 1H), 2.18 (t, J=10.5 Hz, 2H), 2.05-1.95 (m, 2H), 1.71 (d, J=12.5 Hz, 2H); Mass: 601 [M⁺+1].

In similar fashion, using 3-hydroxyphenylboronic acid in step 7b, instead of 4-(4,4,5,5-tetramethyl-1,3,2-dioxaborolan-2-yl)-1H-indazole, (1-((2-(3-hydroxyphenyl)-4-morpholinothieno[3,2-d]pyrimidin-6-yl)methyl)piperidin-4-yl)(4-acryloyl-piperazin-1-yl)methanone (II-a-34) was prepared:

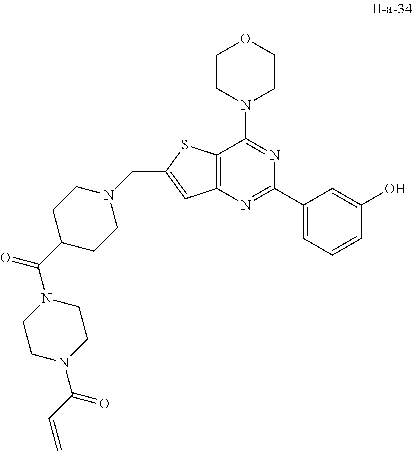

TLC: 10% MeOH/DCM (R_{f}: 0.5); ¹H-NMR (CDCl₃, 500 MHz): δ 8.0 (d, J=8.0 Hz, 1H), 7.91 (s, 1H), 7.33 (t, J=7.5 Hz, 1H), 7.27 (d, J=9.5 Hz, 1H), 6.92 (dd, J=2.0, 7.5 Hz, 1H), 6.54 (dd, J=2.5, 10 Hz, 1H), 6.32 (d, J=16.5 Hz, 1H), 5.73 (d, J=9.5 Hz, 1H), 5.0 (bs, 1H), 4.05 (t, J=4.5 Hz, 4H), 3.89 (t, J=4.5 Hz, 4H), 3.6 (s, 2H), 3.75-3.50 (m, 2H), 3.05 (d, J=11.5 Hz, 2H), 2.58-2.48 (bs, 1H), 2.17 (t, J=11.5 Hz, 2H), 1.97 (q, J=12 Hz, 2H), 1.70 (d, J=12.5 Hz, 2H); Mass: 577 [M⁺+1].

### Example 8

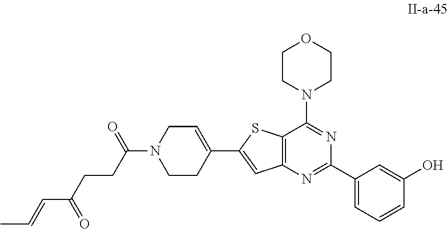

(E)-1-(4-(2-(3-hydroxyphenyl)-4-morpholinothieno[3,2-d]pyrimidin-6-yl)-5,6-dihydropyridin-1(2H)-yl)hept-5-ene-1,4-dione: The title compound was prepared according to the steps and intermediates as described below.

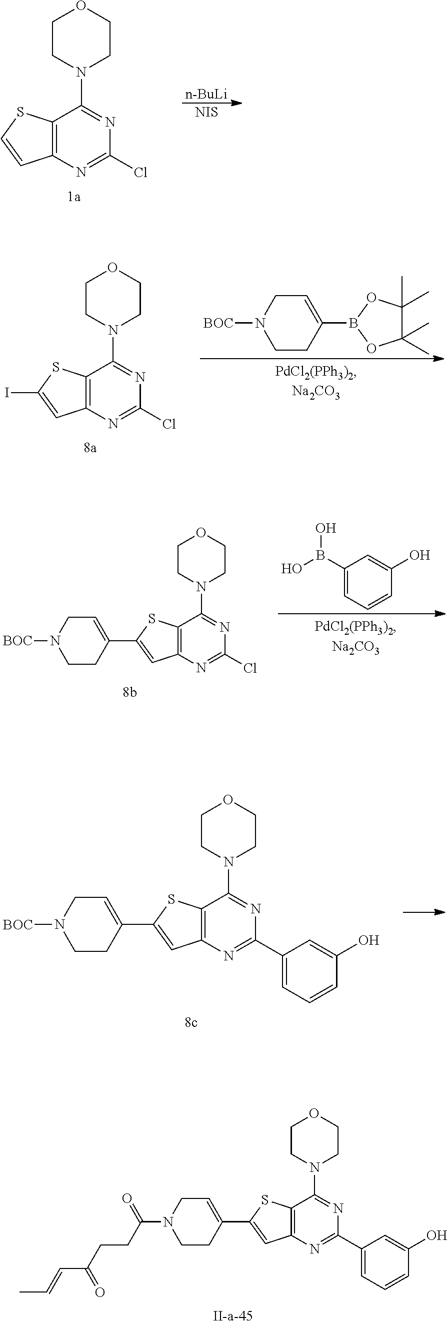

### Step 8a: 4-(2-chloro-6-iodothieno[3,2-d]pyrimidin-4-yl)morpholine (Intermediate 8a)

To a stirred solution of Intermediate 1a (5 g, 0.019 mol) in THF (100 mL) was added n-BuLi (2.5 g, 0.03 mol) at −78° C. over a period of 30 minutes, stirred for 2 h at −40° C. followed by addition of iodine (9.9 g, 0.03 mol) in THF (5 mL) at −78° C. The reaction mixture was stirred for 8 h at RT. After the completion of reaction (monitored by TLC), the reaction was quenched with saturated ammonium chloride (100 mL) and extracted with EtOAc (4×200 mL). The organic layer was washed with sodium thiosulphate solution, dried over anhydrous Na₂SO₄ and concentrated in vacuo. The crude product was washed with diethyl ether to afford intermediate 8b (7 g, 94%) as off white solid. TLC: 30% Ethyl acetate/hexane (R_{f}: 0.3); ¹H-NMR (CDCl₃, 500 MHz): δ 7.57 (s, 1H), 3.94-3.91 (m, 4H), 3.85-3.80 (m, 4H); Mass: 382 [M⁺+1], MP: 173.5° C.

### Step 8b: tert-butyl 4-(2-chloro-4-morpholinothieno[3,2-d]pyrimidin-6-yl)-5,6-dihydropyridine-1(2H)-carboxylate (Intermediate 8b)

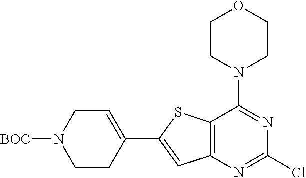

To a stirred solution of 4-(2-chloro-6-iodothieno[3,2-d]pyrimidin-4-yl)morpholine (Intermediate 8a) (0.57 g, 1.5 mmol) in toluene (10 mL), EtOH (6.0 mL), H₂O (3.0 mL) was added tert-butyl 4-(2-chloro-4-morpholinothieno[3,2-d]pyrimidin-6-yl)-5,6-dihydropyridine-1(2H)-carboxylate (0.5 g, 1.6 mmol), Na₂CO₃ (0.7 g) and Pd(PPh)₃Cl₂ (56 mg, 0.08 mmol) at RT. The reaction mixture was degassed with Argon and stirred at 40° C. for 3 h. LC-MS showed the completion of the conversion: MS m/z: 437.1 (M+1). The reaction mixture was used directly for the next step.

### Step 8c: tert-butyl 4-(2-(3-hydroxyphenyl)-4-morpholinothieno[3,2-d]pyrimidin-6-yl)-5,6-dihydropyridine-1(2H)-carboxylate (Intermediate 8c)

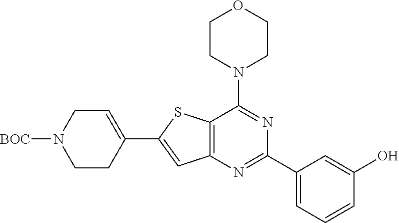

To the reaction mixture from step 8b was added 3-hydroxyphenylboronic acid (0.35 g, 2.5 mmol), Na₂CO₃ (1.0 g) and Pd(PPh)₃Cl₂ (30 mg, 0.04 mmol) at RT. The reaction mixture was degassed with Argon and stirred at 130° C. for 3 h. The reaction was then worked up by adding ethyl acetate 50 ml and washed with water and brine. The organic layer was separated and was dried over Na₂SO₄. After removal of solvent, the crude product was subject to chromatography on silica gel (eluents: EtOAc/hexane 1:1 to 4:1) to give the title compound. MS m/z: 495.1 (M+1).

### Step 8d: (E)-1-(4-(2-(3-hydroxyphenyl)-4-morpholinothieno[3,2-d]pyrimidin-6-yl)-5,6-dihydropyridin-1(2H)-yl)hept-5-ene-1,4-dione (II-a-45)

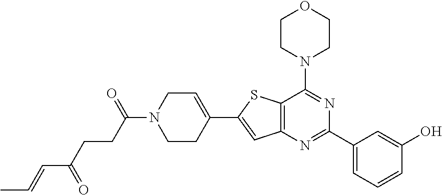

The title compound was prepared by following the procedures described in example 4, step 4b and 4c. MS m/z: 519.1 (M+H⁺).

In the above reaction, when TFA was used for the Boc deprotection, (E)-4-(2-(3-hydroxyphenyl)-4-morpholinothieno[3,2-d]pyrimidin-6-yl)-1-(4-oxohept-5-enoyl)piperidin-3-yl 2,2,2-trifluoroacetate (II-a-46) was obtained as a byproduct:

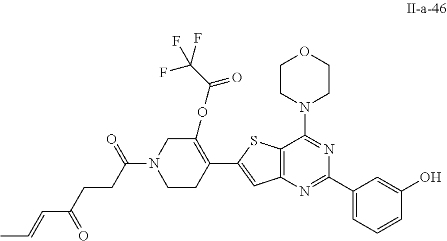

MS m/z: 632.3 (M+H⁺).

The following compounds were prepared by starting with Intermediate 8b and following the procedures or procedure combinations described in previous examples:

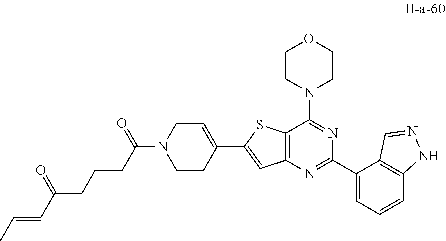

(E)-1-(4-(2-(1H-indazol-4-yl)-4-morpholinothieno[3,2-d]pyrimidin-6-yl)-5,6-dihydropyridin-1(2H)-yl)oct-6-ene-1,5-dione (II-a-60): MS m/z: 557.2 (M+H⁺); ¹H NMR (400 MHz, DMSO-d6): δ: 8.9 (1H s), 8.23 (1H d), 7.67 (1H d), 7.61 (1H, d), 7.48 (1H t), 6.88 (1H, m), 6.51 (1H, dt), 6.11 (1H, dm), 4.19 (2H, bd), 4.02 (4H, bt), 3.84 (4H, bt), 3.7 (2H, m), 2.62 (3H, q), 3.9 (2H, dt), 1.86 (3H, dt), 1.75 (2H, m)

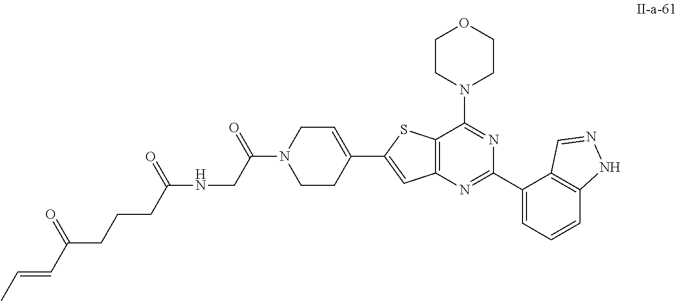

(E)-N-(2-(4-(2-(1H-indazol-4-yl)-4-morpholinothieno[3,2-d]pyrimidin-6-yl)-5,6-dihydropyridin-1(2H)-yl)-2-oxoethyl)-5-oxooct-6-enamide (II-a-61): MS m/z: 614.2 (M+H⁺).

In similar fashion, using a suitable boronic acid in step 8b to couple with intermediate 8a, the following compounds were prepared:

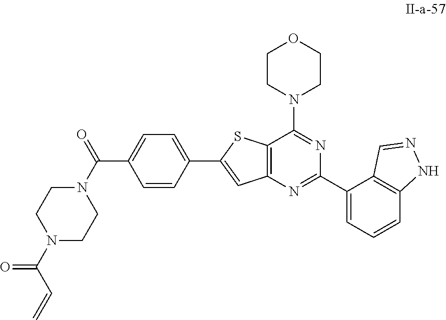

1-(4-(4-(2-(1H-indazol-4-yl)-4-morpholinothieno[3,2-d]pyrimidin-6-yl)benzoyl)piperazin-1-yl)prop-2-en-1-one (II-a-57): MS m/z: 580.2 (M+H⁺).

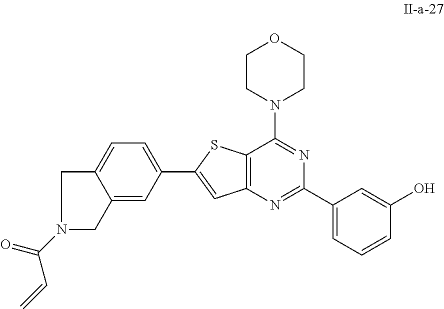

1-(5-(2-(3-hydroxyphenyl)-4-morpholinothieno[3,2-d]pyrimidin-6-yl)isoindolin-2-yl)prop-2-en-1-one (II-a-27): Mass: 485 [M⁺+1].

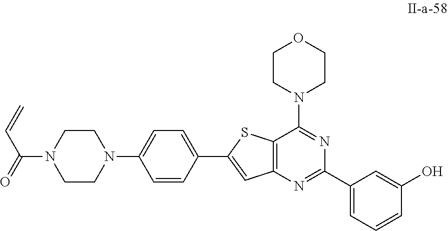

1-(4-(4-(2-(3-hydroxyphenyl)-4-morpholinothieno[3,2-d]pyrimidin-6-yl)phenyl)piperazin-1-yl)prop-2-en-1-one (II-a-58): Mass: 528 [M⁺+1].

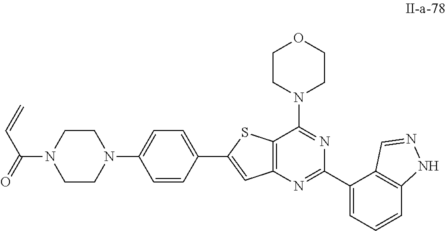

1-(4-(4-(2-(1H-indazol-4-yl)-4-morpholinothieno[3,2-d]pyrimidin-6-yl)phenyl)piperazin-1-yl)prop-2-en-1-one (II-a-78): Mass: 552 [M⁺+1].

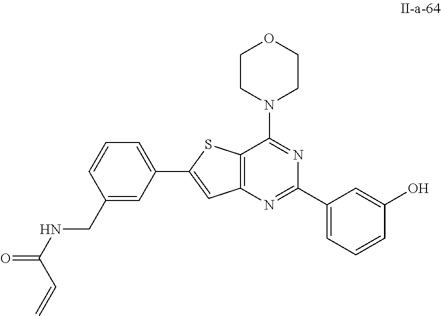

N-(3-(2-(3-hydroxyphenyl)-4-morpholinothieno[3,2-d]pyrimidin-6-yl)benzyl)acrylamide (II-a-64): Mass: 473 [M⁺+1].

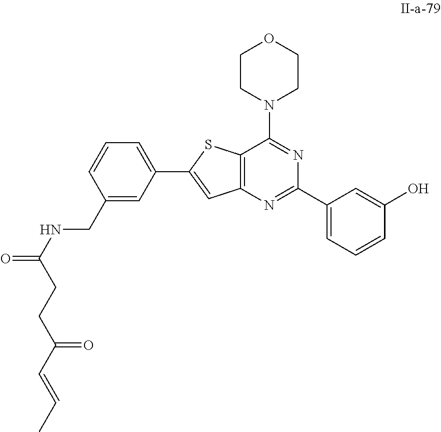

(E)-N-(3-(2-(3-hydroxyphenyl)-4-morpholinothieno[3,2-d]pyrimidin-6-yl)benzyl)-4-oxohept-5-enamide (II-a-79): Mass: 543 [M⁺+1].

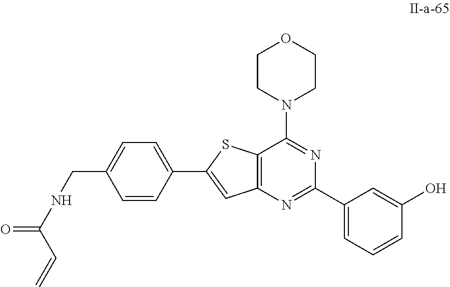

N-(4-(2-(3-hydroxyphenyl)-4-morpholinothieno[3,2-d]pyrimidin-6-yl)benzyl)acrylamide (II-a-65): Mass: 473 [M⁺+1].

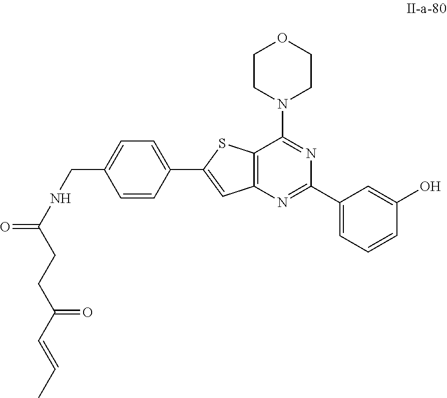

(E)-N-(4-(2-(3-hydroxyphenyl)-4-morpholinothieno[3,2-d]pyrimidin-6-yl)benzyl)-4-oxohept-5-enamide (II-a-80): Mass: 543 [M⁺+1].

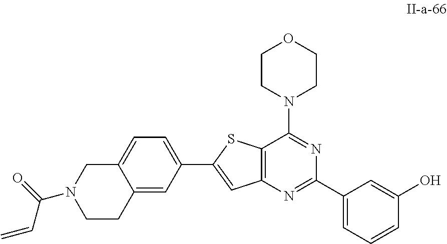

1-(6-(2-(3-hydroxyphenyl)-4-morpholinothieno[3,2-d]pyrimidin-6-yl)-3,4-dihydroisoquinolin-2(1H)-yl)prop-2-en-1-one (II-a-66): Mass: 499 [M⁺+1].

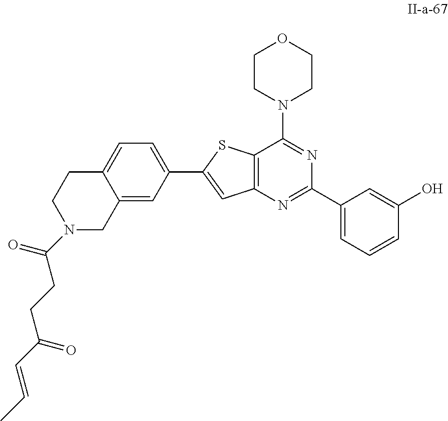

(E)-1-(7-(2-(3-hydroxyphenyl)-4-morpholinothieno[3,2-d]pyrimidin-6-yl)-3,4-dihydroisoquinolin-2(1H)-yl)hept-5-ene-1,4-dione (II-a-67): Mass: 569 [M⁺+1].

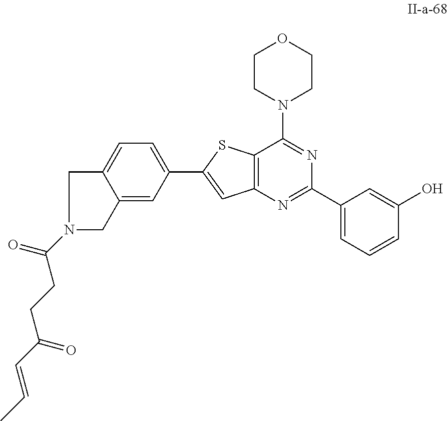

(E)-1-(5-(2-(3-hydroxyphenyl)-4-morpholinothieno[3,2-d]pyrimidin-6-yl)isoindolin-2-yl)hept-5-ene-1,4-dione (II-a-68): Mass: 555 [M⁺+1].

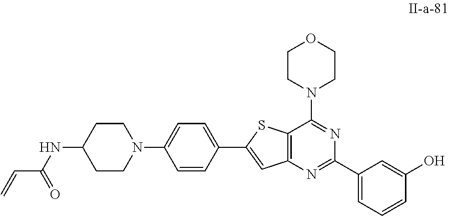

N-(1-(4-(2-(3-hydroxyphenyl)-4-morpholinothieno[3,2-d]pyrimidin-6-yl)phenyl)piperidin-4-yl)acrylamide (II-a-81): Mass: 542 [M⁺+1].

In a similar fashion, using a suitable boronic acid/ester in step 8b, a suitable boronic acid/ester in step 8c, and a suitable carboxylic acid in amide formation (step 8d), the following compounds were prepared:

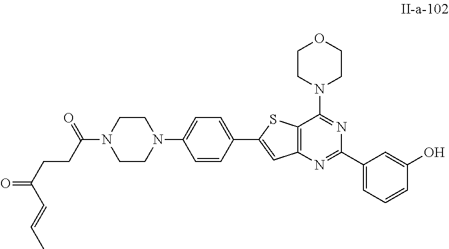

(E)-1-(4-(4-(2-(3-hydroxyphenyl)-4-morpholinothieno[3,2-d]pyrimidin-6-yl)phenyl)piperazin-1-yl)hept-5-ene-1,4-dione (II-a-1021: MS: m/z 598.8 (ES+).

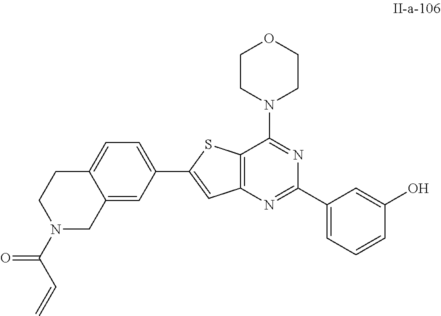

1-(7-(2-(3-hydroxyphenyl)-4-morpholinothieno[3,2-d]pyrimidin-6-yl)-3,4-dihydroisoquinolin-2(1H)-yl)prop-2-en-1-one (II-a-106): MS: m/z 499.0 (ES+).

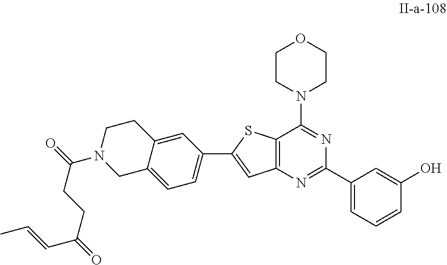

(E)-1-(6-(2-(3-hydroxyphenyl)-4-morpholinothieno[3,2-d]pyrimidin-6-yl)-3,4-dihydroisoquinolin-2(1H)-yl)hept-5-ene-1,4-dione (II-a-108): MS: m/z 569.0 (ES+).

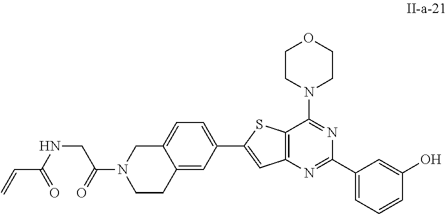

N-(2-(6-(2-(3-hydroxyphenyl)-4-morpholinothieno[3,2-d]pyrimidin-6-yl)-3,4-dihydroisoquinolin-2(1H)-yl)-2-oxoethyl)acrylamide (II-a-121): MS: m/z 556.8 (ES+).

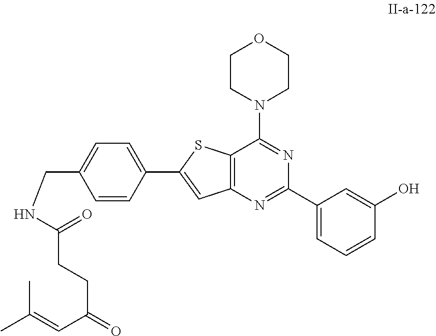

N-(4-(2-(3-hydroxyphenyl)-4-morpholinothieno[3,2-d]pyrimidin-6-yl)benzyl)-6-methyl-4-oxohept-5-enamide (II-a-122): MS: m/z 539.2 (ES+).

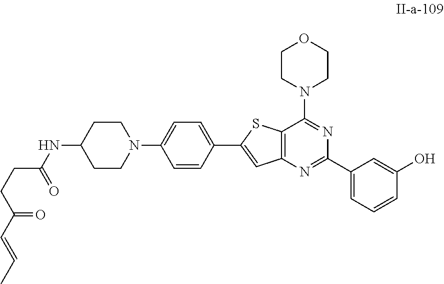

(E)-N-(1-(4-(2-(3-hydroxyphenyl)-4-morpholinothieno[3,2-d]pyrimidin-6-yl)phenyl)piperidin-4-yl)-4-oxohept-5-enamide (II-a-109): MS: m/z 612.8 (ES+).

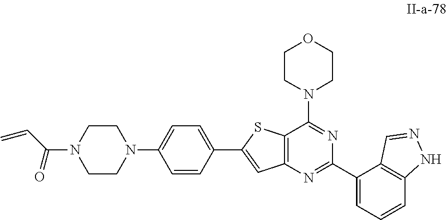

1-(4-(4-(2-(1H-indazol-4-yl)-4-morpholinothieno[3,2-d]pyrimidin-6-yl)phenyl)piperazin-1-yl)prop-2-en-1-one (II-a-78): MS: m/z 552.7 (ES+).

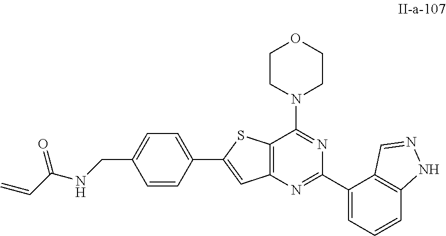

N-(4-(2-(1H-indazol-4-yl)-4-morpholinothieno[3,2-d]pyrimidin-6-yl)benzyl)acrylamide (II-a-107): MS: m/z 497.7 (ES+).

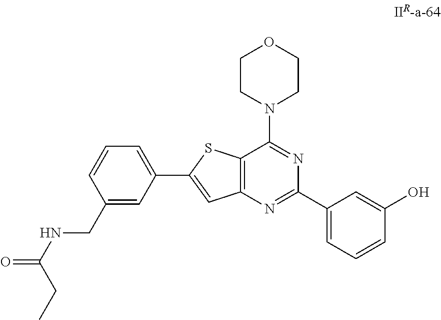

N-(3-(2-(3-hydroxyphenyl)-4-morpholinothieno[3,2-d]pyrimidin-6-yl)benzyl)propionamide (II^{R}-a-64): MS: m/z 475.1 (ES+).

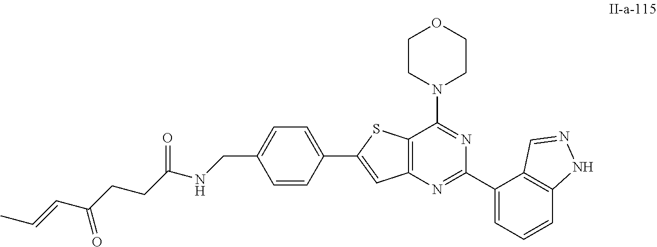

(E)-N-(4-(2-(1H-indazol-4-yl)-4-morpholinothieno[3,2-d]pyrimidin-6-yl)benzyl)-4-oxohept-5-enamide (II-a-115): MS: m/z 567.7 (ES+).

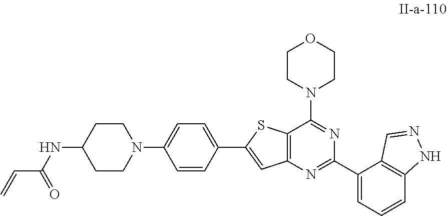

N-(1-(4-(2-(1H-indazol-4-yl)-4-morpholinothieno[3,2-d]pyrimidin-6-yl)phenyl)piperidin-4-yl)acrylamide (II-a-110): MS: m/z 566.8 (ES+).

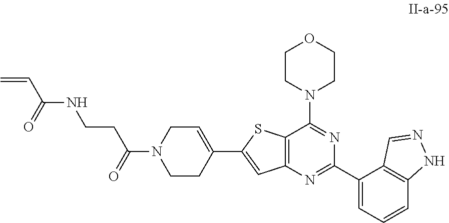

N-(3-(4-(2-(1H-indazol-4-yl)-4-morpholinothieno[3,2-d]pyrimidin-6-yl)-5,6-dihydropyridin-1(2H)-yl)-3-oxopropyl)acrylamide (II-a-95): MS: m/z 544.2 (ES+).

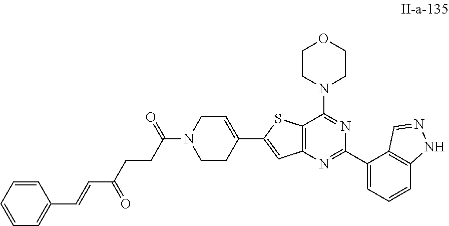

(E)-1-(4-(2-(1H-indazol-4-yl)-4-morpholinothieno[3,2-d]pyrimidin-6-yl)-5,6-dihydropyridin-1(2H)-yl)-6-phenylhex-5-ene-1,4-dione (II-a-135): MS: m/z 605.3 (ES+).

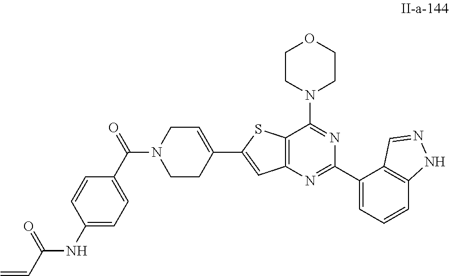

N-(4-(4-(2-(1H-indazol-4-yl)-4-morpholinothieno[3,2-d]pyrimidin-6-yl)-1,2,3,6-tetrahydropyridine-1-carbonyl)phenyl)acrylamide (II-a-144): MS: m/z 592.1 (ES+).

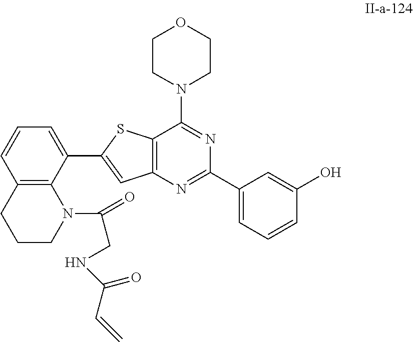

N-(2-(8-(2-(3-hydroxyphenyl)-4-morpholinothieno[3,2-d]pyrimidin-6-yl)-3,4-dihydroquinolin-1(2H)-yl)-2-oxoethyl)acrylamide (II-a-124): MS: m/z 556.1 (ES+).

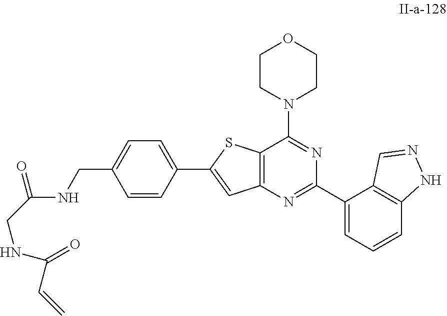

N-(2-(4-(2-(1H-indazol-4-yl)-4-morpholinothieno[3,2-d]pyrimidin-6-yl)benzylamino)-2-oxoethyl)acrylamide (II-a-128): MS: m/z.

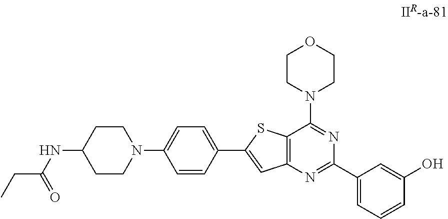

N-(1-(4-(2-(3-hydroxyphenyl)-4-morpholinothieno[3,2-d]pyrimidin-6-yl)phenyl)piperidin-4-yl)propionamide (II^{R}-a-81): The title compound was prepared by hydrogenation of II-a-81 with 5% Pd/C in MeOH under hydrogen. MS: m/z 544.2 (ES+).

In a similar fashion, using 2-amino-pyrimidine-4-boronic acid in place of indazole-4-boronic acid for the Suzuki coupling step (step 6e), the following compounds were prepared:

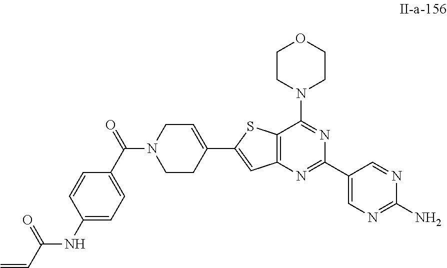

N-(4-(4-(2-(2-aminopyrimidin-5-yl)-4-morpholinothieno[3,2-d]pyrimidin-6-yl)-1,2,3,6-tetrahydropyridine-1-carbonyl)phenyl)acrylamide (II-a-156). MS: m/z 569.2 (ES+).

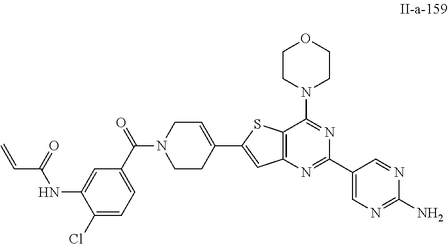

N-(5-(4-(2-(2-aminopyrimidin-5-yl)-4-morpholinothieno[3,2-d]pyrimidin-6-yl)-1,2,3,6-tetrahydropyridine-1-carbonyl)-2-chlorophenyl)acrylamide (II-a-159). MS: m/z 603.0 (ES+).

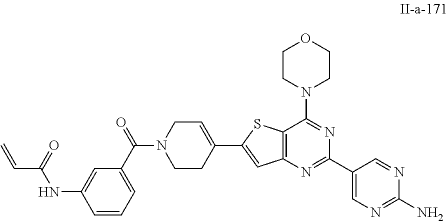

N-(3-(4-(2-(2-aminopyrimidin-5-yl)-4-morpholinothieno[3,2-d]pyrimidin-6-yl)-1,2,3,6-tetrahydropyridine-1-carbonyl)phenyl)acrylamide (II-a-171). MS: m/z 569.2 (ES+).

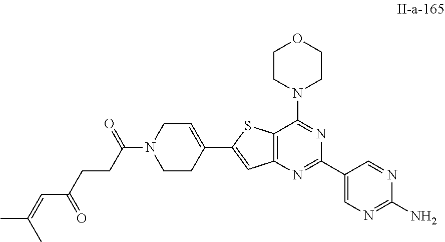

1-(4-(2-(2-aminopyrimidin-5-yl)-4-morpholinothieno[3,2-d]pyrimidin-6-yl)-5,6-dihydropyridin-1(2H)-yl)-6-methylhept-5-ene-1,4-dione (II-a-165). MS: m/z 534.2 (ES+).

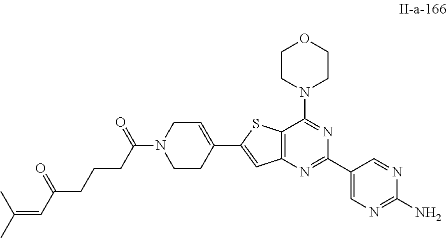

1-(4-(2-(2-aminopyrimidin-5-yl)-4-morpholinothieno[3,2-d]pyrimidin-6-yl)-5,6-dihydropyridin-1(2H)-yl)-7-methyloct-6-ene-1,5-dione (II-a-166). MS: m/z 548.2 (ES+).

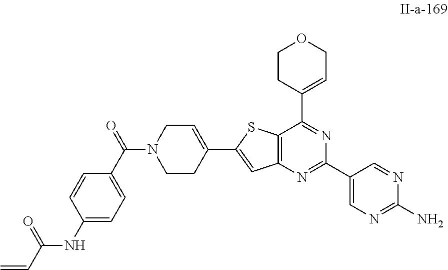

N-(4-(4-(2-(2-aminopyrimidin-5-yl)-4-(3,6-dihydro-2H-pyran-4-yl)thieno[3,2-d]pyrimidin-6-yl)-1,2,3,6-tetrahydropyridine-1-carbonyl)phenyl)acrylamide (II-a-169). The title compound was prepared in a similar way as to II-a-165, by using 2-(3,6-dihydro-2H-pyran-4-yl)-4,4,5,5-tetramethyl-1,3,2-dioxaborolane in Suzuki coupling instead of Cl-displacement reaction with morpholine at the very beginning MS: m/z 545.2 (ES+).

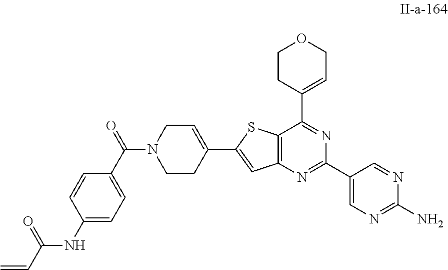

N-(4-(4-(2-(2-aminopyrimidin-5-yl)-4-(3,6-dihydro-2H-pyran-4-yl)thieno[3,2-d]pyrimidin-6-yl)-1,2,3,6-tetrahydropyridine-1-carbonyl)phenyl)acrylamide (II-a-164). The title compound was prepared in a similar way as to II-a-156, by using 2-(3,6-dihydro-2H-pyran-4-yl)-4,4,5,5-tetramethyl-1,3,2-dioxaborolane in Suzuki coupling instead of Cl-displacement reaction with morpholine at the very beginning MS: m/z 566.2 (ES+).

### Example 9

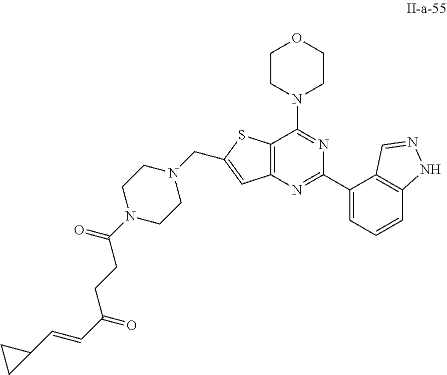

(E)-1-(4-((2-(1H-indazol-4-yl)-4-morpholinothieno[3,2-d]pyrimidin-6-yl)methyl)piperazin-1-yl)-6-cyclopropylhex-5-ene-1,4-dione (II-a-55): The title compound was prepared according to the steps and intermediates as described below.

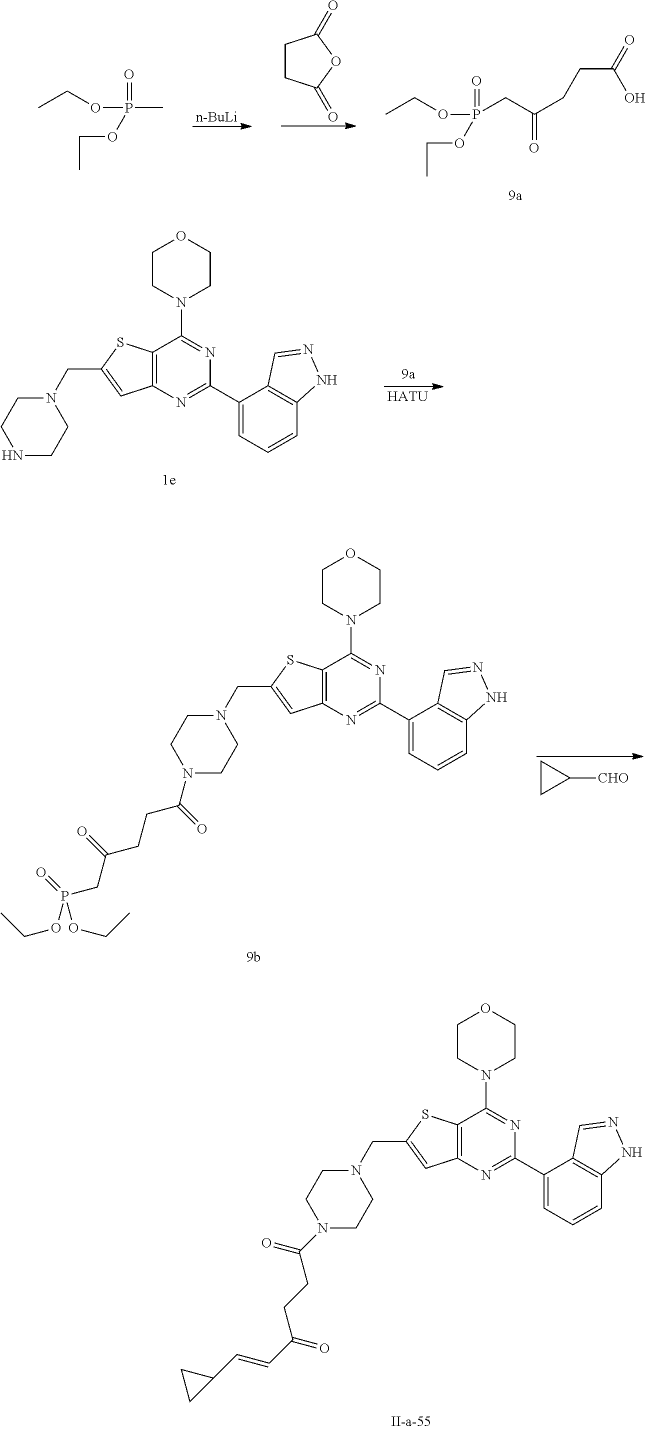

### Step 9a: 5-(diethoxyphosphoryl)-4-oxopentanoic acid (Intermediate 9a)

To a solution of diethyl methylphosphonate (0.76 g, 5.0 mmol) in 20 ml THF at −78° C. was added n-BuLi (2.5 N, 5.0 mmol) slowly. The reaction mixture was stirred at −78° C. for 1 h. Succinic anhydride (0.50 g 5.0 mmol) in 5.0 ml of anhydrous THF was introduced into the reaction at −78° C. slowly. The reaction mixture was stirred for 1 h at −78° C. 1 N HCl (5.0 ml) aqueous solution was added and the mixture was warmed up to RT. The THF was then removed under vacuum and the remaining aqueous was extracted by DCM (3×10 mL). The organic layer was dried over Na₂SO₄, filtered and the solvent was removed. The residue was purified by chromatography on silica gel (eluents: EtOAc/MeOH 20:1) to provide the acid 9a. MS m/z: 253.1 (M+1); ¹H NMR (400 MHz, CDCl₃): δ: 4.15 (4H m), 3.18 (1H s), 3.13 (1H s), 2.95 (2H t, J=6.44 Hz), 2.63 (2H t, J=6.40 Hz), 1.33 (6H m).

### Step 9b: Diethyl 5-(4-((2-(1H-indazol-4-yl)-4-morpholinothieno[3,2-d]pyrimidin-6-yl)methyl)piperazin-1-yl)-2,5-dioxopentylphosphonate (Intermediate 9b)

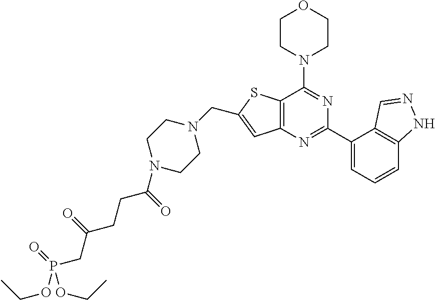

The title compound was obtained by coupling the acid 9a and intermediate 1e (from Example 1) using HATU following the procedure described in step 1f. MS m/z: 670.3 (M+1).

### Step 9c: (E)-1-(4-((2-(1H-indazol-4-yl)-4-morpholinothieno[3,2-d]pyrimidin-6-yl)methyl)piperazin-1-yl)-6-cyclopropylhex-5-ene-1,4-dione (II-a-55)

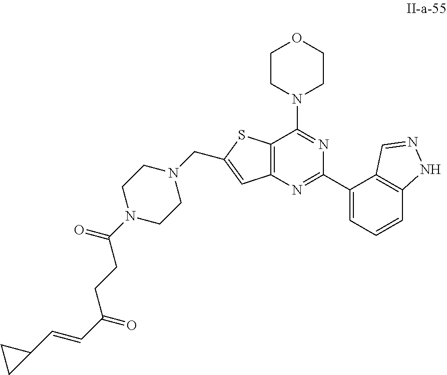

To a solution of Intermediate 9b (25 mg, 0.04 mmol) and cyclopropanecarbaldehyde (28 mg, 0.4 mmol) in THF/H2O (1.5 ml/1.0 ml) was added Na₂CO₃ (25 mg, 0.25 mmol) at RT. The reaction mixture was stirred for 10 hours and was quenched by 1N HCl to PH˜5. The crude residue was purified by prep. HPLC (25% to 90% CH₃CN aqueous containing 0.1% TFA) to give 10.0 mg of the title compound as a TFA salt. MS m/z: 586.2 (M+1); ¹H NMR (400 MHz, CDCl₃, MeOD): δ: 8.41 (1H d, J=0.88 Hz), 7.83 (1H d, J=6.84 Hz), 7.61 (1H d, J=8.24 Hz), 7.44 (1H, s), 7.38 (1H t, J=7.32 Hz), 6.21 (1H dd, J=10.1, 15.6 Hz), 6.06 (1H d, 15.6 Hz), 3.79 (8H, m), 3.56 (4H, m), 2.69 (6H, m), 2.43 (3H, m), 0.83 (2H, m), 0.51 (2H, m).

In similar fashion, treating Intermediate 9b with appropriate aldehydes, the following compounds were prepared:

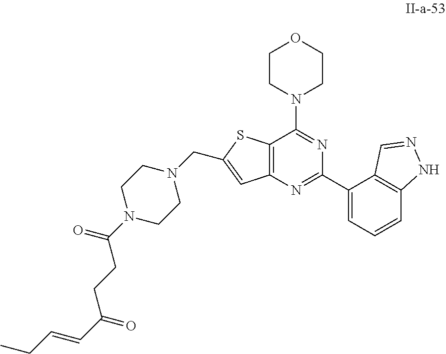

(E)-1-(4-((2-(1H-indazol-4-yl)-4-morpholinothieno[3,2-d]pyrimidin-6-yl)methyl)piperazin-1-yl)oct-5-ene-1,4-dione (II-a-53): MS m/z: 574.3 (M+1). ¹H NMR (400 MHz, CDCl₃, MeOD): δ: 8.76 (1H d, J=0.92 Hz), 8.07 (1H d, J=7.32 Hz), 7.53 (1H d, J=8.24 Hz), 7.40 (1H dd, J=7.36 Hz, 8.28 Hz), 7.30 (1H, s), 6.88 (1H dt, J=6.4 Hz, 16.04 Hz), 6.04 (1H d, 16.04 Hz), 4.01 (4H m), 3.84 (4H m), 3.79 (2H, m), 3.52 (2H, m), 2.83 (2H, m), 2.51 (6H, m), 2.16 (2H, m), 0.99 (3H, t, J=7.32 Hz).

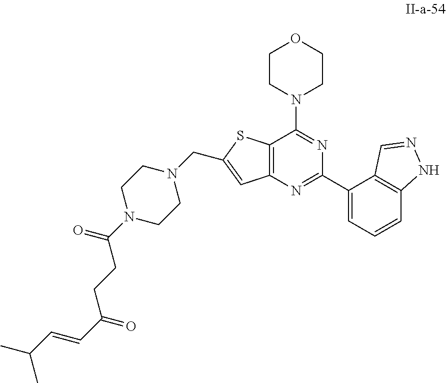

(E)-1-(4-((2-(1H-indazol-4-yl)-4-morpholinothieno[3,2-d]pyrimidin-6-yl)methyl)piperazin-1-yl)-7-methyloct-5-ene-1,4-dione (II-a-54): MS m/z: 588.1 (M+1).

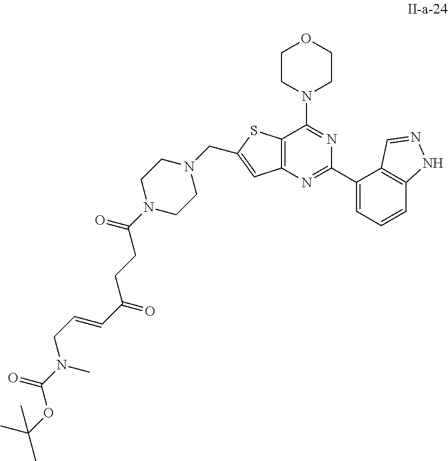

(E)-tert-butyl 7-(4-((2-(1H-indazol-4-yl)-4-morpholinothieno[3,2-d]pyrimidin-6-yl)methyl)piperazin-1-yl)-4,7-dioxohept-2-enylmethyl)carbamates (II-a-24): MS m/z: 689.3 (M+1).

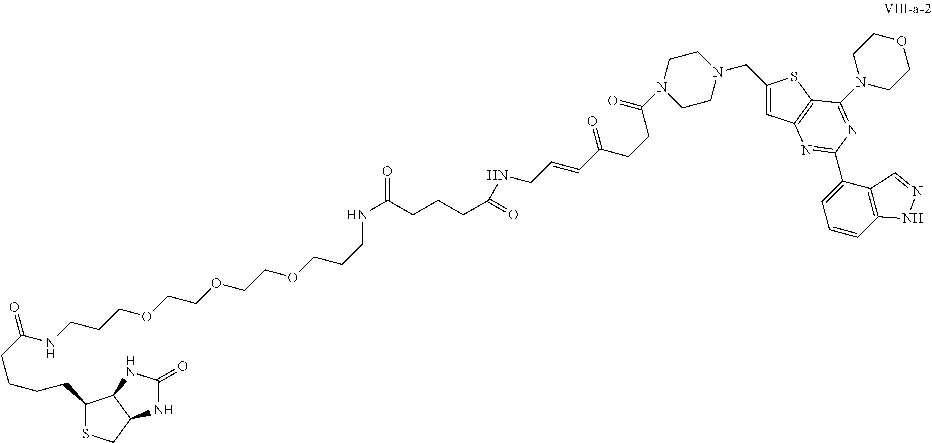

N1-((E)-7-(4-((2-(1H-indazol-4-yl)-4-morpholinothieno[3,2-d]pyrimidin-6-yl)methyl)piperazin-1-yl)-4,7-dioxohept-2-enyl)-N5-(15-oxo-19-((3aS,4S,6aR)-2-oxohexahydro-1H-thieno[3,4-d]imidazol-4-yl)-4,7,10-trioxa-14-azanonadecyl)glutaramide (VIII-a-2): MS m/z: 1117.5 (M+1).

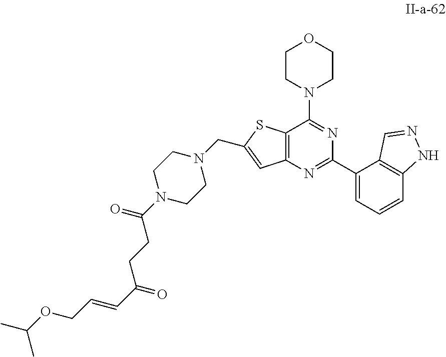

(E)-1-(4-((2-(1H-indazol-4-yl)-4-morpholinothieno[3,2-d]pyrimidin-6-yl)methyl)piperazin-1-yl)-7-isopropoxyhept-5-ene-1,4-dione (II-a-62): MS m/z: 618.3 (M+1). ¹H NMR (400 MHz, CDCl₃, MeOD): δ: 8.57 (1H, s), 8.03 (1H d, J=7.36 Hz), 7.63 (1H d, J=8.24 Hz), 7.56 (1H, s), 7.44 (1H, t, J=7.80 Hz), 6.81 (1H, dt, J=6.34 Hz, 16.04 Hz), 6.27 (1H dt, J=2.06 Hz, 16.04 Hz), 4.11 (8H, m), 3.86 (4H, m), 3.7-3.6 (5H, m), 2.87 (4H, m), 2.75 (2H, m), 2.55 (2H, m), 1.09 (6H, d, J=5.96 Hz).

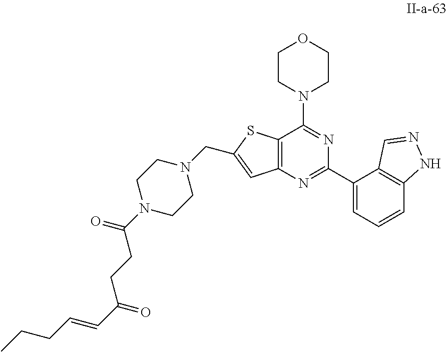

(E)-1-(4-((2-(1H-indazol-4-yl)-4-morpholinothieno[3,2-d]pyrimidin-6-yl)methyl)piperazin-1-yl)non-5-ene-1,4-dione (II-a-63): MS m/z: 588.3 (M+1). ¹H NMR (400 MHz, CDCl₃, MeOD): δ: 8.61 (1H, s), 8.04 (1H d, J=7.36 Hz), 7.61 (1H d, J=8.24 Hz), 7.52 (1H, s), 7.44 (1H, t, J=7.80 Hz), 6.82 (1H, dt, J=6.88 Hz, 16.04 Hz), 6.03 (1H d, J=16.04 Hz), 4.08 (6H, m), 3.86 (4H, m), 3.63 (4H, m), 2.84 (2H, m), 2.78 (2H, m), 2.69 (2H, m), 2.54 (2H, m), 2.12 (2H, m), 1.39 (2H, m), 0.83 (3H, t).

In similar fashion, treating Intermediate 9b with appropriate ketone at 40-60° C., 1-(4-((2-(1H-indazol-4-yl)-4-morpholinothieno[3,2-d]pyrimidin-6-yl)methyl)piperazin-1-yl)-5-cyclobutylidenepentane-1,4-dione (II-a-82) was prepared:

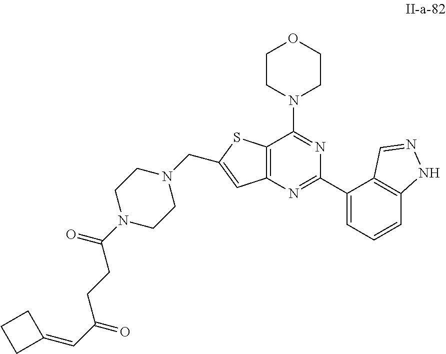

MS m/z: 586.1 (M+1).

In a similar fashion, using appropriate aldehydes or ketones, the following compounds were prepared:

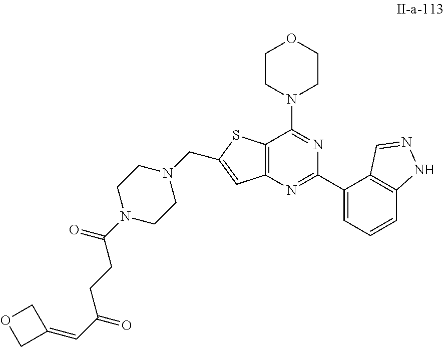

1-(4-((2-(1H-indazol-4-yl)-4-morpholinothieno[3,2-d]pyrimidin-6-yl)methyl)piperazin-1-yl)-5-(oxetan-3-ylidene)pentane-1,4-dione (II-a-113): MS: m/z 588.1 (ES+).

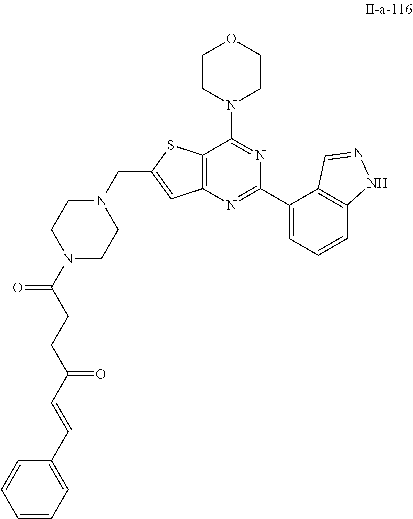

(E)-1-(4-((2-(1H-indazol-4-yl)-4-morpholinothieno[3,2-d]pyrimidin-6-yl)methyl)piperazin-1-yl)-6-phenylhex-5-ene-1,4-dione (II-a-116): MS: m/z 622.2 (ES+).

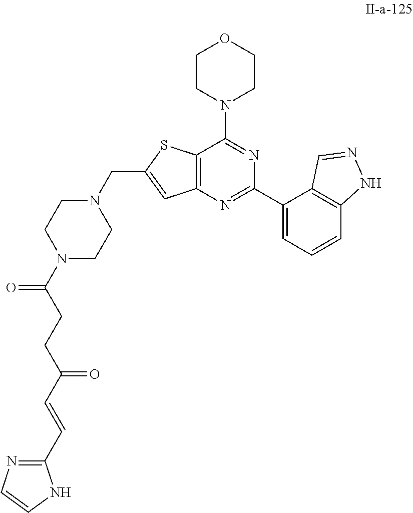

(E)-1-(4-((2-(1H-indazol-4-yl)-4-morpholinothieno[3,2-d]pyrimidin-6-yl)methyl)piperazin-1-yl)-6-(1H-imidazol-2-yl)hex-5-ene-1,4-dione (II-a-125): MS: m/z 612.2 (ES+)

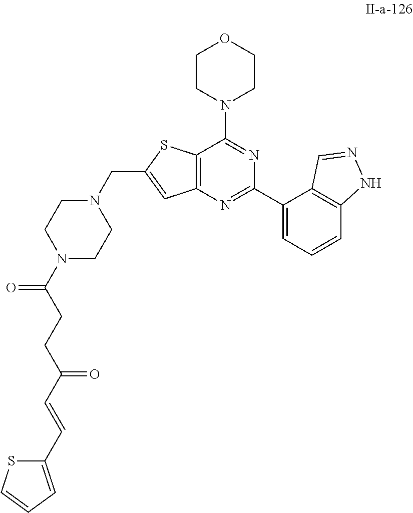

(E)-1-(4-((2-(1H-indazol-4-yl)-4-morpholinothieno[3,2-d]pyrimidin-6-yl)methyl)piperazin-1-yl)-6-(thiophen-2-yl)hex-5-ene-1,4-dione (II-a-126): MS: m/z 628.3 (ES+).

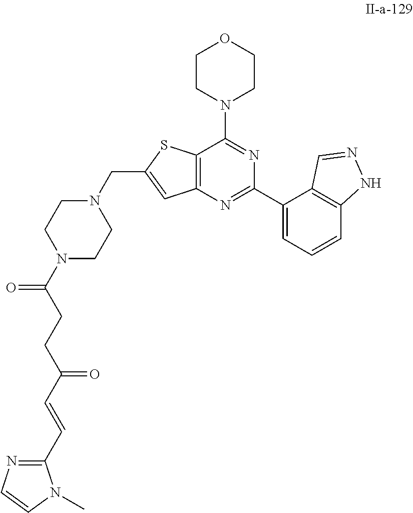

(E)-1-(4-((2-(1H-indazol-4-yl)-4-morpholinothieno[3,2-d]pyrimidin-6-yl)methyl)piperazin-1-yl)-6-(1-methyl-1H-imidazol-2-yl)hex-5-ene-1,4-dione (II-a-129): MS: m/z 626.3 (ES+).

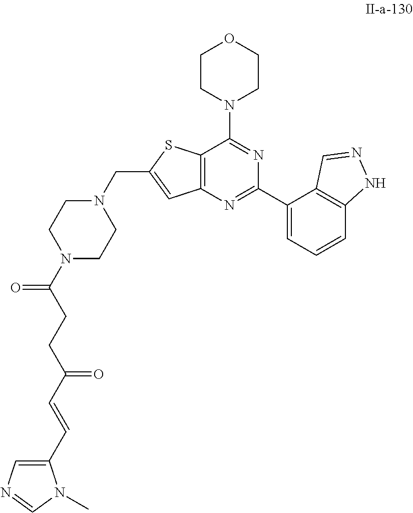

(E)-1-(4-((2-(1H-indazol-4-yl)-4-morpholinothieno[3,2-d]pyrimidin-6-yl)methyl)piperazin-1-yl)-6-(1-methyl-1H-imidazol-5-yl)hex-5-ene-1,4-dione (II-a-130): MS: m/z 626.3 (ES+).

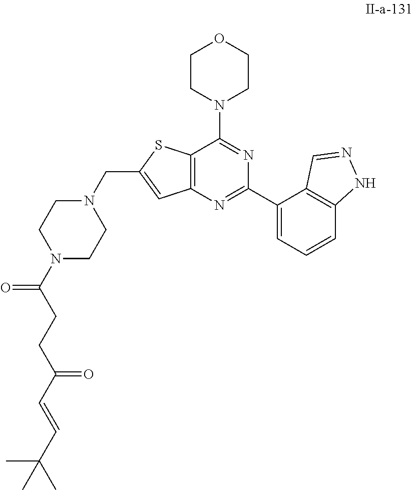

(E)-1-(4-((2-(1H-indazol-4-yl)-4-morpholinothieno[3,2-d]pyrimidin-6-yl)methyl)piperazin-1-yl)-7,7-dimethyloct-5-ene-1,4-dione (II-a-131): MS: m/z 602.3 (ES+).

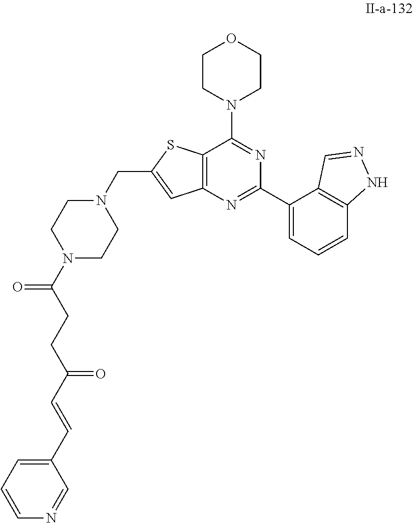

(E)-1-(4-((2-(1H-indazol-4-yl)-4-morpholinothieno[3,2-d]pyrimidin-6-yl)methyl)piperazin-1-yl)-6-(pyridin-3-yl)hex-5-ene-1,4-dione (II-a-132): MS: m/z 623.3 (ES+).

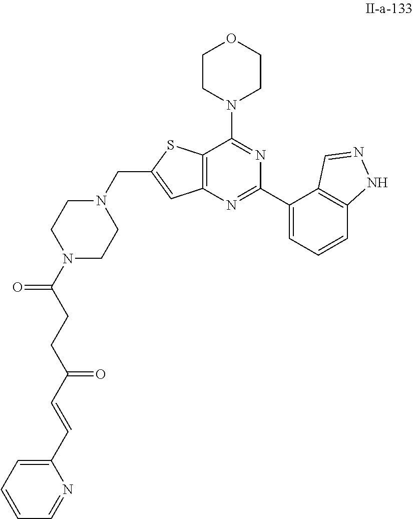

(E)-1-(4-((2-(1H-indazol-4-yl)-4-morpholinothieno[3,2-d]pyrimidin-6-yl)methyl)piperazin-1-yl)-6-(pyridin-2-yl)hex-5-ene-1,4-dione (II-a-133): MS: m/z 623.3 (ES+).

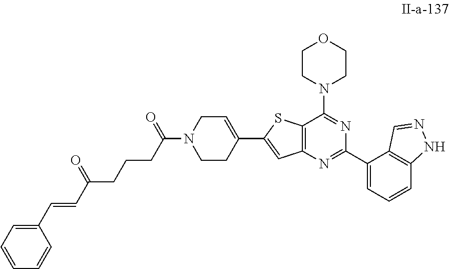

(E)-1-(4-(2-(1H-indazol-4-yl)-4-morpholinothieno[3,2-d]pyrimidin-6-yl)-5,6-dihydropyridin-1(2H)-yl)-7-phenylhept-6-ene-1,5-dione (II-a-137): MS: m/z 619.2 (ES+)

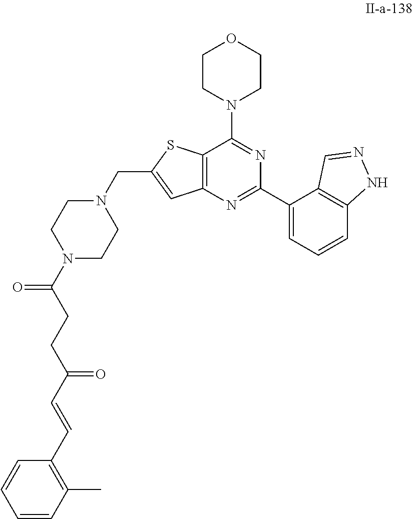

(E)-1-(4-((2-(1H-indazol-4-yl)-4-morpholinothieno[3,2-d]pyrimidin-6-yl)methyl)piperazin-1-yl)-6-o-tolylhex-5-ene-1,4-dione (II-a-138): MS: m/z 636.3 (ES+)

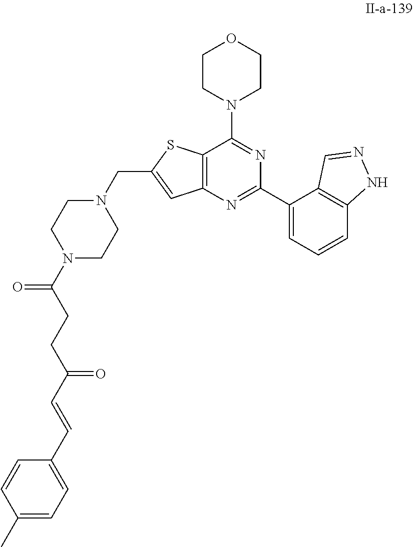

(E)-1-(4-((2-(1H-indazol-4-yl)-4-morpholinothieno[3,2-d]pyrimidin-6-yl)methyl)piperazin-1-yl)-6-p-tolylhex-5-ene-1,4-dione (II-a-139): MS: m/z 636.3 (ES+)

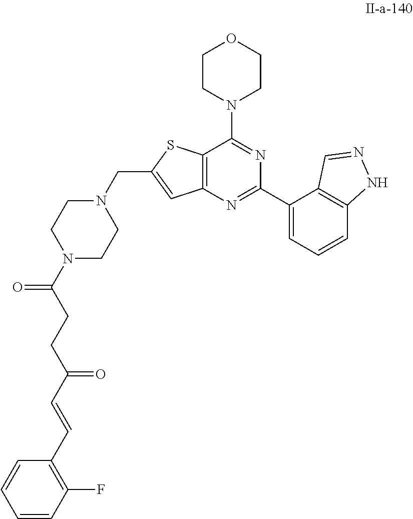

(E)-1-(4-((2-(1H-indazol-4-yl)-4-morpholinothieno[3,2-d]pyrimidin-6-yl)methyl)piperazin-1-yl)-6-(2-fluorophenyl)hex-5-ene-1,4-dione (II-a-140): MS: m/z 640.3 (ES+).

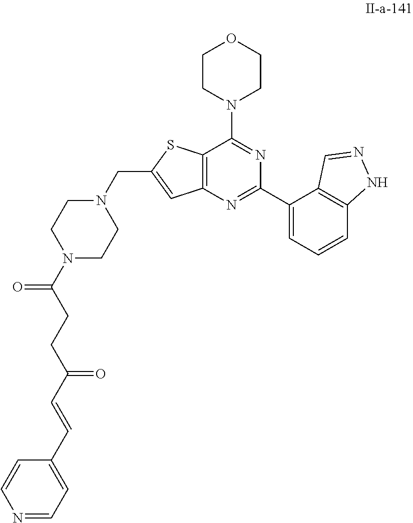

(E)-1-(4-((2-(1H-indazol-4-yl)-4-morpholinothieno[3,2-d]pyrimidin-6-yl)methyl)piperazin-1-yl)-6-(pyridin-4-yl)hex-5-ene-1,4-dione (II-a-141): MS: m/z 623.3 (ES+)

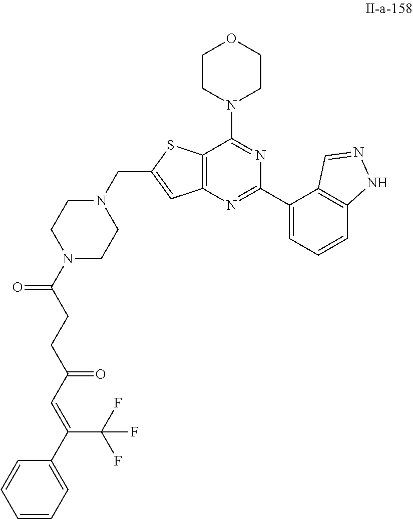

(Z)-1-(4-((2-(1H-indazol-4-yl)-4-morpholinothieno[3,2-d]pyrimidin-6-yl)methyl)piperazin-1-yl)-7,7,7-trifluoro-6-phenylhept-5-ene-1,4-dione (II-a-158). MS: m/z 690.2 (ES+).

In a similar fashion, using diethyl ethylphosphonate in step 9a and appropriate aldehydes in final condensation step, the following compounds were prepared:

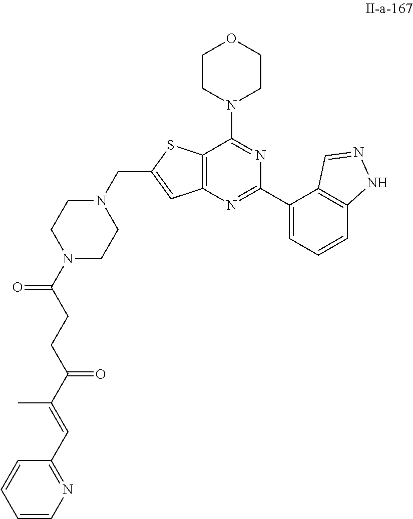

(E)-1-(4-((2-(1H-indazol-4-yl)-4-morpholinothieno[3,2-d]pyrimidin-6-yl)methyl)piperazin-1-yl)-5-methyl-6-(pyridin-2-yl)hex-5-ene-1,4-dione (II-a-167). MS: m/z 637.0 (ES+).

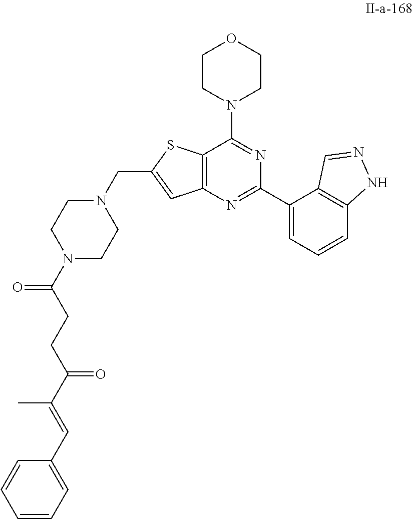

(E)-1-(4-((2-(1H-indazol-4-yl)-4-morpholinothieno[3,2-d]pyrimidin-6-yl)methyl)piperazin-1-yl)-5-methyl-6-phenylhex-5-ene-1,4-dione (II-a-168). MS: m/z 636.0 (ES+).

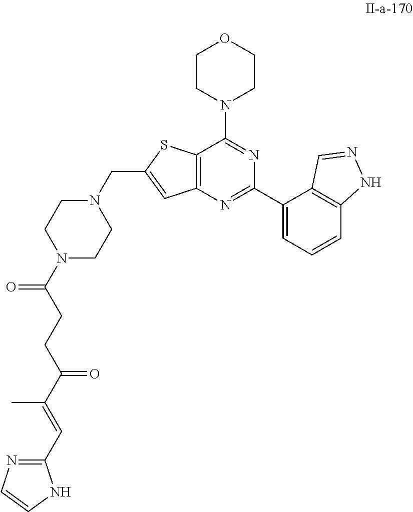

(E)-1-(4-((2-(1H-indazol-4-yl)-4-morpholinothieno[3,2-d]pyrimidin-6-yl)methyl)piperazin-1-yl)-6-(1H-imidazol-2-yl)-5-methylhex-5-ene-1,4-dione (II-a-170). MS: m/z 626.0 (ES+).

### Example 10

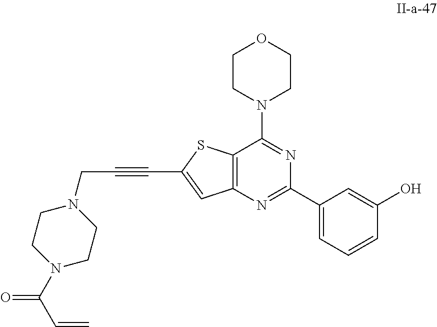

1-(4-(3-(2-(3-hydroxyphenyl)-4-morpholinothieno[3,2-d]pyrimidin-6-yl)prop-2-ynyl)piperazin-1-yl)prop-2-en-1-one (II-a-47): The title compound was prepared according to the steps and intermediates as described below.

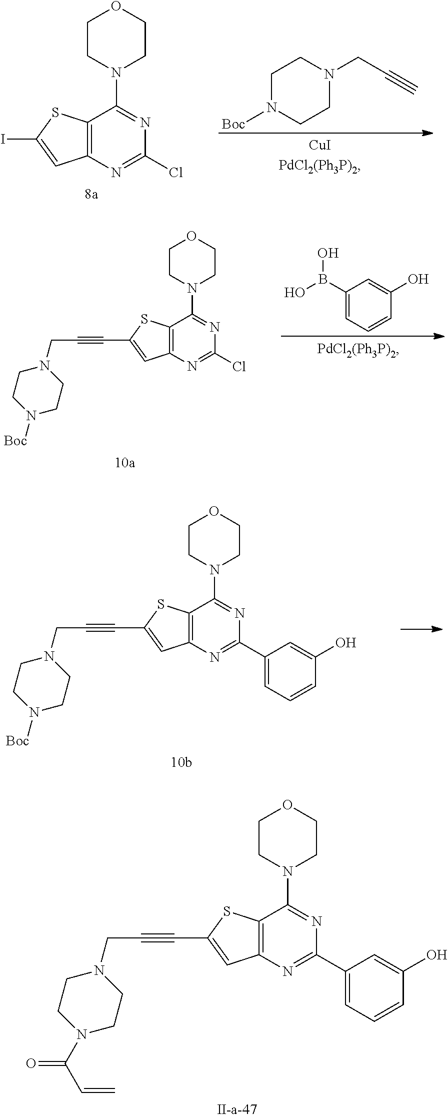

### Step 10a: tert-butyl 4-(3-(2-chloro-4-morpholinothieno[3,2-d]pyrimidin-6-yl)prop-2-ynyl)piperazine-1-carboxylate (Intermediate 10a)

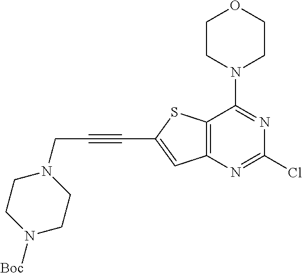

To a stirred solution of Intermediate 8a (1.0 g, 2.6 mmol), tert-butyl 4-(prop-2-ynyl)piperazine-1-carboxylate (880 mg, 3.8 mmol) in THF (40 mL) were added TEA (16 mL) followed by Pd(PPh₃)₂Cl₂ (184 mg, 0.26 mmol) at RT, degassed with argon for 30 minutes and CuI (496 mg, 2.6 mmol) was added to the reaction mixture. The reaction mixture was again degassed with argon for 30 minutes. The resulting reaction mixture was refluxed for 3 h. After the completion of reaction (monitored by TLC), the reaction mixture was diluted with DCM. The organic layer was washed with water and dried over anhydrous Na₂SO₄ and concentrated under reduced pressure. The crude material was purified by silica gel column chromatography (20% EtOAc/Hexane) to afford intermediate 10a (0.60 g). Mass: 478 [M⁺+1].

### Step 10b: tert-butyl 4-(3-(2-(3-hydroxyphenyl)-4-morpholinothieno[3,2-d]pyrimidin-6-yl)prop-2-ynyl)piperazine-1-carboxylate (Intermediate 10b)

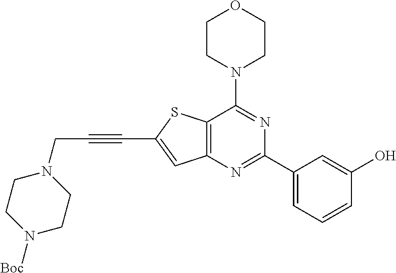

The title compound was prepared by following the procedures described in example 8, step 8c. MS m/z: 536.2 (M+H⁺).

### Step 10c: 1-(4-(3-(2-(3-hydroxyphenyl)-4-morpholinothieno[3,2-d]pyrimidin-6-yl)prop-2-ynyl)piperazin-1-yl) prop-2-en-1-one (II-a-47)

The title compound was prepared by following the procedures described in example 1, step 1e and 1f. MS m/z: 490.1 (M+H⁺).

In similar fashion, using a suitable alkyne in step 10a to couple with Intermediate 8a, the following compounds were prepared:

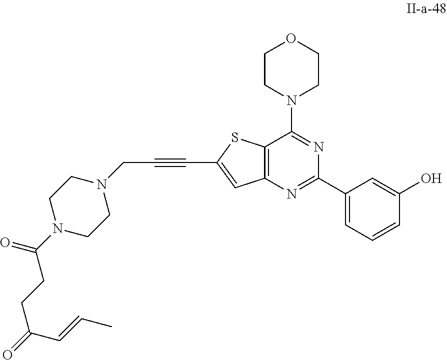

(E)-1-(4-(3-(2-(3-hydroxyphenyl)-4-morpholinothieno[3,2-d]pyrimidin-6-yl)prop-2-ynyl)piperazin-1-yl) hept-5-ene-1,4-dione (II-a-48): MS m/z: 560.2 (M+H⁺).

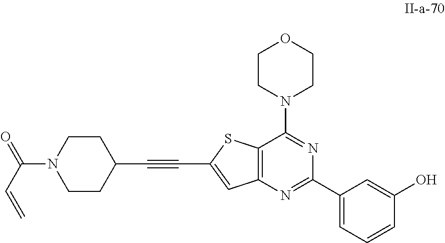

1-(4-((2-(3-hydroxyphenyl)-4-morpholinothieno[3,2-d]pyrimidin-6-yl)ethynyl)piperidin-1-yl)prop-2-en-1-one (II-a-70): Mass: 475 [M⁺+1]; TLC: 50% Ethyl acetate/hexane (R_{f}: 0.6); ¹H NMR (500 MHz, CDCl₃): δ 7.96 (d, J=7.5 Hz, 1H), 7.93 (s, 1H), 7.46 (s, 1H), 7.32 (t, J=7.5 Hz, 1H), 6.93 (dd, J=2.0 Hz, 1H), 6.63-6.55 (m, 1H), 6.29 (dd, J=1.5, 17.0 Hz, 1H), 5.70 (dd, J=2.0, 10.5 Hz, 1H), 4.10-3.77 (m, 10H), 3.03-2.96 (m, 1H), 2.0-1.95 (m, 2H), 1.85-1.65 (m, 2H).

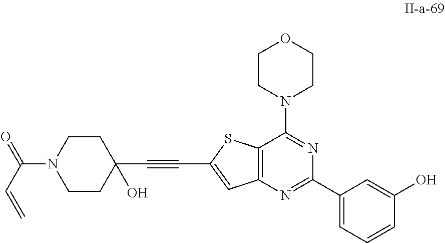

1-(4-hydroxy-4-((2-(3-hydroxyphenyl)-4-morpholinothieno[3,2-d]pyrimidin-6-yl)ethynyl)piperidin-1-yl)prop-2-en-1-one (II-a-69): TLC: 10% MeOH/DCM (Rf: 0.6); ¹H NMR (500 MHz, DMSO-d₆): δ 9.50 (s, 1H), 7.83 (t, J=8.5 Hz, 2H), 7.66 (s, 1H), 7.27 (t, J=8.5 Hz, 1H), 6.89-6.79 (m, 2H), 6.10 (dd, J=8.5 Hz, 1H), 6.04 (s, 1H), 5.67 (dd, J=8.5 Hz, 1H), 3.97 (t, J=8.5 Hz, 4H), 3.79 (t, J=8.5 Hz, 6H), 3.58-3.45 (m, 2H), 1.98-1.90 (m, 2H), 1.80-1.73 (m, 2H); Mass: 491 [M⁺+1].

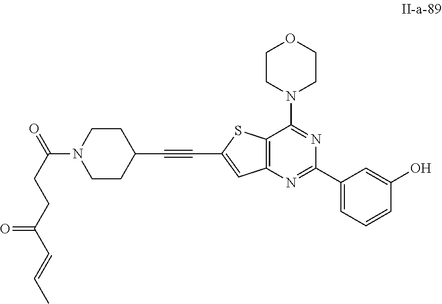

(E)-1-(4-((2-(3-hydroxyphenyl)-4-morpholinothieno[3,2-d]pyrimidin-6-yl)ethynyl)piperidin-1-yl)hept-5-ene-1,4-dione (II-a-89): MS: m/z 545.7 (ES+).

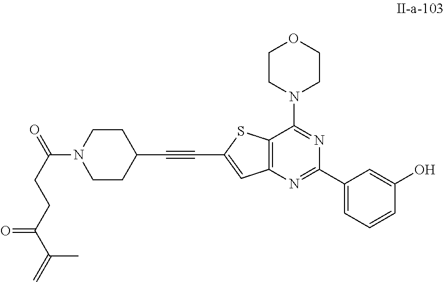

1-(4-((2-(3-hydroxyphenyl)-4-morpholinothieno[3,2-d]pyrimidin-6-yl)ethynyl)piperidin-1-yl)-5-methylhex-5-ene-1,4-dione (II-a-103): MS: m/z 545.7 (ES+).

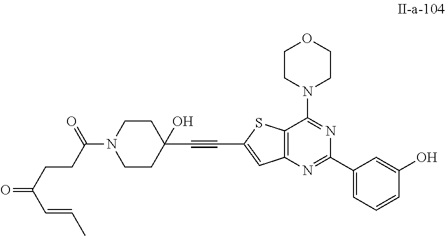

(E)-1-(4-hydroxy-4-((2-(3-hydroxyphenyl)-4-morpholinothieno[3,2-d]pyrimidin-6-yl)ethynyl)piperidin-1-yl)hept-5-ene-1,4-dione (II-a-104): MS: m/z 561.7 (ES+).

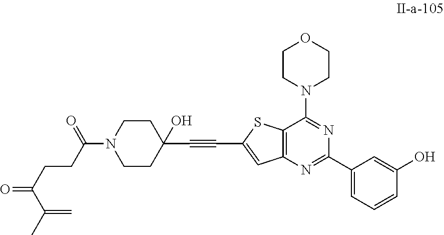

1-(4-hydroxy-4-((2-(3-hydroxyphenyl)-4-morpholinothieno[3,2-d]pyrimidin-6-yl)ethynyl)piperidin-1-yl)-5-methylhex-5-ene-1,4-dione (II-a-105): MS: m/z 561.8 (ES+).

In a similar fashion to II-a-69, using indazole-4-boronic acid in Suzuki coupling step, the following compound was prepared:

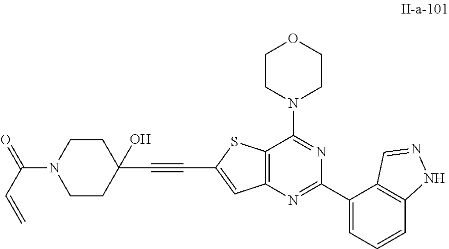

1-(4-((2-(1H-indazol-4-yl)-4-morpholinothieno[3,2-d]pyrimidin-6-yl)ethynyl)-4-hydroxypiperidin-1-yl)prop-2-en-1-one (II-a-101): MS: m/z 515.0 (ES+).

In a similar fashion, via the hydrogenation of alkyne in appropriate precursors and amide formation with appropriate carboxylic acids, the following compounds were prepared:

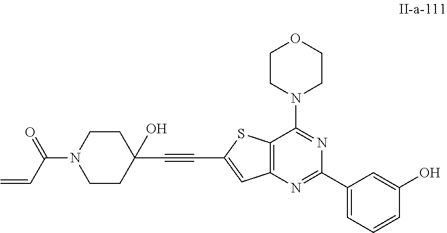

1-(4-hydroxy-4-(2-(2-(3-hydroxyphenyl)-4-morpholinothieno[3,2-d]pyrimidin-6-yl)ethyl)piperidin-1-yl)prop-2-en-1-one (II-a-111): MS: m/z 495.1 (ES+).

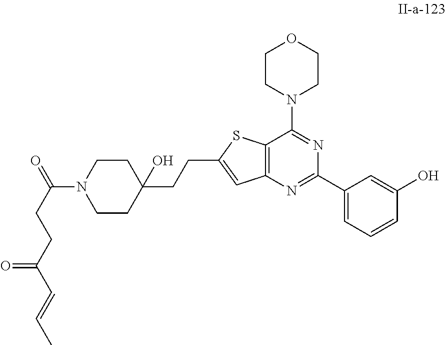

(E)-1-(4-hydroxy-4-(2-(2-(3-hydroxyphenyl)-4-morpholinothieno[3,2-d]pyrimidin-6-yl)ethyl)piperidin-1-yl)hept-5-ene-1,4-dione (II-a-123): MS: m/z 565.8 (ES+).

### Example 11

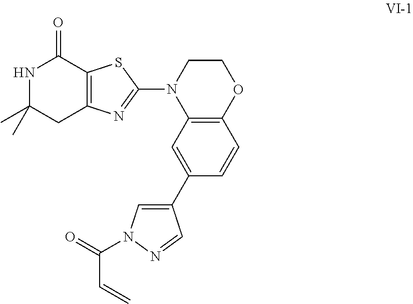

2-(6-(1-acryloyl-1H-pyrazol-4-yl)-2H-benzo[b][1,4]oxazin-4(3H)-yl)-6,6-dimethyl-6,7-dihydrothiazolo[5,4-c]pyridin-4(5H)-one (VI-1): The title compound was prepared according to the steps and intermediates as described below.

### Synthesis of Intermediate 11-I:

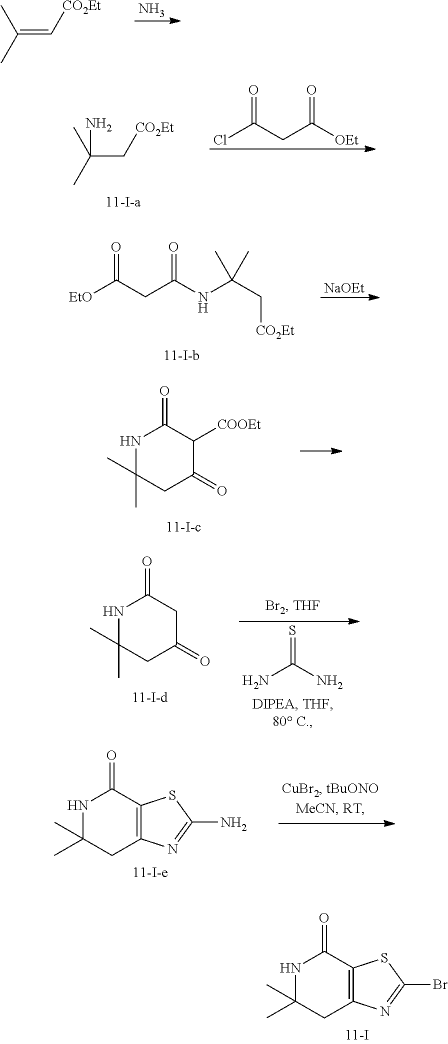

### Step II-I-a: Ethyl 3-amino-3-methylbutanoate hydrochloride salt (11-I-a)

To a solution of ethyl 3-methylbut-2-enoate (15 g, 117 mmol) in EtOH (40 mL) was added liquid ammonia (80 mL) at −70° C. and the reaction mixture stirred in a autoclave (200 Psi) at 45° C. for 16 h. After completion of the reaction (monitored by TLC), excess ammonia was removed by flashing N₂, cooled to 0° C. and HCl in dioxane (pH-2) was added. The reaction mixture was stirred for 30 minutes at 0° C., the volatiles were removed under reduced pressure and the obtained solid was washed with diethyl ether to afford 11-I-a-HCl salt (10 g, 58.8%) as white solid; TLC: 10% MeOH/DCM (R_{f}: 0.1); ¹H-NMR (DMSO d₆, 200 MHz): δ 8.33 (bs, 1H), 4.09 (q, J=7.0 Hz, 2H), 2.70 (s, 2H), 1.33 (s, 6H), 1.20 (t, J=7.0 Hz, 3H); Mass: 146 [M⁺+1].

### Step 11-I-b: Ethyl 3-(ethyl 2-carbamoylacetyl)-3-methylbutanoate (11-I-b)

To a solution of compound 11-I-a (11 g, 68.9 mmol) in DCM (150 mL) was added TEA (38.1 mL, 275 mmol) and ethyl malanoyl chloride (8.8 mL, 68.9 mmol) at 0° C. The reaction mixture was stirred at RT for 3 h. After completion of the reaction (monitored by TLC), the reaction was quenched water and extracted with DCM (2×200 mL). The combined organic layer was washed with 1N HCl (100 mL), saturated NaHCO₃ (100 mL), dried over anhydrous Na₂SO₄ and concentrated in vacuo to afford 11-I-b (11 g, 62%) as brown syrup. TLC: 30% EtOAc/Hexane (R_{f}: 0.3); ¹H-NMR (CDCl₃, 200 MHz): δ 4.28-4.07 (m, 4H), 3.24 (s, 2H), 2.74 (s, 2H), 1.45 (s, 6H), 1.35-1.20 (m, 6H); Mass: 260 [M⁺+1].

### Steps 11-I-c and 11-I-d: 6,6-Dimethylpiperidine-2,4-dione (11-I-d)

To a stirred solution of compound 11-I-b (11 g, 42.6 mmol) in toluene (120 mL) was added NaOEt (4.34 g, 63.9 mmol) in toluene (30 mL) and the reaction mixture was stirred at 80° C. for 4 h. After completion of the reaction (monitored by TLC), the reaction was quenched water, and the aqueous layer was extracted with diethyl ether (100 mL). The organic layer was separated; aqueous layer was acidified with 1N HCl and extracted with DCM (2×200 mL). The combined organic layer was dried over Na₂SO₄ and concentrated in vacuo. The obtained crude 11-I-c was dissolved in 1% H₂O/ACN (80 mL) and refluxed for 3 h. After completion of the reaction (monitored by TLC), the volatiles were removed under reduced pressure and the obtained residue was washed with diethyl ether to afford 11-I-d (3.2 g, 53.3%) as off white solid. TLC: 10% MeOH/DCM (R_{f}: 0.3); ¹H-NMR (CDCl₃+DMSO-d₆, 200 MHz): δ 7.28 (bs, NH), 3.21 (s, 2H), 2.56 (s, 2H), 1.34 (s, 6H); Mass: 142 [M⁺+1].

### Step 11-I-e: 2-Amino-6,7-dihydro-6,6-dimethylthiazolo[5,4-c]pyridin-4(5H)-one (11-I-e)

To a stirred solution of compound 11-I-d (3.2 g, 22.7 mmol) in THF (100 mL) was added Br₂ (1.13 mL, 22.7 mmol) and the reaction mixture was stirred for 10 minutes at RT followed by addition of thiourea (1.72 g, 22.7 mmol) and DIPEA (12 mL, 68.0 mmol). The reaction mixture was stirred at 80° C. for 2 h. After completion of the reaction (monitored by TLC), the reaction was quenched water and extracted with EtOAc (2×150 mL). The combined organic layer was dried over Na₂SO₄, concentrated in vacuo and the crude residue was washed with diethyl ether to afford 11-I-e (2.5 g, 56%) as yellow solid. TLC: 10% MeOH/DCM (R_{f}: 0.2); ¹H-NMR (DMSO d₆, 200 MHz): δ 7.63 (bs, 2H), 7.17 (bs, 1H), 2.61 (s, 2H), 1.22 (s, 6H); Mass: 198 [M⁺+1].

### Intermediate 11-I: 2-bromo-6,7-dihydro-6,6-dimethylthiazolo[5,4-c]pyridin-4(5H)-one

To a solution of compound 11-I-e (2.5 g, 12.7 mmol) in acetonitrile (70 mL) was added CuBr₂ (2.26 g, 10.15 mmol) and tert-butyl nitrite (1.3 g, 12.8 mmol) at RT. The reaction mixture was stirred for 2 h at RT. After completion of reaction (monitored by TLC), the reaction was quenched with 1N HCl and extracted with DCM (2×150 mL). The combined organic layer was dried over Na₂SO₄, concentrated in vacuo and the crude residue was washed with diethyl ether to afford 11-I (2 g, 60%) as brown solid; TLC: 10% MeOH/DCM (R_{f}: 0.5); ¹H-NMR (CDCl₃, 500 MHz): δ 5.48 (bs, NH), 3.02 (s, 2H), 1.4 (s, 6H); Mass: 283 [M⁺+Na].

### Synthesis of Intermediate 11-II:

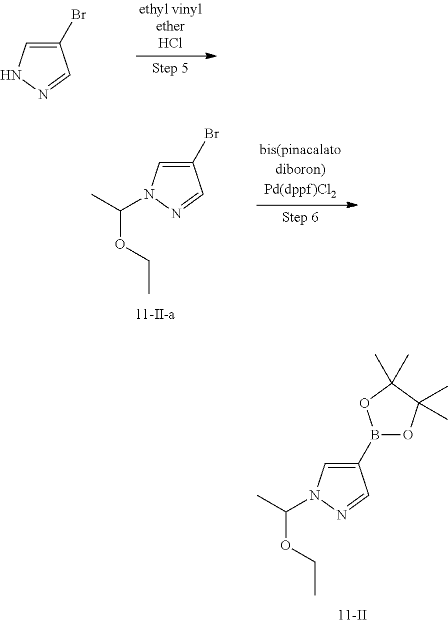

### 4-Bromo-1-(1-ethoxyethyl)-1H-pyrazole (11-II-a)

To a solution of 4-bromo-1H-pyrazole (3 g, 20.4 mmol), ethyl vinyl ether (1.76 g, 24.5 mmol) in DCM (30 mL) was added HCl (4M in dioxane, 0.16 mL), and the reaction mixture was stirred for 3 h at RT. After completion of the reaction (monitored by TLC), the reaction was neutralized with saturated NaHCO₃ solution and extracted with DCM (3×100 mL). The combined organic layers were dried over anhydrous Na₂SO₄ and concentrated in vacuo to afford 11-II-a (4.46 g, 89%) as colorless liquid; TLC: 30% EtOAc/Hexane (R_{f}: 0.7); ¹H-NMR (CDCl₃, 200 MHz): δ 7.60 (s, 1H), 7.46 (s, 1H), 5.46 (q, J=6.0 Hz, 1H), 3.55-3.25 (m, 2H), 1.63 (d, J=6.0 Hz, 3H), 1.15 (t, J=7.2 Hz, 3H); Mass: 221 [M⁺+2].

### 1-(1-ethoxyethyl)-4-(4,4,5,5-tetramethyl-1,3,2-dioxaborolan-2-yl)-1H-pyrazole (11-II)

To a solution of compound 11-II-a (600 mg, 2.73 mmol) in dioxane (15 mL) was added KOAc (800 mg, 8.2 mmol), bis(pinacolato)diboran (1.39 g, 5.4 mmol) and Pd(dppf)Cl₂ (0.06 g, 0.08 mmol) at RT. The reaction mixture was degassed by purging with argon for 30 minutes and stirred at 50° C. for 16 h. After completion of the reaction (monitored by TLC), the reaction was quenched with H₂O and extracted with EtOAc (3×100 mL). The combined organic layers were dried over anhydrous Na₂SO₄ and concentrated in vacuo. The crude compound was purified by column chromatography (15% EtOAc/Hexane) to afford 11-II (500 mg, 68.5%) as off white solid. TLC: 30% EtOAc/Hexane (R_{f}: 0.4); ¹H-NMR (CDCl₃, 200 MHz): δ 7.90 (s, 1H), 7.79 (s, 1H), 5.56 (q, J=6.0 Hz, 1H), 3.55-3.25 (m, 2H), 1.63 (d, J=6.0 Hz, 3H), 1.35 (s, 12H), 1.15 (t, J=7.2 Hz, 3H); Mass: 267 [M⁺+1].

### 2-(6-(1-acryloyl-1H-pyrazol-4-yl)-2H-benzo[b][1,4]oxazin-4(3H)-yl)-6,6-dimethyl-6,7-dihydrothiazolo[5,4-c]pyridin-4(5H)-one (VI-1)

The title compound was prepared according to the steps and intermediates as described below:

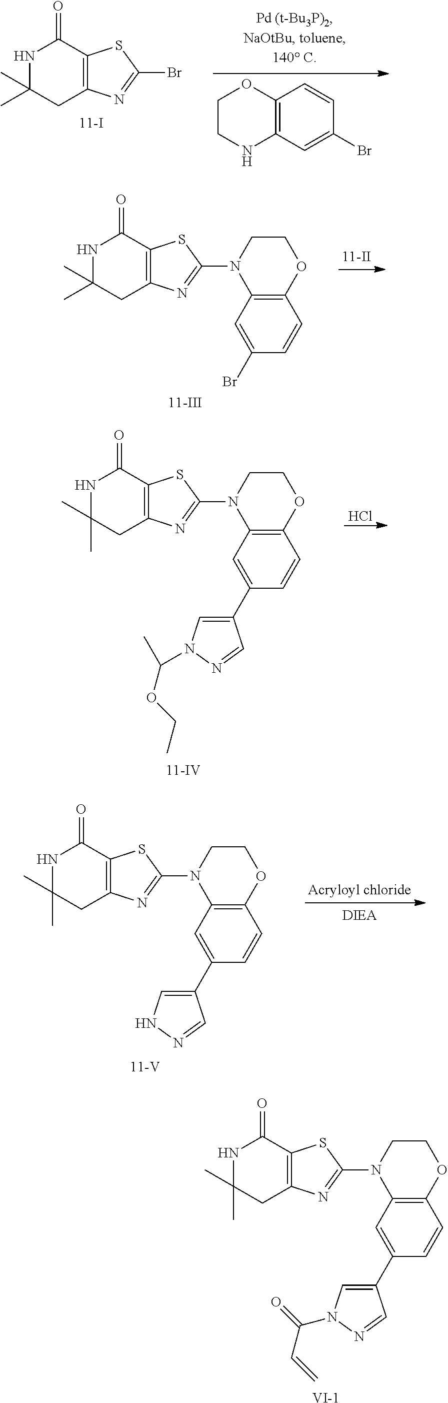

### 2-(6-bromo-2,3-dihydrobenzo[b][1,4]oxazin-4-yl)-6,7-dihydro-6,6-dimethylthiazolo[5,4-c]pyridin-4(5H)-one (11-III)

To a solution of compound 11-I (2.7 g, 10.3 mmol) in acetonitrile (100 mL) were added Cs₂CO₃ (6.71 g, 20.6 mmol), Xanthophos (476 mg, 0.82 mmol) and Pd(OAc)₂ (139 mg, 0.61 mmol) at room temperature. The reaction mixture was degassed by purging with argon and 6-bromo-3,4-dihydro-2H-benzo[b][1,4]oxazine (2.31 g, 10.3 mmol) in acetonitrile was added. The reaction mixture was degassed for 45 minutes at RT and at 85° C. for 16 h. After completion of the reaction (monitored by TLC), the reaction mixture was filtered through a pad of celite, washed with 5% MeOH/DCM and the filtrate was concentrated in vacuo. The crude compound was purified by washing with diethyl ether to afford compound 11-III (3.24 g, 80%) as brown solid. TLC: EtOAc (R_{f}: 0.4); ¹H-NMR (CDCl₃, 200 MHz): δ 8.24 (d, J=2.2 Hz, 1H), 7.14 (dd, J=2.4, 8.8 Hz, 1H), 6.83 (d, J=9.0 Hz, 1H), 5.29 (bs, NH), 4.38-4.30 (m, 2H), 4.10-4.02 (m, 2H), 2.90 (s, 2H), 1.40 (s, 6H); Mass: 394.5 [M⁺+1]; MP: 154.7° C.

### 2-(6-(1-(1-ethoxyethyl)-1H-pyrazol-4-yl)-2H-benzo[b][1,4]oxazin-4(3H)-yl)-6,6-dimethyl-6,7-dihydrothiazolo[5,4-c]pyridin-4(5H)-one (11-IV)

To a solution of compound 11-III (2.0 g, 5.0 mmol) in THF (70 mL) were added boronate ester 11-II (3.37 g, 12.7 mmol), Na₂CO₃ (1.6 g, 15.2 mmol), TBAB (653 mg, 20.3 mmol) and Pd(PPh₃)₄ (470 mg, 0.4 mmol) at room temperature. The reaction mixture was degassed by purging with argon for 45 minutes and stirred at 100° C. for 36 h. After completion of the reaction (monitored by TLC), the volatiles were removed under reduced pressure and water was added. The aqueous layer was extracted with DCM (3×100 mL), the combined organic layers was dried over anhydrous Na₂SO₄ and concentrated in vacuo. The crude compound was purified by column chromatography (3% MeOH/DCM) to afford 11-IV (850 mg, 37%) as brown solid. TLC: 5% MeOH/DCM (R_{f}: 0.4); ¹H-NMR (CDCl₃, 200 MHz): δ 8.03 (s, 1H), 7.75 (d, J=8.4 Hz, 2H), 7.20 (d, J=2.4, 8.4 Hz, 1H), 6.95 (d, J=8.4 Hz, 1H), 5.55 (q, J=6.0 Hz, 1H), 5.26 (bs, 1H), 4.40-4.30 (m, 2H), 4.25-4.15 (m, 2H), 3.55-3.35 (m, 2H), 2.90 (s, 2H), 1.73 (d, J=6.0 Hz, 3H), 1.43 (s, 6H), 1.15 (t, J=7.2 Hz, 3H); Mass: 476 [M⁺+Na] and 382 [M−71].

### 2-(6-(1H-pyrazol-4-yl)-2H-benzo[b][1,4]oxazin-4(3H)-yl)-6,6-dimethyl-6,7-dihydrothiazolo[5,4-c]pyridin-4(5H)-one (11-V)

To a solution of compound 11-IV (0.85 g, 1.87 mmol) in DCM (10 mL) was added HCl/dioxane (2 mL) at 0° C. and the reaction mixture was stirred for 2 h at RT. After completion of the reaction (monitored by TLC), the volatiles were removed under reduced pressure and the residue was washed with diisopropyl ether followed by 20% EtOAc/hexane to afford 11-V (600 mg, 84%) as off white solid. TLC: 10% MeOH/DCM (R_{f}: 0.3); ¹H-NMR (DMSO d₆, 200 MHz): δ 8.28 (d, J=8.4 Hz, 1H), 7.98 (s, 1H), 7.53 (s, 1H), 7.3 (dd, J=2.2, 8.4 Hz, 1H), 6.94 (d, J=8.4 Hz, 1H), 4.35-4.25 (m, 2H), 4.14-4.05 (m, 2H), 2.83 (s, 2H), 1.28 (s, 6H). Mass: 382 [M⁺+1].

### 2-(6-(1-acryloyl-1H-pyrazol-4-yl)-2H-benzo[b][1,4]oxazin-4(3H)-yl)-6,6-dimethyl-6,7-dihydrothiazolo[5,4-c]pyridin-4(5H)-one (VI-1)

To a stirred solution of the above compound 11-V (0.01 g, 0.024 mmol) in DCM (1.0 mL) was added TEA (0.008 g, 0.08 mmol) followed by acryloyl chloride (0.0025 g, 0.029 mmol) at RT. The reaction mixture was stirred for 0.5 h. The solvent was removed in vacuo. The crude compound was purified by prep. HPLC (25% to 90% CH₃CN aqueous containing 0.1% TFA) to give 7.0 mg of the title compound. MS m/z: 436.0 (M+1).

### Example 12

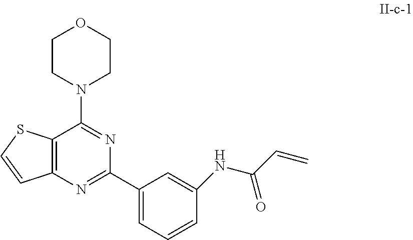

N-(3-(4-morpholinothieno[3,2-d]pyrimidin-2-yl)phenyl) acrylamide (II-c-1): The title compound was prepared according to the steps and intermediates as described below.

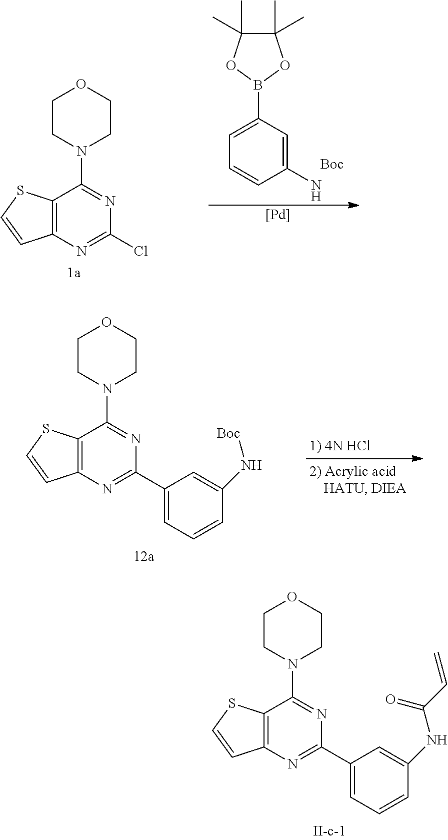

### Step 12a: tert-butyl 3-(4-morpholinothieno[3,2-d]pyrimidin-2-yl)phenylcarbamate (Intermediate 12a)

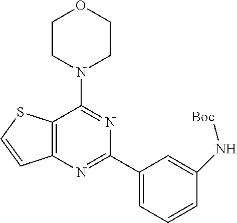

Intermediate 12a was prepared by coupling Intermediate 1a and tert-butyl 3-(4,4,5,5-tetramethyl-1,3,2-dioxaborolan-2-yl)phenylcarbamate following the procedure described in Example 4, step 4a. MS m/z: 413.3 (M+1).

### Step 12b: N-(3-(4-morpholinothieno[3,2-d]pyrimidin-2-yl)phenyl) acrylamide (II-c-1)

The title compound was prepared by following the procedures described in example 1, step 1e and 1f. MS m/z: 367.2 (M+H⁺).

### Example 13

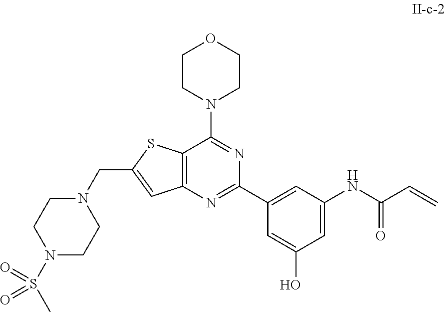

N-(3-hydroxy-5-(6-((4-(methylsulfonyl)piperazin-1-yl)methyl)-4-morpholinothieno[3,2-d]pyrimidin-2-yl)phenyl)acrylamide (II-c-2): The title compound is prepared according to the steps and intermediates as described below.

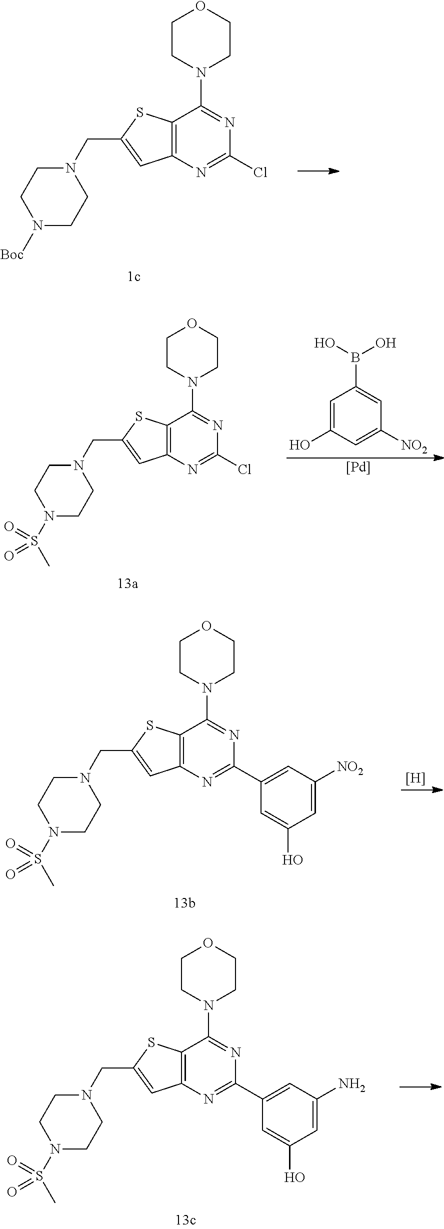

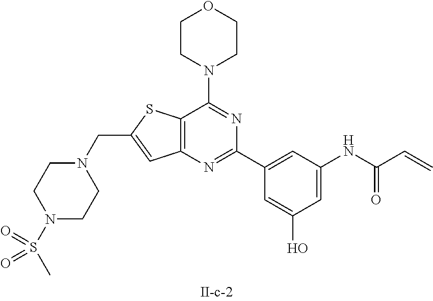

Intermediate 1c is deprotected by 4H HCl followed by the treatment with methylsulfonyl chloride to provide compound 13a. A Suzuki coupling converts compound 13a to 13b. Compound 13b is reduced to the amine 14c. 14c is then reacted with acrylic acid/HATU to produce compound II-c-2.

### Example 14

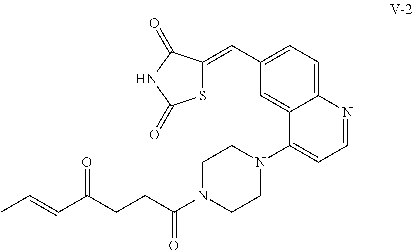

(Z)-5-((4-(4-((E)-4-oxohept-5-enoyl)piperazin-1-yl)quinolin-6-yl)methylene)thiazolidine-2,4-dione (V-2): The title compound was prepared according to the steps and intermediates as described below.

### Step 14a: Methyl 4-(4-(tert-butoxycarbonyl)piperazin-1-yl)quinoline-6-carboxylate

To methyl 4-chloroquinoline-6-carboxylate (synthesized according to WO 2007099326) (1.5 g, 6.8 mmol) in isopropanol (30 mL) was added n-Boc-piperazine (1.3 g, 7.0 mmol), and the solution was heated to 90° C. for three days. The reaction was cooled to ambient temperature, filtered and the solvent remove by rotary evaporation. The product was purified by silica chromatography (DCM/EtOAc) to give the title compound (0.51 g, 1.4 mmol). ¹H NMR (d₆DMSO) δ ppm: 8.78 (d, J=5.1 Hz, 1H), 8.66 (d, J=1.9 Hz, 1H), 8.14 (dd, J=8.7, 1.9 Hz, 1H), 8.02 (d, J=8.7 Hz, 1H), 3.91 (s, 3H), 3.64-3.58 (m, 4H), 3.20-3.14 (m, 4H), 1.43 (s, 9H); m/z 372 (M+1).

### Step 14b: Tert-butyl 4-(6-(hydroxymethyl)quinolin-4-yl)piperazine-1-carboxylate

To methyl 4-(4-(tert-butoxycarbonyl)piperazin-1-yl)quinoline-6-carboxylate (0.51 g, 1.4 mmol) in THF (10 mL) cooled to 0° C. was added lithium aluminum hydride (0.10 g, 2.7 mmol) and the reaction stirred for 30 min. The reaction was quenched by addition of excess water and the product extracted with EtOAc (3×30 mL). The combined organics were dried (MgSO₄), filtered, and the solvent removed by rotary evaporation to give the title compound as a yellow oil (0.45 g, 1.3 mmol). ¹H NMR (d₆DMSO) δ ppm: 8.64 (d, J=5.0 Hz, 1H), 7.94 (d, J=0.9 Hz, 1H), 7.89 (d, J=8.7 Hz, 1H), 7.62 (dd, J=8.3, 1.9 Hz, 1H), 6.97 (d, J=5.0 Hz, 1H), 5.38 (dd, J=6.0, 5.5 Hz, 1H), 4.67 (d, J=6.0 Hz, 1H), 3.63-3.57 (m, 4H), 3.14-3.08 (m, 4H), 1.43 (s, 9H). m/z 344 (M+1).

### Step 14c: Tert-butyl 4-(6-formylquinolin-4-yl)piperazine-1-carboxylate

To tert-butyl 4-(6-(hydroxymethyl)quinolin-4-yl)piperazine-1-carboxylate (0.45 g, 1.3 mmol) in DCM (10 mL) was added Dess-Martin periodinate (0.62 g, 1.5 mmol). The solution was stirred at ambient temperature overnight. The solution was filtered and the volatiles removed by rotary evaporation. The product was purified by silica chromatography (DCM/EtOAc) to provide the title compound as a yellow foam (0.31 g, 0.91 mmol). ¹H NMR (d₆DMSO) δ ppm: 10.20 (s, 1H), 8.80 (d, J=5.0 Hz, 1H), 8.62 (dd, J=1.4, 0.9 Hz, 1H), 8.06 (s, 1H), 8.05 (s, 1H), 7.10 (d, J=5.0 Hz, 1H), 3.67-3.62 (m, 4H), 3.24-3.21 (m, 4H), 1.44 (s, 9H). m/z 342 (M+1).

### Step 14d: (Z)-tert-butyl 4-(6-((2,4-dioxothiazolidin-5-ylidene)methyl)quinolin-4-yl)piperazine-1-carboxylate

Tert-butyl 4-(6-formylquinolin-4-yl)piperazine-1-carboxylate (0.11 g, 0.31 mmol), thiazolidine-2,4-dione (37 mg, 0.31 mmol), piperidine (25 mg, 0.31 mmol), and acetic acid (19 mg, 0.31 mmol) were combined in a microwave vial and ethanol (2 mL) added. The solution was heated at 150° C. for 30 min. in the microwave. The reaction was cooled, and the title compound collected as a yellow solid (55 mg, 0.12 mmol) by vacuum filtration, rinsing with ethanol. ¹H NMR (d₆DMSO) δ ppm: 8.74 (d, J=5.0 Hz, 1H), 8.20 (d, J=1.8 Hz, 1H), 8.04-8.01 (m, 2H), 7.89 (dd, J=8.7, 1.8 Hz, 1H), 7.06 (d, J=5.0 Hz, 1H), 3.68-3.63 (m, 4H), 3.20-3.16 (m, 4H), 1.43 (s, 9H). m/z 441 (M+1).

### Step 14e: (Z)-5-((4-(4-((E)-4-oxohept-5-enoyl)piperazin-1-yl)quinolin-6-yl)methylene)thiazolidine-2,4-dione (V-2)

(Z)-tert-butyl 4-(6-((2,4-dioxothiazolidin-5-ylidene)methyl)quinolin-4-yl)piperazine-1-carboxylate (55 mg, 0.13 mmol) was dissolved is methanol (1 mL) and 4 N HCl in dioxane (2 mL) was added. After LC-MS shows complete conversion, the volatiles were removed by rotary evaporation. The residue was taken up in DCM (3 mL) and diisopropylethylamine (0.3 mL) and split into three portions. To one portion was added (E)-4-oxohept-5-enoic acid (5.0 mg, 0.035 mmol) and HATU (15 mg, 0.039 mmol) and the solution stirred for 20 min. The solution was poured into water and washed with ethyl acetate. The water layer was concentrated on a rotary evaporator and the residue purified on by HPLC (MeCN/H₂O) to provide the title compound. ¹H NMR (d₆DMSO) δ ppm: 8.68-8.65 (m, 1H), 8.37-8.32 (m, 1H), 8.12-8.01 (m, 2H), 7.20-7.16 (m, 1H), 6.92-6.82 (m, 1H), 6.16-6.12 (m, 1H), 4.02-3.70 (m, 8H), 3.20-2.58 (m, 4H), 1.90-1.84 (m, 2H), 1.25-1.20 (m, 3H). m/z 465 (M+1).

In similar fashion, (Z)-1-(4-(6-((2-(2,6-dichlorophenylamino)-4-oxothiazol-5(4H)-ylidene)methyl)quinolin-4-yl)piperazin-1-yl)-6-methylhept-6-ene-1,5-dione (V-3) was prepared from tert-butyl 4-(6-formylquinolin-4-yl)piperazine-1-carboxylate (product of step 15c):

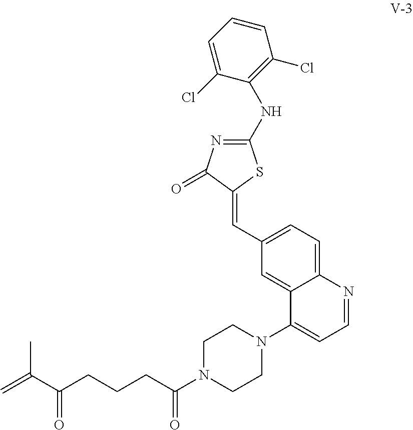

Tert-butyl 4-(6-formylquinolin-4-yl)piperazine-1-carboxylate (0.17 g, 0.50 mmol), 2-(2,6-dichlorophenylamino)thiazol-4(5H)-one (WO 2006132739) (0.13 g, 0.50 mmol), and piperidine (0.040 g, 0.50 mmol) were combined in a microwave vial and ethanol (2 mL) added. The solution was heated at 150° C. for 30 min. in the microwave. The volatiles were removed on a rotary evaporator and the residue purified by silica chromatography (EtOAc/MeOH). The purified material was dissolved in MeOH and treated with 4 N HCl in dioxane. After stirring for 1 h, the volatiles were removed by rotary evaporation. The residue was taken up in EtOAc and washed with saturated NaHCO₃ solution. The solution was dried (MgSO4), filtered and the solvent removed by rotary evaporation. The residue was taken up in DCM/diisopropylethylamine and split into three portions. To one portion was added 6-methyl-5-oxohept-6-enoic acid (23 mg, 0.15 mmol) and EDC (29 mg, 0.15 mmol). The solution was stirred overnight then purified by silica chromatography (EtOAc/MeOH) to provide the title compound. ¹H NMR (CDCl₃) δ ppm: 8.83 (d, J=5.0 Hz, 1H), 8.19 (d, J=8.7 Hz, 1H), 8.13 (d, J=1.3 Hz, 1H), 7.91 (s, 1H), 7.72 (dd, J=8.7, 1.9 Hz, 1H), 7.37 (d, J=7.8 Hz, 2H), 7.07 (dd, J=8.3, 7.7 Hz, 1H), 6.87 (d, J=5.0 Hz, 1H), 6.05 (s, 1H), 5.82 (d, J=0.9 Hz, 1H), 3.69-3.60 (m, 4H), 3.20-3.08 (m, 4H), 2.91 (dd, J=17.2, 16.1 Hz, 2H), 2.49 (dd, J=18.3, 18.3 Hz, 2H), 2.10-2.02 (m, 2H), 1.90 (s, 3H). m/z 622 (M+1).

### Example 15

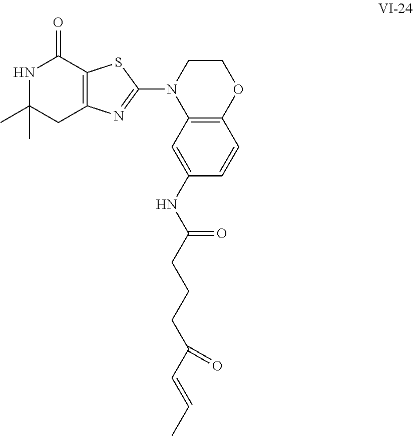

(E)-N-(4-(6,6-dimethyl-4-oxo-4,5,6,7-tetrahydrothiazolo[5,4-c]pyridin-2-yl)-3,4-dihydro-2H-benzo[b][1,4]oxazin-6-yl)-5-oxooct-6-enamide (VI-24): The title compound was prepared according to the steps and intermediates as described below.

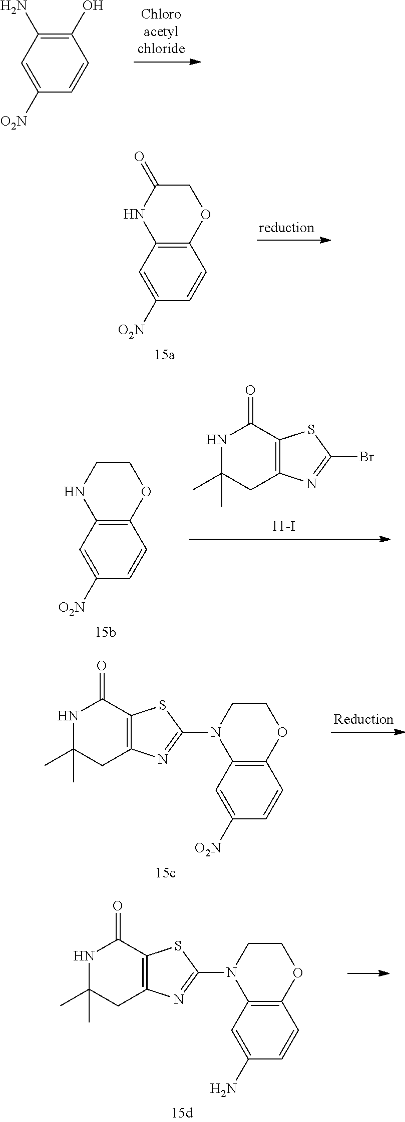

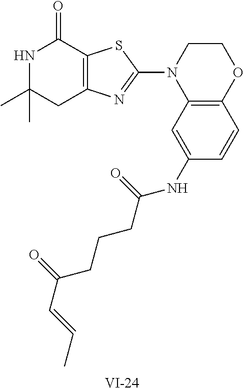

### Step 15a: 6-nitro-2H-benzo[b][1,4]oxazin-3(4H)-one (Intermediate 15a)

To a stirred solution of 2-amino-4-nitrophenol (3 g, 19.4 mmol) in DMF (25 mL) was added pyridine (1.6 mL, 19.4 mmol) and chloroacetyl chloride (1.53 mL, 19.4 mmol) at 0° C. The reaction mixture was stirred for 1 h at RT followed by addition of 60% NaH (780 mg, 19.4 mmol) and continued stirring for another 2 h at RT. After the completion of reaction (monitored by TLC), the reaction was quenched with ice cold water (150 mL), precipitated solid was filtered and dried to afford 15a (2 g, 54%) as off white solid. TLC: 60% Ethyl acetate/hexane (R_{f}: 0.4); ¹H NMR (500 MHz, CDCl₃): δ 8.05 (bs, 1H), 7.93 (d, J=9.0 Hz, 1H), 7.73 (s, 1H), 7.08 (d, J=9.0 Hz, 1H), 4.75 (s, 2H).

### Step 15b: 3,4-dihydro-6-nitro-2H-benzo[b][1,4]oxazine (Intermediate 15b)

To a stirred solution of 15a (1.7 g, 8.85 mmol) in THF (30 mL) was added BF₃ etharate (2.8 mL, 22.13 mmol) at 0° C., the reaction mixture was stirred for 1 h at RT and followed by addition of NaHB₄ (836 mg, 22.13 mmol) at 0° C. under inert atmosphere. The reaction mixture was stirred for 16 h at RT. After the completion of reaction (monitored by TLC), the reaction mixture was diluted with EtOAc/H₂O and aqueous layer was extracted with EtOAc (2×100 mL). The combined organic layer was dried over anhydrous Na₂SO₄ and concentrated in vacuo. The obtained solid was purified by ether washing to afford 15b (1 g, 63%) as off white solid. TLC: 50% Ethyl acetate/hexane (R_{f}: 0.3); ¹H NMR (500 MHz, CDCl₃): δ 7.56 (dd, J=2.5, 9.0 Hz, 1H), 7.47 (d, J=5.3 Hz, 1H), 6.8 (d, J=9.0 Hz, 1H), 4.33 (t, J=4.0 Hz, 2H), 3.48-3.44 (m, 2H); Mass: 178 [M⁺+1].

### Step 15c: 6,7-Dihydro-2-(2,3-dihydro-6-nitrobenzo[b][1,4]oxazin-4-yl)-6,6-dimethylthiazolo[5,4-c]pyridin-4(5H)-one (Intermediate 15c)

To a stirred solution of 11-I (1 g, 3.8 mmol) in acetonitrile (25 mL) was added compound 15b (680 mg, 3.8 mmol), Xanthophos (176 mg, 0.3 mmol), Pd(OAc)₂ (52 mg, 0.2 mmol) and Cs₂CO₃ (2.5 g, 7.6 mmol) at RT. The reaction mixture was degassed with argon for 45 minutes and stirred for 6 h at 80° C. After the completion of reaction (monitored by TLC), the volatiles were removed in vacuo, diluted with water and extracted with DCM (2×100 mL). The combined organic layer was dried over anhydrous Na₂SO₄ and concentrated in vacuo. The crude residue was washed with diethyl ether to afford 15c (1 g, 73%) as light brown solid. TLC: Ethyl acetate (R_{f}: 0.3); ¹H NMR (200 MHz, CDCl₃): δ 9.32 (d, J=2.6 Hz, 1H), 7.94 (dd, J=2.6, 9.0 Hz, 1H), 7.04 (d, J=9.0 Hz, 1H), 5.33 (bs, 1H), 4.46 (t, J=4.4 Hz, 2H), 4.07 (t, J=4.6 Hz, 2H), 2.95 (s, 2H) and 1.41 (s, 6H).

### Step 15d: 2-(6-amino-2,3-dihydrobenzo[b][1,4]oxazin-4-yl)-6,7-dihydro-6,6-dimethylthiazolo[5,4-c]pyridin-4(5H)-one (Intermediate 15d)

To a stirred solution of 15c (1 g, 2.7 mmol) in EtOAc/MeOH (1:1, 40 mL) was added Pd/C (100 mg). The reaction mixture was stirred under hydrogen atmosphere (60 Psi) for 36 h at RT. After the completion of reaction (monitored by TLC), the reaction mixture was filtered through a pad of celite and filtrate was concentrated in vacuo. The crude residue was recrystallised from DCM/hexane to afford 15d (520 mg, 57%) as off white solid. TLC: 10% MeOH/DCM (R_{f}: 0.4); ¹H NMR (500 MHz, CDCl₃): δ 7.34 (d, J=3.0 Hz, 1H), 6.76 (d, J=8.5 Hz, 1H), 6.42 (dd, J=2.5, 8.0 Hz, 1H), 5.17 (bs, 2H), 4.25 (t, J=4.0 Hz, 2H), 4.11 (t, J=5.5 Hz, 2H), 3.5 (bs, 2H), 2.87 (s, 2H), 1.39 (s, 6H); Mass: 331 [M⁺+1]; MP: 244.8° C.

### Step 15e: (E)-N-(4-(6,6-dimethyl-4-oxo-4,5,6,7-tetrahydrothiazolo[5,4-c]pyridin-2-yl)-3,4-dihydro-2H-benzo[b][1,4]oxazin-6-yl)-5-oxooct-6-enamide (VI-24)

The title compound was prepared from Intermediate 15d and (E)-5-oxooct-6-enoic acid according to the HATU procedure described in Example 1, step 1f. MS m/z: 469.1 (M+H⁺); ¹H NMR (400 MHz, DMSO-d6): δ: 9.89 (1H m), 8.34 (1H d), 7.54 (1H s), 7.25 (1H, dd), 6.87 (2H m), 6.115 (1H dq), 4.25 (2H, bt), 4.11 (2H, bt), 2.8 (2H, s), 2.6 (2H, t), 2.3 (2H, t), 1.85 (3H, dd), 1.8 (2H, m), 1.28 (6H, s).

The following compound was prepared by starting with Intermediate 15d and following the procedures or procedure combinations described in previous examples.

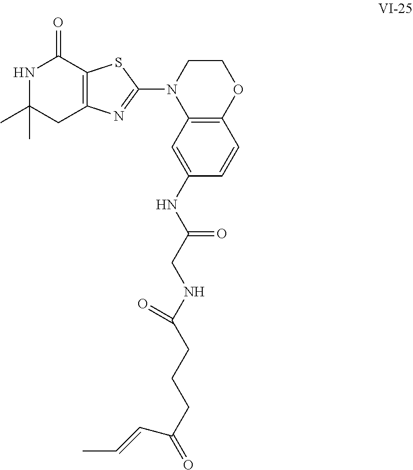

MS m/z: 524.2 (ES−).

### Example 16

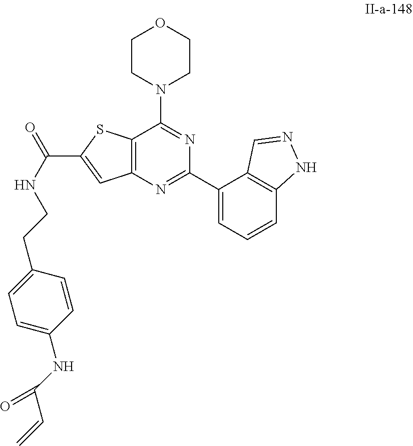

N-(4-acrylamidophenethyl)-2-(1H-indazol-4-yl)-4-morpholinothieno[3,2-d]pyrimidine-6-carboxamide (II-a-148): The title compound was prepared according to the steps and intermediates as described below.

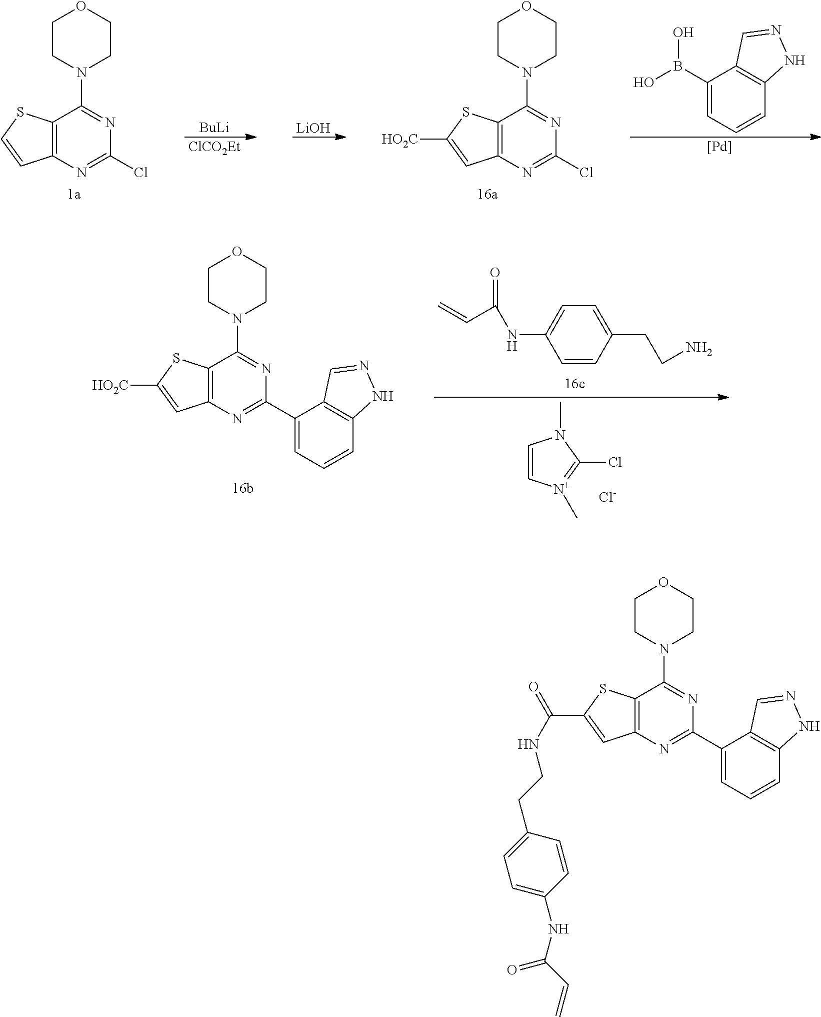

### Step 16a: 2-chloro-4-morpholinothieno[3,2-d]pyrimidine-6-carboxylic acid (Intermediate 16a)

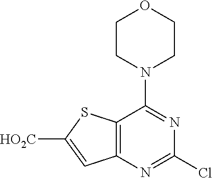

Under Argon, to a stirring solution of Intermediate 1a (2.0 g, 7.8 mmol) in 40 mL of anhydrous tetrahedron furan at −78° C., was added dropwise of n-BuLi (5 mL of 2.5 N in heptanes, 12.5 mmol). After stirring at −78° C. for additional 1 hr, ethyl chloroformate (15.6 mmol) was added slowly. The resulting mixture was warmed up to rt slowly, and stirred 2 hr at rt. The reaction was then quenched with 1N HCl, and the crude product was extracted with ethyl acetate, washed with water, brine, and dried over anhydrous sodium sulfate. After filtration and concentration, the residue was subject to basic hydrolysis using LiOH (900 mg, 37.5 mmol) in 25 mL of THF and 25 mL of water at rt for 4 hr. The reaction was acidified with 1N HCl, and 1.5 g of off-white solid was collected as desired product. LC-MS: m/z 299.9 (ES+)

### Step 16b: 2-(1H-indazol-4-yl)-4-morpholinothieno[3,2-d]pyrimidine-6-carboxylic acid (Intermediate 16b)

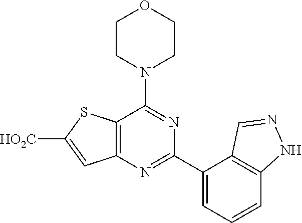

A mixture of Intermediate 16a (90 mg, 0.3 mmol), 1H-indazol-4-ylboronic acid (64 mg, 0.39 mmol), 17 mg of Pd(PPh₃)₄ in 1 mL of DMA and 0.5 mL of 1M aqueous Na₂CO₃, was heated at 120° C. for 30 min under microwave condition. The reaction mixture was diluted with 2 mL of MeOH and 1 mL of water, and filtrated. 1N of aqueous HCl and 4 mL of acetonitrile were added into the filtrate, the browny solid was then filtered and dried, giving desired acid 91 mg (80%). LC-MS: m/z 382.1 (ES+).

### Intermediate 16c: N-(4-(2-aminoethyl)phenyl)acrylamide Trifluoroacetic Acid Salt

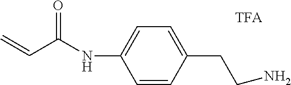

At −10° C., to a stirring solution of tert-butyl 4-aminophenethylcarbamate (3.54 g, mmol) and 3 mL of DIPEA in 100 mL of dichloromethane, was added acryloyl chloride (1.35 mL, 16.5 mmol). After 10 min, the reaction was quenched by added 5 mL of 1 N aqueous HCl. The reaction mixture was concentrated on a rotavapor, and 100 mL of ethyl acetate was added. The mixture was washed with dilute HCl, water, brine and dried over anhydrous sodium sulfate. After filtration and concentration, the residue was re-dissolved in mL of dichloromethane, 10 mL of trifluoroacetic acid was added slowly. The reaction mixture was stirred at rt for 2 hr, and was concentrated to minimum volume on rotavapor. Ethyl ether was added in slowly, the solid was filtrated, giving desired TFA salt in almost quantitative yield. MS: m/z 191.1 (ES+).

### N-(4-acrylamidophenethyl)-2-(1H-indazol-4-yl)-4-morpholinothieno[3,2-d]pyrimidine-6-carboxamide (II-a-148)

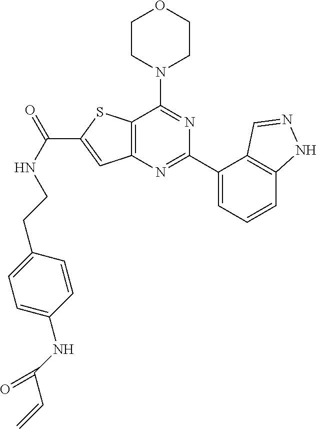

To a stirring solution of Intermediate 16b (175 mg, 0.46 mmol), Intermediate 16c (140 mg, 0.46 mmol), 400 uL of DIPEA in 2 mL of DMA and 4 mL of dichloromethane, was added 2-chloro-1,3-dimethylimidazolidinium chloride (100 mg, 0.60 mmol) in 1 mL of dichloromethane. After 5 min, the reaction mixture was poured into 50 mL of 1% NaHCO₃ aqueous solution. The solid was collected and redissolved into 20 mL of DCM-MeOH (v/v 3/1). After removing the insoluble materials, the solution was concentrated giving 129 mg of pale-yellow solid. MS: m/z 554.1 (ES+).

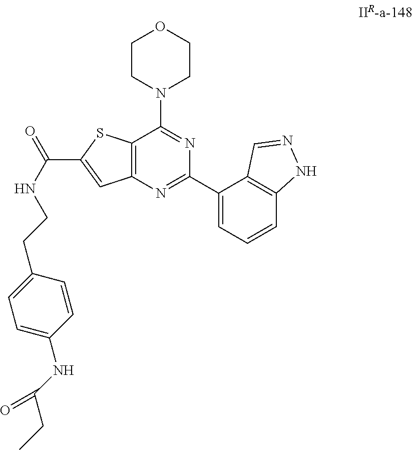

2-(1H-indazol-4-yl)-4-morpholino-N-(4-propionamidophenethyl)thieno[3,2-d]pyrimidine-6-carboxamide (II^{R}-a-148): This compound was made by hydrogenation of II-a-148 in the presence of 5% palladium/C. MS: m/z 556.1 (ES+).

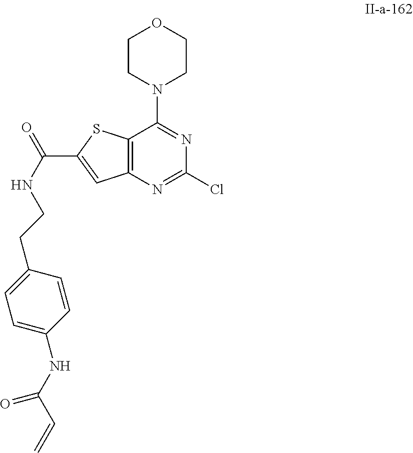

N-(4-acrylamidophenethyl)-2-chloro-4-morpholinothieno[3,2-d]pyrimidine-6-carboxamide (II-a-162): This compound was prepared by directly reacting Intermediate 16b with Intermediate 16c. MS: m/z 472.1 (ES+).

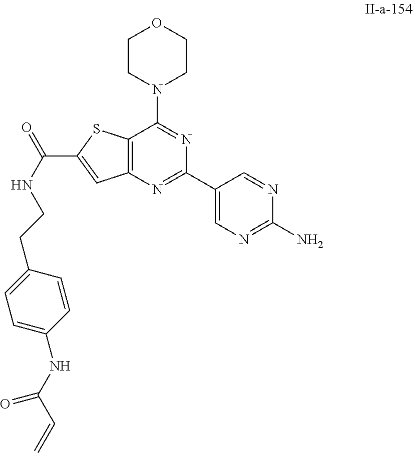

N-(4-acrylamidophenethyl)-2-(2-aminopyrimidin-5-yl)-4-morpholinothieno[3,2-d]pyrimidine-6-carboxamide (II-a-154). In a similar way to making II-a-148, the title compound was prepared using 2-aminopyrimidine-5-boronic acid in step 16b. MS: m/z 531.0 (ES+).

In a similar fashion, using an appropriate amine counterpart in place of Intermediate 16c, the following compounds were synthesized:

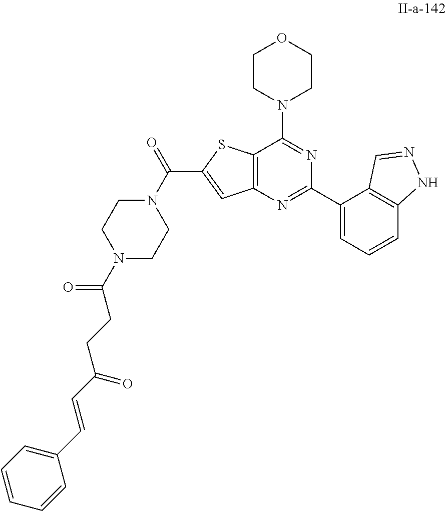

(E)-1-(4-(2-(1H-indazol-4-yl)-4-morpholinothieno[3,2-d]pyrimidine-6-carbonyl)piperazin-1-yl)-6-phenylhex-5-ene-1,4-dione (II-a-142): MS: m/z 636.2 (ES+).

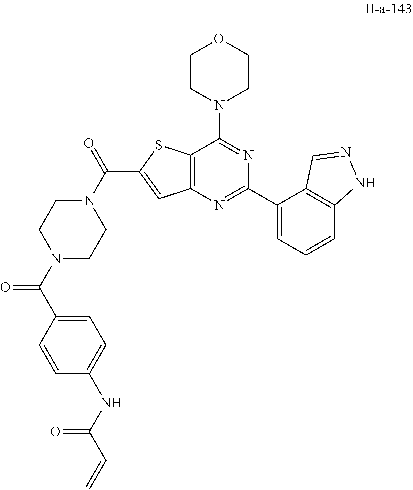

N-(4-(4-(2-(1H-indazol-4-yl)-4-morpholinothieno[3,2-d]pyrimidine-6-carbonyl)piperazine-1-carbonyl)phenyl)acrylamide (II-a-143). MS: m/z 623.3 (ES+).

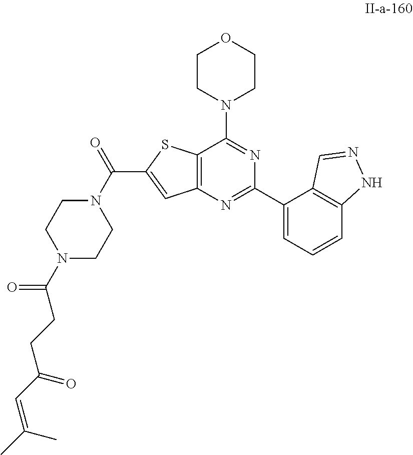

1-(4-(2-(1H-indazol-4-yl)-4-morpholinothieno[3,2-d]pyrimidine-6-carbonyl)piperazin-1-yl)-6-methylhept-5-ene-1,4-dione (II-a-160). MS: m/z 588.2 (ES+).

In a similar fashion, using 3-hydroxyphenylboronic acid in step 16b and an appropriate amine in step 16c, the following compounds were synthesized:

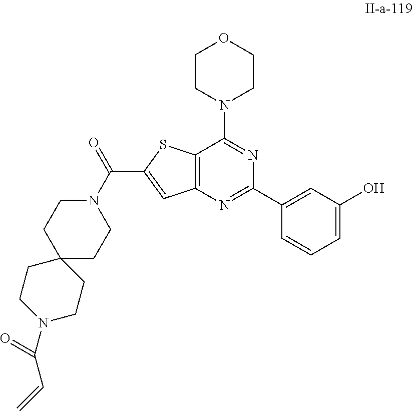

1-(9-(2-(3-hydroxyphenyl)-4-morpholinothieno[3,2-d]pyrimidine-6-carbonyl)-3,9-diazaspiro[5.5]undecan-3-yl)prop-2-en-1-one (II-a-119). MS: m/z 548.3 (ES+).

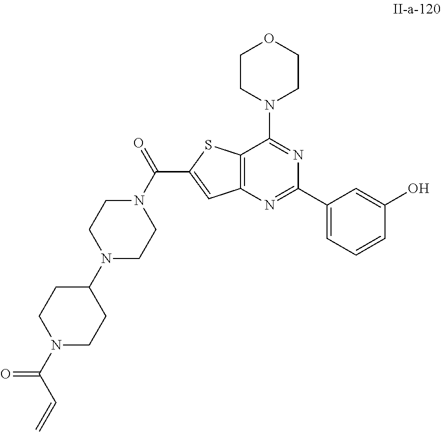

1-(4-(4-(2-(3-hydroxyphenyl)-4-morpholinothieno[3,2-d]pyrimidine-6-carbonyl)piperazin-1-yl)piperidin-1-yl)prop-2-en-1-one (II-a-120). MS: m/z 617.3 (ES+).

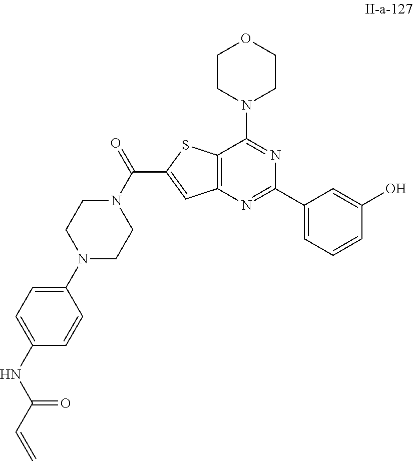

N-(4-(4-(2-(3-hydroxyphenyl)-4-morpholinothieno[3,2-d]pyrimidine-6-carbonyl)piperazin-1-yl)phenyl)acrylamide (II-a-127). MS: m/z 571.3 (ES+).

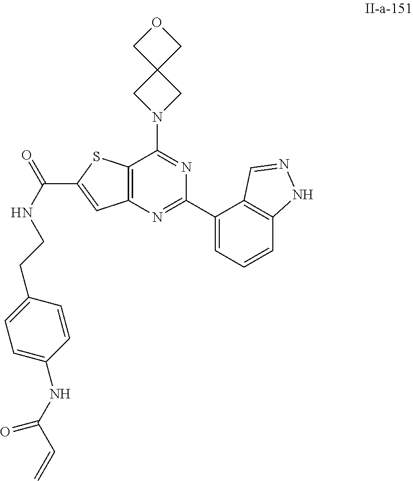

N-(4-acrylamidophenethyl)-2-(1H-indazol-4-yl)-4-(2-oxa-6-azaspiro[3.3]heptan-6-yl)thieno[3,2-d]pyrimidine-6-carboxamide (II-a-151): The title compound was prepared in a similar fashion as described for II-a-148 by using 2-oxa-6-azaspiro[3.3]heptane instead of morpholine at the very beginning MS: m/z 566.2 (ES+).

### Example 17

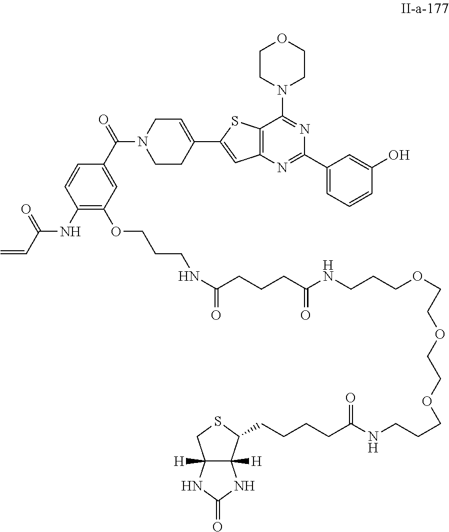

N1-(3-(2-acrylamido-5-(4-(2-(3-hydroxyphenyl)-4-morpholinothieno[3,2-d]pyrimidin-6-yl)-1,2,3,6-tetrahydropyridine-1-carbonyl)phenoxy)propyl)-N5-(15-oxo-19-((3aR,4R,6aS)-2-oxohexahydro-1H-thieno[3,4-d]imidazol-4-yl)-4,7,10-trioxa-14-azanonadecyl)glutaramide (II-a-177): The title compounds was prepared according to the steps and intermediates as described below.

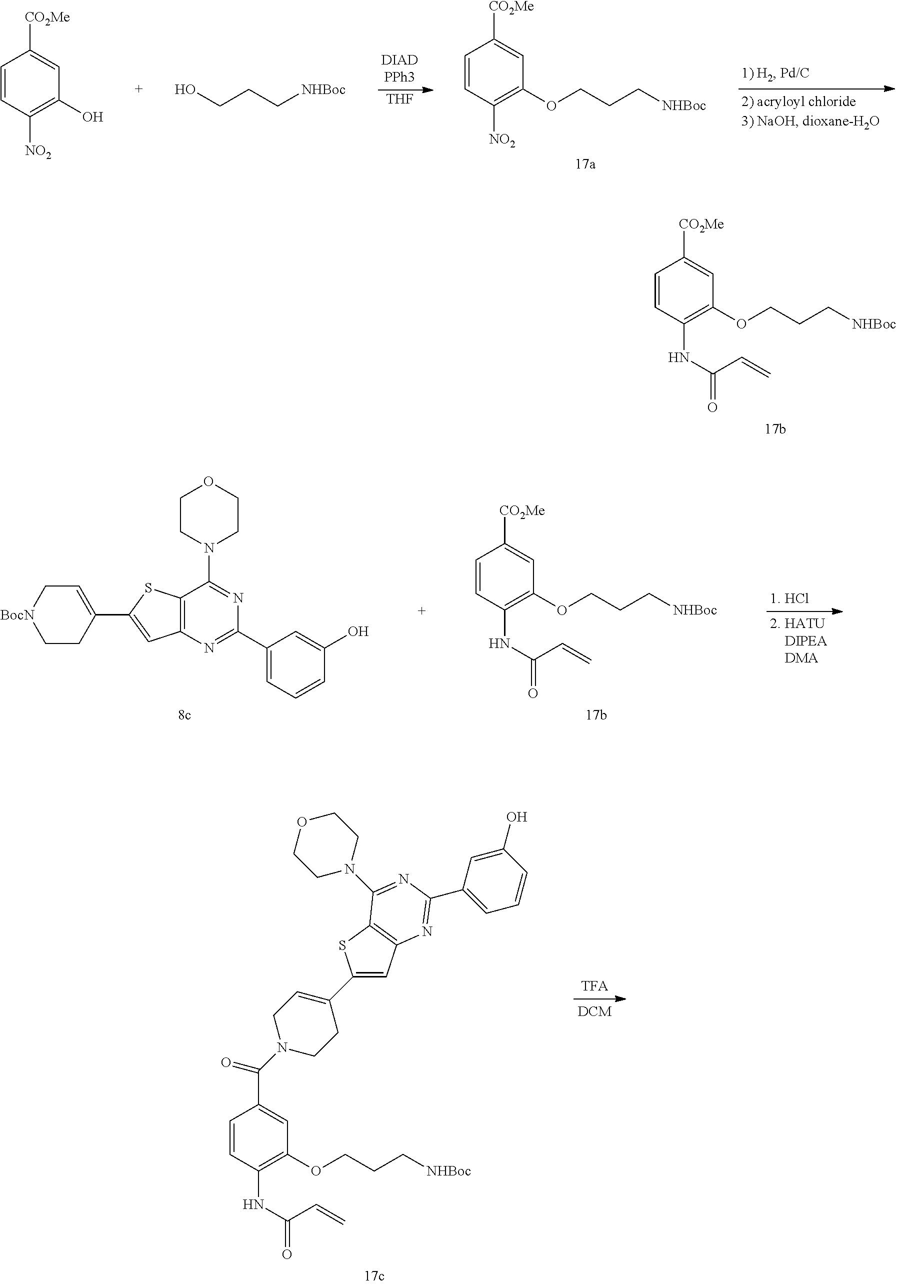

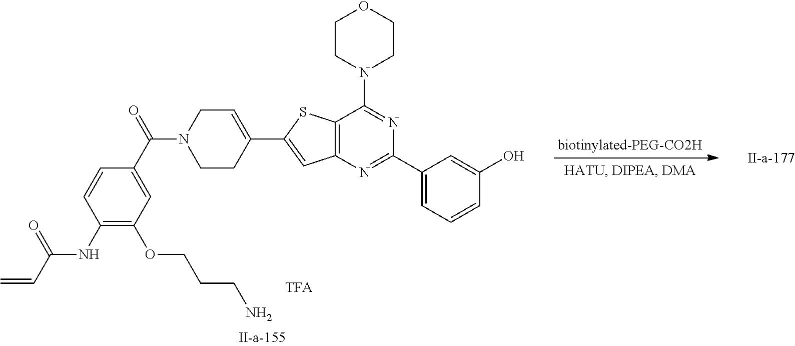

### Step 17a: Methyl 3-(3-(tert-butoxycarbonylamino)propoxy)-4-nitrobenzoate (Intermediate 17a)

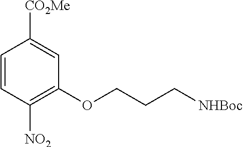

Under Nitrogen, to a mixture of methyl 3-hydroxy-4-nitrobenzoate (400 mg, 2.0 mmol), tert-butyl 3-hydroxypropylcarbamate (350 mg, 2.0 mmol), triphenylphosphine (530 mg, 2.0 mmol) in 6 mL of anhydrous tetrahydrofuran, was added diisopropyl azodicarboxylate (0.4 mL). The resulting mixture was stirred at room temperature for 1 hr. After concentration, the residue was purified by column chromatography with heptanes/ethyl acetate (v/v 2/1), giving about 1.0 g of yellowish oil. MS: m/z 255.2 (M-Boc, ES+). The product was used directly in following step.

### Step 17b: 4-acrylamido-3-(3-(tert-butoxycarbonylamino)propoxy)benzoic acid (Intermediate 17b)

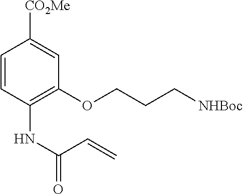

Crude Intermediate 17a obtained above was stirred overnight under hydrogen with 100 mg of 10% Pd/C in 20 mL of MeOH. The reaction mixture was filtered and concentrated to give foamy solid as desired anline (MS: m/z 225.2 M-Boc, ES+).

To a solution of the aniline obtained above (140 mg) in 4 mL of dichloromethane with 200 uL of DIPEA at −20° C., was added acryloyl chloride (40 uL). After 15 min, the reaction mixture was subjected to aqueous workup, and purified by column chromatography on silica gel with heptanes/ethyl acetate (v/v 3/1), giving 120 mg white solid. (MS: 279.0 M-Boc, ES+).

The acrylamide obtained above (38 mg, 0.1 mol) was stirred with 0.4 mL of dioxane and 0.4 mL of 1N NaOH at room temperature overnight. The desired acid (18 mg) was filtered out after the neutralization with 1N HCl. MS: m/z 265.1 (M-Boc, ES+).

### Step 17c: tert-butyl 3-(2-acrylamido-5-(4-(2-(3-hydroxyphenyl)-4-morpholinothieno[3,2-d]pyrimidin-6-yl)-1,2,3,6-tetrahydropyridine-1-carbonyl)phenoxy)propylcarbamate (Intermediate 17c)

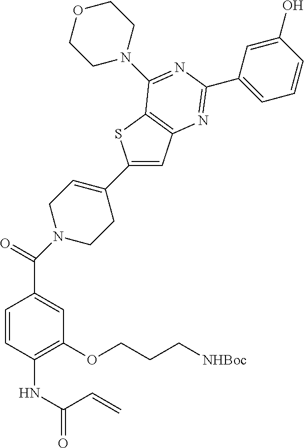

Intermediate 8c (34 mg, 67 umol) in 1 mL of dichloromethane was treated with 1 mL of 4.0 N HCl in dioxane for 1 hr. After 1 hr, the solvent was removed under reduced pressure. The residue was re-dissolved in 1 mL of DMA, 23 mg of Intermediate 17b (63 umol), and 200 uL of DIPEA were then added, followed by 26 mg of HATU (68 umol). The reaction mixture was extracted with 30 mL of EtOAc, washed with water, brine, and dried over Na₂SO₄. After filtration and concentration, the residue was purified by column chromatography on silica gel with 5% MeOH in dichloromethane, giving 27 mg of desired Intermediate 17c. MS: m/z 741.2 (ES+).

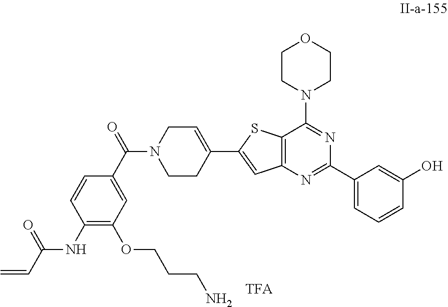

N-(2-(3-aminopropoxy)-4-(4-(2-(3-hydroxyphenyl)-4-morpholinothieno[3,2-d]pyrimidin-6-yl)-1,2,3,6-tetrahydropyridine-1-carbonyl)phenyl)acrylamide (II-a-155). The title compound was made by removing the Boc-group of Intermediate 17c with TFA in dichloromethane. MS: m/z 641.2 (ES+).

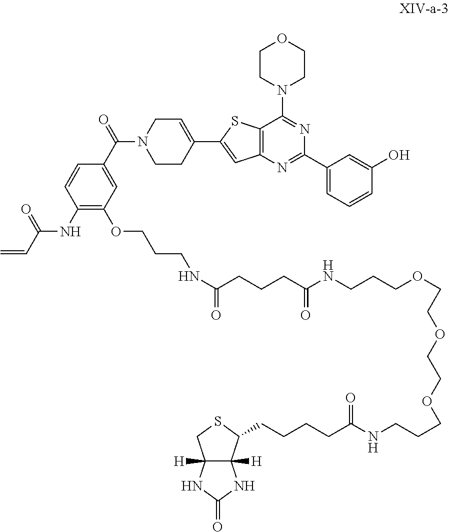

N1-(3-(2-acrylamido-5-(4-(2-(3-hydroxyphenyl)-4-morpholinothieno[3,2-d]pyrimidin-6-yl)-1,2,3,6-tetrahydropyridine-1-carbonyl)phenoxy)propyl)-N5-(15-oxo-19-((3aR,4R,6aS)-2-oxohexahydro-1H-thieno[3,4-d]imidazol-4-yl)-4,7,10-trioxa-14-azanonadecyl)glutaramide (XIV-a-3): The title compound was made by 8.8 mg of II-a-155, 8.0 mg of biotinylated acid in the presence of 200 uL of DIPEA, 8 mg of HATU in 0.5 mL of DMA. The final product was purified by prep-HPLC. MS: m/z 1183.3 (ES+).

### Example 18

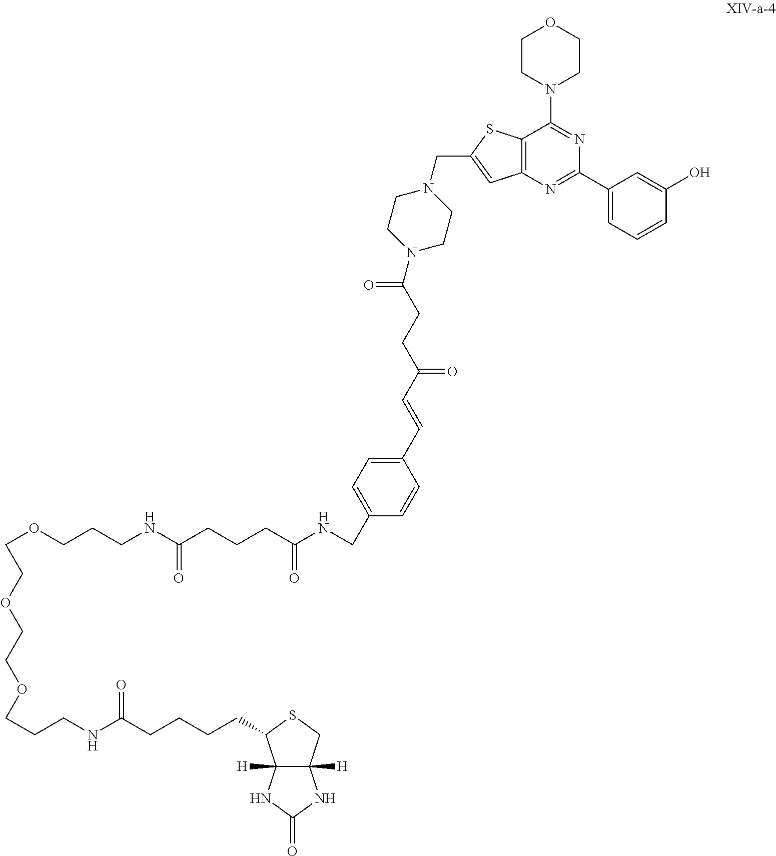

N¹-(4-((E)-6-(4-((2-(3-hydroxyphenyl)-4-morpholinothieno[3,2-d]pyrimidin-6-yl)methyl)piperazin-1-yl)-3,6-dioxohex-1-enyl)benzyl)-N⁵-(15-oxo-19-((3aS,4S,6aR)-2-oxohexahydro-1H-thieno[3,4-d]imidazol-4-yl)-4,7,10-trioxa-14-azanonadecyl)glutaramide (XIV-a-4). The title compound was prepared through the following intermediate as described.

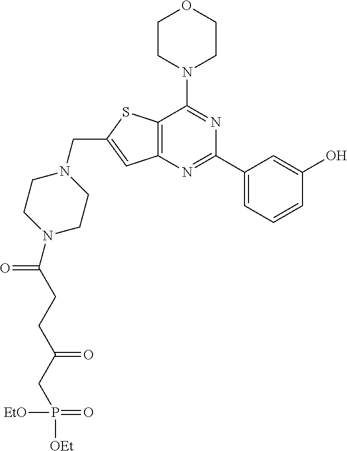

Diethyl 5-(4-((2-(3-hydroxyphenyl)-4-morpholinothieno[3,2-d]pyrimidin-6-yl)methyl)piperazin-1-yl)-2,5-dioxopentylphosphonate: The title phosphonate intermediate was prepared in a similar fashion as described for making Intermediate 9b, using 3-hydroxyphenylboronic acid in place of 4-indazoleboronic acid. MS: m/z 646.3 (ES+).

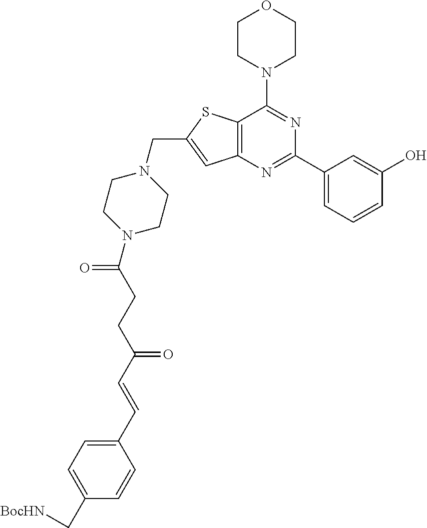

(E)-tert-butyl 4-(6-(4-((2-(3-hydroxyphenyl)-4-morpholinothieno[3,2-d]pyrimidin-6-yl)methyl)piperazin-1-yl)-3,6-dioxohex-1-enyl)benzylcarbamate: A mixture of the phosphonate above (13 mg, 20 umol), tert-butyl 4-formylbenzylcarbamate (10 mg, 40 umol), potassium carbonate (40 mg) in 1 mL of DMA and 100 uL of water was heated at 70° C. for 4 hrs. After filtration, the reaction mixture was purified by prep-HPLC, giving 10 mg of desired enone as white solid. MS: m/z 727.3 (ES+).

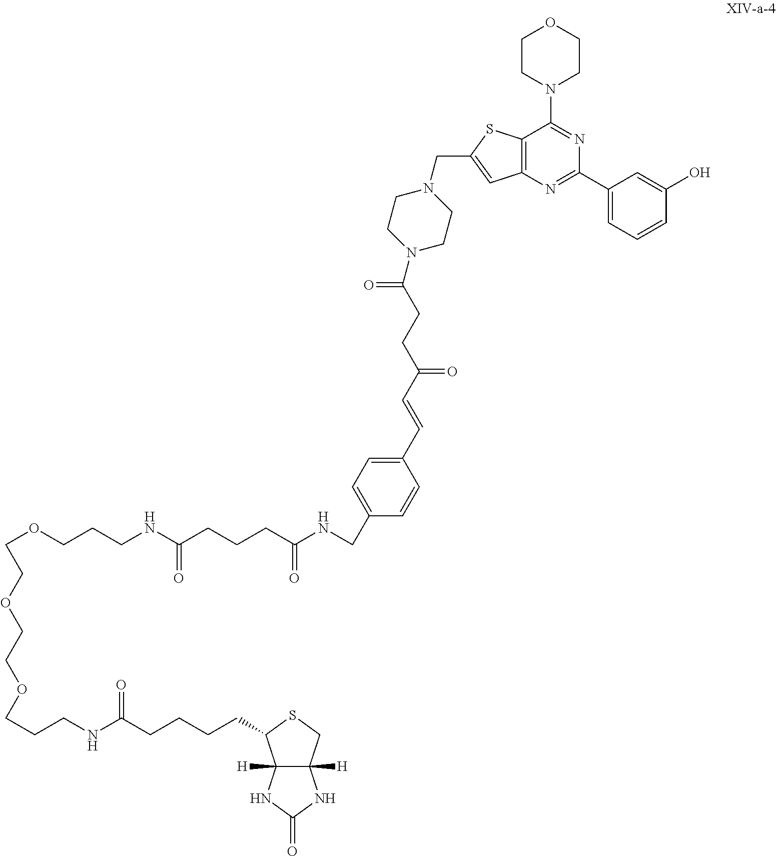

N¹-(4-((E)-6-(4-((2-(3-hydroxyphenyl)-4-morpholinothieno[3,2-d]pyrimidin-6-yl)methyl)piperazin-1-yl)-3,6-dioxohex-1-enyl)benzyl)-N⁵-(15-oxo-19-((3aS,4S,6aR)-2-oxohexahydro-1H-thieno[3,4-d]imidazol-4-yl)-4,7,10-trioxa-14-azanonadecyl)glutaramide (II-a-178). The enone intermediate (7.5 mg, ˜10 umol) was treated with 1 mL of TFA in 1 mL of dichloromethane at room temperature for 30 min. The solvent was removed, and the residue was dissolved in 1 mL of DMA, followed by addition of 100 uL of DIPEA, 9 mg of biotinylated acid, and 9 mg of HATU. The reaction mixture was stirred for 30 min, then subject to prep-HPLC purification, giving 6 mg of desired compounds. MS: m/z 1169.4 (ES+).

### Example 19

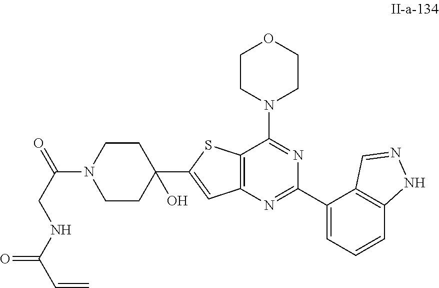

N-(2-(4-(2-(1H-indazol-4-yl)-4-morpholinothieno[3,2-d]pyrimidin-6-yl)-4-hydroxypiperidin-1-yl)-2-oxoethyl)acrylamide (II-a-134). The title compound was prepared according to the steps and intermediates as described below.

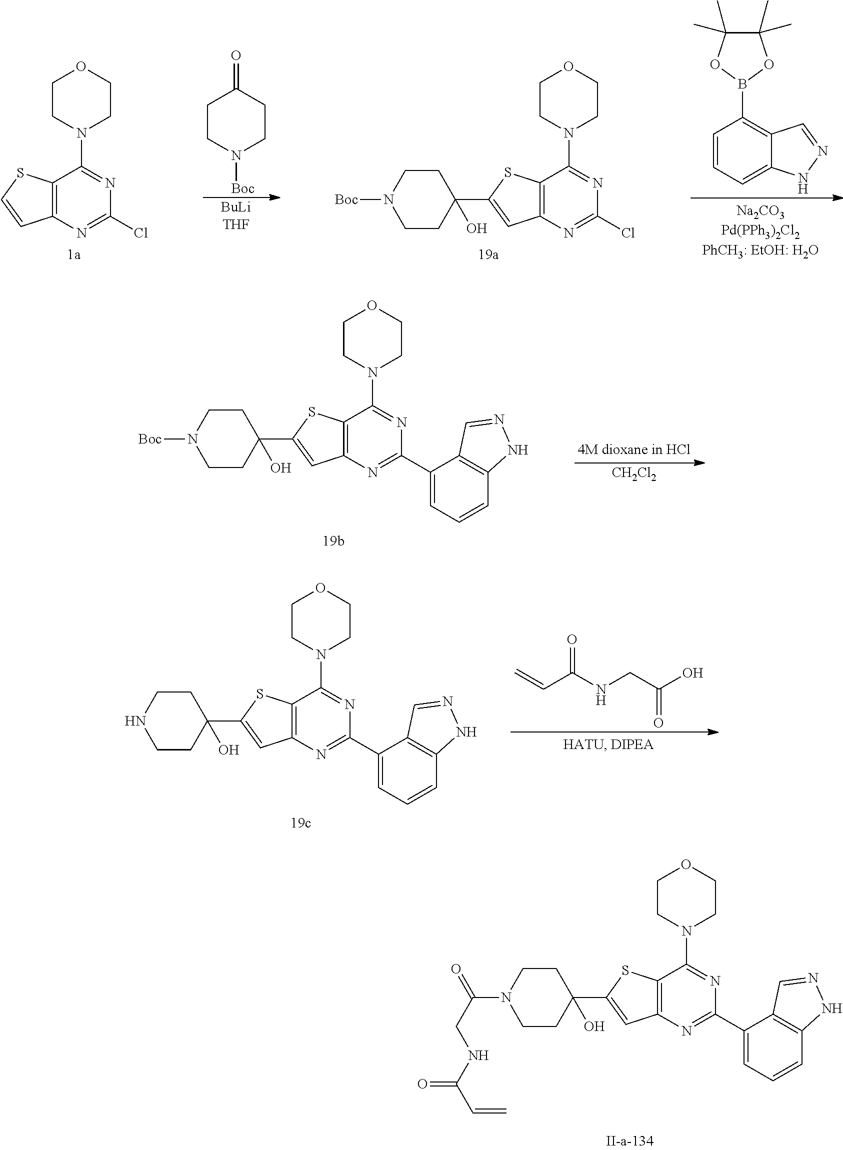

### Step 19a: tert-Butyl 4-(2-chloro-4-morpholinothieno[3,2-d]pyrimidin-6-yl)-4-hydroxy piperidine-1-carboxylate (Intermediate 19a)

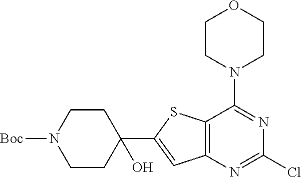

To a stirred solution of Intermediate 1a (2.0 g, 7.84 mmol) in THF (50 mL) at −78° C. was added n-BuLi (1.0 g, 15.62 mmol) and allowed to stir at −10° C. for 1 h. A solution of tert-butyl 4-oxopiperidine-1-carboxylate (4.6 g, 23.52 mmol) in THF (50 mL) was added to the reaction mixture at −78° C. and stirring was continued for another 3 h. After the completion of the staring material (by TLC), the reaction mixture was quenched with water (20 mL) and extracted with EtOAc (3×75 mL). The combined organic extracts were washed with water (100 mL), brine (20 mL), dried over anhydrous Na₂SO₄ and concentrated under reduced pressure. The obtained crude compound was purified by column chromatography eluting with 50% EtOAc/Hexane to afford Intermediate 19a (2 g, 57%). TLC: 50% EtOAc/Hexane (Rf: 0.3)

### Step 19b: tert-Butyl 4-(2-(1H-indazol-4-yl)-4-morpholinothieno[3,2-d]pyrimidin-6-yl)-4-hydroxypiperidine-1-carboxylate (Intermediate 19b)

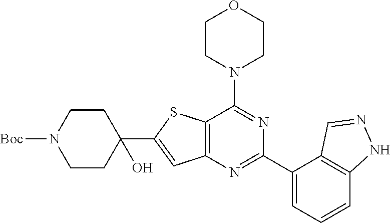

To a stirred mixture of Intermediate 19a (0.5 g, 1.09 mmol), indazole-4-boronic ester (0.53 g, 2.18 mmol) and Na₂CO₃ (0.38 g, 3.59 mmol) in toluene: EtOH: H₂O (23.5 mL) was added Pd(PPh₃)₂Cl₂ (0.07 g, 0.10 mmol) purged with argon for 1 h and stirred for 48 h at 140° C. in a sealed tube. After completion of the starting material (by TLC), the reaction mass was cooled to RT, quenched with water (20 mL) and extracted with CH₂Cl₂ (2×100 mL). The combined organic extracts were washed with water (100 mL), brine (20 mL), dried over anhydrous Na₂SO₄ and concentrated under reduced pressure. The obtained crude compound was purified by column chromatography eluting with 50% EtOAc/Hexane to afford Intermediate 19b (0.3 g, 50%). TLC: 75% EtOAc/Hexane (Rf: 0.7). ¹H-NMR (DMSO d₆, 500 MHz): δ 13.17 (bs, 1H), 8.89 (s, 1H), 8.22 (d, J=7.5 Hz, 1H), 7.66 (d, J=8.5 Hz, 1H), 7.50 (s, 1H), 7.46 (t, J=8 Hz, 1H), 6.04 (s, 1H), 4.02 (t, J=9 Hz, 2H), 3.87-3.80 (m, 4H), 3.22-3.15 (m, 2H), 2.00-1.92 (m, 2H), 1.86 (d, J=13 Hz, 2H). MS: 537 [M+H].

### Step 19c: 4-(2-(1H-indazol-4-yl)-4-morpholinothieno[3,2-d]pyrimidin-6-yl)piperidin-4-ol (Intermediate 19c)

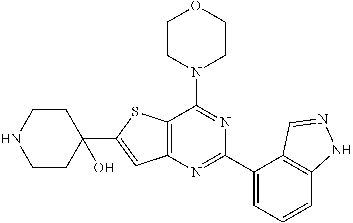

To a stirred solution of Intermediate 19b (0.15 g, 0.27 mmol) in CH₂Cl₂ (5 mL) at 0° C. was added 4M HCl in dioxane (2 mL) and allowed to RT, stirred for 4 h. After completion of the starting material (by TLC), the volatiles were removed under reduced pressure. The obtained residue was washed with EtOAc/Hexane, dried over anhydrous Na₂SO₄ and concentrated under reduced pressure to afford crude Intermediate 19c (0.1 g, 83%). This was directly used for next reaction. TLC: 100% EtOAc (Rf: 0.2).

### Step 19d: N-(2-(4-(2-(1H-indazol-4-yl)-4-morpholinothieno[3,2-d]pyrimidin-6-yl)-4-hydroxypiperidin-1-yl)-2-oxoethyl)acrylamide

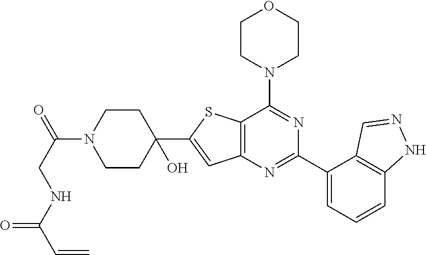

To a stirred mixture of Intermediate 19c (0.1 g, 0.22 mmol), 2-acrylamidoacetic acid (0.029 g, 0.22 mmol) in CH₂Cl₂ (5 mL) were added HATU (0.13 g, 0.33 mmol), DIPEA (0.085 g, 0.66 mmol) and stirred at RT for 10 min. Then the stirring was continued for another 5 h at RT. After the consumption of starting material (by TLC), the reaction mixture was diluted with CH₂Cl₂ (40 mL) and washed with NaHCO₃ solution (20 mL) followed by water (2×20 mL) and brine (10 mL). The combined organic extracts were dried over anhydrous Na₂SO₄ and concentrated under reduced pressure. The obtained crude compound was purified by column chromatography eluting with 5% MeOH/CH₂Cl₂ to afford II-a-134 (0.025 g, 20%). TLC: 10% MeOH/CH₂Cl₂ (Rf: 0.4). ¹H-NMR (DMSO d₆, 500 MHz): δ 13.17 (s, 1H), 8.88 (s, 1H), 8.22 (d, J=6.5 Hz, 2H), 7.66 (d, J=8.5 Hz, 1H), 7.48-7.45 (m, 2H), 6.44-6.38 (m, 1H), 6.11 (t, J=5.5 Hz, 2H), 5.61 (d, J=12 Hz, 1H), 4.32 (d, J=12.5 Hz, 1H), 4.12-4.09 (m, 2H), 4.03-4.01 (m, 4H), 3.85-3.77 (m, 5H), 3.45 (t, J=11.5 Hz, 1H), 3.08-2.91 (m, 3H), 1.93-1.91 (m, 3H). Mass: 570 [M+Na], 548 [M+H].

In a similar fashion, using an appropriate acid in the amidation step and/or a different ketone in step 19b, the following compounds were synthesized:

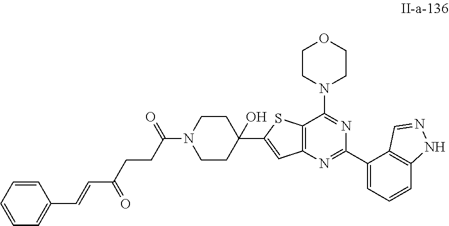

(E)-1-(4-(2-(1H-indazol-4-yl)-4-morpholinothieno[3,2-d]pyrimidin-6-yl)-4-hydroxypiperidin-1-yl)-6-phenylhex-5-ene-1,4-dione (II-a-136). MS: m/z 623.3 (ES+).

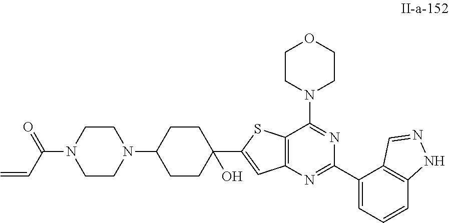

1-(4-(4-(2-(1H-indazol-4-yl)-4-morpholinothieno[3,2-d]pyrimidin-6-yl)-4-hydroxycyclohexyl)piperazin-1-yl)prop-2-en-1-one (II-a-152). TLC: 10% MeOH/CH₂Cl₂ (Rf: 0.4). ¹H-NMR (CDCl₃, 500 MHz): δ 9.02 (bs, 1H), 8.28 (s, 1H), 7.60-7.56 (m, 1H), 7.55-7.45 (m 2H), 7.36-7.38 (m, 1H), 6.60-6.51 (m, 1H), 6.32-6.25 (m, 1H), 5.71-5.66 (m, 1H), 4.10-4.04 (m, 4H), 3.95-3.90 (m, 4H), 3.70-3.54 (m, 4H), 2.64-2.60 (m, 2H), 2.53-2.41 (m, 4H), 2.17-2.14 (m, 2H), 1.96-1.78 (m, 5H). (Note: NMR data suggesting that compound is a mixture of axial & equatorial isomers) MS: 574 [M+H] HPLC Purity: 54.35+54.30 (mixture of diastereomers).

### Example 20

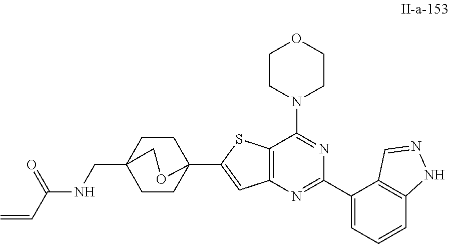

N-((1-(2-(1H-indazol-4-yl)-4-morpholinothieno[3,2-d]pyrimidin-6-yl)-2-oxabicyclo[2.2.2]octan-4-yl)methyl)acrylamide (II-a-153). The title compound was prepared according to the steps and intermediates as described below.

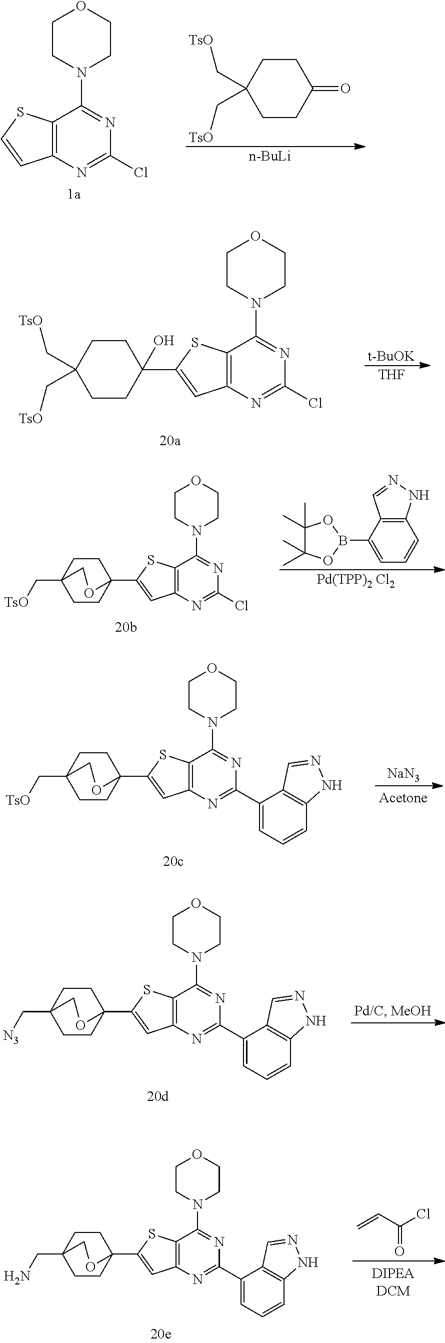

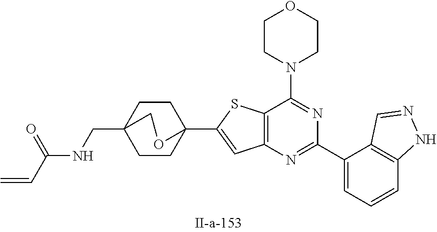

### Step 20a: (4-(2-chloro-4-morpholinothieno[3,2-d]pyrimidin-6-yl)-4-hydroxycyclohexane-1,1-diyl)bis(methylene)bis(4-methylbenzenesulfonate) (Intermediate 20a)

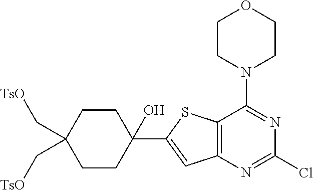

The title compound was made in a similar way as for Intermediate 19a, using Intermediate 1a and (4-oxocyclohexane-1,1-diyl)bis(methylene)bis(4-methylbenzenesulfonate). TLC: 40% EtOAc/Hexane (Rf: 0.2).

### Step 20b: (1-(2-chloro-4-morpholinothieno[3,2-d]pyrimidin-6-yl)-2-oxabicyclo[2.2.2]octan-4-yl)methyl 4-methylbenzenesulfonate (Intermediate 20b)

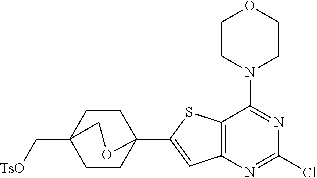

To a stirred solution of Intermediate 20a (0.6 g, 0.83 mmol) in THF (6 mL) was added potassium t-butoxide (0.18 g, 1.66 mmol) at 0° C., and the reaction mixture was refluxed for 5 h. After the consumption of starting material (by TLC), the reaction mixture was diluted with H₂O (20 mL) and extracted with EtOAc (2×50 mL). The combined organic extracts were washed with water (50 mL), brine (20 mL) were died over Na₂SO₄ and concentrated under reduced pressure to afford Intermediate 20b (0.4 g, 88%). TLC: 50% MeOH/CH₂Cl₂ (Rf: 0.6) ¹H-NMR (500 MHz CDCl₃): δ 7.78 (d, J=8.5 Hz, 2H), 7.36 (d, J=8.5 Hz, 2H), 7.0 (s, 1H), 3.99-3.97 (m, 4H), 3.85-3.80 (m, 6H), 3.76 (s, 2H), 2.46 (s, 3H), 2.19-2.04 (m, 4H), 1.81-1.76 (m, 2H), 1.67-1.55 (m, 2H). MS: 550 [M+H]

### Step 20c: (1-(2-(1H-indazol-4-yl)-4-morpholinothieno[3,2-d]pyrimidin-6-yl)-2-oxabicyclo[2.2.2]octan-4-yl)methyl 4-methylbenzenesulfonate (Intermediate 20c)

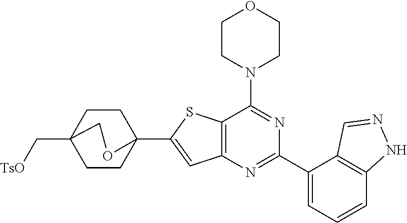

The title compound was made in a similar manner as Intermediate 19b. TLC: 70% EtOAc/Hexane (Rf: 0.3) ¹H-NMR (500 MHz CDCl₃): δ 9.00 (s, 1H), 8.26 (d, J=7.5 Hz, 1H), 8.11 (s, 1H), 7.79 (d, J=8.5 Hz, 2H), 7.59-7.55 (m, 1H), 7.37 (d, J=8.0 Hz, 2H), 7.23 (s, 1H), 4.13-4.09 (m, 6H), 3.90 3.82 (m, 4H), 3.78 (s, 2H), 2.47 (s, 3H), 2.24-2.11 (m, 4H), 1.83-1.79 (m, 2H), 1.71-1.69 (m, 2H). MS: 632 [M+H].

### Step 20d: 4-(6-(4-(azidomethyl)-2-oxabicyclo[2.2.2]octan-1-yl)-2-(1H-indazol-4-yl)thieno[3,2-d]pyrimidin-4-yl)morpholine (Intermediate 20d)

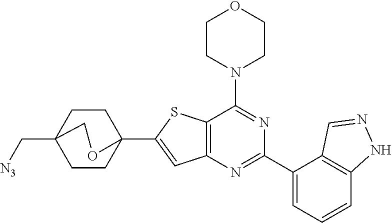

To a stirred solution of Intermediate 20c (20 mg, 0.03 mmol) in DMF (1 mL) was added NaN₃ (8.2 mg, 0.12 mmol) at room temperature and the reaction mixture was stirred at 80° C. for 12 h. After the consumption of starting material (by TLC), the reaction mixture was quenched with H₂O (2 mL) and extracted with EtOAc (2×10 mL), washed with brine (5 mL). The combined organic extracts were dried over anhydrous Na₂SO₄ and concentrated under reduced pressure to afford crude Intermediate 20d (13 mg, 86%). TLC: 70% EtOAc/Hexane (Rf: 0.4) ¹H-NMR (500 MHz CDCl₃): δ 8.99 (s, 1H), 8.26-8.20 (d, J=7.5 Hz, 1H), 7.69-7.61 (m, 1H), 7.59-7.55 (m, 1H), 7.48-7.45 (m, 1H), 4.11-4.09 (m, 4H), 3.93 (s, 2H), 3.91-3.89 (m, 4H), 3.48 (s, 2H), 2.29-2.15 (m, 4H), 1.84-1.69 (m, 4H). MS: 503 [M+H]

### Step 20e: (1-(2-(1H-indazol-4-yl)-4-morpholinothieno[3,2-d]pyrimidin-6-yl)-2-oxabicyclo[2.2.2]octan-4-yl)methanamine (Intermediate 20e)

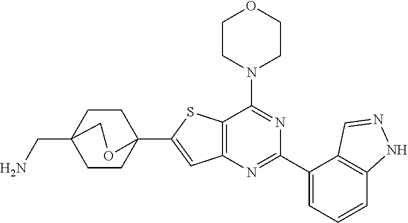

To a stirred solution of Intermediate 20d (0.3 g, 0.59 mmol) in MeOH (3 mL) was added Pd/C (30 mg), ethylene diamine (0.01 mL) and the reaction mixture was stirred at room temperature under H₂ balloon pressure for 2 h. The reaction mixture was filtered through celite bed, washed with EtOAc. The filtrate was separated, dried over anhydrous Na₂SO₄ and concentrated under reduced pressure to afford Intermediate 20e (0.25 g, 89%). TLC: 70% EtOAc/Hexane (Rf: 0.1) ¹H-NMR (500 MHz, CDCl₃): δ 9.01 (s, 1H), 8.27 (d, J=7.0 Hz, 1H), 7.59-7.26 (m, 3H), 4.11-4.09 (m, 4H), 3.93-3.89 (m, 6H), 2.55 (s, 2H), 2.30-2.14 (m, 4H), 1.79-1.70 (m, 4H).

### Step 20f: N-((1-(2-(1H-indazol-4-yl)-4-morpholinothieno[3,2-d]pyrimidin-6-yl)-2-oxabicyclo[2.2.2]octan-4-yl)methyl)acrylamide (II-a-153)

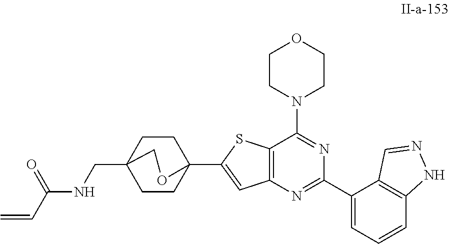

To a stirred solution of Intermediate 20e (0.07 g, 0.14 mmol) in CH₂Cl₂ (2 mL) was added DIPEA (37 mg, 0.28 mmol) at RT. The resultant reaction mixture was cooled to −10° C. followed by the addition of acryloyl chloride (13 mg, 0.14 mmol) and the reaction mixture was stirred for 5 min. After the consumption of starting material (by TLC), the reaction mixture was triturated with H₂O (2×10 mL) and extracted with CH₂Cl₂. The combined organic layer dried over anhydrous Na₂SO₄ and concentrated under reduced pressure. The obtained crude compound was purified by silica gel column chromatography eluting with 5% MeOH/CH₂Cl₂ to afford II-a-153 (10 mg). TLC: 10% MeOH/CH₂Cl₂ (Rf: 0.2). ¹H-NMR (500 MHz CDCl₃₊ CD₃OD): δ 8.88 (s, 1H), 8.18 (d, J=7.5 Hz, 1H), 7.61 (d, J=8.0 Hz, 1H), 7.60 (t, J=8.0 Hz, 1H), 7.26 (s, 1H), 6.30 (d, J=17.0 Hz, 1H), 6.19-6.14 (m, 1H), 5.68 (d, J=10.5 Hz, 1H), 4.11-4.09 (m, 4H), 3.92-3.90 (m, 6H), 3.19 (s, 2H), 2.26-2.16 (m, 4H), 1.81-1.76 (m, 4H). MS: 530 [M+H].

In a similar fashion, using an appropriate acid in the amide formation step, the following compounds were synthesized:

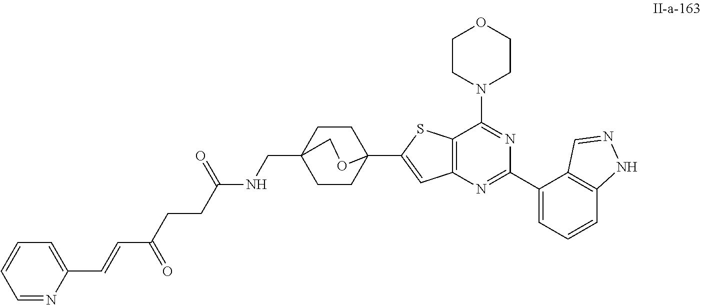

(E)-N-((1-(2-(1H-indazol-4-yl)-4-morpholinothieno[3,2-d]pyrimidin-6-yl)-2-oxabicyclo[2.2.2]octan-4-yl)methyl)-4-oxo-6-(pyridin-2-yl)hex-5-enamide (II-a-163). ¹H-NMR (500 MHz, CDCl₃+CD₃OD): δ 8.89 (s, 1H), 8.64 (d, J=5 Hz, 1H), 8.19 (d, J=7.0 Hz, 1H), 7.77 (t, J=8.0 Hz, 1H), 7.63-7.60 (m, 2H), 7.53-7.48 (m, 2H), 7.25 (s, 1H), 7.10 (d, J=16 Hz, 1H), 6.73 (t, J=6.0 Hz, 1H), 4.10 (t, J=4.5 Hz, 4H), 3.91-3.90 (m, 6H), 3.12-3.10 (m, 4H), 2.56 (t, J=6.5 Hz, 2H), 2.18-2.05 (m, 4H), 1.80-1.75 (m, 4H). MS: 665 [M+H].

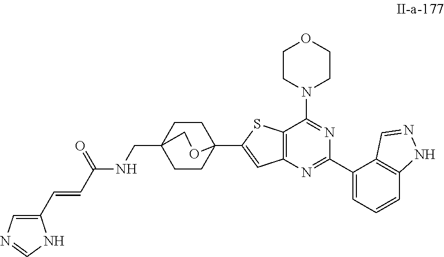

(E)-N-((1-(2-(1H-indazol-4-yl)-4-morpholinothieno[3,2-d]pyrimidin-6-yl)-2-oxabicyclo[2.2.2]octan-4-yl)methyl)-3-(1H-imidazol-5-yl)acrylamide (II-a-177). MS: m/z 597.0 (ES+).

### Example 21

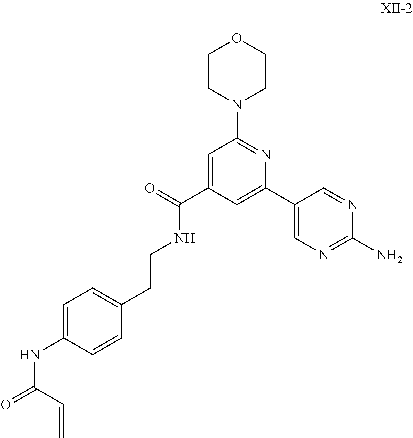

N-(4-acrylamidophenethyl)-2-(2-aminopyrimidin-5-yl)-6-morpholinoisonicotinamide (XII-2): The title compound was prepared according to the steps and intermediates as described below.

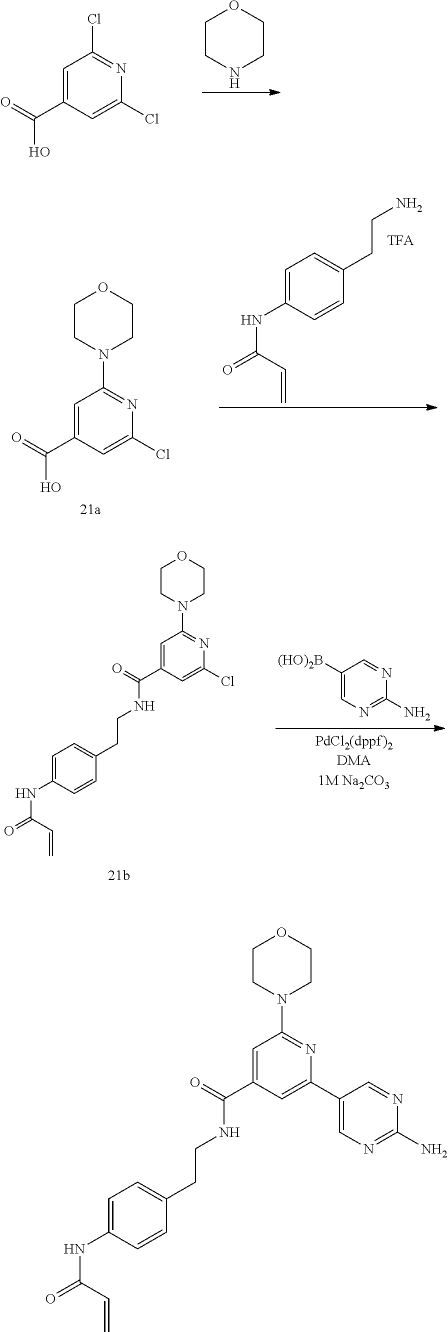

### Step 21a: 2-chloro-6-morpholinoisonicotinic acid (Intermediate 21a)

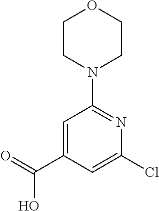

2,6-dichloroisonicotinic acid (1.92 g, 10 mmol), 1 mL of morpholine (11.5 mmol), and 3.5 mL of DIPEA (21.2 mmol) in 10 mL of DMA (N,N-dimethylacetamide) were heated at 150° C. under microwave condition for 60 min. The excess amount of solvent was then evaporated under reduced pressure, and the residue was suspended in 10 mL of acetonitrile. 10 mL of 1.0 N aqueous HCl was added for neutralization, the pale white solid was collected filtration. Additional portion of product was also obtained from mother liquor, which gave total 1.59 g of pale white solid as desired product (Y: 65%). LC-MS: m/z 243.2 (ES+).

### Step 21b: N-(4-acrylamidophenethyl)-2-chloro-6-morpholinoisonicotinamide (Intermediate 21b)

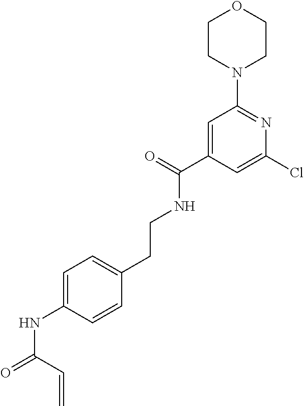

The title intermediate was prepared in the same way as described in Example 16. MS: m/z 415.1 (ES+).

### Step 21c: N-(4-acrylamidophenethyl)-2-(2-aminopyrimidin-5-yl)-6-morpholinoisonicotinamide (XII-2)

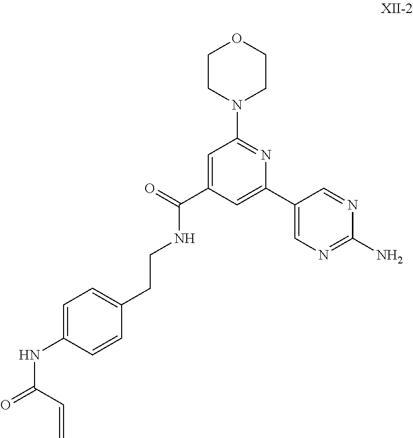

Under Ar, a mixture of Intermediate 21b (11 mg, 26 umol), 2-aminopyrimidine 5-boronic acid (5 mg; 36 umol), PdCl₂(dppf)₂ (1 mg, 5% mol), in 600 uL of DMA and 100 uL of 1 M aqueous Na₂CO₃ was heated at 135° C. for 60 min in CEM microwave. The resulting black mixture was filtrated, and purified by prep-HPLC, giving 8 mg of desired product as white solid. LC-MS: m/z 474.0 (ES+).

In a similar fashion, using an appropriate boronic acid and/or amine, the following compounds were made:

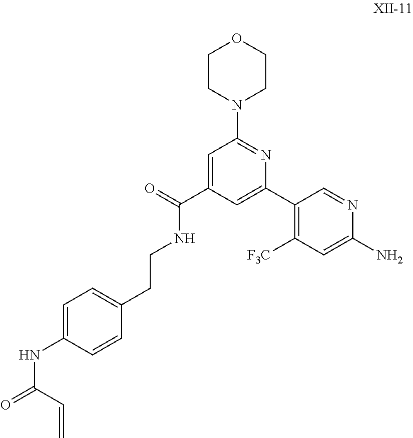

N-(4-acrylamidophenethyl)-6′-amino-6-morpholino-4′-(trifluoromethyl)-2,3′-bipyridine-4-carboxamide (XII-11). MS: m/z 541.1 (ES+).

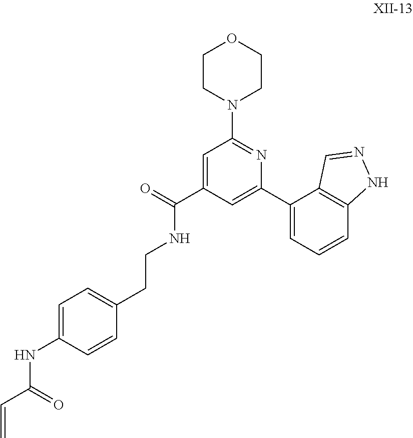

N-(4-acrylamidophenethyl)-2-(1H-indazol-4-yl)-6-morpholinoisonicotinamide (XII-13). MS: m/z 497.1 (ES+).

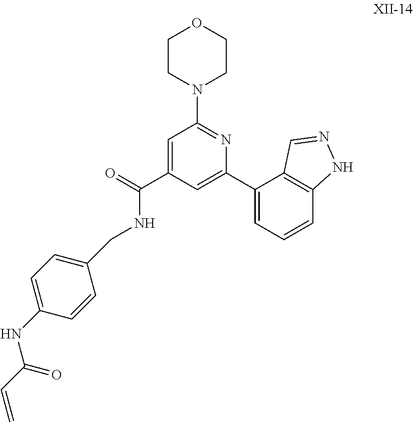

N-(4-acrylamidobenzyl)-2-(1H-indazol-4-yl)-6-morpholinoisonicotinamide (XII-14). MS: m/z 483.2 (ES+).

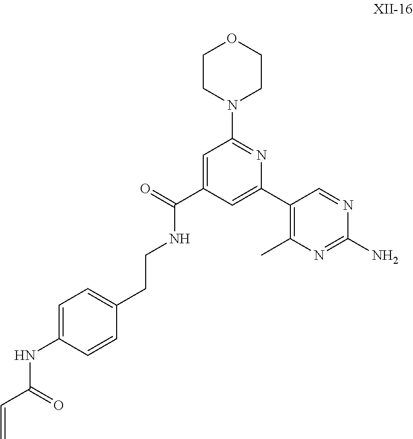

N-(4-acrylamidophenethyl)-2-(2-amino-4-methylpyrimidin-5-yl)-6-morpholinoisonicotinamide (XII-16). MS: m/z 488.3 (ES+).

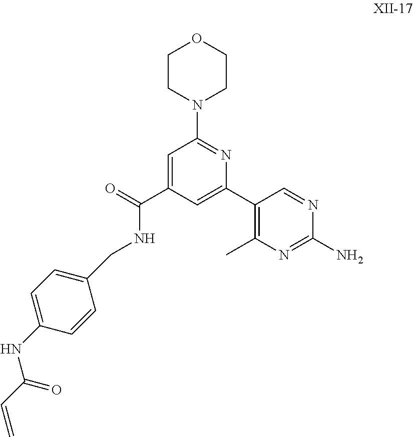

N-(4-acrylamidobenzyl)-2-(2-amino-4-methylpyrimidin-5-yl)-6-morpholinoisonicotinamide (XII-17). MS: m/z 474.1 (ES+).

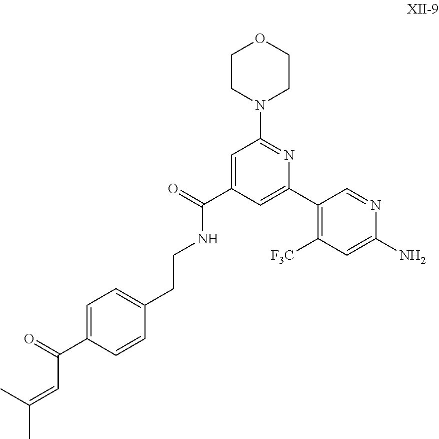

6′-amino-N-(4-(3-methylbut-2-enoyl)phenethyl)-6-morpholino-4′-(trifluoromethyl)-2,3′-bipyridine-4-carboxamide (XII-9). MS: m/z 554.2 (ES+).

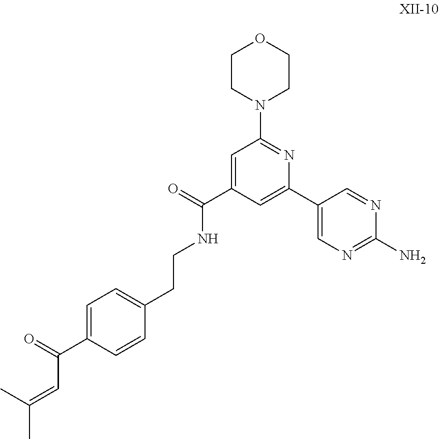

2-(2-aminopyrimidin-5-yl)-N-(4-(3-methylbut-2-enoyl)phenethyl)-6-morpholinoisonicotinamide (XII-10). MS: m/z 487.1 (ES+).

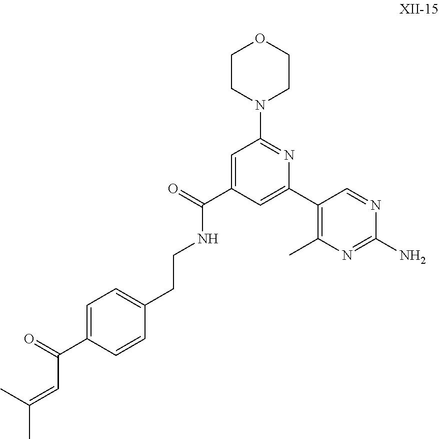

2-(2-amino-4-methylpyrimidin-5-yl)-N-(4-(3-methylbut-2-enoyl)phenethyl)-6-morpholinoisonicotinamide (XII-15). MS: m/z 501.2 (ES+).

### Example 22

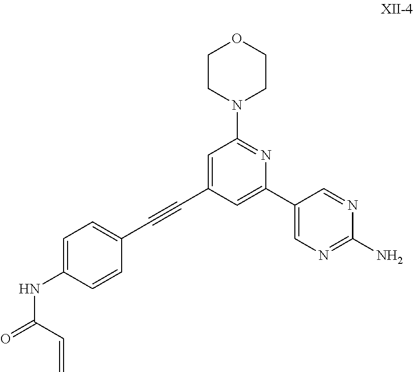

N-(4-((2-(2-aminopyrimidin-5-yl)-6-morpholinopyridin-4-yl)ethynyl)phenyl)acrylamide (XII-4): The title compound was synthesized according to the following intermediates and steps as described below.

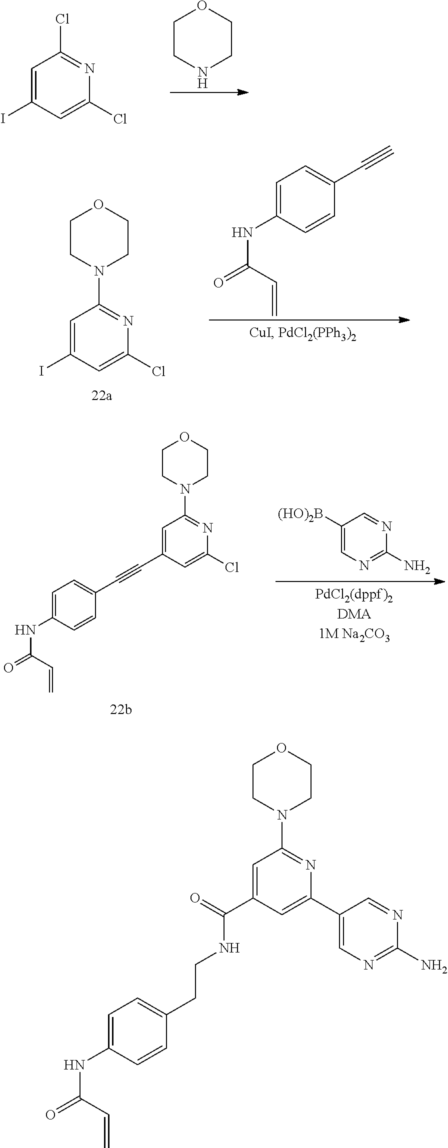

### Step 22a: 4-(6-chloro-4-iodopyridin-2-yl)morpholine (Intermediate 22a)

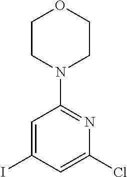

2,6-dichloro-4-iodopyridine (2.0 g, 7.3 mmol), morpholine (700 uL, 8.0 mmol) and 1.5 mL of DIPEA in 15 mL of anhydrous dioxane were heated at 120° C. for 24 hr. After concentration and regular aqueous workup with ethyl acetate-water, the reaction mixture was subject to column chromatography on silica gel, eluting with heptane/ethyl acetate (v/v 6/1), giving 1.74 g of desired product as white crystal. MS: m/z 325.0 (ES+).

### Step 22b: N-(4-((2-chloro-6-morpholinopyridin-4-yl)ethynyl)phenyl)acrylamide (Intermediate 22b)

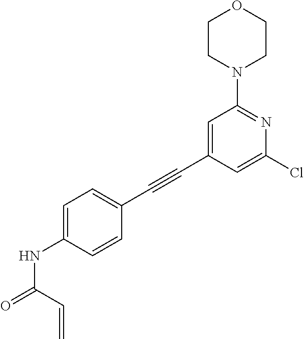

Under Ar, Intermediate 22a (36 mg, 110 umol), N-(4-ethynylphenyl)acrylamide (20 mg, 120 umol, readily available from 4-ethynylaniline and acryloyl chloride), PdCl₂(PPh₃)₂ (4 mg, 5% mol), CuI (2 mg, 10% mol), 40 uL of DIPEA in 1 mL of DMA were heated at 80° C. overnight. After workup with ethyl acetate and water, the reaction mixture was subject to column chromatography on silica gel, eluting with heptanes/ethyl acetate (v/v 3/2), giving 32 mg of desired product as white solid. MS: m/z 368.1 (ES+).

### Step 22c: N-(4-((2-(2-aminopyrimidin-5-yl)-6-morpholinopyridin-4-yl)ethynyl)phenyl)acrylamide (XII-4)

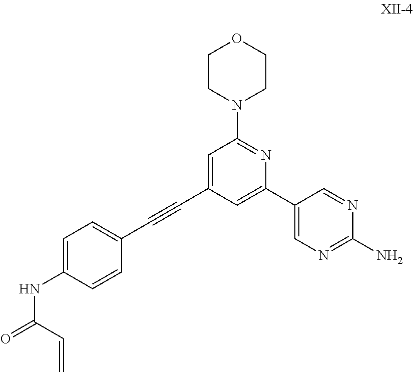

The title compound was prepared using Intermediate 22b via Suzuki coupling as described in Example 21. MS: m/z 427.1 (ES+).

In similar fashion, using an appropriate boronic acid and/or appropriate alkyne, the following compounds were prepared:

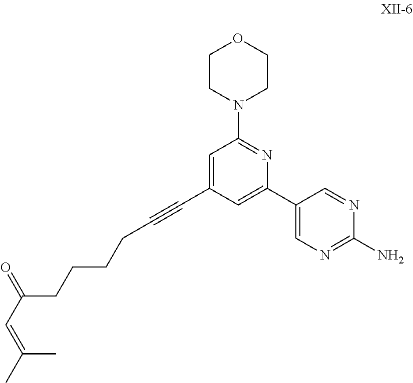

10-(2-(2-aminopyrimidin-5-yl)-6-morpholinopyridin-4-yl)-2-methyldec-2-en-9-yn-4-one (XII-6). MS: m/z 420.2 (ES+).

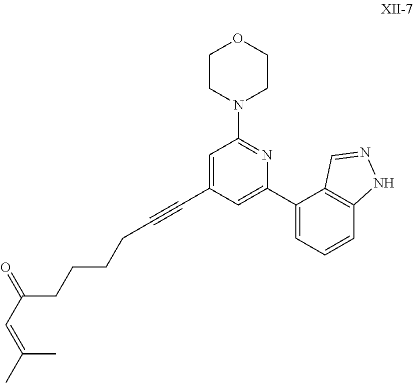

10-(2-(1H-indazol-4-yl)-6-morpholinopyridin-4-yl)-2-methyldec-2-en-9-yn-4-one (XII-7). MS: m/z 443.1 (ES+).

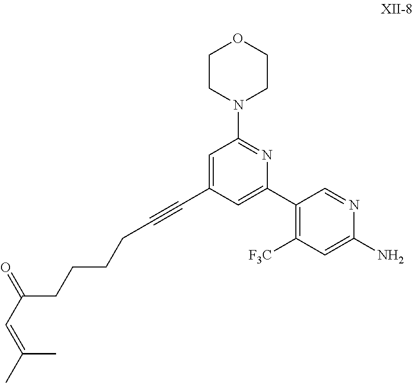

10-(6′-amino-6-morpholino-4′-(trifluoromethyl)-2,3′-bipyridin-4-yl)-2-methyldec-2-en-9-yn-4-one (XII-8). MS: m/z 487.1 (ES+).

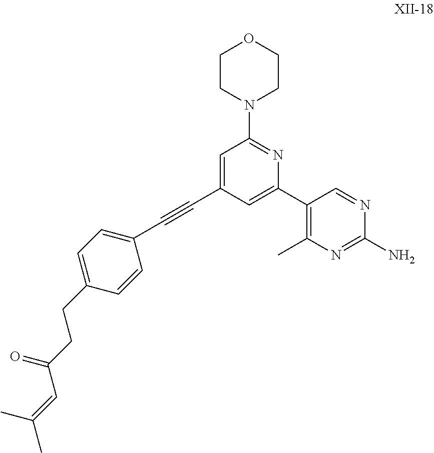

1-(4-((2-(2-amino-4-methylpyrimidin-5-yl)-6-morpholinopyridin-4-yl)ethynyl)phenyl)-5-methylhex-4-en-3-one (XII-18). MS: m/z 482.1 (ES+).

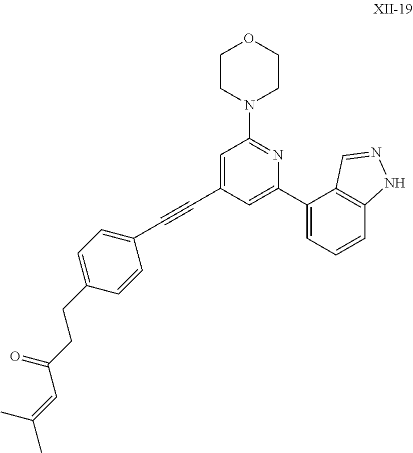

1-(4-((2-(1H-indazol-4-yl)-6-morpholinopyridin-4-yl)ethynyl)phenyl)-5-methylhex-4-en-3-one (XII-19). MS: m/z 491.1 (ES+).

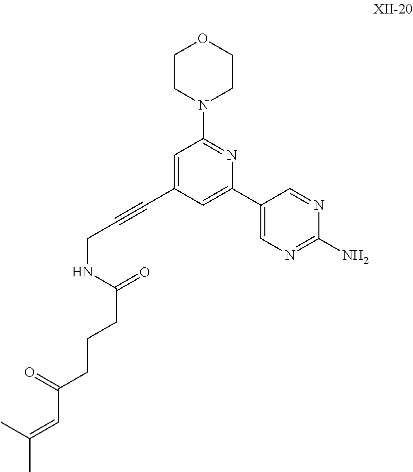

N-(3-(2-(2-aminopyrimidin-5-yl)-6-morpholinopyridin-4-yl)prop-2-ynyl)-7-methyl-5-oxooct-6-enamide (XII-20). MS: m/z 463.2 (ES+).

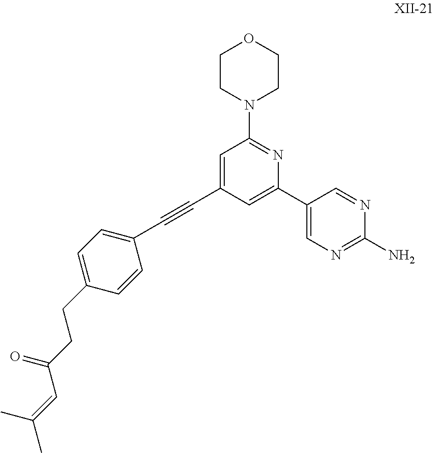

1-(4-((2-(2-aminopyrimidin-5-yl)-6-morpholinopyridin-4-yl)ethynyl)phenyl)-5-methylhex-4-en-3-one (XII-21). MS: m/z 468.1 (ES+).

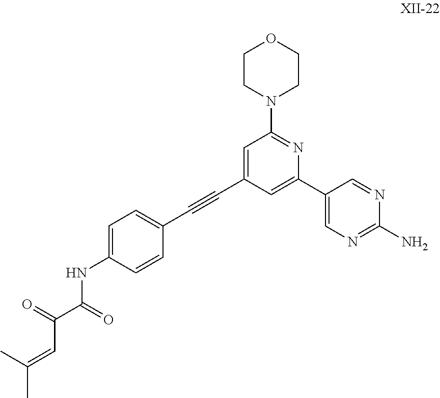

N-(4-((2-(2-aminopyrimidin-5-yl)-6-morpholinopyridin-4-yl)ethynyl)phenyl)-4-methyl-2-oxopent-3-enamide (XII-22). MS: m/z 483.1 (ES+).

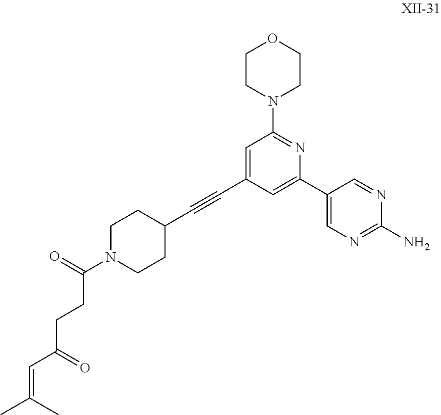

1-(4-((2-(2-aminopyrimidin-5-yl)-6-morpholinopyridin-4-yl)ethynyl)piperidin-1-yl)-6-methylhept-5-ene-1,4-dione (XII-31). MS: m/z 503.3 (ES+).

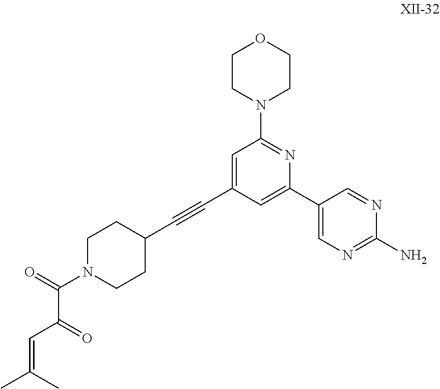

1-(4-((2-(2-aminopyrimidin-5-yl)-6-morpholinopyridin-4-yl)ethynyl)piperidin-1-yl)-4-methylpent-3-ene-1,2-dione (XII-32). MS: m/z 475.2 (ES+).

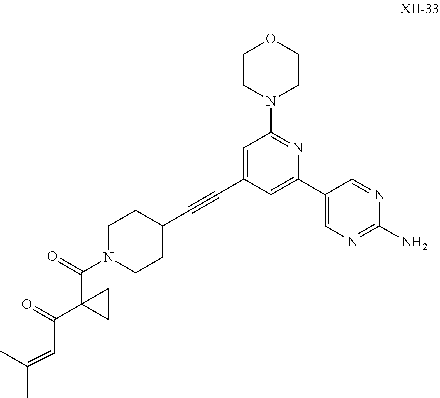

1-(1-(4-((2-(2-aminopyrimidin-5-yl)-6-morpholinopyridin-4-yl)ethynyl)piperidine-1-carbonyl)cyclopropyl)-3-methylbut-2-en-1-one (XII-33). MS: m/z 515.2 (ES+).

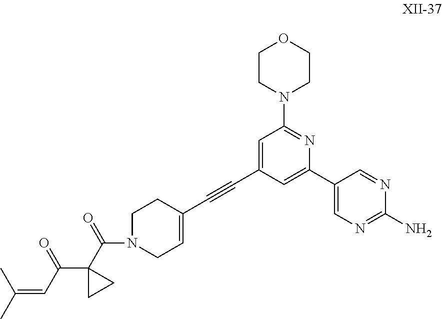

1-(1-(4-((2-(2-aminopyrimidin-5-yl)-6-morpholinopyridin-4-yl)ethynyl)-1,2,3,6-tetrahydropyridine-1-carbonyl)cyclopropyl)-3-methylbut-2-en-1-one (XII-37). MS: m/z 513.2 (ES+).

### Example 23

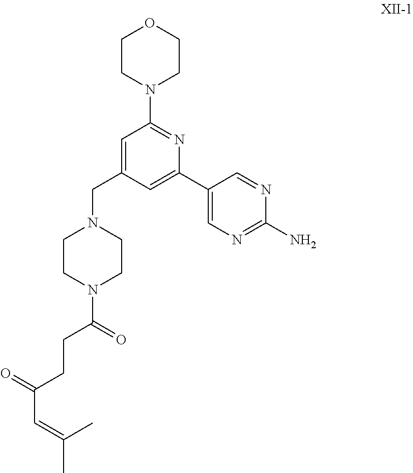

1-(4-((2-(2-aminopyrimidin-5-yl)-6-morpholinopyridin-4-yl)methyl)piperazin-1-yl)-6-methylhept-5-ene-1,4-dione (XII-1). The title compound was synthesized according to the following intermediates and stets as described below.

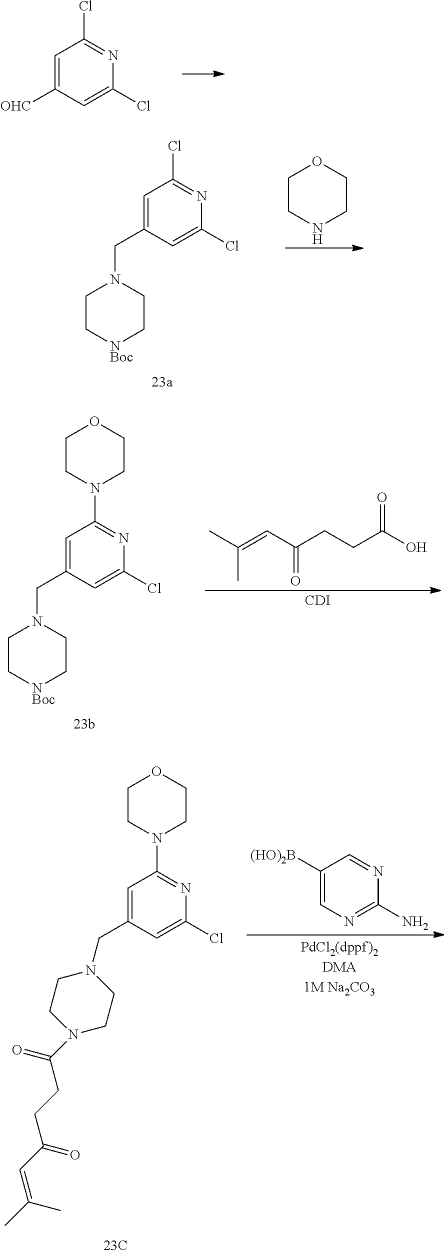

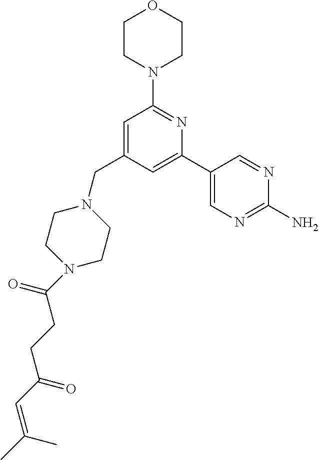

### Step 23a: tert-butyl 4-((2,6-dichloropyridin-4-yl)methyl)piperazine-1-carboxylate (Intermediate 23a)

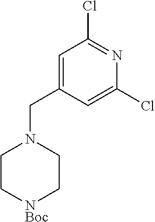

2,6-dichloroisonicotinaldehyde (106 mg, 0.6 mmol), N-Boc-piperizane (112 mg, 0.6 mmol) and 320 mg of NaBH(OAc)₃ powder was stirred in 5 mL of dichloromethane at room temperature for 1 hr. 3 mL of saturated NaHCO₃ aqueous solution was added, the reaction mixture was stirred for additional 30 min. After regular aqueous workup with dichloromethane-water, the reaction mixture was subject to column chromatography on silica gel, eluting with heptane/ethyl acetate (v/v 3/1), giving 150 mg of desired product as colorless oil. MS: m/z 346.0 (ES+); 290.0 (M-Bu-t, ES+).

### Step 23b: tert-butyl 4-((2-chloro-6-morpholinopyridin-4-yl)methyl)piperazine-1-carboxylate (Intermediate 23b)

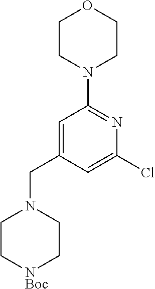

A mixture of Intermediate 23a (75 mg, 0.22 mmol), morpholine (60 uL, ˜3 equiv) in 3 mL of dioxane was heated at 115° C. overnight. After removing the solvent completely, the residue was purified by column chromatography on silica gel, with heptane/ethyl acetate (v/v 1/1) as eluent, giving desired Intermediate 23b (62 mg, 71%). MS: m/z 397.1 (ES+).

### Step 23c: 1-(4-((2-chloro-6-morpholinopyridin-4-yl)methyl)piperazin-1-yl)-6-methylhept-5-ene-1,4-dione (Intermediate 23c)

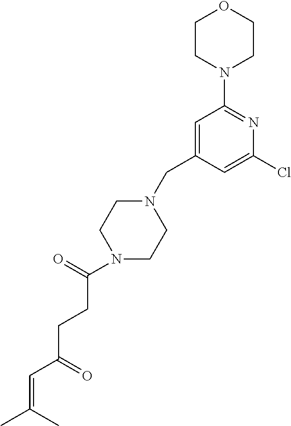

The deprotection of Boc group on Intermediate 23b was carried out using 2 mL of 4 N HCl in dioxane in 1.5 mL of a mixed solvent (CH₂Cl₂/MeOH, v/v 2/1) at room temperature for 1 hr. After removing the solvent, the residue was dried completely and used directly for following step. MS: m/z 297.0 (ES+)

6-methyl-4-oxohept-5-enoic acid (10 mg, 64 umol) and carbonyl diimidazole (10.5 mg, 64 umol) was stirred in 1 mL of DMA for 1 hr, before 18 mg of de-boc intermediate obtained above and 100 uL of DIPEA were added in. The reaction mixture was stirred at room temperature overnight, then purified by prep-HPLC, giving 15 mg Intermediate 23c. MS: m/z 435.2 (ES+).

### Step 23d: 1-(4-((2-(2-aminopyrimidin-5-yl)-6-morpholinopyridin-4-yl)methyl)piperazin-1-yl)-6-methylhept-5-ene-1,4-dione (XII-1)

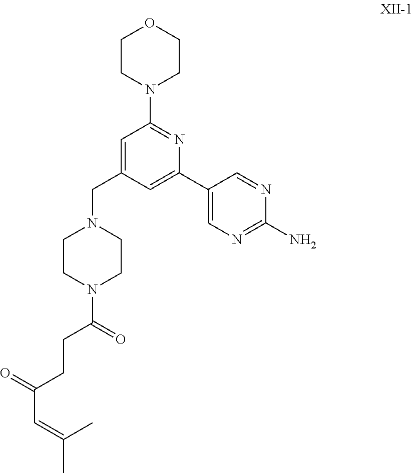

The title compound was prepared in the same way as described in Example 21 via Suzuki coupling with Intermediate 23c. MS: m/z 494.1 (ES+).

In a similar fashion, the following compound was prepared:

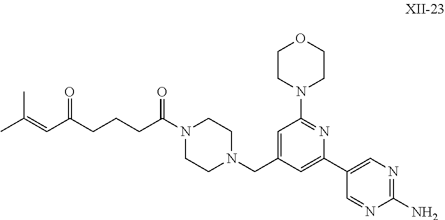

1-(4-((2-(2-aminopyrimidin-5-yl)-6-morpholinopyridin-4-yl)methyl)piperazin-1-yl)-7-methyloct-6-ene-1,5-dione (XII-23). MS: m/z 508.2 (ES+).

### Example 24

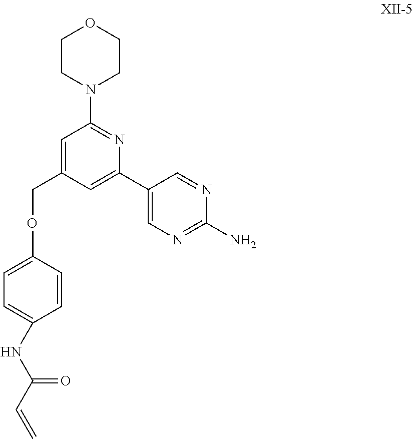

N-(4-((2-(2-aminopyrimidin-5-yl)-6-morpholinopyridin-4-yl)methoxy)phenyl)acrylamide (XII-5). The title compound was synthesized through the steps and intermediates as described below.

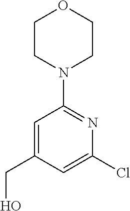

(2-chloro-6-morpholinopyridin-4-yl)methanol. The title intermediate was prepared in a similar way as described for Intermediate 21a, by reacting morpholine with (2,6-dichloro-pyridin-4-yl)methanol in dioxane. MS: m/z 229.1 (ES+).

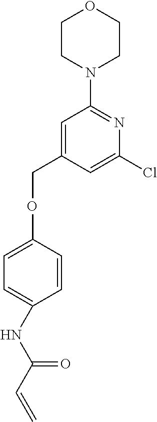

N-(4-((2-chloro-6-morpholinopyridin-4-yl)methoxy)phenyl)acrylamide. The title intermediate was prepared by the alcohol intermediate obtained above and N-(4-hydroxyphenyl)acrylamide via a standard Mitsunobu reaction. MS: m/z 374.1 (ES+).

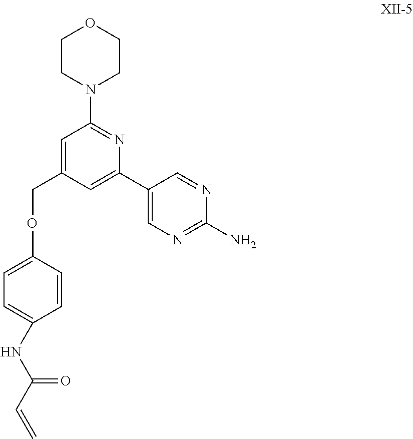

N-(4-((2-(2-aminopyrimidin-5-yl)-6-morpholinopyridin-4-yl)methoxy)phenyl)acrylamide (XII-5). The title compound was prepared in the same way as described in Example 21 via Suzuki coupling with the intermediate obtained above. MS: m/z 433.1 (ES+).

### Example 25

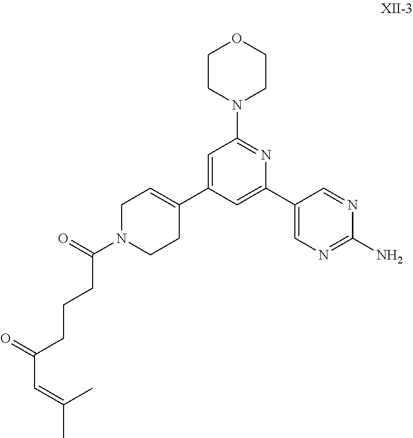

1-(4-(2-(2-aminopyrimidin-5-yl)-6-morpholinopyridin-4-yl)-5,6-dihydropyridin-1(2H)-yl)-7-methyloct-6-ene-1,5-dione (XII-3). The title compound was synthesized through the steps and intermediates as described below.

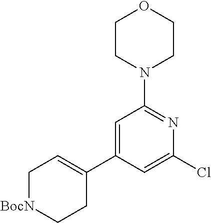

tert-butyl 4-(2-chloro-6-morpholinopyridin-4-yl)-5,6-dihydropyridine-1(2H)-carboxylate. The title intermediate was prepared using Intermediate 21a and N-Boc-tetrahydropyridine-4-boronic ester through Suzuki coupling. MS: m/z 380.1 (ES+).

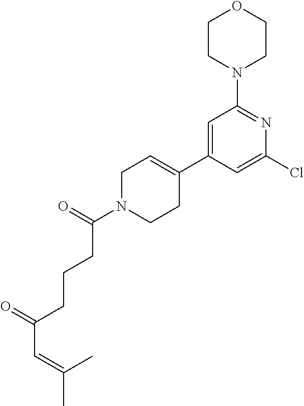

1-(4-(2-chloro-6-morpholinopyridin-4-yl)-5,6-dihydropyridin-1(2H)-yl)-7-methyloct-6-ene-1,5-dione. The title intermediate was prepared via amidation as described in Example 23 using the intermediate prepared from previous step. MS: m/z 432.1 (ES+).

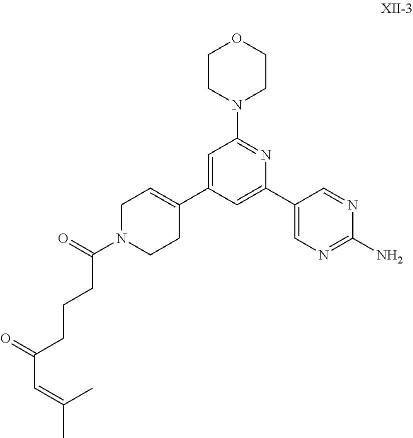

1-(4-(2-(2-aminopyrimidin-5-yl)-6-morpholinopyridin-4-yl)-5,6-dihydropyridin-1(2H)-yl)-7-methyloct-6-ene-1,5-dione (XII-3). The title compound was prepared in the same way as described in Example 21 via Suzuki coupling with the intermediate obtained above. MS: m/z 491.1 (ES+).

In a similar fashion, using different boronic acids and/or various acids in final HATU coupling, the following compounds were synthesized.

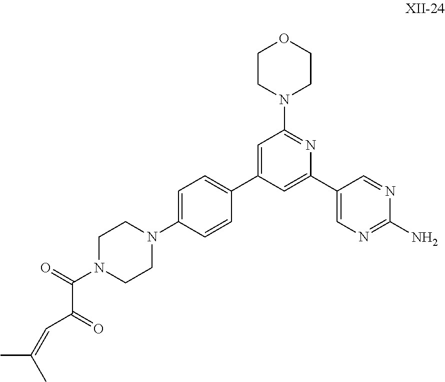

1-(4-(4-(2-(2-aminopyrimidin-5-yl)-6-morpholinopyridin-4-yl)phenyl)piperazin-1-yl)-4-methylpent-3-ene-1,2-dione (XII-24). MS: m/z 528.2 (ES+).

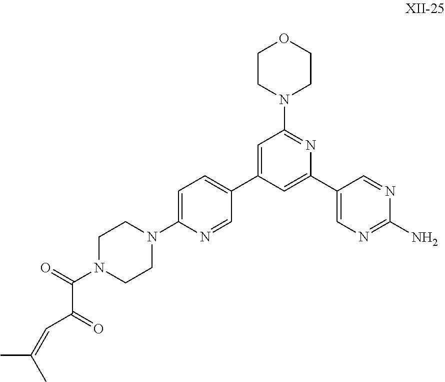

1-(4-(2′-(2-aminopyrimidin-5-yl)-6′-morpholino-3,4′-bipyridin-6-yl)piperazin-1-yl)-4-methylpent-3-ene-1,2-dione (XII-24). MS: m/z 529.2 (ES+).

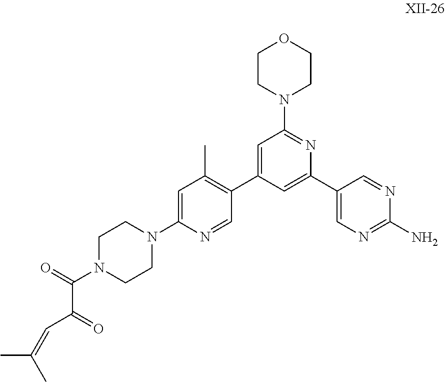

1-(4-(2′-(2-aminopyrimidin-5-yl)-4-methyl-6′-morpholino-3,4′-bipyridin-6-yl)piperazin-1-yl)-4-methylpent-3-ene-1,2-dione (XII-26). MS: m/z 543.2 (ES+).

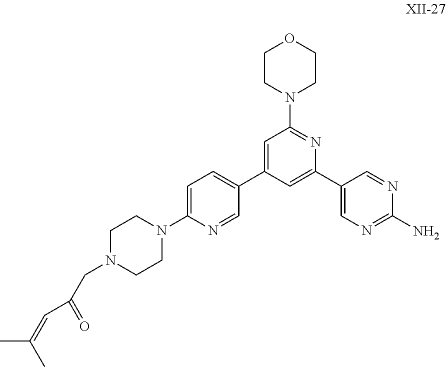

1-(4-(2′-(2-aminopyrimidin-5-yl)-6′-morpholino-3,4′-bipyridin-6-yl)piperazin-1-yl)-4-methylpent-3-en-2-one (XII-27). MS: m/z 515.2 (ES+).

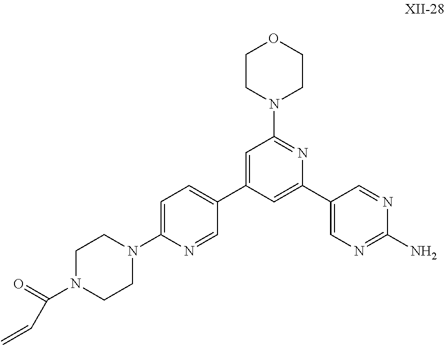

1-(4-(2′-(2-aminopyrimidin-5-yl)-6′-morpholino-3,4′-bipyridin-6-yl)piperazin-1-yl)prop-2-en-1-one (XII-28). LC-MS: m/z 473.1 (ES+).

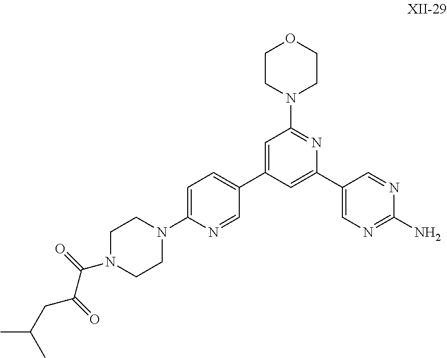

1-(4-(2′-(2-aminopyrimidin-5-yl)-6′-morpholino-3,4′-bipyridin-6-yl)piperazin-1-yl)-4-methylpentane-1,2-dione (XII-29). MS: m/z 531.2 (ES+).

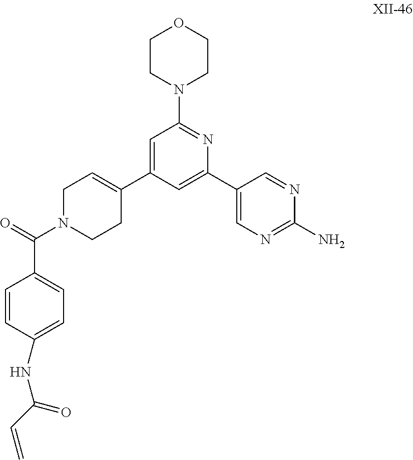

N-(4-(4-(2-(2-aminopyrimidin-5-yl)-6-morpholinopyridin-4-yl)-1,2,3,6-tetrahydropyridine-1-carbonyl)phenyl)acrylamide (XII-46). MS: m/z 512.3 (ES+).

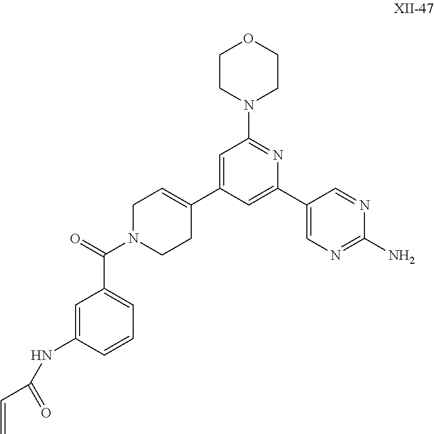

N-(3-(4-(2-(2-aminopyrimidin-5-yl)-6-morpholinopyridin-4-yl)-1,2,3,6-tetrahydropyridine-1-carbonyl)phenyl)acrylamide (XII-47). MS: m/z 512.3 (ES+).

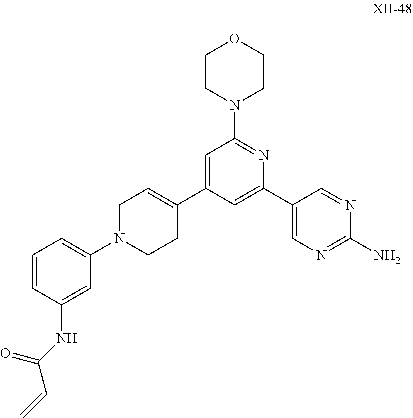

N-(3-(4-(2-(2-aminopyrimidin-5-yl)-6-morpholinopyridin-4-yl)-5,6-dihydropyridin-1(2H)-yl)phenybacrylamide (XII-48). MS: m/z 484.2 (ES+).

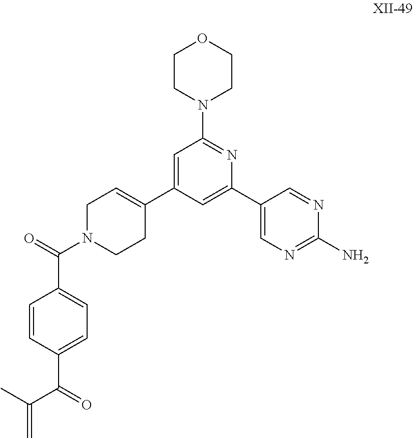

1-(4-(4-(2-(2-aminopyrimidin-5-yl)-6-morpholinopyridin-4-yl)-1,2,3,6-tetrahydropyridine-1-carbonyl)phenyl)-2-methylprop-2-en-1-one (XII-49). MS: m/z 511.2 (ES+).

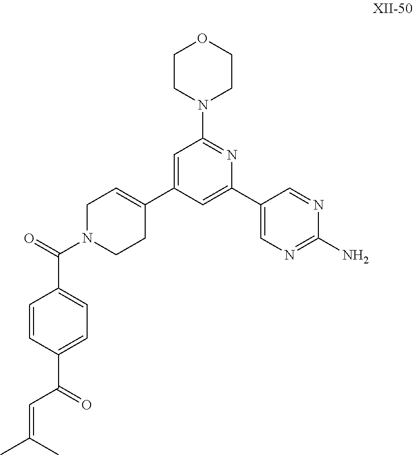

1-(4-(4-(2-(2-aminopyrimidin-5-yl)-6-morpholinopyridin-4-yl)-1,2,3,6-tetrahydropyridine-1-carbonyl)phenyl)-3-methylbut-2-en-1-one (XII-50). MS: m/z 525.2 (ES+).

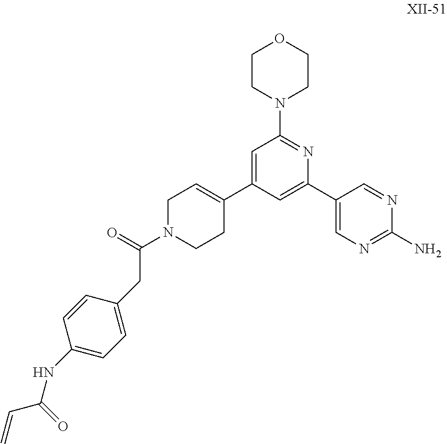

N-(4-(2-(4-(2-(2-aminopyrimidin-5-yl)-6-morpholinopyridin-4-yl)-5,6-dihydropyridin-1(2H)-yl)-2-oxoethyl)phenyl)acrylamide (XII-51). MS: m/z 526.2 (ES+).

### Example 26

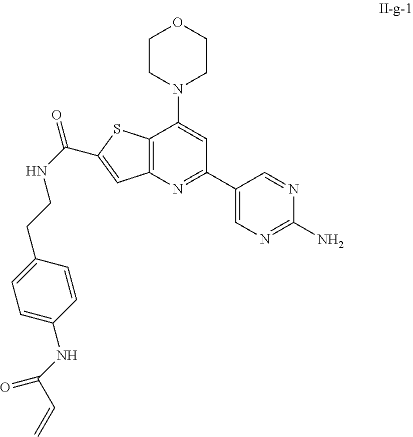

N-(4-acrylamidophenethyl)-5-(2-aminopyrimidin-5-yl)-7-morpholinothieno[3,2-b]pyridine-2-carboxamide (II-g-1). The title compound was synthesized in the same way as for II-a-154, starting from 5,7-dichlorothieno[3,2-b]pyridine instead of 2,4-dichlorothieno[3,2-d]pyrimidine. MS: m/z 531.0 (ES+).

Similarly, using 5,7-dichlorothieno[3,2-b]pyridine in place of 2,4-dichlorothieno[3,2-d]pyrimidine as starting material, the following compounds were synthesized.

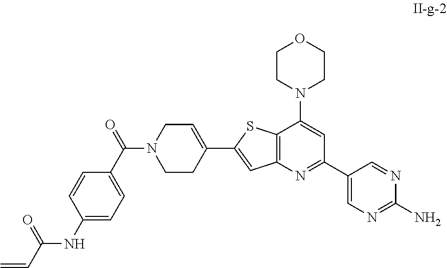

N-(4-(4-(5-(2-aminopyrimidin-5-yl)-7-morpholinothieno[3,2-b]pyridin-2-yl)-1,2,3,6-tetrahydropyridine-1-carbonyl)phenyl)acrylamide (II-g-2). The title compound was synthesized in the similar way of II-a-156 as described in Example 8. MS: m/z 568.1 (ES+).

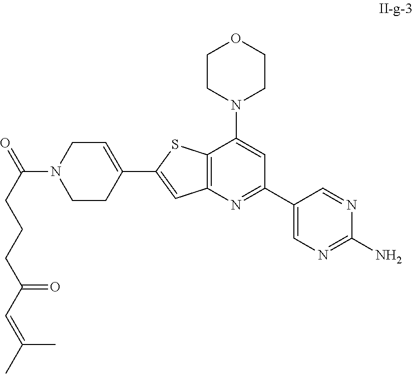

1-(4-(5-(2-aminopyrimidin-5-yl)-7-morpholinothieno[3,2-b]pyridin-2-yl)-5,6-dihydropyridin-1(2H)-yl)-7-methyloct-6-ene-1,5-dione (II-g-3). MS: m/z 547.1 (ES+).

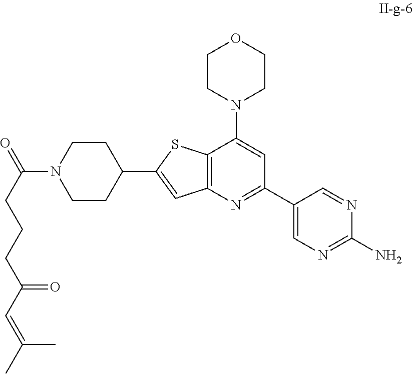

1-(4-(5-(2-aminopyrimidin-5-yl)-7-morpholinothieno[3,2-b]pyridin-2-yl)piperidin-1-yl)-7-methyloct-6-ene-1,5-dione (II-g-6). MS: m/z 549.2 (ES+).

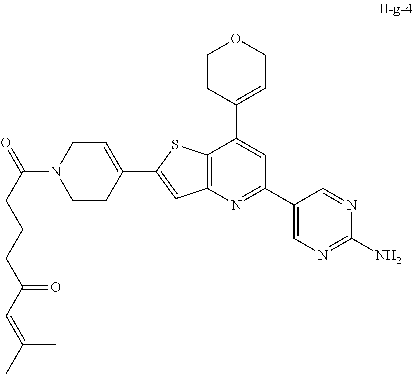

1-(4-(5-(2-aminopyrimidin-5-yl)-7-(3,6-dihydro-2H-pyran-4-yl)thieno[3,2-b]pyridin-2-yl)-5,6-dihydropyridin-1(2H)-yl)-7-methyloct-6-ene-1,5-dione (II-g-4). The title compound was synthesized in the similar way of II-a-169 as described in Example 8. MS: m/z 544.1 (ES+).

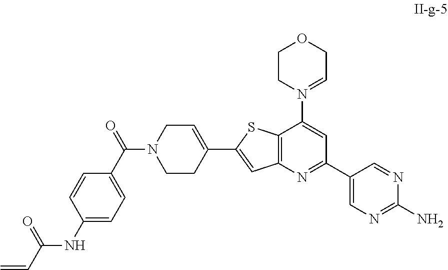

N-(4-(4-(5-(2-aminopyrimidin-5-yl)-7-(3,6-dihydro-2H-pyran-4-yl)thieno[3,2-b]pyridin-2-yl)-1,2,3,6-tetrahydropyridine-1-carbonyl)phenyl)acrylamide (II-g-5). The title compound was synthesized in the similar way of II-a-4 as described in Example 8. MS: m/z 544.1 (ES+).

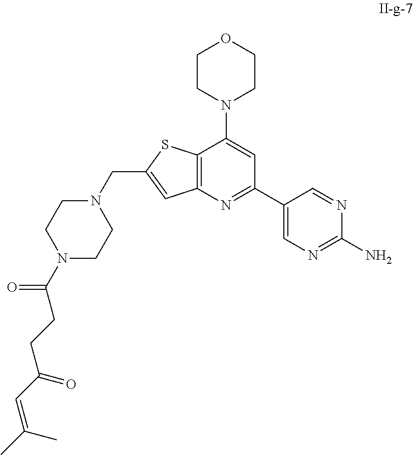

1-(4-((5-(2-aminopyrimidin-5-yl)-7-morpholinothieno[3,2-b]pyridin-2-yl)methyl)piperazin-1-yl)-6-methylhept-5-ene-1,4-dione (II-g-7). The title compound was prepared in the similar way of II-a-3 as described in Example 2. MS: m/z 550.1 (ES+).

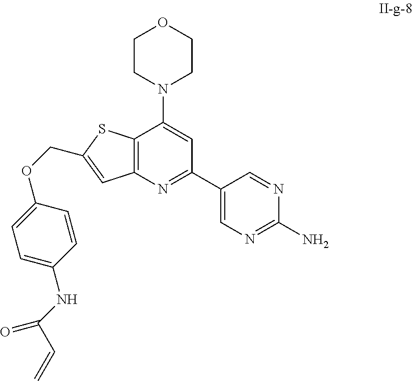

N-(4-((5-(2-aminopyrimidin-5-yl)-7-morpholinothieno[3,2-b]pyridin-2-yl)methoxy)phenyl)acrylamide (II-g-8). The title compound was prepared in the similar way of II-a-172 as described in Example 6. MS: m/z 489.0 (ES+).

### Example 27

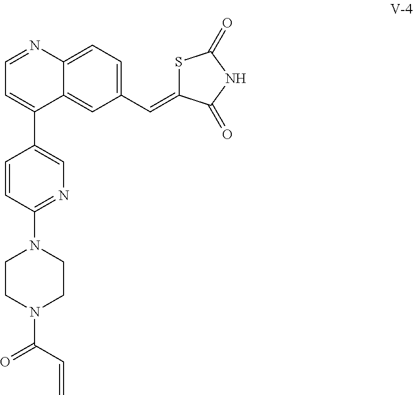

(Z)-5-((4-(6-(4-acryloylpiperazin-1-yl)pyridin-3-yl)quinolin-6-yl)methylene)thiazolidine-2,4-dione (V-4). The title compound was prepared via HATU coupling as described in previous examples by reacting (Z)-5-((4-(6-(piperazin-1-yl)pyridin-3-yl)quinolin-6-yl)methylene)thiazolidine-2,4-dione (synthesized according to WO 2007136940A2) with acrylic acid. MS: m/z 472.0 (ES+).

In a similar fashion, using different boronic acid in preparing the intermediate above and/or using various acids in HATU coupling step, the following compounds were synthesized.

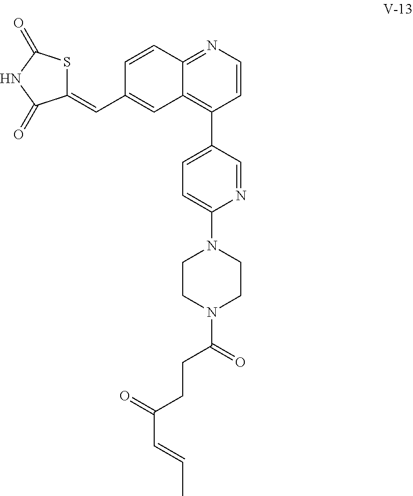

(Z)-5-((4-(6-(4-((E)-4-oxohept-5-enoyl)piperazin-1-yl)pyridin-3-yl)quinolin-6-yl)methylene)thiazolidine-2,4-dione (V-13). MS: m/z 542.7 (ES+).

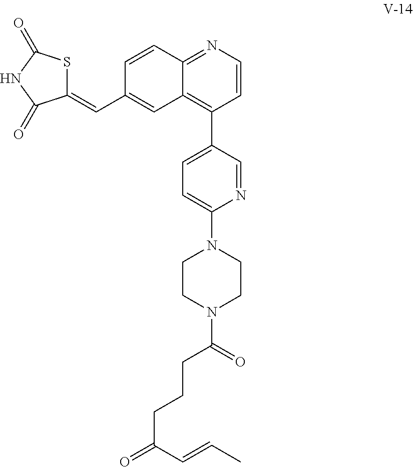

(Z)-5-((4-(6-(4-((E)-5-oxooct-6-enoyl)piperazin-1-yl)pyridin-3-yl)quinolin-6-yl)methylene)thiazolidine-2,4-dione (V-14). MS: m/z 556.2 (ES+).

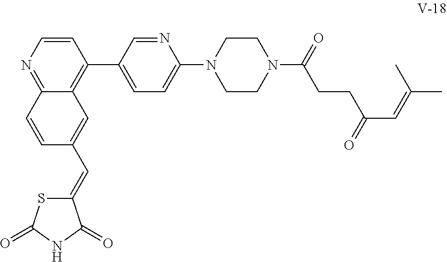

(Z)-5-((4-(6-(4-(6-methyl-4-oxohept-5-enoyl)piperazin-1-yl)pyridin-3-yl)quinolin-6-yl)methylene)thiazolidine-2,4-dione (V-18). MS: m/z 556.1 (ES+).

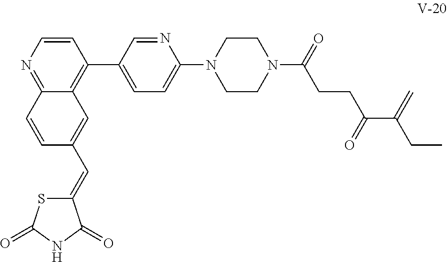

(Z)-5-((4-(6-(4-(5-methylene-4-oxoheptanoyl)piperazin-1-yl)pyridin-3-yl)quinolin-6-yl)methylene)thiazolidine-2,4-dione (V-20). MS: m/z 556.8 (ES+).

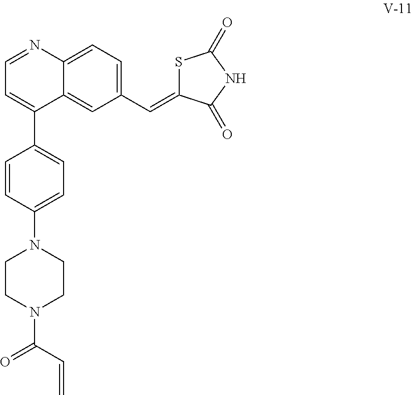

(Z)-5-((4-(4-(4-acryloylpiperazin-1-yl)phenyl)quinolin-6-yl)methylene)thiazolidine-2,4-dione (V-11). MS: m/z 471.7 (ES+).

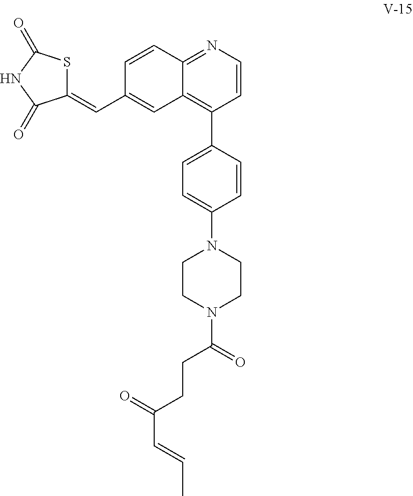

(Z)-5-((4-(4-(4-((E)-4-oxohept-5-enoyl)piperazin-1-yl)phenyl)quinolin-6-yl)methylene)thiazolidine-2,4-dione (V-15). MS: m/z 541.4 (ES+).

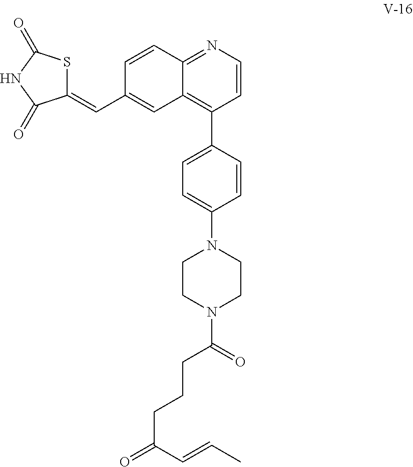

(Z)-5-((4-(4-(4-((E)-5-oxooct-6-enoyl)piperazin-1-yl)phenyl)quinolin-6-yl)methylene)thiazolidine-2,4-dione (V-16). Ms: m/z 555.3 (ES+).

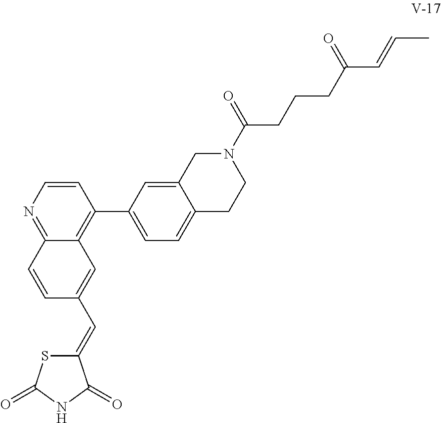

(Z)-5-((4-(2-((E)-5-oxooct-6-enoyl)-1,2,3,4-tetrahydroisoquinolin-7-yl)quinolin-6-yl)methylene)thiazolidine-2,4-dione (V-17). MS: m/z 526.6 (ES+).

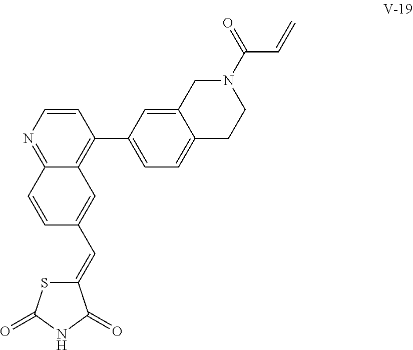

(Z)-5-((4-(2-acryloyl-1,2,3,4-tetrahydroisoquinolin-7-yl)quinolin-6-yl)methylene)thiazolidine-2,4-dione (V-19). MS: m/z 442.1 (ES+).

### Example 28

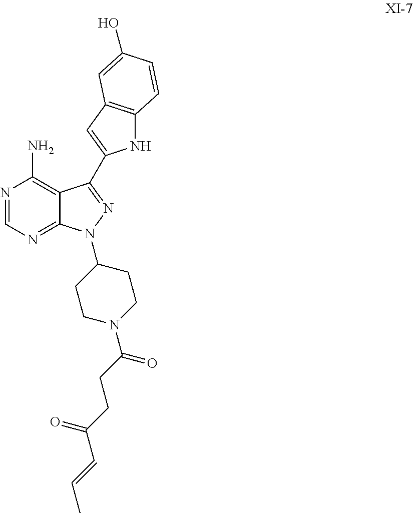

(E)-1-(4-(4-amino-3-(5-hydroxy-1H-indol-2-yl)-1H-pyrazolo[3,4-d]pyrimidin-1-yl)piperidin-1-yl)hept-5-ene-1,4-dione (XI-7). The title compound was prepared according to the following steps and intermediates described below.

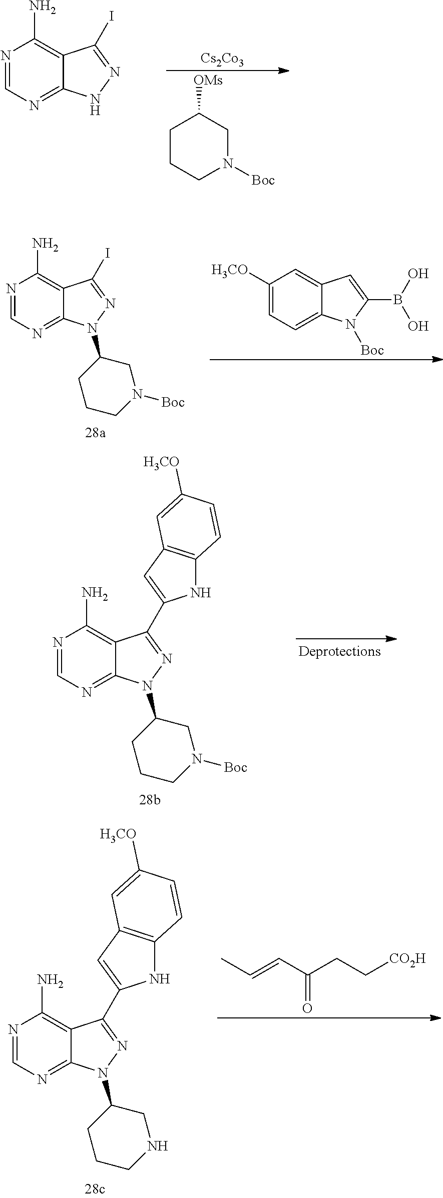

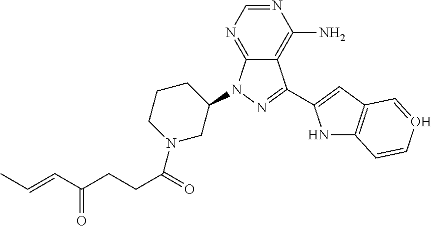

### Step 28a: (R)-tert-butyl 3-(4-amino-3-iodo-1H-pyrazolo[3,4-d]pyrimidin-1-yl)piperidine-1-carboxylate (Intermediate 28a)

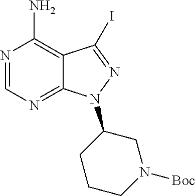

To a stirred solution of 3-iodo-1H-pyrazolo[3,4-d]pyrimidin-4-amine (500 mg, 1.9 mmol) in DMF (10 mL) was added cesium carbonate (1.56 g, 4.7 mmol) followed by (S)-tert-butyl 3-(methylsulfonyloxy)piperidine-1-carboxylate (535 mg, 1.9 mmol) at room temperature under N₂ atmosphere. The reaction mixture was heated to 80° C. and stirred further for 16 h at that temperature. After the completion of reaction (monitored by TLC), solvent was removed under reduced pressure, water was added and extracted with ethyl acetate (2×25 mL). The organic layer was separated, dried over Na₂SO₄ and solvent was removed under reduced pressure. The crude compound was purified by silica gel column chromatography [Methanol/DCM: 2/98] to afford Intermediate 28a (240 mg, 30%) as brown solid. TLC: 5% MeOH/DCM:ethylactate (1:1) (Rf: 0.3). ¹H-NMR (CDCl₃, 200 MHz): δ 8.38 (s, 1H), 6.02 (bs, 2H), 4.82-4.64 (1H), 4.31-4.02 (m, 2H), 3.44-3.20 (m, 1H), 2.95-2.65 (m, 1H), 2.25-2.08 (m, 2H), 1.95-1.58 (m, 2H), 1.42 (s, 9H). MS: m/z=445 (M⁺+1). Chiral HPLC purity (SAV-MA8002-56): 98.19% at 9.73 RT (0.1% TFA in hexane: ethanol/70:30, flow rate: 1 mL/min, Chiralpak, ADH, 250×4.6 mm, 5 um [SHCL061002].

### Step 28b: (R)-tert-butyl 3-(4-amino-3-(5-methoxy-1H-indol-2-yl)-1H-pyrazolo[3,4-d]pyrimidin-1-yl)piperidine-1-carboxylate (Intermediate 28b)

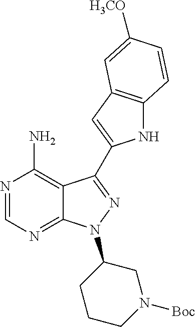

To a stirred solution of Intermediate 28a (100 mg, 0.33 mmol) in THF/H₂O (8 mL) was added 1-(tert-butoxycarbonyl)-5-methoxy-1H-indol-2-ylboronic acid (150 mg, 515 mmol), aqueous Na₂CO₃ (106 mg) (dissolved in minimum water) solution and Pd(TPP)₄ (10 mg). The reaction mixture was purged with argon for 1 h and further refluxed for 6 h. Progress of the reaction was monitored by TLC. The reaction mass was filtered through a pad of celite and concentrated the filtrate under vacuum. The crude compound was purified by column chromatography using 50% EtOAc/hexane to afford compound 3 (60 mg, 38.7%) as orange solid. TLC: 5% MeOH in EtOAc/DCM (1:1) (R_{f}: 0.5). ¹H-NMR (CDCl₃, 500 MHz): δ 8.83 (s, 1H), 8.38 (s, 1H), 7.34 (d, J=8.4 Hz, 2H), 7.08 (s, 1H), 6.94 (d, J=8 Hz, 1H), 6.82 (s, 1H), 5.91 (s, 2H), 4.97-4.91 (m, 1H), 4.32 (bs, 2H), 3.82 (s, 3H), 2.95 (bs, 2H), 2.62 (s, 1H), 2.5 (bs, 1H), 2.32-2.2 (m, 3H), 2.01 (d, 2H), 1.47 (s, 9H).

### Step 28c: (R)-2-(4-amino-1-(piperidin-3-yl)-1H-pyrazolo[3,4-d]pyrimidin-3-yl)-1H-indol-5-ol (Intermediate 28c)

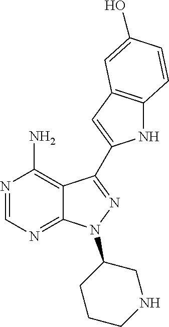

BBr₃ (4 mL) was added drop wise to a solution of Intermediate 28b (1.3 g, 2.8 mmol) in DCM (15 mL) at RT over a period of 15 minutes. The reaction mixture was stirred at RT for 16 h. Progress of the reaction was monitored by TLC. The volatiles were removed under reduced pressure, residue diluted with water (pH-7) and extracted with DCM (2×20 mL). The combined organic layers were dried over anhydrous Na₂SO₄ and concentrated in vacuo to afford compound 4 (800 mg, 80%) as orange solid. TLC: EtOAc (R_{f}: 0.1). MS: m/z=350 [M⁺+1]

### Step 28d: (E)-1-(4-(4-amino-3-(5-hydroxy-1H-indol-2-yl)-1H-pyrazolo[3,4-d]pyrimidin-1-yl)piperidin-1-yl)hept-5-ene-1,4-dione (XI-7)

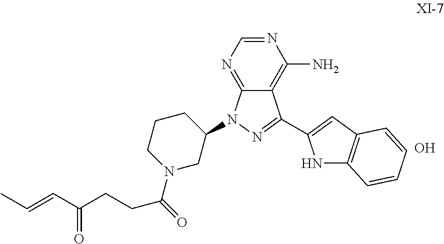

To a stirred solution of Intermediate 28c (300 mg, 0.86 mmol) in DCM (10 mL) was added (E)-4-oxohept-5-enoic acid (122 mg, 0.86 mmol), HATU (393 mg, 1.03 mmol) and DIPEA (333 mg, 2.5 mmol) at 0° C. Progress of the reaction was monitored by TLC immediately. After the reaction completion, the reaction mixture was quenched with ice cold water and extracted with DCM (3×20 mL). The combined organic layers were dried over anhydrous Na₂SO₄ and concentrated in vacuo. The crude compound was purified by column chromatography to afford XI-7 (25 mg, 10%) as off white solid. TLC: 10% MeOH/DCM (R_{f}: 0.3). ¹H-NMR (DMSO d₆, 500 MHz): δ 11.26 (s, 1H), 8.85 (d, J=8 Hz, 1H), 8.6 (s, 1H), 8.26 (d, J=8.2 Hz, 1H), 7.67 (d, J=7.2 Hz, 1H), 7.25 (m, 2H), 6.86 (m, 3H), 6.7 (m, 2H), 6.15-6.1 (m, 2H), 4.79 (bs, 1H), 4.6-4.52 (m, 2H), 4.28 (d, 1H), 4.13 (d, 1H), 4.02 (m, 1H), 3.62 (m, 1H), 3.08 (m, 2H), 2.78-2.36 (m, 7H), 1.95 (dd, 1H), 1.98 (bs, 2H), 1.8 (m, 6H), 1.7 (bs, 1H), 1.52 (bs, 1H). MS: m/z=474 [M⁺+1]

In a similar fashion, using different acid in the final step, the following compounds were synthesized.

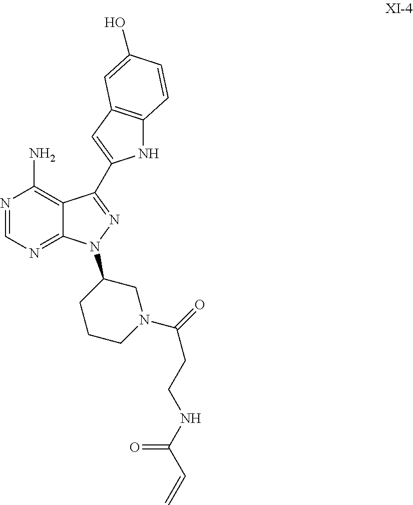

(R)—N-(3-(3-(4-amino-3-(5-hydroxy-1H-indol-2-yl)-1H-pyrazolo[3,4-d]pyrimidin-1-yl)piperidin-1-yl)-3-oxopropyl)acrylamide (XI-4). MS: m/z 475 (M+1).

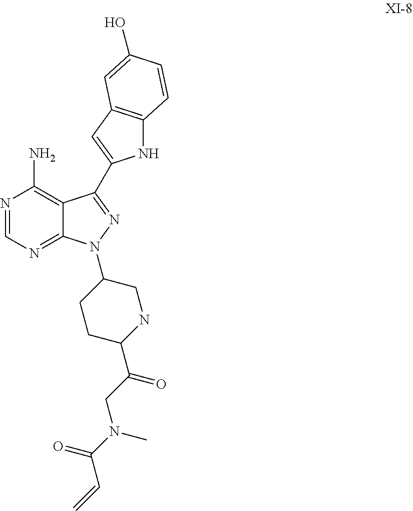

N-(2-(4-(4-amino-3-(5-hydroxy-1H-indol-2-yl)-1H-pyrazolo[3,4-d]pyrimidin-1-yl)piperidin-1-yl)-2-oxoethyl)-N-methylacrylamide (XI-8). MS: m/z 475 (M+1).

In a similar way, using tert-butyl 4-(methylsulfonyloxy)piperidine-1-carboxylate in step 28a, 4-amino-3-methoxyphenylboronic acid in step 28b, and appropriate acids in step 28c, the following compounds were prepared:

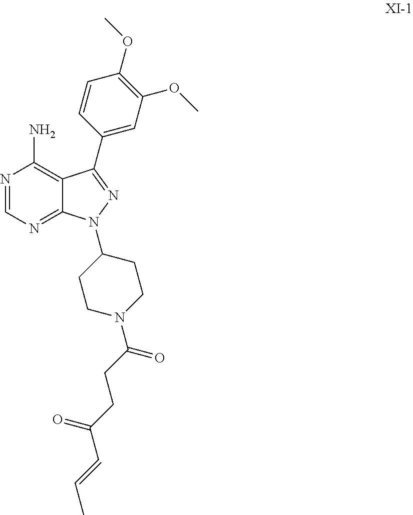

(E)-1-(4-(4-amino-3-(3,4-dimethoxyphenyl)-1H-pyrazolo[3,4-d]pyrimidin-1-yl)piperidin-1-yl)hept-5-ene-1,4-dione (XI-1). MS: m/z 479.2 (ES+).

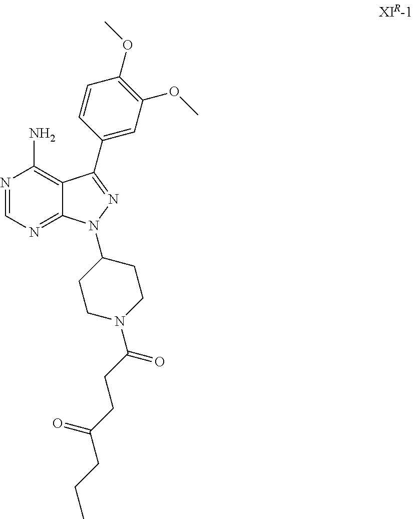

1-(4-(4-amino-3-(3,4-dimethoxyphenyl)-1H-pyrazolo[3,4-d]pyrimidin-1-yl)piperidin-1-yl)heptane-1,4-dione (XI^{R}-1). This compound was made by hydrogenation of XI-1. MS: m/z 481.2 (ES+).

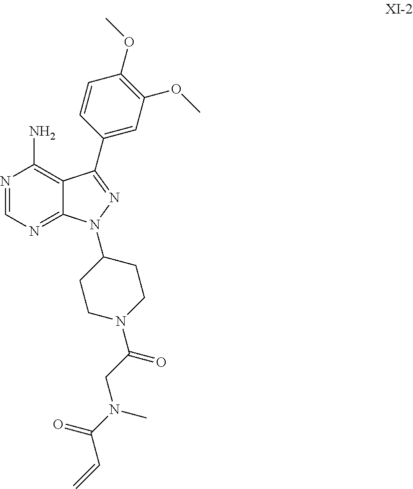

N-(2-(4-(4-amino-3-(3,4-dimethoxyphenyl)-1H-pyrazolo[3,4-d]pyrimidin-1-yl)piperidin-1-yl)-2-oxoethyl)-N-methylacrylamide (XI-2). MS: m/z 480.2 (ES+).

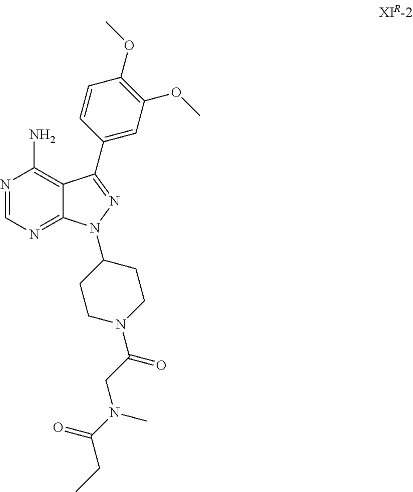

N-(2-(4-(4-amino-3-(3,4-dimethoxyphenyl)-1H-pyrazolo[3,4-d]pyrimidin-1-yl)piperidin-1-yl)-2-oxoethyl)-N-methylpropionamide (XI^{R}-2). This compound was made by hydrogenation on XI-2. MS: m/z 482.3 (ES+).

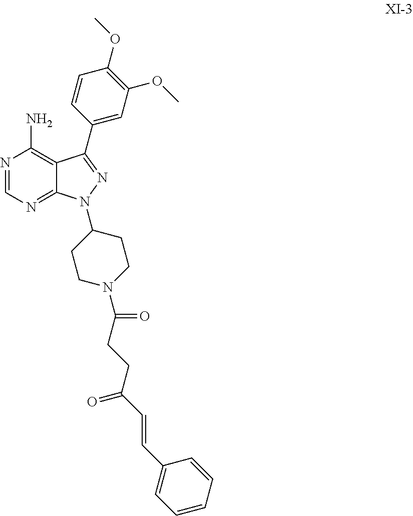

(E)-1-(4-(4-amino-3-(3,4-dimethoxyphenyl)-1H-pyrazolo[3,4-d]pyrimidin-1-yl)piperidin-1-yl)-6-phenylhex-5-ene-1,4-dione (XI-3). MS: m/z 541 (ES+).

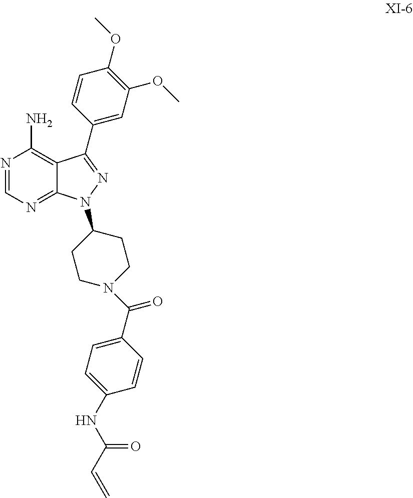

N-(4-(4-(4-amino-3-(3,4-dimethoxyphenyl)-1H-pyrazolo[3,4-d]pyrimidin-1-yl)piperidine-1-carbonyl)phenyl)acrylamide (XI-6). MS: m/z 527 (ES+).

### Example 29

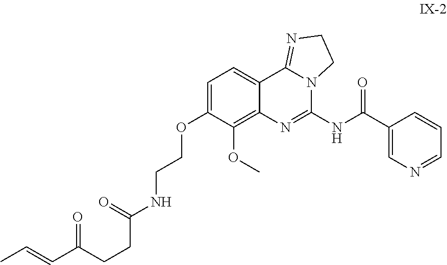

(E)-N-(7-methoxy-8-(2-(4-oxohept-5-enamido)ethoxy)-2,3-dihydroimidazo[1,2-c]quinazolin-5-yl)nicotinamide (IX-2). The title compound was prepared using the following intermediate described below.

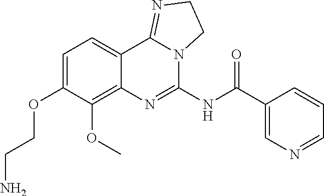

N-(8-(2-aminoethoxy)-7-methoxy-2,3-dihydroimidazo[1,2-c]quinazolin-5-yl)nicotinamide. The title intermediate was prepared according to patent WO2009091550A2.

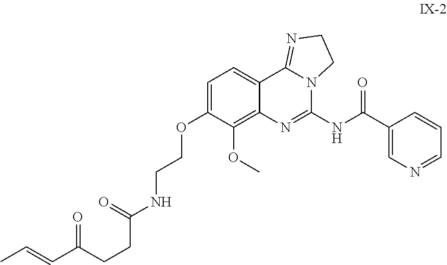

(E)-N-(7-methoxy-8-(2-(4-oxohept-5-enamido)ethoxy)-2,3-dihydroimidazo[1,2-c]quinazolin-5-yl)nicotinamide (IX-2). The title compound was prepared through the intermediate above using amide formation chemistry as described in previous examples. MS: m/z 505 (ES+).

In a similar fashion, using appropriate acids to react with the intermediate above, the following compounds were prepared:

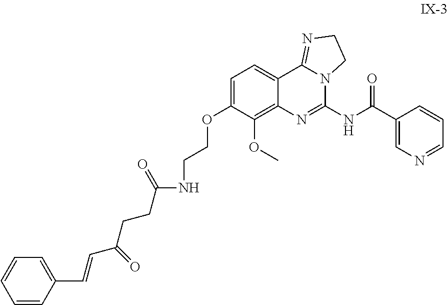

(E)-N-(7-methoxy-8-(2-(4-oxo-6-phenylhex-5-enamido)ethoxy)-2,3-dihydroimidazo[1,2-c]quinazolin-5-yl)nicotinamide (IX-3). MS: m/z 567 (ES+).

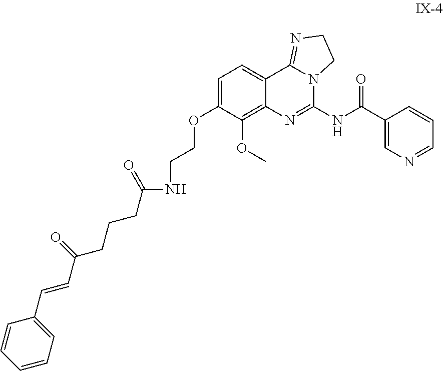

(E)-N-(7-methoxy-8-(2-(5-oxo-7-phenylhept-6-enamido)ethoxy)-2,3-dihydroimidazo[1,2-c]quinazolin-5-yl)nicotinamide (IX-4). MS: m/z 581 (ES+).

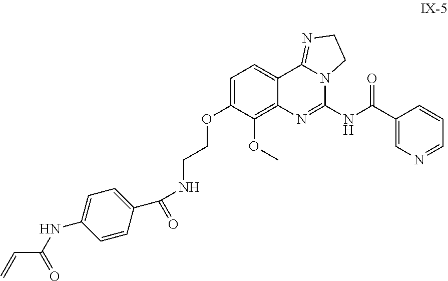

N-(8-(2-(4-acrylamidobenzamido)ethoxy)-7-methoxy-2,3-dihydroimidazo[1,2-c]quinazolin-5-yl)nicotinamide (IX-5). MS: m/z 554 (ES+).

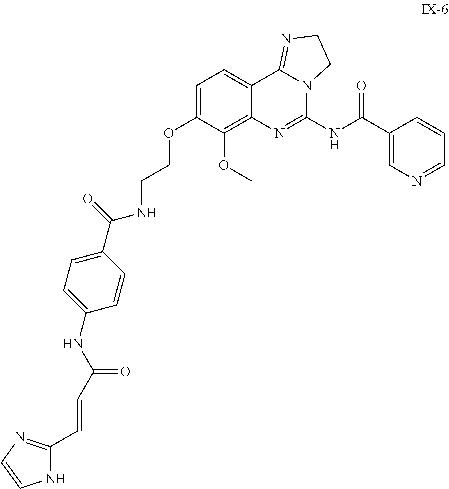

(E)-N-(8-(2-(4-(3-(1H-imidazol-2-yl)acrylamido)benzamido)ethoxy)-7-methoxy-2,3-dihydroimidazo[1,2-c]quinazolin-5-yl)nicotinamide (IX-6). MS: m/z 620.3 (ES+).

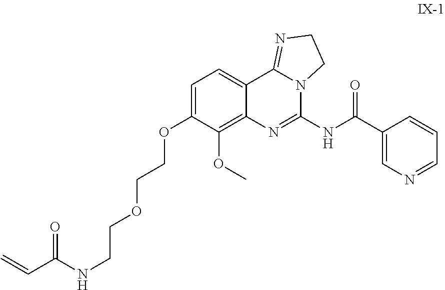

N-(8-(2-(2-acrylamidoethoxy)ethoxy)-7-methoxy-2,3-dihydroimidazo[1,2-c]quinazolin-5-yl)nicotinamide (IX-1). The title compound was prepared using acrylic acid to react with N-(8-(2-(2-aminoethoxy)ethoxy)-7-methoxy-2,3-dihydroimidazo[1,2-c]quinazolin-5-yl)nicotinamide, which synthesis was described in page 99 of patent WO2009091550A2. MS: m/z 479 (ES+).

### Example 30

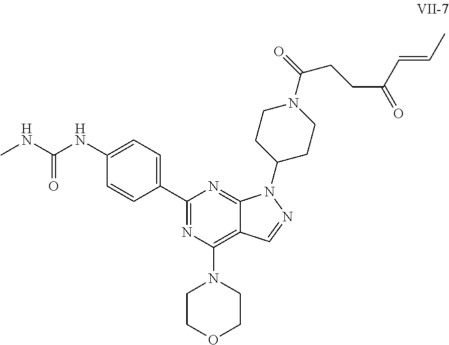

(E)-1-methyl-3-(4-(4-morpholino-1-(1-(4-oxohept-5-enoyl)piperidin-4-yl)-1H-pyrazolo[3,4-d]pyrimidin-6-yl)phenyl)urea (VII-7). The title compound was prepared through HATU coupling as described in previous examples, using (E)-4-oxohept-5-enoic acid and 1-methyl-3-(4-(4-morpholino-1-(piperidin-4-yl)-1H-pyrazolo[3,4-d]pyrimidin-6-yl)phenyl)urea, which was synthesized according to J. Med. Chem. 2009, 52 (16), 5013-5016. MS: m/z 560.8 (ES+).

In similar fashion, the following compounds were prepared using appropriate acids or alkyl halide to react with the same intermediate as for VII-7.

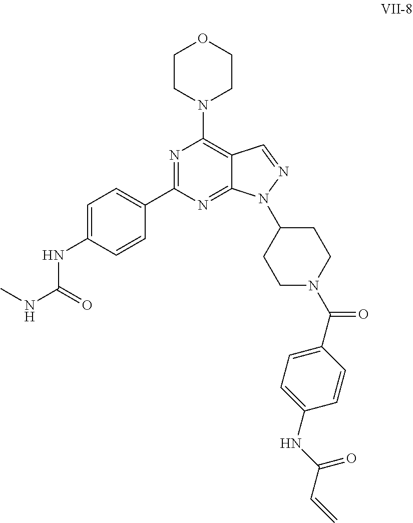

N-(4-(4-(6-(4-(3-methylureido)phenyl)-4-morpholino-1H-pyrazolo[3,4-d]pyrimidin-1-yl)piperidine-1-carbonyl)phenyl)acrylamide (VII-8). MS: m/z 609.7 (ES+).

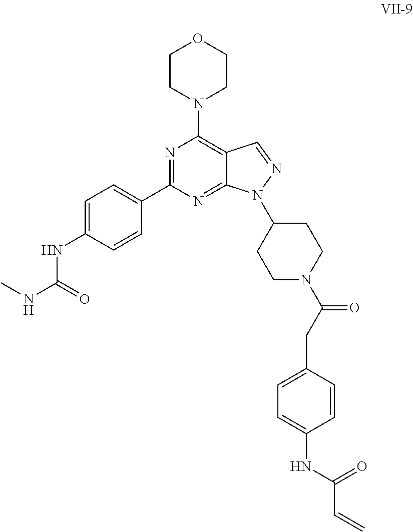

N-(4-(2-(4-(6-(4-(3-methylureido)phenyl)-4-morpholino-1H-pyrazolo[3,4-d]pyrimidin-1-yl)piperidin-1-yl)-2-oxoethyl)phenyl)acrylamide (VII-9). MS: m/z 623.7 (ES+).

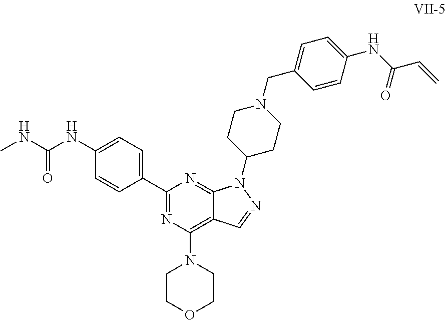

N-(4-((4-(6-(4-(3-methylureido)phenyl)-4-morpholino-1H-pyrazolo[3,4-d]pyrimidin-1-yl)piperidin-1-yl)methyl)phenyl)acrylamide (VII-5). MS: m/z 595.8 (ES+).

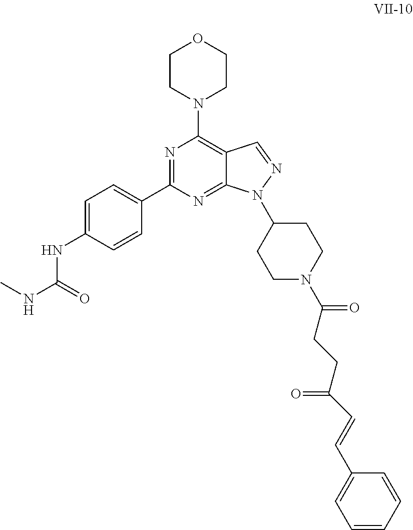

(E)-1-methyl-3-(4-(4-morpholino-1-(1-(4-oxo-6-phenylhex-5-enoyl)piperidin-4-yl)-1H-pyrazolo[3,4-d]pyrimidin-6-yl)phenyl)urea (VII-10). MS: m/z 622.7 (ES+).

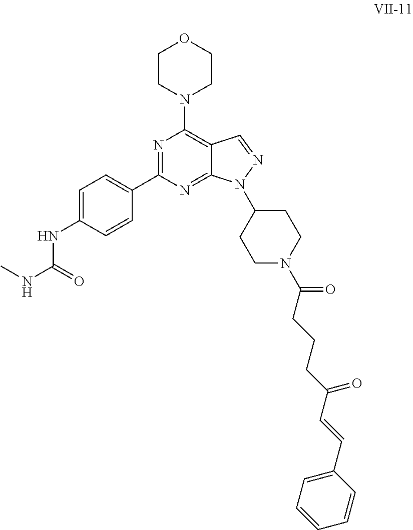

(E)-1-methyl-3-(4-(4-morpholino-1-(1-(5-oxo-7-phenylhept-6-enoyl)piperidin-4-yl)-1H-pyrazolo[3,4-d]pyrimidin-6-yl)phenyl)urea (VII-11). MS: m/z 636.7 (ES+).

Following similar chemistry described in J. Med. Chem. 2009, 52 (16), 5013-5016, using 2-aminopyrimidine 5-boronic acid, the following two compounds were synthesized.

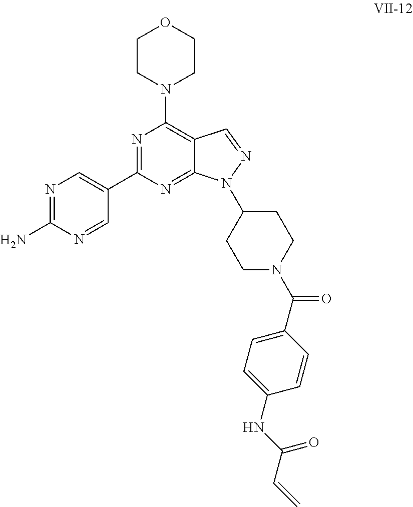

N-(4-(4-(6-(2-aminopyrimidin-5-yl)-4-morpholino-1H-pyrazolo[3,4-d]pyrimidin-1-yl)piperidine-1-carbonyl)phenyl)acrylamide (VII-12). MS: m/z 555.2 (ES+).

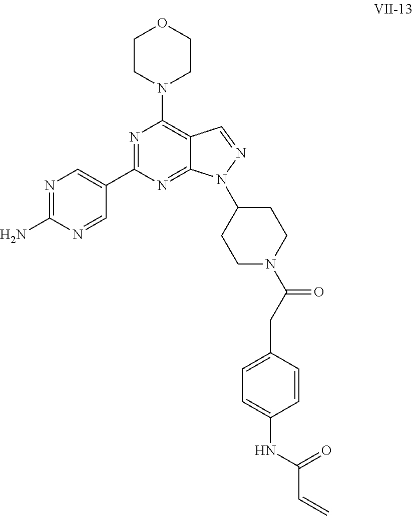

N-(4-(2-(4-(6-(2-aminopyrimidin-5-yl)-4-morpholino-1H-pyrazolo[3,4-d]pyrimidin-1-yl)piperidin-1-yl)-2-oxoethyl)phenyl)acrylamide (VII-13). MS: m/z 569.3 (ES+).

### Example 31

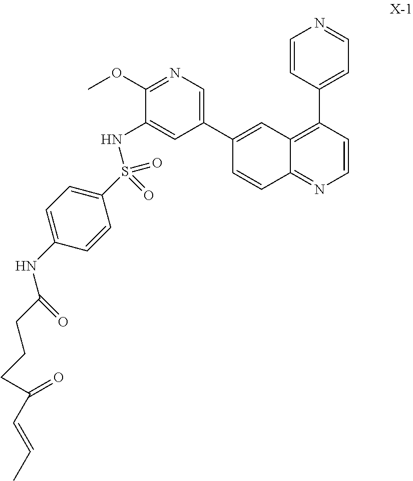

(E)-N-(4-(N-(2-methoxy-5-(4-(pyridin-4-yl)quinolin-6-yl)pyridin-3-yl)sulfamoyl)phenyl)-5-oxooct-6-enamide (X-1). The title compound was prepared via HATU coupling reaction by reacting (E)-5-oxooct-6-enoic acid with appropriate aniline intermediate (synthesized according to the published paper ACS Medicinal Chemistry Letters 2010, 1(1), 39-43.). MS: m/z 622.2 (ES+).

### Example 32

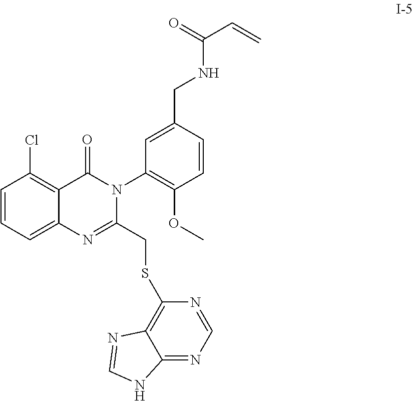

N-(3-(2-((9H-purin-6-ylthio)methyl)-5-chloro-4-oxoquinazolin-3(4H)-yl)-4-methoxybenzyl)acrylamide (I-5). The title compound was prepared via HATU coupling by reacting acrylic acid and 2-((9H-purin-6-ylthio)methyl)-3-(5-(aminomethyl)-2-methoxyphenyl)-5-chloroquinazolin-4(3H)-one, which was synthesized according to WO 01/81346. ¹H NMR: (DMSO, 400 MHz): δ 3.567 (s, 3H), 4.177 (s, 2H), 4.373 (d, 2H), 5.566 (1H, d), 6.068 (1H, D), 6.233 (t, 1H), 7.071-7.775 (m, 8H), 13.55 (s, 1H). MS: m/z 534.1 (M+1).

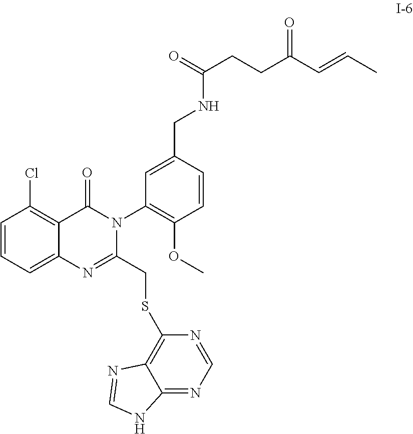

(E)-N-(3-(2-((9H-purin-6-ylthio)methyl)-5-chloro-4-oxoquinazolin-3(4H)-yl)-4-methoxybenzyl)-4-oxohept-5-enamide (I-6). In a similar fashion, using (E)-4-oxohept-5-enoic acid instead of acrylic acid, I-6 was prepared. ¹H NMR: (DMSO, 400 MHz): δ 2.309 (d, 3H), 2.808 (t, 2H), 3.684 (t, 2H), 3.728 (s, 3H), 4.244 (dd, 2H), 4.420 (d, 2H), 6.662-8.467 (m, 8H), 9.048 (s, 1H). MS: m/z 604.1 (M+1).

### Example 33

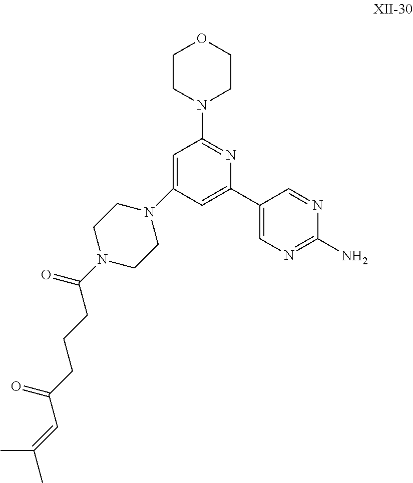

1-(4-(2-(2-aminopyrimidin-5-yl)-6-morpholinopyridin-4-yl)piperazin-1-yl)-7-methyloct-6-ene-1,5-dione (XII-30). The title compound was synthesized through the following intermediates and steps as described below.

### tert-butyl 4-(2-chloro-6-morpholinopyridin-4-yl)piperazine-1-carboxylate (Intermediate 33a)

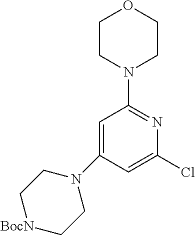

### Method A

A reaction mixture of 4-(6-chloro-4-iodopyridin-2-yl)morpholine (Intermediate 22a, 97 mg, 0.3 mmol), N-Boc-piperazine (60 mg, 0.32 mmol), and 200 uL of DIPEA in 1 mL of DMA was heated at 150° C. in CEM-microwave for 30 min. The reaction mixture was suspended in EtOAc, washed with water, and dried over Na₂SO₄. After filtration and concentration, the residue was purified by column chromatography on silica gel, with heptanes/EtOAc (v/v 3/2) as eluent, giving 15 mg of desired product. Most of the starting material was recovered. MS: m/z 383.2 (ES+).

### Method B

A mixture of 4-(6-chloro-4-iodopyridin-2-yl)morpholine (Intermediate 22a, 324 mg, 1.0 mmol), N-Boc-piperazine (192 mg, 1.05 mmol), 150 mg of sodium t-butoxide (1.5 equiv.), tris(dibenzylideneacetone)dipalladium (27.2 mg, 3% mol) in 10 mL of dioxane was purged with nitrogen for 15 min, followed by addition of 120 uL of 0.5 M tributylphosphine solution in toluene. The resulting mixture was stirred at room temperature over weekend. The solvent was then removed under reduced pressure, and the residue was subject to regular workup with EtOAc-water, and dried over Na₂SO₄. After filtration and concentration, the crude product was purified by column chromatography on silica gel, with heptanes/EtOAc (v/v 3/2) as eluent, giving 275 mg of desired product as slight yellow solid. MS: m/z 383.2 (ES+).

### 1-(4-(2-chloro-6-morpholinopyridin-4-yl)piperazin-1-yl)-7-methyloct-6-ene-1,5-dione (Intermediate 33b)

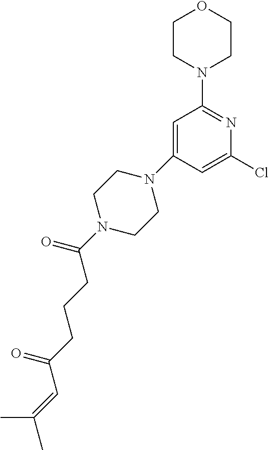

Intermediate 33a (15 mg) was treated with 0.6 mL of trifluoroacetic acid in 1 mL of dichloromethane. After 30 min, the excess amount of TFA and DCM were evaporated and the residue was dried in vacuum. The de-Boc intermediate was then reacted with 7-methyl-5-oxooct-6-enoic acid using HATU coupling as described in the previous examples, giving 9 mg of Intermediate 33b as yellow semi-solid. MS: m/z 435.1 (ES+).

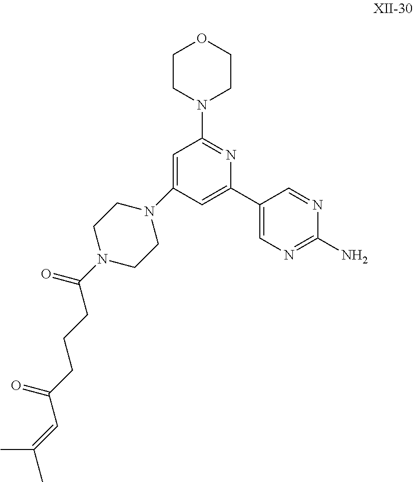

1-(4-(2-(2-aminopyrimidin-5-yl)-6-morpholinopyridin-4-yl)piperazin-1-yl)-7-methyloct-6-ene-1,5-dione (XII-30). Intermediate 33b underwent Suzuki coupling with 2-amino-5-boronic acid under the condition as described in the previous examples, giving XII-30. MS: m/z 494.2 (ES+).

In a similar fashion, using different cyclic amines and/or various acids in final HATU coupling, or alkylating reagent to react with amine in final step, the following compounds were synthesized.

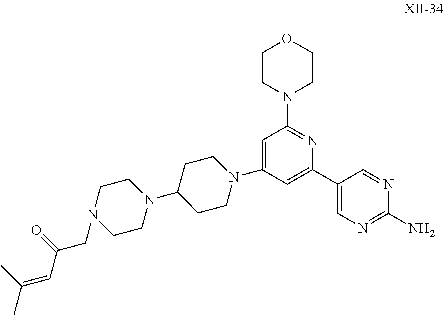

1-(4-(1-(2-(2-aminopyrimidin-5-yl)-6-morpholinopyridin-4-yl)piperidin-4-yl)piperazin-1-yl)-4-methylpent-3-en-2-one (XII-34). MS: m/z 521.3 (ES+).

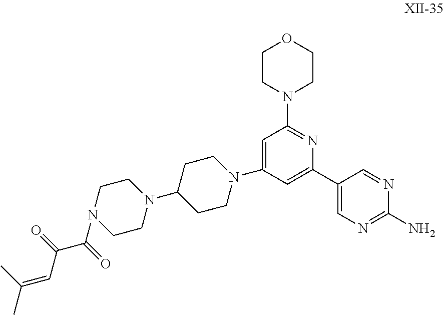

1-(4-(1-(2-(2-aminopyrimidin-5-yl)-6-morpholinopyridin-4-yl)piperidin-4-yl)piperazin-1-yl)-4-methylpent-3-ene-1,2-dione (XII-35). MS: m/z 535.2 (ES+).

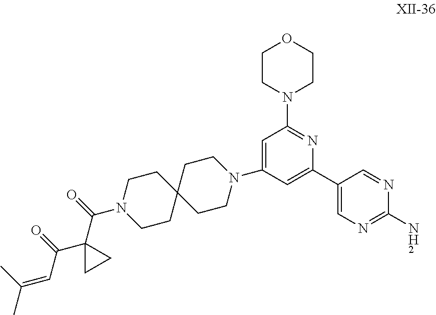

1-(1-(9-(2-(2-aminopyrimidin-5-yl)-6-morpholinopyridin-4-yl)-3,9-diazaspiro[5.5]undecane-3-carbonyl)cyclopropyl)-3-methylbut-2-en-1-one (XII-36). MS: m/z 560.2 (ES+).

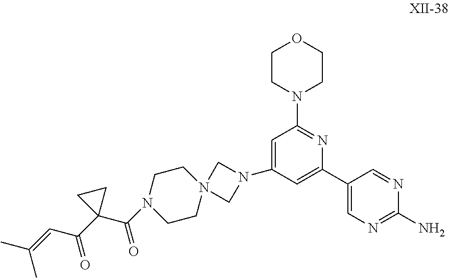

1-(1-(2-(2-(2-aminopyrimidin-5-yl)-6-morpholinopyridin-4-yl)-2,7-diazaspiro[3.5]nonane-7-carbonyl)cyclopropyl)-3-methylbut-2-en-1-one (XII-38). MS: m/z 532.2 (ES+).

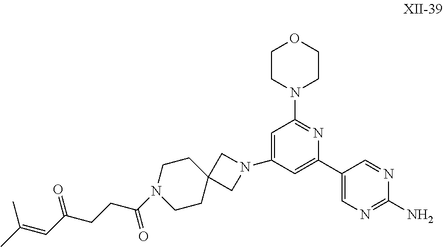

1-(2-(2-(2-aminopyrimidin-5-yl)-6-morpholinopyridin-4-yl)-2,7-diazaspiro[3.5]nonan-7-yl)-6-methylhept-5-ene-1,4-dione (XII-39). MS: m/z 520.2 (ES+).

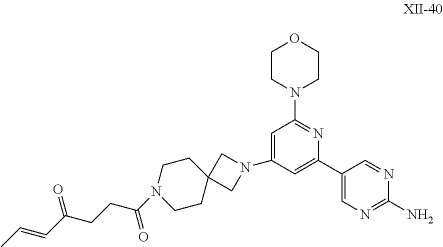

(E)-1-(2-(2-(2-aminopyrimidin-5-yl)-6-morpholinopyridin-4-yl)-2,7-diazaspiro[3.5]nonan-7-yl)hept-5-ene-1,4-dione (XII-40). MS: m/z 506.2 (ES+).

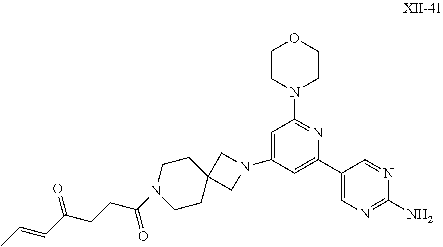

1-(2-(2-(2-aminopyrimidin-5-yl)-6-morpholinopyridin-4-yl)-2,7-diazaspiro[3.5]nonan-7-yl)-7-methyloct-6-ene-1,5-dione (XII-41). MS: m/z 534.3 (ES+).

In a similar fashion, using different cyclic amines and/or various acids in final HATU coupling, or alkylating reagent to react with amine in final step, the following compounds were synthesized having used Method B (described above) in the synthesis of intermediate 33a).

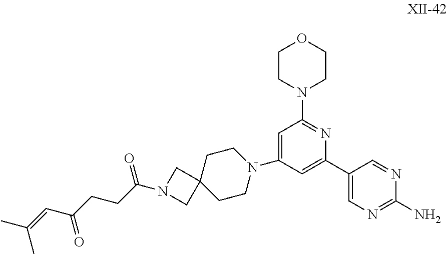

1-(7-(2-(2-aminopyrimidin-5-yl)-6-morpholinopyridin-4-yl)-2,7-diazaspiro[3.5]nonan-2-yl)-6-methylhept-5-ene-1,4-dione (XII-42). MS: m/z 520.2 (ES+).

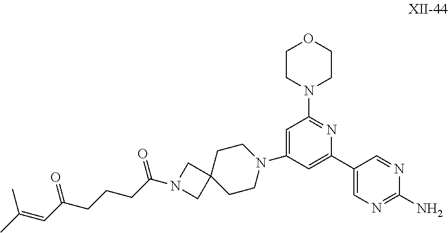

1-(7-(2-(2-aminopyrimidin-5-yl)-6-morpholinopyridin-4-yl)-2,7-diazaspiro[3.5]nonan-2-yl)-7-methyloct-6-ene-1,5-dione (XII-44). MS: m/z 534.2 (ES+).

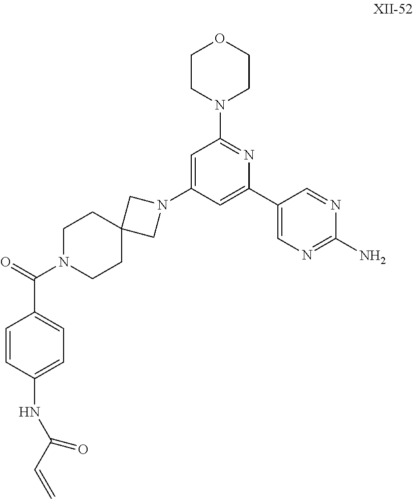

N-(4-(2-(2-(2-aminopyrimidin-5-yl)-6-morpholinopyridin-4-yl)-2,7-diazaspiro[3.5]nonane-7-carbonyl)phenyl)acrylamide (XII-52). MS: m/z 555.2 (ES+).

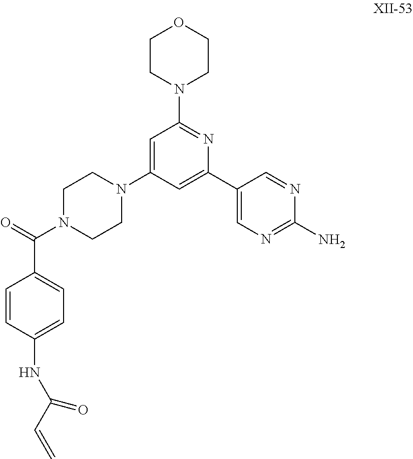

N-(4-(4-(2-(2-aminopyrimidin-5-yl)-6-morpholinopyridin-4-yl)piperazine-1-carbonyl)phenyl)acrylamide (XII-53). MS: m/z 515.2 (ES+).

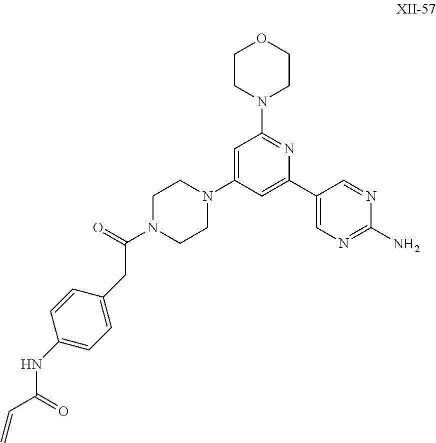

N-(4-(2-(4-(2-(2-aminopyrimidin-5-yl)-6-morpholinopyridin-4-yl)piperazin-1-yl)-2-oxoethyl)phenyl)acrylamide (XII-57). MS: m/z 529.2 (ES+).

### Biological Examples

Described below are assays used to measure the biological activity of provided compounds as inhibitors of PI3 kinases.

### Example 34

Compounds of the present invention are assayed as inhibitors of PI3 kinases using the following general protocol.

### Homogeneous Time Resolved Fluorescence (HTRF) Assay Protocol for Potency Assessment Against the Active Forms of PI3Kα, PI3Kβ, and PI3Kγ

The protocol below describes an end-point, competition-binding HTRF assay used to measure inherent potency of test compounds against active PI3Kα (p110α/p85α), PI3Kβ (p110β/p85α), and PI3Kγ (p120γ) enzymes. The mechanics of the assay platform are best described by the vendor (Millipore, Billerica, Mass.) on their website at the following URL: www.millipore.com/coa/tech1/74jt4z.

Briefly, Stop solution (Stop A, #33-007 and Stop B, #33-009; 3:1 ratio) and Detection Mix (from DMC, #33-015 with DMA, #33-011 and DMB, #33-013; 18:1:1 ratio) were prepared as recommended by the manufacturer about 2 hrs prior to use. Additionally, 1× reaction buffer (from 4× buffer stock# 33-003), 1.4× stocks of PI3Kα, PI3Kβ, and PI3Kγ enzymes from BPS Bioscience (San Diego, Calif.) or Millipore (Billerica, Mass.) with di-C₈-PIP₂ lipid substrate (#33-005), and a 4×ATP solution (#A7699 Sigma/Aldrich; St. Louis, Mo.) were prepared in 1× reaction buffer. 15 μL of PI3K enzymes and lipid substrate mix were pre-incubated in a Corning (#3573) 384-well, black, non-treated microtiter plate (Corning, N.Y.) for 30 min at 25° C. with a 0.5 μL volume of 50% DMSO and serially diluted compounds prepared in 50% DMSO. Lipid kinase reactions were started with the addition of 5 μL of ATP solution, mixed for 15 sec on a rotary plate shaker and incubated for 30-60 minutes at 25° C. Next, reactions were stopped with a 5 μL addition of Stop solution immediately followed by a 5 μL volume of Detection Mix. Stopped reactions were equilibrated for 1 and 18 hrs at room temperature and read in a Synergy⁴ plate reader from BioTek (Winooski, Vt.) at λₑₓ330-80/λₑₘ620-35 and λₑₘ665-7.5. At the conclusion of each assay, the HTRF ratio from fluorescence emission values for each well was calculated and % Inhibition determined from averaged controls wells (+/−PI3K enzyme). % Inhibition values for each compound were then plotted against inhibitor concentration to estimate IC₅₀ from log [Inhibitor] vs Response, Variable Slope model in GraphPad Prism from GraphPad Software (San Diego, Calif.).

[Reagent] used in optimized protocol:
[p110α/p85α]=0.5-1.5 nM, [ATP]=50 μM, [di-C₈—PIP₂]=10 μM
[p110β/p85α]=0.75 nM, [ATP]=50 μM, [di-C₈—PIP₂]=10 μM
[p120γ]=2-2.5 nM, [ATP]=50 μM, [di-C₈—PIP₂]=10 μM
(ATP K_{Mapp} for both enzymes was estimated to be 40-70 μM)

Reference Inhibitor IC₅₀s estimated for p110α/p85α-p120γ enzymes:

LY294002=2-5 μM (n=6; published IC₅₀=0.7 to 3 μM)
Wortmannin=3-13 nM (n=5; published IC₅₀=2 to 9 nM)

Reference Inhibitor IC₅₀s estimated for p110β/p85α enzyme:
LY294002=>1 μM (n=6; published IC₅₀=>1 μM)
PIK-75=248 nM (n=10; published IC₅₀=343 nM)

### Example 35

Table 20 shows the activity of selected compounds of this invention in the PI3Kα, PI3Kβ, and PI3Kγ HTRF assays. Compounds having an activity designated as “A” provided an IC₅₀≦10 nM; compounds having an activity designated as “B” provided an IC₅₀ of 10-100 nM; compounds having an activity designated as “C” provided an IC₅₀ of 100-1000 nM; and compounds having an activity designated as “D” provided an IC₅₀ of ≧1000 nM. “-” indicates that the value was not determined.

TABLE 20
PI3K Inhibition Data

| Compound # | PI3Kα Inhibition | PI3Kγ Inhibition | PI3Kβ Inhibition |
|---|---|---|---|
| I-5 | D | — | — |
| I-6 | D | — | — |
| GDC-941 | B | — | — |
| II-a-1 | C | C | — |
| II-a-2 | B | — | — |
| II-a-3 | C | — | D |
| II-a-6 | C | D | — |
| II-a-13 | B | D | — |
| II-a-14 | B | D | — |
| II-a-16 | A | D | — |
| II^{R}-a-16 | C | D | — |
| II-a-17 | D | D | — |
| II-a-19 | B | — | — |
| II-a-20 | C | — | — |
| II-a-21 | D | D | — |
| II-a-22 | B | D | — |
| II-a-23 | C | D | — |
| II-a-24 | B | D | — |
| II-a-25 | B | C | — |
| II-a-26 | B | C | — |
| II-a-27 | C | D | — |
| II-a-28 | C | C | — |
| II-a-29 | B | C | — |
| II-a-31 | C | D | — |
| II-a-32 | C | D | — |
| II-a-33 | B | D | — |
| II-a-34 | C | D | — |
| II-a-35 | C | C | — |
| II-a-36 | A | D | D |
| II^{R}-a-36 | D | — | — |
| II-a-37 | A | D | — |
| II-a-38 | B | D | — |
| II-a-39 | B | D | — |
| II-a-40 | B | C | — |
| II-a-41 | D | D | — |
| II-a-42 | D | D | — |
| II-a-43 | B | D | D |
| II-a-44 | D | D | — |
| II-a-45 | A | C | — |
| II-a-46 | B | C | — |
| II-a-47 | B | D | — |
| II-a-48 | A | D | — |
| II-a-49 | B | D | — |
| II-a-50 | A | D | — |
| II-a-51 | C | D | — |
| II-a-52 | C | D | — |
| II-a-53 | A | D | — |
| II-a-54 | B | D | — |
| II-a-55 | B | D | D |
| II-a-56 | C | D | — |
| II-a-57 | C | D | — |
| II-a-58 | B | D | — |
| II-a-59 | D | D | — |
| II-a-60 | B | — | — |
| II-a-61 | A | — | — |
| II-a-62 | B | — | — |
| II-a-63 | A | — | — |
| II-a-64 | A | — | — |
| II^{R}-a-64 | C | — | — |
| II-a-65 | A | — | — |
| II-a-66 | B | — | — |
| II-a-67 | A | — | — |
| II-a-68 | A | — | — |
| II-a-69 | B | — | — |
| II-a-70 | B | — | — |
| II-a-78 | C | — | — |
| II-a-79 | A | — | — |
| II-a-80 | A | — | — |
| II-a-81 | B | — | — |
| II^{R}-a-81 | C | — | — |
| II-a-86 | B | — | — |
| II-a-89 | A | — | — |
| II-a-95 | D | — | — |
| II-a-96 | C | — | — |
| II-a-97 | C | — | — |
| II-a-98 | C | — | — |
| II-a-99 | C | — | — |
| II-a-100 | C | — | — |
| II-a-101 | C | — | — |
| II-a-102 | A | — | — |
| II-a-103 | A | — | — |
| II-a-104 | A | — | — |
| II-a-105 | A | — | — |
| II-a-106 | B | — | — |
| II-a-107 | C | — | — |
| II-a-108 | A | — | — |
| II-a-109 | A | — | — |
| II-a-110 | C | — | — |
| II-a-111 | B | — | — |
| II-a-112 | B | — | C |
| II-a-113 | D | — | — |
| II-a-114 | C | — | — |
| II-a-115 | B | — | — |
| II-a-116 | B | — | D |
| II-a-117 | C | — | — |
| II-a-118 | C | — | — |
| II-a-119 | C | — | — |
| II-a-120 | C | — | — |
| II-a-121 | C | — | — |
| II-a-122 | B | — | — |
| II-a-123 | A | — | — |
| II-a-124 | C | — | — |
| II-a-125 | C | — | — |
| II-a-126 | C | — | — |
| II-a-127 | C | — | — |
| II-a-128 | C | — | — |
| II-a-129 | C | — | — |
| II-a-130 | C | — | — |
| II-a-131 | C | — | — |
| II-a-132 | C | — | — |
| II-a-133 | C | — | — |
| II-a-134 | C | — | — |
| II-a-135 | C | — | — |
| II-a-136 | C | — | — |
| II-a-137 | B | — | — |
| II-a-138 | B | — | — |
| II-a-139 | B | — | — |
| II-a-140 | B | — | — |
| II-a-141 | B | — | — |
| II-a-142 | A | — | — |
| II-a-143 | C | — | — |
| II-a-144 | C | — | C |
| II-a-145 | C | — | — |
| II-a-146 | D | — | — |
| II-a-147 | C | — | — |
| II-a-148 | C | — | D |
| II^{R}-a-148 | C | D | D |
| II-a-149 | C | — | — |
| II-a-150 | B | — | — |
| II-a-151 | D | — | — |
| II-a-152 | C | — | — |
| II-a-153 | C | — | — |
| II-a-154 | B | — | — |
| II-a-155 | B | — | — |
| II-a-156 | B | — | — |
| II-a-157 | C | — | — |
| II-a-158 | B | — | — |
| II-a-159 | B | — | — |
| II-a-160 | C | — | D |
| II-a-161 | C | — | — |
| II-a-163 | D | — | — |
| II-a-164 | B | — | C |
| II-a-165 | B | — | — |
| II-a-166 | A | — | — |
| II-a-167 | C | — | — |
| II-a-168 | C | — | — |
| II-a-169 | B | C | D |
| II-a-170 | C | — | — |
| II-a-171 | A | — | — |
| II-a-172 | C | — | — |
| II-a-173 | C | — | — |
| II-a-174 | B | — | — |
| II-a-175 | B | — | — |
| II-a-176 | B | — | — |
| II-a-177 | C | — | — |
| II-g-1 | C | — | — |
| II-g-2 | C | C | — |
| II-g-3 | C | C | — |
| II-g-4 | D | — | — |
| II-g-5 | D | — | — |
| II-g-6 | C | — | — |
| II-g-7 | C | — | — |
| II-g-8 | C | — | — |
| V-2 | C | D | — |
| V-3 | C | D | — |
| V-4 | B | — | — |
| V-11 | B | — | — |
| V-13 | A | — | — |
| V-14 | A | — | — |
| V-15 | B | — | — |
| V-16 | A | — | — |
| V-17 | B | — | — |
| V-18 | A | — | — |
| V-19 | B | — | — |
| V-20 | A | — | — |
| VI-1 | D | C | — |
| VI-24 | D | — | — |
| VI-25 | D | — | — |
| VII-5 | C | — | — |
| VII-7 | C | — | — |
| VII-8 | C | — | — |
| VII-9 | C | — | — |
| VII-10 | C | — | — |
| VII-11 | C | — | — |
| VII-12 | C | — | — |
| VII-13 | C | — | — |
| IX-1 | B | — | — |
| IX-2 | B | — | — |
| IX-3 | B | — | — |
| IX-4 | C | — | — |
| IX-5 | B | — | — |
| IX-6 | B | — | — |
| X-1 | C | — | — |
| XI-ref | D | — | — |
| XI-1 | D | — | — |
| XI^{R}-1 | D | — | — |
| XI-2 | D | — | — |
| XI^{R}-2 | D | — | — |
| XI-3 | C | — | — |
| XI-4 | D | — | — |
| XI-5 | D | — | — |
| XI-6 | D | — | — |
| XI-7 | D | — | — |
| XI-8 | D | — | — |
| XII-1 | C | — | — |
| XII-2 | B | — | — |
| XII-3 | B | — | — |
| XII-4 | B | — | — |
| XII-5 | C | — | — |
| XII-6 | C | — | — |
| XII-7 | D | — | — |
| XII-8 | D | — | — |
| XII-9 | D | — | — |
| XII-10 | C | — | — |
| XII-11 | D | — | — |
| XII-12 | D | — | — |
| XII-13 | D | — | — |
| XII-14 | D | — | — |
| XII-15 | C | — | — |
| XII-16 | C | — | — |
| XII-17 | D | — | — |
| XII-18 | D | — | — |
| XII-19 | D | — | — |
| XII-20 | C | — | — |
| XII-21 | D | — | — |
| XII-22 | A | — | — |
| XII-23 | C | — | — |
| XII-24 | B | — | — |
| XII-25 | B | — | — |
| XII-26 | B | — | — |
| XII-27 | B | — | — |
| XII-28 | C | — | — |
| XII-29 | C | — | — |
| XII-30 | C | — | — |
| XII-31 | C | — | — |
| XII-32 | C | — | — |
| XII-33 | C | — | — |
| XII-34 | C | — | — |
| XII-35 | C | — | — |
| XII-36 | C | — | — |
| XII-37 | B | — | — |
| XII-38 | C | — | — |
| XII-39 | B | — | — |
| XII-40 | C | — | — |
| XII-41 | D | — | — |
| XII-42 | D | — | — |
| XII-44 | D | — | — |
| XII-46 | C | — | — |
| XII-47 | C | — | — |
| XII-48 | C | — | — |
| XII-49 | B | — | — |
| XII-50 | C | — | — |
| XII-51 | B | — | — |
| XII-52 | C | — | — |
| XII-53 | C | — | — |
| XII-54 | C | — | — |
| XIV-a-2 | D | D | — |

### Example 36

### PI3K HCT116 Cellular Assay

Selected compounds were assayed in HCT116 colon cancer cells. HCT116 cells were plated overnight and then incubated for 1 hour with varying concentrations of inhibitors (5, 2, 0.5, 0.1 and 0.02 μM). Cells were then washed with PBS, lysed and the protein lysates were then recovered and analyzed by Western blot.

Table 21 shows the dose response of selected compounds of this invention in the PI3K HCT116 cellular inhibition assay. Compounds having an activity designated as “A” provided an EC₅₀≦20 nM; compounds having an activity designated as “B” provided an EC₅₀ of 20-100 nM; compounds having an activity designated as “C” provided an EC₅₀ of 100-500 nM; compounds having an activity designated as “D” provided an EC₅₀ of 500-2000 nM; compounds having an activity designated as “E” provided an EC₅₀ of 2000-5000 nM; and compounds having an activity designated as “F” provided an EC₅₀ of ≧5000 nM.

TABLE 21
PI3K HCT116 Cellular Inhibition Data

| Compound # | PI3K Inhibition |
|---|---|
| GDC-941 | C |
| II-a-6 | E |
| II-a-16 | C |
| II-a-25 | B |
| II-a-26 | B |
| II-a-28 | B |
| II-a-29 | C |
| II-a-33 | B |
| II-a-35 | C |
| II-a-36 | A |
| II-a-37 | B |
| II-a-43 | A |
| II-a-45 | C |
| II-a-46 | C |
| II-a-47 | C |
| II-a-48 | B |
| II-a-49 | A |
| II-a-50 | A |
| II-a-53 | B |
| II-a-55 | B |
| GSK-615 | A |
| V-3 | D |

### Example 37

### Dose Response in SKOV3 Cells as Determined by Western Blot

SKOV3 cells were plated in SKOV3 Growth Media (DMEM supplemented with 10% FBS and pen/strep) at a density of 4×10⁵ cells per well of 12 well plates. Twenty four hours later the media was removed and replaced with 1 ml media containing test compound and 0.1% DMSO and cells were returned to the incubator for 1 hr. At the end of the hour, the media was removed and the cells were washed with PBS, then lysed and scraped into 30 ul of Cell Extraction Buffer (Biosource, Camarillo, Calif.) plus Complete Protease Inhibitor and PhosStop Phosphatase Inhibitor (Roche, Indianapolis, Ind.).

Cell debris was spun down at 13,000×g for 1 minute and the supernatant was taken as the cell lysate. Protein concentration of the lysate was determined by BCA Assay (Pierce Biotechnology, Rockford, Ill.) and 50 ug of protein was loaded per well onto a NuPAGE Novex 4-12% Bis-Tris gel (Invitrogen, Carlsbad, Calif.) then transferred to Immobilon PVDF-FL (Millipore, Billerica, Mass.).

The blot was blocked in Odyssey Blocking Buffer (Li-Cor Biosciences, Lincoln, Nebr.) for 1 hr then incubated overnight at 4° C. with mouse anti-Akt (#2920) and rabbit anti-Phospho-Akt(Ser473) (#9271)(Cell Signaling Technology, Boston, Mass.) antibodies, both diluted 1:1000 in PBS/Odyssey Buffer (1:1)+0.1% Tween-20. The blots were washed 3 times 5 minutes in PBS+0.2% Tween-20 then incubated for 1 hr at room temperature with fluorescently labeled secondary antibodies (Li-Cor) diluted 1:10000 in PBS/Odyssey Buffer (1:1)+0.1% Tween-20.

The blots were washed 2 times for 5 minutes in PBS+0.2% Tween-20, once in distilled water, then scanned on an Odyssey machine (Li-Cor). Band intensity was determined using the Odyssey software and Phopho-Akt signal was normalized to total Akt within samples, then expressed as a percentage of the untreated Phospho-Akt signal.

Table 22 shows the dose response of selected compounds of this invention in the SKOV3 dose response assay as determined by Western blot. Compounds having an activity designated as “A” provided an EC₅₀≦10 nM; compounds having an activity designated as “B” provided an EC₅₀ of 10-100 nM; compounds having an activity designated as “C” provided an EC₅₀ of 100-1000 nM; and compounds having an activity designated as “D” provided an EC₅₀≧1000 nM.

TABLE 22
SKOV3 Dose Response as determined by Western blot

| Compound # | Immunoblot |
|---|---|
| II-a-3 | B |
| II-a-14 | B |
| II-a-22 | B |
| II-a-36 | B |
| II-a-64 | B |
| II-a-89 | B |
| II-a-112 | B |
| II-a-116 | B |
| II-a-142 | B |
| II-a-148 | A |
| II-a-154 | A |
| II-a-156 | A |
| II-a-172 | A |
| II-a-173 | A |
| II-a-176 | B |
| II-g-3 | C |
| II-g-6 | C |
| VII-13 | B |
| XII-2 | D |

### Example 38

Dose Response in SKOV3 Cells as Determined by in-Cell Western

SKOV3 cells were plated in SKOV3 Growth Media (DMEM supplemented with 10% FBS and pen/strep) at a density of 3×10⁴ cells per well of Costar #3603 black 96 well clear flat bottom plates. Twenty four hours later the media was removed and replaced with 100 ul media containing test compound or control compound and cells were returned to the incubator for 1 hr. At the end of the hour, the media was removed and the cells were washed once with PBS, then fixed for 20 minutes at room temperature in 4% formaldehyde in PBS. The formaldehyde was removed and cells were washed 5 times for 5 minutes with 100 ul of Permeabilization Buffer (PBS+0.1% Triton X-100) at room temperature with gentle shaking. The last wash was removed and replaced with 150 ul of Odyssey Blocking Buffer (Li-Cor, Lincoln, Nebr.) and incubated for 90 minutes at room temperature with gentle shaking.

The Blocking Buffer was then replaced with 50 ul of primary antibody mix (rabbit anti-Phospho-Akt(Ser473) at 1:100 (Cell Signaling Technology, Boston, Mass.) and mouse anti-tubulin at 1:5000 (Sigma Aldrich, St. Louis, Mo.) diluted in Odyssey Blocking Buffer) and incubated overnight at room temperature with gentle shaking.

The next morning, the antibody mix was removed and the wells were washed 5 times for 5 minutes with PBS+0.1% Tween-20. The last wash was replaced with 50 ul of secondary antibody mix (goat anti-rabbit-IRDye-680 and goat anti-mouse-IRDye-800 (Li-Cor), both diluted 1:1000 in Odyssey Blocking Buffer+0.2% Tween-20) and incubated for 1 hour at room temperature with gentle shaking. The antibody mix was removed and the wells were washed 5 times for 5 minutes in PBS+0.1% Tween-20, then 1 time with ddH₂O.

The plates were scanned on an Odyssey machine (Li-Cor) with a 3 mm focus offset at an intensity of 8 in both channels and the data was analyzed using the Odyssey software.

Table 23 shows the dose response of selected compounds of this invention in the SKOV3 in cell Western assay. Compounds having an activity designated as “A” provided an EC₅₀≦10 nM; compounds having an activity designated as “B” provided an EC₅₀ of 10-100 nM; compounds having an activity designated as “C” provided an EC₅₀ of 100-1000 nM; and compounds having an activity designated as “D” provided an EC₅₀≧1000 nM.

TABLE 23
SKOV3 In Cell Western Data

| | pAKT Inhibition |
|---|---|
| Compound # | in cell Western |
| GDC-941 | B |
| IX-ref | B |
| II-a-36 | C |
| II-a-37 | C |
| II-a-45 | A |
| II-a-14 | A |
| II^{R}-a-36 | C |
| II-a-112 | A |
| II-a-115 | C |
| II-a-116 | B |
| II-a-117 | B |
| II-a-118 | C |
| II-a-122 | C |
| II-a-123 | B |
| II-a-126 | A |
| II-a-127 | A |
| II-a-130 | B |
| II-a-132 | B |
| II-a-133 | B |
| II-a-137 | B |
| II-a-138 | C |
| II-a-139 | C |
| II-a-140 | C |
| II-a-141 | C |
| II-a-142 | B |
| II-a-143 | A |
| II-a-144 | C |
| II-a-148 | B |
| II-a-86 | A |
| II^{R}-a-148 | C |
| II-a-161 | A |
| II-a-3 | A |
| II-a-163 | B |
| II-a-164 | B |
| II-a-173 | B |
| II-a-174 | A |
| II-a-175 | A |
| V-20 | B |
| X-ref | A |
| X-1 | A |
| XI-ref | B |
| XI-3 | D |
| XII-4 | B |
| XII-5 | B |
| XII-39 | C |
| XII-41 | C |
| XII-42 | C |
| XII-46 | C |
| XII-47 | C |
| XII-48 | C |
| XII-49 | C |
| XII-50 | C |
| XII-51 | B |
| XII-52 | C |
| XII-54 | C |
| II-g-1 | B |
| II-g-2 | A |
| II-g-3 | A |
| VII-ref | B |
| VII-7 | C |
| VII-8 | C |
| VII-9 | C |
| VII-12 | B |
| VII-13 | B |

### Example 39

Washout Experiment with HCT116 Cells

HCT116 cells were plated overnight and then incubated for 1 hour with 5 μM (GDC-941), 1 μM (GSK-615, II-a-16, II-a-33, II-a-36, and II-a-37), or 0.5 μM (II-a-43, II-a-49, II-a-50, II-a-53, II-a-54, and II-a-55) of inhibitors. Cells were then washed every 2 hours with PBS. At each time point (t=0, 2, 4, 8 and 18 hours), cells were either lysed and the protein lysates recovered, or incubated in cell media for the next time point. Protein samples from every time point were then analyzed by Western blot. The results of this experiment with compounds listed above are depicted in FIG. 1.

### Example 40

Washout Experiment with PC3 Cells

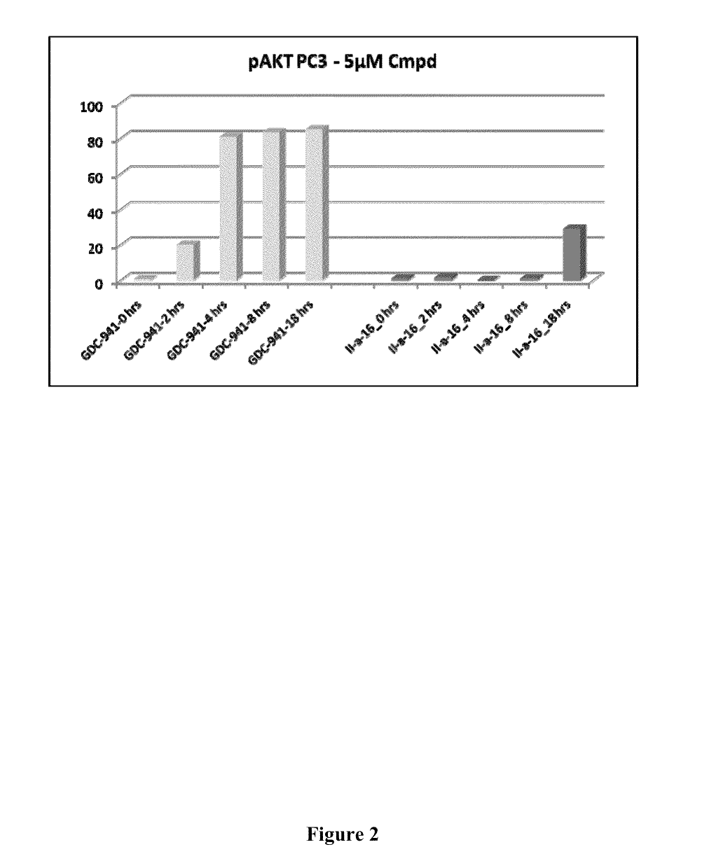

PC3 cells were plated overnight and then incubated for 1 hour with 5 μM of inhibitors. Cells were then washed every 2 hours with PBS. At each time point (t=0, 2, 4, 8 and 18 hours), cells were either lysed and the protein lysates recovered, or incubated in cell media for the next time point. Protein samples from every time point were then analyzed by Western blot. The results of this experiment with GDC-941 and II-a-16 are depicted in FIG. 2.

### Example 41

Washout Experiment with SKOV3 Cells as Determined by in-Cell Western

SKOV3 cells were plated in SKOV3 Growth Media (DMEM supplemented with 10% FBS and pen/strep) at a density of 2.5×10⁴ cells per well of Costar #3603 black 96 well clear flat bottom plates. Plates were set up in quadruplicate with one plate each for the 0, 1, 6 and 24 hour time points.

Twenty four hours later the media was removed and replaced with 100 ul media containing test compound or DMSO as a control and cells were returned to the incubator for 1 hr. At the end of the hour, the media was removed and the cells were washed 2 times with PBS. The PBS was removed from three of the plates, replaced with 100 ul of Growth Media and the plates were returned to the incubator. The fourth plate was taken as the 0 hour time point and developed as described for In-Cell Western Dose Response.

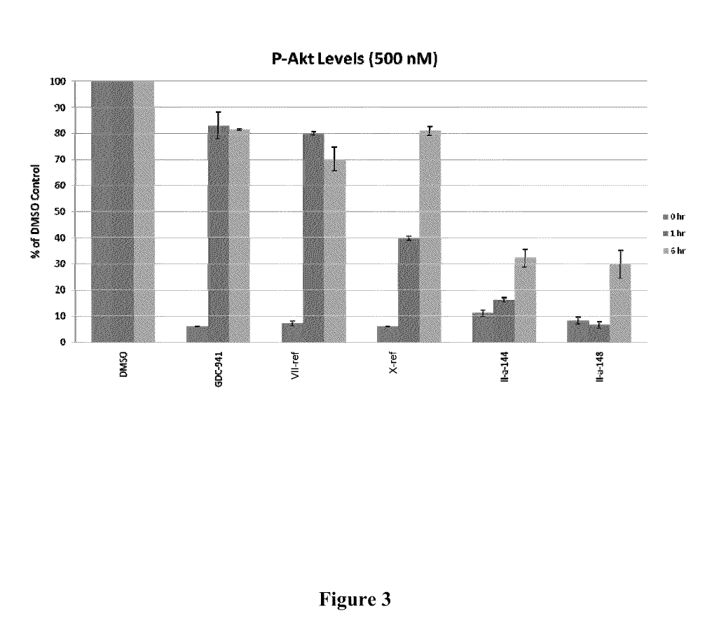

A half hour after the first wash, the media was removed from the remaining plates, replaced with 100 ul of fresh Growth Media and then the plates were returned to the incubator. At one hour after the first wash, one plate was taken as the 1 hour time point and developed as an In-Cell Western. The remaining two plates were washed two more times at one hour intervals and developed as In-Cell Westerns at 6 and 24 hours after the first wash. The results of this experiment with II-a-144 and II-a-148 are depicted in FIG. 3. The results show that II-a-144 and II-a-148 inhibit p-AKT for more than 6 h after removal from SKOV3 cells. Three reversible reference compounds show immediate return of activity.

### Example 42

### Mass Spectrometry for PI3K

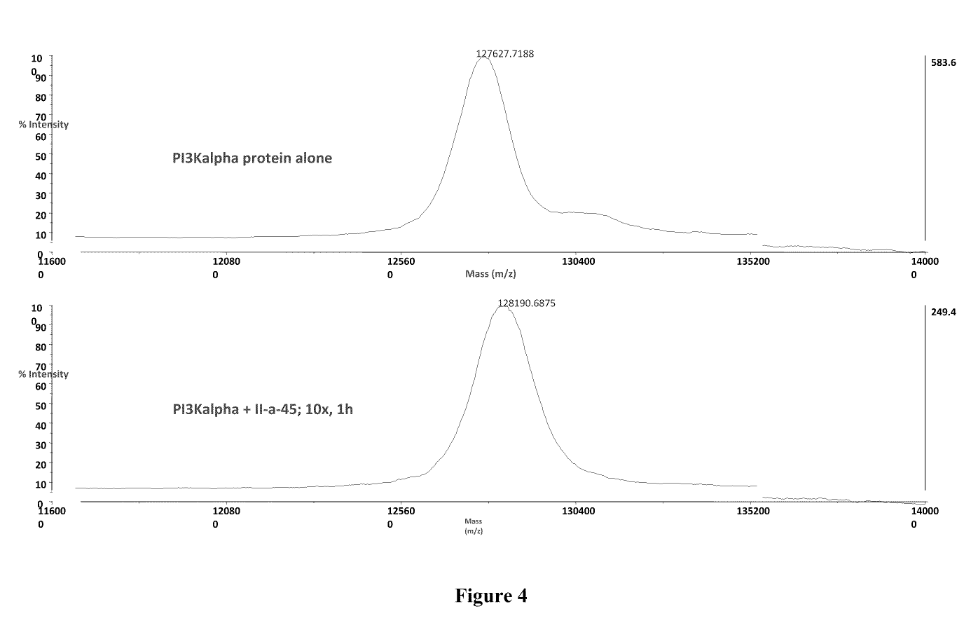

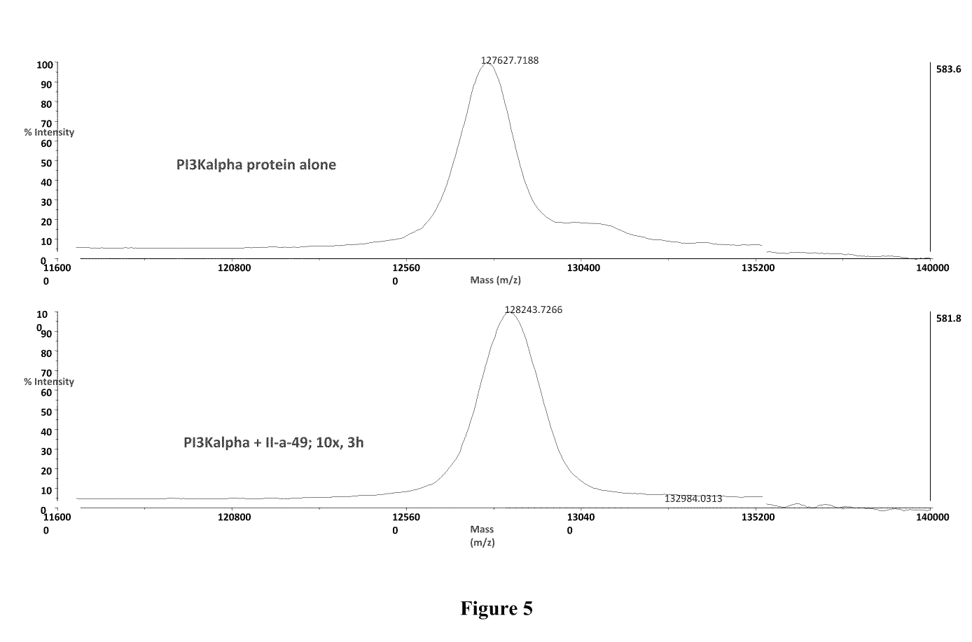

Intact PI3Kα (Johns Hopkins) was incubated for 3 hr at a 10× fold excess of II-a-45 or II-a-49 to protein. Aliquots (3 μL) of the samples were diluted with 10 μL of 0.1% TFA prior to micro C4 ZipTipping directly onto the MALDI target using Sinapinic acid as the desorption matrix (10 mg/ml in 0.1% TFA:Acetonitrile 50:50). Mass spectrometry traces are shown in FIG. 4 and FIG. 5. The top panels of FIGS. 4 and 5 shows the mass spec trace of the intact PI3Kα protein (m/z 127,627 Da). The bottom panels of FIGS. 3 and 4 shows mass spec trace when PI3Kα was incubated with II-a-45 (mw=518.64) or II-a-49 (mw=535.67). The centroid mass (m/z=128,190 Da) in the bottom panel of FIG. 4 shows a positive mass shift of 563 Da indicating complete modification of PI3Kα by II-a-45. The centroid mass (m/z=128,243 Da) in the bottom panel of FIG. 5 shows a positive mass shift of 616 Da indicating complete modification of PI3Kα by II-a-49. Other compounds that completely modify PI3Kα include II-a-16, II-a-33, II-a-36, II-a-37, II-a-43, II-a-50, II-a-53, II-a-54, and II-a-55.

### Example 43

### Mass Spectrometry for PI3K

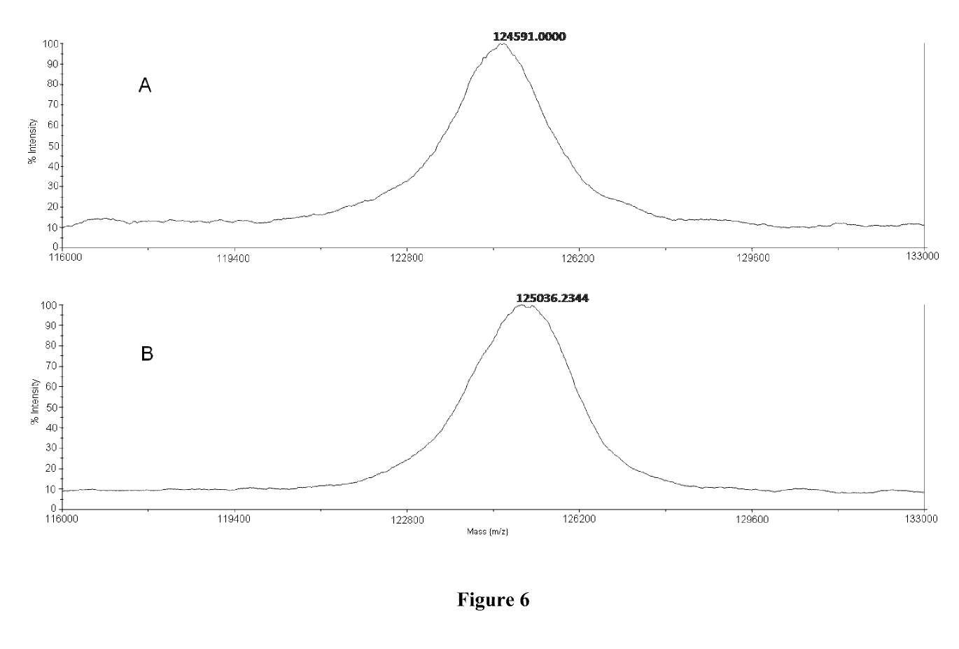

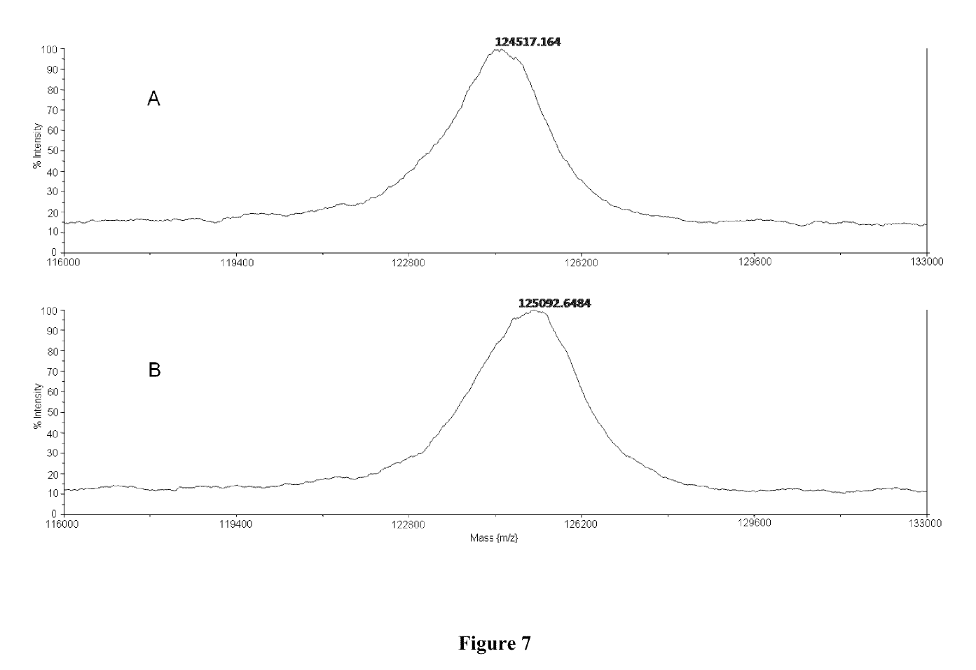

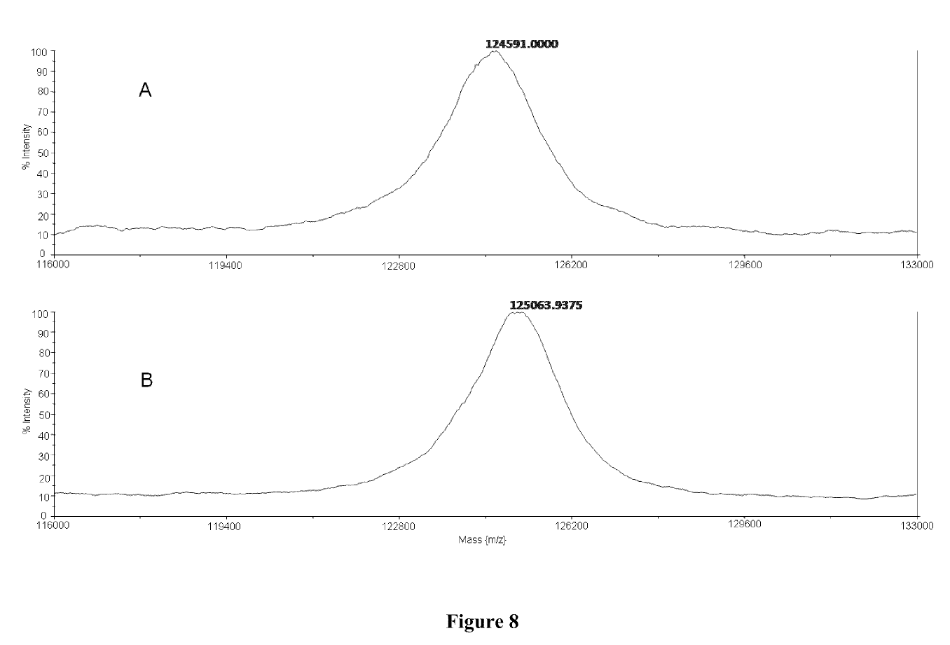

Intact PI3Kα (Millipore, 14-602) was incubated for 1 hr at a 10× fold excess of II-a-3, II-a-144, or II-a-148 to protein. Aliquots (5 μl) of the samples were diluted with 15 μl of 0.2% TFA prior to micro C4 ZipTipping directly onto the MALDI target using Sinapinic acid as the desorption matrix (10 mg/ml in 0.1% TFA:Acetonitrile 50:50). Mass spectrometry traces are shown in FIGS. 6, 7, and 8. Panel A of FIGS. 6, 7, and 8 shows the mass spec trace of the intact PI3Kα protein (m/z 124,951 Da). Panel B of FIGS. 6, 7, and 8 shows the mass spec trace when PI3Kα was incubated with II-a-3 (mw=573.72), II-a-144 (mw=591.69), or II-a-148 (mw=553.64) for 1 h. The centroid mass (m/z=125,036 Da) in Panel B of FIG. 6 shows a mass shift of 445 Da (78%), indicating complete modification of PI3Kα by II-a-3. The centroid mass (m/z=125,092 Da) in Panel B of FIG. 7 shows a mass shift of 575 Da (97%), indicating complete modification of PI3Kα by II-a-144. The centroid mass (m/z=125,063 Da) in Panel B of FIG. 8 shows a mass shift of 472 Da (85%), indicating complete modification of PI3Kα by II-a-148.

### Example 44

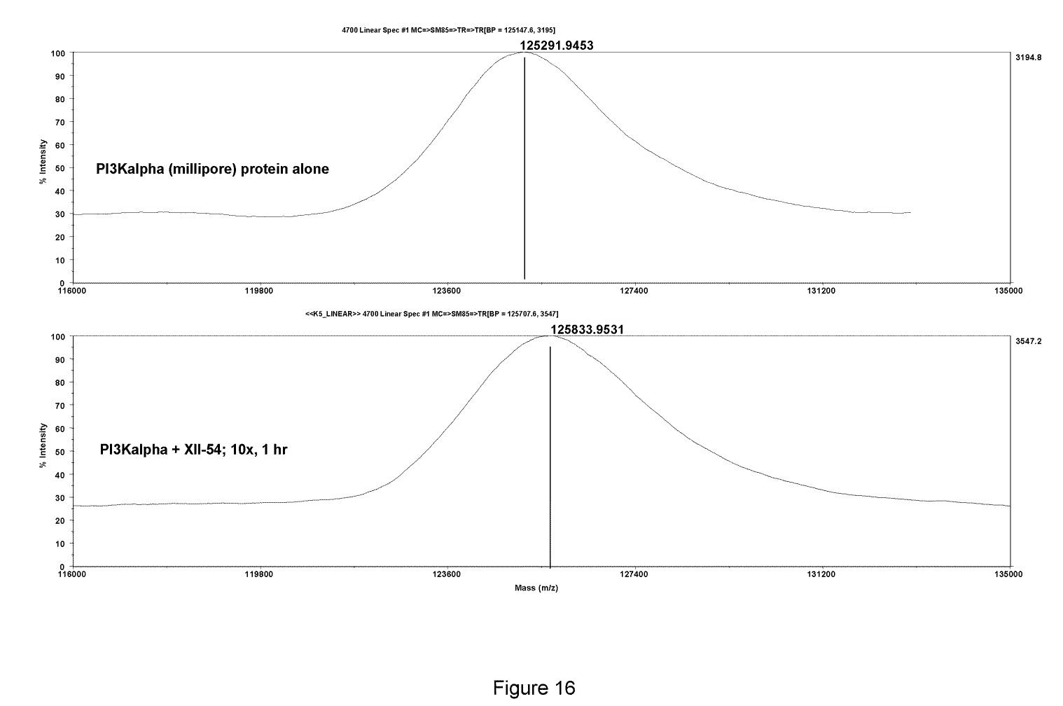

Using the protocol described in Example 43, certain compounds of formula XII were tested. A mass spectrometry trace for compound XII-54 is shown in FIG. 16. The top panel shows the mass spec trace of the intact PI3Kalpha protein (m/z=125,291 Da). The bottom panel shows the mass spec trace of PI3Kalpha incubated with XII-54 (mw=528.62) for 1 hr. The centroid mass (m/z=125,833 Da) shows a mass shift of 542 Da (103%), indicating modification of PI3Kalpha by XII-54. Other compounds that similarly modify PI3Kα include XII-15, XII-18, XII-42, XII-51, and XII-52.

### Example 45

### Trypsin Digest and MS-MS Analysis for II-a-3

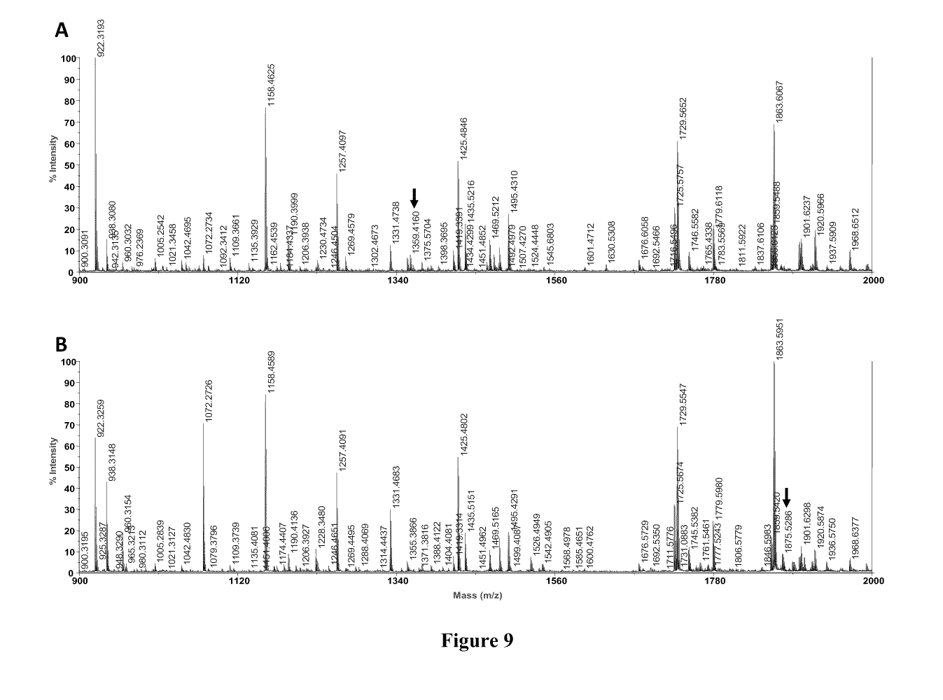

Intact PI3Kα (Millipore, 14-602) was incubated for 1 hr at a 10× fold excess of II-a-3 to protein. Following the reaction, 4 μg of control and II-a-3-treated PI3Kα was separated electrophoretically on a 4-12% BT gel and then stained with coomassie blue protein stain. The PI3Kα protein band was then excised and subjected to an in-gel trypsin digest by reducing the protein with DTT, alkylating the thiols with iodoacetamide, and then incubating the protein gel band with trypsin overnight in a 37° C. water bath. The digest was then stopped by addition of trifluoroacetic acid, and peptides were removed from gel band by sonicating with increasing amounts of acetonitrile (0%, 30%, & 60%). Peptides were then purified using C18 ziptips, spotted on the MALDI target plate with α-cyano-4-hydroxycinnamic acid as the desorption matrix (10 mg/ml in 0.1% TFA:Acetonitrile 50:50), and analyzed in reflectron mode. Panel A of FIG. 9 shows the trypsin digest profile for PI3Kα control and the arrow indicates the correct mass for peptide ⁸⁵³NSHTIMQIQCK⁸⁶³ (SEQ ID NO:14) with the Cys alkylated with an iodoacetamide. Panel B of FIG. 9 shows the trypsin digest profile for PI3Kα treated with II-a-3 prior to digestion and the arrow indicates the correct mass for peptide ⁸⁵³NSHTIMQIQCK⁸⁶³ (SEQ ID NO:14) with the Cys modified with a single II-a-3. Both peptides were selected for MSMS analysis to confirm the exact amino acid being modified.

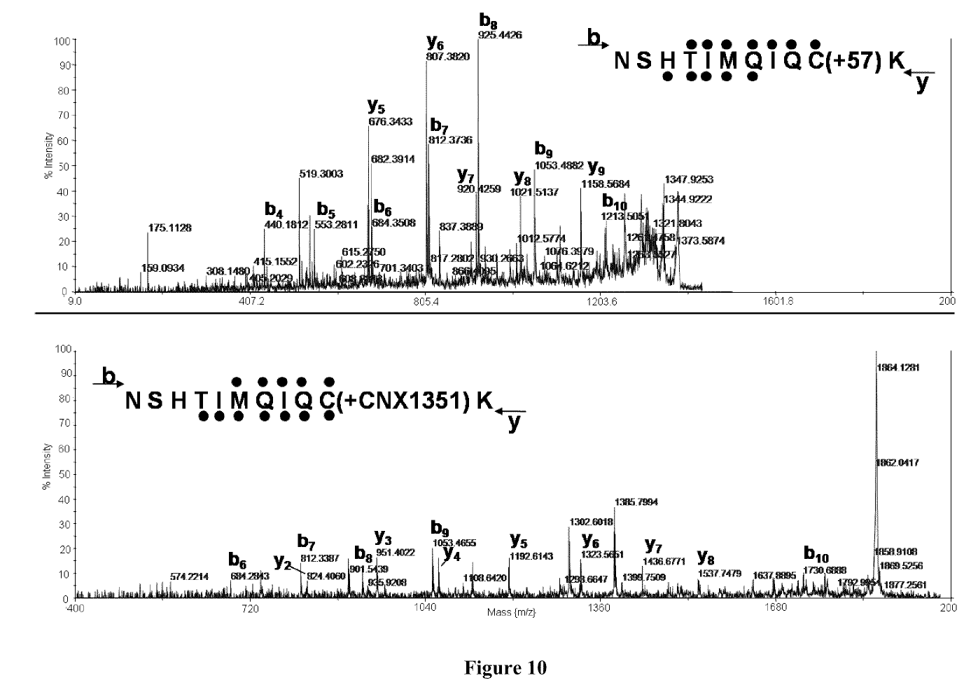

The peptide of interest was selected for MSMS analysis from both the control and II-a-3 treated PI3Kα. Panel A of FIG. 10 shows the MSMS spectrum of peptide ⁸⁵³NSHTIMQIQCK⁸⁶³ (SEQ ID NO:14) from the control digest where the Cys is alkylated by iodoacetamide during the digestion. Panel B of FIG. 10 shows the MSMS spectrum of peptide ⁸⁵³NSHTIMQIQCK⁸⁶³ (SEQ ID NO:14) from the II-a-3 treated PI3Kα digest where the Cys is modified by one II-a-3. The alignment of b and y ions confirms that Cys-862 is the amino acid that is modified by II-a-3.

### Example 46

### Trypsin Digest and MS-MS Analysis for II-a-144

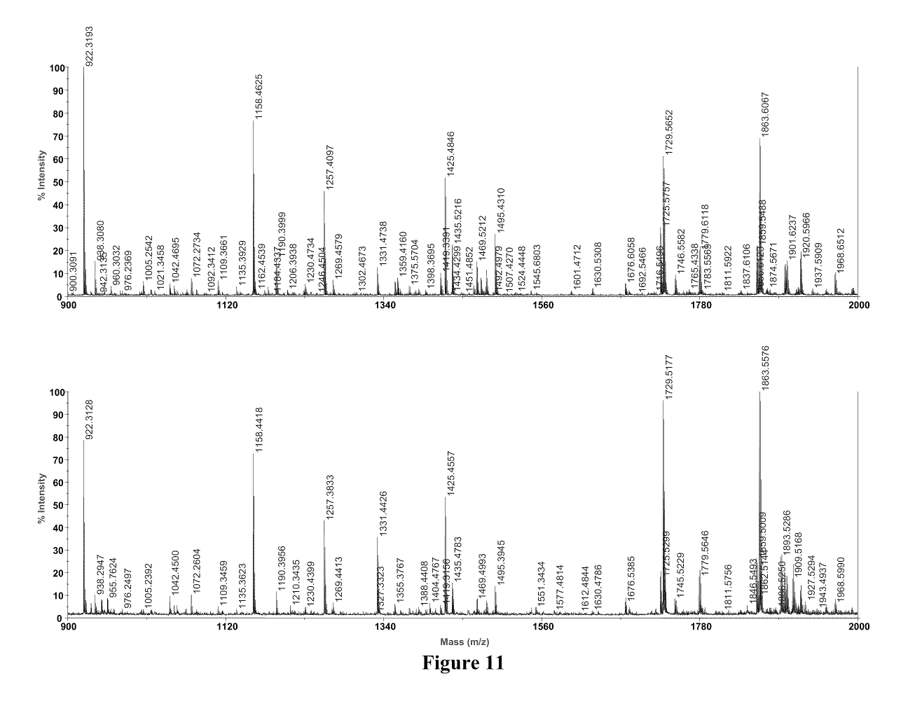

Intact PI3Kα (Millipore, 14-602) was incubated for 1 hr at a 10× fold excess of II-a-144 to protein. Following the reaction, 4 μg of control and II-a-144-treated PI3Kα was separated electrophoretically on a 4-12% BT gel and then stained with coomassie blue protein stain. The PI3Kα protein band was then excised and subjected to an in-gel trypsin digest by reducing the protein with DTT, alkylating the thiols with iodoacetamide, and then incubating the protein gel band with trypsin overnight in a 37° C. water bath. The digest was then stopped by addition of trifluoro acetic acid, and peptides were removed from gel band by sonicating with increasing amounts of acetonitrile (0%, 30%, & 60%). Peptides were then purified using C18 ziptips, spotted on the MALDI target plate with α-cyano-4-hydroxycinnamic acid as the desorption matrix (10 mg/ml in 0.1% TFA:Acetonitrile 50:50), and analyzed in reflectron mode. Panel A of FIG. 11 shows the trypsin digest profile for PI3Kα control and the arrow indicates the correct mass for peptide ⁸⁵³NSHTIMQIQCK⁸⁶³ (SEQ ID NO:14) with the Cys alkylated with an iodoacetamide. Panel B of FIG. 11 shows the trypsin digest profile for PI3Kα treated with II-a-144 prior to digestion and the arrow indicates the correct mass for peptide ⁸⁵³NSHTIMQIQCK⁸⁶³ (SEQ ID NO:14) with the Cys modified with a single II-a-144. Both peptides were selected for MSMS analysis to confirm the exact amino acid being modified.

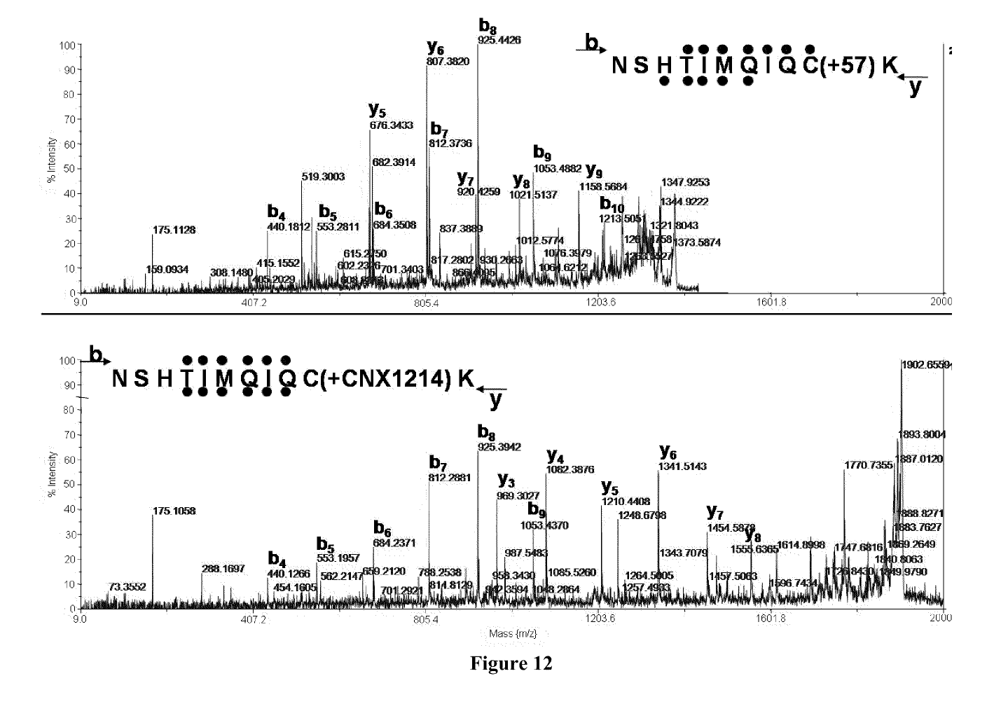

The peptide of interest was selected for MSMS analysis from both the control and II-a-144-treated PI3Kα. Panel A of FIG. 12 shows the MSMS spectrum of peptide ⁸⁵³NSHTIMQIQCK⁸⁶³ (SEQ ID NO:14) from the control digest where the Cys is alkylated by iodoacetamide during the digestion. Panel B of FIG. 12 shows the MSMS spectrum of peptide ⁸⁵³NSHTIMQIQCK⁸⁶³ (SEQ ID NO:14) from the II-a-144 treated PI3Kα digest where the Cys is modified by one II-a-144. The alignment of b and y ions confirms that Cys-862 is the amino acid that is modified by II-a-144.

### Example 47

### HCT-116 Cell Proliferation Assay

For the HCT116 Proliferation Assay, 3000 cells per well were plated in Growth Media (DMEM, 10% FBS, 1% 1-glutamine, 1% penicillin/streptomycin) in 96 well plates. The following day, compounds were added to duplicate wells at concentrations of 10 uM and 3-fold dilutions down to 40 nM. The plates were returned to the incubator for 72 hours and then the assays were developed using Cell Titer Glo (Promega, Madison, Wis.) according to manufacturer's instructions.

TABLE 24

| Compound # | EC₅₀ (μM) |
|---|---|
| GDC-941 | 1-10 |
| II-a-36 | 1-10 |
| II-a-43 | 0.1-1 |
| II-a-49 | 0.1-1 |
| II-a-50 | 0.1-1 |
| II-a-53 | 0.1-1 |
| II-a-55 | 0.1-1 |

### Example 48

### SK-OV-3 Cell Proliferation Assay

For the SK-OV-3 proliferation Assay, 5000 cells per well were plated in Growth Media (DMEM, 10% FBS, 1% 1-glutamine, 1% penicillin/streptomycin) in 96 well plates. The following day, compounds were added to duplicate wells at concentrations of 10 uM and 3-fold dilutions down to 40 nM. The plates were returned to the incubator for 72 hours and then the assays were developed using Cell Titer Glo (Promega, Madison, Wis.) according to manufacturer's instructions.

TABLE 25

| Compound # | EC₅₀ (μM) |
|---|---|
| GDC-941 | 1-10 |
| II-a-36 | 0.1-1 |
| II-a-43 | 0.1-1 |
| II-a-49 | 0.1-1 |
| II-a-50 | 0.1-1 |
| II-a-53 | 0.1-1 |
| II-a-55 | 1-10 |

### Example 49

### GI₅₀ Determinations in SKOV3 Cells

SKOV3 cells were plated in SKOV3 Proliferation Assay Media (DMEM supplemented with 5-10% FBS and pen/strep) at a density of 5000 cells in 180 ul volume per well in Costar #3610 white 96 well clear flat bottom plates, and incubated overnight in a humidified 37° C. incubator. A standard curve ranging from 10,000 to 50,000 cells was set up in a separate plate and allowed to adhere to the plate for 4-6 hours, at which time the plate was developed using Cell Titer-Glow (Promega, Madison, Wis.) according to manufacturer's instructions.

The next morning, 3-fold compound dilutions ranging from 10,000 nM to 40 nM were prepared in Proliferation Media containing 1% DMSO. 20 ul of each dilution was added to the SKOV3 cells plated the previous day resulting in a dose response curve from 1000 nM to 4 nM. The cells were incubated for 96 hours and then developed with Cell Titer Glo.

The cell numbers at the end of the assay were determined using the standard curve generated at the start of the assay. Growth inhibition was calculated using the following formulas and GI50s were determined by plotting the % growth inhibition vs. Log compound concentration in GraphPad.

%growth=100×(T−T₀)/(C−T₀)

T=Cell Number at end of assay
T₀=Cell Number at start of assay (5000)
C=Number of cells in DMSO controls at end of assay

%growth inhibition=100−%growth

Table 26 shows the dose response of selected compounds of this invention in the SKOV3 GI₅₀ assay. Compounds having an activity designated as “A” provided an GI₅₀≦10 nM; compounds having an activity designated as “B” provided an GI₅₀ of 10-100 nM; compounds having an activity designated as “C” provided an GI₅₀ of 100-1000 nM; and compounds having an activity designated as “D” provided an GI₅₀≧1000 nM.

TABLE 26
GI₅₀ Data

| Compound # | GI₅₀ |
|---|---|
| II-a-3 | B |
| II-a-86 | B |
| II-a-143 | C |
| II-a-144 | C |
| II-a-148 | B |
| II-a-158 | C |
| II-a-159 | C |
| II-a-160 | B |
| II-a-163 | C |
| II-g-2 | C |
| VII-8 | C |
| VII-9 | C |
| VII-10 | B |
| VII-11 | C |
| IX-5 | B |

### Example 50

### In Vivo Pharmacodynamic Evaluation of PI3Kα Covalent Inhibitor

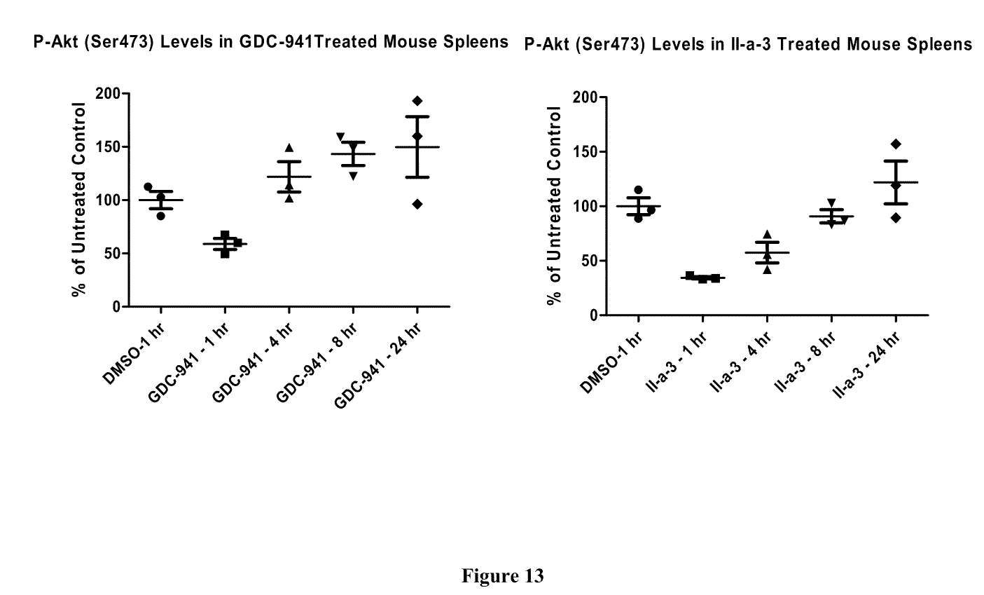

The in vivo experiment was performed at Vivisource (Waltham, Mass.). Nude mice (n=3/group) were given compound (reference compound GDC-0941 or II-a-3) delivered I.P. at 100 mg/Kg, once daily for 5 consecutive days. After delivery of the last dose, spleens from treated animals were harvested at 1 hour, 4 hour, 8 hour and 24 hour time points. Spleens were immediately frozen in liquid nitrogen. Samples were stored at −80° C. until processing for homogenates. Homogenates were made as described in Example 52. Homogenates were interrogated for P-Akt expression as described in Example 37. Results are shown in FIG. 13.

### Example 51

### Tumor Growth Inhibition In Vivo

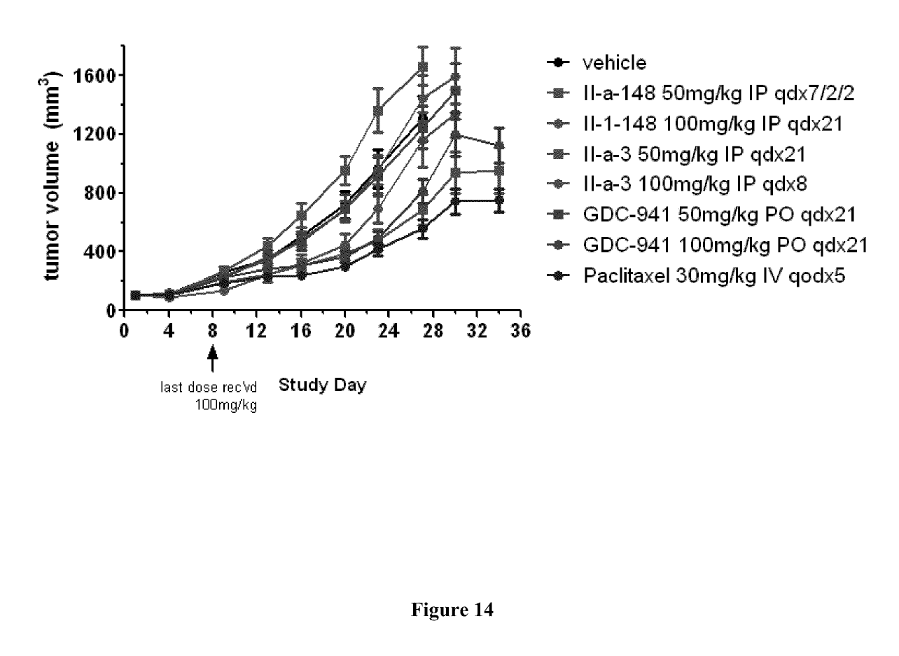

The in vivo experiment was performed at Piedmont Research Center (Research Triangle Park, N.C.). Nude mice were implanted with SKOV-3 tumors subcutaneously. Once the tumor size reached approximately 100 mm³, animals began receiving reference compound GDC-941, delivered orally, or II-a-3, delivered I.P., at 50-100 mg/Kg/QD. Dosing continued for 21 days. Tumor volume was measured twice a week. FIG. 14 shows results from a tumor growth inhibition assay with II-a-3 and II-a-148 compared with GDC-941 as well as paclitaxel. Inhibition of tumor growth in mice treated with II-a-3 or GDC-941 is shown in FIG. 14.

### Example 52

### In Vitro Occupancy

SKOV-3 cells were treated with GDC-941 or II-a-148 as described in Example 37. 150 ug of protein sample was added to a 0.2 ml tube and the volume brought up to 100 ul with IP Buffer from the Protein A/G Plate IP Kit (Pierce Biotechnology, Rockford, Ill.). XIV-a-3 was added at a concentration of 1 uM or XIV-a-4 was added at 50 nM and the tube was incubated at room temperature with rocking for 1 hr.

Protein A/G coated wells from the Protein A/G Plate IP Kit were washed 3× with 200 ul of IP Buffer. The wells were then coated with 4 ul rabbit anti-p110 alpha antibody #4249 (Cell Signaling Technology, Danvers, Mass.) plus 36 ul of IP Buffer per well. After incubating at room temperature with shaking for 1 hour, the wells were washed 5× with 200 ul of IP Buffer and the protein samples, preincubated with XIV-a-3, were added to the wells. The wells were incubated overnight at 4° C. with shaking.

The next morning, the wells were washed 5× with 200 ul of IP Buffer. The last wash was allowed to stand for 5 minutes before removal. The immuoprecipitate was eluted from the plate with 40 ul of Pierce Elution Buffer for 30 seconds, after which time the eluate was moved to a 1.5 ml tube containing 4 ul of Pierce Neutralization Buffer. 15 ul of NuPAGE LDS Sample Buffer and 6 ul of NuPAGE Sample Reducing Agent (Invitrogen, Carlsbad, Calif.) were added to each tube and the samples were incubated at 70° C. for 5 minutes.

20 ul of the IP eluate was loaded per well onto a NuPAGE Novex 4-12% Bis-Tris gel (Invitrogen), run at 150 volts for 35 minutes, then transferred to a nitrocellulose membrane. The blot was rinsed once in water, then incubated for 2 minutes in Qentix Solution 1 (Pierce Biotechnology) followed by 5 rinses in water. The blot was then incubated for 10 minutes in Qentix solution 2, rinsed 5 times in water then blocked in Odyssey Blocking Buffer (Li-Cor) for an hour.

The blot was then incubated overnight at 4° C. with rabbit anti-p110 alpha antibody (Epitomics, Burlingame, Calif.) diluted 1:2500 in PBS/Odyssey Buffer (1:1)+0.1% Tween-20. The blot was washed 3 times 5 minutes in PBS+0.2% Tween-20 then incubated for 1 hr at room temperature with streptavidin-AlexaFluor-680 (Invitrogen) diluted 1:1000 and fluorescently labeled goat anti-rabbit-IRDye800 (Li-Cor) diluted 1:10000 in PBS/Odyssey Buffer (1:1)+0.1% Tween-20.

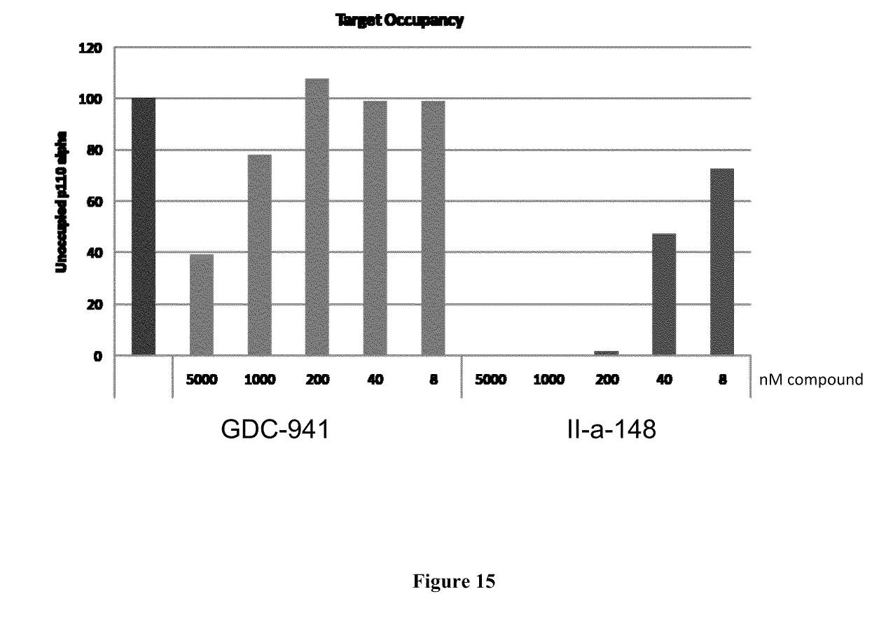

The blots were washed 2 times for 5 minutes in PBS+0.2% Tween-20, once in distilled water, then scanned on an Odyssey machine (Li-Cor, Lincoln, Nebr.). Band intensity was determined using the Odyssey software and streptavidin (Tool) signal was normalized to total p110 alpha signal within samples, then expressed as a percentage of the untreated signal. Results are shown in FIG. 15. Dose-dependent target occupancy is observed with irreversible compound II-a-148. The EC₅₀ for II-a-148 occupying p110alpha is ˜40 nM, which corresponds well with the P-AKT^{Ser473} EC₅₀. GDC-941 is a reversible compound that does not compete with the covalent probe.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A conjugate comprising one or more PI3 kinases having a cysteine residue, CysX, wherein the CysX is covalently, and irreversibly, bonded to an inhibitor, such that inhibition of the PI3 kinase is maintained, wherein CysX is selected from Cys862 of PI3K-alpha, Cys2243 of MTOR, Cys838 of PI3K-alpha, Cys869 of PI3K-gamma, Cys815 of PI3K-delta, Cys841 of PI3K-beta, Class 1A, Cys1119 of PI3K-beta, Class 2, Cys3683 of DNA-PK, Cys2770 of ATM-Kinase, Cys2753 of ATM-Kinase, Cys1840 of PI4KA, Cys1844 of PI4KA, or Cys1797 of PI4KA.

2. The conjugate according to claim 1, comprising one or more PI3 kinases having a cysteine residue selected from:
(a) Cys862 of PI3K-alpha; or
(b) any one or more of Cys869 of PI3K gamma, Cys838 of PI3K alpha, Cys815 of PI3K delta, Cys841 of PI3K beta, Class 1 or Cys1119 of PI3K beta, Class 2.

3. The conjugate of claim 1, wherein said conjugate is of formula C:
CysX-modifier-inhibitor moiety C
wherein:
the CysX is selected from Cys862 of PI3K-alpha, Cys2243 of MTOR, Cys838 of PI3K-alpha, Cys869 of PI3K-gamma, Cys815 of PI3K-delta, Cys841 of PI3K-beta, Class 1A, Cys1119 of PI3K-beta, Class 2, Cys3683 of DNA-PK, Cys2770 of ATM-Kinase, Cys2753 of ATM-Kinase, Cys1840 of PI4KA, Cys1844 of PI4KA, or Cys1797 of PI4KA;
the modifier is a bivalent group resulting from covalent bonding of a warhead group with the CysX of the PI3 kinase;
the warhead group is a functional group capable of covalently binding to CysX; and
the inhibitor moiety is a moiety that binds in the active site of the PI3 kinase.

4. The conjugate of claim 1, wherein said conjugate is of formula C-1:
Cys862-modifier-inhibitor moiety C-1
wherein:
the Cys862 is Cys862 of a PI3 kinase;
the modifier is a bivalent group resulting from covalent bonding of a warhead group with the Cys862 of the PI3 kinase;
the warhead group is a functional group capable of covalently binding to Cys862; and
the inhibitor moiety is a moiety that binds in the active site of the PI3 kinase.

5. The conjugate of claim 1, wherein said conjugate is of formula C-2:
CysX-modifier-inhibitor moiety C-2
wherein:
the CysX is any one or more of Cys869 of PI3K gamma, Cys838 of PI3K alpha, Cys815 of PI3K delta, Cys841 of PI3K beta, Class 1 or Cys1119 of PI3K beta, Class 2;
the modifier is a bivalent group resulting from covalent bonding of a warhead group with the CysX of the PI3 kinase;
the warhead group is a functional group capable of covalently binding to CysX; and
the inhibitor moiety is a moiety that binds in the active site of the PI3 kinase.

6. The conjugate of claim 2, wherein the inhibitor moiety is of formula I-i: 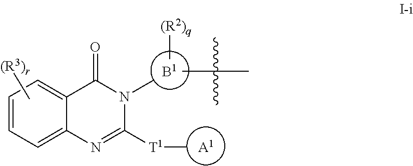
wherein the wavy bond indicates the point of attachment to the cysteine via the modifier;
Ring A¹ is an optionally substituted group selected from an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
Ring B¹ is selected from phenyl, a 3-8 membered saturated or partially unsaturated carbocyclic ring, a 3-8 membered saturated or partially unsaturated heterocyclic ring having 1-2 heteroatoms independently selected from nitrogen, oxygen, or sulfur, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T¹ is a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
q and r are each independently 0-4; and
each R² and R³ is independently R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

7. The conjugate of claim 2, wherein the inhibitor moiety is of formula II-i: 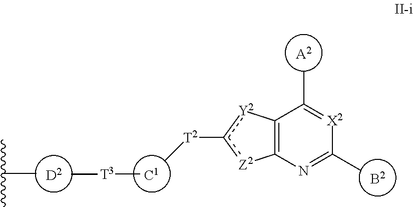
wherein the wavy bond indicates the point of attachment to the cysteine via the modifier;
X² is CH or N;
Y² and Z² are independently CR⁴, C, NR⁵, N, O, or S, as valency permits; [symbol]
represents a single or double bond, as valency permits;
R¹ is a warhead group;
Ring A² is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R⁴ is —R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
R⁵ is —R, —SO₂R, —SOR, —C(O)R, —CO₂R, or —C(O)N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B² is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T² is a covalent bond or a bivalent straight or branched, saturated or unsaturated C₁₋₆ hydrocarbon chain wherein one or more methylene units of T² are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C¹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T³ is a covalent bond or a bivalent straight or branched, saturated or unsaturated C₁₋₆ hydrocarbon chain wherein one or more methylene units of T³ are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring D² is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

8. The conjugate of claim 2, wherein the inhibitor moiety is of either of formula II-i-a or II-i-b: 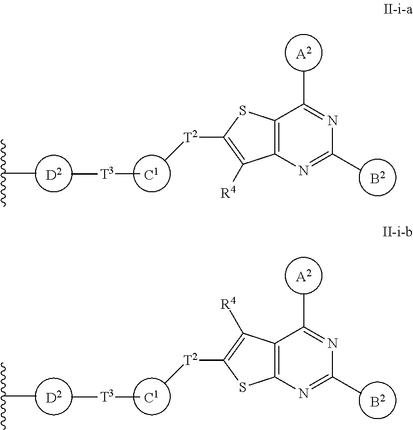
wherein the wavy bond indicates the point of attachment to the cysteine via the modifier;
Ring A² is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R⁴ is —R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B² is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T² is a covalent bond or a bivalent straight or branched, saturated or unsaturated C₁₋₆ hydrocarbon chain wherein one or more methylene units of T² are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C¹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T³ is a covalent bond or a bivalent straight or branched, saturated or unsaturated C₁₋₆ hydrocarbon chain wherein one or more methylene units of T³ are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring D² is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

9. The conjugate of claim 2, wherein the inhibitor moiety is of either of formula II-i-c or II-i-d: 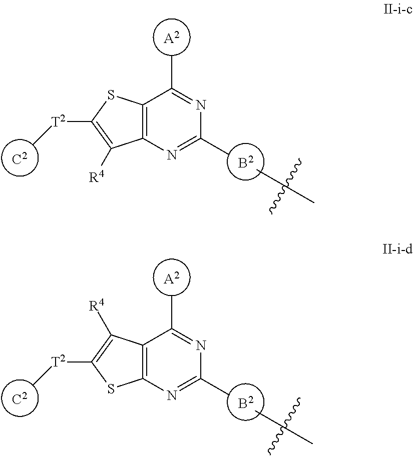
wherein the wavy bond indicates the point of attachment to the cysteine via the modifier;
Ring A² is an optionally substituted ring selected from a 4-8 membered saturated or partially unsaturated heterocyclic ring having one or two heteroatoms independently selected from nitrogen, oxygen, or sulfur, or a 5-10 membered saturated or partially unsaturated bridged bicyclic heterocyclic ring having at least one nitrogen, at least one oxygen, and optionally 1-2 additional heteroatoms independently selected from nitrogen, oxygen, or sulfur;
R⁴ is R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B² is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T² is a covalent bond or a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring C² is hydrogen or an optionally substituted ring selected from a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

10. The conjugate of claim 2, wherein the inhibitor moiety is of either of formula II-i-e or II-i-f: 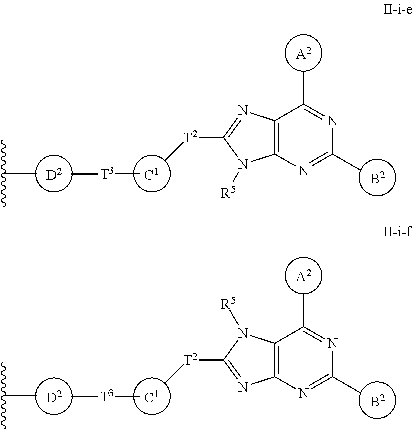
wherein the wavy bond indicates the point of attachment to the cysteine via the modifier;
Ring A² is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R⁵ is R, —SO₂R, —SOR, —C(O)R, —CO₂R, or —C(O)N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B² is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T² is a covalent bond or a bivalent straight or branched, saturated or unsaturated C₁₋₆ hydrocarbon chain wherein one or more methylene units of T² are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C¹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T³ is a covalent bond or a bivalent straight or branched, saturated or unsaturated C₁₋₆ hydrocarbon chain wherein one or more methylene units of T³ are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring D² is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

11. The conjugate of claim 2, wherein the inhibitor moiety is of either of formula II-i-g or II-i-h: 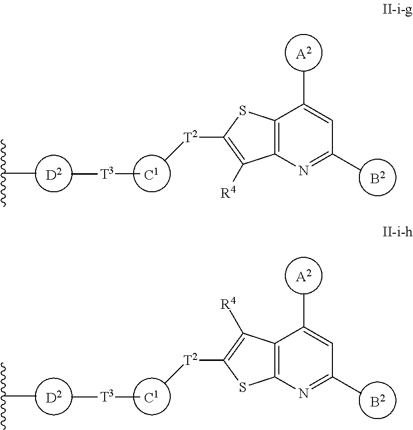
wherein the wavy bond indicates the point of attachment to the cysteine via the modifier;
Ring A² is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R⁴ is —R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B² is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T² is a covalent bond or a bivalent straight or branched, saturated or unsaturated C₁₋₆ hydrocarbon chain wherein one or more methylene units of T² are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C¹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T³ is a covalent bond or a bivalent straight or branched, saturated or unsaturated C₁₋₆ hydrocarbon chain wherein one or more methylene units of T³ are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring D² is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

12. The conjugate of claim 2, wherein the inhibitor moiety is of formula III-i: 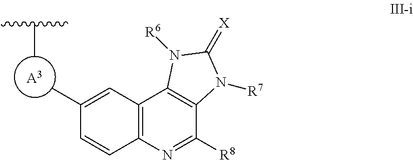
wherein the wavy bond indicates the point of attachment to the cysteine via the modifier;
X is O or S;
R⁶ is an optionally substituted group selected from phenyl, napthyl, a 6-membered heteroaryl ring having 1-2 nitrogens, or an 8-10 membered bicyclic heteroaryl ring having 1-3 heteroatoms independently selected from nitrogen, oxygen, or sulfur;
R⁷ is an optionally substituted C₁₋₆ aliphatic group;
R⁸ is hydrogen or —NHR′;
R′ is independently hydrogen or an optionally substituted C₁₋₆ aliphatic group; and
Ring A³ is an optionally substituted group selected from phenyl, naphthyl, a 6-membered heteroaryl ring having 1-2 nitrogens, or an 8-10 membered bicyclic heteroaryl ring having 1-3 nitrogens.

13. The conjugate of claim 2, wherein the inhibitor moiety is of formula IV-i: 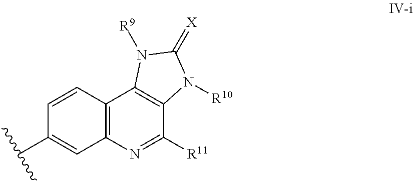
wherein the wavy bond indicates the point of attachment to the cysteine via the modifier;
X is O or S;
R⁹ is an optionally substituted group selected from phenyl, napthyl, a 6-membered heteroaryl ring having 1-2 nitrogens, or an 8-10 membered bicyclic heteroaryl ring having 1-3 heteroatoms independently selected from nitrogen, oxygen, or sulfur;
R¹⁰ is an optionally substituted C₁₋₆ aliphatic group;
R¹¹ is hydrogen or —NHR′; and
R′ is independently hydrogen or an optionally substituted C₁₋₆ aliphatic group.

14. The conjugate of claim 2, wherein the inhibitor moiety is of formula V-i-a or V-i-b: 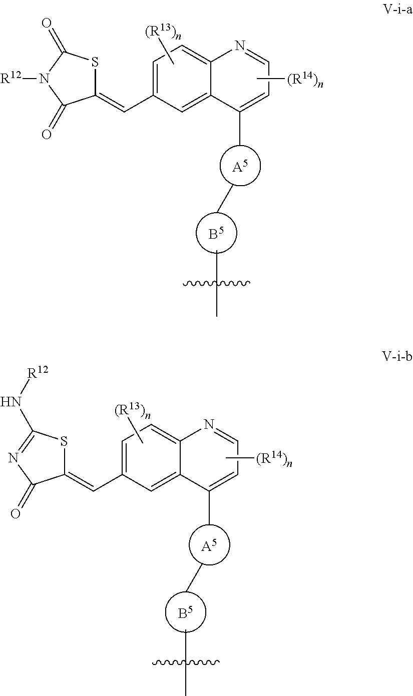
wherein the wavy bond indicates the point of attachment to the cysteine via the modifier;
R¹² is an hydrogen or an optionally substituted group selected from C₁₋₆ aliphatic, —(CH₂)ₘ-(3-7 membered saturated or partially unsaturated carbocyclic ring), —(CH₂)ₘ-(7-10 membered saturated or partially unsaturated bicyclic carbocyclic ring), —(CH₂)ₘ-(4-7 membered saturated or partially unsaturated heterocyclic ring having 1-2 heteroatoms independently selected from nitrogen, oxygen, or sulfur), —(CH₂)ₘ-(7-10 membered saturated or partially unsaturated bicyclic heterocyclic ring having 1-3 heteroatoms independently selected from nitrogen, oxygen, or sulfur), —(CH₂)ₘ-phenyl, —(CH₂)ₘ-(8-10 membered bicyclic aryl ring), —(CH₂)ₘ-(5-6 membered heteroaryl ring having 1-3 heteroatoms independently selected from nitrogen, oxygen, or sulfur), or —(CH₂)ₘ-(8-10 membered bicyclic heteroaryl ring having 1-4 heteroatoms independently selected from nitrogen, oxygen, or sulfur);
each R¹³ and R¹⁴ is independently —R″, halogen, —NO₂, —CN, —OR″, —SR″, —N(R″)₂, —C(O)R″, —CO₂R″, —C(O)C(O)R″, —C(O)CH₂C(O)R″, —S(O)R″, —S(O)₂R″, —C(O)N(R″)₂, —SO₂N(R″)₂, —OC(O)R″, —N(R″)C(O)R″, —N(R″)N(R″)₂, —N(R″)C(═NR″)N(R″)₂, —C(═NR″)N(R″)₂, —C═NOR″, —N(R″)C(O)N(R″)₂, —N(R″)SO₂N(R″)₂, —N(R″)SO₂R″, or —OC(O)N(R″)₂;
each R″ is independently hydrogen or an optionally substituted group selected from C₁₋₆ aliphatic, a 3-7 membered saturated or partially unsaturated carbocyclic ring, a 7-10 membered saturated or partially unsaturated bicyclic carbocyclic ring, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; or
two R″ groups on the same nitrogen are taken together with the nitrogen to which they are attached to form an optionally substituted 5-8 membered saturated, partially unsaturated, or aromatic ring having 1-4 heteroatoms independently selected from nitrogen, oxygen, or sulfur;
m is an integer from 0 to 6, inclusive;
each n is independently 0, 1, or 2;
Ring A⁵ is an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; and
Ring B⁵ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

15. The conjugate of claim 2, wherein the inhibitor moiety is of formula VI-i-a or VI-i-b: 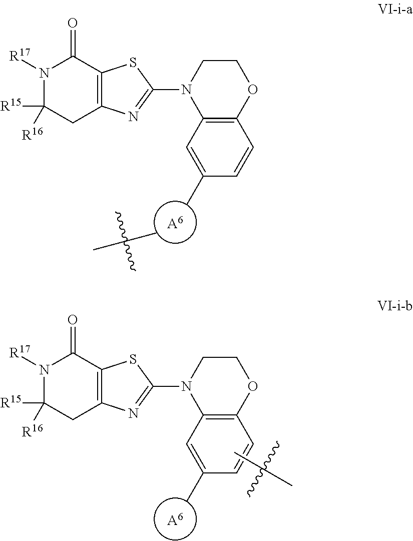
wherein the wavy bond indicates the point of attachment to the cysteine via the modifier;
R¹⁵ is hydrogen or C₁₋₆ alkyl;
R¹⁶ is hydrogen or an optionally substituted group selected from C₁₋₆ alkyl, C₁₋₆ alkoxy, or (C₁₋₆ alkylene)-R¹⁸; or
R¹⁵ and R¹⁶ are taken together with the intervening carbon to form an optionally substituted ring selected from a 3-7 membered carbocyclic ring or a 4-7 membered heterocyclic ring having 1-2 heteroatoms independently selected from nitrogen, oxygen, or sulfur;
R¹⁷ is hydrogen or C₁₋₆ alkyl;
R¹⁸ is a 3-7 membered saturated or partially unsaturated carbocyclic ring, a 7-10 membered saturated or partially unsaturated bicyclic carbocyclic ring, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, a 8-10 membered bicyclic aryl ring, a 5-6 membered heteroaryl ring having 1-3 heteroatoms independently selected from nitrogen, oxygen, or sulfur, or a 8-10 membered bicyclic heteroaryl ring having 1-4 heteroatoms independently selected from nitrogen, oxygen, or sulfur; and
Ring A⁶ is absent or an optionally substituted group selected from a 4-7 membered heterocyclic ring having 1-2 heteroatoms independently selected from nitrogen, oxygen, or sulfur, or a 5-6 membered heteroaryl ring having 1-3 heteroatoms independently selected from nitrogen, oxygen, or sulfur.

16. The conjugate of claim 2, wherein the inhibitor moiety is of formula VII-i: 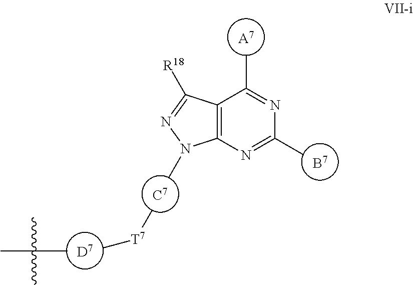
wherein the wavy bond indicates the point of attachment to the cysteine via the modifier;
Ring A⁷ is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R¹⁸ is R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B⁷ is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T⁷ is a covalent bond or a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C⁷ is an optionally substituted ring selected from a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; and
Ring D⁷ is absent or an optionally substituted ring selected from a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

17. The conjugate of claim 2, wherein the inhibitor moiety is of formula VIII-i: 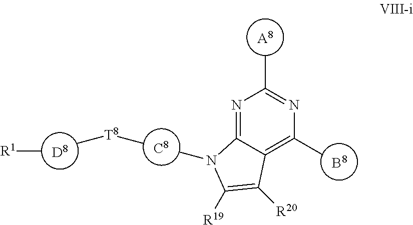
wherein the wavy bond indicates the point of attachment to the cysteine via the modifier;
Ring A⁸ is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R¹⁹ and R²⁰ are independently R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B⁸ is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T⁸ is a covalent bond or a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C⁸ is an optionally substituted ring selected from a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; and
Ring D⁸ is absent or an optionally substituted ring selected from a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

18. The conjugate of claim 2, wherein the inhibitor moiety is of formula IX-i: 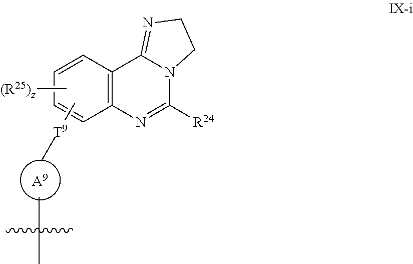
wherein the wavy bond indicates the point of attachment to the cysteine via the modifier;
T⁹ is a covalent bond or a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring A⁹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
R²⁴ and R²⁵ are independently R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur; and
z is 0, 1, or 2.

19. The conjugate of claim 2, wherein the inhibitor moiety is of formula X-i: 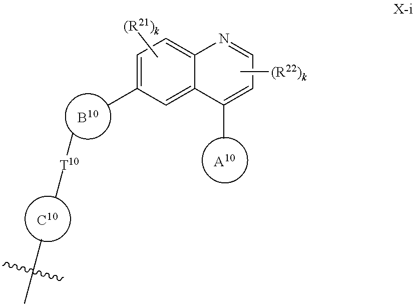
wherein the wavy bond indicates the point of attachment to the cysteine via the modifier;
each R²¹ and R²² is independently —R″, halogen, —NO₂, —CN, —OR″, —SR″, —N(R″)₂, —C(O)R″, —CO₂R″, —C(O)C(O)R″, —C(O)CH₂C(O)R″, —S(O)R″, —S(O)₂R″, —C(O)N(R″)₂, —SO₂N(R″)₂, —OC(O)R″, —N(R″)C(O)R″, —N(R″)N(R″)₂, —N(R″)C(═NR″)N(R″)₂, —C(═NR″)N(R″)₂, —C═NOR″, —N(R″)C(O)N(R″)₂, —N(R″)SO₂N(R″)₂, —N(R″)SO₂R″, or —OC(O)N(R″)₂;
each R″ is independently hydrogen or an optionally substituted group selected from C₁₋₆ aliphatic, a 3-7 membered saturated or partially unsaturated carbocyclic ring, a 7-10 membered saturated or partially unsaturated bicyclic carbocyclic ring, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; or
two R″ groups on the same nitrogen are taken together with the nitrogen to which they are attached to form an optionally substituted 5-8 membered saturated, partially unsaturated, or aromatic ring having 1-4 heteroatoms independently selected from nitrogen, oxygen, or sulfur;
each k is independently 0, 1, or 2;
Ring A¹⁰ is an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
Ring B¹⁰ is an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T¹⁰ is a covalent bond or a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring C¹⁰ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

20. The conjugate of claim 2, wherein the inhibitor moiety is of formula XI-i: 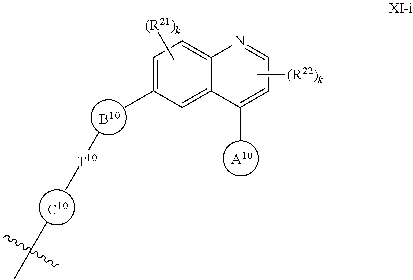
wherein the wavy bond indicates the point of attachment to the cysteine via the modifier;
X¹¹ is CH or N;
Ring A¹¹ is an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
each R²³ is independently —R^{a}, halogen, —NO₂, —CN, —OR^{b}, —SR^{b}, —N(R^{b})₂, —C(O)R^{a}, —CO₂R^{a}, —C(O)C(O)R^{a}, —C(O)CH₂C(O)R^{a}, —S(O)R^{a}, —S(O)₂R^{a}, —C(O)N(R^{a})₂, —SO₂N(R^{a})₂, —OC(O)R^{a}, —N(R^{a})C(O)R^{a}, —N(R^{a})N(R^{a})₂, —N(R^{a})C(═NR^{a})N(R^{a})₂, —C(═NR^{a})N(R^{a})₂, —C═NOR^{a}, —N(R^{a})C(O)N(R^{a})₂, —N(R^{a})SO₂N(R^{a})₂, —N(R^{a})SO₂R^{a}, or —OC(O)N(R^{a})₂;
each R^{a} is independently hydrogen, C₁₋₆ aliphatic, phenyl, a 3-7 membered saturated or partially unsaturated carbocyclic ring, a 7-10 membered saturated or partially unsaturated bicyclic carbocyclic ring, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; or
two R^{a} groups on the same nitrogen are taken together with the nitrogen to which they are attached to form an optionally substituted 5-8 membered saturated, partially unsaturated, or aromatic ring having 1-4 heteroatoms independently selected from nitrogen, oxygen, or sulfur;
each R^{b} is independently hydrogen, C, aliphatic, a 3-7 membered saturated or partially unsaturated carbocyclic ring, a 7-10 membered saturated or partially unsaturated bicyclic carbocyclic ring, a 4-7 membered saturated or partially unsaturated heterocyclic ring having 1-2 heteroatoms independently selected from nitrogen, oxygen, or sulfur, or a 7-10 membered saturated or partially unsaturated bicyclic heterocyclic ring having 1-3 heteroatoms independently selected from nitrogen, oxygen, or sulfur; or
two R^{b} groups on the same nitrogen are taken together with the nitrogen to which they are attached to form an optionally substituted 5-8 membered saturated, partially unsaturated, or aromatic ring having 1-4 heteroatoms independently selected from nitrogen, oxygen, or sulfur;
w is 0, 1, or 2;
Ring B¹¹ is an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T¹¹ is a covalent bond or a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring C¹¹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

21. The conjugate of claim 2, wherein the inhibitor moiety is of formula XII-i: 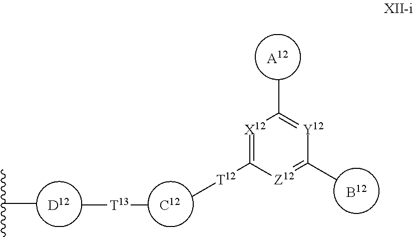
wherein the wavy bond indicates the point of attachment to the cysteine via the modifier;
R¹ is a warhead group;
X¹² is CR²⁶ or N;
Y¹² is CR²⁷ or N;
Z¹² is CR²⁸ or N;
wherein at least one of X¹², Y¹², and Z¹² is N;
Ring A¹² is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R²⁶, R²⁷, and R²⁸ are independently R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B¹² is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T¹² is a covalent bond or a bivalent straight or branched, saturated or unsaturated C₁₋₆ hydrocarbon chain wherein one or more methylene units of T¹² are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C¹² is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged or spiro bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T¹³ is a covalent bond or a bivalent straight or branched, saturated or unsaturated C₁₋₆ hydrocarbon chain wherein one or more methylene units of T¹³ are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring D¹² is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

22-45. (canceled)

46. A compound of formula I: 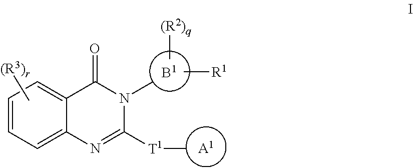
or a pharmaceutically acceptable salt thereof, wherein:
Ring A¹ is an optionally substituted group selected from an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
Ring B¹ is selected from phenyl, a 3-8 membered saturated or partially unsaturated carbocyclic ring, a 4-8 membered saturated or partially unsaturated heterocyclic ring having 1-2 heteroatoms independently selected from nitrogen, oxygen, or sulfur, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
R¹ is a warhead group;
T¹ is a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
q and r are each independently 0-4; and
each R² and R³ is independently R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

47-54. (canceled)

55. A compound of formula II-a or II-b: 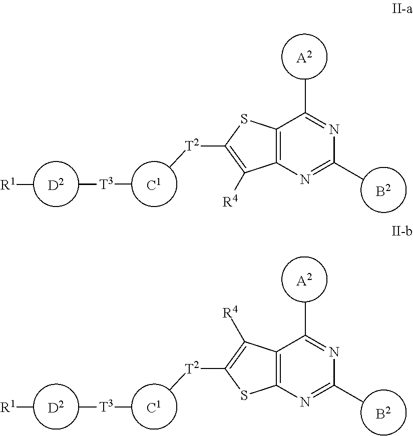
or a pharmaceutically acceptable salt thereof, wherein:
R¹ is a warhead group;
Ring A² is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R⁴ is —R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B² is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T² is a covalent bond or a bivalent straight or branched, saturated or unsaturated C₁₋₆ hydrocarbon chain wherein one or more methylene units of T² are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C¹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T³ is a covalent bond or a bivalent straight or branched, saturated or unsaturated C₁₋₆ hydrocarbon chain wherein one or more methylene units of T³ are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring D² is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

56. The compound according to claim 55, wherein Ring B² is an optionally substituted 8-10 membered bicyclic heteroaryl ring having 1-4 heteroatoms independently selected from nitrogen, oxygen, or sulfur.

57. The compound according to claim 56, wherein Ring B² is an optionally substituted 8-10 membered bicyclic heteroaryl ring having 2 nitrogen atoms.

58. The compound according to claim 55, wherein Ring B² is 1H-indazolyl, optionally substituted phenyl, phenol, or optionally substituted pyridyl or pyrimidyl.

59-62. (canceled)

63. The compound according to claim 55, wherein Ring A² is an optionally substituted 6-membered saturated or partially unsaturated heterocyclic ring having one or two heteroatoms independently selected from nitrogen, oxygen, or sulfur.

64. The compound according to claim 63, wherein Ring A² is optionally substituted morpholinyl.

65. (canceled)

66. The compound according to claim 64, wherein Ring A² is selected from the following:

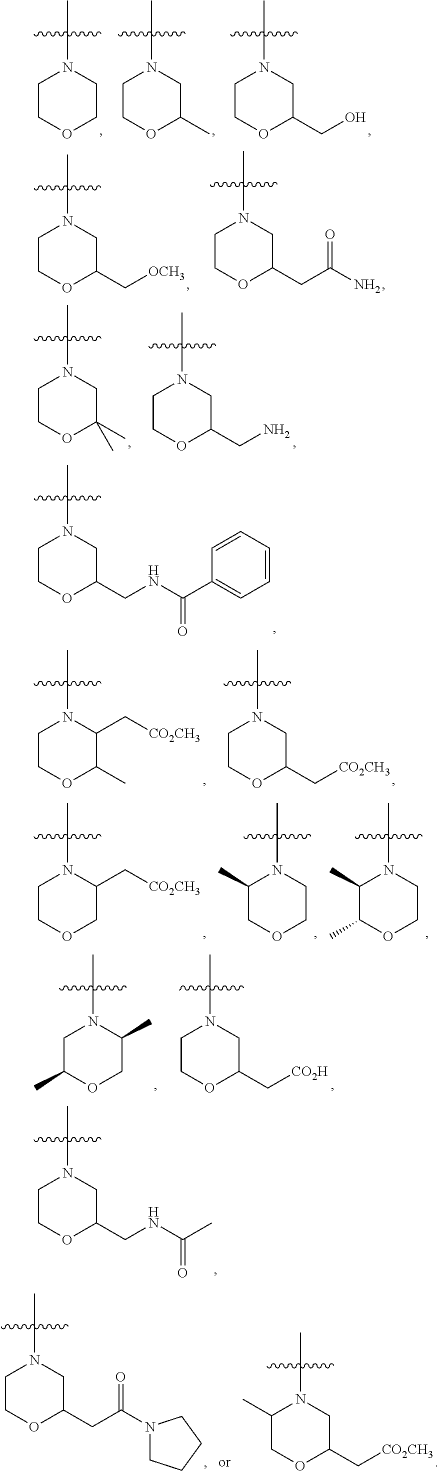

67. The compound according to claim 55, wherein Ring A² is a bridged, bicyclic morpholino group.

68. The compound according to claim 65, wherein Ring A² is selected from:

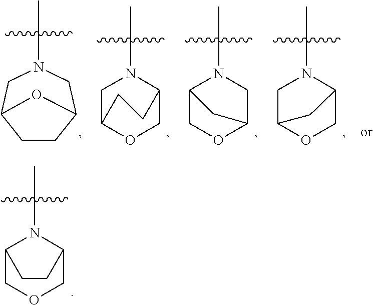

69. The compound according to claim 55, wherein Ring A² is selected from:

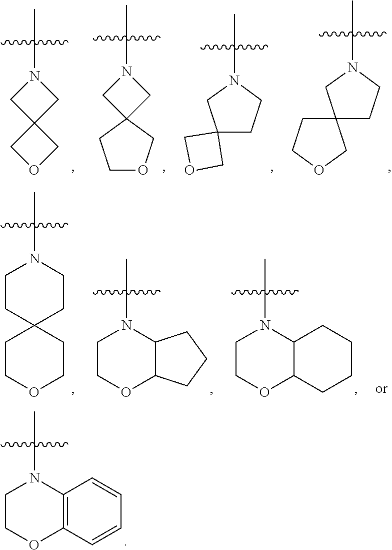

70. The compound according to claim 55, wherein T² is a bivalent, straight, saturated or unsaturated C₁₋₆ hydrocarbon chain.

71. (canceled)

72. The compound according to claim 70, wherein T² is —CH₂—, —C≡C—, or —CH₂C≡C—.

73. The compound according to claim 55, wherein T² is a covalent bond or —C(O)—.

74-77. (canceled)

78. The compound according to claim 55, wherein T² is a covalent bond, methylene, or a C₂₋₄ hydrocarbon chain wherein one methylene unit of T² is replaced by —C(O)NH—.

79. (canceled)

80. The compound according to claim 55, wherein Ring C¹ is an optionally substituted 6-membered saturated heterocyclic ring having one or two heteroatoms independently selected from nitrogen, oxygen, or sulfur.

81. The compound according to claim 80, wherein Ring C¹ is a piperazinyl or piperidinyl ring.

82. (canceled)

83. The compound according to claim 55, wherein Ring C¹ is a tetrahydropyridyl ring, a phenyl ring, or a cyclohexyl ring.

84-85. (canceled)

86. The compound according to claim 55, wherein T³ is a bivalent, straight, saturated C₁₋₆ hydrocarbon chain.

87. (canceled)

88. The compound according to claim 86, wherein T³ is —CH₂— or —CH₂CH₂—.

89. The compound according to claim 55, wherein T³ is —C(O)— or a covalent bond.

90. (canceled)

91. The compound according to claim 55, wherein Ring D² is optionally substituted 6-membered saturated or partially unsaturated heterocyclic ring having one or two heteroatoms independently selected from nitrogen, oxygen, or sulfur.

92. The compound according to claim 91, wherein Ring D² is tetrahydropyridyl, piperidinyl or piperazinyl.

93. (canceled)

94. The compound according to claim 55, wherein Ring D² is phenyl.

95. The compound according to claim 55, wherein Ring D² is absent.

96. The compound according to claim 55, wherein 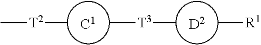
is selected from

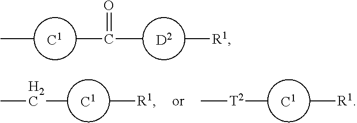

97. The compound according to claim 96, wherein 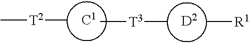
comprises a spacer group having about 9 to about 11 atoms.

98. The compound according to claim 55, wherein the compound has one or more, more than one, or all of the features selected from:
a) Ring A² is optionally substituted morpholinyl;
b) Ring B² is an optionally substituted group selected from indazolyl, aminopyrimidinyl, or phenol;
c) 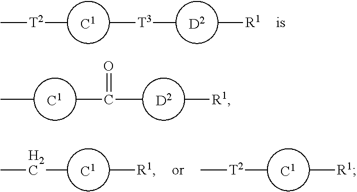
and
d) 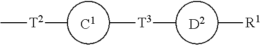
comprises a spacer group having about 9 to about 11 atoms.

99. The compound according to claim 55, wherein the compound has one or more, more than one, or all of the features selected from:
a) Ring A² is optionally substituted morpholinyl;
b) Ring B² is an optionally substituted 8-10 membered bicyclic heteroaryl ring having 1-2 nitrogen atoms, optionally substituted phenyl, or an optionally substituted 5-6 membered heteroaryl ring having 1-2 nitrogen atoms;
c) T² is a covalent bond, methylene, or a C₂₋₄ hydrocarbon chain wherein one methylene unit of T² is replaced by —C(O)NH—;
d) Ring C¹ is phenyl, or an optionally substituted 6-membered saturated, partially unsaturated, or aromatic heterocyclic ring having 1-2 nitrogens;
e) T³ is a covalent bond or —C(O)—; and
f) Ring D² is absent or phenyl.

100. The compound according to claim 55, wherein the compound has one or more, more than one, or all of the features selected from:
a) Ring A² is optionally substituted morpholinyl;
b) Ring B² is an optionally substituted group selected from indazolyl, phenol, or aminopyrimidine;
c) T² is a covalent bond, methylene, or a C₃ hydrocarbon chain wherein one methylene unit of T² is replaced by —C(O)NH—;
d) Ring C¹ is phenyl, piperazinyl, piperidinyl, or tetrahydropyridyl;
e) T³ is a covalent bond or —C(O)—; and
f) Ring D² is absent or phenyl.

101. The compound according to claim 55, wherein the compound is selected from the group consisting of:

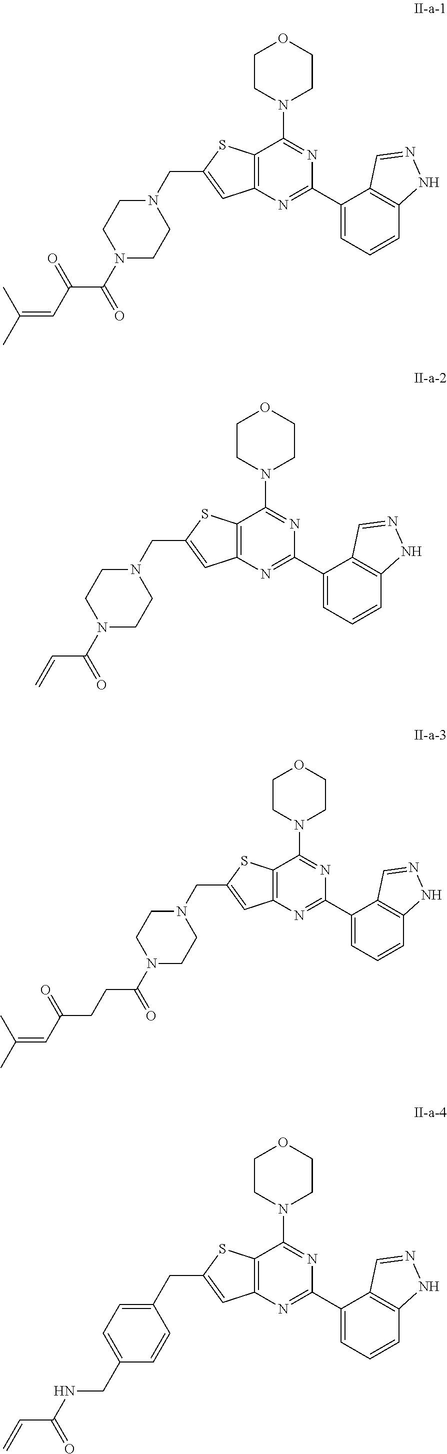

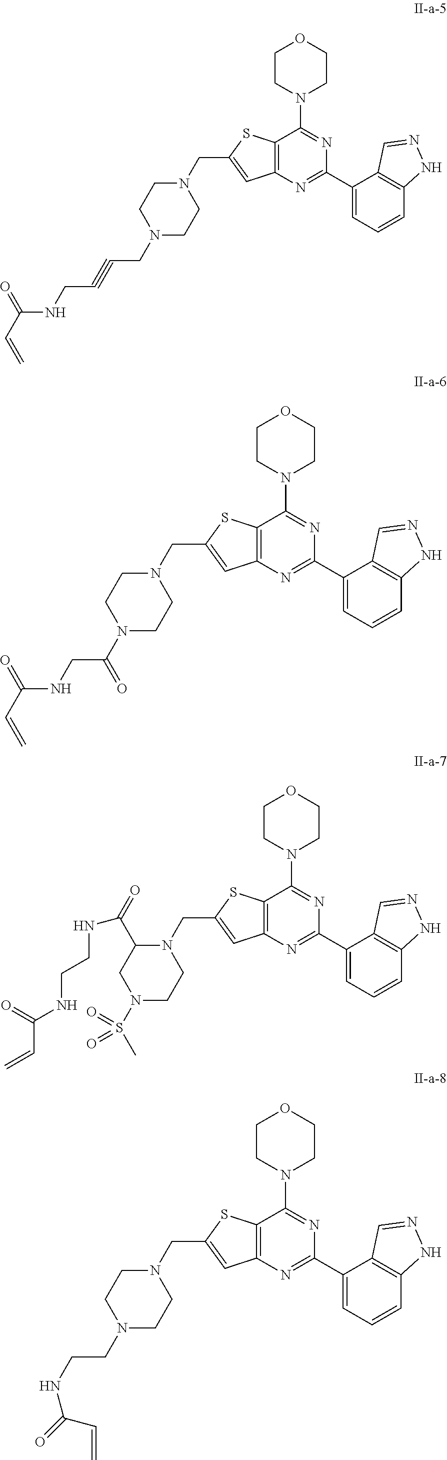

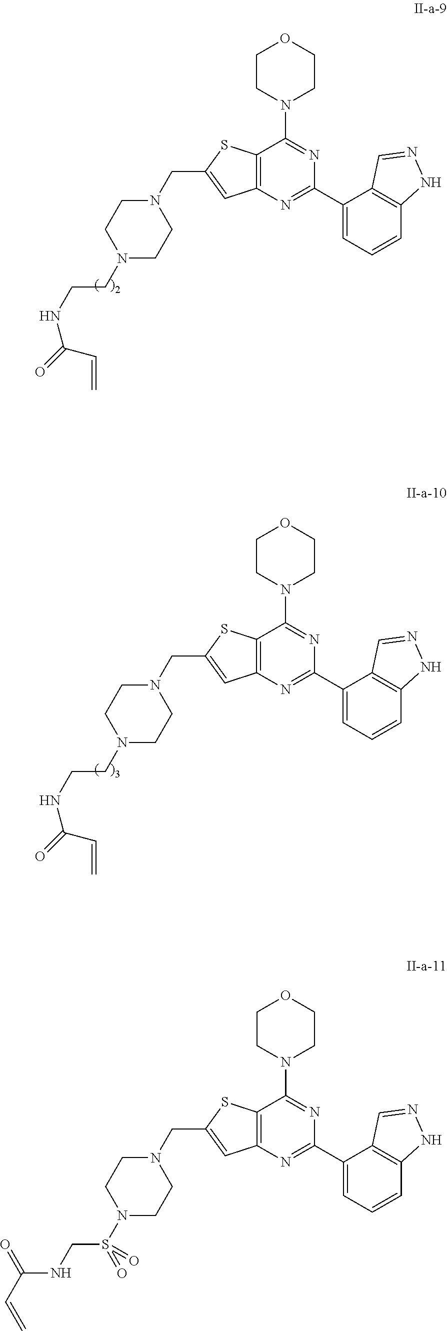

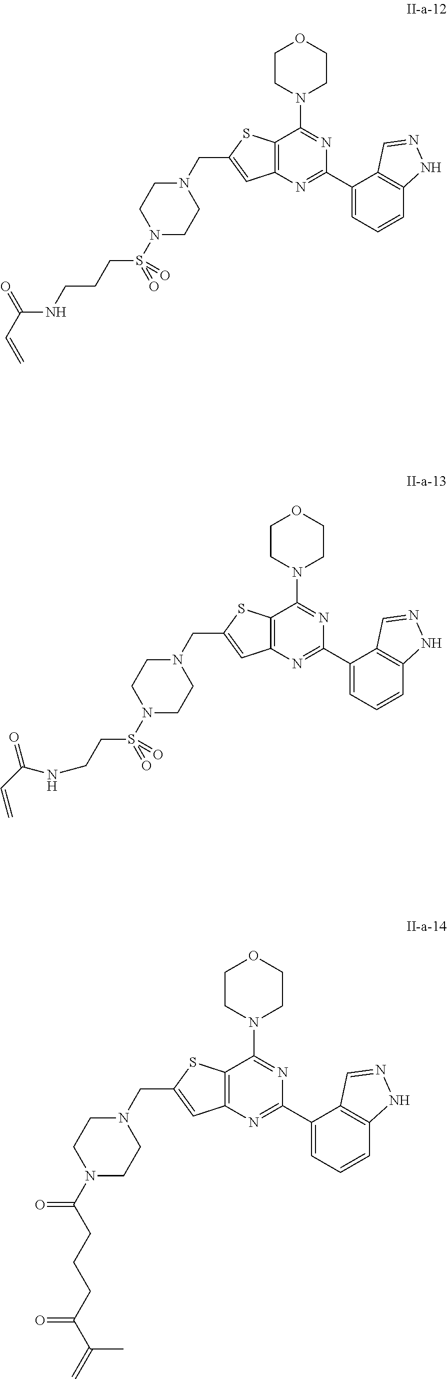

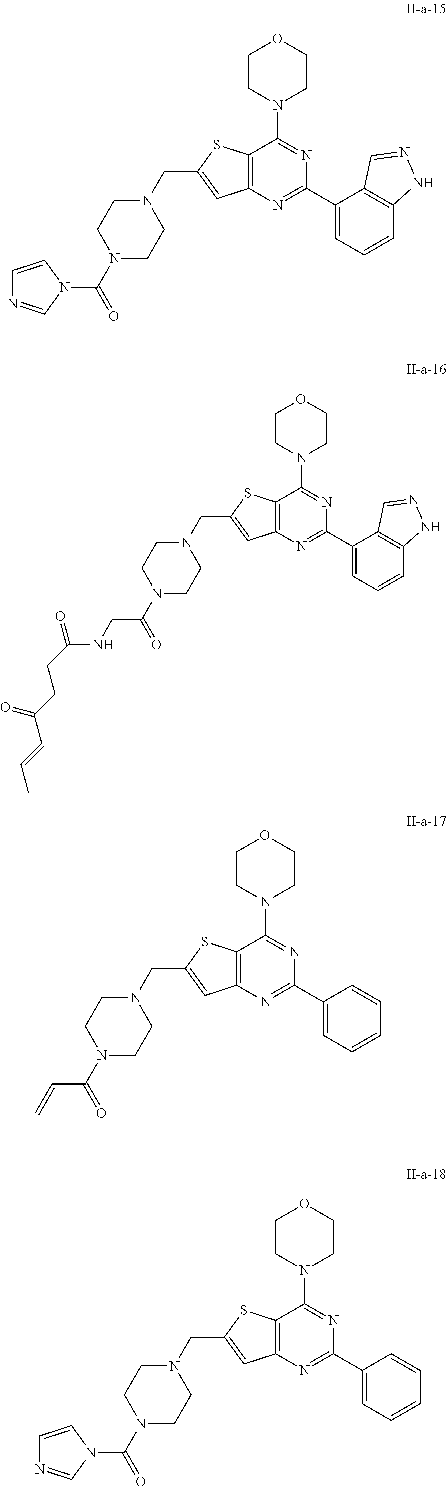

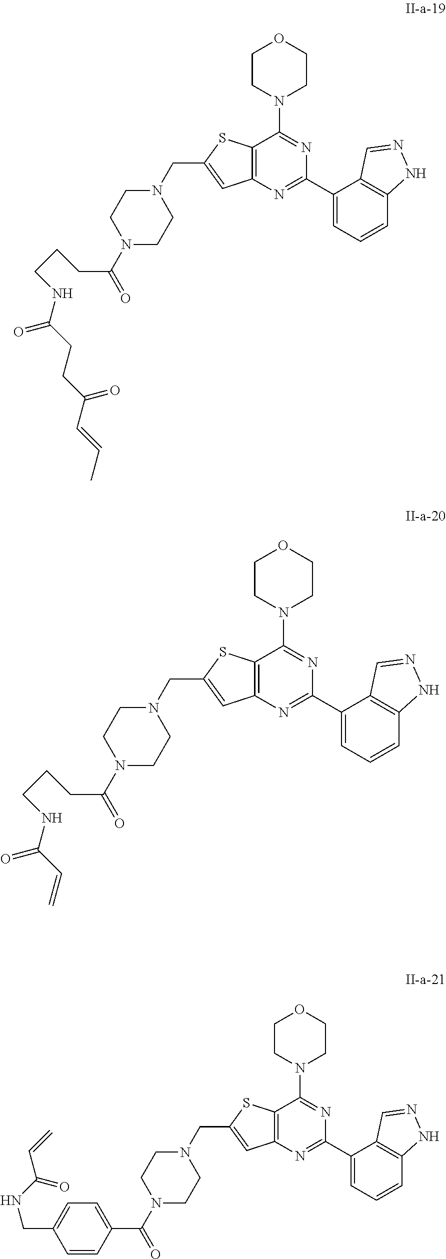

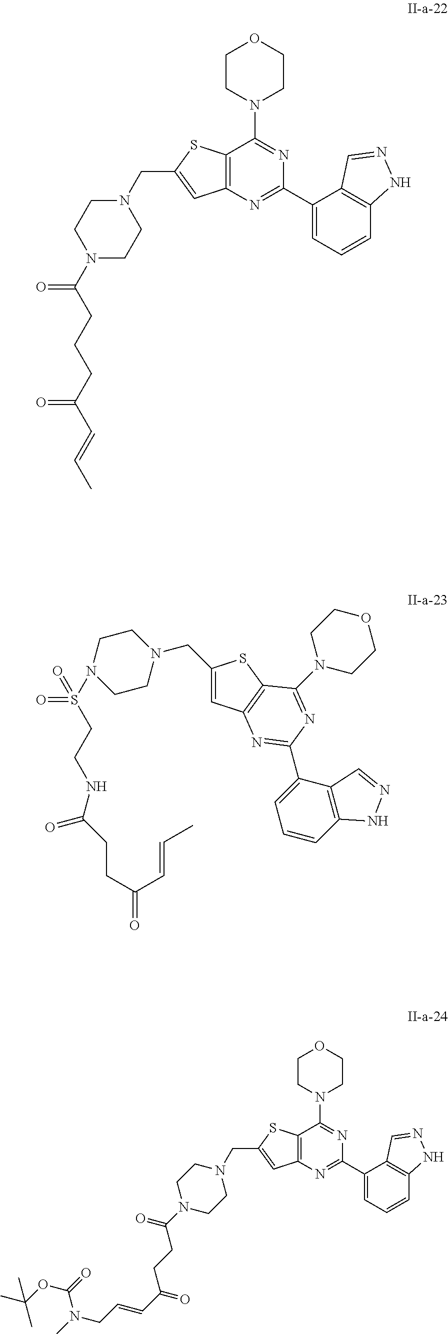

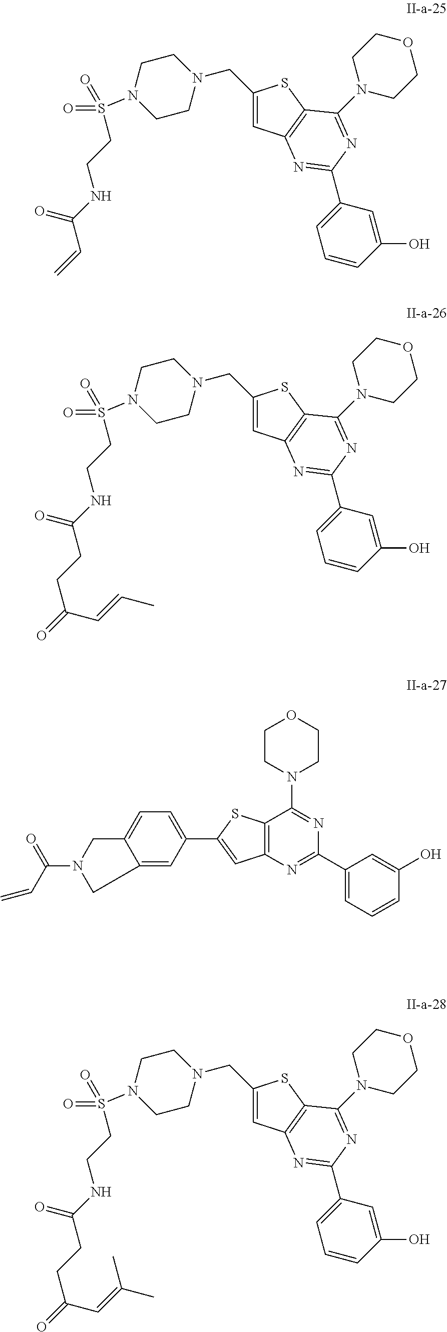

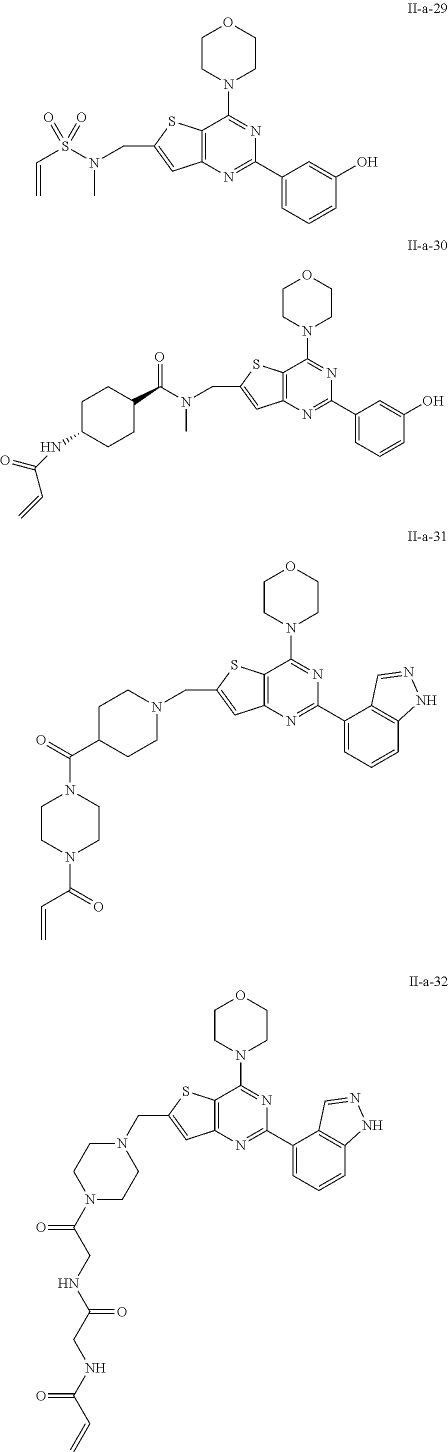

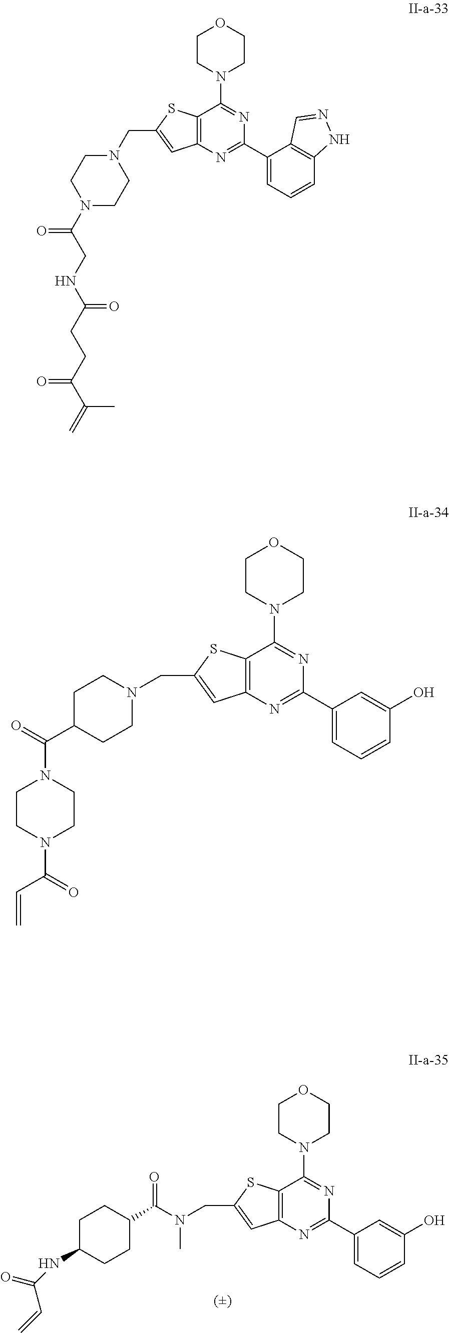

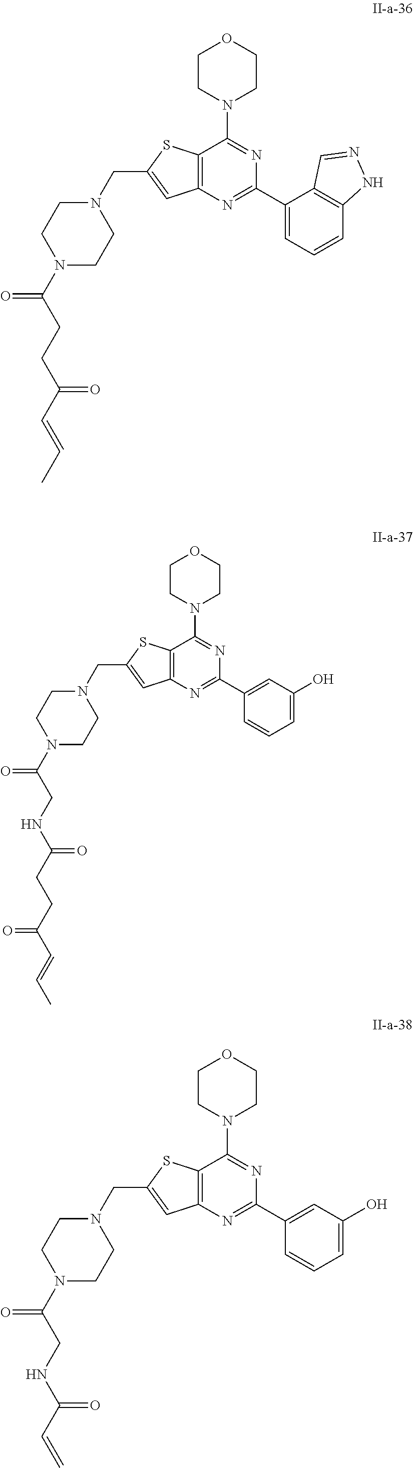

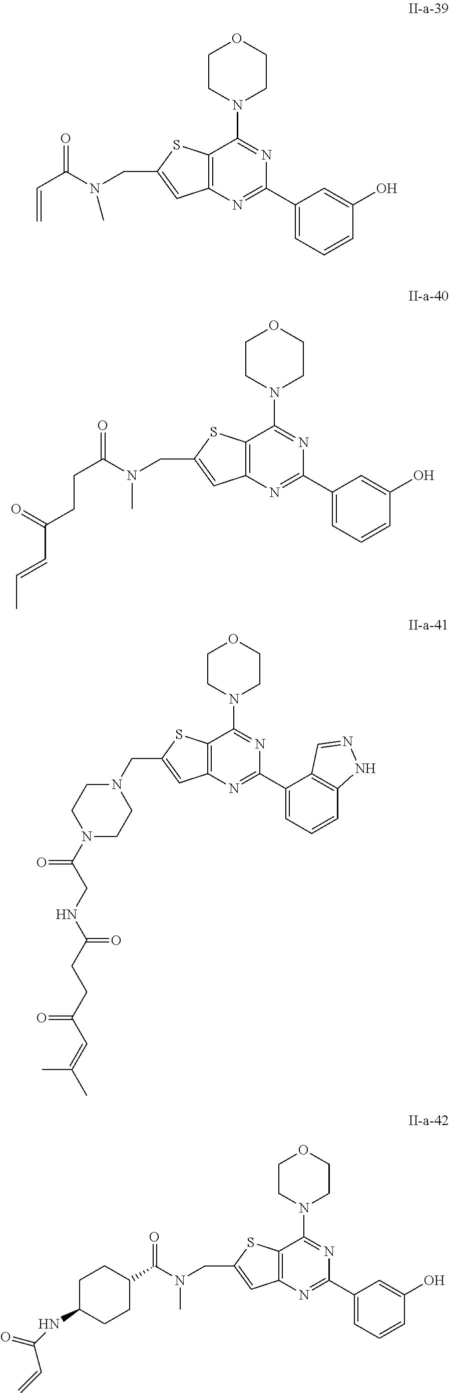

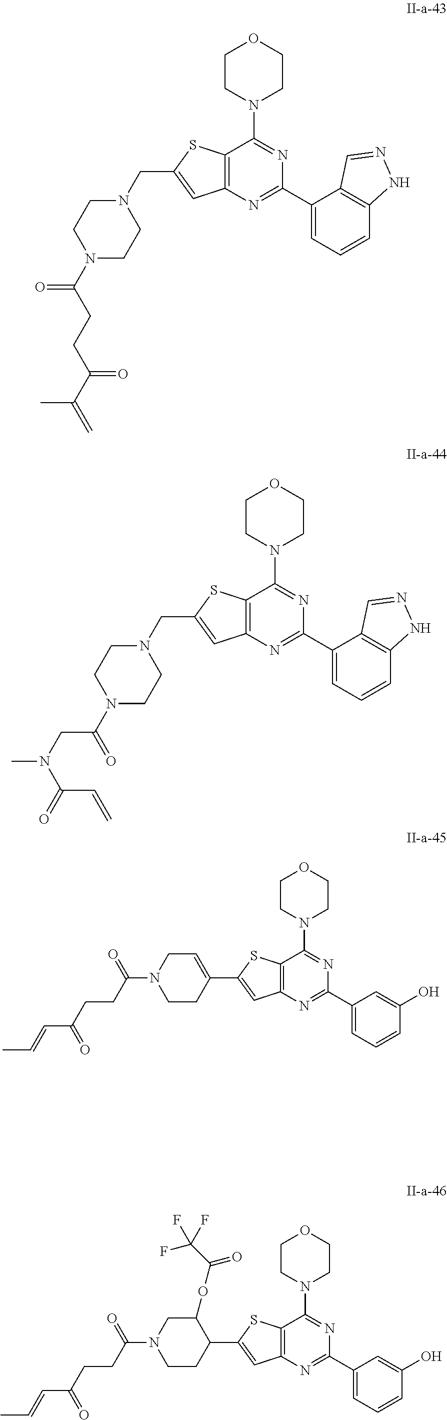

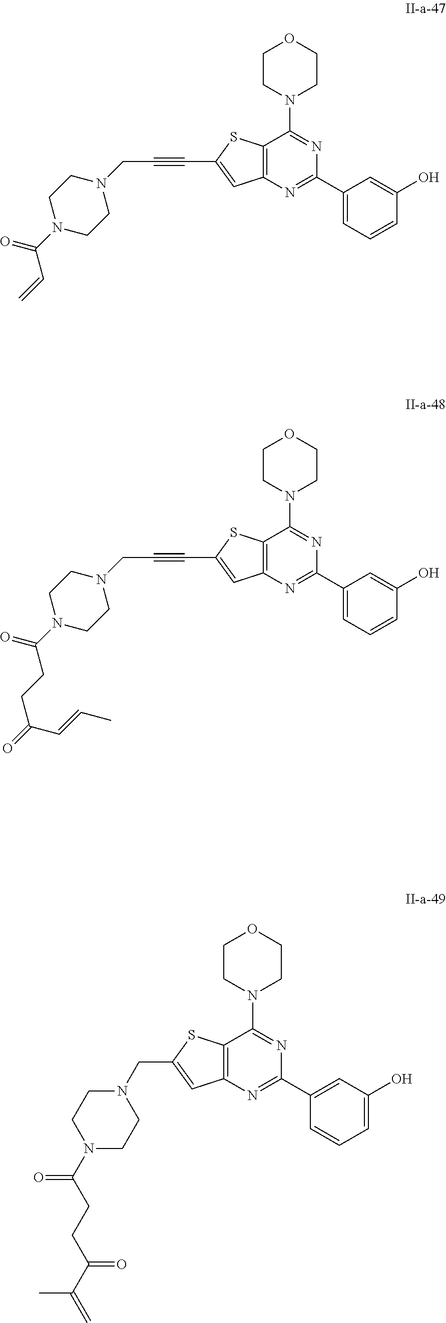

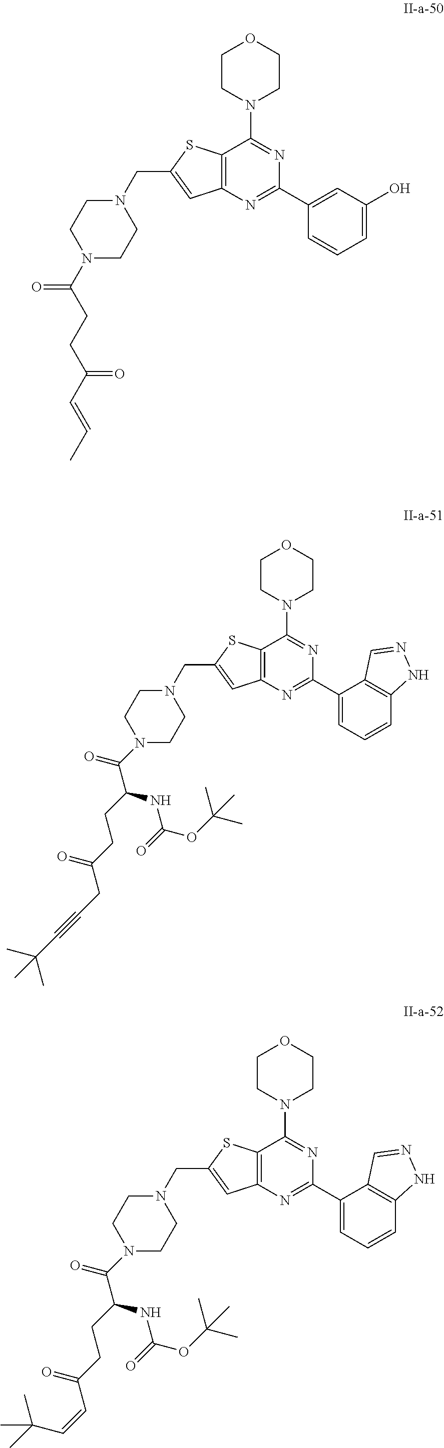

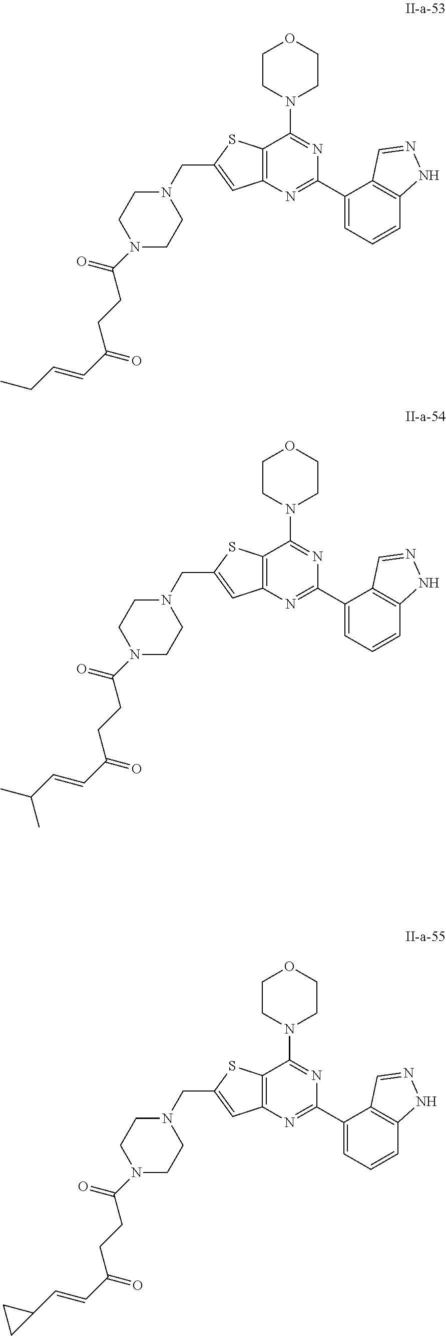

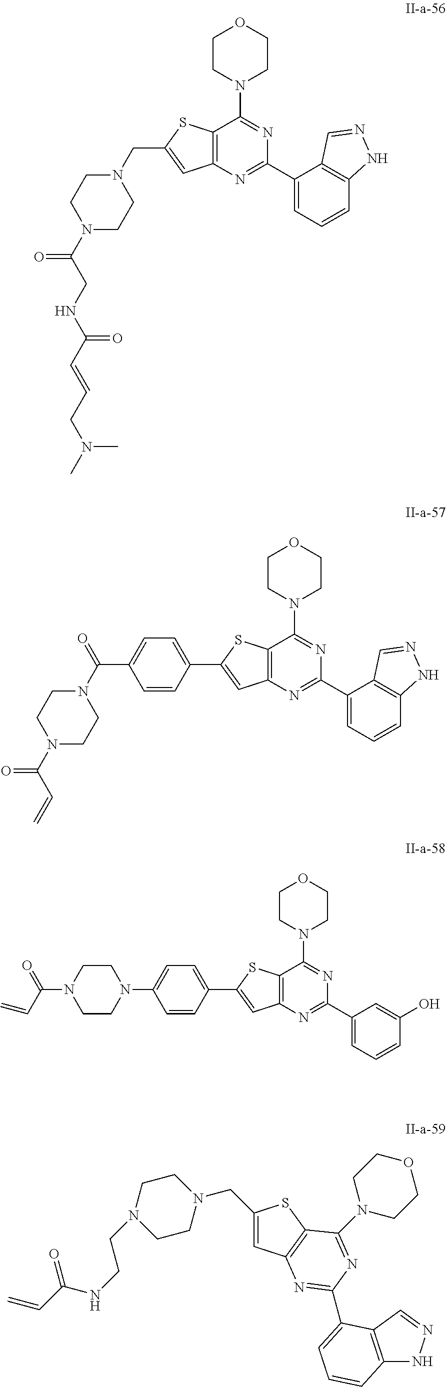

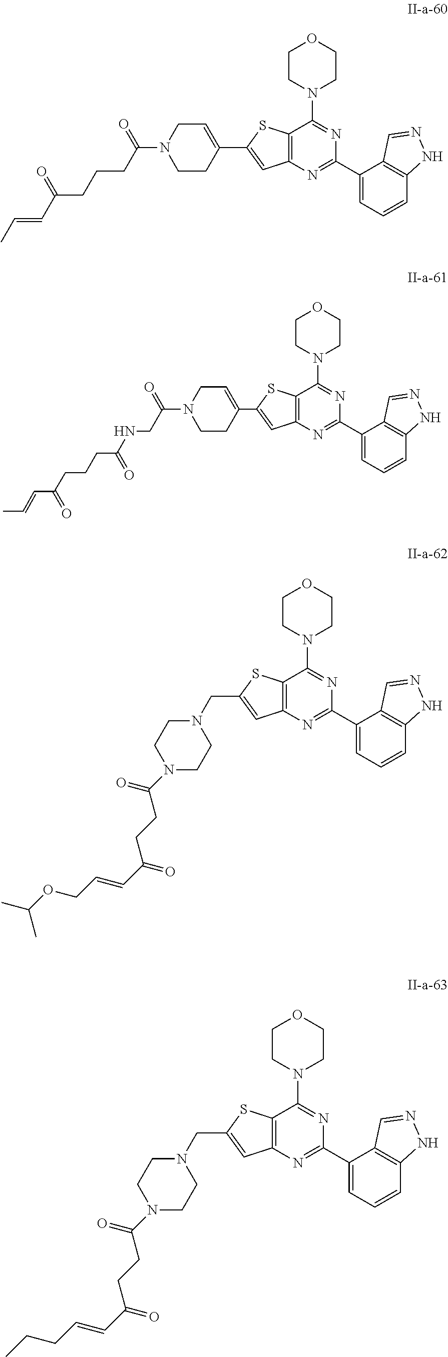

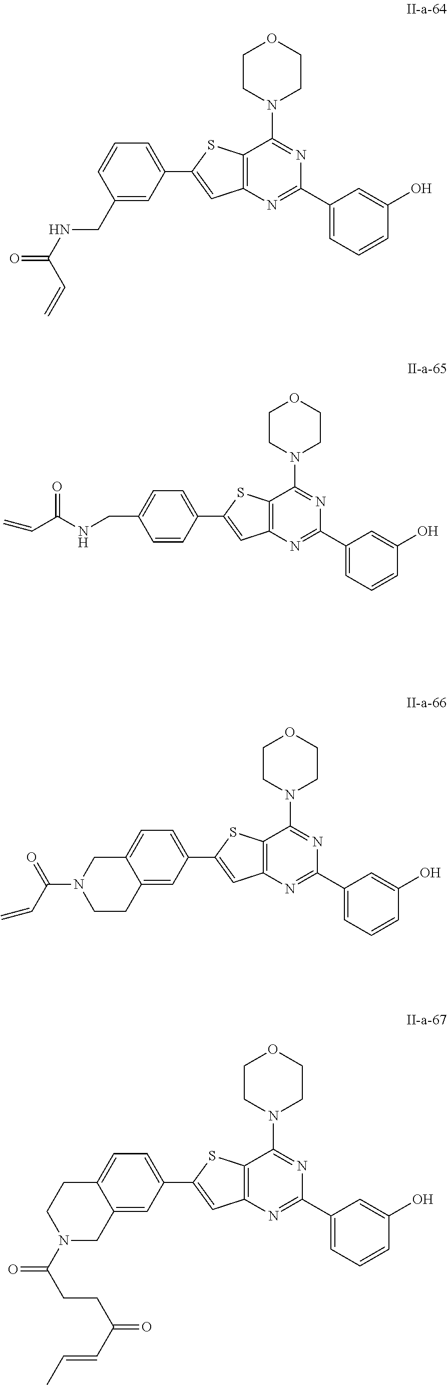

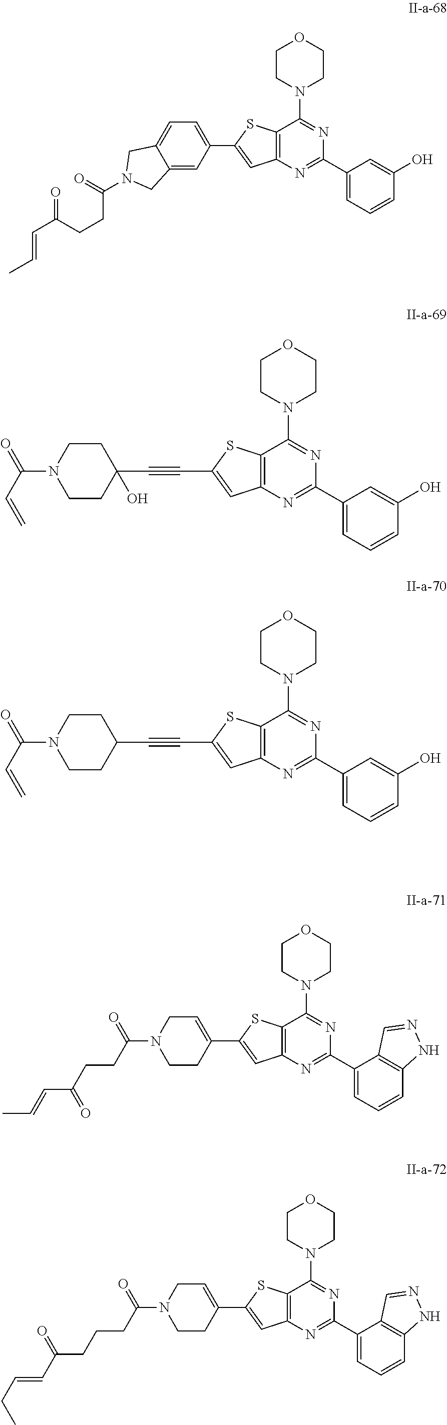

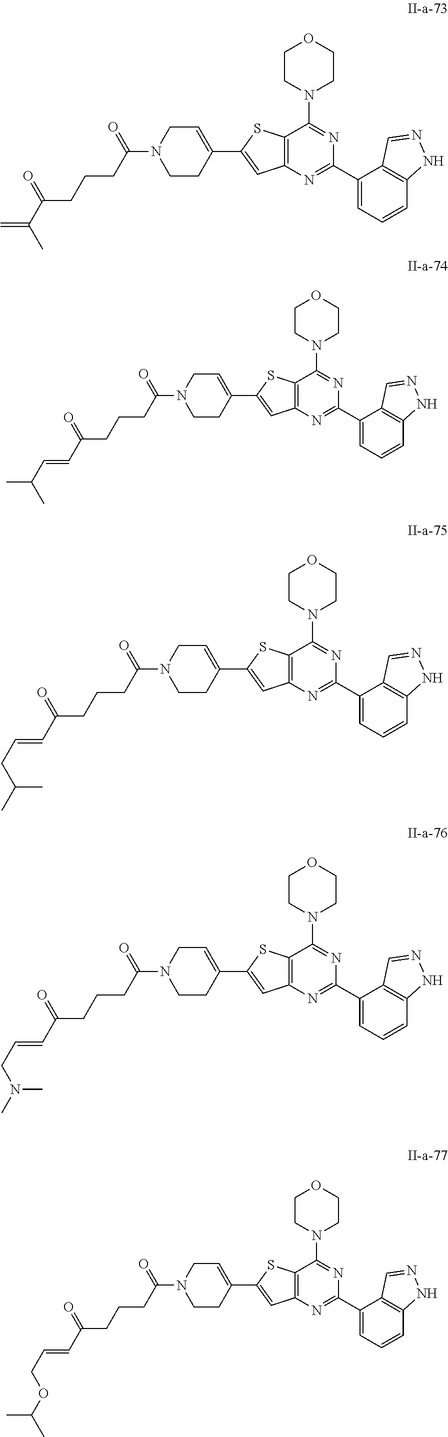

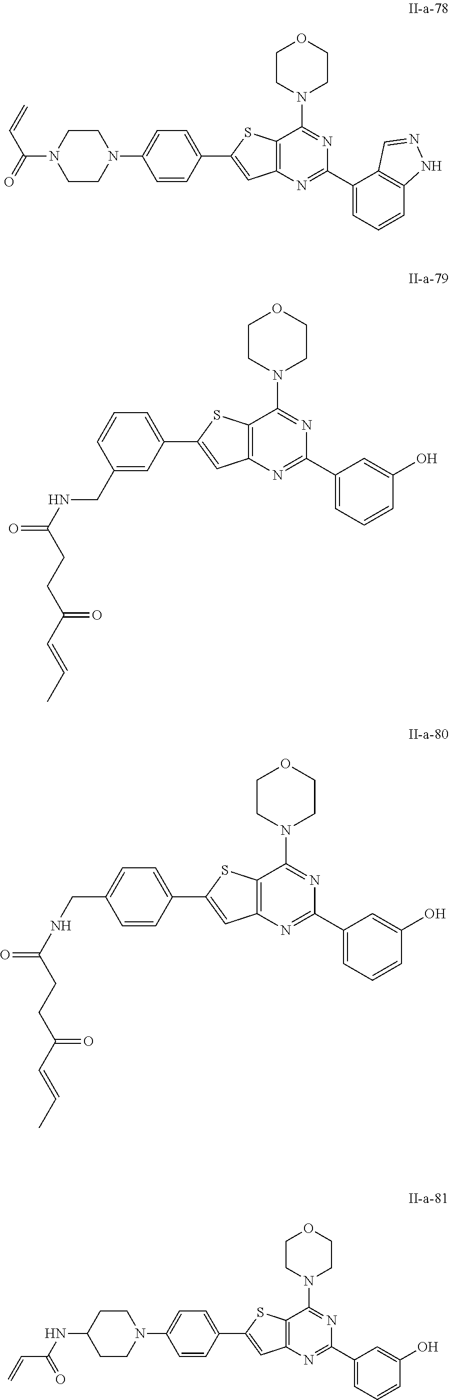

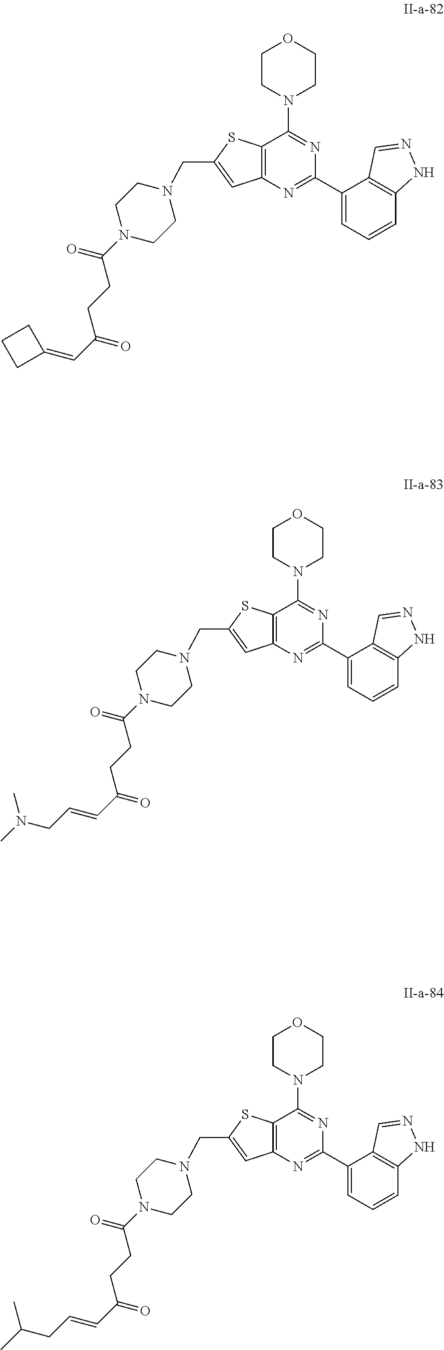

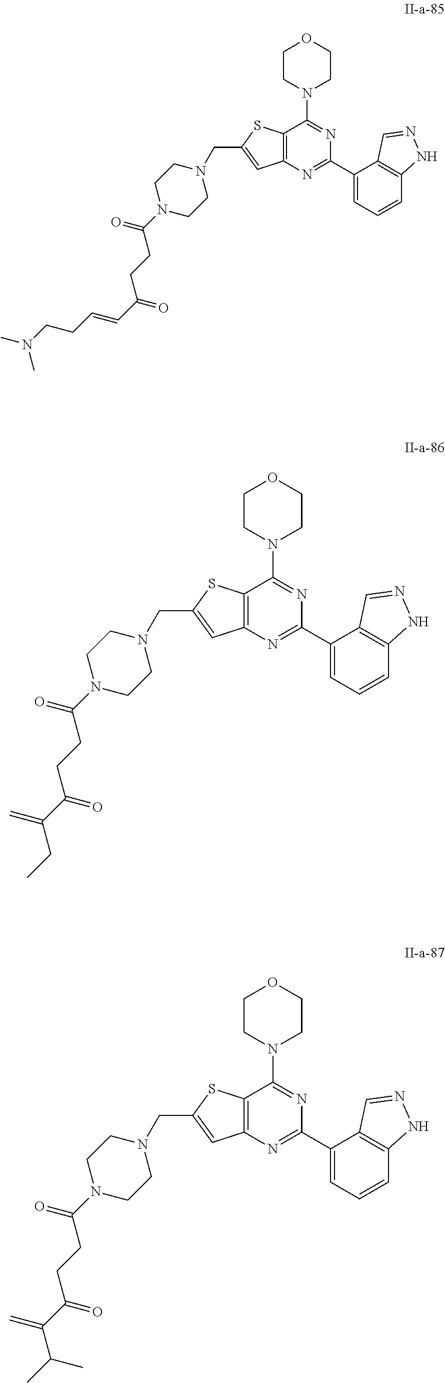

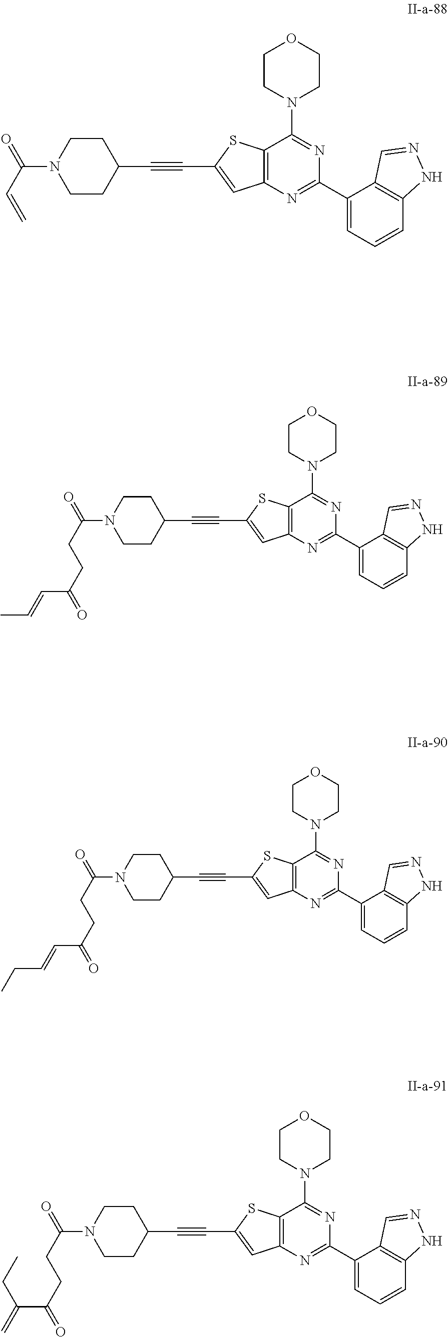

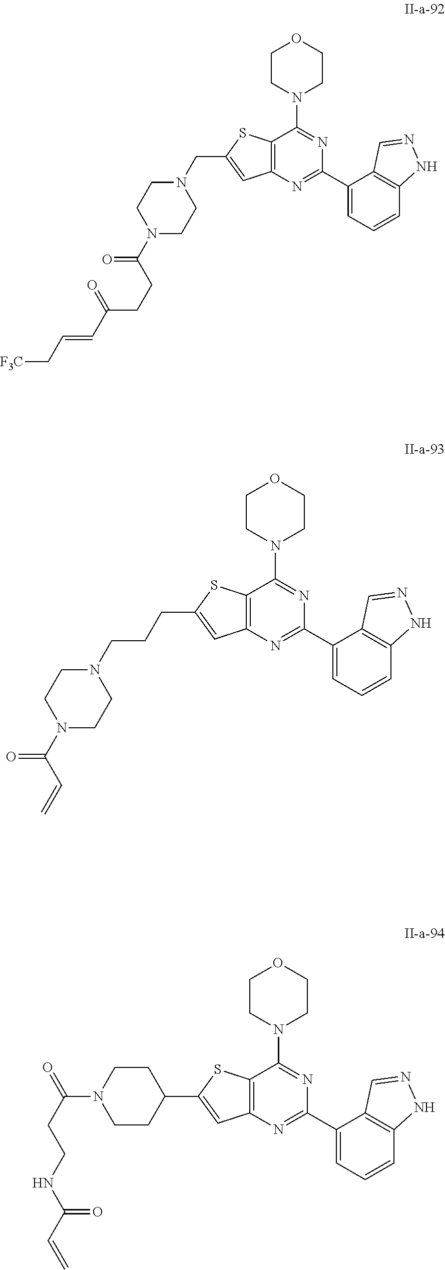

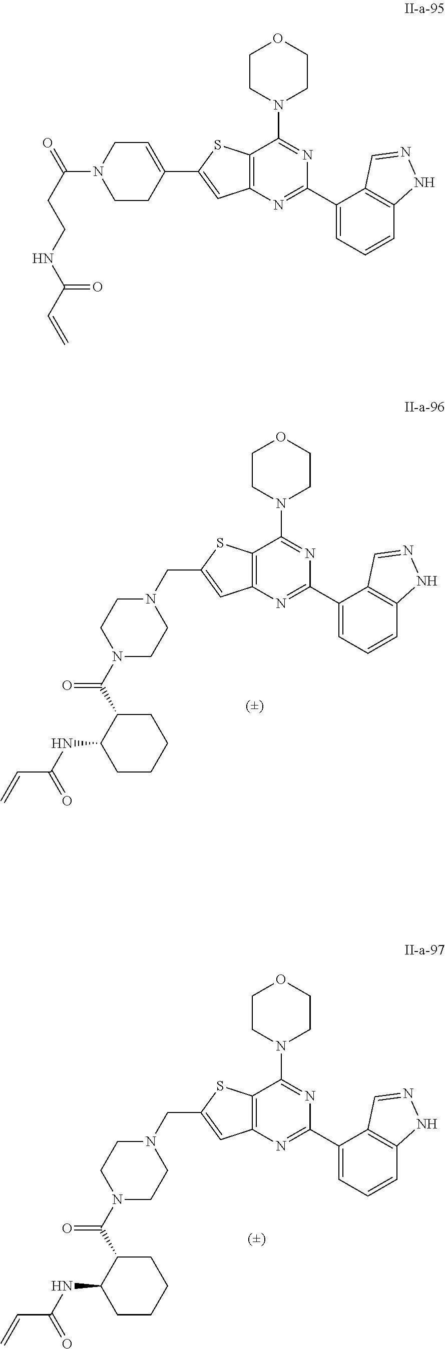

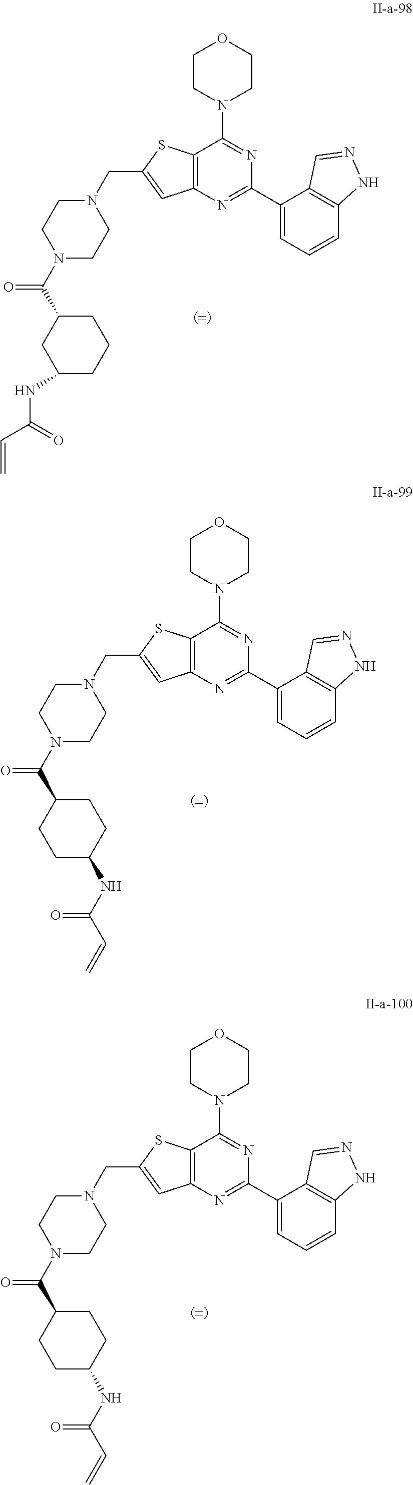

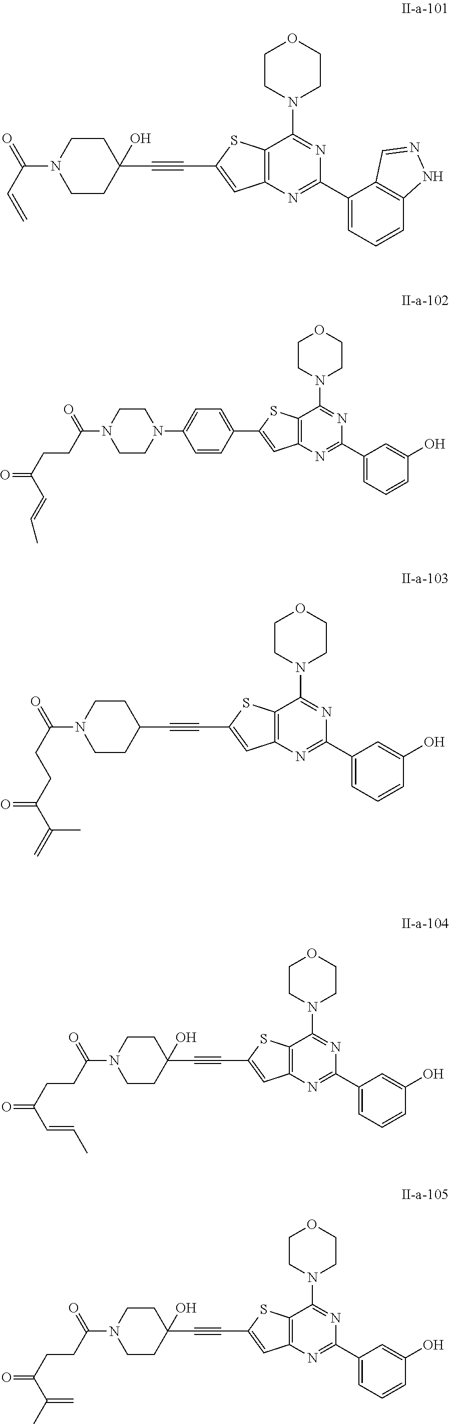

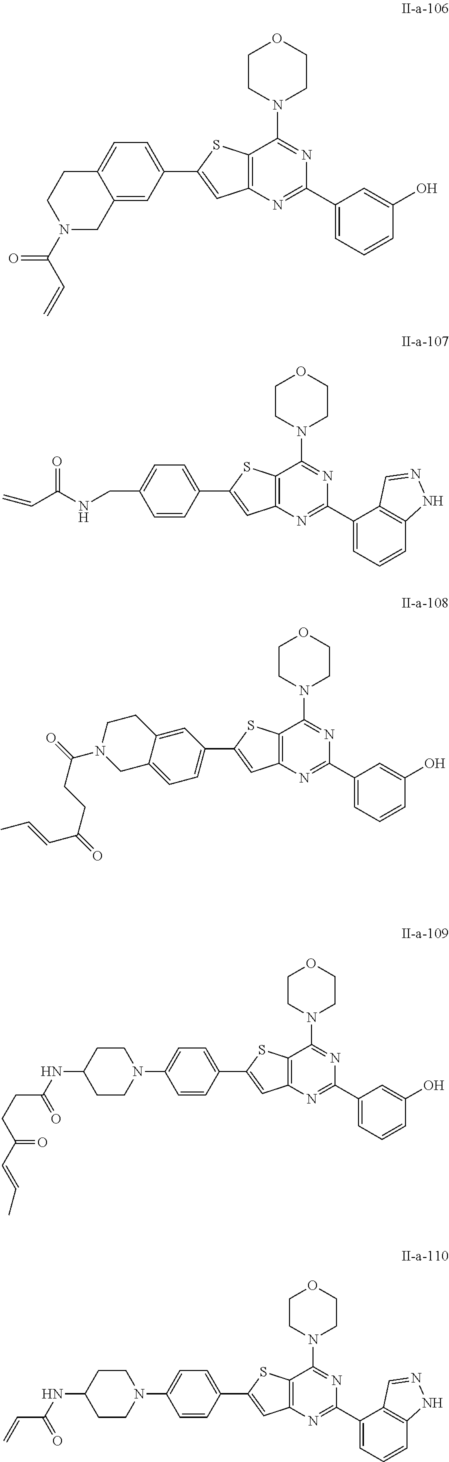

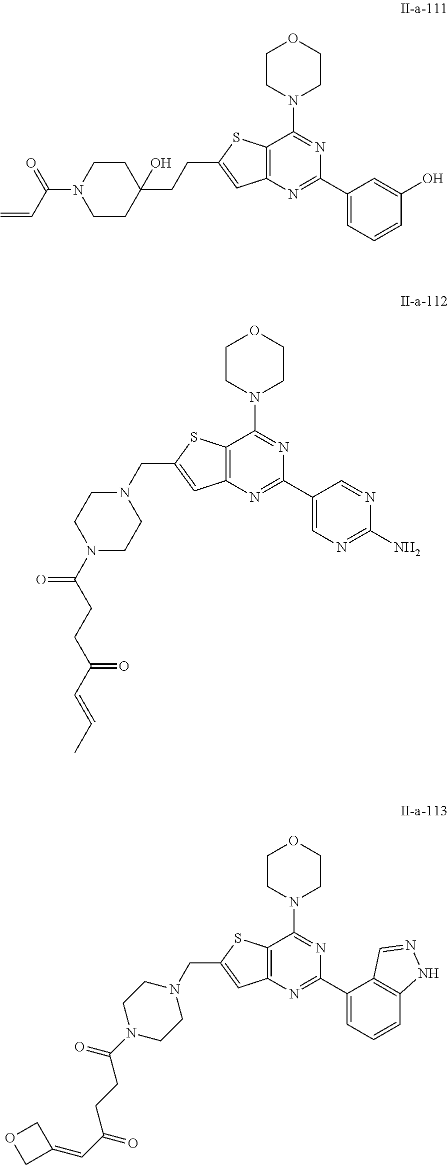

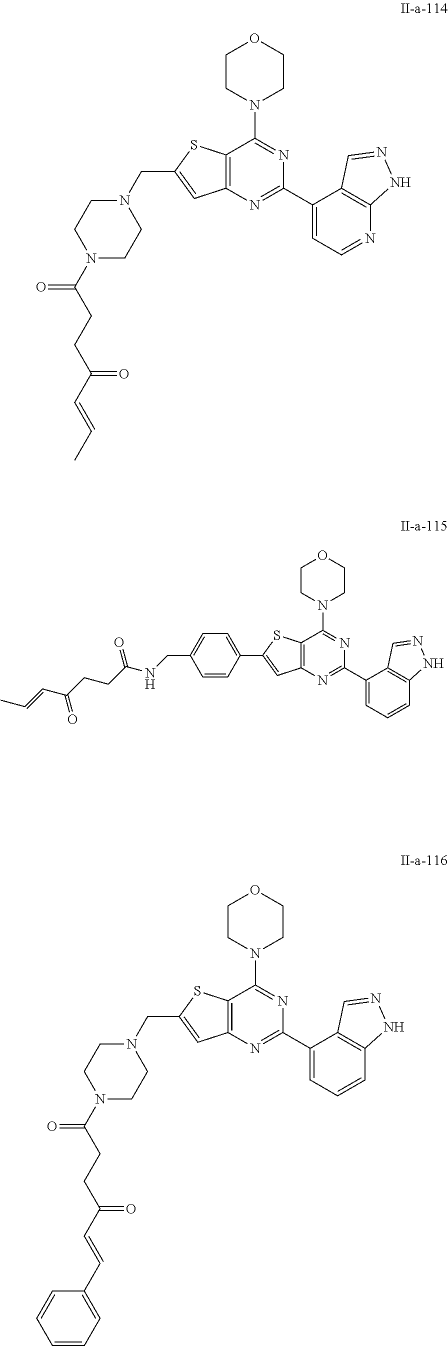

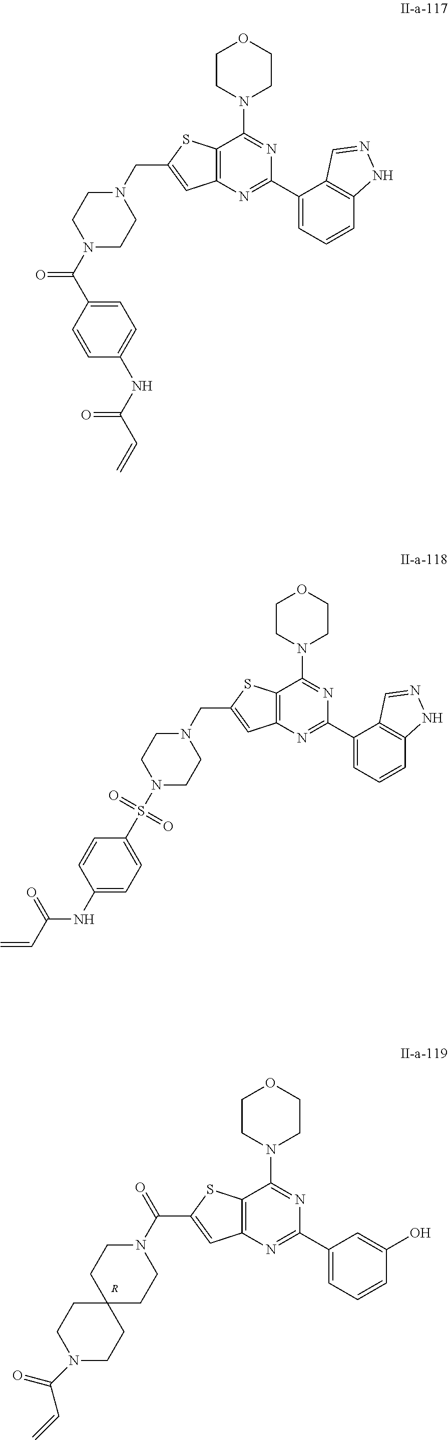

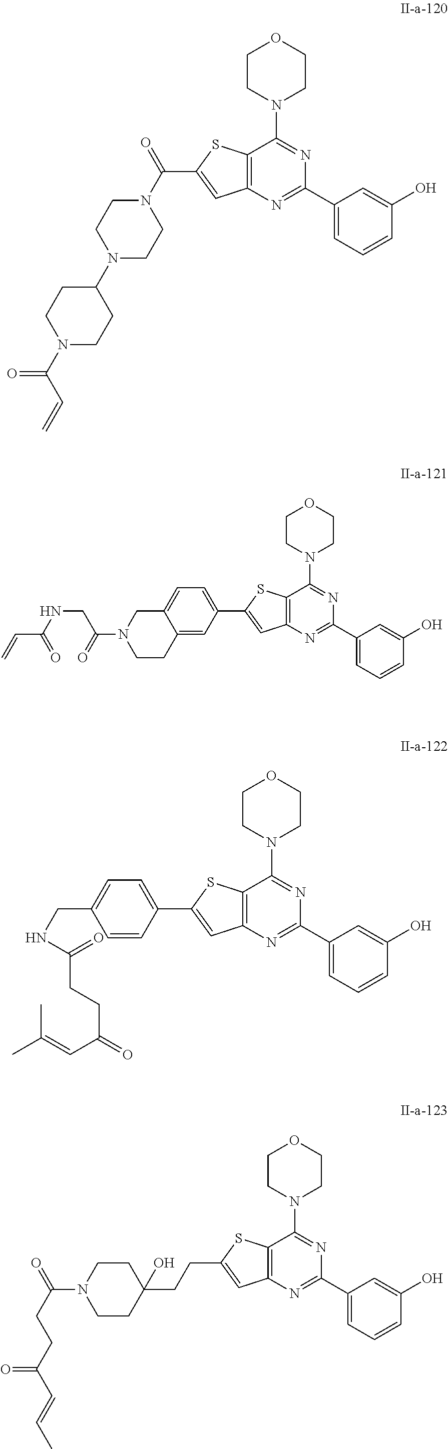

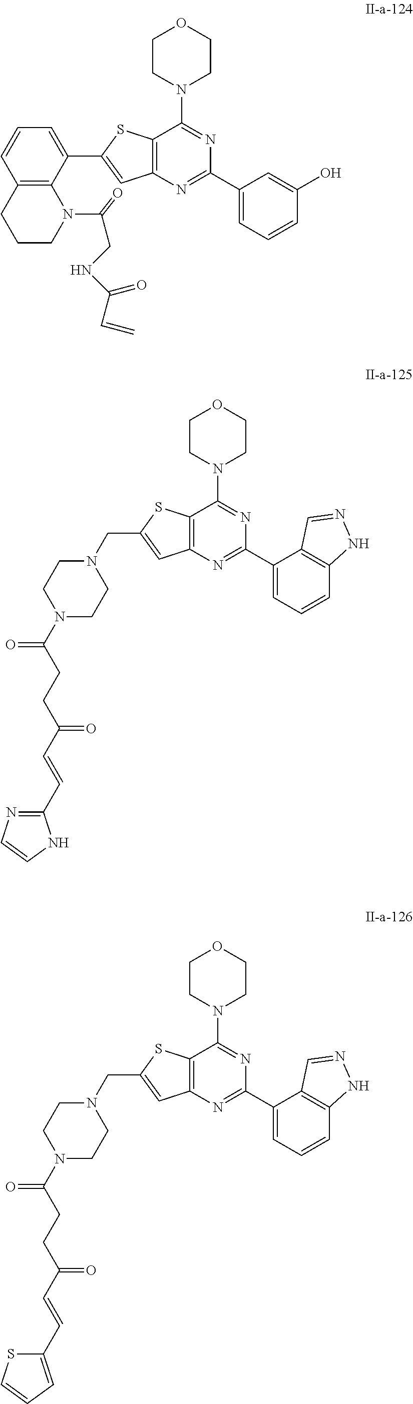

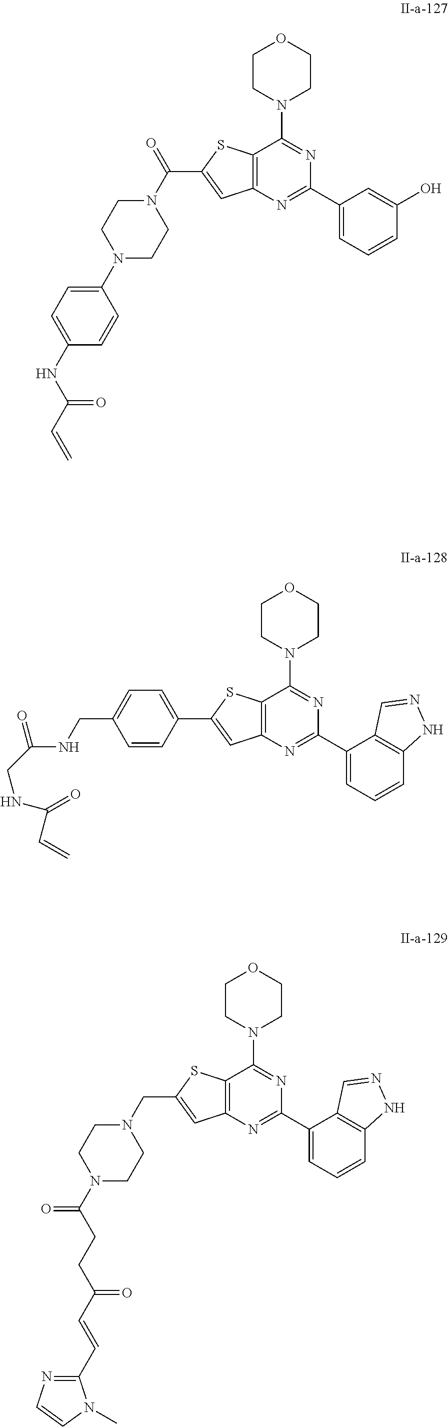

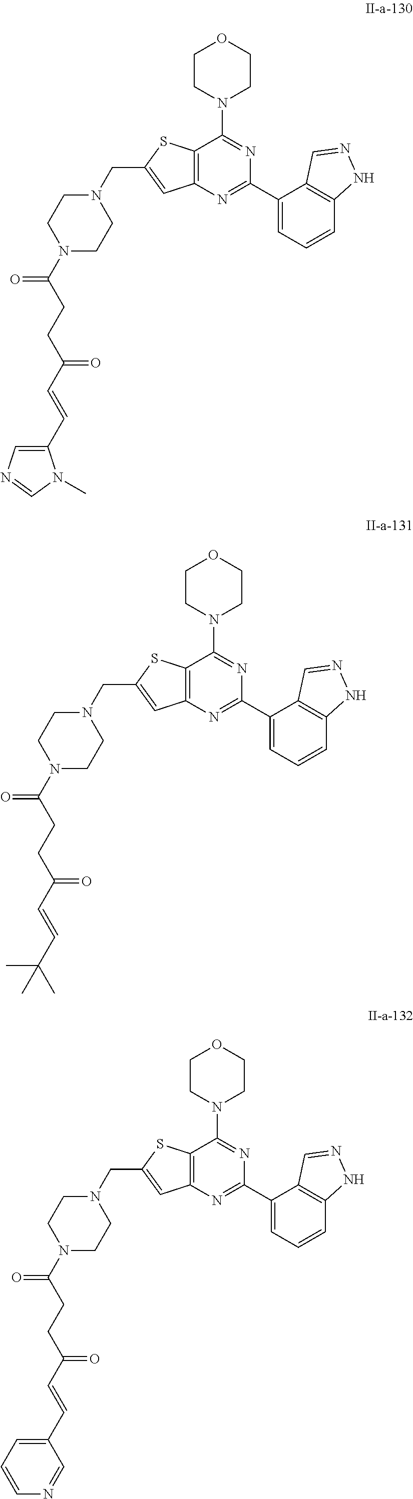

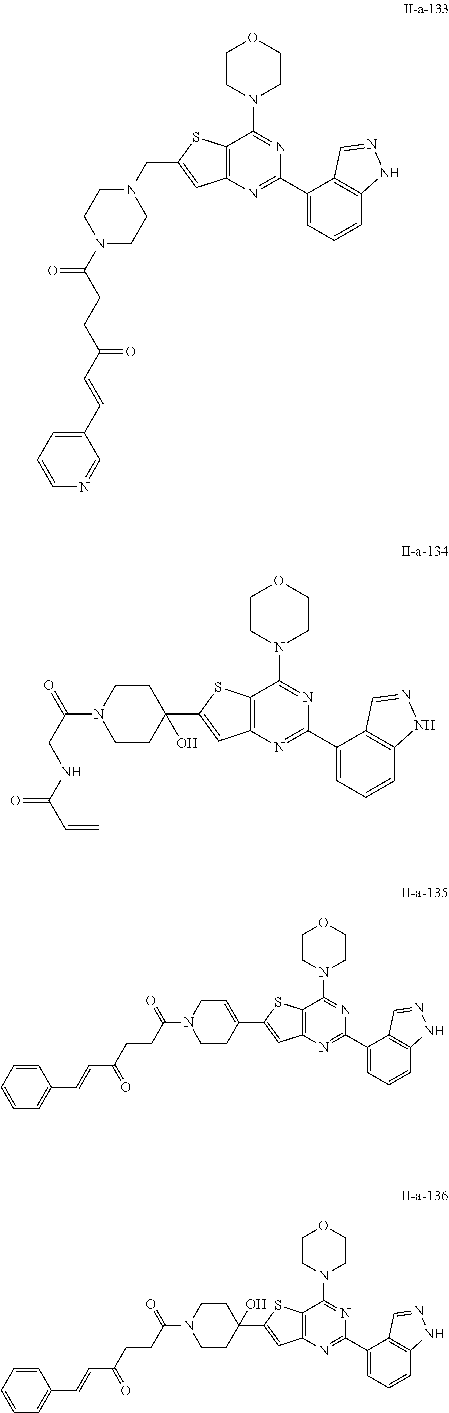

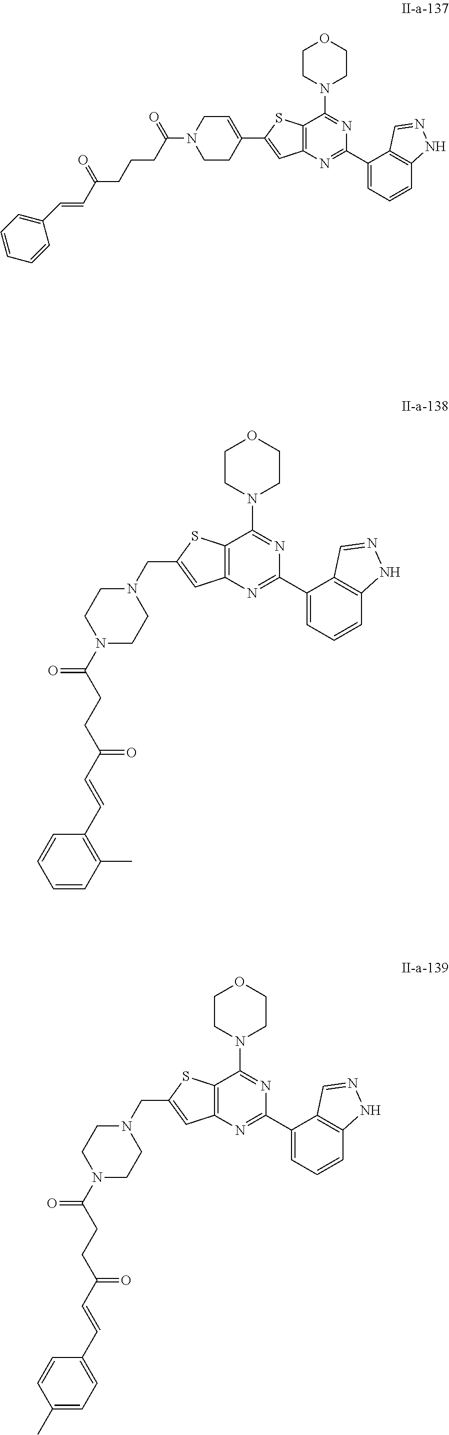

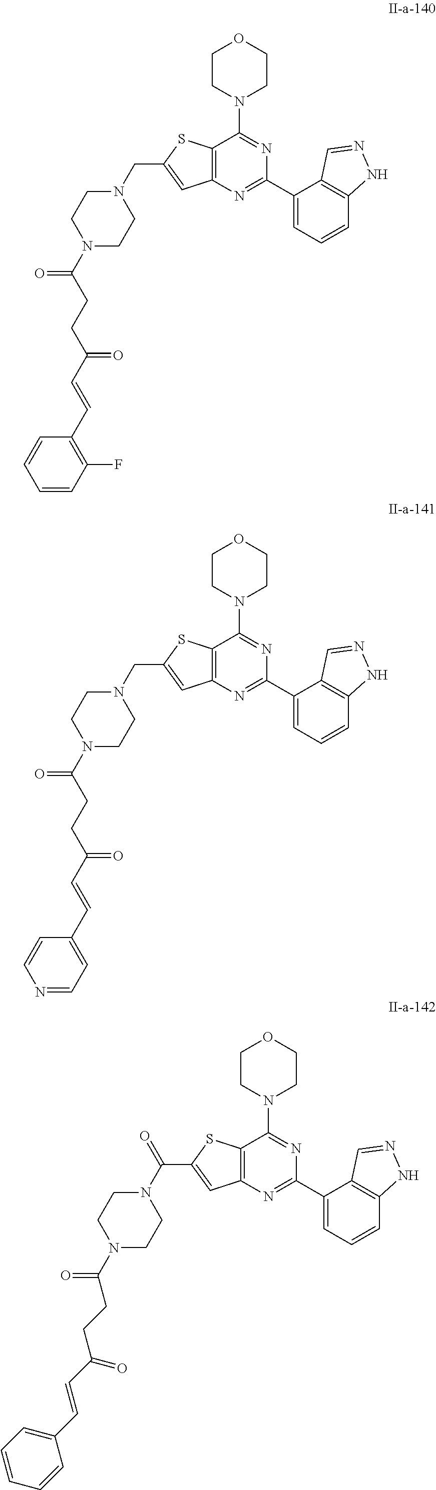

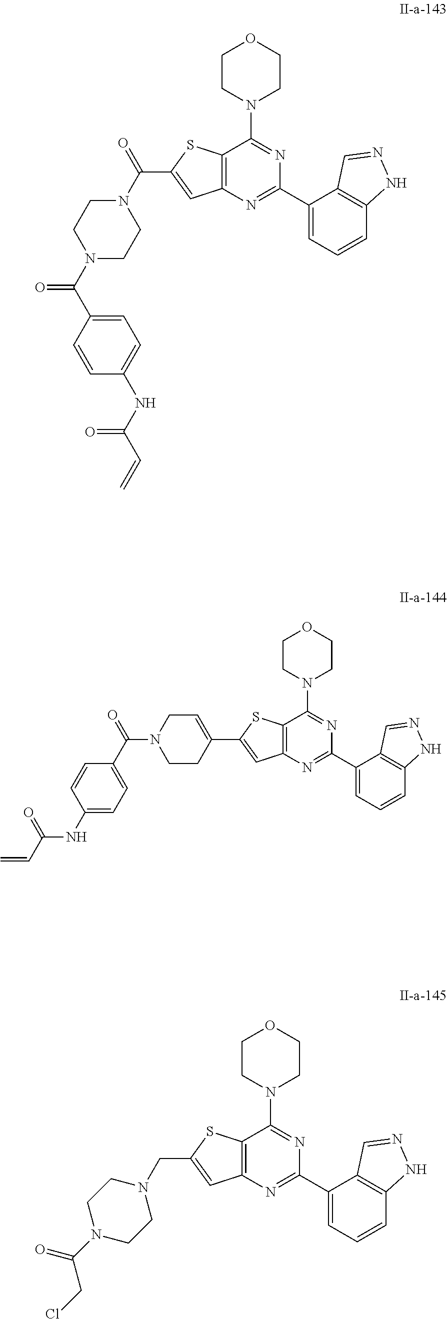

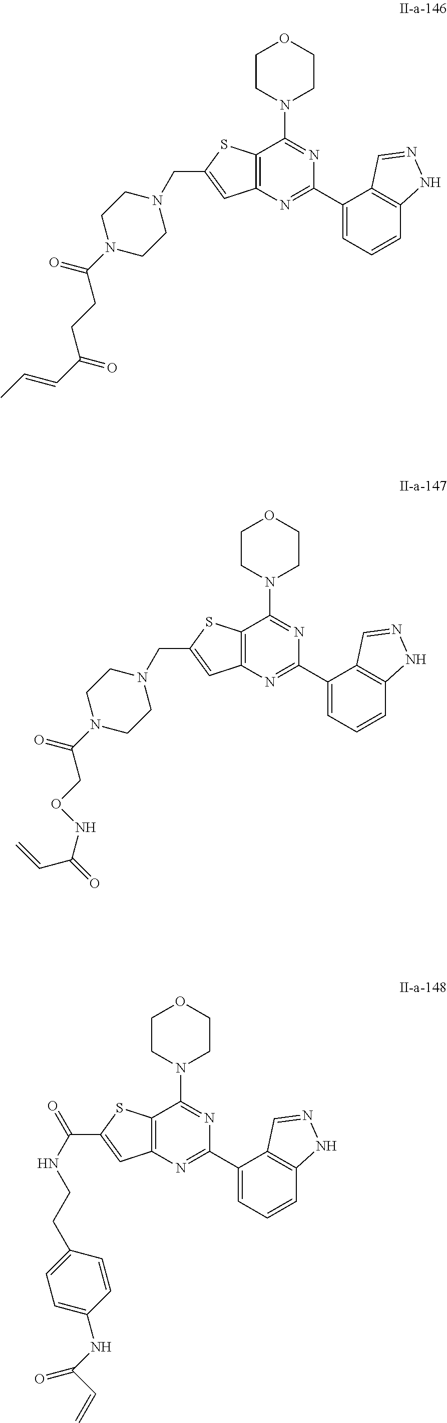

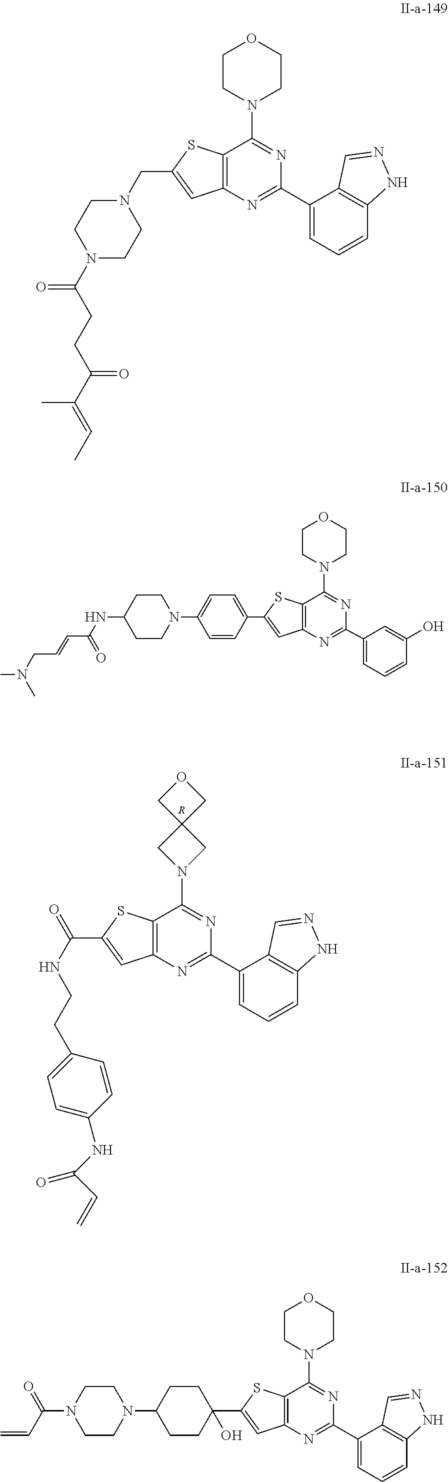

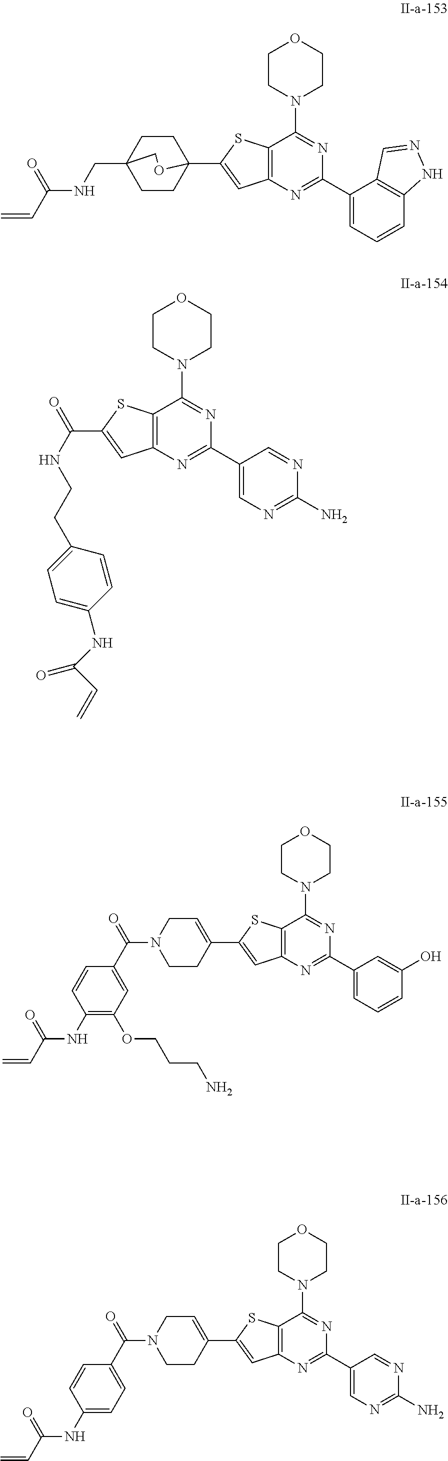

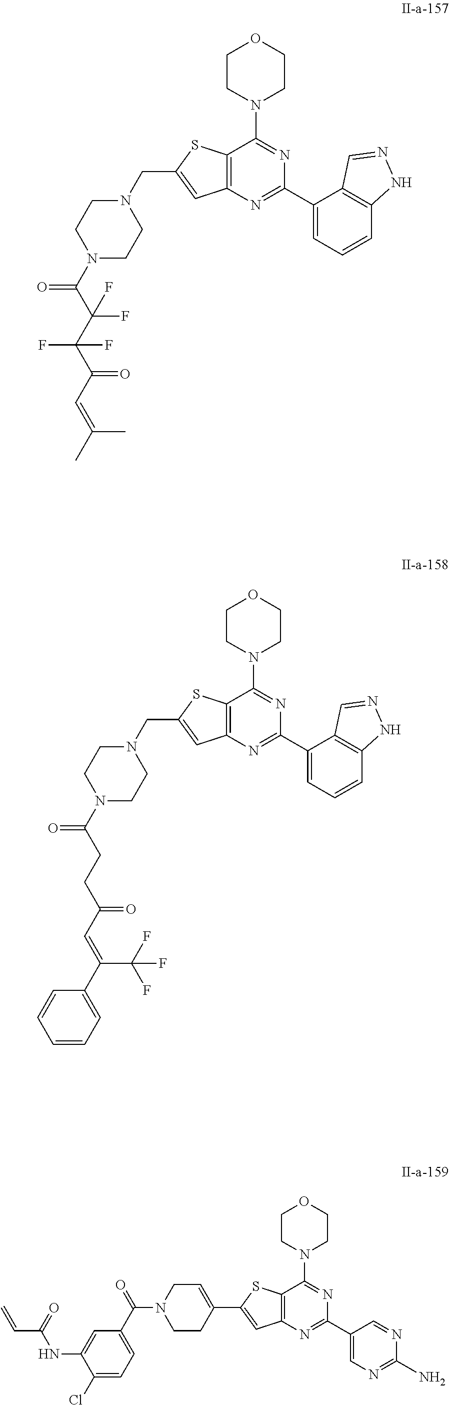

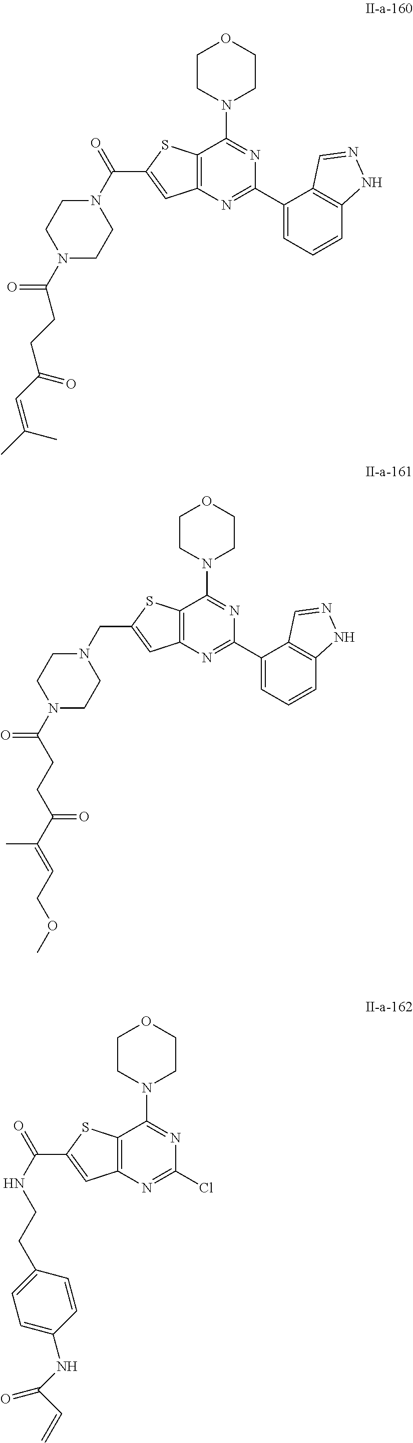

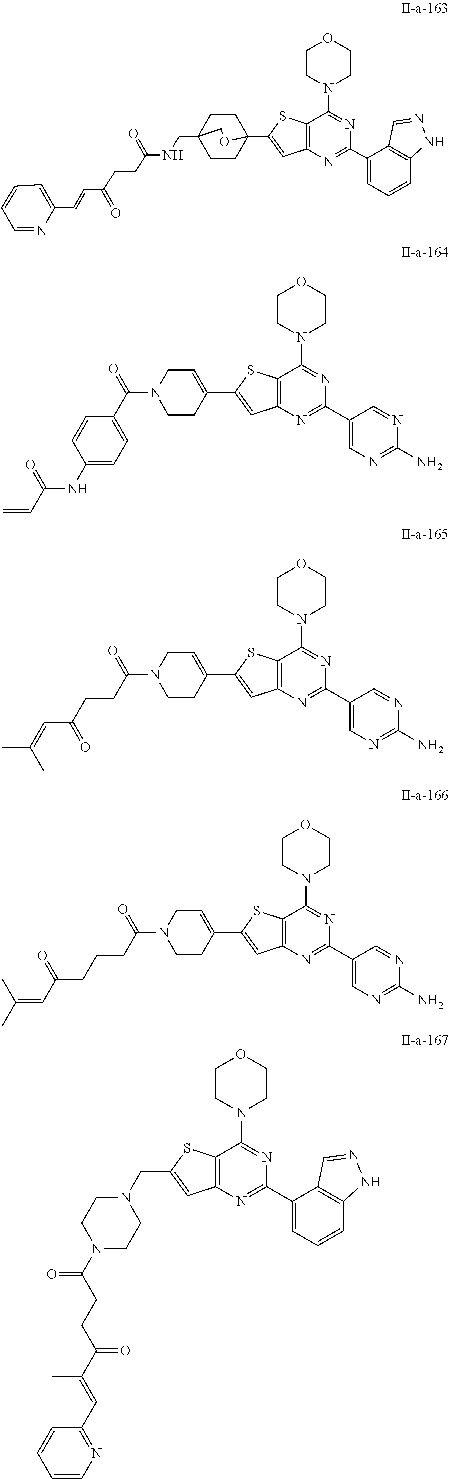

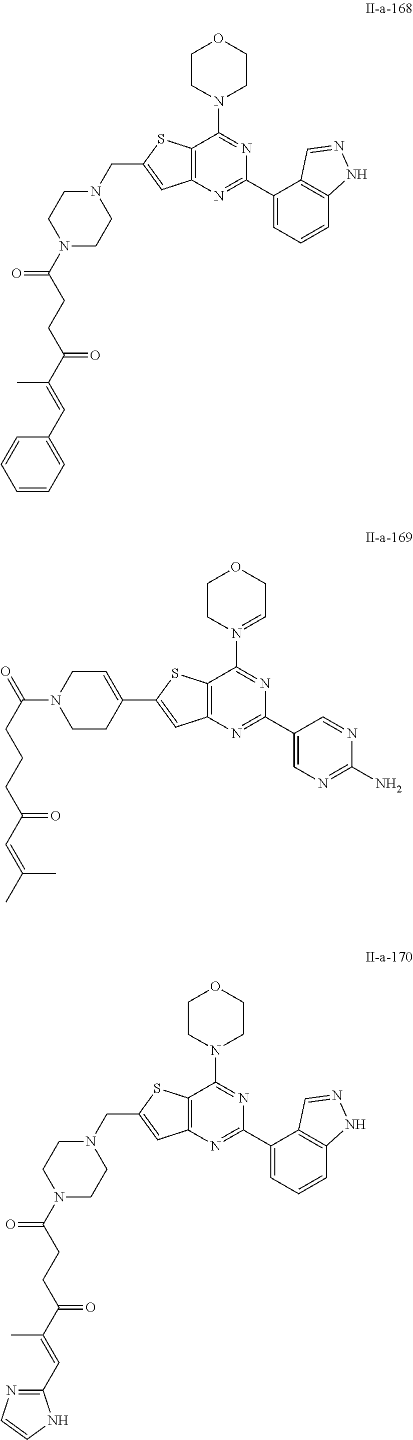

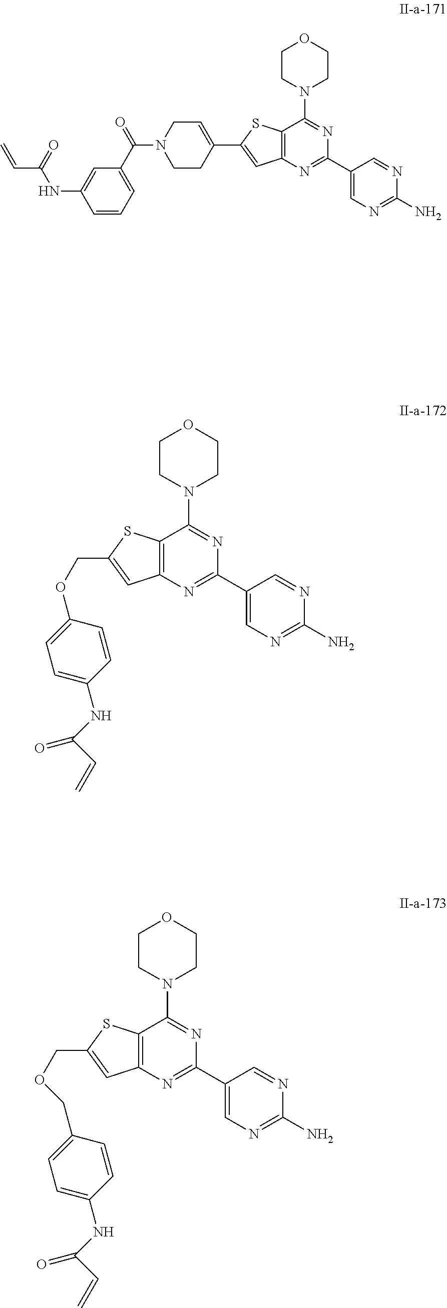

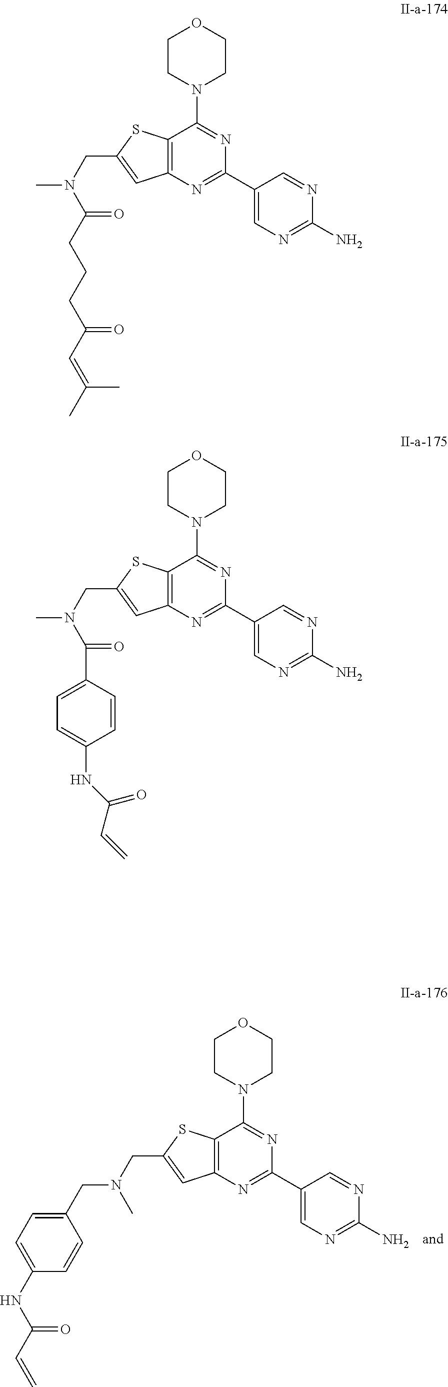

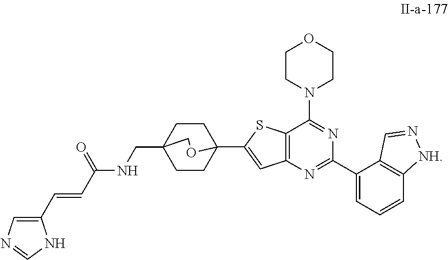

102. The compound according to claim 101 selected from the group consisting of:

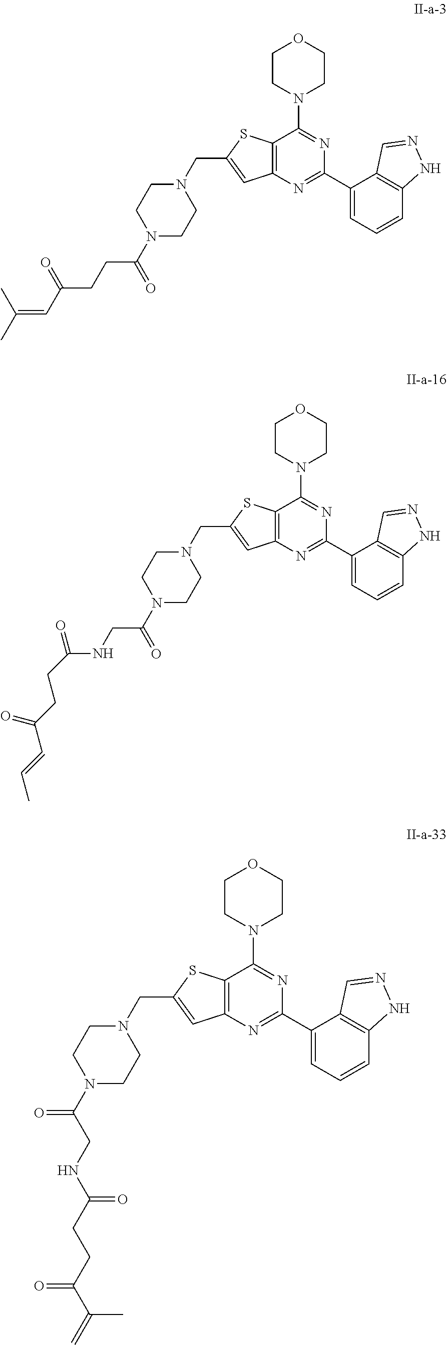

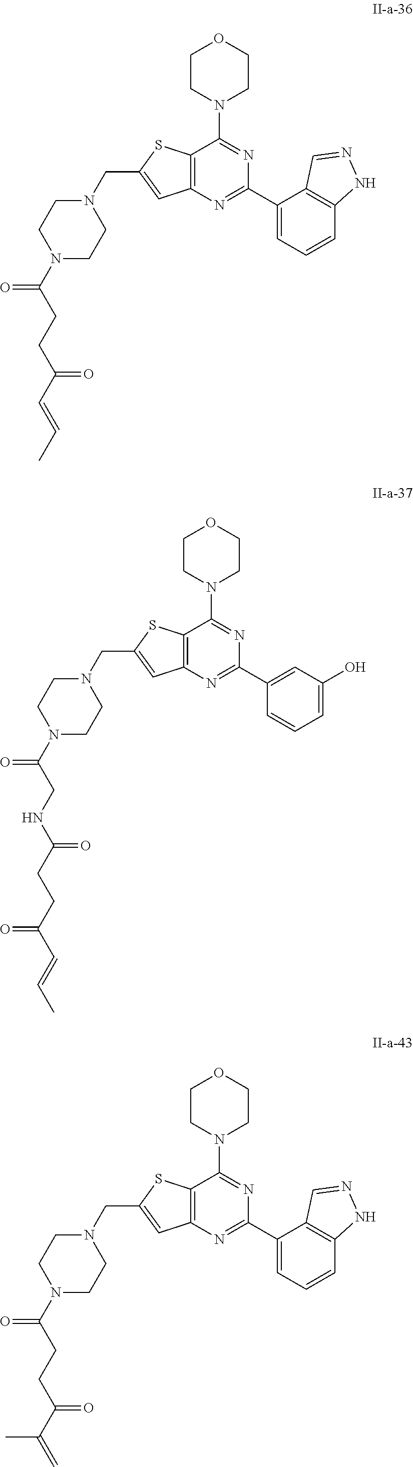

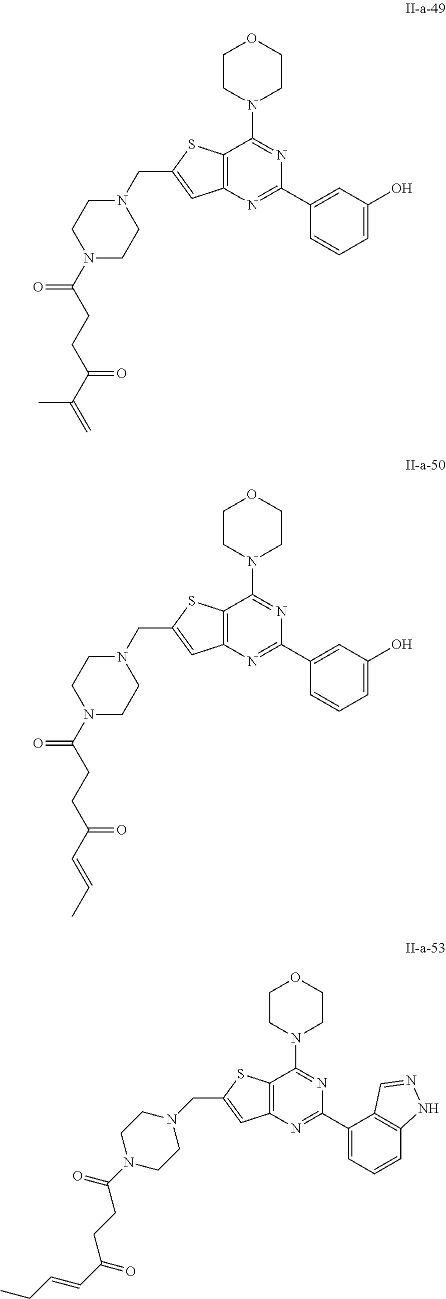

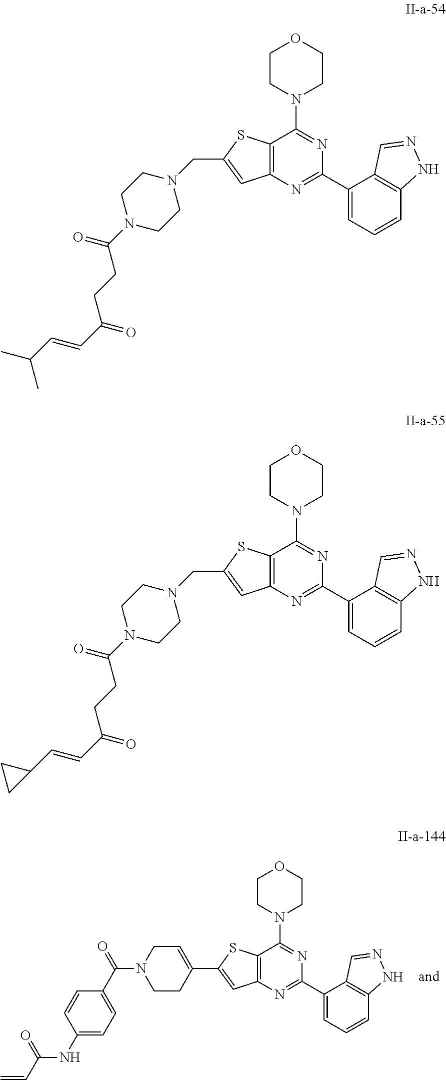

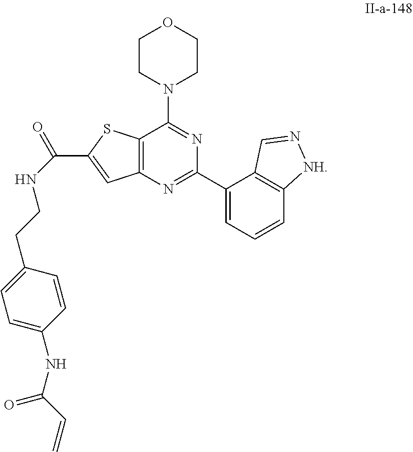

103. A compound of formula II-c or II-d: 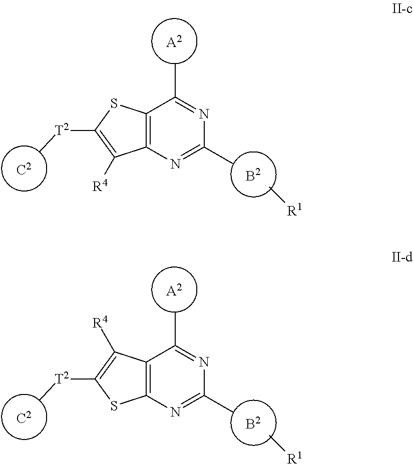
or a pharmaceutically acceptable salt thereof, wherein:
R¹ is a warhead group;
Ring A² is an optionally substituted ring selected from a 4-8 membered saturated or partially unsaturated heterocyclic ring having one or two heteroatoms independently selected from nitrogen, oxygen, or sulfur, or a 5-10 membered saturated or partially unsaturated bridged bicyclic heterocyclic ring having at least one nitrogen, at least one oxygen, and optionally 1-2 additional heteroatoms independently selected from nitrogen, oxygen, or sulfur;
R⁴ is R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B² is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T² is a covalent bond or a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring C² is hydrogen or an optionally substituted ring selected from a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

104-133. (canceled)

134. A compound of formula II-e or II-f: 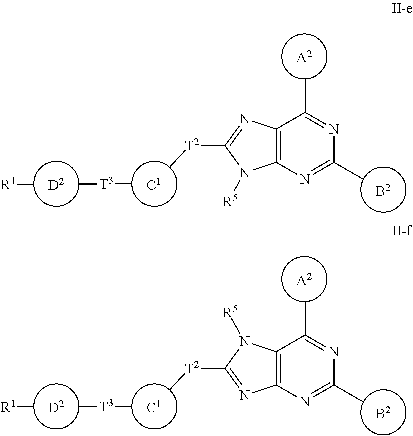
or a pharmaceutically acceptable salt thereof, wherein:
R¹ is a warhead group;
Ring A² is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R⁵ is R, —SO₂R, —SOR, —C(O)R, —CO₂R, or —C(O)N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B² is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T² is a covalent bond or a bivalent straight or branched, saturated or unsaturated C₁₋₆ hydrocarbon chain wherein one or more methylene units of T² are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C¹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T³ is a covalent bond or a bivalent straight or branched, saturated or unsaturated C₁₋₆ hydrocarbon chain wherein one or more methylene units of T³ are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring D² is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

135-176. (canceled)

177. A compound of formula II-g or II-h: 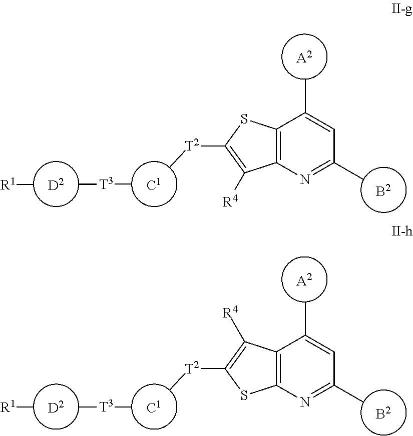
or a pharmaceutically acceptable salt thereof, wherein:
R¹ is a warhead group;
Ring A² is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R⁴ is —R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B² is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T² is a covalent bond or a bivalent straight or branched, saturated or unsaturated C₁₋₆ hydrocarbon chain wherein one or more methylene units of T² are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C¹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T³ is a covalent bond or a bivalent straight or branched, saturated or unsaturated C₁₋₆ hydrocarbon chain wherein one or more methylene units of T³ are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring D² is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

178-226. (canceled)

227. A compound of formula III: 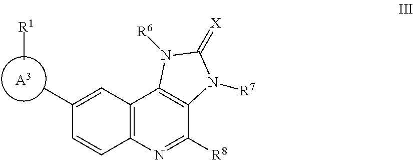
or a pharmaceutically acceptable salt thereof, wherein:
R¹ is a warhead group;
X is O or S;
R⁶ is an optionally substituted group selected from phenyl, napthyl, a 6-membered heteroaryl ring having 1-2 nitrogens, or an 8-10 membered bicyclic heteroaryl ring having 1-3 heteroatoms independently selected from nitrogen, oxygen, or sulfur;
R⁷ is an optionally substituted C₁₋₆ aliphatic group;
R⁸ is hydrogen or —NHR′;
R′ is independently hydrogen or an optionally substituted C₁₋₆ aliphatic group; and
Ring A³ is an optionally substituted group selected from phenyl, naphthyl, a 6-membered heteroaryl ring having 1-2 nitrogens, or an 8-10 membered bicyclic heteroaryl ring having 1-3 nitrogens.

228-234. (canceled)

235. A compound of formula IV: 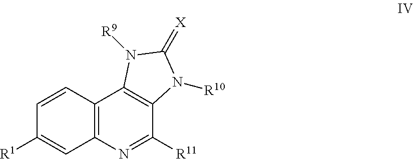
or a pharmaceutically acceptable salt thereof, wherein:
R¹ is a warhead group;
X is O or S;
R⁹ is an optionally substituted group selected from phenyl, napthyl, a 6-membered heteroaryl ring having 1-2 nitrogens, or an 8-10 membered bicyclic heteroaryl ring having 1-3 heteroatoms independently selected from nitrogen, oxygen, or sulfur;
R¹⁰ is an optionally substituted C₁₋₆ aliphatic group;
R¹¹ is hydrogen or —NHR′; and
R′ is independently hydrogen or an optionally substituted C₁₋₆ aliphatic group.

236-239. (canceled)

240. A compound of formula V-a or V-b: 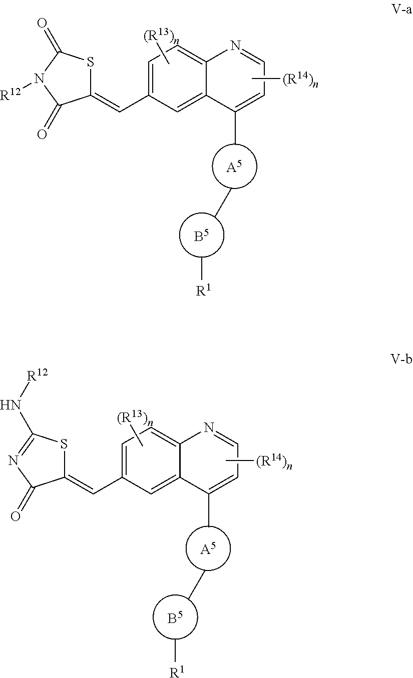
or a pharmaceutically acceptable salt thereof, wherein:
R¹ is a warhead group;
R¹² is an hydrogen or an optionally substituted group selected from C₁₋₆ aliphatic, —(CH₂)ₘ-(3-7 membered saturated or partially unsaturated carbocyclic ring), —(CH₂)ₘ-(7-10 membered saturated or partially unsaturated bicyclic carbocyclic ring), —(CH₂)ₘ-(4-7 membered saturated or partially unsaturated heterocyclic ring having 1-2 heteroatoms independently selected from nitrogen, oxygen, or sulfur), —(CH₂)ₘ-(7-10 membered saturated or partially unsaturated bicyclic heterocyclic ring having 1-3 heteroatoms independently selected from nitrogen, oxygen, or sulfur), —(CH₂)ₘ-phenyl, —(CH₂)ₘ-(8-10 membered bicyclic aryl ring), —(CH₂)ₘ-(5-6 membered heteroaryl ring having 1-3 heteroatoms independently selected from nitrogen, oxygen, or sulfur), or —(CH₂)ₘ-(8-10 membered bicyclic heteroaryl ring having 1-4 heteroatoms independently selected from nitrogen, oxygen, or sulfur);
each R¹³ and R¹⁴ is independently —R″, halogen, —NO₂, —CN, —OR″, —SR″, —N(R″)₂, —C(O)R″, —CO₂R″, —C(O)C(O)R″, —C(O)CH₂C(O)R″, —S(O)R″, —S(O)₂R″, —C(O)N(R″)₂, —SO₂N(R″)₂, —OC(O)R″, —N(R″)C(O)R″, —N(R″)N(R″)₂, —N(R″)C(═NR″)N(R″)₂, —C(═NR″)N(R″)₂, —C═NOR″, —N(R″)C(O)N(R″)₂, —N(R″)SO₂N(R″)₂, —N(R″)SO₂R″, or —OC(O)N(R″)₂;
each R″ is independently hydrogen or an optionally substituted group selected from C₁₋₆ aliphatic, a 3-7 membered saturated or partially unsaturated carbocyclic ring, a 7-10 membered saturated or partially unsaturated bicyclic carbocyclic ring, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; or
two R″ groups on the same nitrogen are taken together with the nitrogen to which they are attached to form an optionally substituted 5-8 membered saturated, partially unsaturated, or aromatic ring having 1-4 heteroatoms independently selected from nitrogen, oxygen, or sulfur;
m is an integer from 0 to 6, inclusive;
each n is independently 0, 1, or 2;
Ring A⁵ is an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; and
Ring B⁵ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

241-251. (canceled)

252. A compound of formula VI-a or VI-b: 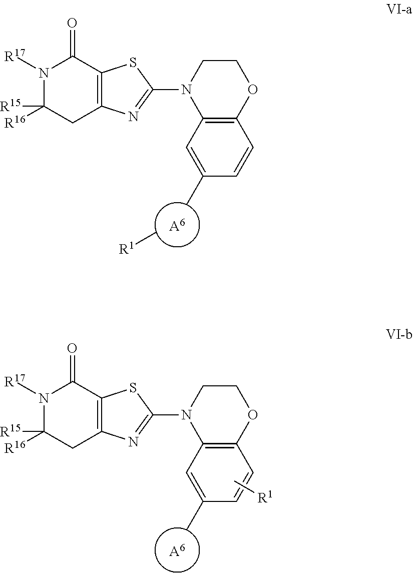
or a pharmaceutically acceptable salt thereof, wherein:
R¹ is a warhead group;
R¹⁵ is hydrogen or C₁₋₆ alkyl;
R¹⁶ is hydrogen or an optionally substituted group selected from C₁₋₆ alkyl, C₁₋₆ alkoxy, or (C₁₋₆ alkylene)-R¹⁸; or
R¹⁵ and R¹⁶ are taken together with the intervening carbon to form an optionally substituted ring selected from a 3-7 membered carbocyclic ring or a 4-7 membered heterocyclic ring having 1-2 heteroatoms independently selected from nitrogen, oxygen, or sulfur;
R¹⁷ is hydrogen or C₁₋₆ alkyl;
R¹⁸ is a 3-7 membered saturated or partially unsaturated carbocyclic ring, a 7-10 membered saturated or partially unsaturated bicyclic carbocyclic ring, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, a 8-10 membered bicyclic aryl ring, a 5-6 membered heteroaryl ring having 1-3 heteroatoms independently selected from nitrogen, oxygen, or sulfur, or a 8-10 membered bicyclic heteroaryl ring having 1-4 heteroatoms independently selected from nitrogen, oxygen, or sulfur; and
Ring A⁶ is absent or an optionally substituted group selected from a 4-7 membered heterocyclic ring having 1-2 heteroatoms independently selected from nitrogen, oxygen, or sulfur, or a 5-6 membered heteroaryl ring having 1-3 heteroatoms independently selected from nitrogen, oxygen, or sulfur.

253-258. (canceled)

259. A compound of formula VII: 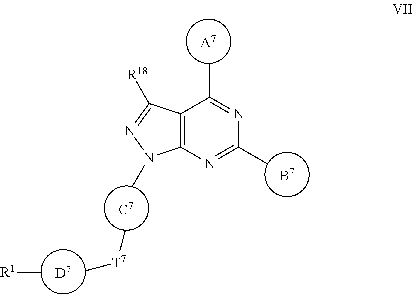
or a pharmaceutically acceptable salt thereof, wherein:
R¹ is a warhead group;
Ring A⁷ is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R¹⁸ is R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B⁷ is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T⁷ is a covalent bond or a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C⁷ is an optionally substituted ring selected from a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; and
Ring D⁷ is absent or an optionally substituted ring selected from a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

260-267. (canceled)

268. A compound of formula VIII: 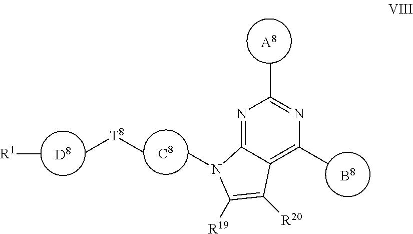
or a pharmaceutically acceptable salt thereof, wherein:
R¹ is a warhead group;
Ring A⁸ is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R¹⁹ and R²⁰ are independently R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B⁸ is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T⁸ is a covalent bond or a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C⁸ is an optionally substituted ring selected from a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; and
Ring D⁸ is absent or an optionally substituted ring selected from a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

269. (canceled)

270. A compound of formula IX: 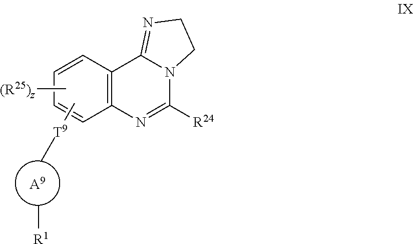
or a pharmaceutically acceptable salt thereof, wherein:
R¹ is a warhead group;
T⁹ is a covalent bond or a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring A⁹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
R²⁴ and R²⁵ are independently R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur; and
z is 0, 1, or 2.

271-273. (canceled)

274. A compound of formula X: 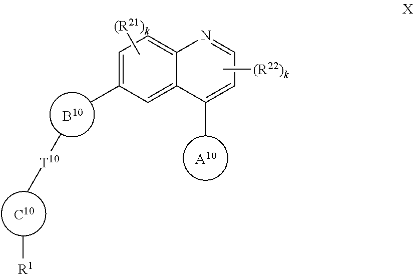
or a pharmaceutically acceptable salt thereof, wherein:
R¹ is a warhead group;
each R²¹ and R²² is independently —R″, halogen, —NO₂, —CN, —OR″, —SR″, —N(R″)₂, —C(O)R″, —CO₂R″, —C(O)C(O)R″, —C(O)CH₂C(O)R″, —S(O)R″, —S(O)₂R″, —C(O)N(R″)₂, —SO₂N(R″)₂, —OC(O)R″, —N(R″)C(O)R″, —N(R″)N(R″)₂, —N(R″)C(═NR″)N(R″)₂, —C(═NR″)N(R″)₂, —C═NOR″, —N(R″)C(O)N(R″)₂, —N(R″)SO₂N(R″)₂, —N(R″)SO₂R″, or —OC(O)N(R″)₂;
each R″ is independently hydrogen or an optionally substituted group selected from C₁₋₆ aliphatic, a 3-7 membered saturated or partially unsaturated carbocyclic ring, a 7-10 membered saturated or partially unsaturated bicyclic carbocyclic ring, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, phenyl, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; or
two R″ groups on the same nitrogen are taken together with the nitrogen to which they are attached to form an optionally substituted 5-8 membered saturated, partially unsaturated, or aromatic ring having 1-4 heteroatoms independently selected from nitrogen, oxygen, or sulfur;
each k is independently 0, 1, or 2;
Ring A¹⁰ is an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
Ring B¹⁰ is an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T¹⁰ is a covalent bond or a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring C¹⁰ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

275-277. (canceled)

278. A compound of formula XI: 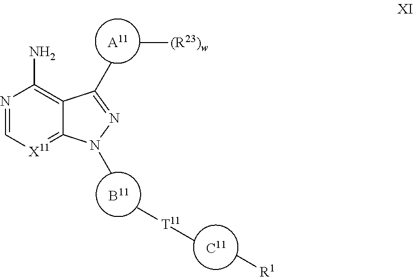
or a pharmaceutically acceptable salt thereof, wherein:
R¹ is a warhead group;
X¹¹ is CH or N;
Ring A¹¹ is an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
each R²³ is independently —R^{a}, halogen, —NO₂, —CN, —OR^{b}, —SR^{b}, —N(R^{b})₂, —C(O)R^{a}, —CO₂R^{a}, —C(O)C(O)R^{a}, —C(O)CH₂C(O)R^{a}, —S(O)R^{a}, —S(O)₂R^{a}, —C(O)N(R^{a})₂, —SO₂N(R^{a})₂, —OC(O)R^{a}, —N(R^{a})C(O)R^{a}, —N(R^{a})N(R^{a})₂, —N(R^{a})C(═NR^{a})N(R^{a})₂, —C(═NR^{a})N(R^{a})₂, —C═NOR^{a}, —N(R^{a})C(O)N(R^{a})₂, —N(R^{a})SO₂N(R^{a})₂, —N(R^{a})SO₂R^{a}, or —OC(O)N(R^{a})₂;
each R^{a} is independently hydrogen, C₁₋₆ aliphatic, phenyl, a 3-7 membered saturated or partially unsaturated carbocyclic ring, a 7-10 membered saturated or partially unsaturated bicyclic carbocyclic ring, a 4-7 membered saturated or partially unsaturated heterocyclic ring having 1-2 heteroatoms independently selected from nitrogen, oxygen, or sulfur, a 7-10 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur; or
two R^{a} groups on the same nitrogen are taken together with the nitrogen to which they are attached to form an optionally substituted 5-8 membered saturated, partially unsaturated, or aromatic ring having 1-4 heteroatoms independently selected from nitrogen, oxygen, or sulfur;
each R^{b} is independently hydrogen, C, aliphatic, a 3-7 membered saturated or partially unsaturated carbocyclic ring, a 7-10 membered saturated or partially unsaturated bicyclic carbocyclic ring, a 4-7 membered saturated or partially unsaturated heterocyclic ring having 1-2 heteroatoms independently selected from nitrogen, oxygen, or sulfur, or a 7-10 membered saturated or partially unsaturated bicyclic heterocyclic ring having 1-3 heteroatoms independently selected from nitrogen, oxygen, or sulfur; or
two R^{b} groups on the same nitrogen are taken together with the nitrogen to which they are attached to form an optionally substituted 5-8 membered saturated, partially unsaturated, or aromatic ring having 1-4 heteroatoms independently selected from nitrogen, oxygen, or sulfur;
w is 0, 1, or 2;
Ring B¹¹ is an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T¹¹ is a covalent bond or a bivalent straight or branched, saturated or unsaturated C₁₋₆ hydrocarbon chain wherein one or more methylene units of T are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring C¹¹ is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

279-285. (canceled)

286. A compound of formula XII: 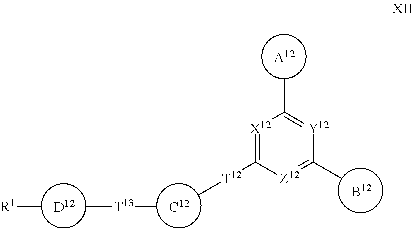
or a pharmaceutically acceptable salt thereof, wherein:
R¹ is a warhead group;
X¹² is CR²⁶ or N;
Y¹² is CR²⁷ or N;
Z¹² is CR²⁸ or N;
wherein at least one of X¹², Y¹², and Z¹² is N;
Ring A¹² is an optionally substituted ring selected from a 4-8 membered saturated or partially unsaturated heterocyclic ring having one or two heteroatoms independently selected from nitrogen, oxygen, or sulfur, or a 5-15 membered saturated or partially unsaturated bridged or spiro bicyclic heterocyclic ring having at least one nitrogen, at least one oxygen, and optionally 1-2 additional heteroatoms independently selected from nitrogen, oxygen, or sulfur;
R²⁶, R²⁷, and R²⁸ are independently R, halogen, —OR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen or an optionally substituted group selected from C₁₋₆ aliphatic, phenyl, a 4-7 membered heterocylic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur, or:
two R groups on the same nitrogen are taken together with the nitrogen atom to which they are attached to form a 4-7 membered saturated, partially unsaturated, or heteroaryl ring having 1-4 heteroatoms independently selected from nitrogen, oxygen, or sulfur;
Ring B¹² is an optionally substituted group selected from phenyl, an 8-10 membered bicyclic aryl ring, a 5-6 membered heteroaryl ring having 1-4 heteroatoms independently selected from nitrogen, oxygen, or sulfur, or an 8-10 membered bicyclic heteroaryl ring having 1-4 heteroatoms independently selected from nitrogen, oxygen, or sulfur;
T¹² is a covalent bond or a bivalent straight or branched, saturated or unsaturated C₁₋₆ hydrocarbon chain wherein one or more methylene units of T¹² are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—;
Ring C¹² is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged or spiro bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur;
T¹³ is a covalent bond or a bivalent straight or branched, saturated or unsaturated C₁₋₆ hydrocarbon chain wherein one or more methylene units of T¹³ are optionally replaced by —O—, —S—, —N(R)—, —C(O)—, —OC(O)—, —C(O)O—, —C(O)N(R)—, —N(R)C(O)—, —N(R)C(O)N(R)—, —SO₂—, —SO₂N(R)—, —N(R)SO₂—, or —N(R)SO₂N(R)—; and
Ring D¹² is absent or an optionally substituted ring selected from phenyl, a 3-7 membered saturated or partially unsaturated carbocyclic ring, a 7-10 membered saturated or partially unsaturated bicyclic carbocyclic ring, a 7-12 membered saturated or partially unsaturated bridged bicyclic ring having 0-4 heteroatoms independently selected from nitrogen, oxygen, or sulfur, a 4-7 membered saturated or partially unsaturated heterocyclic ring having 1-2 heteroatoms independently selected from nitrogen, oxygen, or sulfur, a 7-12 membered saturated or partially unsaturated bicyclic heterocyclic ring having 1-3 heteroatoms independently selected from nitrogen, oxygen, or sulfur, an 8-10 membered bicyclic aryl ring, a 5-6 membered heteroaryl ring having 1-3 heteroatoms independently selected from nitrogen, oxygen, or sulfur, or an 8-10 membered bicyclic heteroaryl ring having 1-4 heteroatoms independently selected from nitrogen, oxygen, or sulfur.

287. The compound according to claim 286, wherein the compound is of formula XII-a:

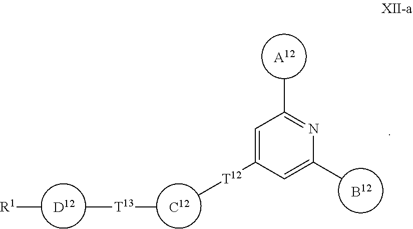

288-343. (canceled)

344. The compound according to claim 55, wherein R¹ is -L-Y, wherein:
L is a bivalent C₂₋₈ straight or branched, hydrocarbon chain wherein L has at least one double bond and one or two additional methylene units of L are optionally and independently replaced by —NRC(O)—, —C(O)NR—, —N(R)SO₂—, —SO₂N(R)—, —S—, —S(O)—, —SO₂—, —OC(O)—, —C(O)O—, cyclopropylene, —O—, —N(R)—, or —C(O)—;
Y is hydrogen, C₁₋₆ aliphatic optionally substituted with oxo, halogen, NO₂, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with 1-4 R^{e} groups; and
each R^{e} is independently selected from -Q-Z, oxo, NO₂, halogen, CN, a suitable leaving group, or C₁₋₆ aliphatic optionally substituted with oxo, halogen, NO₂, or CN, wherein:
Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, or —SO₂—, —N(R)C(O)—, —C(O)N(R)—, —N(R)SO₂—, or —SO₂N(R)—; and
Z is hydrogen or C₁₋₆ aliphatic optionally substituted with oxo, halogen, NO₂, or CN.

345. The compound according to claim 344, wherein:
L is a bivalent C₂₋₈ straight or branched, hydrocarbon chain wherein L has at least one double bond and at least one methylene unit of L is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L are optionally and independently replaced by cyclopropylene, —O—, —N(R)—, or —C(O)—; and
Y is hydrogen or C₁₋₆ aliphatic optionally substituted with oxo, halogen, NO₂, or CN.

346. The compound according to claim 345, wherein L is a bivalent C₂₋₈ straight or branched, hydrocarbon chain wherein L has at least one double bond and at least one methylene unit of L is replaced by —C(O)—, and one or two additional methylene units of L are optionally and independently replaced by cyclopropylene, —O—, —N(R)—, or —C(O)—.

347. The compound according to claim 345, wherein L is a bivalent C₂₋₈ straight or branched, hydrocarbon chain wherein L has at least one double bond and at least one methylene unit of L is replaced by —OC(O)—.

348. The compound according to claim 344, wherein L is —NRC(O)CH═CH—, —NRC(O)CH═CHCH₂N(CH₃)—, —NRC(O)CH═CHCH₂O—, —CH₂NRC(O)CH═CH—, —NRSO₂CH═CH—, —NRSO₂CH═CHCH₂—, —NRC(O)(C═N₂)—, —NRC(O)(C═N₂)C(O)—, —NRC(O)CH═CHCH₂N(CH₃)—, —NRSO₂CH═CH—, —NRSO₂CH═CHCH₂—, —NRC(O)CH═CHCH₂O—, —NRC(O)C(═CH₂)CH₂—, —CH₂NRC(O)—, —CH₂NRC(O)CH═CH—, —CH₂CH₂NRC(O)—, or —CH₂NRC(O)cyclopropylene-; wherein R is H or optionally substituted C₁₋₆ aliphatic; and Y is hydrogen or C₁₋₆ aliphatic optionally substituted with oxo, halogen, NO₂, or CN.

349. The compound according to claim 348, wherein L is —NHC(O)CH═CH—, —NHC(O)CH═CHCH₂N(CH₃)—, —NHC(O)CH═CHCH₂O—, —CH₂NHC(O)CH═CH—, —NHSO₂CH═CH—, —NHSO₂CH═CHCH₂—, —NHC(O)(C═N₂)—, —NHC(O)(C═N₂)C(O)—, —NHC(O)CH═CHCH₂N(CH₃)—, —NHSO₂CH═CH—, —NHSO₂CH═CHCH₂—, —NHC(O)CH═CHCH₂O—, —NHC(O)C(═CH₂)CH₂—, —CH₂NHC(O)—, —CH₂NHC(O)CH═CH—, —CH₂CH₂NHC(O)—, or —CH₂NHC(O)cyclopropylene-.

350. The compound according to claim 344, wherein L is a bivalent C₂₋₈ straight or branched, hydrocarbon chain wherein L has at least one alkylidenyl double bond and at least one methylene unit of L is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L are optionally and independently replaced by cyclopropylene, —O—, —N(R)—, or —C(O)—.

351. The compound according to claim 55, wherein R¹ is -L-Y, wherein:
L is a bivalent C₂₋₈ straight or branched, hydrocarbon chain wherein L has at least one triple bond and one or two additional methylene units of L are optionally and independently replaced by —NRC(O)—, —C(O)NR—, —N(R)SO₂—, —SO₂N(R)—, —S—, —S(O)—, —SO₂—, —OC(O)—, or —C(O)O—,
Y is hydrogen, C₁₋₆ aliphatic optionally substituted with oxo, halogen, NO₂, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with 1-4 R^{e} groups; and
each R^{e} is independently selected from -Q-Z, oxo, NO₂, halogen, CN, a suitable leaving group, or C₁₋₆ aliphatic optionally substituted with oxo, halogen, NO₂, or CN, wherein:
Q is a covalent bond or a bivalent C₁₋₆ saturated or unsaturated, straight or branched, hydrocarbon chain, wherein one or two methylene units of Q are optionally and independently replaced by —N(R)—S—, —O—, —C(O)—, —OC(O)—, —C(O)O—, —SO—, or —SO₂—, —N(R)C(O)—, —C(O)N(R)—, —N(R)SO₂—, or —SO₂N(R)—; and
Z is hydrogen or C₁₋₆ aliphatic optionally substituted with oxo, halogen, NO₂, or CN.

352. The compound according to claim 351, wherein Y is hydrogen or C₁₋₆ aliphatic optionally substituted with oxo, halogen, NO₂, or CN.

353. The compound according to claim 352, wherein L is —C≡C—, —C≡CCH₂N(isopropyl)-, —NHC(O)C≡CCH₂CH₂—, —CH₂—C≡C—CH₂—, —C≡CCH₂O—, —CH₂C(O)C≡C—, —C(O)C≡C—, or —CH₂C(═O)C≡C—.

354. The compound according to claim 55, wherein R¹ is -L-Y, wherein:
L is a bivalent C₂₋₈ straight or branched, hydrocarbon chain wherein one methylene unit of L is replaced by cyclopropylene and one or two additional methylene units of L are independently replaced by —NRC(O)—, —C(O)NR—, —N(R)SO₂—, —SO₂N(R)—, —S—, —S(O)—, —SO₂—, —OC(O)—, or —C(O)O—;
Y is hydrogen, C₁₋₆ aliphatic optionally substituted with oxo, halogen, NO₂, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with 1-4 R^{e} groups; and
each R^{e} is independently selected from -Q-Z, oxo, NO₂, halogen, CN, a suitable leaving group, or C₁₋₆ aliphatic optionally substituted with oxo, halogen, NO₂, or CN, wherein:
Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, or —SO₂—, —N(R)C(O)—, —C(O)N(R)—, —N(R)SO₂—, or —SO₂N(R)—; and
Z is hydrogen or C₁₋₆ aliphatic optionally substituted with oxo, halogen, NO₂, or CN.

355. The compound according to claim 354, wherein Y is hydrogen or C₁₋₆ aliphatic optionally substituted with oxo, halogen, NO₂, or CN.

356. The compound according to claim 55, wherein R¹ is -L-Y, wherein:
L is a covalent bond, —C(O)—, —N(R)C(O)—, or a bivalent C₁₋₈ saturated or unsaturated, straight or branched, hydrocarbon chain; and
Y is selected from the following (i) through (xvii):
(i) C₁₋₆ alkyl substituted with oxo, halogen, NO₂, or CN;
(ii) C₂₋₆ alkenyl optionally substituted with oxo, halogen, NO₂, or CN; or
(iii) C₂₋₆ alkynyl optionally substituted with oxo, halogen, NO₂, or CN; or
(iv) a saturated 3-4 membered heterocyclic ring having 1 heteroatom selected from oxygen or nitrogen wherein said ring is substituted with 1-2 R^{e} groups; or
(v) a saturated 5-6 membered heterocyclic ring having 1-2 heteroatom selected from oxygen or nitrogen wherein said ring is substituted with 1-4 R^{e} groups; or
(vi) 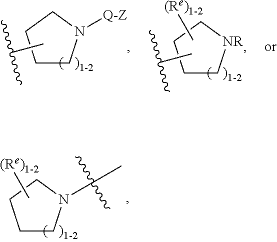
wherein each R, Q, Z; or
(vii) a saturated 3-6 membered carbocyclic ring, wherein said ring is substituted with 1-4 R^{e} groups; or
(viii) a partially unsaturated 3-6 membered monocyclic ring having 0-3 heteroatoms independently selected from nitrogen, oxygen, or sulfur, wherein said ring is substituted with 1-4 R^{e} groups; or
(ix) a partially unsaturated 3-6 membered carbocyclic ring, wherein said ring is substituted with 1-4 R^{e} groups;
(x) 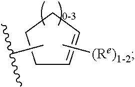
or
(xi) a partially unsaturated 4-6 membered heterocyclic ring having 1-2 heteroatoms independently selected from nitrogen, oxygen, or sulfur, wherein said ring is substituted with 1-4 R^{e} groups; or
(xii) 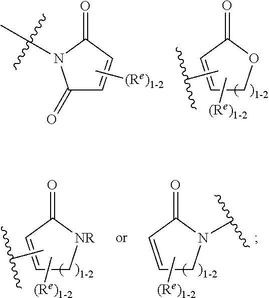
or
(xiii) a 6-membered aromatic ring having 0-2 nitrogens wherein said ring is substituted with 1-4 R^{e} groups; or
(xiv) 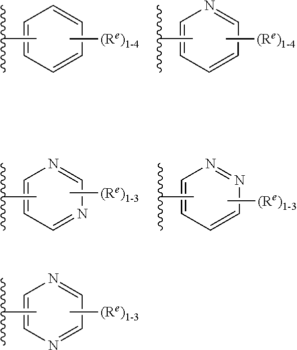
wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups; or
(xvi) 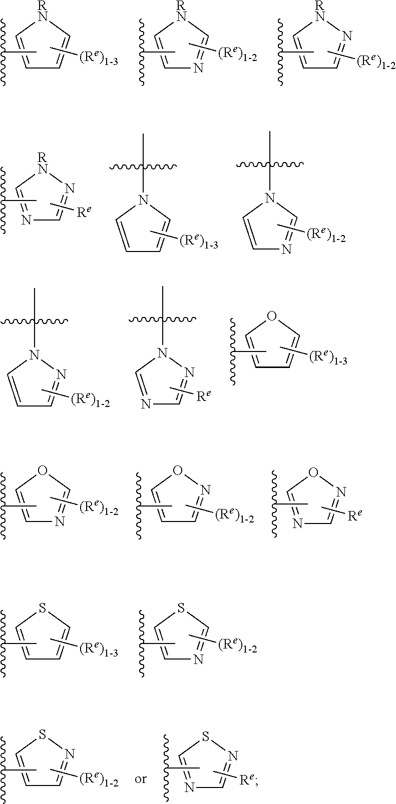
or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups.

357. The compound according to claim 356, wherein L is a covalent bond, —CH₂—, —NH—, —C(O)—, —CH₂NH—, —NHCH₂—, —NHC(O)—, —NHC(O)CH₂OC(O)—, —CH₂NHC(O)—, —NHSO₂—, —NHSO₂CH₂—, —NHC(O)CH₂OC(O)—, or —SO₂NH—.

358. The compound according to claim 357, wherein L is a covalent bond.

359. The compound according claim 356, wherein Y is selected from: 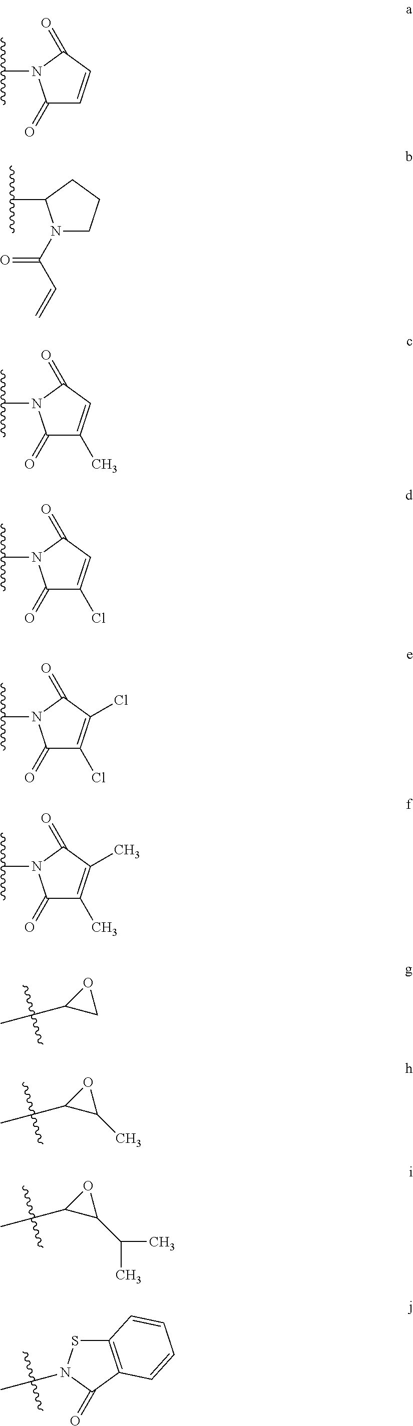 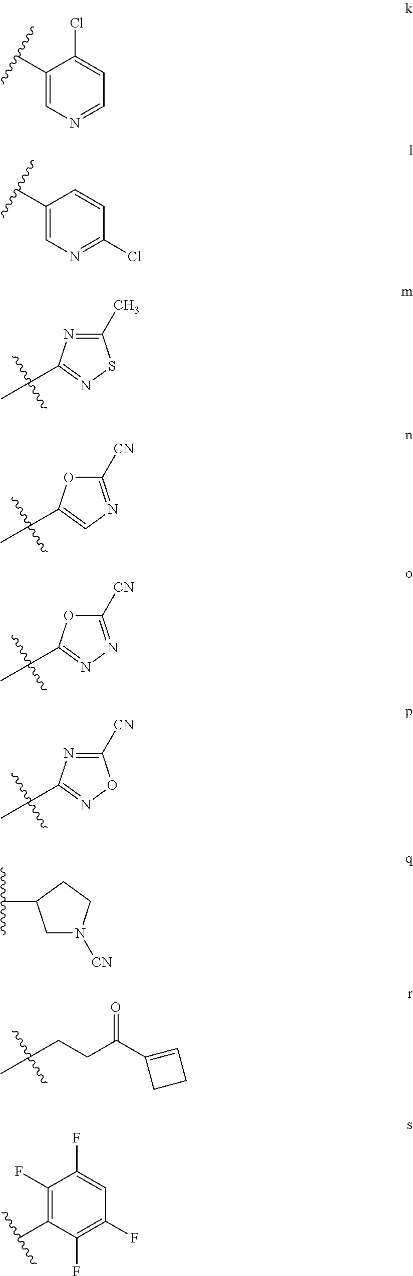 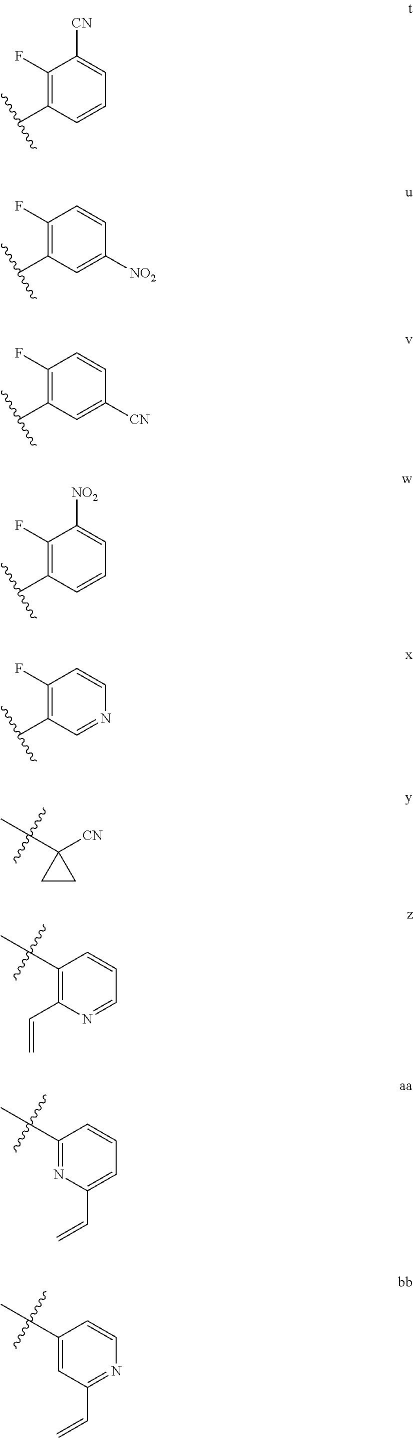 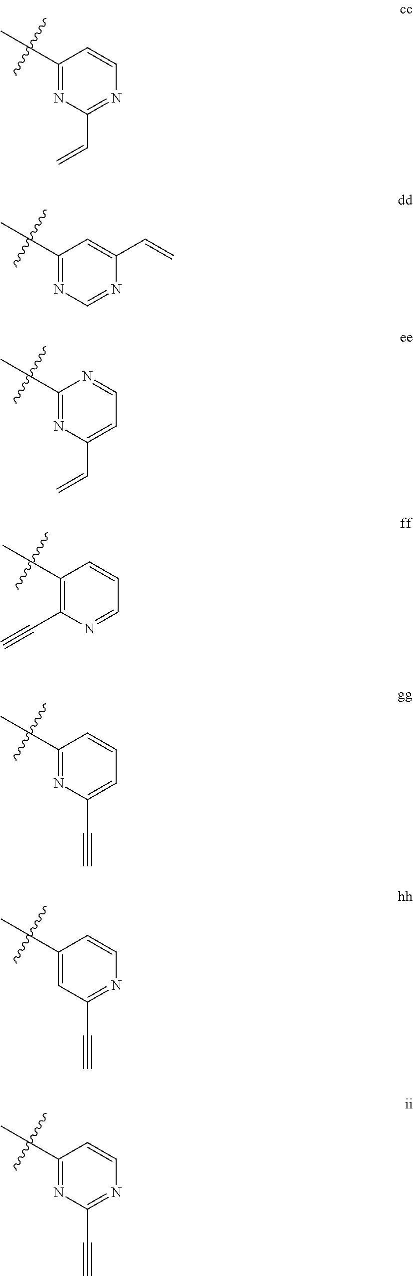 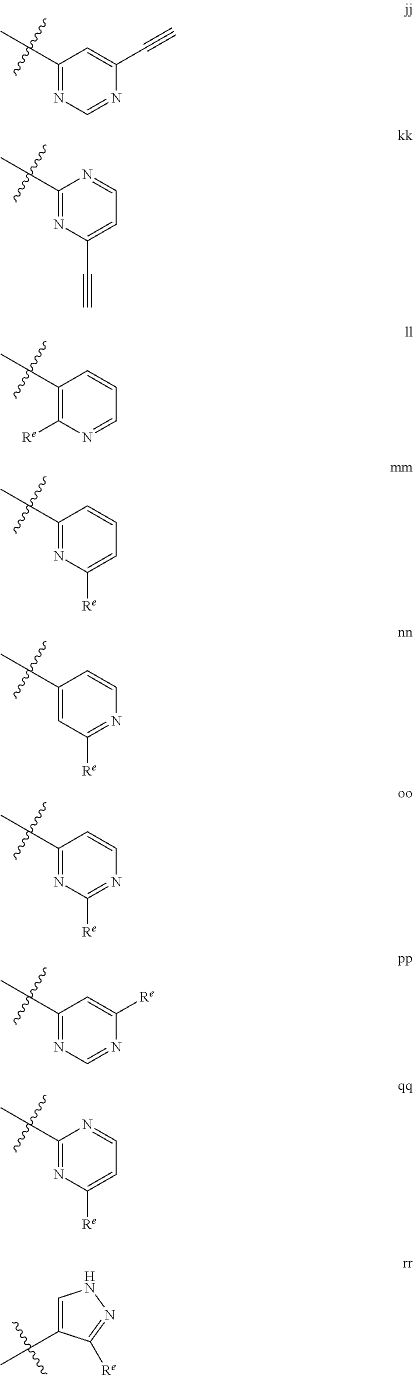 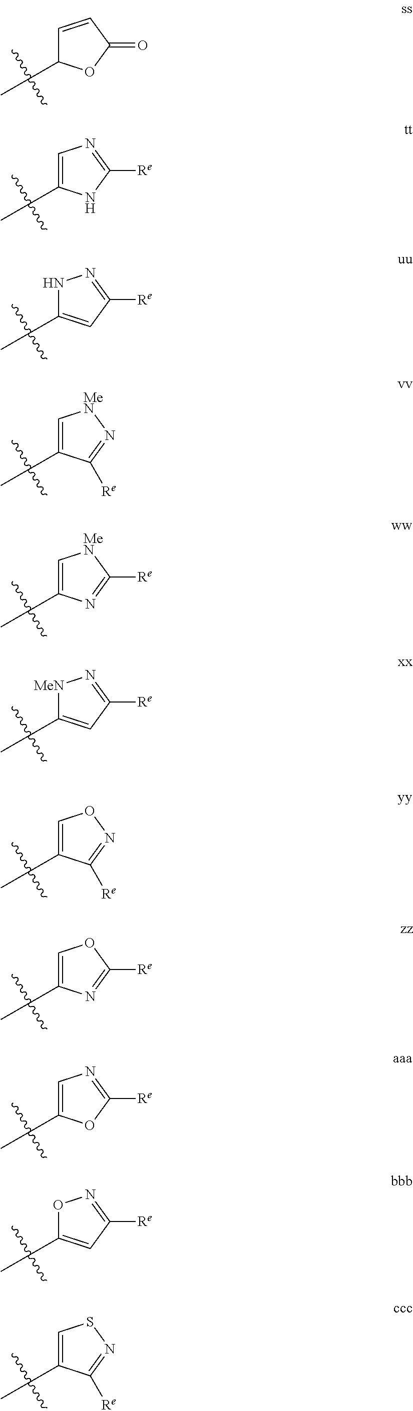 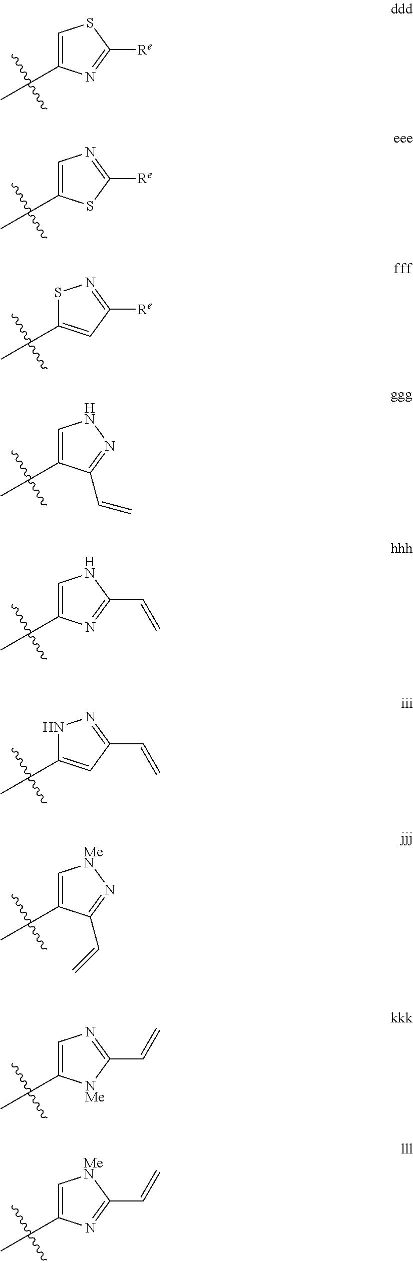 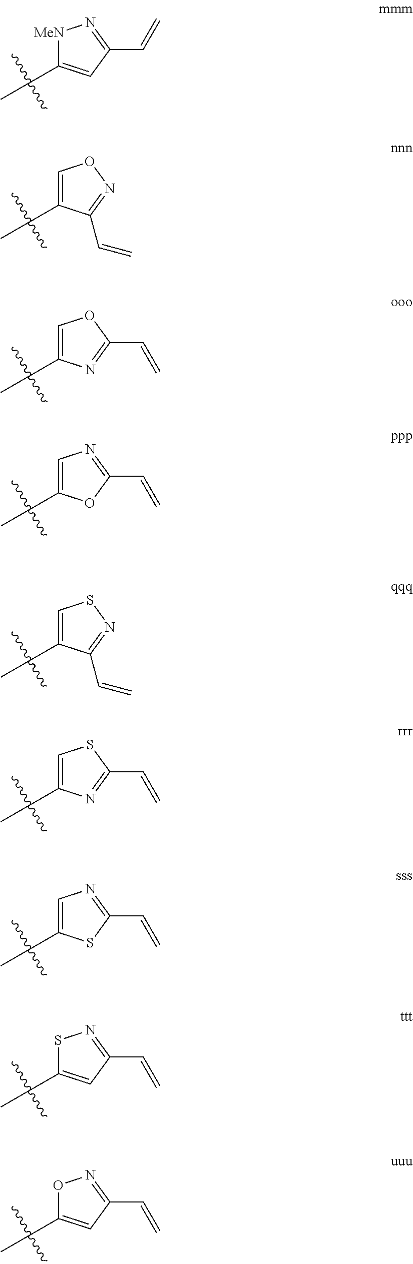 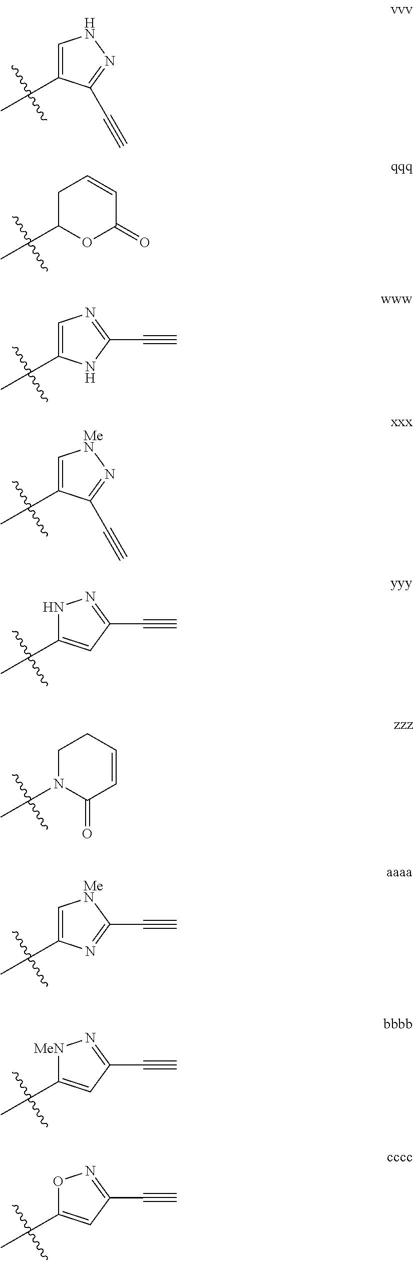 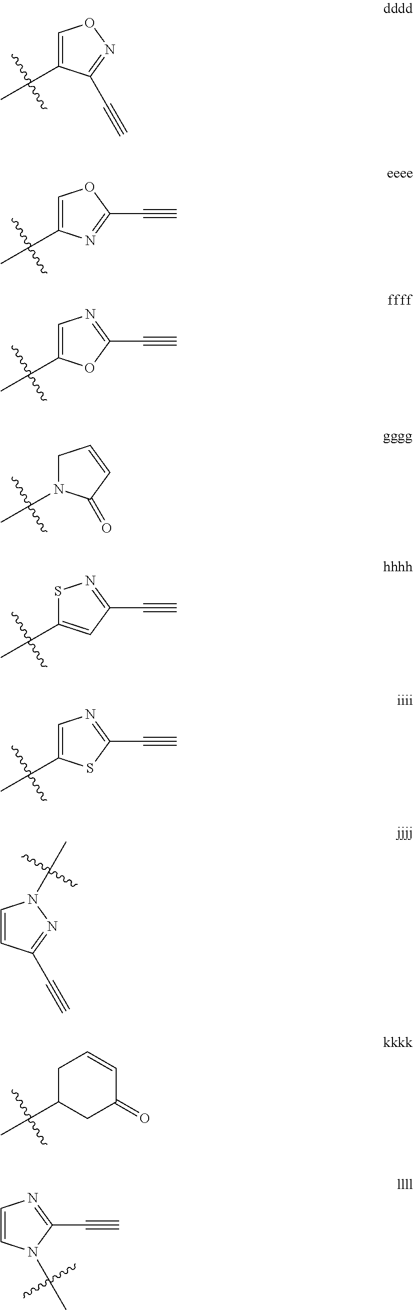 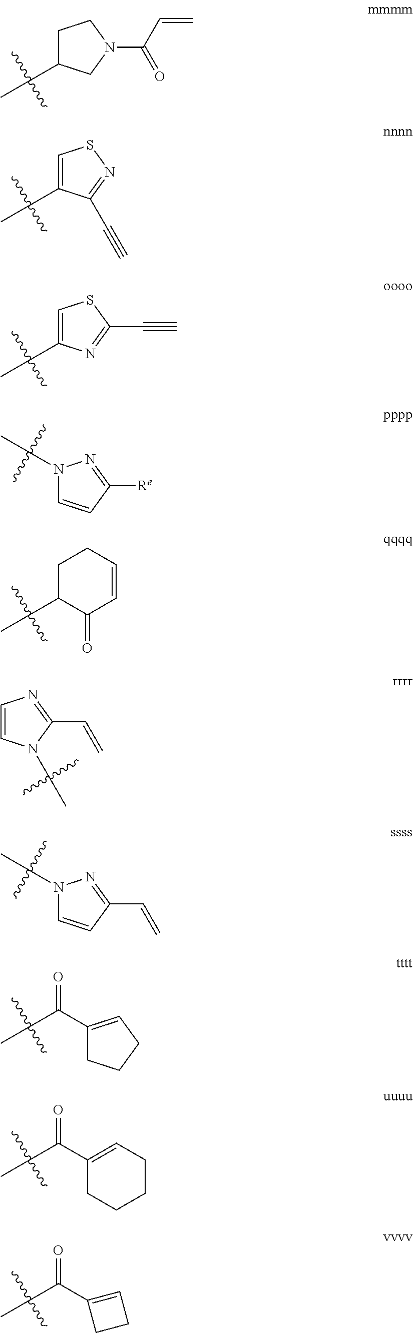 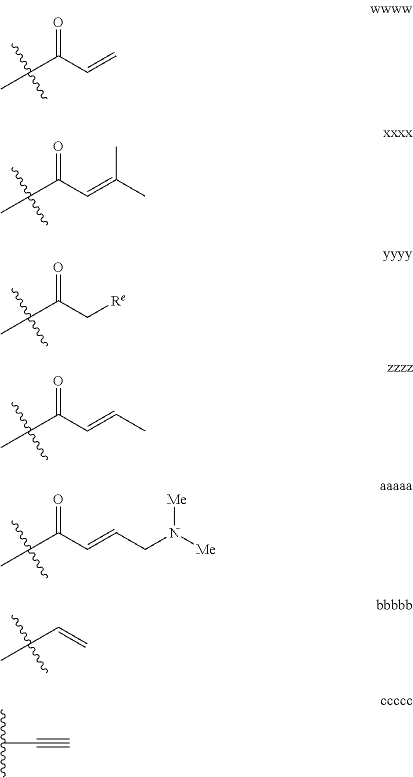
wherein each R^{e} is independently selected from a suitable leaving group, CN, NO₂ or oxo.

360. The compound of claim 55, wherein R¹ is -L-Y, wherein:
L is a bivalent C₂₋₈ straight or branched, hydrocarbon chain wherein two or three methylene units of L are optionally and independently replaced by —NRC(O)—, —C(O)NR—, —N(R)SO₂—, —SO₂N(R)—, —S—, —S(O)—, —SO₂—, —OC(O)—, —C(O)O—, cyclopropylene, —O—, —N(R)—, or —C(O)—; and
Y is hydrogen or C₁₋₆ aliphatic optionally substituted with oxo, halogen, NO₂, or CN.

361. The compound of claim 360, wherein R¹ is —C(O)CH₂CH₂C(O)CH═C(CH₃)₂, —C(O)CH₂CH₂C(O)CH═CH(cyclopropyl), —C(O)CH₂CH₂C(O)CH═CHCH₃, —C(O)CH₂CH₂C(O)CH═CHCH₂CH₃, —C(O)CH₂CH₂C(O)C(═CH₂)CH₃, —C(O)CH₂NHC(O)CH═CH₂, —C(O)CH₂NHC(O)CH₂CH₂C(O)CH═CHCH₃, —C(O)CH₂NHC(O)CH₂CH₂C(O)C(═CH₂)CH₃, —S(O)₂CH₂CH₂NHC(O)CH₂CH₂C(O)CH═C(CH₃)₂, —S(O)₂CH₂CH₂NHC(O)CH₂CH₂C(O)CH═CHCH₃, —S(O)₂CH₂CH₂NHC(O)CH₂CH₂C(O)CH═CH₂, —C(O)(CH₂)₃NHC(O)CH₂CH₂C(O)CH═CHCH₃, or —C(O)(CH₂)₃NHC(O)CH₂CH₂C(O)CH═CH₂.

362. The compound of claim 55, wherein R¹ is 6-12 atoms long.

363. The compound of claim 361, wherein R¹ is at least 8 atoms long.

364. The compound according to claim 55, wherein R¹ is selected from: 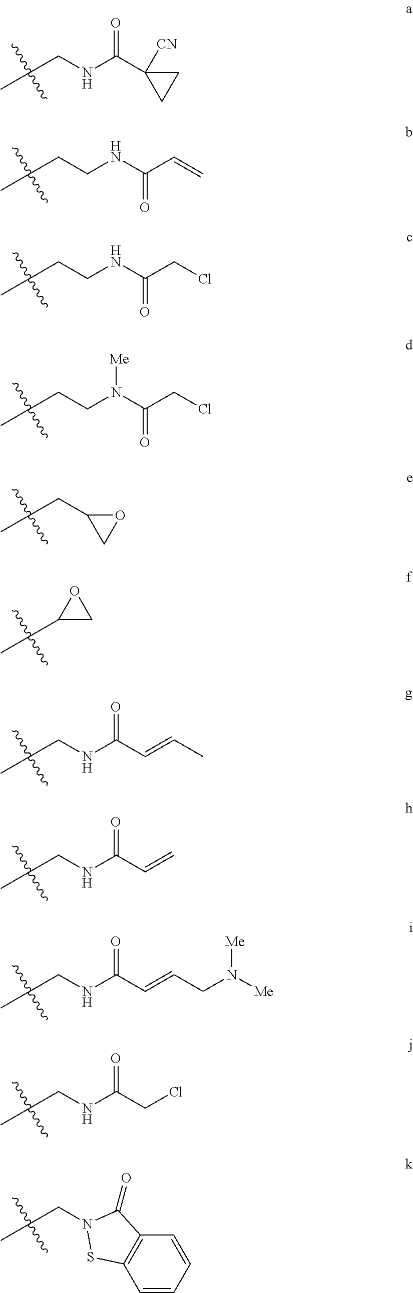 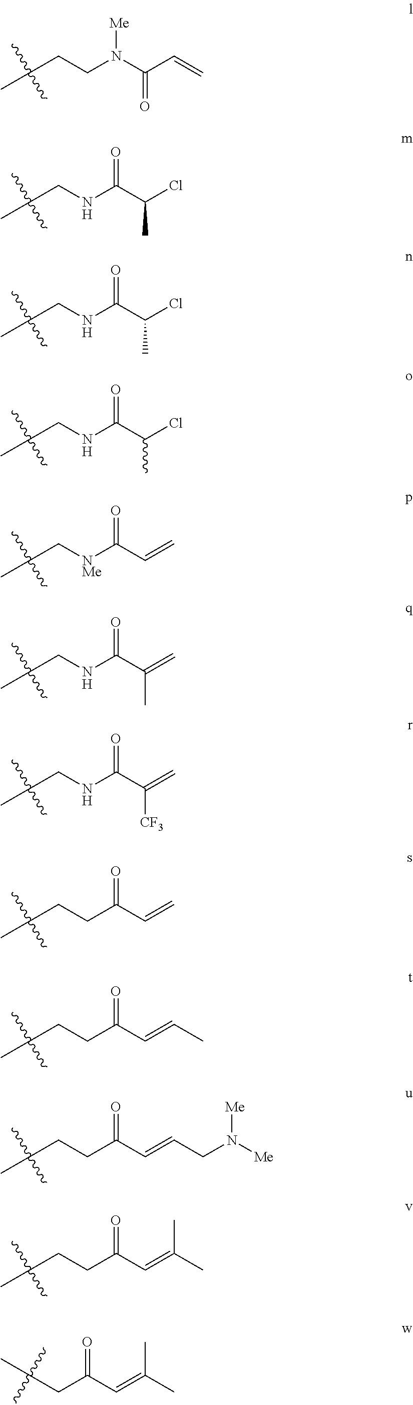 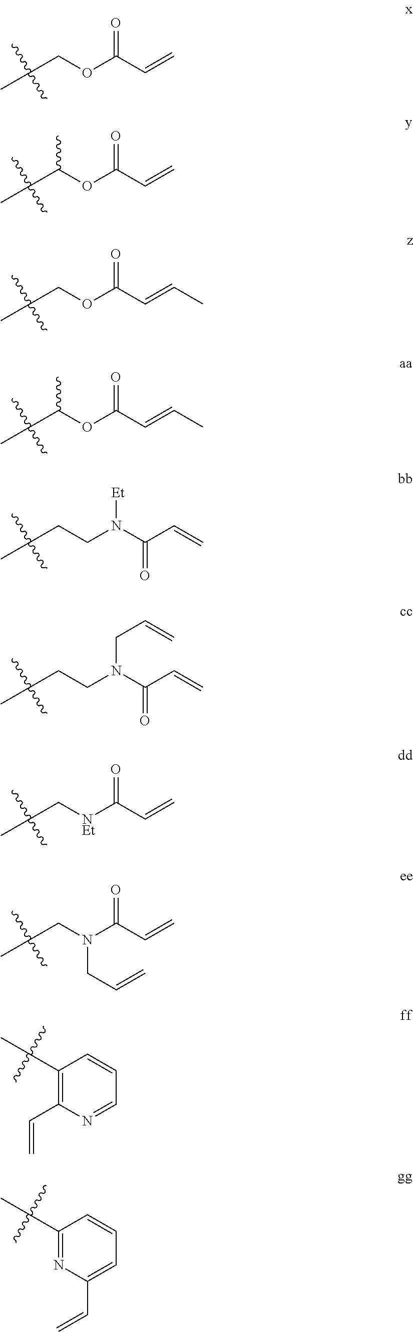 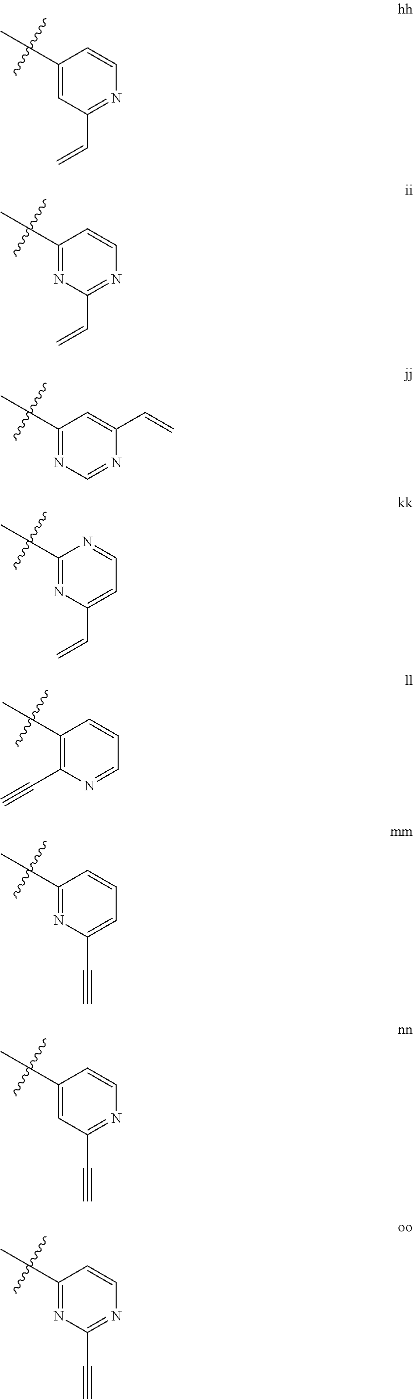 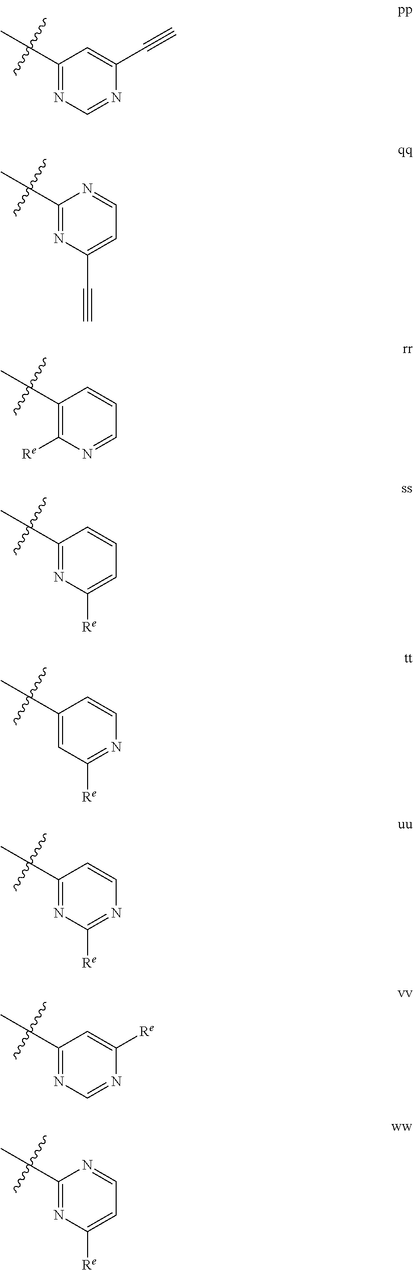 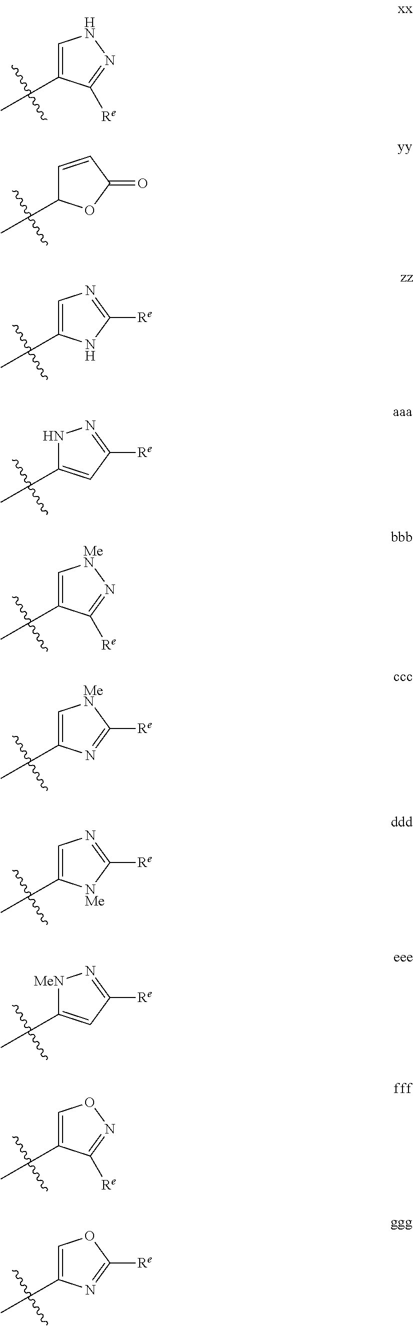 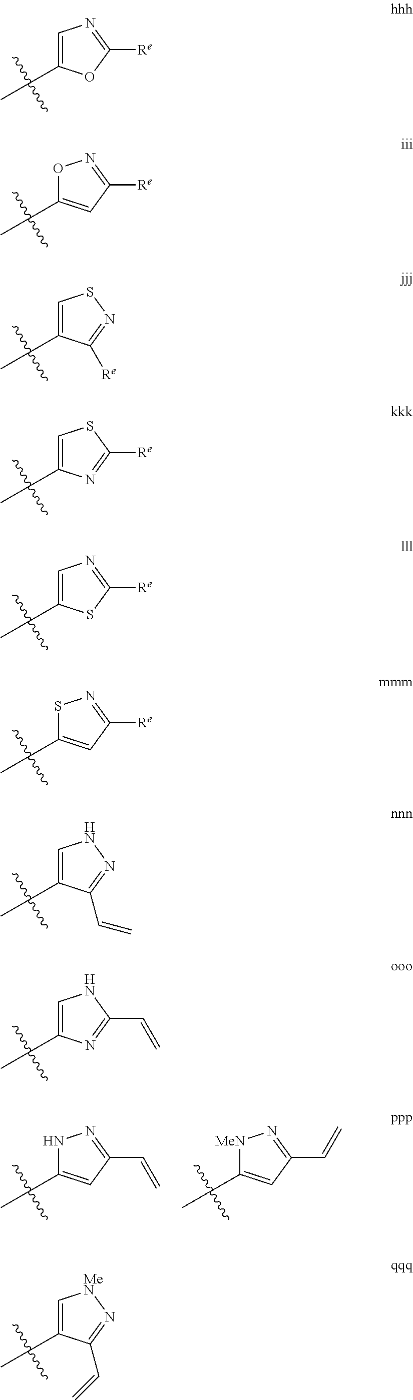 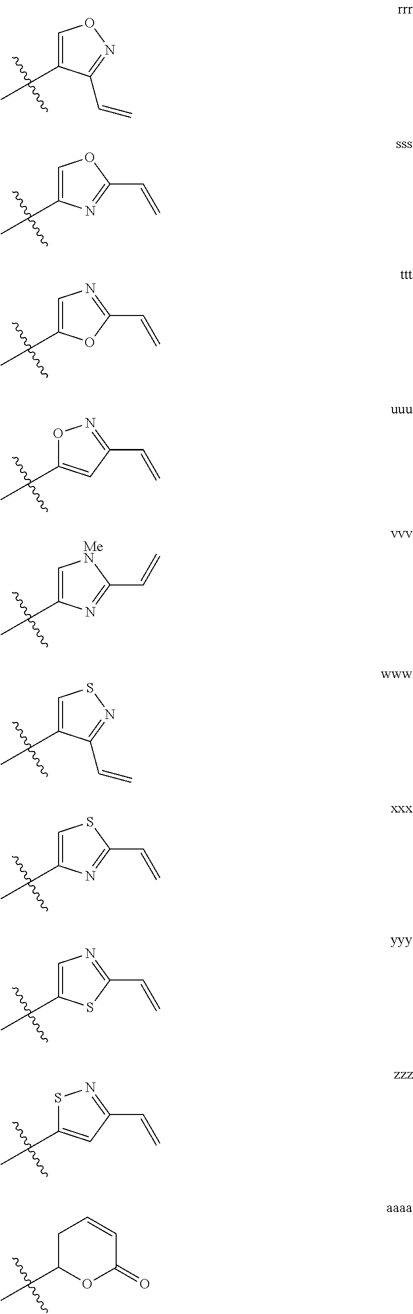 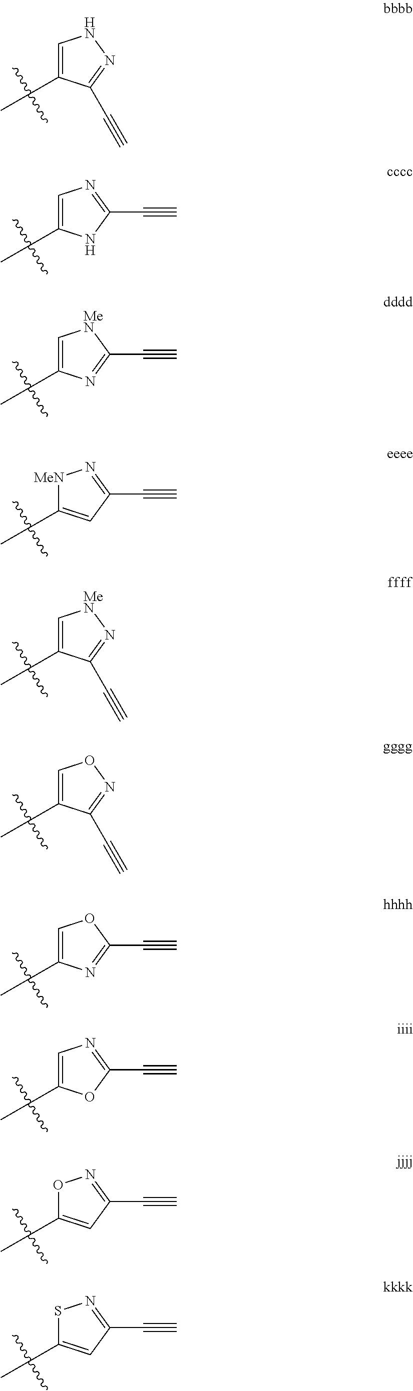 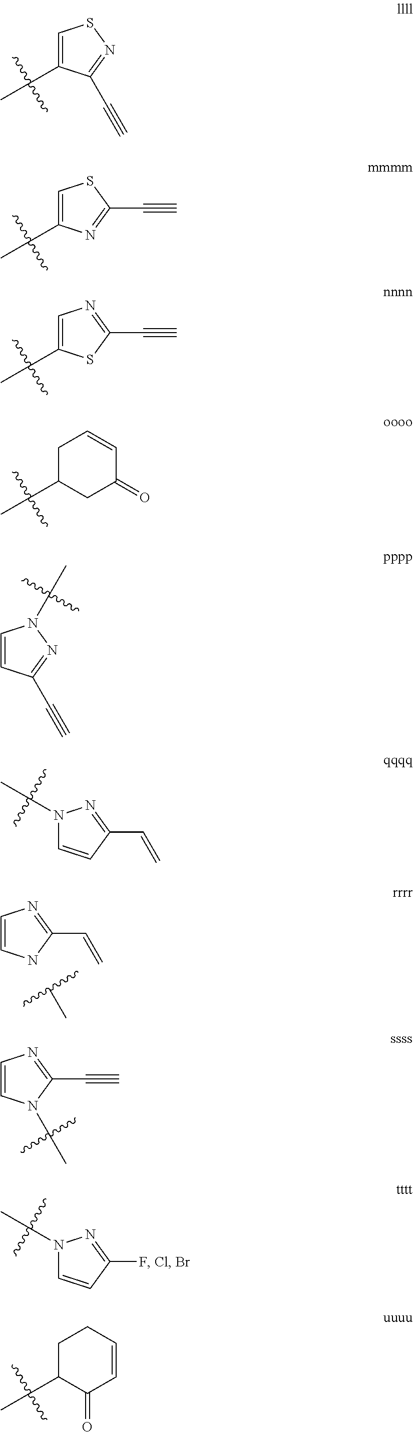 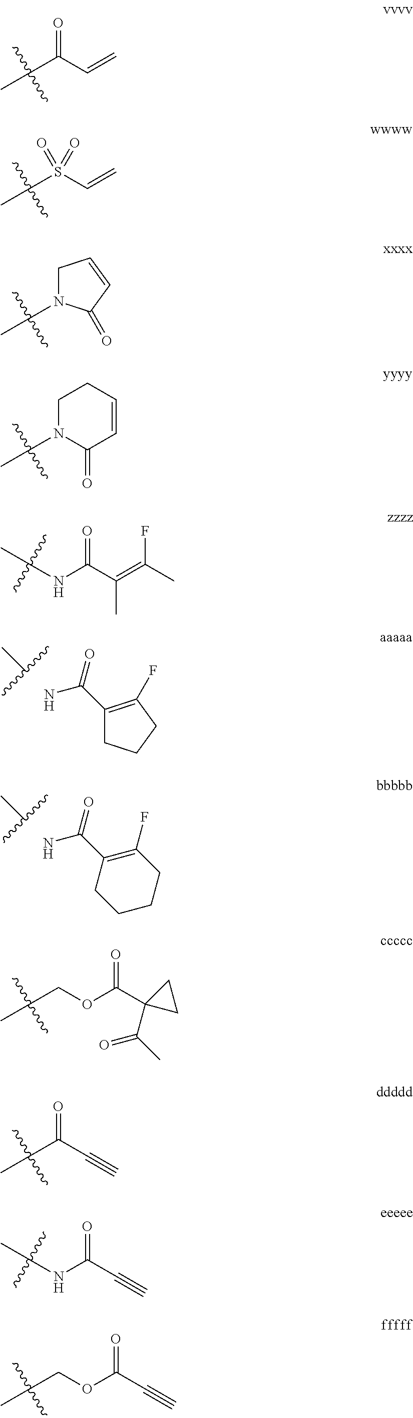 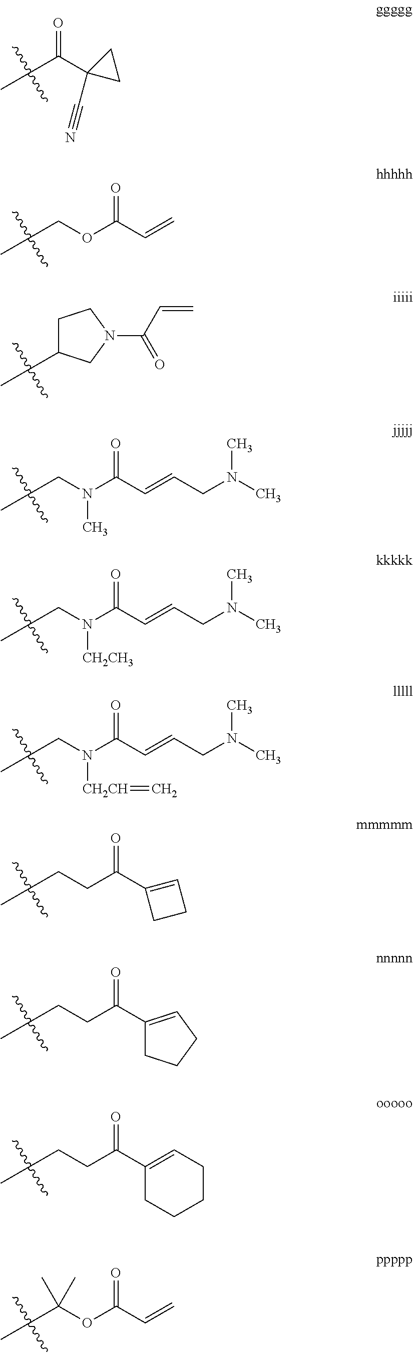 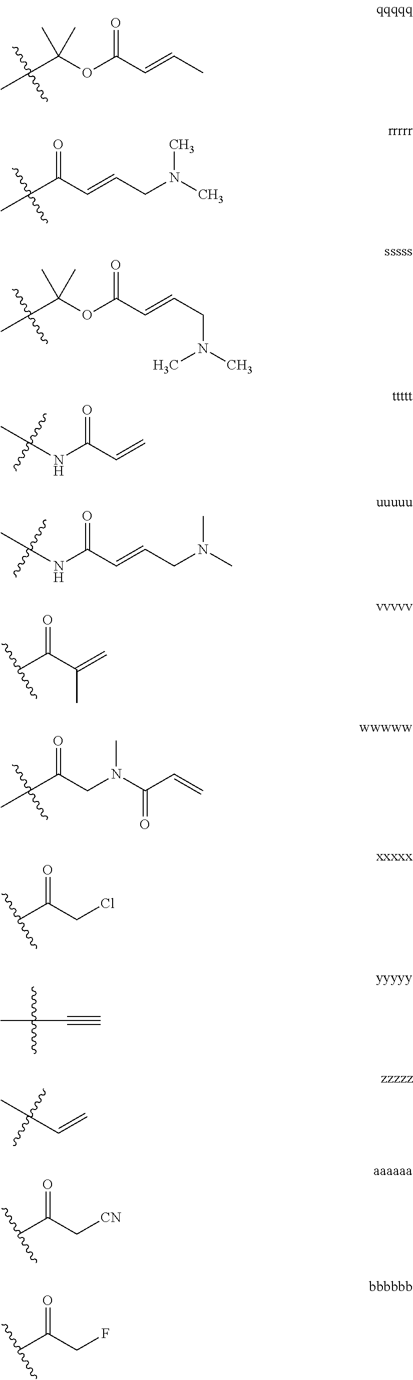 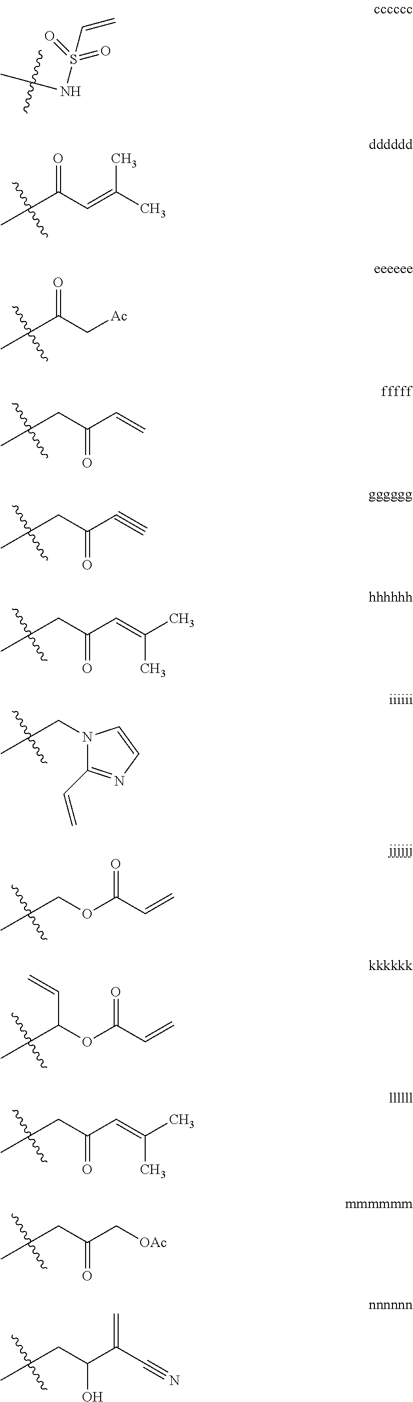 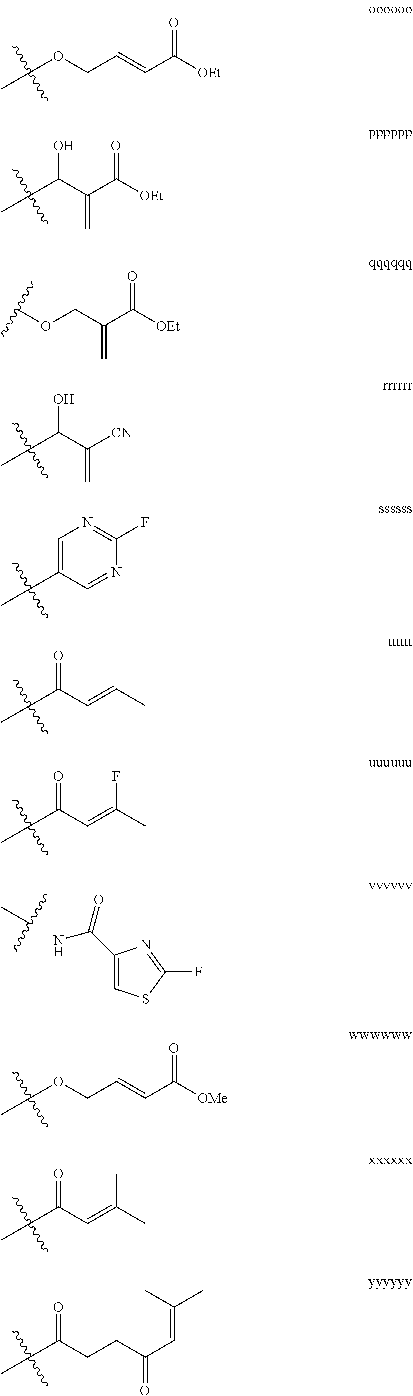 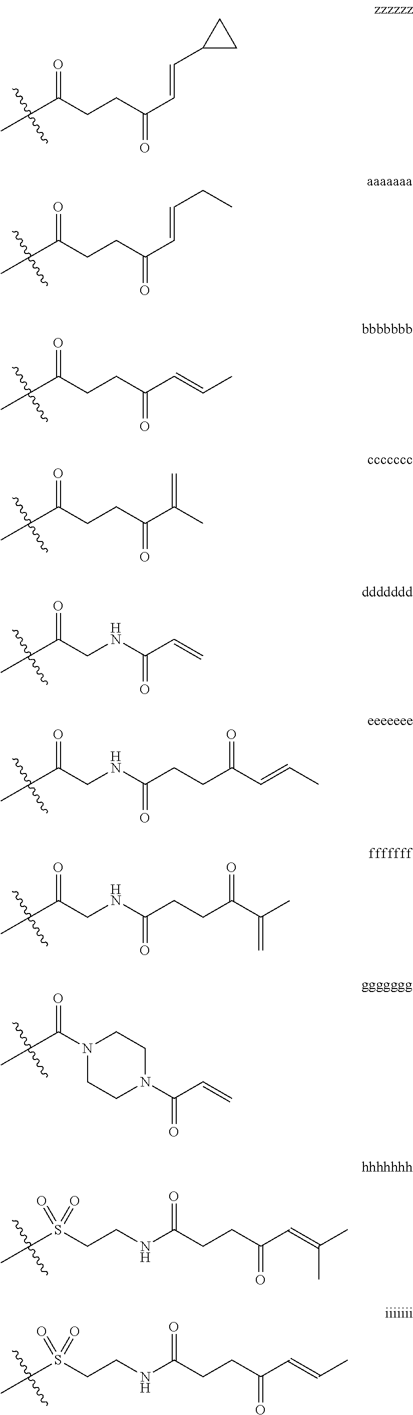 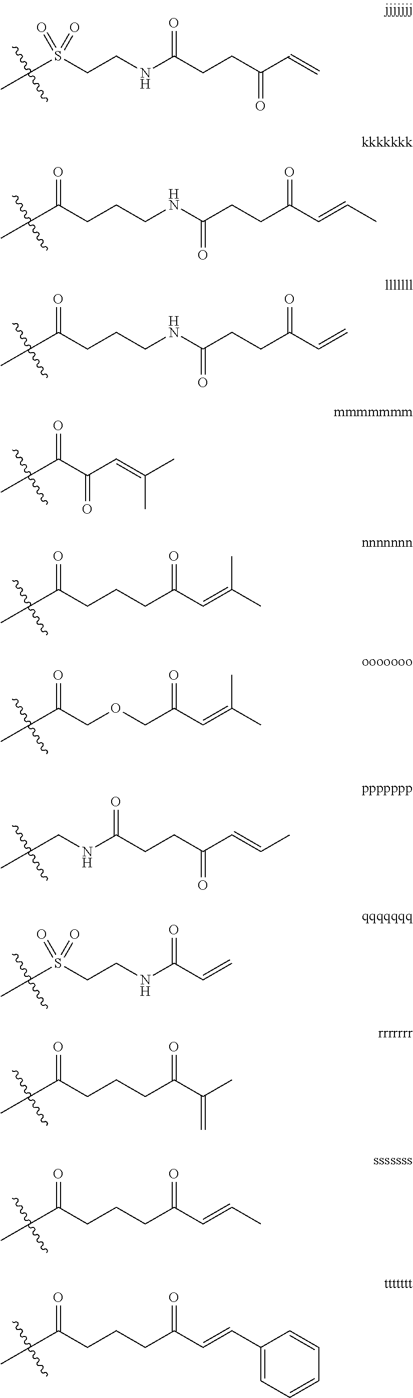 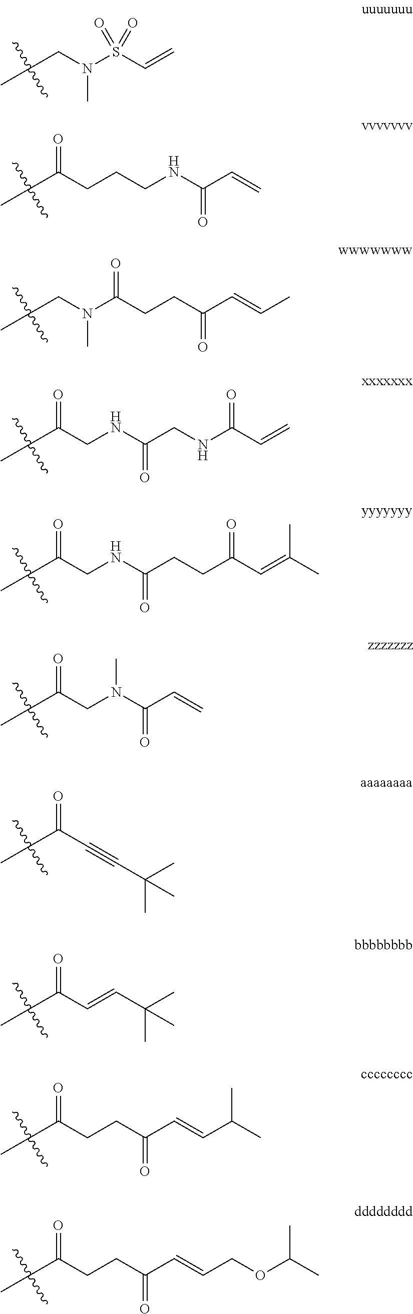 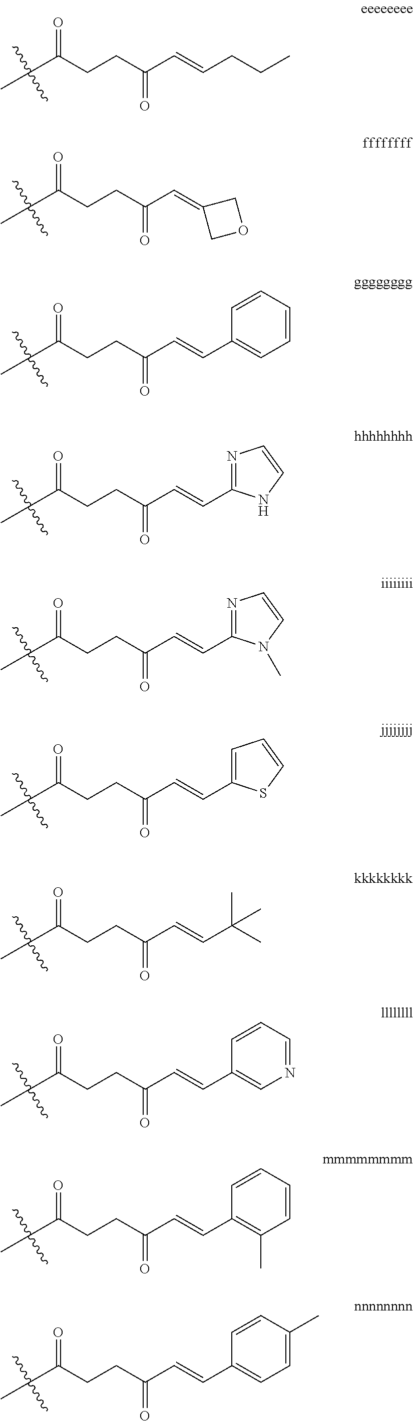 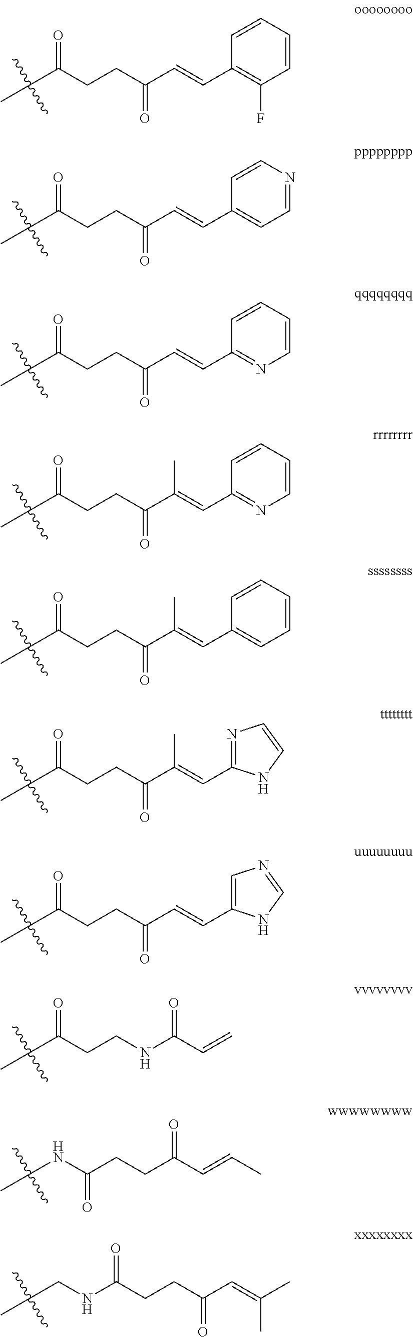 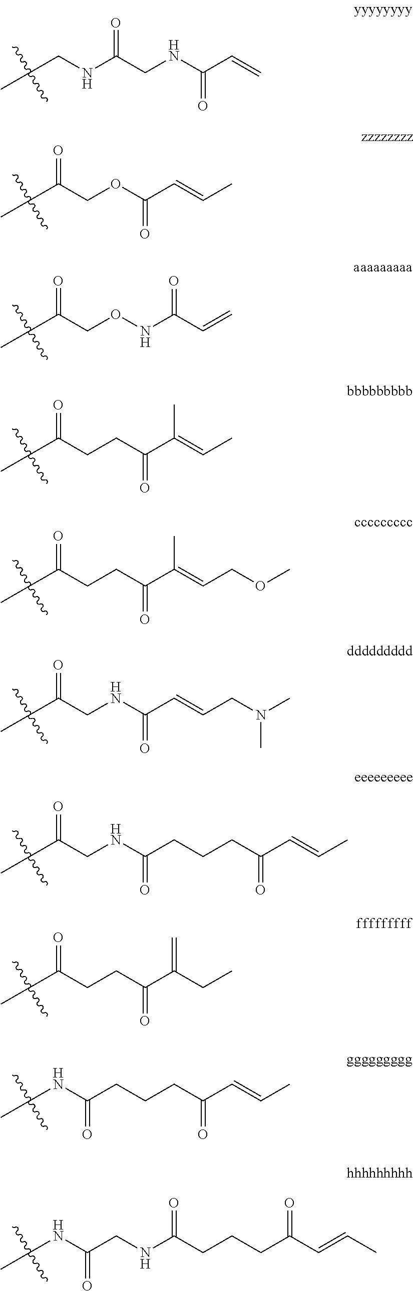 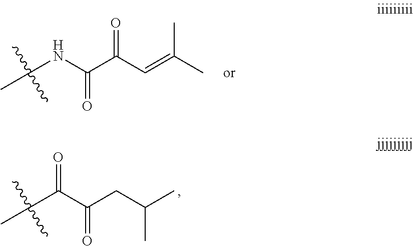
wherein each R^{e} is independently a suitable leaving group, NO₂, CN, or oxo.

365. The compound according to claim 55, wherein R¹ is selected from:

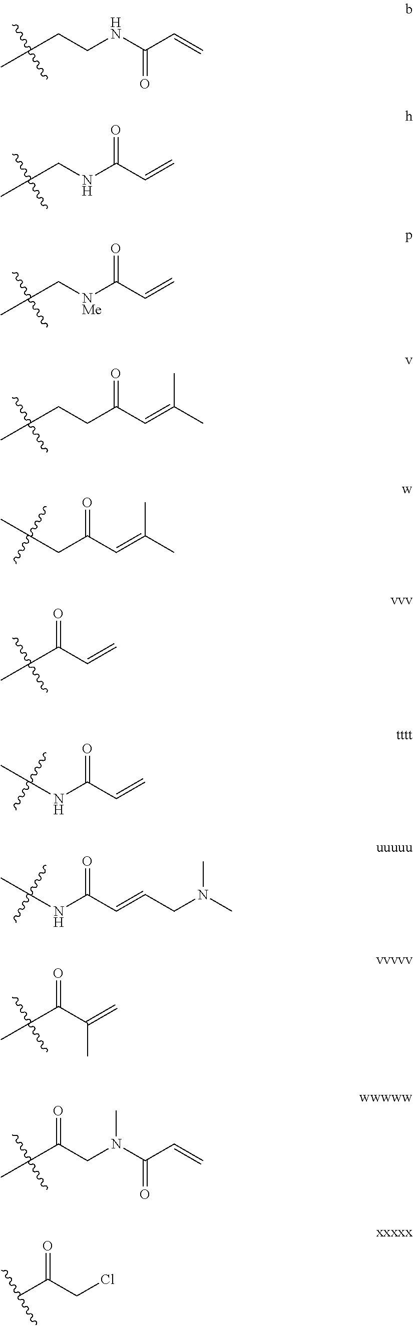

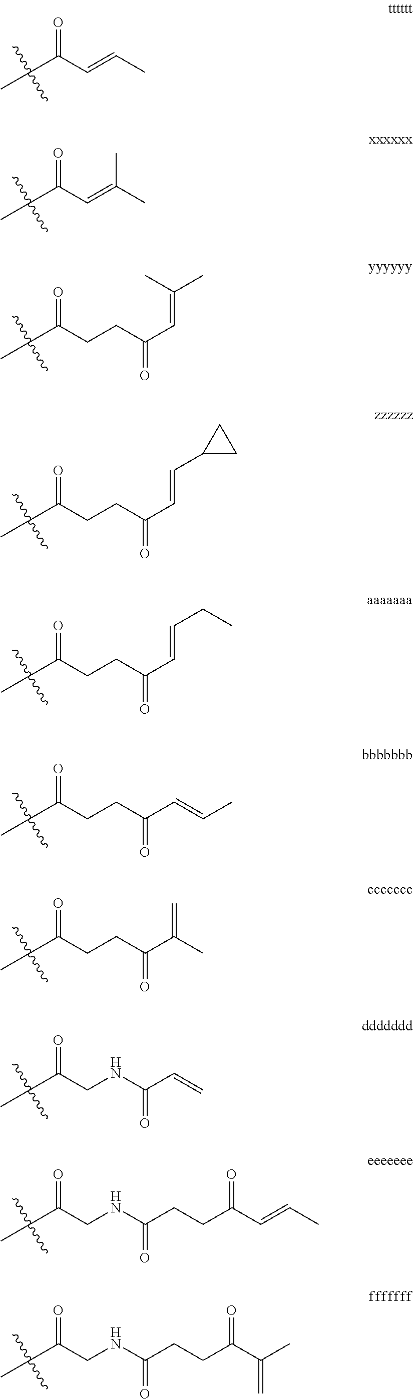

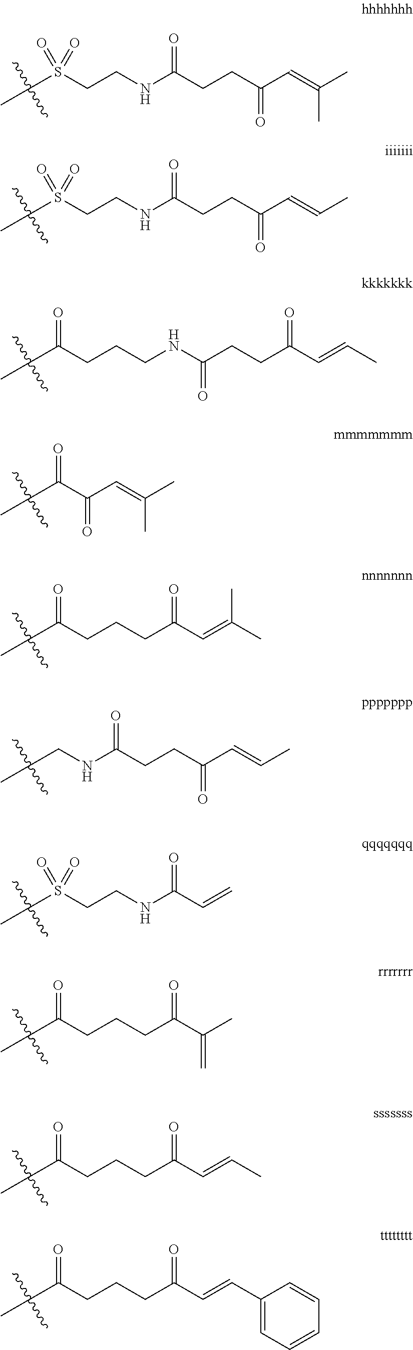

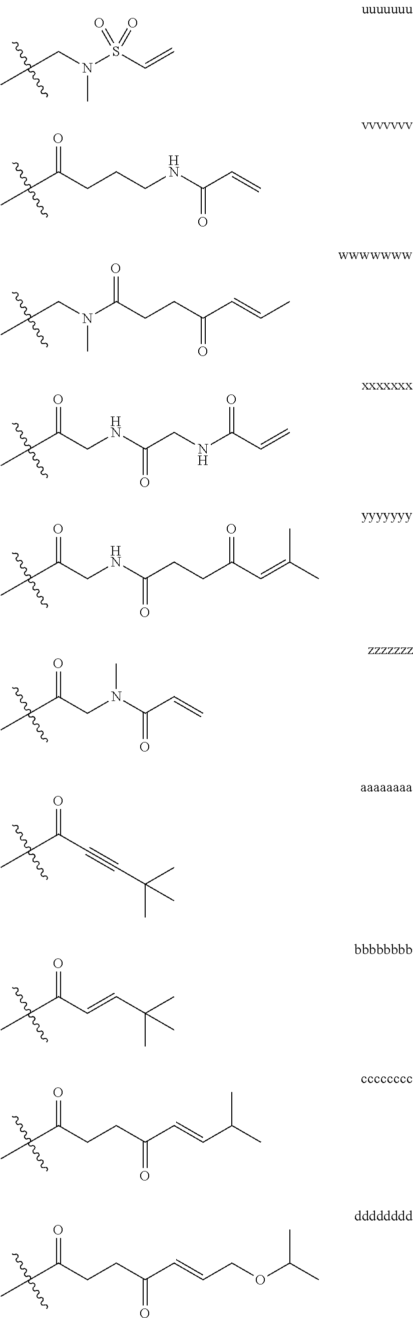

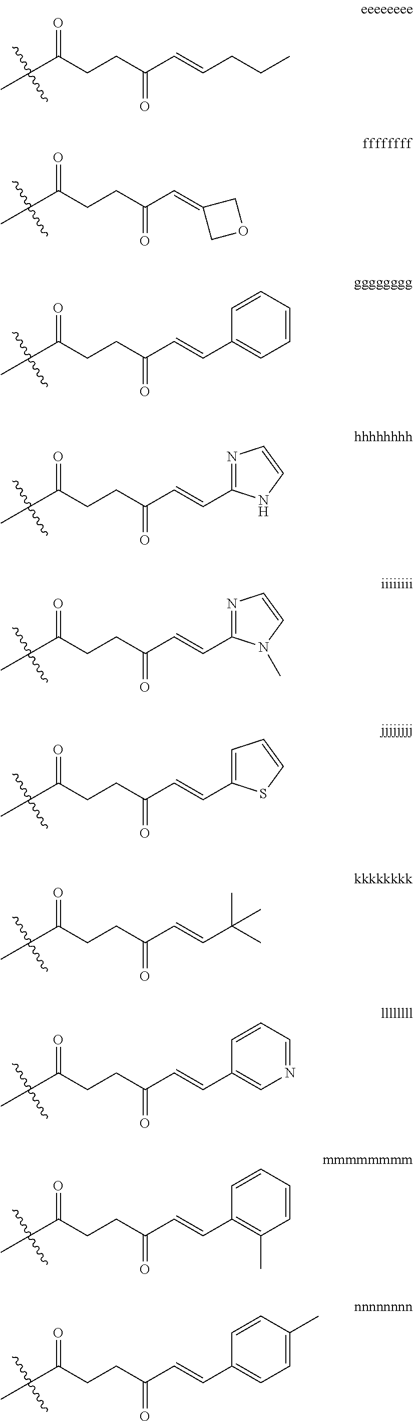

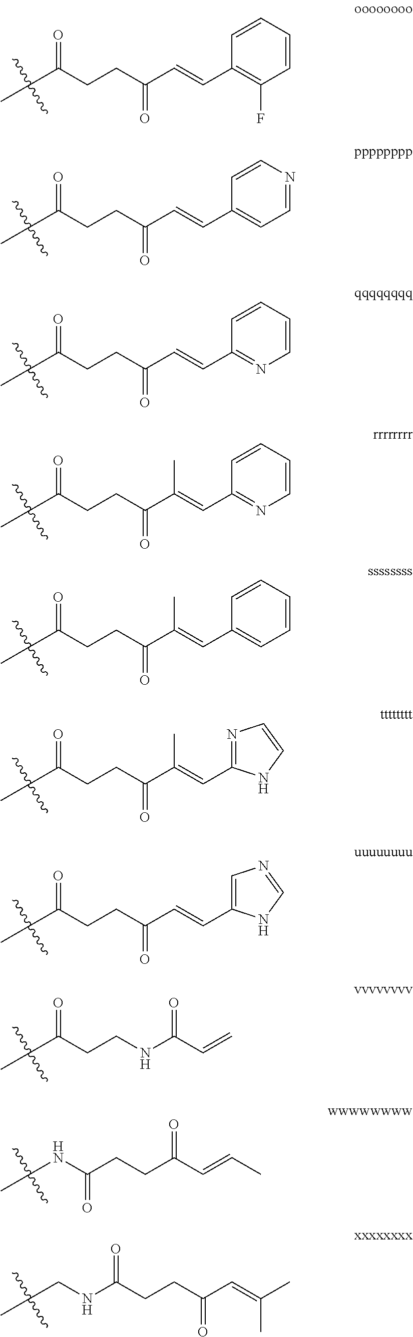

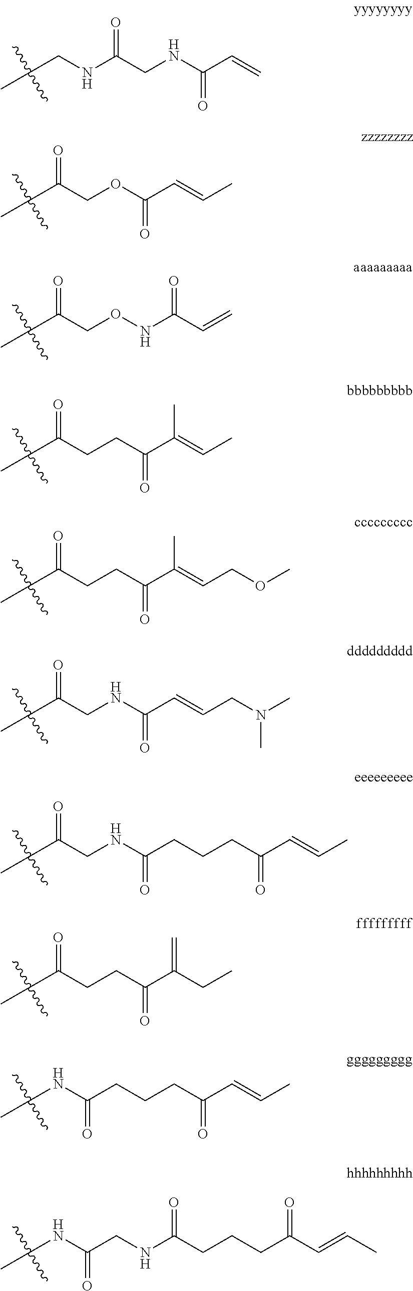

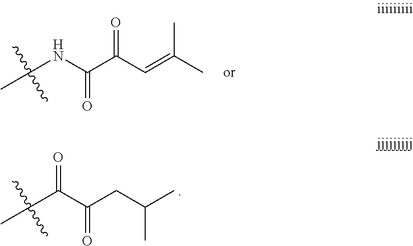

366. The compound according to claim 55, wherein R¹ is selected from:

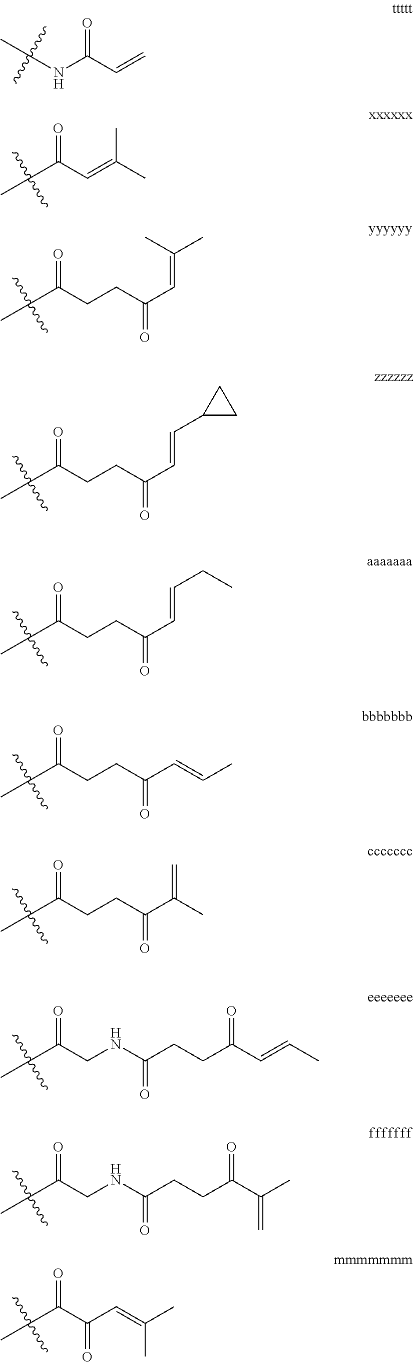

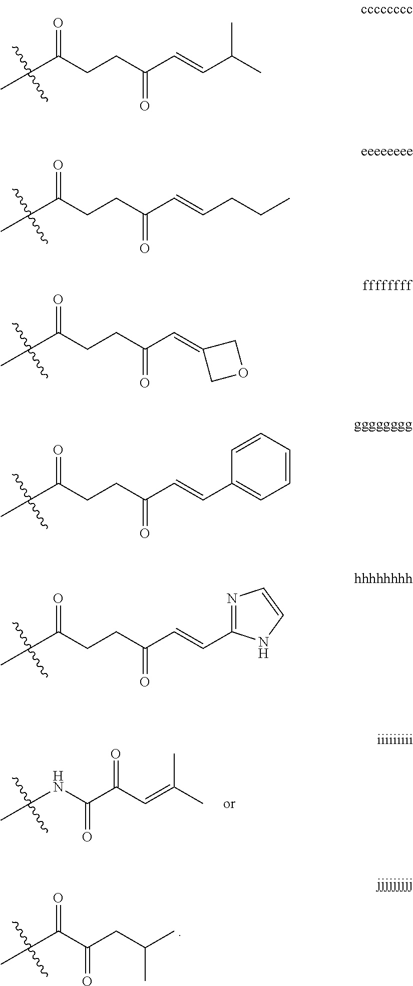

367. A composition comprising a compound according to claim 55, and a pharmaceutically acceptable adjuvant, carrier, or vehicle.

368. The composition according to claim 367, in combination with an additional therapeutic agent.

369. The composition according to claim 368, wherein the additional therapeutic agent is a chemotherapeutic agent.

370. A method for inhibiting one or more PI3 kinases, or a mutant thereof, activity in a biological sample comprising the step of contacting said biological sample with a compound according to claim 55.

371. A method for inhibiting one or more PI3 kinases, or a mutant thereof, activity in a patient comprising the step of administering to said patient a compound according to claim 55.

372. The method according to claim 371, wherein the one or more PI3 kinases, or a mutant thereof, activity is inhibited irreversibly.

373. The method according to claim 372, wherein the one or more PI3 kinases, or a mutant thereof, activity is inhibited irreversibly by covalently modifying Cys862 of PI3K-alpha, Cys2243 of MTOR, Cys838 of PI3K-alpha, Cys869 of PI3K-gamma, Cys815 of PI3K-delta, Cys841 of PI3K-beta, Class 1A, Cys1119 of PI3K-beta, Class 2, Cys3683 of DNA-PK, Cys2770 of ATM-Kinase, Cys2753 of ATM-Kinase, Cys1840 of PI4KA, Cys1844 of PI4KA, or Cys 1797 of PI4KA.

374. A method for treating a PI3Kα-mediated, a PI3Kγ-mediated, a PI3Kδ-mediated, a PI3Kβ-mediated, a PI3KC2β-mediated, an mTOR-mediated, a DNA-PK-mediated, an ATM-mediated and/or a PI4KIIIα-mediated disorder, disease, or condition in a patient in need thereof, comprising the step of administering to said patient a compound according to claim 55.

375-415. (canceled)


SEQUENCE LISTING (14 sequences)

SEQ ID NO: 1 - protein, 20 aa, Homo sapiens
QCKGGLKGALQFNSHTLHQW

SEQ ID NO: 2 - protein, 20 aa, Homo sapiens
PHCDTLHALIRDYREKKKIL

SEQ ID NO: 3 - protein, 7 aa, Homo sapiens
LPYGCLS

SEQ ID NO: 4 - protein, 7 aa, Homo sapiens
LPYGCIS

SEQ ID NO: 5 - protein, 7 aa, Homo sapiens
TPYGCLP

SEQ ID NO: 6 - protein, 7 aa, Homo sapiens
LPYGCLA

SEQ ID NO: 7 - protein, 7 aa, Homo sapiens
VIFRCFS

SEQ ID NO: 8 - protein, 20 aa, Homo sapiens
NKDSKPPGNLKECSPWMSDF

SEQ ID NO: 9 - protein, 20 aa, Homo sapiens
SQRSGVLEWCTGTVPIGEFL

SEQ ID NO: 10 - protein, 20 aa, Homo sapiens
RNTETRKRKLTICTYKVVPL

SEQ ID NO: 11 - protein, 20 aa, Homo sapiens
TAPGCGVIECIPDCTSRDQL

SEQ ID NO: 12 - protein, 20 aa, Homo sapiens
TAPGCGVIECIPDCTSRDQL

SEQ ID NO: 13 - protein, 20 aa, Homo sapiens
GQKISWQAAIFKVGDDCRQD

SEQ ID NO: 14 - protein, 11 aa, Homo sapiens
NSHTIMQIQCK